(19) United States
(12) Patent Application Publication
(10) Pub. No.: US 2009/0176742 A1
(43) Pub. Date: Jul. 9, 2009

(54) METHODS RELATING TO THE TREATMENT OF FIBROTIC DISORDERS

(75) Inventors: William R. HENDERSON, Seattle, WA (US); Michael KAHN, Altadena, CA (US)

Correspondence Address: NIXON PEABODY LLP - PATENT GROUP, 1100 CLINTON SQUARE, ROCHESTER, NY, 14604, US

(73) Assignees: UNIVERSITY OF WASHINGTON, Seattle, WA (US); INSTITUTE FOR CHEMICAL GENOMICS, Kirkland, WA (US)

(21) Appl. No.: 11/861,009

(22) Filed: Sep. 25, 2007

Related U.S. Application Data
(63) Continuation of application No. PCT/US2006/043626, filed on Nov. 8, 2006.
(60) Provisional application No. 60/734,476, filed on Nov. 8, 2005.

(51) Int. Cl. A61K 31/4985; A61K 31/675; A61K 31/538; A61K 31/53

(52) U.S. Cl. 514/81; 514/249; 514/230.5; 514/243

(57) ABSTRACT

The invention provides α-mimetic structure of formula (I), wherein A is —(C═O)—(CHR₃)— or —(C═O)—; B is —(NR₅)— or —(CHR₆)—; D is —(C═O)—(CHR₇)— or —(C═O)—; E is -(ZR₈)— or —(C═O)—, where Z is nitrogen or CH; —(XR₉)ₙ—, —(CHR₁₀)—(NR₆)—, —(C═O)—(XR₁₂)—, —(C═N—W—R₁)—, —(C═O)—, —(X—(C═O)—R₁₃)—, —(X—(C═O)—NR₁₃R₁₄)—, —(X—(SO₂)—R₁₃)—, or —(X—(C═O)—OR₁₃)—, where X is nitrogen or CH, and n=0 or 1; W is —Y(C═O)—, —(C═O)—(NH)—, —(SO₂)—, —(CHR₁₄)—, —(C═O)—(NR₁₅)—, substituted or unsubstituted oxadiazole, substituted or unsubstituted triazole, substituted or unsubstituted thiadiazole, substituted or unsubstituted 4,5-dihydrooxazole, substituted or unsubstituted 4,5-dihydrothiazole, substituted or unsubstituted 4,5-dihydroimidizole, or nothing, where Y is oxygen or sulfur; and R₁, R₂, R₃, R₄, R₅, R₆, R₇, R₈, R₉ R₁₀, R₁₁, R₁₂, R₁₃, R₁₄, and R₁₅ are the same or different and independently selected from an amino acid side chain moiety or derivative thereof, the remainder of the molecule, a linker and a solid support, and stereoisomers, salts, and prodrugs thereof, provided that where B is —(CHR₆) and W is —Y(C═O)—, —(C═O)—(NH)—, —(SO₂)—, —(CHR₁₄)—, or —(C═O)—(N₁₅)—, G cannot be —(CHR₉)—, —(NR₉)—, —(C═O)—(CHR₁₂)—, —(C═O)—(NR₁₂)—, or no atom at all. Additionally, the invention provides methods wherein α-mimetic compounds are used to treat a disease or pathological condition.

### CROSS REFERENCE TO RELATED APPLICATION(S)

The present application is a continuation of PCT International Patent Application No. PCT/US2006/043626, filed Nov. 8, 2006, which claims priority from U.S. Provisional Application No. 60/734,476, filed Nov. 8, 2005, both of which are hereby incorporated by reference in their entirety.

The subject matter of this application was made with support from the United States Government under The National Institutes of Health, Grant No. R01 HL073722. The U.S. Government may have certain rights.

### TECHNICAL FIELD

The present invention relates generally to α-helix mimetic structures and to a chemical library relating thereto. The invention also relates to applications in the treatment of fibrotic diseases and pharmaceutical compositions comprising them.

### BACKGROUND OF THE INVENTION

Fibrosis can occur in the lung, liver, kidney, eye, heart, and other major organs of the body. Fibrosis can be due to toxic or infectious injury, such as cigarette smoke to the lungs or viral hepatitis infection of the liver. The cause of some fibrotic diseases is unknown, which is the case with idiopathic pulmonary fibrosis.

Idiopathic pulmonary fibrosis (IPF) is a chronic and insidious inflammatory disease of the lung that kills most of its victims within five years after diagnosis. IPF afflicts 83,000 Americans and more than 31,000 new cases develop each year. It is believed that death due to IPF is greatly underreported and the considerable morbidity of IPF is not recognized. IPF represents just one of the many fibrotic diseases that occurs as a result of chronic inflammation.

It is estimated by the United States government that 45% of all deaths in the U.S. can be attributed to fibrotic disorders. However, no drugs have been approved for the treatment of any fibrotic disease in the United States. Research and development is desperately needed to provide treatments to those afflicted with fibroproliferative diseases. The present invention fulfills these needs, and provides further related advantages by providing conformationally constrained compounds which mimic the secondary structure of α-helix regions of biologically active peptides and proteins.

### SUMMARY OF THE INVENTION

In brief, the present invention is directed to treatment of fibrotic disease using conformationally constrained compounds, which mimic the secondary structure of α-helix regions of biologically active peptides and proteins. This invention also discloses libraries containing such compounds, as well as the synthesis and screening thereof.

Provided is a compound having the following general formula (I):

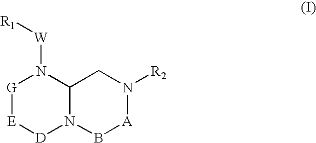

wherein A is —(C═O)—CHR₃—, or —(C═O), B is N—R₅— or —CHR₆—, D is —(C═O)—(CHR₇)— or —(C═O)—, E is -(ZR₈)— or (C═O), G is —(XR₉)ₙ—, —(CHR₁₀)—(NR₆)—, —(C═O—(XR₁₂)—, —(C═N—W—R₁)—, —(C═O)—, X—(C═O)—R₁₃, X—(C═O)—NR₁₃R₁₄, X—(SO₂)—R₁₃, or X—(C═O)—OR₁₃, W is —Y(C═O)—, —(C═O)NH—, —(SO₂)—, —CHR₁₄, (C═O)—(NR₁₅)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, or nothing, Y is oxygen or sulfur, X and Z is independently nitrogen or CH, n=0 or 1; and R₁, R₂, R₃, R₄, R₅, R₆, R₇, R₈, R₉ R₁₀, R₁₁, R₁₂, R₁₃, R₁₄, and R₁₅ are the same or different and independently selected from an amino acid side chain moiety or derivative thereof, the remainder of the molecule, a linker and a solid support, and stereoisomers, salts, and prodrugs thereof, provided that where B is CH and W is —Y(C═O)—, —(C═O)NH—, —(SO₂), —CHR₁₄, or (C═O)—(NR₁₅)—, G cannot be CHR₉, NR₉, (C═O)—CHR₁₂, (C═O)—NR₁₂, or no atom at all.

Also provided is a compound, salts, and prodrugs thereof of formula (I), wherein R₁, R₂, R₃, R₄, R₅, R₆, R₇, R₈, R₉, R₁₀, R₁₁, R₁₂, R₁₃, R₁₄, are R₁₅ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl.

Further provided is the compound, salts, and prodrugs thereof of compound (I) wherein A is —(CHR₃)—(C═O)—, B is —(NR₄)—, D is (C═O)—, E is -(ZR₆)—, G is —(C═O)—(XR₉)—, and the compound has the following general formula (III):

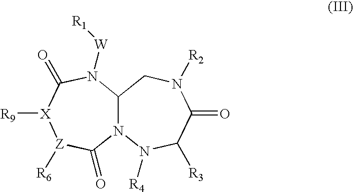

wherein R₁, R₂, R₄, R₆, R₉, W and X are as defined in claim 1, Z is nitrogen or CH (when Z is CH, the X is nitrogen).

Also provided is a compound, salts, and prodrugs thereof of formula (I) wherein A is —O—CHR₃—, B is —NR₄—, D is —(C═O)—, E is -(ZR₆)—, Gi is (XR₇)ₙ—, the α-helix mimetic compounds of this invention have the following formula (IV):

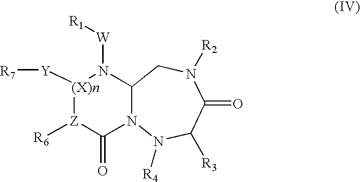

wherein R₁, R₂, R₄, R₆, R₇, R₈, W, X and n are as defined above, Y is —C═O, —(C═O)—O—, —(C═O)—NR₈, —SO₂—, or nothing, and Z is nitrogen or CH (when Z is nitrogen, then n is zero, and when Z is CH, then X is nitrogen and n is not zero). In a preferred embodiment, R₁, R₂, R₆, R₇, and R₈ represent the remainder of the compound, and R₄ is selected from an amino acid side chain moiety. In this case, R₆ or R₇ may be selected from an amino acid side chain moiety when Z and X are CH, respectively.

Further provided is a compound, salts, and prodrugs thereof of formula (I) wherein A is —(C═O), B is —(CHR₆)—, D is —(C═O)—, E is -(ZR₈)—, and G is —(NH)— or —(CH₂)—, and W is a substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, the α-helix mimetic compounds of this invention have the following formula (V):

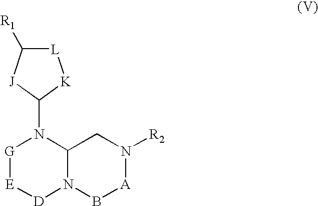

wherein K is nitrogen, oxygen, or sulfur, L is nitrogen, oxygen, —(CH)—, or —(CH₂)—, J is nitrogen, oxygen, or sulfur, Z is nitrogen or CH, and R₁, R₂, R₆, R₈, and R₁₃ are selected from an amino acid side chain moiety.

Also provided is a compound having the general formula (VI):

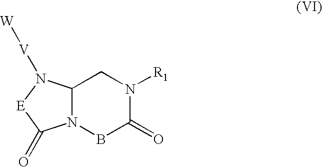

wherein B is —(CHR₂)—, —(NR₂)—, E is —(CHR₃)—, V is —(XR₄)— or nothing, W is —(C═O)—(XR₅R₆), —(SO₂)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, X is independently nitrogen, oxygen, or CH, and R₁, R₂, R₃, R₄, R₅ and R₆ are selected from an amino acid side chain moiety or derivative thereof, the remainder of the molecule, a linker and solid support, and stereoisomers, salts, and prodrugs thereof.

Further provided is a compound, salts, and prodrugs thereof of formula (I), wherein R₁, R₂, R₃, R₄, R₅, R₆, R₇, R₈, R₉, R₁₀, R₁₁, R₁₂, R₁₃, R₁₄, are R₁₅ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl. Further provided is a compound, salts, and prodrugs thereof of wherein B is —(CH)—(CH₃), E is —(CH)—(CH₃), V is —(XR₄)— or nothing, and W is substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, and X is independently introgen or CH, the compounds have the following general formula (VII):

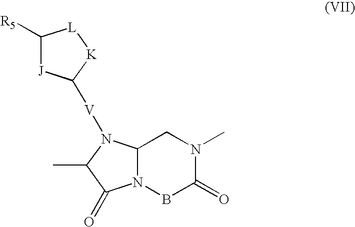

Wherein K is nitrogen, oxygen, or sulfur, L is nitrogen, oxygen, —(CH)—, or —(CH₂)—, J is nitrogen, oxygen, or sulfur, and R₅ is independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₅alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄allyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl.

Provided is a pharmaceutical composition comprising a compound of the following general formula (I)

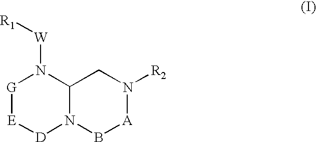

wherein A is —(C═O)—CHR₃—, or —(C═O), B is N—R₅— or —CHR₆—, D is —(C═O)—(CHR₇)— or —(C═O)—, E is -(ZR₈)— or (C═O), G is —(XR₉)ₙ—, —(CHR₁₀)—(NR₆)—, —(C═O)—(XR₁₂)—, -(or nothing)-, —(C═O)—, X—(C═O)—R₁₃, X—(C═O)—NR₁₃R₁₄, X—(SO₂)—R₁₃, or X—(C═O)—OR₁₃, W is —Y(C—O)—, —(C═O)NH—, —(SO₂)—, —CHR₁₄, (C═O)—(NR₁₅)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, or nothing, Y is oxygen or sulfur, X and Z is independently nitrogen or CH, n=0 or 1; and R₁, R₂, R₃, R₄, R₅, R₆, R₇, R₈, R₉, R₁₀, R₁₁, R₁₂, R₁₃, R₁₄, and R₁₅ are the same or different and independently selected from an amino acid side chain moiety or derivative thereof, the remainder of the molecule, a linker and a solid support, and stereoisomers salts, and prodrugs thereof, and a pharmaceutically acceptable carrier.

Also provided is a pharmaceutical composition comprising the compound of formula (I), wherein R₁, R₂, R₃, R₄, R₅, R₆, R₇, R₈, R₉, R₁₀, R₁₁, R₁₂, R₁₃, R₁₄, are R₁₅ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄akylamino, C₁₋₄dialkylamino, halogen, perfluoro C₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl. Further provided is a pharmaceutical composition of formula (I) wherein A is —(CHR₃)—(C═O)—, B is —(NR₄)—, D is (C═O)—, E is -(ZR₆)—, G is —(C═O)—(XR₉)—, and the compound has the following general formula (III):

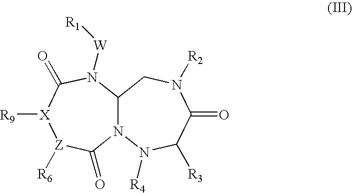

wherein Z is nitrogen or CH (when Z is CH, the X is nitrogen).

Also provided is a pharmaceutical composition of formula (I) wherein A is —O—CHR₃—, B is —NR₄—, D is —(C—O)—, E is -(ZR₆)—, Gi is (XR₇)ₙ—, the α-helix mimetic compounds have the following formula (IV):

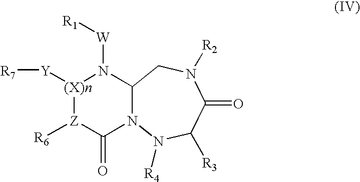

wherein R₁, R₂, R₄, R₆, R₇, R₈ W, X and n are as defined above, Y is —C═O, —(C—O)—O—, —(C═O)—NR₈, —SO₂—, or nothing, and Z is nitrogen or CH (when Z is nitrogen, then n is zero, and when Z is CH, then X is nitrogen and n is not zero). In a preferred embodiment, R₁, R₂, R₆, R₇, and R₈ represent the remainder of the compound, and R₄ is selected from an amino acid side chain moiety. In this case, R₆ or R₇ may be selected from an amino acid side chain moiety when Z and X are CH respectively. Also provided is a pharmaceutical composition wherein A is —(C═O), B is —(CHR₆)—, D is —(C═O)—, E is -(ZR₈)—, and G is —(NH)— or —(CH₂)—, and W is a substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, the α-helix mimetic compounds of this invention have the following formula (V):

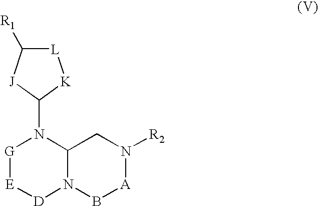

wherein K is nitrogen, oxygen, or sulfur, L is nitrogen, oxygen, —(CH)—, or —(CH₂)—, J is nitrogen, oxygen, or sulfur, Z is nitrogen or CH, and R₁, R₂, R₆, R₈, and R₁₃ are selected from an amino acid side chain moiety.

Further provided is a pharmaceutical composition comprising a compound having the general formula (VI):

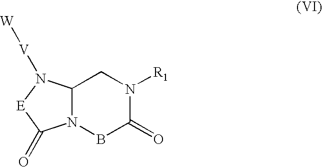

wherein B is —(CHR₂)—, —(NR₂)—, E is —(CHR₃)—, V is —(XR₄)— or nothing, W is —(C═O)—(XR₅R₆), —(SO₂)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, X is independently nitrogen, oxygen, or CH, and R₁, R₂, R₃, R₄, R₅ and R₆ are selected from an amino acid side chain moiety or derivative thereof, the remainder of the molecule, a linker and solid support, and stereoisomers, salts and prodrugs thereof. In this pharmaceutical composition, wherein R₁, R₂, R₃, R₄, R₅, R₆, R₇, R₈, R₉, R₁₀, R₁₁, R₁₂, R₁₃, R₁₄, are R₁₅ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl substituted naphthyl (where the substituents are independently selected from one or more of amino, amidino, guanidino, hydrazino, amidrazonyl, C₁₋₄alkylamine, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₅alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alyklaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl. In certain embodiments, wherein B is —(CH)—(CH₃), E is —(CH)—(CH₃), V is —(XR₄)— or nothing, and W is substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, and X is independently introgen or CH, the compounds have the following general formula (VII):

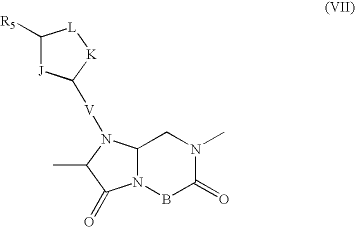

wherein K is nitrogen, oxygen, or sulfur, L is nitrogen, oxygen, —(CH)—, or —(CH₂)—, J is nitrogen, oxygen, or sulfur, and R₅ is independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy or nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, or methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl.

Provided is a compound selected from the group consisting of Compounds 1-2217, and pharmaceutical composition a comprising at least one compound of Compounds 1-2217. The pharmaceutical composition may comprise an effective amount of the compound and a pharmaceutically acceptable carrier. Also provided are diasteric and enantiomeric stereo isomers of Compounds 2203-2217.

The present invention is also directed to libraries containing compounds of formula (I) above, as well as methods for synthesizing such libraries and methods for screening the same to identify biologically active compounds. Compositions containing a compound of this invention in combination with a pharmaceutically acceptable carrier or diluent are also disclosed.

Especially, the present invention relates pharmaceutical compositions containing compounds disclosed herein for treating disorders including fibrosis which are associated with TGF-β signaling pathway. It further relates to methods for treating disorders including fibrosis which are associated with TGF-β signaling pathway.

These and other aspects of this invention will be apparent upon reference to the attached figures and following detailed description. To this end, various references are set forth herein, which describe in more detail certain procedures, compounds and/or compositions, and are incorporated by reference in their entirety.

### BRIEF DESCRIPTION OF THE DRAWINGS

FIG. 1A-Z shows the chemical structures of compounds 1-200.

FIG. 2A-2AD shows the chemical structures of compounds 201-400.

FIG. 3A-3AC shows the chemical structures of compounds 401-600.

FIG. 4A-4Y shows the chemical structures of compounds 601-800.

FIG. 5A-5Y shows the chemical structures of compounds 801-1000.

FIG. 6A-6Y shows the chemical structures of compounds 1001-1200.

FIG. 7A-7Z shows the chemical structures of compounds 1201-1400.

FIG. 8A-8AC shows the chemical structures of compounds 1401-1600.

FIG. 9A-9AE shows the chemical structures of compounds 1601-1800.

FIG. 10A-10AA shows the chemical structures of compounds 1801-2000.

FIG. 11A-11AA shows the chemical structures of compounds 2001-2200.

FIG. 12A-12C shows the chemical structures of diasteric and enantiomeric stereo isomers of Compounds 2203-2217.

FIG. 13. FIG. 13A shows the structure of the compound ASN 06387747. FIG. 13B shows the structure of the compound ICG001. FIG. 13C shows the structures of ASN 06387747 (green) and ICG001 (red) superimposed. In accordance with an certain embodiments of the present invention, each compound has three pharmacophore rings. Distances measured from the center of each pharmacophore ring may be based on a conformation generated by flexible alignment calculations. As shown in this figure, the distance between F1 and F4 is approximately 9.6 Å, the distance between F1 and F6 is approximately 9.2 Å, and the distance between F4 and F6 is approximately 10.3 Å.

FIG. 14. FIG. 14A-C depicts lung sections taken from Bat-Gal transgenic mice given intratracheal saline or bleomycin and either treated with ICG-001 (5 mgs/Kg/day subcutaneously) or saline as vehicle control. The lungs were sectioned and stained with X-Gal (blue color.) FIG. 14A, intratracheal bleo+saline; FIG. 14B, intracheal bleo+ICG-001; FIG. 14C, saline+saline.

FIG. 15. FIG. 15 depicts lung sections taken from C57/B16 mice treated with intratracheal bleomycin (lower left) or saline (upper left) for 5 days and stained with trichrome (red color) to stain collagen.

FIG. 16. FIG. 16 shows RT-PCR data for S100A4, which was increased in the bleomycin treated mice.

FIG. 17. FIG. 17 shows RT-PCR data for collagen1A2, which was increased in the bleomycin treated mice.

FIG. 18. FIG. 18 is a graph showing that over the 25 days of treatment, ICG-001 reversed fibrosis.

FIG. 19. FIG. 19 is a photograph showing that over the 25 days of treatment, ICG-001 reversed fibrosis.

FIG. 20. FIG. 20 depicts a Western blots for S100A4 (also know as FSP-1 or fibroblast specific protein-1) and E-Cadherin performed on whole cell lysates of IPF patient fibroblasts cultured in RPMI1640+10% FBS for 2 days and treated with ICG-001.

FIG. 21. FIG. 21 is a bar graph depicting decreased S100A4 expression in whole cell lysates of ATCC IPF cells and IPF patient cells cultured in RPMI1640+10% FBS for 2 days and treated with ICG-001.

FIG. 22. FIG. 22A-C shows that aquaporin 5 expression was greatly increased by ICG-001 expression. Bleomycin alone, FIG. 22A; bleomycin and ICG-001, FIG. 22B; saline FIG. 22C.

FIG. 23. FIG. 23A-C shows that ICG-001 prevented interstitial fibrosis. FIG. 23A, saline treatment; FIG. 23B, bleomycin treatment; and FIG. 23C, bleomycin and ICG-001 treatment.

FIG. 24. FIG. 24A-C shows that ICG-001 prevented alveolar fibrosis. FIG. 24A, saline treatment; FIG. 24B, bleomycin treatment; and FIG. 24C, bleomycin and ICG-001 treatment.

FIG. 25. FIG. 25 is a diagram of autocrine and paracrine Wnt signaling in the lung. Several Wnt ligands are expressed in either the epithelium or mesenchyme during development and in the adult. β-catenin is expressed in both alveolar epithelium as well as the adjacent mesenchyme.

FIG. 26A-26E shows the chemical structures of compounds 2203-2217.

### DETAILED DESCRIPTION OF THE INVENTION

Transforming growth factor β (TGF-β), a key mediator in the development of fibrosis, is important in cell proliferation and differentiation, apoptosis, and deposition of extracellular matrix (ECM). TGF-β signaling activates both the Smad and AP-1 transcription pathways. TGF-β in the airways of patients with pulmonary fibrosis (PF) may function initially as a “healing molecule” involved in the diminution of initial airway inflammation and in tissue repair. However, with continued inflammatory response such as may occur in PF, the balance may be shifted, to excessive ECM deposition and development of airway fibrosis.

Fibroproliferative diseases are generally caused by the activation of resident stellate cells which are found in most organs. This activation of stellate cells leads to their conversion to myofibroblasts which display characteristics of muscle and non-muscle cells. Activated stellate cells initiate inflammatory signals, principally mediated through TGF-β. Inflammatory cytokines and mediators in addition to TGF-β, lead to proliferation of myofibroblasts. Stellate-derived myofibroblasts proliferate and replace healthy, functional organ cells with extra-cellular matrix that exhibit muscle and connective tissue traits. Ultimately, organ failure results when the nonfunctional fibrotic honeycomb matrix replaces a critical number of healthy cells.

The initial cause of fibrosis is believed to be the result of injury or insult to organ tissues. This cellular injury to organ tissues can often be traced to toxic or infectious agents. Pulmonary fibrosis, or interstitial lung disease, is often the result of smoking, chronic asthma, chronic obstructive pulmonary disease (COPD) or pneumonia. Fibrosis affects nearly all tissues and organ systems. Non-limiting examples of disorders in which fibrosis is a major cause of morbidity and mortality are listed below.

### Major-Organ Fibrosis

Interstitial lung disease (ILD) includes a wide range of distinct disorders in which pulmonary inflammation and fibrosis are the final common pathway of pathology. There are more than 150 causes of ILD, including sarcoidosis, silicosis, adverse drug reactions, infections and collagen vascular diseases, i.e. rheumatoid arthritis and systemic sclerosis (scleroderma). Idiopathic pulmonary fibrosis (IPF) is the most common type of ILD. Liver cirrhosis has similar causes to ILD, with viral hepatitis, schistosomiasis and chronic alcoholism being the major causes worldwide.

Kidney disease including diabetes can damage and scar the kidneys, which leads to progressive loss of function. Untreated hypertension can also contribute to the fibroproliferation of the kidneys.

Heart disease associated with scar tissue can impair the heart's pumping ability. Eye Disease including macular degeneration and retinal and vitreal retinopathy can impair vision. Chronic pancreatitis is an irreversible disease of the pancreas characterized by chronic inflammation and fibrosis which leads to the loss of endocrine and exocrine function. Fibroproliferative disorders include systemic and local seleroderrna. Scleroderma is a chronic connective tissue disease that may be localized or systemic, and may have an affect in many organs and tissues of the body.

Keloids and hypertrophic scars, which can occur after surgery, traumatic wounds, burns, or even scratches. They manifest as an overgrowth of scar tissue at the site of injury. Atherosclerosis and restenosis. Restenosis refers to the re-narrowing of a coronary artery after angioplasty to treat atherosclerosis. Scarring associated with trauma can be associated with overgrowth of scar tissue at the site of the trauma-related injury. Surgical complications can lead to fibrosis in any organ in which scar tissue and fibroproliferation result from the surgical procedures.

Chemotherapy induced fibrosis can occur in, for example, the lungs following chemotherapy, manifests as pulmonary fibrosis, and can be severe enough to require lung transplant, even in cases where the underlying malignancy did not affect the lungs.

Radiation-induced fibrosis (RIF) is a serious and common complication of radiation therapy that may cause chronic pain, neuropathy, limited movement of joints, and swelling of the lymph nodes. It occurs most often in breast, head, neck, and connective tissues. RIF may develop from 4-6 months to 1-2 years following exposure to radiation therapy, and it becomes more severe over time. Risk factors for developing RIF include high radiation dose, large volumes of tissue exposed to radiation, and radiation combined with surgery, chemotherapy, or both.

Burns can lead to fibrosis when there is an overproduction of ECM proteins. Excessive ECM deposition causes the tissue to become fibrotic.

### Pulmonary Fibrosis

Pulmonary fibrosis destroys the lung's ability to transport oxygen and other gases into or out of the blood. This disease modifies the delicate and elastic tissues of the lung, changing these tissues into thicker, stiff fibrous tissue. This change or replacement of the original tissue is similar to the permanent scarring that can occur to other damaged tissues. Scarring of the lung reduces the lung's ability to allow gases to pass into or out of the blood (i.e. oxygen, carbon dioxide). Gradually, the air sacs of the lungs become replaced by fibrotic tissue. When the scar forms, the tissue becomes thicker causing an irreversible loss of the tissue's ability to transfer oxygen into the bloodstream. Symptoms include shortness of breath, particularly with exertion; chronic dry, hacking cough; fatigue and weakness; discomfort in the chest, loss of appetite; and rapid weight loss.

Several causes of pulmonary fibrosis are known and they include occupational and environmental exposures. Many jobs, particularly those that involve mining or that expose workers to asbestos or metal dusts, can cause pulmonary fibrosis. Workers doing these kinds of jobs may inhale small particles (like silica dusts or asbestos fibers) that can damage the lungs, especially the small airways and air sacs, and cause the scarring associated with fibrosis. Agricultural workers also can be affected. Some organic substances, such as moldy hay, cause an allergic reaction in the lung. This reaction is called Farmer's Lung and can cause pulmonary fibrosis. Other fumes found on farms are directly toxic to the lungs.

Another cause is Sarcoidosis, a disease characterized by the formation of granulomas (areas of inflammatory cells), which can attack any area of the body but most frequently affects the lungs.

Certain medicines may have the undesirable side effect of causing pulmonary fibrosis, as can radiation, such as treatment for breast cancer. Connective tissue or collagen diseases such as rheumatoid arthritis and systemic sclerosis are also associated with pulmonary fibrosis.

Although genetic and familial factors may be involved, this cause is not as common as the other causes listed above.

In Chronic Obstructive Pulmonary Disease (COPD), connective tissue proliferation and fibrosis can characterize severe COPD. COPD can develop as a result of smoking or chronic asthma.

### Idiopathic Pulmonary Fibrosis (IPF)

When all known causes of interstitial lung disease have been ruled out, the condition is called “idiopathic” (of unknown origin) pulmonary fibrosis (IPF). Over 83,000 Americans are living with IPF, and more than 31,000 new cases develop each year. This debilitating condition involves scarring of the lungs. The lungs' air sacs develop scar, or fibrotic tissue, which gradually interferes with the body's ability to transfer the oxygen into the bloodstream, preventing vital organs and tissue from obtaining enough oxygen to function normally.

There are several theories as to what may cause IPF, including viral illness and allergic or environmental exposure (including tobacco smoke). These theories are still being researched. Bacteria and other microorganisms are not thought to be the cause of IPF. There is also a familial form of the disease, known as familial idiopathic pulmonary fibrosis. Additional research is being done to determine whether there is a genetic tendency to develop the disease, as well as to determine other causes of IPF.

Patients with IPF suffer similar symptoms to those with pulmonary fibrosis when their lungs lose the ability to transfer oxygen into the bloodstream. The symptoms include shortness of breath, particularly during or after physical activity; spasmodic, dry cough; gradual, unintended weight loss; fatigue and weakness; chest discomfort; clubbing, or enlargement of the ends of the fingers (or sometimes the toes) due to a buildup of tissue. These symptoms can greatly reduce IPF patients' quality of life. Pulmonary rehabilitation, and oxygen therapy can reduce the lifestyle-altering effects of IPF, but do not provide a cure.

In order to develop a treatment for fibrotic disease, it is important to focus on the common pathway to the ultimate pathology that is shared by the disease states, regardless of cause or of tissue in which it is manifested. Several components of the causative pathway are discussed below, particularly in relation to the role of β-catenin.

### Other Pathological Conditions

Survivin, an inhibitor of apoptosis, is implicated in pulmonary hypertension. CK2 kinase activity has been shown to promote cell survival by increasing survivin expression via β-catenin-Tcf/Lef-mediated transcription. Tapia, J. C. et al., Proc. Nat. Acad. Sci. U.S.A. 103:15079-84 (2006). This pathway therefore provides another opportunity to utilize the present compounds to alter the β-catenin-mediated gene transcription processes.

McMurtry, M. S. et al., J. Clin. Invest. 115:1461-1463 (2005) reported that survivin was expressed in the pulmonary arteries of patients with pulmonary arterial hypertension, but not in the pulmonary arteries of patients without pulmonary arterial hypertension. Comparable results were found in rats treated with monocrotaline to induce pulmonary arterial hypertension. In the rats, survival was prolonged and the pulmonary arterial hypertension was reversed by gene therapy with inhalation of an adenovirus carrying a survivin mutant with dominant-negative properties.

Survivin expression is upregulated in hyperproliferative neovasculature (Simosa, H. F. et al., J. Vasc. Curg. 41:682-690, 2005). Survivin was specifically expressed in human atherosclerotic plaque and stenotic vein grafts. In a rabbit model of hyperplasia after balloon injury of iliofemoral arteries, treatment with a phosphorylation-defective survivin mutant vector reduced the neointimal area. The correlation between survinin expression and regulation of a smooth muscle cell phenotype after vascular injury points to survivin as a target for therapy in treating vascular disease.

Survivin is amenable to targeting by administration of a compound disclosed herein via one or more of the routes as described herein. Without being bound by a particular mode of action, the compounds disclosed herein can be administered in the form of coated stents, for example in connection with angioplasty. The methods for preparing coated stents are described in the art and would be modified as needed for use with the compounds of the invention. For example, U.S. Pat. No. 7,097,850 discloses and teaches methods of coating a stent with a variety of bioactive compounds. U.S. Pat. No. 7,087,078 discloses methods of preparing a stent with at least one active ingredient. Both coronary and peripheral stents are amenable to incorporating one or more compounds disclosed herein. Further teachings regarding drug-coated stents is available in Grube, E. et al., Herz 29:162-6 (2004) and W. L. Hunter, Adv Drug Deliv Rev. 58:347-9 (2006).

Bone marrow cells contribute to transplant-associated atherosclerosis (Sata, M., Trends Cardiovasc. Med. 13:249-253, 2003). Bone marrow cells also contribute to the pathogenesis of lesion formation after mechanical vascular injury (Sata, M. et al., Nat. Med. 8:403-409, 2002). Thus, by treating atherosclerosis and vascular damage with one of more compounds of the invention, reduction in vascular lesion formation can be accomplished.

Survivin also plays a role in vein graft hyperplasia (Wang, G. J. et al., Arterioscler. Thromb. Vasc. Biol. 25:2091-2087, 2005). Bypass grafts often develop intimal hyperplasia, a fibroproliferative lesion characterized by intimal thickening. Rabbit vein grafts were treated with adenoviral survivin constructs. Transgene expression was demonstrated in all the adenovirus-treated grafts. Treatment with a dominant negative mutant adenovirus decreased cellular proliferation in the early phase of graft remodeling. The data provide evidence for an important role of survivin in the regulation of vein graft remodeling in this system as well, and further support a role for the compounds of the invention in conjunction with bypass grafts.

Lymphangioleiomyomatosis (LAM) is a disease that occurs in some patients with tuberous sclerosis complex (Moss, J. et al., Am. J. Respir. Crit. Care Med. 163:669-671, 2001). Cystic lung disease in LAM is characterized by abnormal smooth muscle cell proliferation. Compounds disclosed herein are expected to find use in regulating and alleviating the cell proliferation, thus moderating the clinical symptoms.

### The Role of TGF-β

In pulmonary fibrosis, the normally thin lung tissue is replaced with thick, coarse scar tissue that impairs the flow of oxygen into the blood and leads to a loss of lung function. A growing body of research suggests that excess TGF-β is the immediate cause of the fibrosis. This over-expression of TGF-β has been shown to cause pulmonary fibrosis in mice. An abnormally high TGF-β signal causes healthy epithelial cells in the lung to die via apoptosis. Cell death leads to the replacement of healthy lung tissue by thick, poor functioning scar tissue. Apoptosis of healthy epithelial cells is required prior to the development of pulmonary fibrosis (Elias et al). One form of treatment of fibrotic lung disorders involves administering drugs that specifically inhibit TGF-β, which in turn blocks apoptosis, preventing the formation of fibrotic tissue in the lung. However, for reasons discussed below, TGF-β itself may not be an ideal therapeutic target.

TGF-β is a member of the transforming growth factor β superfamily which consists of secreted polypeptide signaling molecules involved in cell proliferation and differentiation, apoptosis, deposition of extracellular matrix (ECM) and cell adhesion. TGF-β is a potent inhibitor of cell growth, and has immunosuppressive properties. However, TGF-β also causes the deposition of ECM components leading to fibrosis. A role for TGF-β as a key mediator in the development of fibrosis relates to its ability to act as a chemoattractant for fibroblasts, stimulate fibroblast procollagen gene expression/collagen protein synthesis, and inhibit collagen breakdown. TGF-β further stabilizes the ECM by inhibiting the expression of ECM proteases and stimulating the expression of ECM protease inhibitors. The fibrinolysis system is essential in ECM accumulation and fibrosis. Inhibition of fibrinolysis results in the accumulation of fibrin and ECM. Plasminogen activator inhibitor-1 (PAI-1) is the key inhibitor of fibrinolysis. The PAI-promoter contains several transcription factor binding sites including an AP-1 and Smad binding elements that promote PAI-1 induction by TGF-β. PAI-1 is the primary inhibitor of both tissue-type (TPA) and urokinase-type plasminogen (uPA) activator. Thus, TGF-β and PAI-1 work in tandem to produce the characteristic tissue of fibrosis.

In the bleomycin-induced model of pulmonary fibrosis (PF), mice in which the PAI-1 gene is deleted are protected from developing PF. Additionally, adenovirus-mediated transfer of the uPA gene to the lung significantly reduces the production of lung hydroxyproline and attenuated the bleomycin-induced increase in lung collagen, both hallmarks of fibrosis. The TGF-β signaling pathway is complex. TGF-β family members bind to specific pairs of receptor serine/threonine kinases. Upon binding, the ligand acts to assemble two type I and two type II receptors into a complex. The type II receptor phosphorylates the type I receptor that subsequently phosphorylates the intracellular substrates Smad 2 and Smad3. This complex then binds Smad 4 and translocates to the nucleus for signal propagation. TGF-β can also activate AP-1 transcription via the MAPK pathway. TGF-β may originally act as a “healing molecule” in the lung or liver after initial inflammation and injury to the tissue. However, with continued inflammation/injury the balance may be shifted to excessive fibroproliferation and ECM deposition, leading to an “endless healing” process and development of fibrosis. Thus, complete inhibition of TGF-β could initially undermine the healing process.

TGF-β is highly expressed in airway epithelium and macrophages of small airways in patients with COPD. Using anti-inflammatory therapies, such as corticosteroids and interferon-γ, to treat PF has been disappointing due to variable efficacy and significant adverse effects. Therefore, an important goal is to identify small molecules that interact with previously identified molecular pathways (i.e. TGF-β signaling) involved in the development of fibrosis to prevent the progression or reverse the fibrosis seen in patients.

### Wnt Signaling and Human Disease

Vertebrate Wnt proteins are homologues of the Drosophila wingless gene and have been show to play important roles in regulating cell differentiation, proliferation, and polarity. Cadijan, K M. et al., Genes Dev. 11:3286-3305 (1997); Parr, B. A. et al., Curr. Opin. Genet. Dev. 4:523-528 (1994); Smalley, M. J. et al., Cancer Met. Rev. 18:215-230 (1999); and Willert K I et al., Curr. Opin. Genet. Dev. 8:95-102 (1998). Wnt proteins are cysteine-rich secreted glycoproteins that signal through at least three known pathways. The best understood of these, commonly called the canonical pathway, involves binding of Wnt proteins to frizzled cell surface receptors and low-density lipoprotein cell surface co-receptors, thereby inhibiting glycogen synthase kinase 3β (GSK-3β) phosphorylation of the cytoskeletal protein β-catenin. This hypophospborylated β-catenin is then translocated to the nucleus, where it binds to members of the LEF/TCF family of transcription factors. Binding of β-catenin converts LEF/TCF factors from repressors to activators, thereby switching on cell-specific gene transcription. The other two pathways that Wnt proteins can signal through either activate calmodulin kinase H and protein kinase C (known as the Wnt/Ca++pathway) or jun N-terminal kinase (also known as the planar cell polarity pathway).

Several components of the Wnt pathway have been implicated in tumorigenesis in humans and mice, and studies of those have in turn identified a role for β-catenin. Wnt1 was first identified from a retroviral integration in mice that caused mammary tumors. Tsukamoto, A. S. et al., Cell 55:619-625 (1988); and Jue, S. F. et al., Mol. Cell. Biol. 12:321-328 (1992). Overexpression of protein kinase CK2 in the mammary gland, which potentiates β-catenin-dependent Wnt signaling, also increases the incidence of mammary tumors in transgenic mice. Landesman-Bollag, E. et al., Oncogene 20:3247-3257 (2001); and Song, D. H. et al., J. Biol. Chem. 275:23790-23797 (2000). Gut epithelia has revealed the most extensive correlation between Wnt signaling and tumorigenesis. Several reports have described mutations in β-catenin itself in some colon tumors and these mutations occur in or near the GSK-3β phosphorylation sites. Polakis, P. et al., Adv. Exp. Med. Biol. 470:23-32 (1999); and Morin, P. J. et al., Science 275:1787-1790 (1997). Chilosi and colleagues (Chilosi, M. et al., Am. J. Pathol. 162:1497-1502, 2003) investigated β-catenin mutations in IPF patients but did not identify any. This is consistent with a mechanism in which the aberrant activation of the Wnt pathway is a response and not a cause of IPF.

### Lung Development and Wnt Signaling

In the mouse, the lung arises from the primitive foregut endoderm starting at approximately E9.5 during mouse development (Warburton, D. et al., Mech. Dev. 92:55-81, 2000.) This primitive epithelium is surrounded by mesodermally derived multipotent mesenchymal cells, which in time will differentiate into several cell lineages including bronchial and vascular smooth muscle, pulmonary fibroblasts, and endothelial cells of the vasculature. During gestation, the airway epithelium evolves and grows through a process termed branching morphogenesis. This process results in the three-dimensional arborized network of airways required to generate sufficient surface area for postnatal respiration. Mouse embryonic lung development can be divided into at least four stages: embryonic (E9.5 to E12.5), pseudoglandular (E12.5 to E16.0), canalicular (E16.0 to E17.5), and saccular/alveolar (E17.5 to postnatal).

During development, epithelial-mesenchymal signaling plays an important role in the regulation of both epithelial and mesenchymal cell differentiation and development. Several important signaling molecules are expressed in the airway epithelium and signal to the adjacent mesenchyme including members of the bone morphogenetic family (BMP-4), transforming growth factor family (TGF-β1, -2), and sonic hedgehog (SHH). In turn, the mesenchyme expresses several signaling molecules such as FGF-7, -9, and -10, important for lung epithelial development and proliferation. Gain of function and loss of function experiments in mice have demonstrated an important role for each of these factors in regulating lung epithelial and mesenchymal proliferation and differentiation. Bellusci, S., et al., Development 1997, 124:4867-4878; Simonet, W. S., et al., Proc. Natl. Acad. Sci. USA 1995, 92:12461-12465; Clark, J. C., et al., Am. J. Physiol. 2001, 280:L705-L715; Min, H., et al., Genes Dev. 1998, 12:3156-3161; Motoyama, J., et al., Nat. Genet. 1998, 20:54-57; Litingtung, Y., et al., Nat. Genet. 1998, 20:58-61; Pepicelli, C. V., et al., Curr. Biol. 1998, 8:1083-1086; Weaver, M., et al., Development 1999, 126:4005-4015.

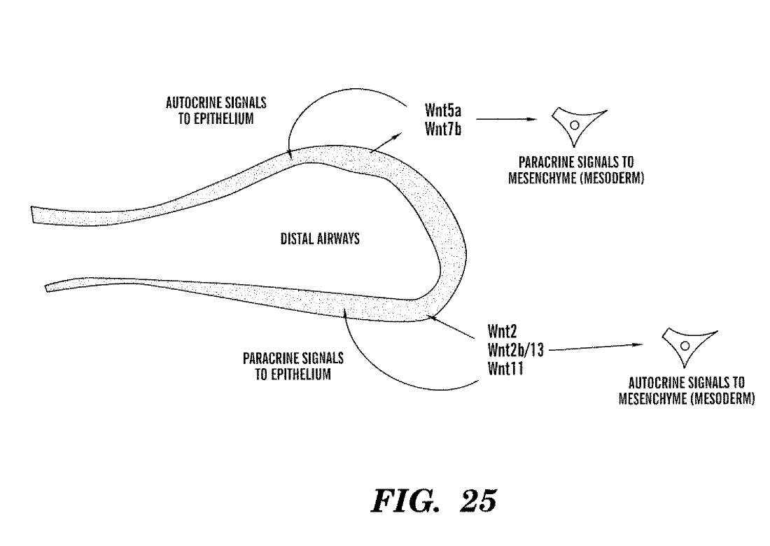

Wnt signaling also plays a role during lung development Several Wnt genes are expressed in the developing and adult lung including Wnt2, Wnt2b/13, Wnt7b, Wnt5a, and Wnt11. Kispert, A., et al., Development 1996, 122:3627-3637; Lin, Y., et al., Dev. Dyn. 2001, 222:26-39; Monkley, S. J., et al., Development 1996, 122:3343-3353; Yamaguchi, T. P., et al., Development 1999, 126:1211-1223; Weidenfeld, J., et al., J. Biol. Chem. 2002, 277:21061-21070. Of these, Wnt5a and Wnt7b are expressed at high levels exclusively in the developing airway epithelium during lung development. Wnt2, Wnt5a, and Wnt7b have been inactivated through homologous recombination in mice. Wnt2-null mice do not display an overt lung phenotype and Wnt5a null mice have late-stage lung maturation defects, corresponding to expression of Wnt5a later in lung development. (Monkley, (1996); Li, C. et al., Dev. Biol. 248:68-81 (2002)). Inactivation of Wnt7b results in either early embryo demise because of defects in extra-embryonic tissues or perinatal demise because of defects in lung development. Parr, B. A., et al., Dev. Biol. 237:324-332 (2001); Shu, W. et al., Development 129:4831-4842 (2002). These lung defects include decreased mesenchymal proliferation, lung hypoplasia caused by reduced branching, and pulmonary vascular smooth muscle defects leading to blood vessel hemorrhage in the lung. (Shu, W. (2002)). Thus, Wnt signaling regulates important aspects of both epithelial and mesenchymal development during gestation, likely through both autocrine and paracrine signaling mechanisms. (FIG. 25.)

Accumulation of nuclear β-catenin in has been observed in both epithelial and mesenchymal (myofibroblasts) cell lineages in adult human lung. Other reports support these observations during mouse lung development. (Tebar, R., et al., Mech. Dev. 109:437-440 (2001)). Type 2 pneumocytes appear to express high levels of β-catenin both in the embryo and in the adult (Tebar, 2001). Type 2 cells are precursors of type 1 cells, which form the thin diffusible stratum important for gas exchange in the lung. Type 2 cells have been shown to re-enter the cell cycle, grow, and differentiate into type 1 cells in some models of lung re-epithelialization. (Borok, Z. et al., Am. J. Respir. Cell Mol. Biol. 12:50-55 (1995); Danto, S. I. et al., Am. J. Respir. Cell Mol. Biol. 12:497-502 (1995)).

Importantly, type 2 cells proliferate excessively during idiopathic fibrosis (IPF) and other proliferative lung diseases, and increased nuclear β-catenin in these cells suggests that Wnt signaling regulates this proliferation. (Kawanami, O., et al., Lab. Invest. 46:39-53 (1982); Kasper, M. et al., Histol. Histopathol. 11:463-483 (1996)). Increased proliferation of type 2 cells in IPF may also inhibit their differentiation into type 1 cells because excessive proliferation is often antagonistic to cellular differentiation. In this context, it is important to note that expression of certain important transcriptional and signaling regulators in the lung decreases with gestational age. Forced overexpression of some of these such as BMP-4, GATA6, and Foxa2 results in aberrant lung development that exhibits many aspects of arrested lung epithelial maturity. (Weaver, 1999; Koutsourakis, M. et al., Mech. Dev. 105:105-114, 2001; Zhou, L. et al., Dev. Dyn. 210:305-314, 1997). Thus, a careful balance of the correct spatial and temporal expression of certain regulatory genes is required for normal lung development, and improper activation of these pathways can result in severe defects in epithelial differentiation.

Nuclear β-catenin is found in the mesenchyme adjacent to the airway epithelium (Chilosi, 2003), and this is significant especially because these cells appear to be myofibroblastic in nature and may contribute to bronchial and vascular smooth muscle in the lung. Although Wnt signals in these mesenchymal cells could be autocrine in nature, it is just as likely that the mesenchymal cells are responding to a paracrine signal from the airway epithelium where Wnts such as Wnt5a and Wnt7b are expressed. In this way, the epithelium may be responsible for causing the aberrant activation of Wnt signaling in adjacent mesenchyme, leading to increased fibrosis and damage to the lung. This is particularly relevant because of the increase in the number of type 2 cells in the airways of IPF patients. This may also be reflective of a switch to an embryonic phenotype in the alveolus, where type 1 cells are rare. In turn, this would result in an increase in expression of several genes, including Wnts such as Wnt7b, whose expression is dramatically down-regulated in postnatal development. (Weidenfeld, 2002; Shu, 2002.) The increased level of Wnts may inhibit the proper differentiation of more mature alveolar cells such as type 1 cells, impairing the repair process.

Because nuclear translocation of β-catenin is a result of Wnt signaling activity, its presence in cells such as distal airway epithelium and in mesenchyme adjacent to airway epithelium suggests that epithelial-mesenchymal Wnt signaling is active and likely plays an important role during both lung development and disease states such as IPF.

### Regulation of Cell-Matrix Interactions by Wnt Signaling

A link has been shown between Wnt signaling and regulation of cell-matrix interactions including cell adhesion and migration. In particular, Wnt signaling has been shown to affect cell motility and invasiveness of melanoma cells. (Weer ara+na, A. T. et al., Cancer Cell 1:279-288 (2002.) In this system, melanoma cells overexpressing Wnta displayed increased adhesiveness, which correlated to a reorganized actin cytoskeleton. (Weer, 2002.) These data suggest that Wnt5a expression correlates directly with the metastatic ability of melanoma tumors.

In IPF lung tissue (Chilosi, 2003), the important extracellular matrix metalloproteinase matrilysin was overexpressed in some of the cells containing high levels of nuclear β-catenin. This is supported by previous studies showing that matrilysin is a molecular target of Wnt signaling. (Crawford, H. C., Oncogene 18:2883-2891, 1999.) Matrilysin has been linked to a role in carcinogenesis both in intestinal and endometrial tumors. Increased matrilysin expression strongly correlates with increased nuclear β-catenin expression and inhibition of this nuclear translocation results in decreased matrilysin expression. (Crawford, 1999.) Without being bound by a specific hypothesis, the mechanism may involve increased degradation of the extracellular matrix from increased matrilysin expression, leading to decreased cell adhesion and increased cell motility. In IPF, this might reduce the ability of both epithelial and mesenchymal cells to properly restructure the alveolar architecture after injury. In addition, extracellular matrix integrity may be required for type 1 cell differentiation, because of their flattened morphology and the very large surface area that they cover in the alveolus. This process may contribute to an increase in type 2 cell proliferation, which in turn could decrease type 1 cell differentiation.

### Wnt Signaling and IPF

Without being bound by a specific hypothesis, several models could explain the finding that Wnt signaling is aberrantly activated in IPF. First, unregulated activation of the Wnt signaling pathway could be a physiological response to either lung injury or the repair process, possibly because of the requirement of the Wnt pathway for proliferation in cells such as type 2 alveolar epithelium and adjoining myofibroblasts. In this model, Wnt signaling should deactivate once the repair process is complete, leading to a return to normal proliferation. In the second model, aberrant Wnt signaling is the initiating event leading to increased cell proliferation in type 2 cells, which may inhibit their ability to differentiate into type 1 cells and restructure the alveolar architecture properly. Either injury-induced or spontaneous mutations in certain components of the canonical Wat pathway or in regulatory molecules that regulate this pathway may result in this dysregulation of cell proliferation. The fact that nuclear β-catenin is up-regulated in other lung proliferative diseases suggests that the previous data (Chilosi, 2003) may be a response and not a primary causative event in IPF. Moreover, the unregulated proliferation in type 2 cells and mesenchymal fibroblasts along with the increased presence of nuclear β-catenin suggests that the Wnt pathway is continuously stimulated in lung diseases such as IPF and that inhibitors of Wnt signaling may provide a means to control this proliferation.

Increased nuclear β-catenin was detected in the mesenchyme adjacent to the airway epithelium, describes as myofibroblasts. (Chilosi, 2003.) These myofibroblasts can induce apoptosis in neighboring epithelial cells in vitro and in vivo, probably through degradation of the extracellular matrix. (Uhal, B. D. et al., Am. Physiol. 275:L1192-L1199, 1998; Uhal, B. D. et al., Am. J. Physiol. 269:L819-L822, 1995; Selman, M. et al., Am. J. Physiol. 279:L562-L574, 2000.) In addition, in IPF there appears to be either a lack of re-epithelialization or an increase in type 2 cells with little if any maturation of type 1 cells, leading to injured areas with exposed mesodermal components or re-epithelialized with immature type 2 cells. Since it has been demonstrated that type 2 cells express high levels of TGF-β1, which is a profibrotic cytokine, in IPF either scenario would inhibit the proper re-epithelialization of these injured areas, causing more fibrosis. (Kapanci, Y., et al., Am. J. Respir. Crit. Care Med. 152:2163-2169, 1995; Khalil, N., et al., Am. J. Respir. Cell Mol. Biol. 5:155-162, 1991.) This process could go unchecked and eventually lead to massive changes in tissue architecture, eventual tissue destruction, and loss of lung function.

Connective tissue growth factor (CTGF) is a 36 to 38 kD cysteine-rich peptide containing 349 amino acids. It belongs to the CCN (CTGF, cyr 61/cef 10, nov) family of growth factors. The gene for CTGF was originally cloned from a human umbilical endothelial cell cDNA library. CTGF has been detected in endothelial cells, fibroblasts, cartilaginous cells, smooth muscle cells, and some cancer cell lines. Earlier studies revealed that TGF-β1 increases CTGF mRNA markedly in human foreskin fibroblasts. PDGF, EGF, and FGF were also shown to induce CTGF expression, but their effects were only transient and weak.

Connective tissue growth factor has diverse bioactivities. Depending on cell types, CTGF was shown to trigger mitogenesis, chemotaxis, ECM production, apoptosis, and angiogenesis. In earlier studies, CTGF was noted to have mitogenic and chemotactic effects on fibroblasts. CTGF was also reported to enhance the mRNA expression of α1 (I) collagen, fibronectin, and α5 integrin in fibroblasts. The finding that TGF-β increases CTGF synthesis and that TGF-β and CTGF share many functions is consistent with the hypothesis that CTGF is a downstream mediator of TGF-β.

The mechanism by which CTGF exerts its effects on cells, especially its signal transduction, is still unclear. CTGF was reported to bind to the surface of fibroblasts with high affinity, and this binding was competed with recombinant PDGF BB. This suggests that CTGF binds to a certain class of PDGF receptors, or that there is some cross reactivity of PDGF BB with CTGF receptors.

Connective tissue growth factor mRNA has been detected in fibroblasts of sclerotic lesions of patients with systemic sclerosis. In patients with localized scleroderma, CTGF mRNA was detected in fibroblasts in tissues from sclerotic stage more than the inflammatory stage, which suggests a close correlation between CTGF and fibrosis. Similar results were also obtained in keloid and other fibrotic diseases. Subsequently, expression of CTGF has been reported in a variety of fibrosis, such as liver fibrosis, pulmonary fibrosis, and heart fibrosis. CTGF is also implicated in dermal fibrosis of scleroderma. However, the detailed role of CTGF in fibrosis is still unclear. Further studies are needed to clarify this point.

The CCN family comprises cysteine-rich 61 (CYR61/CCN1), connective tissue growth factor (CTGF/CCN2), nephroblastoma overexpressed (NOV/CCN3), and Wnt-induced secreted proteins-1 (WISP-1/CCN4), -2 (WISP-2/CCN5) and -3 (WISP-3/CCN6). These proteins stimulate mitosis, adhesion, apoptosis, extracellular matrix production, growth arrest and migration of multiple cell types. Many of these activities probably occur through the ability of CCN proteins to bind and activate cell surface integrins.

Connective tissue growth factor (CTGF) has been identified as a potential target of Wnt and BMP signaling. It has been confirmed by microarray results, and demonstrated that CTGF was up-regulated at the early stage of BMP-9 and Wnt3A stimulations and that Wnt3A-regulated CTGF expression was beta-catenin-dependent.

The synthesis and identification of conformationally constrained α-helix mimetics and their application to diseases are discussed in (Walensky, L. D. et al Science 305, 1466, 2004; Klein, C. Br. J. Cancer. 91, 1415, 2004).

The present invention is directed to conformationally constrained compounds which mimic the secondary structure of α-helix regions of biological peptide and proteins (also referred to herein as “α-helix mimetics” and chemical libraries relating thereto. The α-helix mimetic structures of the present invention will be useful as bioactive agents, such as diagnostic, prophylactic, and therapeutic agents.

The α-helix mimetic structures of the present invention are useful as bioactive agents, including (but not limited to) use as diagnostic, prophylactic and/or therapeutic agents. The α-helix mimetic structure libraries of this invention are useful in the identification of such bioactive agents. In the practice of the present invention, the libraries may contain from tens to hundreds to thousands (or greater) of individual α-helix structures (also referred to herein as “members”).

In one aspect of the present invention, a o-helix mimetic structure is disclosed having the following formula (I):

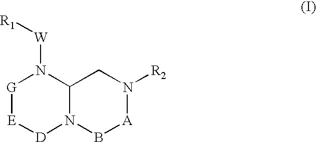

Wherein A is —(C═O)—CHR₃—, or —(C═O), B is N—R₅— or —CHR₆—, D is —(C═O)—(CHR₇)— or —(C═O)—, E is -(ZR₈)— or (C═O), G is —(XR₉)ₙ—, —(CHR₁₀)—(NR₆), —(C—O)—(XR₁₂)—, —(C═N—W—R₁)—, —(C═O)—, X—(C═O)—R₁₃, X—(C—O)—NR₁₃R₁₄, X—(SO₂)—R₁₃, or X—(C═O)—OR₁₃, W is —Y(C═O)—, —(C═O)NH—, —(SO₂)—, —CHR₁₄, (C—O)—(NR₁₅)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, or nothing, Y is oxygen or sulfur, X and Z is independently nitrogen or CH, n=0 or 1; and R₁, R₂, R₃, R₄, R₅, R₆, R₇, R₈, R₉ R₁₀, R₁₁, R₁₂, R₁₃, R₁₄, and R₁₅ are the same or different and independently selected from an amino acid side chain moiety or derivative thereof, the remainder of the molecule, a linker and a solid support, and stereoisomers thereof.

More specifically, R₁, R₂, R₃, R₄, R₅, R₆, R₇, R₈, R₉ R₁₀, R₁₁, R₁₂, R₁₃, R₁₄, and R₁₅ are independently selected from the group consisting of aminoC₂₋₅alkyl, guanidineC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidino C₂₋₅alkyl, diC₁₋₄alkylamidinoC₂₋₅alkyl, C₁₋₃alkoxy, Phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₃alkyl, nitro, carboxy, cyano, sulfuryl or hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), bisphenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyridyl, substituted pyridyl, (where the substituents are independently selected from one or more of amino, amidino, guanidino, hydrazino, amidrazonyl, C₁₋₄alkylamino, C₁₋₄dialkylamino, halogen, perfluoro C₁₋₄alkyl C₁₋₄alkyl, C₁₋₃alkoxy, nitro, carboxy, cyano, sulfuryl or hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl or hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl or methyl), imidazolinylCalkyl N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl and 4-aminocyclohexylC₀₋₂alkyl.

In one embodiment, R₁, R₂, R₆ of E, and R₇, R₈ and R₉ of G are the same or different and represent the remainder of the compound, and R₃ or A, R₄ of B or R₅ of D is selected from an amino acid side chain moiety or derivative thereof. As used herein, the term “remainder of the compound” means any moiety, agent, compound, support, molecule, linker, amino acid, peptide or protein covalently attached to the α-helix mimetic structure at R₁, R₂, R₅, R₆, R₇, R₈ and/or R₉ positions. This term also includes amino acid side chain moieties and derivatives thereof.

In another embodiment, where B is CHR₆ and W is —Y(C═O)—, —(C═O)NH—, —(SO₂)—, —CHR₁₄, or (C═O)—(NR₁₅)—, G cannot be CHR₉, NR₉, (C═O)—CHR₁₂, (C═O)—NR₁₂, or no atom at all.

As used herein, the term “amino acid side chain moiety” represents any amino acid side chain moiety present in naturally occurring proteins including (but not limited to) the naturally occurring amino acid side chain moieties identified in Table 1. Other naturally occurring amino acid side chain moieties of this invention include (but are not limited to) the side chain moieties of 3,5-dibromotyrosine, 3,5-diiodotyrosine, hydroxylysine, γ-carboxyglutamate, phosphotyrosine and phosphoserine. In addition, glycosylated amino acid side chains may also be used in the practice of this invention, including (but not limited to) glycosylated threonine, serine and asparagine.

TABLE 1
Amino Acid Side Chain Moieties

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| | Histidine |
| —CH₂COO⁻ | Aspartic acid |
| —CH₂CH₂COO⁻ | Glutamic acid |
| —CH₂CONH₂ | Asparagine |
| —CH₂CH₂CONH₂ | Glutamine |
| | Phenylalanine |
| | Tyrosine |
| | Tryptophan |
| —CH₂SH | Cysteine |
| —CH₂CH₂SCH₃ | Methionine |
| —CH₂OH | Serine |
| —CH(OH)CH₃ | Threonine |
| | Proline |
| | Hydroxyproline |

In addition to naturally occurring amino acid side chain moieties, the amino acid side chain moieties of the present invention also include various derivatives thereof. As used herein, a “derivative” of an amino acid side chain moiety includes modifications and/or variations to naturally occurring amino acid side chain moieties. For example, the amino acid side chain moieties of alanine, valine, leucine, isoleucine and phenylalanine may generally be classified as lower chain alkyl, aryl, or arylalkyl moieties. Derivatives of amino acid side chain moieties include other straight chain or brached, cyclic or noncyclic, substitutes or unsubstituted, saturated or unsaturated lower chain alkyl, aryl or arylalkyl moieties.

As used herein, “lower chain alkyl moieties” contain from 1-12 carbon atoms, “lower chain aryl moieties” contain from 6-12 carbon atoms and “lower chain aralkyl moieties” contain from 7-12 carbon atoms. Thus, in one embodiment, the amino acid side chain derivative is selected from a C₁₋₁₂ alkyl, a C₆₋₁₂ aryl and a C₇₋₁₂ arylalkyl, and in a more preferred embodiment, from a C₁₋₇ alkyl, a C₆₋₁₀ aryl and a C₇₋₁₁ arylalkyl.

Amino side chain derivatives of this invention further include substituted derivatives of lower chain alkyl, aryl, and arylalkyl moieties, wherein the substituents is selected from (but are not limited to) one or more of the following chemical moieties: —OH, —OR, —COOH, —COOR, —CONH₂, —NH₂, —NHR, —NRR, —SH, —SR, —SO₂R, —SO₂H, —SOR and halogen (including F, Cl, Br and I), wherein each occurrence of R is independently selected from straight chain or branched, cyclic or noncyclic, substituted or unsubstituted, saturated or unsaturated lower chain alkyl, aryl, and aralkyl moieties. Moreover, cyclic lower chain alkyl, aryl and arylalkyl moieties of this invention include naphthalene, as well as heterocyclic compounds such as thiophene, pyrrole, furan, imidazole, oxazole, thiazole, pyrazole, 3-pyrroline, pyrrolidine, pyridine, pyrimidine, purine, quinoline, isoquinoline and carbazole. Amino acid side chain derivatives further include heteroalkyl derivatives of the alkyl portion of the lower chain alkyl and aralkyl moieties, including (but not limited to) alkyl and aralkyl phosphonates and silanes.

Representative R₁, R₂, R₅, R₆, R₇, R₈ and R₉ moieties specifically include (but are not limited to) —OH, —OR, —COR, —COOR, —CONH₂, —CONR, —CONRR, —NH₂, —NHR, —NRR, —SO₂R and —COSR, wherein each occurrence of R is as defined above.

In a further embodiment, and in addition to being an amino acid side chain moiety or derivative thereof (or the remainder of the compound in the case of R₁, R₂, R₅, R₆, R₇, R₈ and R₉), R₁, R₂, R₅, R₆, R₇, R₈ or R₉ may be a linker facilitating the linkage of the compound to another moiety or compound. For example, the compounds of this invention may be linked to one or more known compounds, such as biotin, for use in diagnostic or screening assay. Furthermore, R₁, R₂, R₅, R₆, R₇, R₈ or R₉ may be a linker joining the compound to a solid support (such as a support used in solid phase peptide synthesis) or alternatively, may be the support itself. In this embodiment, linkage to another moiety or compound, or to a solid support, is preferable at the R₁, R₂, R₇ or R₈ position, and more preferably at the R₁ or R₂ position.

In the embodiment wherein A is —(C═O)—CHR₃—, B is —N—R₄, D is —(C═O), E is -(ZR₆)—, G is —(C═O)—(XR₉)—, the α-helix mimetic compounds of this invention have the following general formula (III):

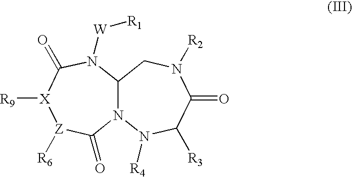

wherein R₁, R₂, R₄, R₆, R₇, R₈, W and X are as defined above, Y is —C═O, —(C═O)—O—, —(C═O)—NR₈, —SO₂—, or nothing, and Z is nitrogen or CH (when Z is CH, then X is nitrogen). In a preferred embodiment, R₁, R₂, R₆, R₇ and R₈ represent the remainder of the compound, and R₄ is selected from an amino acid side chain moiety. In a more specific embodiment wherein A is —O—CHR₃—, B is —NR₄—, D is —(C═O)—, E is -(ZR₆)—, Gi is (XR₇)ₙ—, the α-helix mimetic compounds of this invention have the following formula (IV):

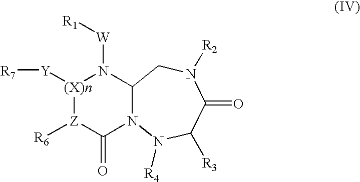

wherein R₁, R₂, R₄, R₆, R₇, W, X and n are as defined above, and Z is nitrogen or CH (when Z is nitrogen, then n is zero, and when Z is CH, then X is nitrogen and n is not zero). In a preferred embodiment, R₁, R₂, R₆, and R₇ represent the remainder of the compound, and R₄ is selected from an amino acid side chain moiety. In this case, R₆ or R₇ may be selected from an amino acid side chain moiety when Z and X are CH, respectively.

In the embodiment of structure (I) wherein A is C—(C═O), B is —(CHR₆)—, D is —(C═O)—, E is -(ZR₈)—, and G is —(NH)— or —(CH₂)—, and W is a substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, the α-helix mimetic compounds of this invention have the following general formula (V):

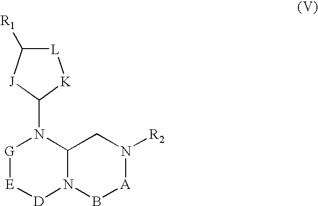

Wherein K is nitrogen, oxygen, or sulfur, L is nitrogen, oxygen, —(CH)—, or —(CH₂)—, J is nitrogen, oxygen, or sulfur, Z is nitrogen or CH, and R₁, R₂, R₆, R₈, and R₁₃ are selected from an amino acid side chain moiety.

Alternative embodiments of the invention relate to compounds having the general formula (VI):

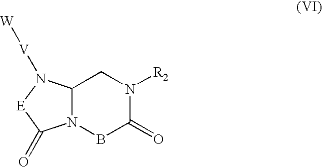

Wherein B is —(CHR₃)—, —(NR₃)—, E is —(CHR₄)—, V is —(XR₅)— or nothing, W is —(C═O)—(XR₆R₇), —(SO₂)—, substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, X is independently nitrogen, oxygen, or CH, and R₁, R₂, R₃, R₄, R₅, R₆, and R₇ are selected from an amino acid side chain moiety or derivative thereof, the remainder of the molecule, a linker and solid support, and stereoisomers thereof.

In the embodiments of formula (VI) wherein V is —(XR₅)— or nothing, and W is substituted or unsubstituted oxadiazole, substituted or unsubstituted triazole, substituted or unsubstituted thiadiazole, substituted or unsubstituted 4,5 dihydrooxazole, substituted or unsubstituted 4,5 dihydrothiazole, substituted or unsubstituted 4,5 dihydroimidizole, and X is independently introgen or CH, the compounds have the following general formula (VII):

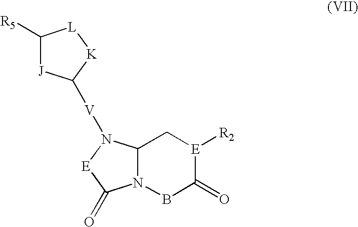

Wherein K is nitrogen, oxygen, or sulfur, L is nitrogen, oxygen, —(CH)—, or —(CH₂)—, J is nitrogen, oxygen, or sulfur, and R₂ and R₅ are defined as described above.

In preferred embodiments of the invention, R₂ in structures I through VII comprises an aromatic ring substituent such as a phenyl or naphthyl group that is substituted with a basic moiety such a primary or secondary amine. The aromatic ring substituent may also be a heterocycle, such as a purine or indole. Some embodiments of the invention also provide for aromatic ring substituents that may be substituted with one or two halogen moieties.

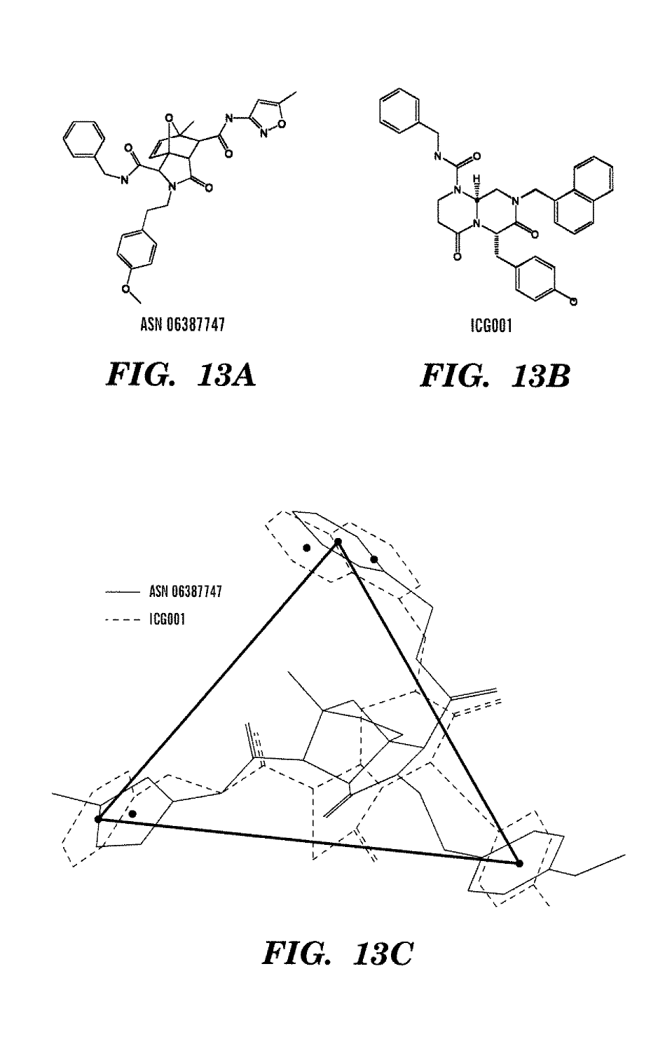

A feature of many α-helix mimetic compounds is that they provide a scaffolding that places three hydrophobic functional groups, which may also be referred to as pharmacophore rings, in a specific, spatially-defined orientation referred to as an “optimized chemical space”. The optimized chemical space may be triangular, with the centers of three functional groups forming the three points of the triangle. An example of an optimized chemical space is one in which the lengths of the three sides of the triangle are around 9.6±0.5 Angstroms (symbolized hereafter by “Å”), 9.2±0.5 Å, and 10.3±0.5 Å. FIG. 13C depicts two superimposed structures having three such pharmacophore rings forming a triangle in space. A number of different compounds exhibit such an optimized chemical space, and may be considered to be within the scope of the invention.

The compounds of general formula (I) of the present invention have one or more asymmetric carbons depending on the substituents. For example, where the compounds of general formula (I) contains one or more asymmetric carbons, two kinds of optical isomers exist when the number of asymmetric carbon is 1, and when the number of asymmetric carbon is 2, four kinds of optical isomers and two kinds of diastereomers exist. Pure stereoisomers including opticalisomers and diastereoisomers, any mixture, racemates and the like of stereoisomers all fall within the scope of the present invention. Mixtures such as racemates may sometimes be preferred from viewpoint of ease of manufacture.

When the compounds of general formula (I) of the present invention contains a basic functional group such as amino group, or when the compounds of general formula (I) of the present invention contains an aromatic ring which itself has properties of base (e.g., pyridine ring), the compound can be converted into a pharmaceutically acceptable salt (e.g., salt with inorganic acids such as hydrochloric acid and sulfuric acid, or salts with organic acids such as acetic acid and citric acid) by a known means. When the compounds of general formula (I) of the present invention contains an acidic functional group such as carboxyl group or phenolic hydroxyl group, the compound can be converted into pharmaceutically acceptable salt (e.g., inorganic salts with sodium, ammonia and the like, or organic salts with triethylamine and the like) by a known means. When the compounds of general formula (I) of the present invention contains a prodrugable functional group such as phenolic hydroxyl group, the compound can be converted into prodrug (eg., acetylate or phosphonate) by a known means. Any pharmaceutically acceptable salt and prodrug all fall within the scope of the present invention.

The various compounds disclosed by the present invention can be purified by known methods such as recrystallization, and variety of chromatography techniques (column chromatography, flash column chromatography, thin layer chromatography, high performance liquid chromatography).

The α-helix mimetic structures of the present invention may be prepared by utilizing appropriate starting component molecules (hereinafter referred to as “component pieces”). Briefly, in the synthesis of α-helix mimetic structures having formula (II), first and second component pieces are coupled to form a combined first-second intermediate, if necessary, third and/or fourth component pieces are coupled to form a combined third-fourth intermediate (or, if commercially available, a single third intermediate may be used), the combined first-second intermediate and third-fourth intermediate (or third intermediate) are then coupled to provide a first-second-third-fourth intermediate (or first-second-third intermediate) which is cyclized to yield the reverse-turn mimetic structures of this invention. Alternatively, the reverse-turn mimetic structures of formula (II) may be prepared by sequential coupling of the individual component pieces either stepwise in solution or by solid phase synthesis as commonly practiced in solid phase peptide synthesis.

Within the context of the present invention, a “first component piece” has the following formula S1

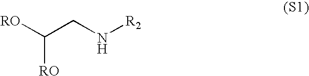

Wherein R₂ as defined above, and R is a protective group suitable for use in peptide synthesis. Suitable R groups include alkyl groups and, in a preferred embodiment, R is a methyl group. Such first component pieces may be readily synthesized by reductive amination or substitution reaction by displacement of H₂N—R₂ from CH(OR)₂—CHO or CH(OR)₂—H₂-Hal (wherein Hal means a halogen atom).

A “second component piece” of this invention has the following formula S2:

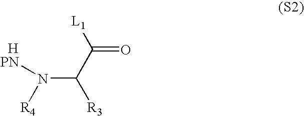

Where L₁ is carboxyl-activation group such as halogen atom, R₃, R₄ is as defined above, and P is an amino protective group suitable for use in peptide synthesis. Preferred protective groups include t-butyl dimethylsilyl (TBDMS), t-Butyloxycarbonyl (BOC), Methyloxycarbonyl (MOC), 9H-Fluorenylmethyloxycarbonyl (FMOC), and allyloxycarbonyl (Alloc). When L is —C(O)NHR, —NHR may be an carboxyl protective group. N-Protected amino acids are commercially available. For example, FMOC amino acids are available for a variety of sources. The conversion of these compounds to the second component pieces of this invention may be readily achieved by activation of the carboxylic acid group of the N-protected amino acid. Suitable activated carboxylic acid groups include acid halides where X is a halide such as chloride or bromide, acid anhydrides where X is an acyl group such as acetyl, reactive esters such as an N-hydroxysuccinimide esters and pentafluorophenyl esters, and other activated intermediates such as the active intermediate formed in a coupling reaction using a carbodiimide such as dicyclohexylcarbodiimide (DCC).

In the case of the azido derivative of an amino acid serving as the second component piece, such compounds may be prepared from the corresponding amino acid by the reaction disclosed by Zaloom et al. (J. Org. Chem. 46:5173-76, 1981).

A “third component piece” of this invention has the following formula S3:

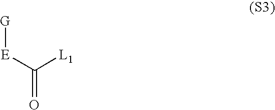

where G, E, and L₁ are as defined above. Suitable third component pieces are commercially available from a variety of sources or can be prepared by known methods in organic chemistry.

More specifically, the α-helix mimetic structures of this invention of formula (II) are synthesized by reacting a first component piece with a second component piece to yield a combined first-second intermediate, followed by either reacting the combined first-second intermediate with third component pieces sequentially to provide a combined first-second-third-fourth intermediate, and the cyclizing this intermediate to yield the α-helix mimetic structure.

The general synthesis of a α-helix having structure I′ may be synthesized by the following technique. A first component piece 1 is coupled with a second component piece 2 by using coupling reagent such as phosgene to yield, after N-deprotection, a combined first-second intermediate 1-2 as illustrated below:

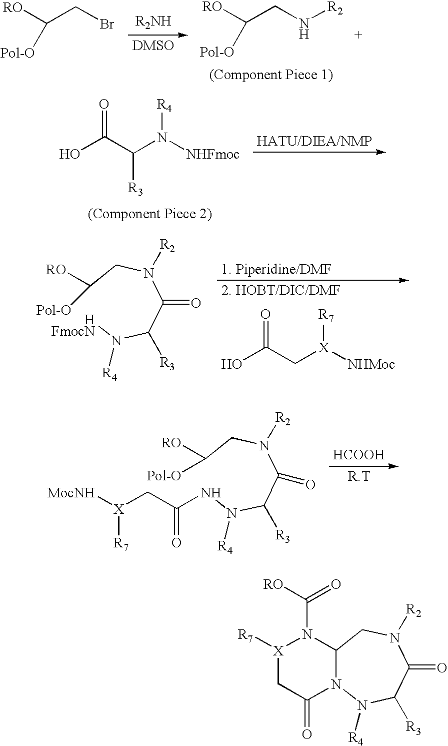

wherein R₁, R₂, R₄, R₇, Fmoc, Moc and X are as defined above, and Pol represents a polymeric support.

The α-helix mimetic structures of formula (II) and (IV) may be made by techniques analogous to the modular component synthesis disclosed above, but with appropriate modifications to the component pieces.

As mentioned above, the reverse-turn mimetics of U.S. Pat. No. 6,013,458 to Kahn, et al. are useful as bioactive agents, such as diagnostic, prophylactic, and therapeutic agents. The opiate receptor binding activity of representative reverse-turn mimetics is presented in Example 9 of said U.S. Pat. No. 6,013,458, wherein the reverse-turn mimetics of this invention were found to effectively inhibit the binding of a radiolabeled enkephalin derivative to the δ and μ opiate receptors, of which data demonstrates the utility of these reverse-turn mimetics as receptor agonists and as potential analgesic agents.

Therefore, since the compounds according to the present invention are of α-helix mimetic structures, they are useful for modulating cell signaling transcription factor-related peptides in a warm-blooded animal, comprising administering to the animal an effective amount of the compound of formula (I).

Further, the α-helix mimetic structures of the present invention may also be effective for inhibiting transcription factor/coactivator and transcription factor corepressor interactions.

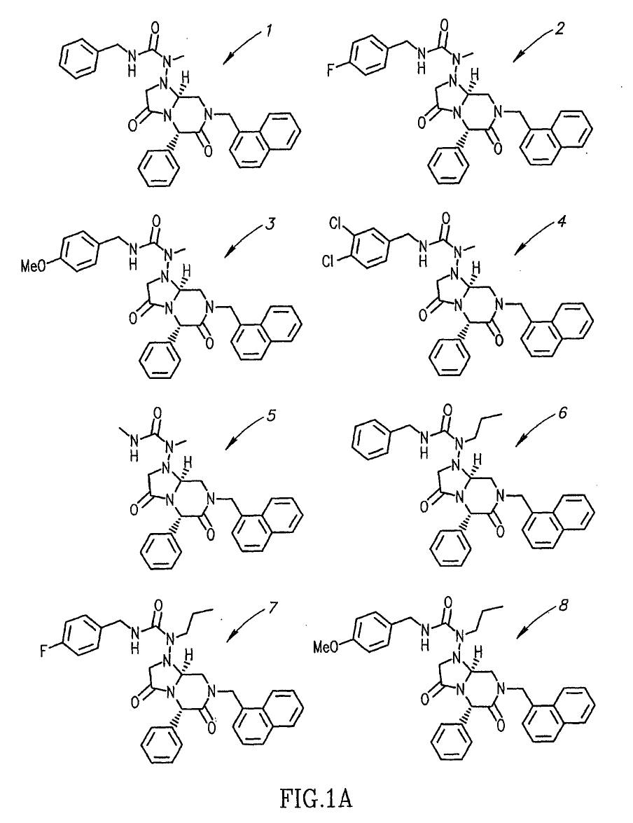

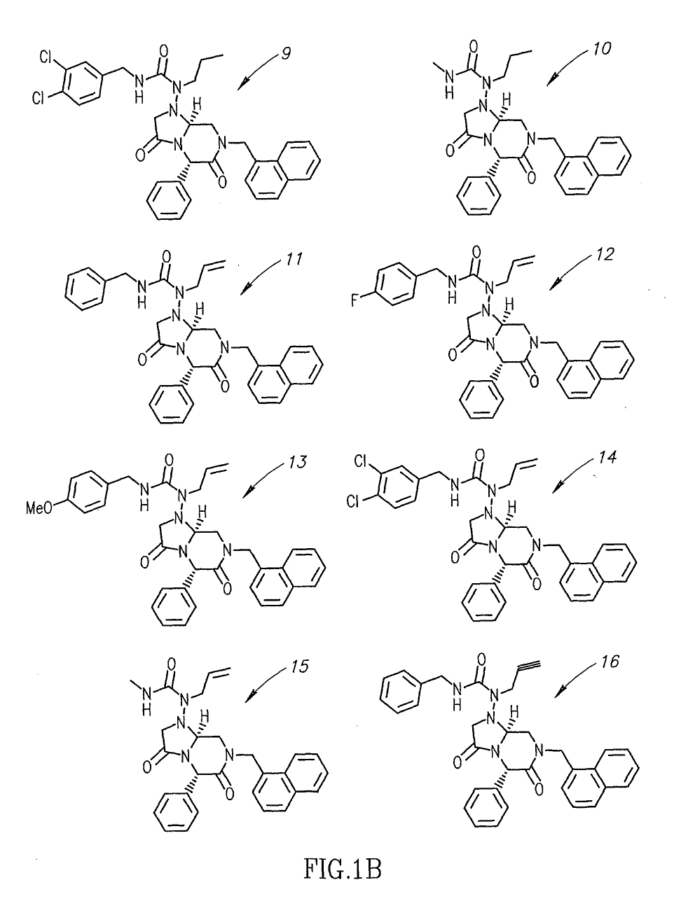

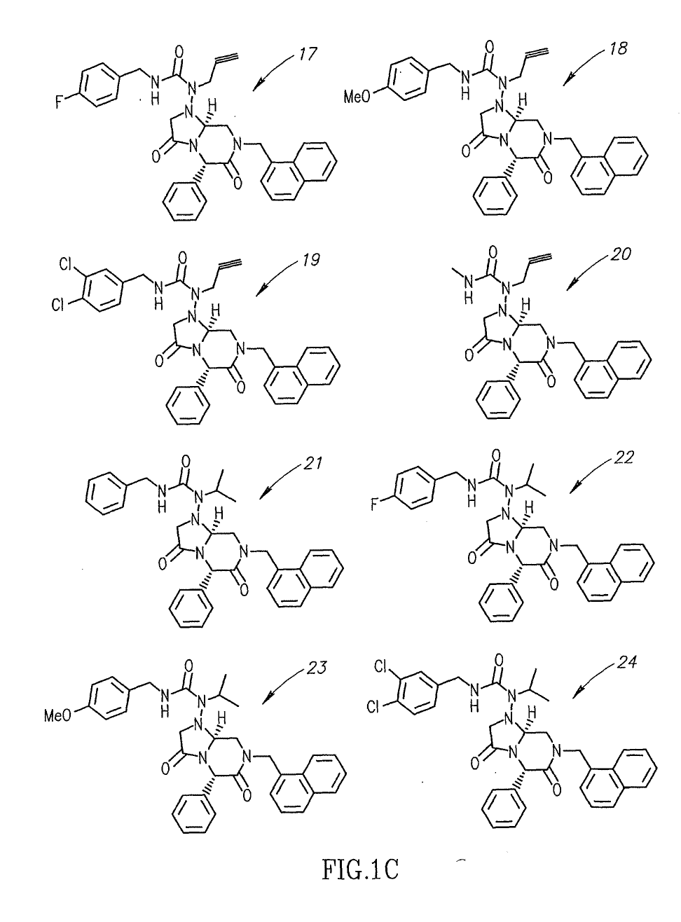

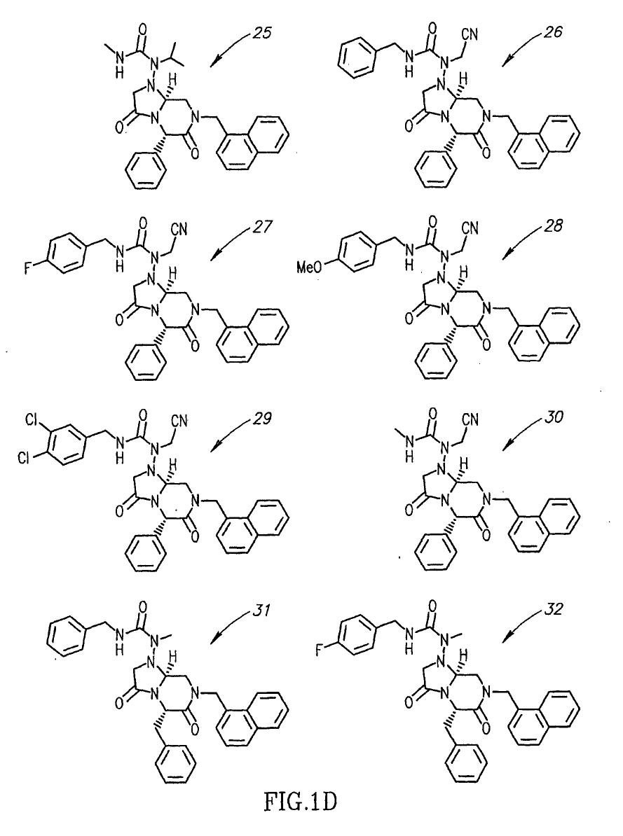

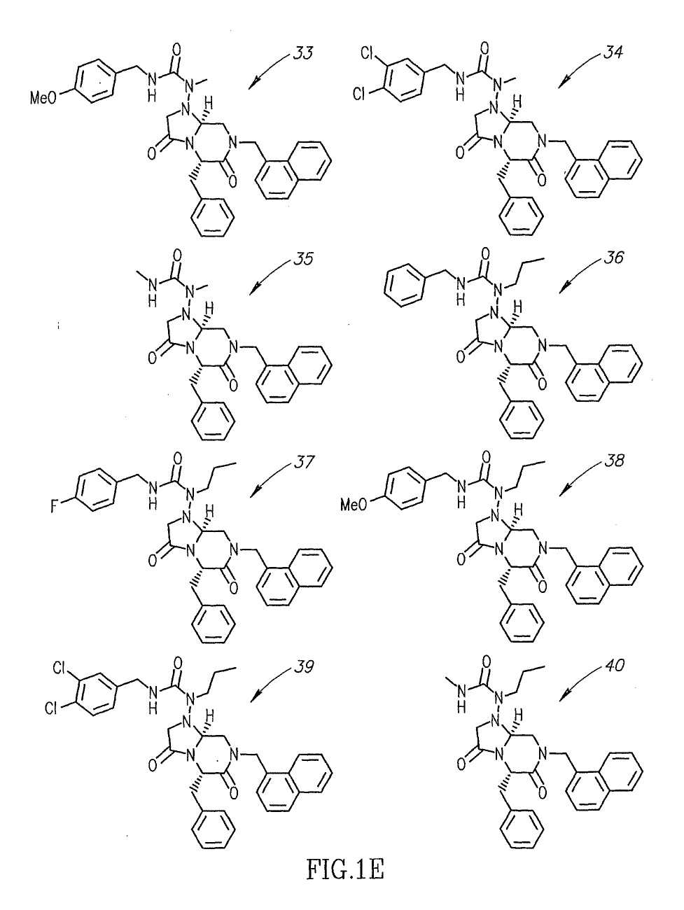

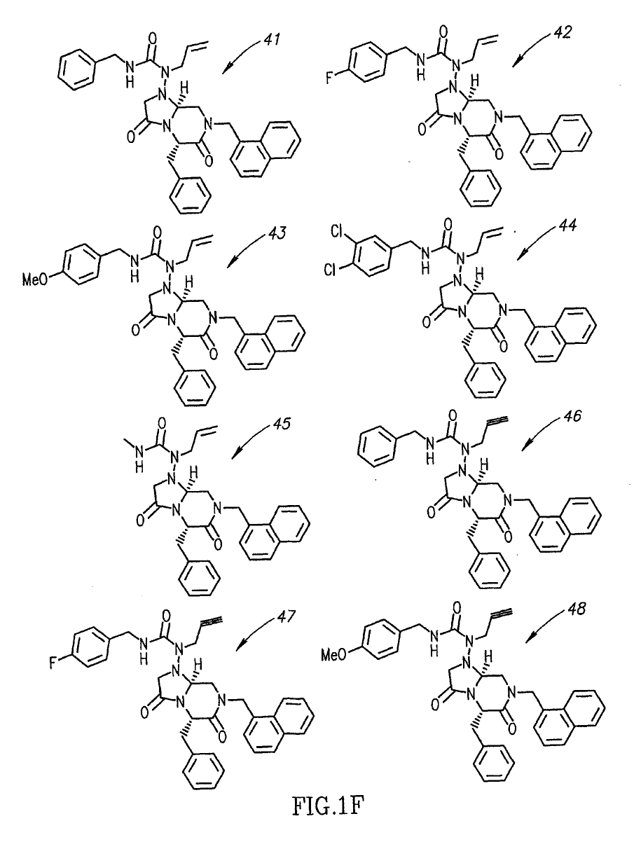

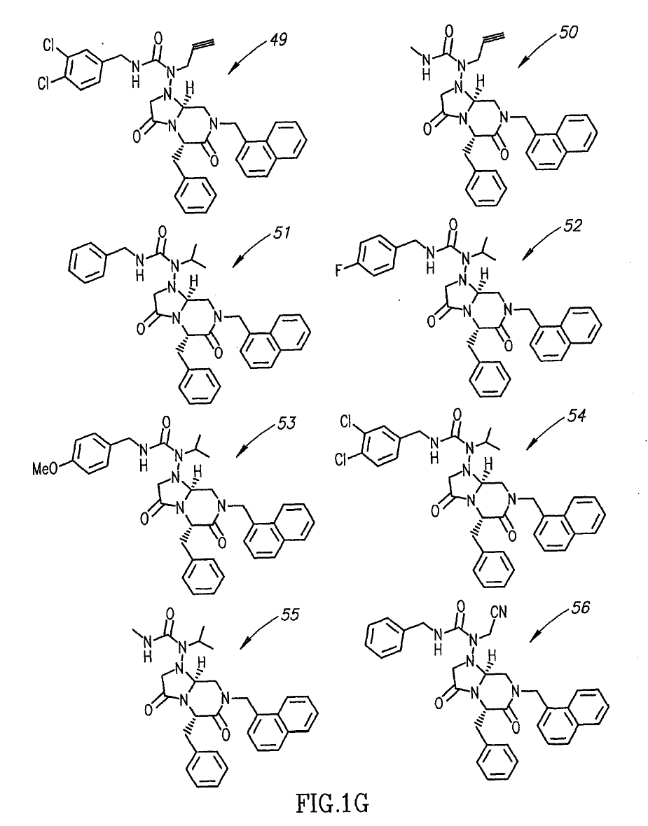

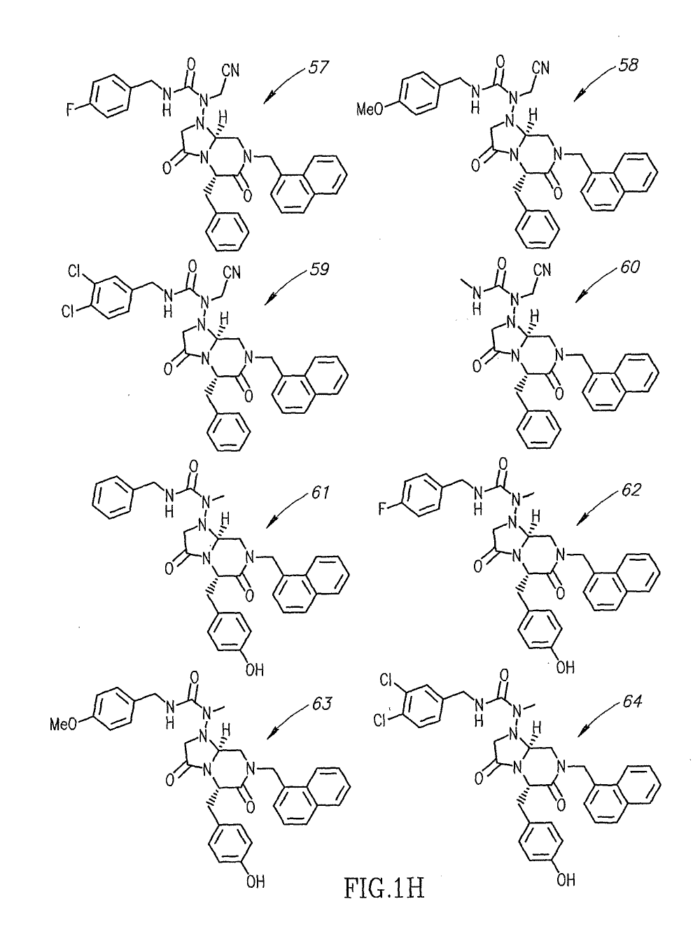

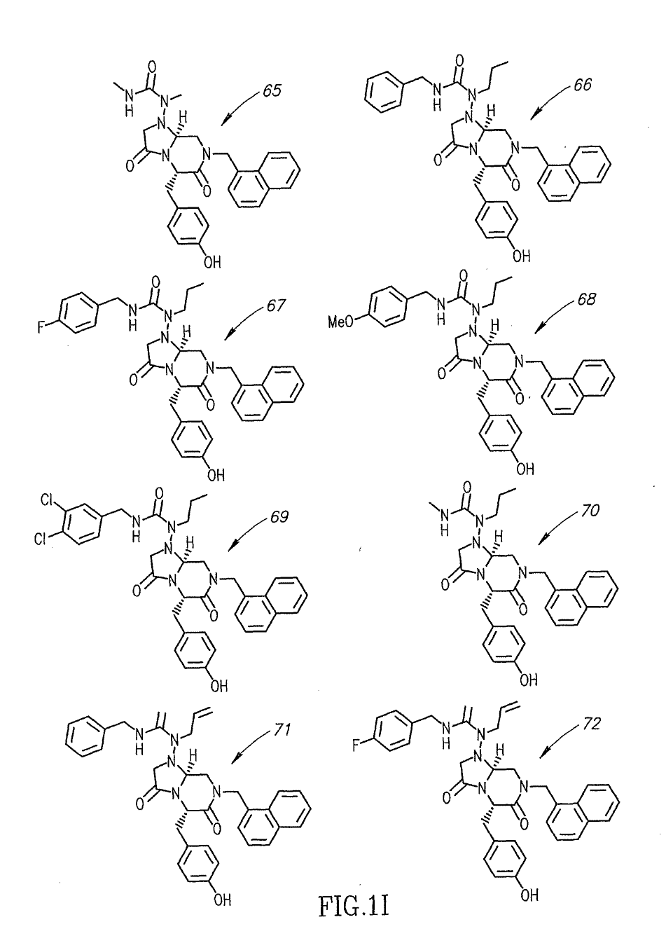

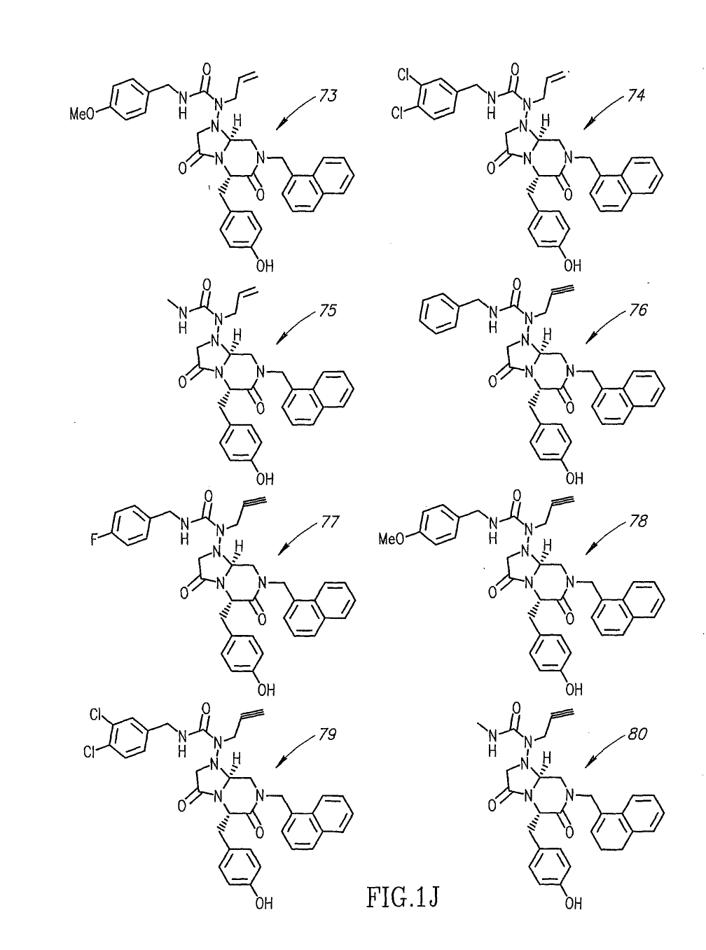

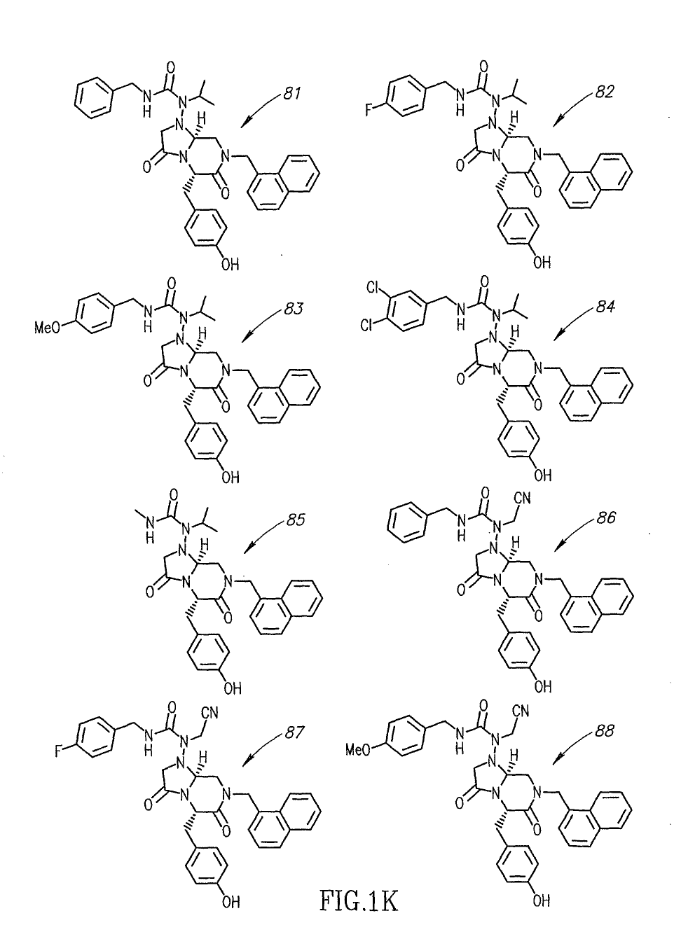

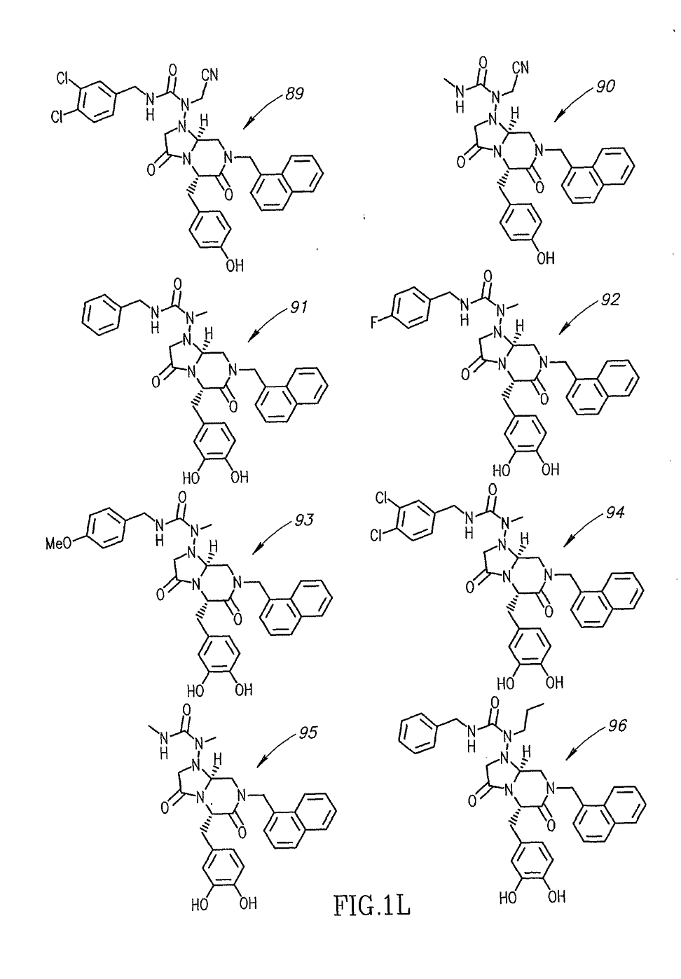

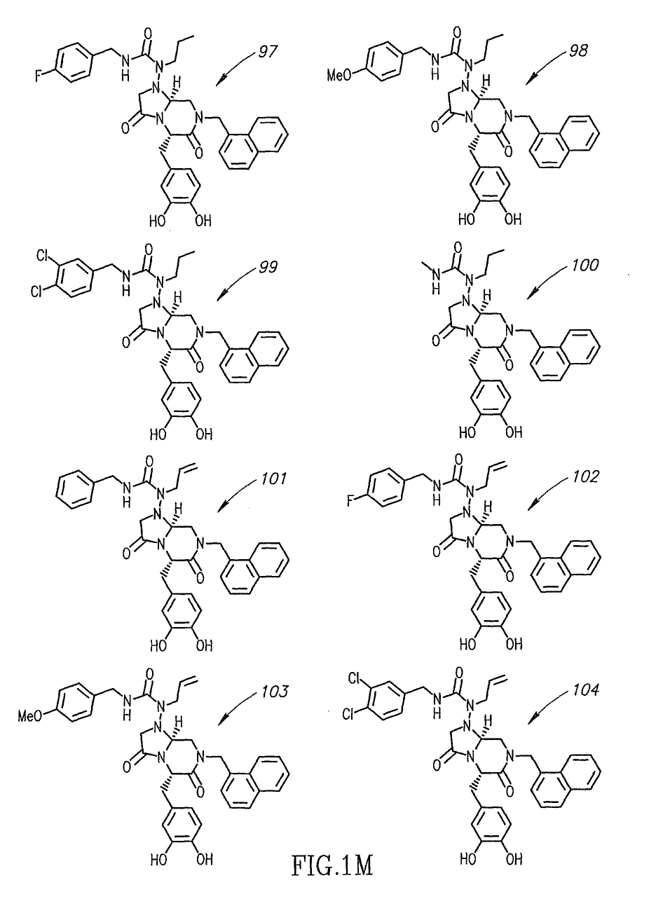

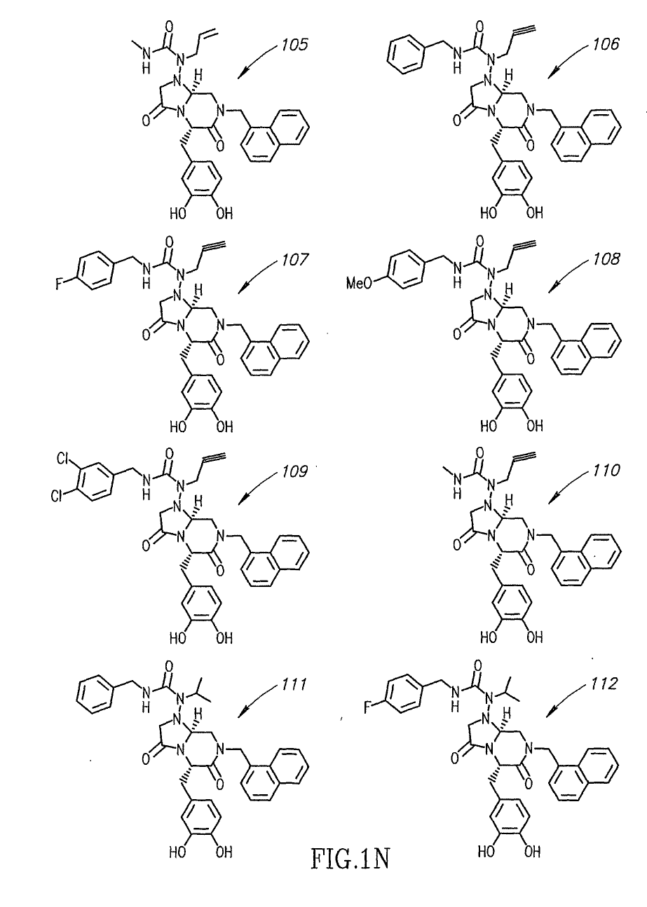

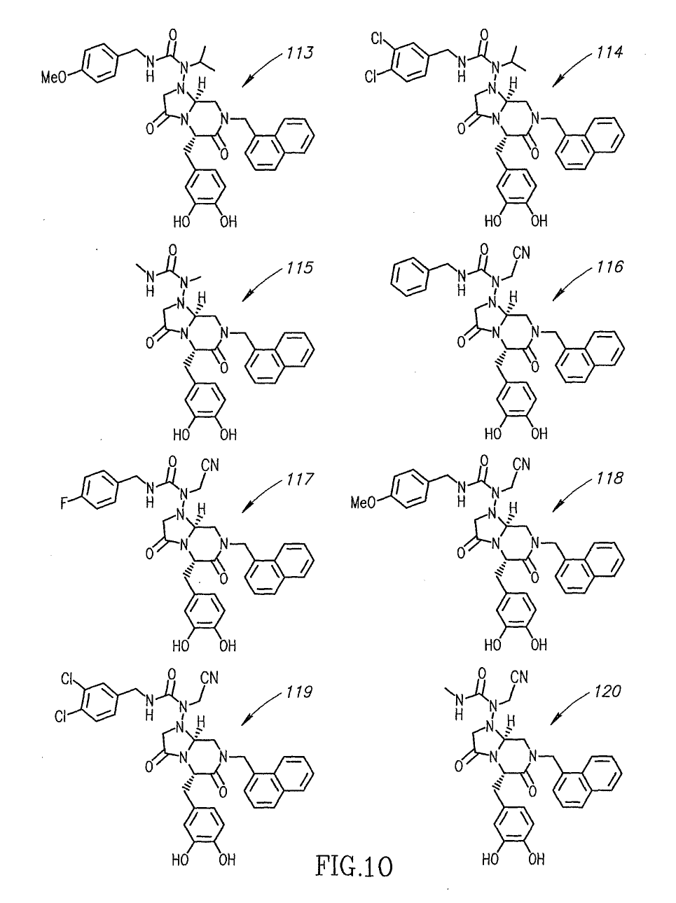

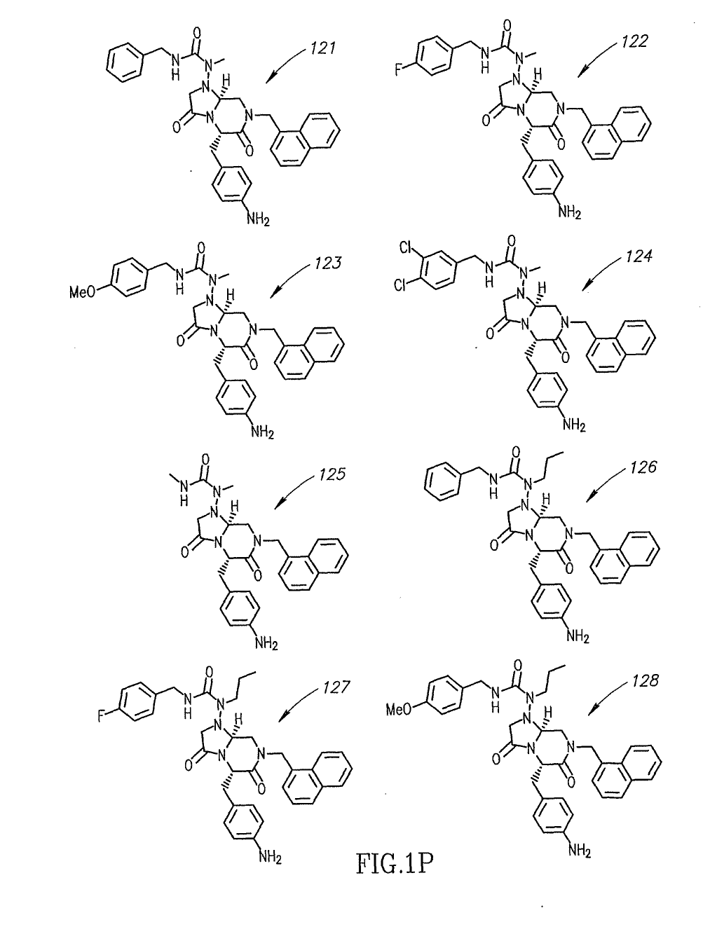

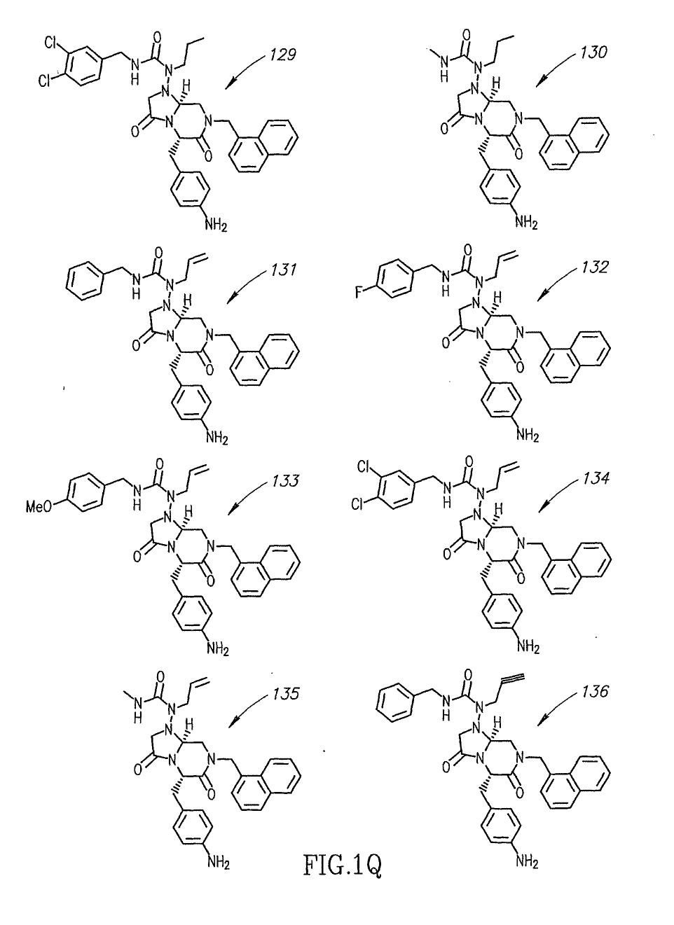

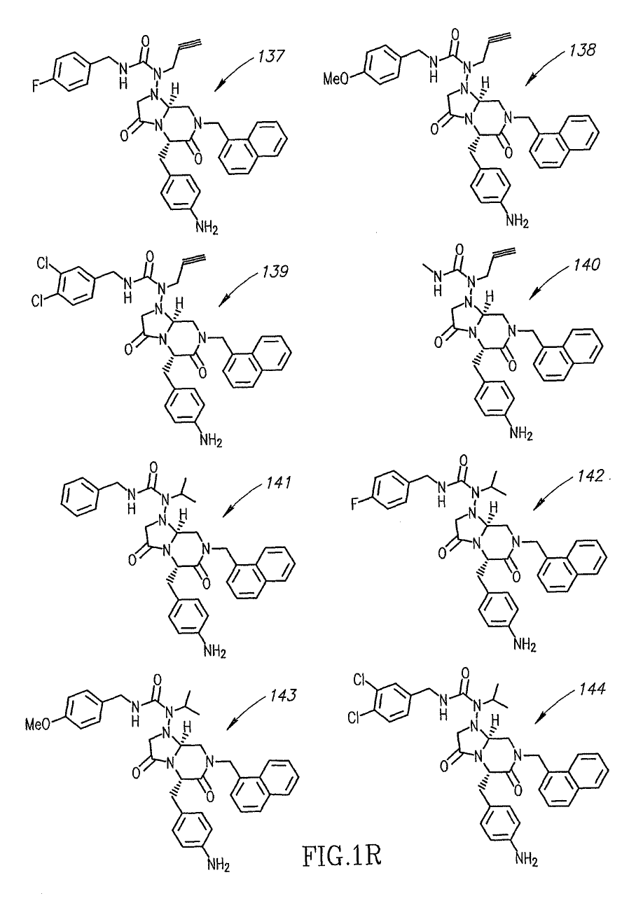

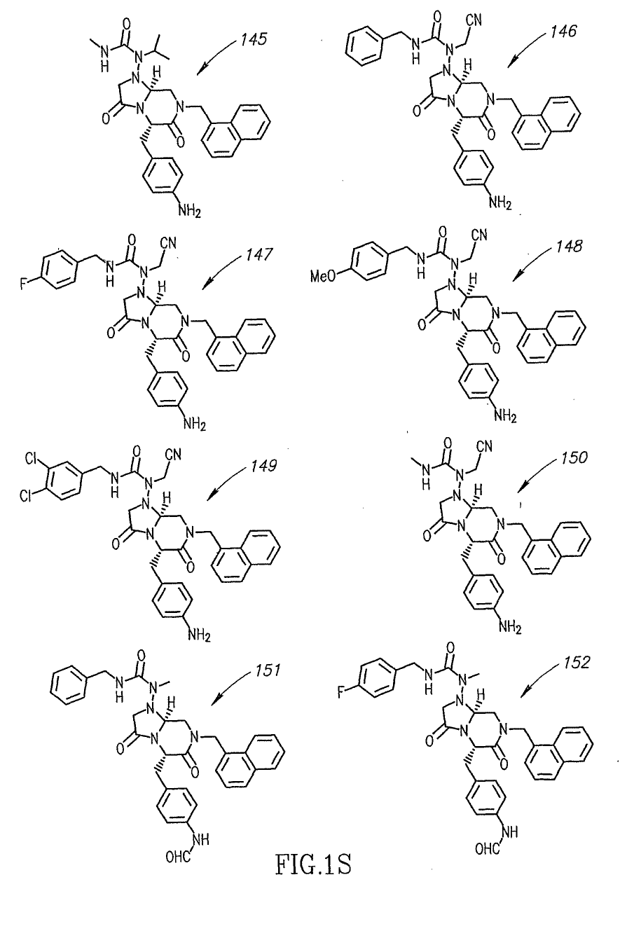

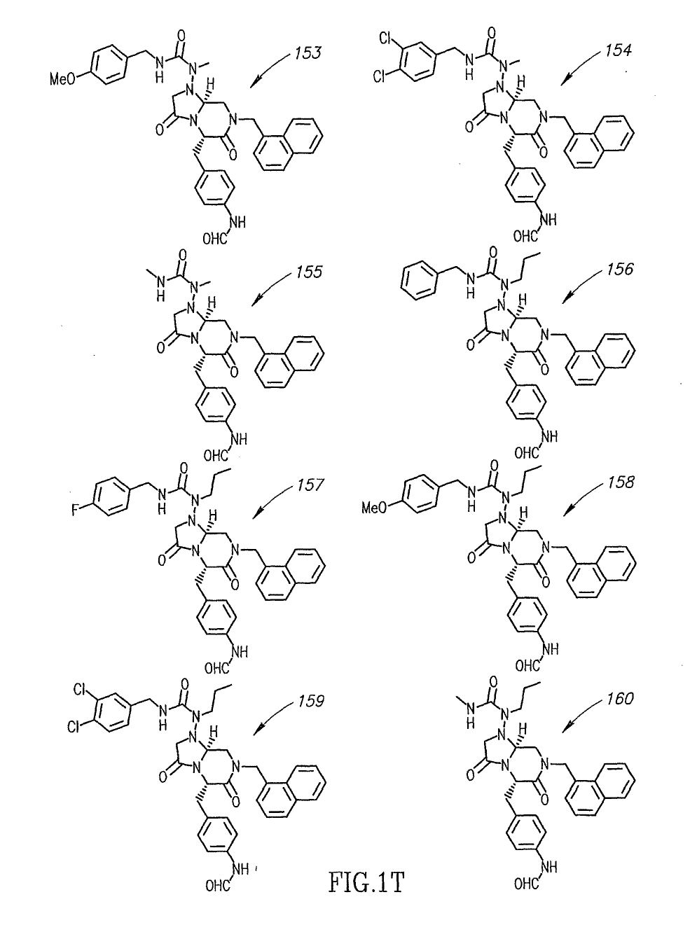

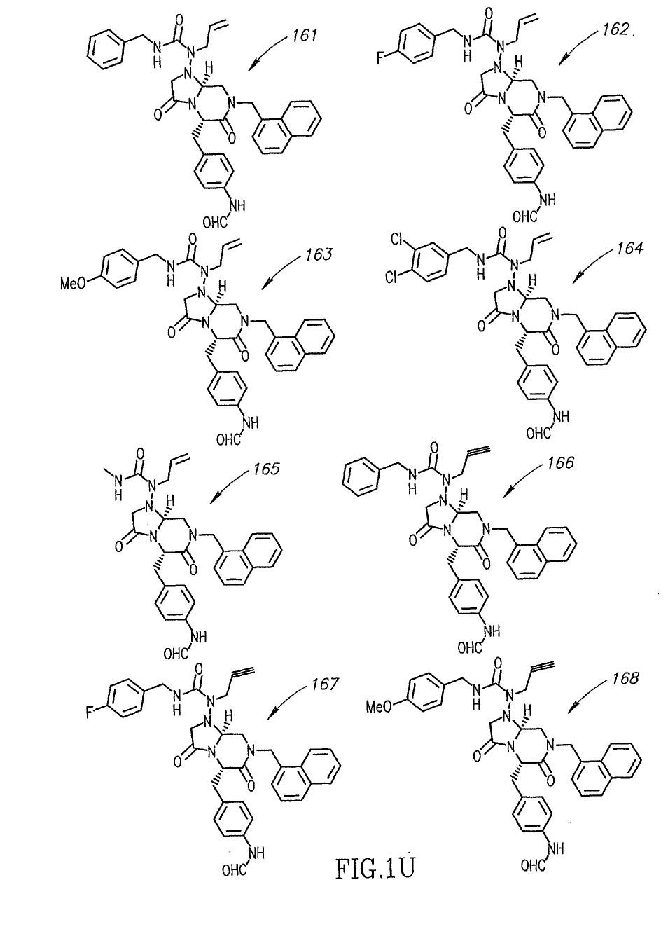

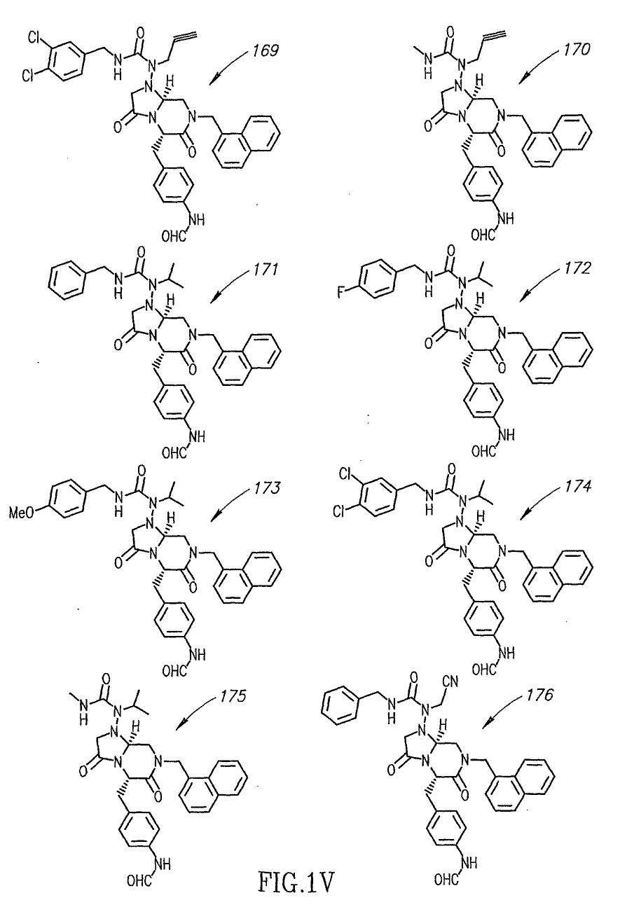

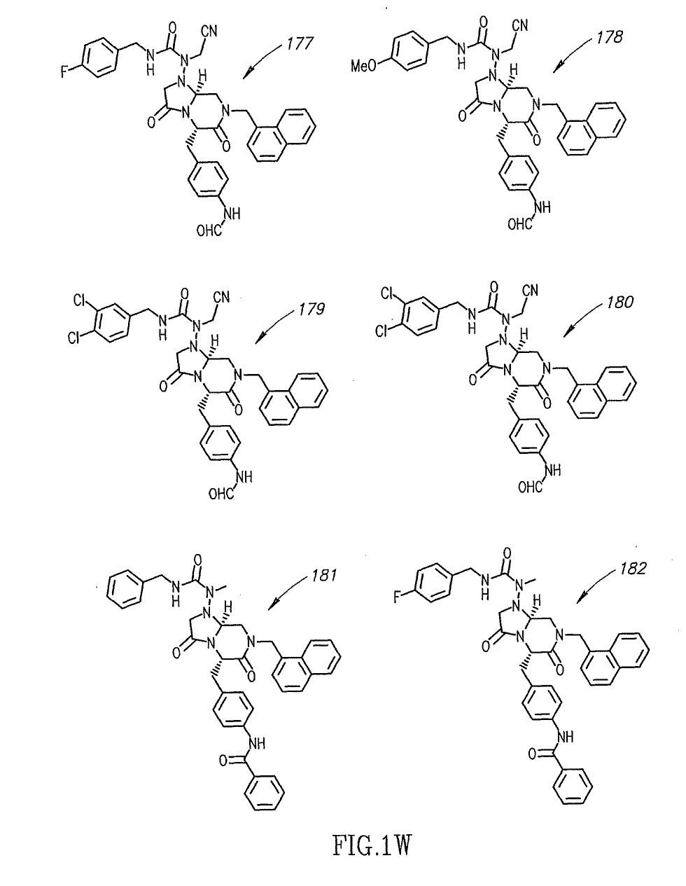

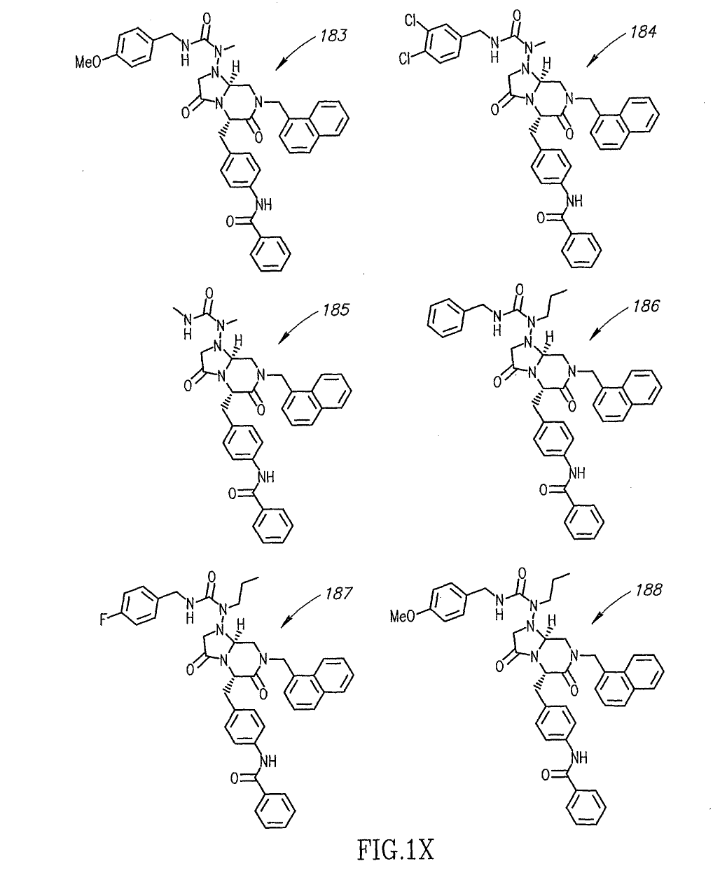

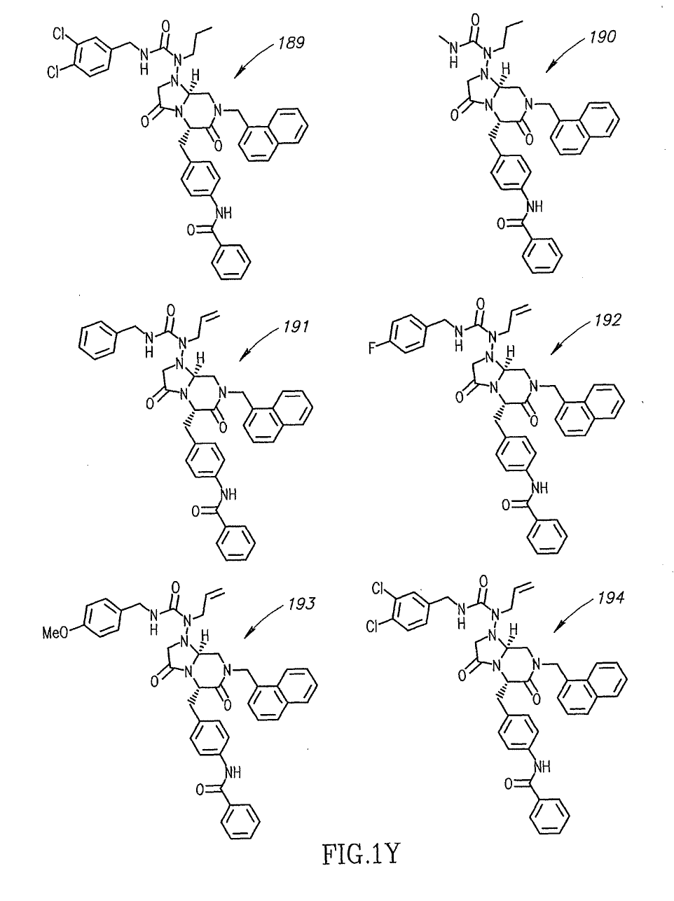

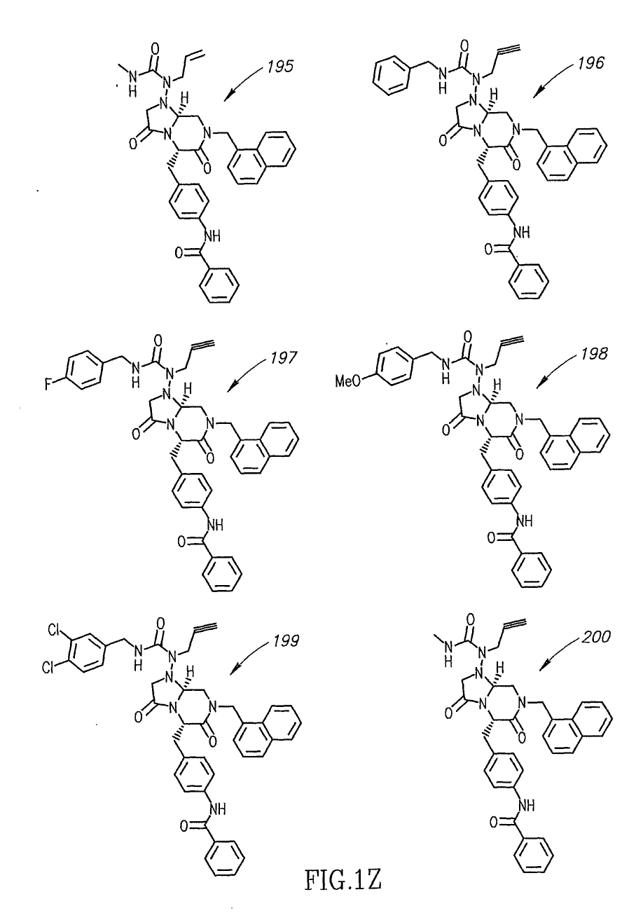

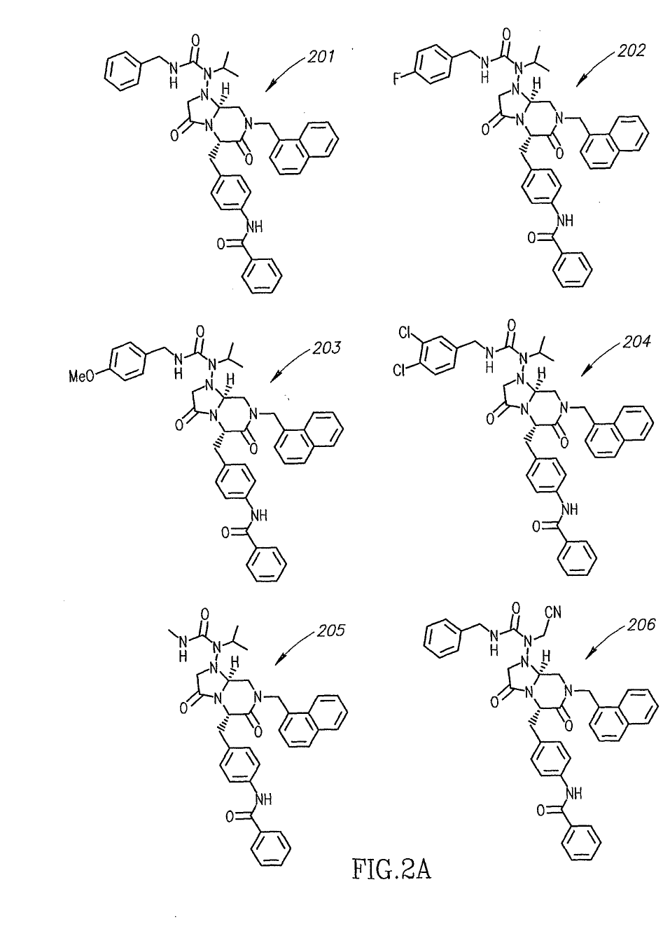

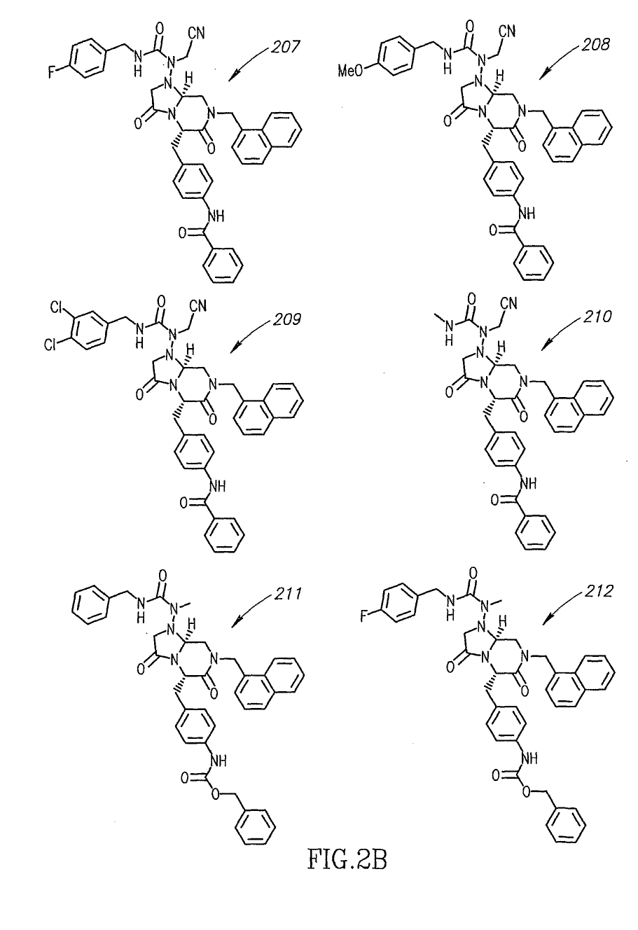

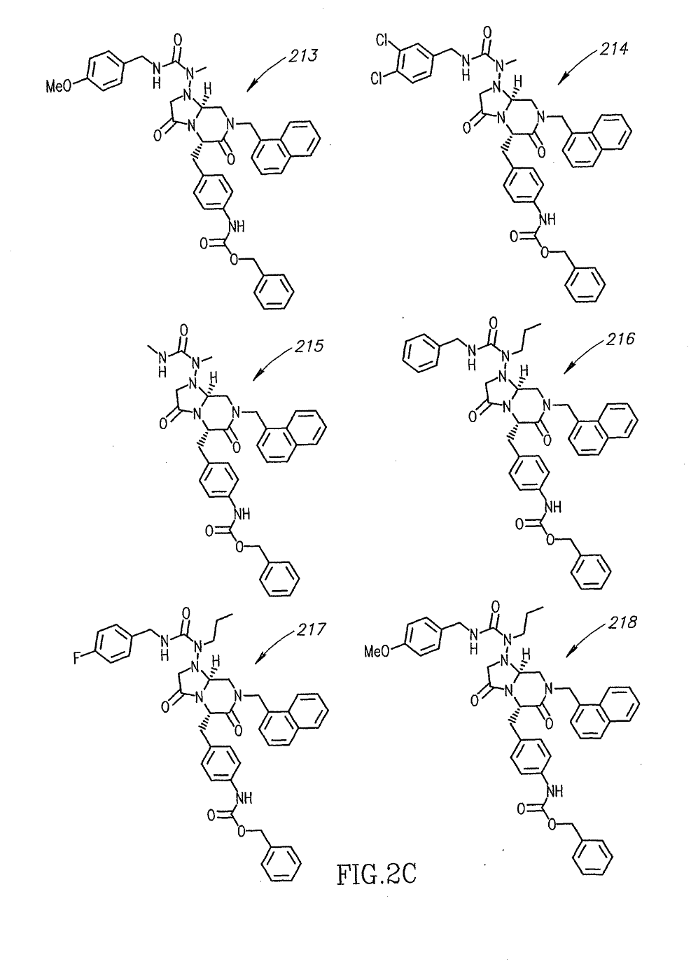

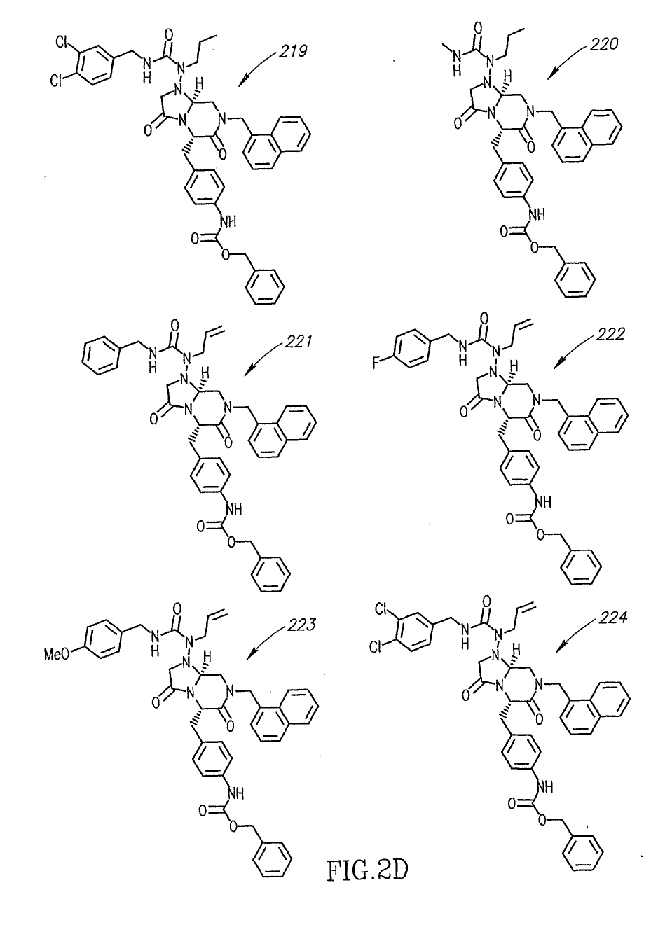

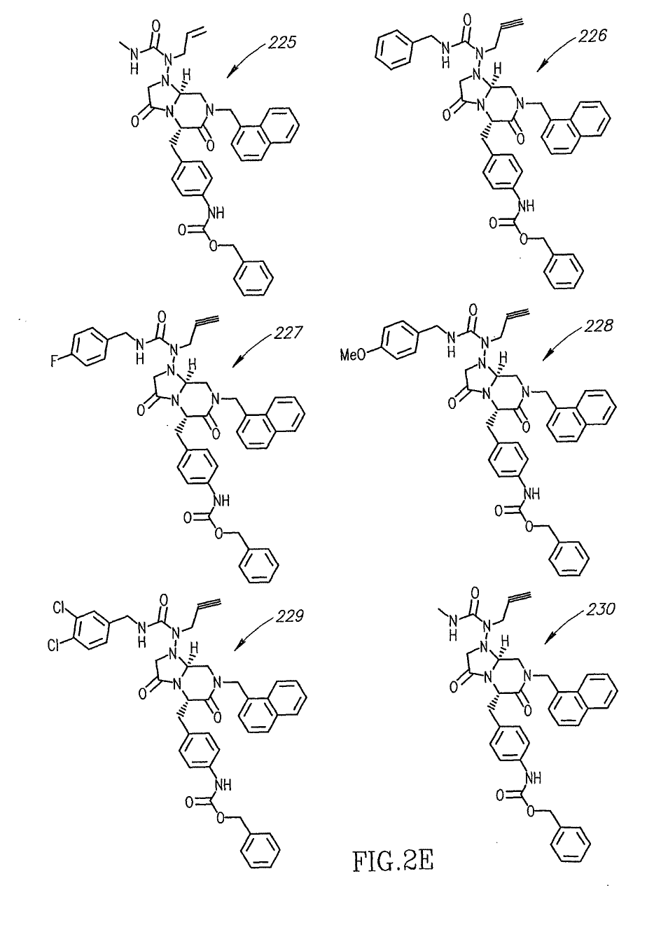

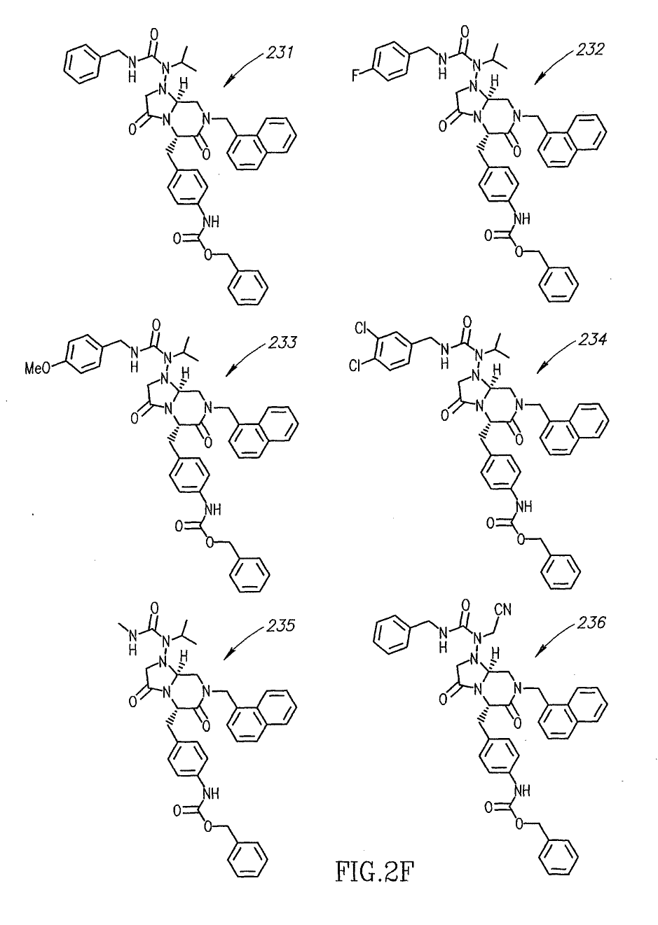

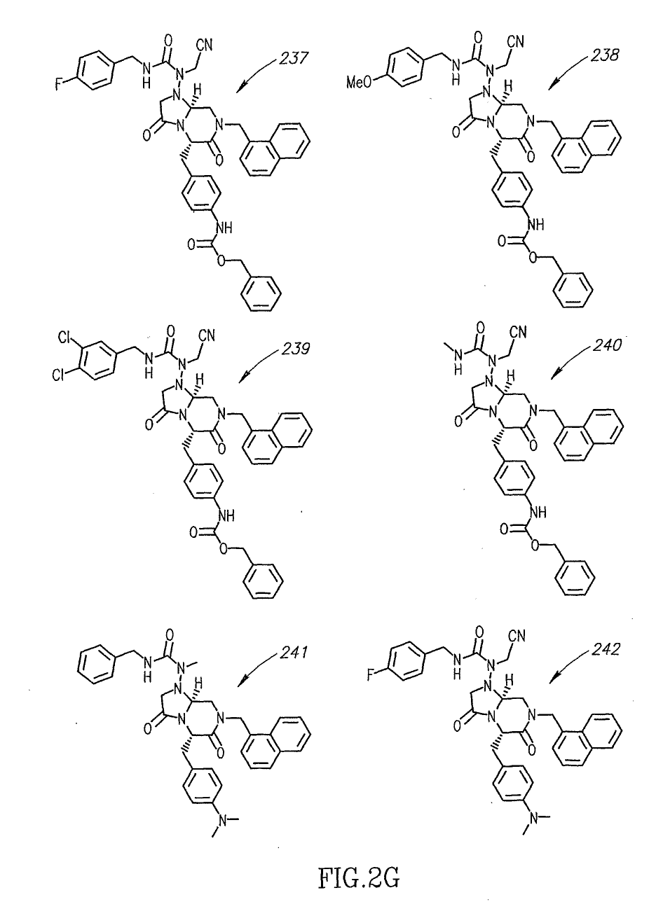

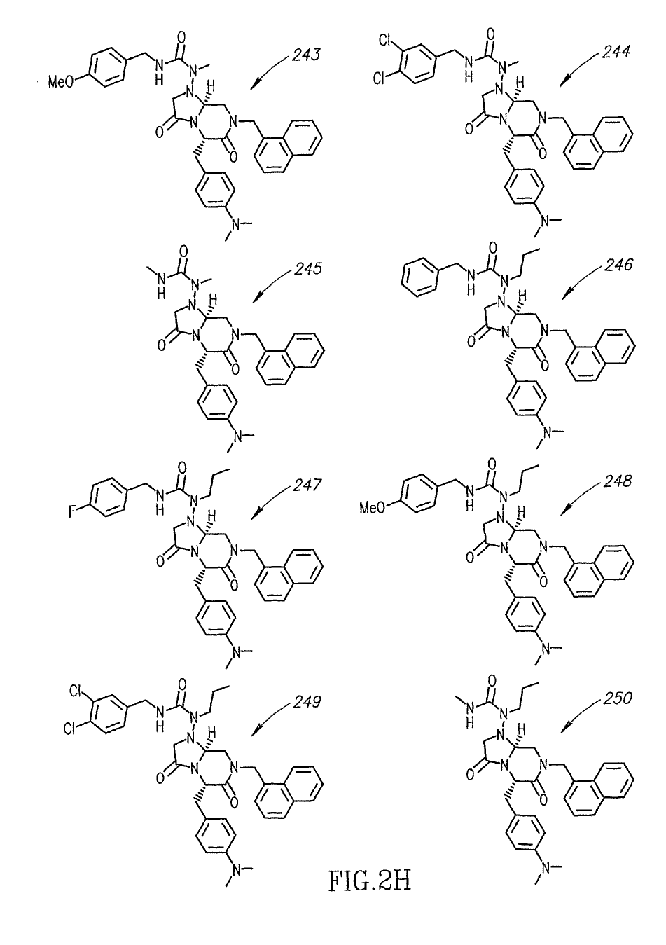

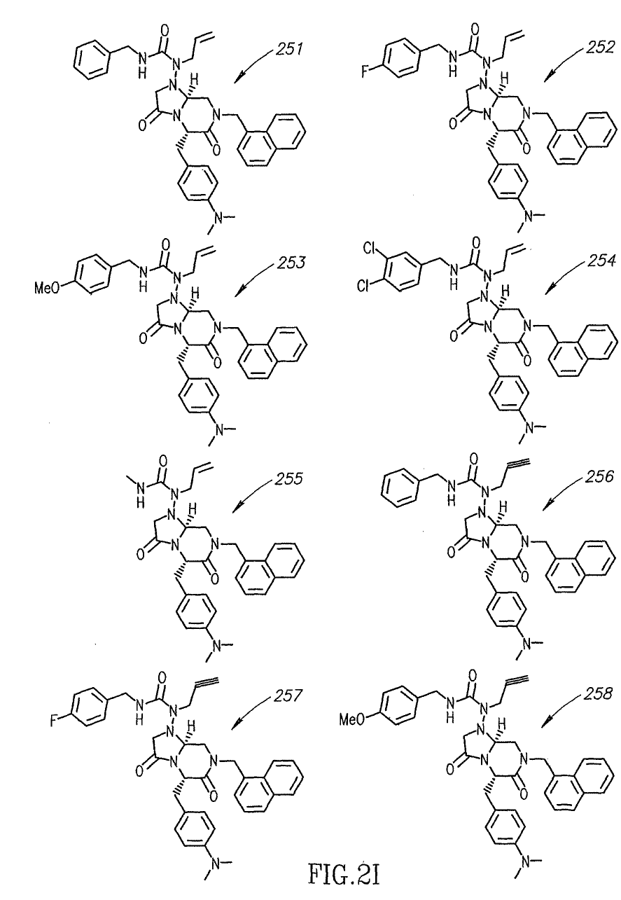

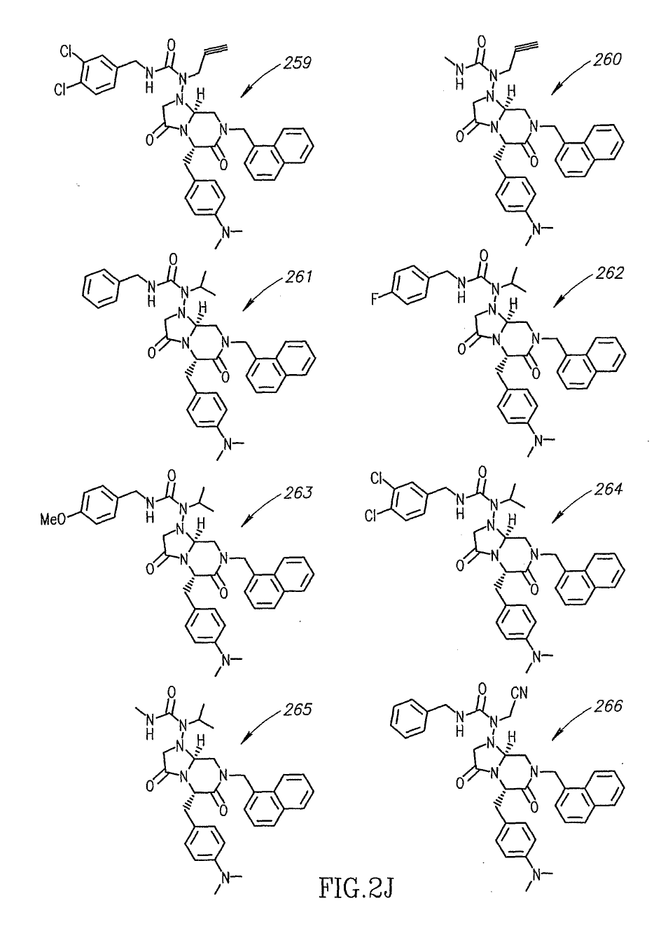

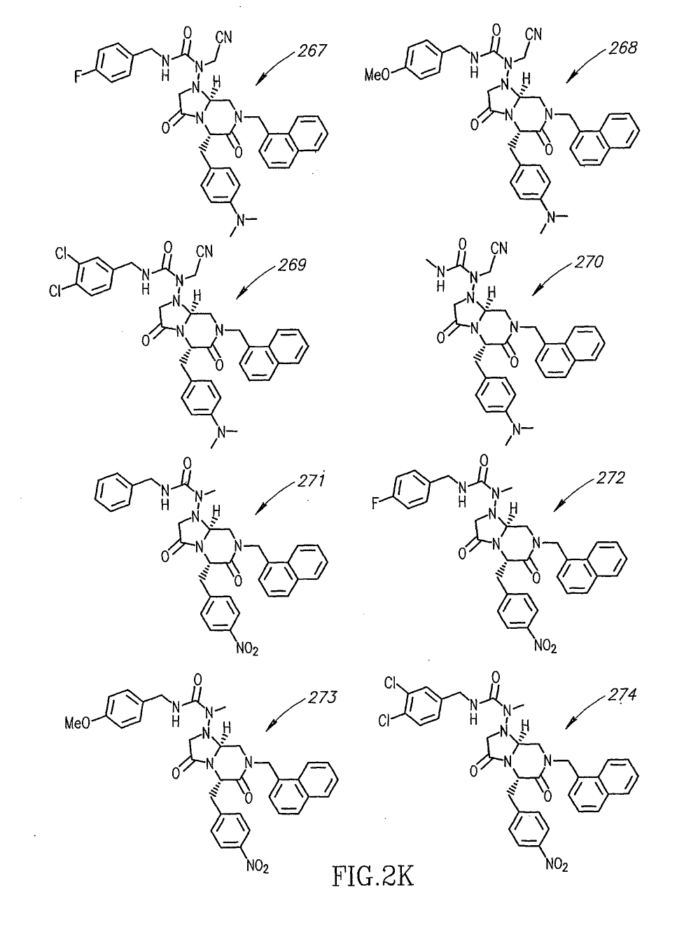

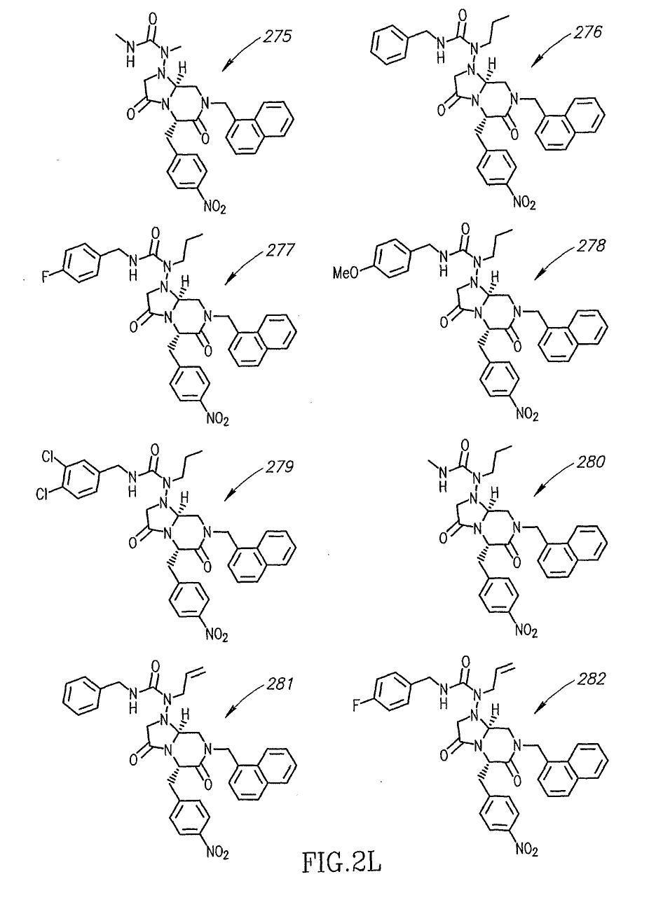

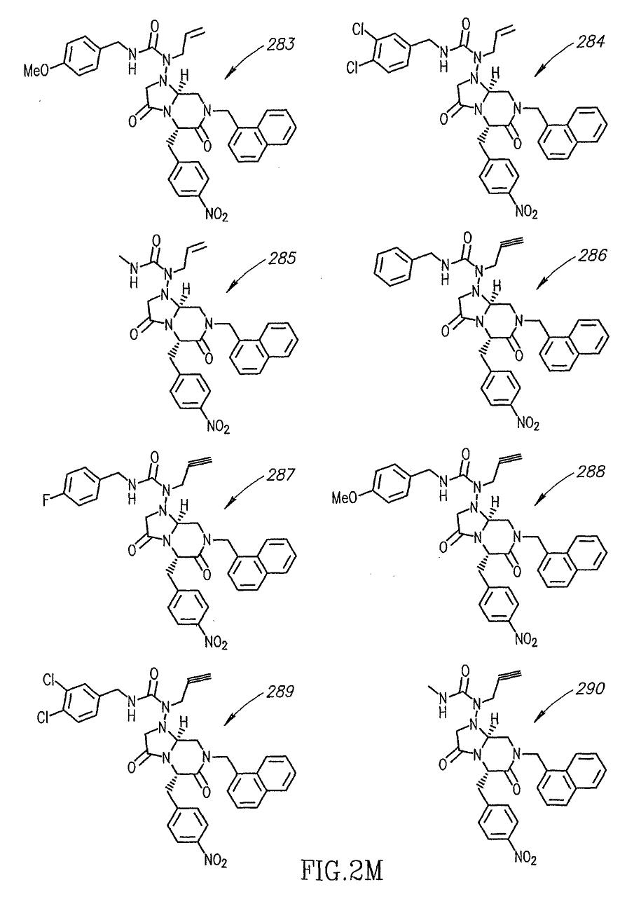

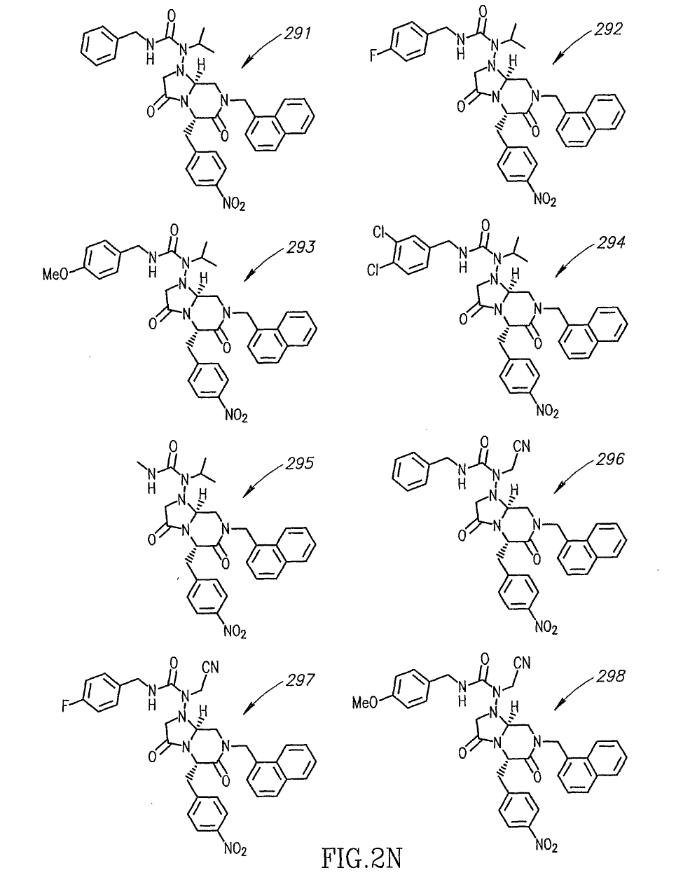

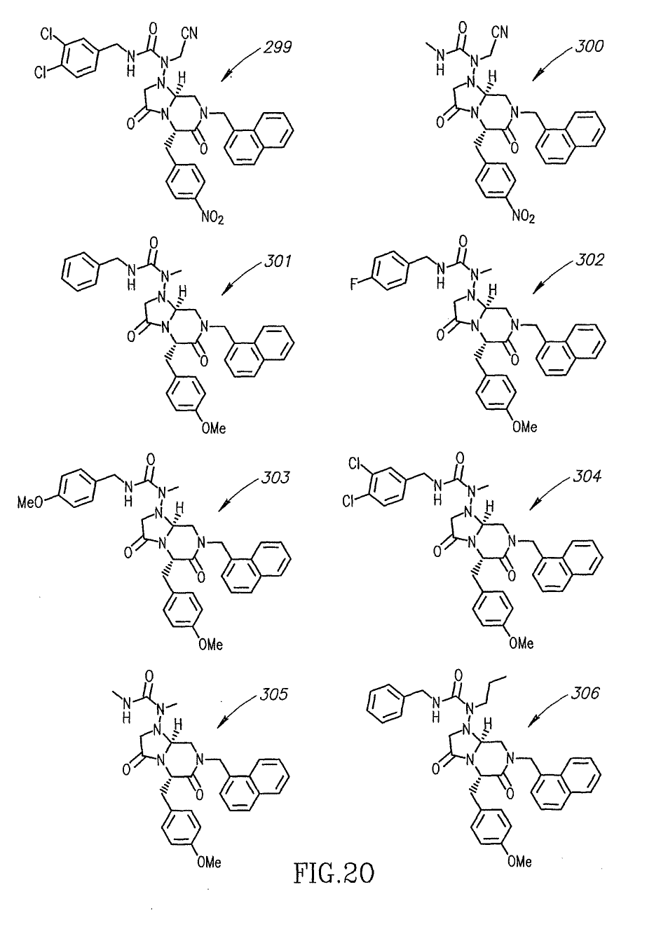

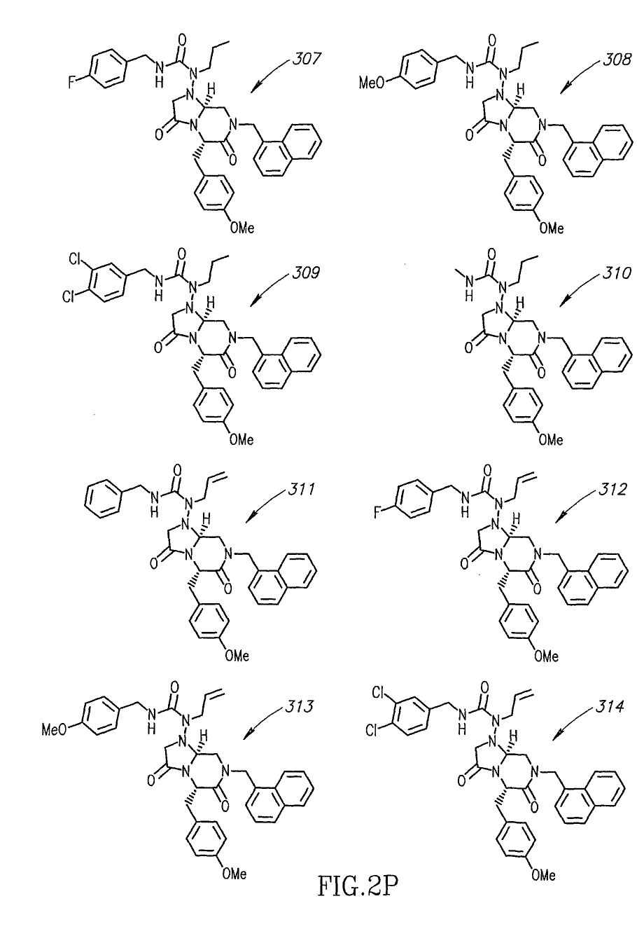

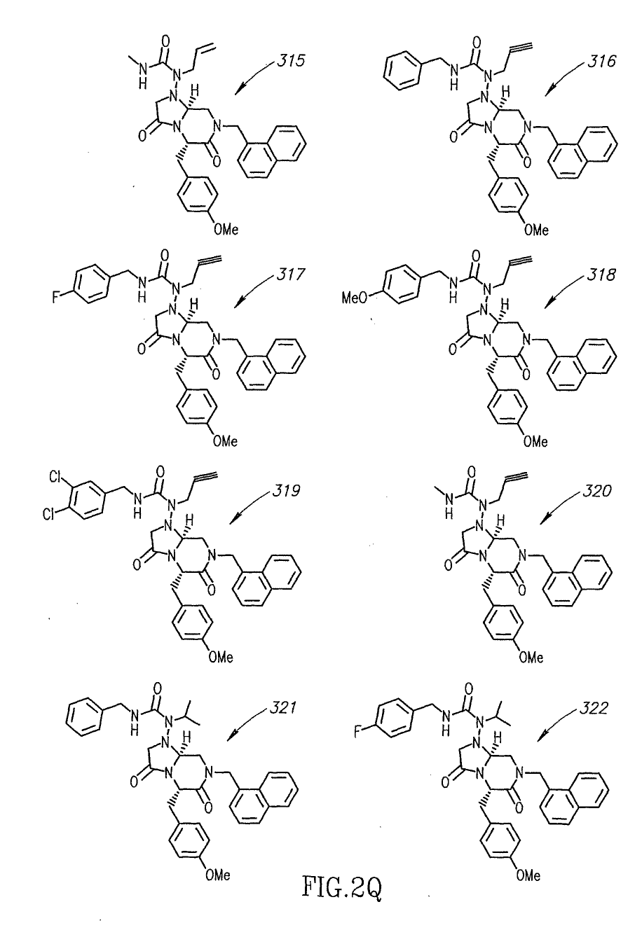

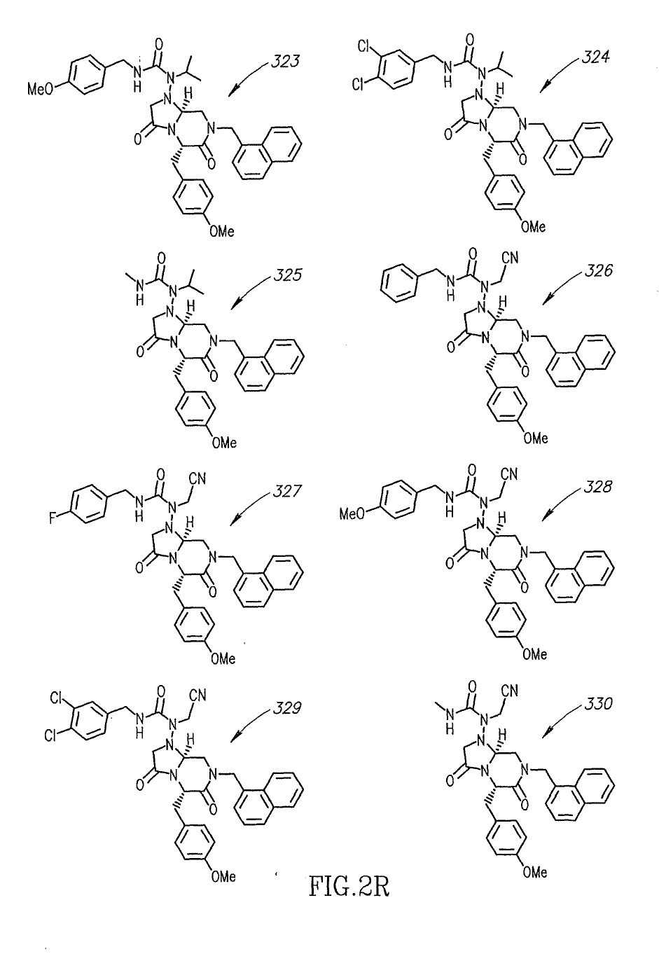

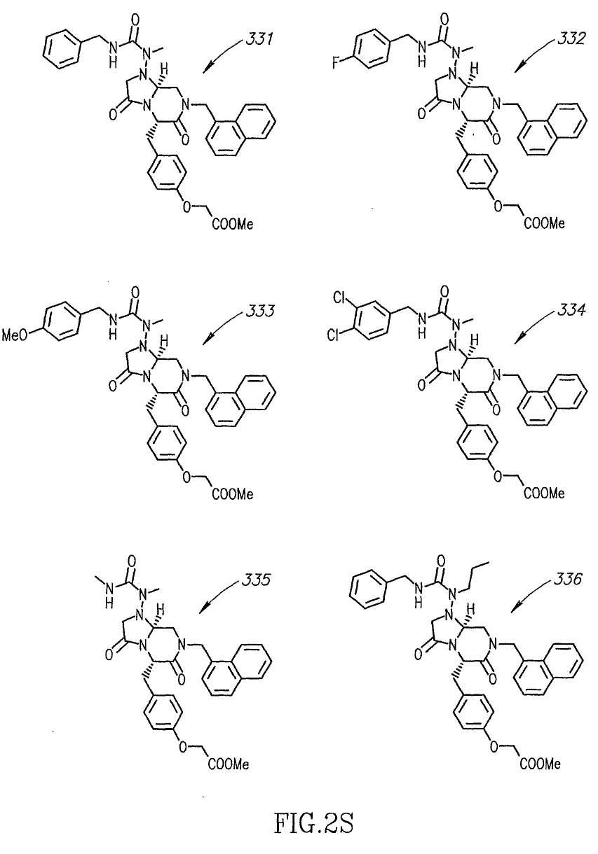

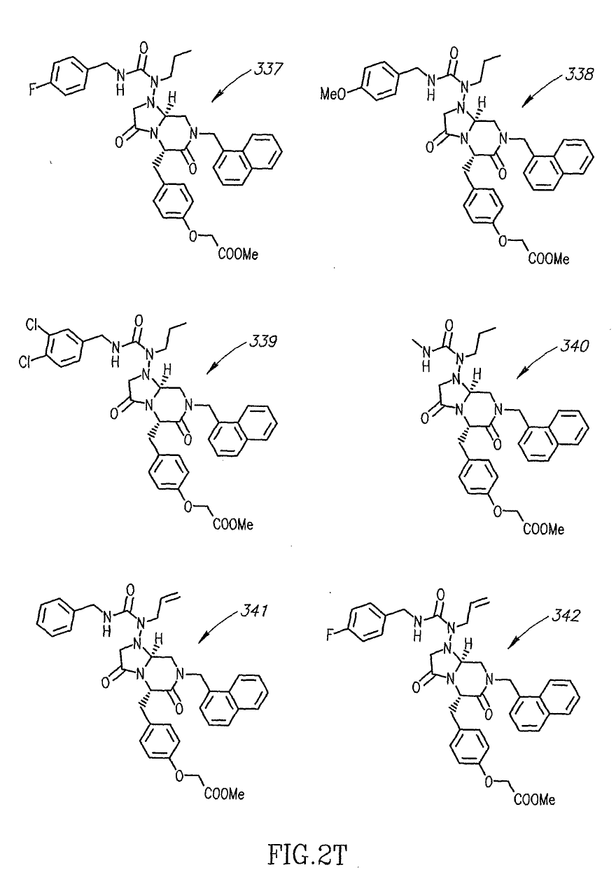

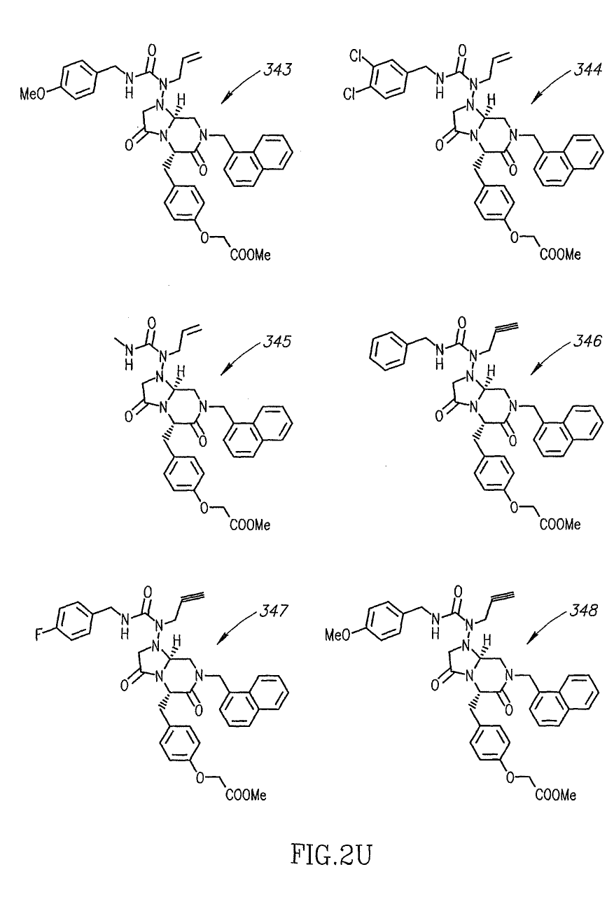

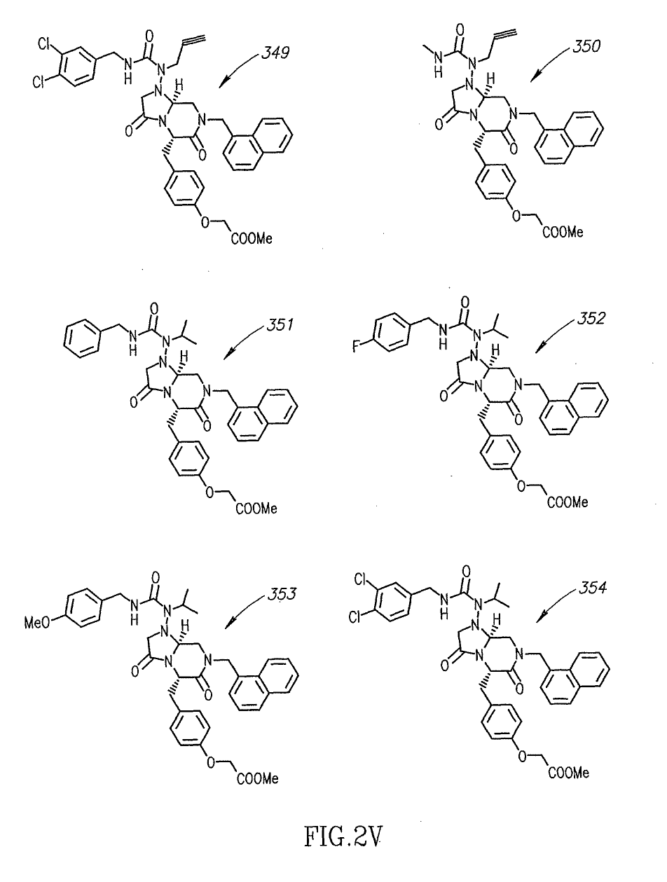

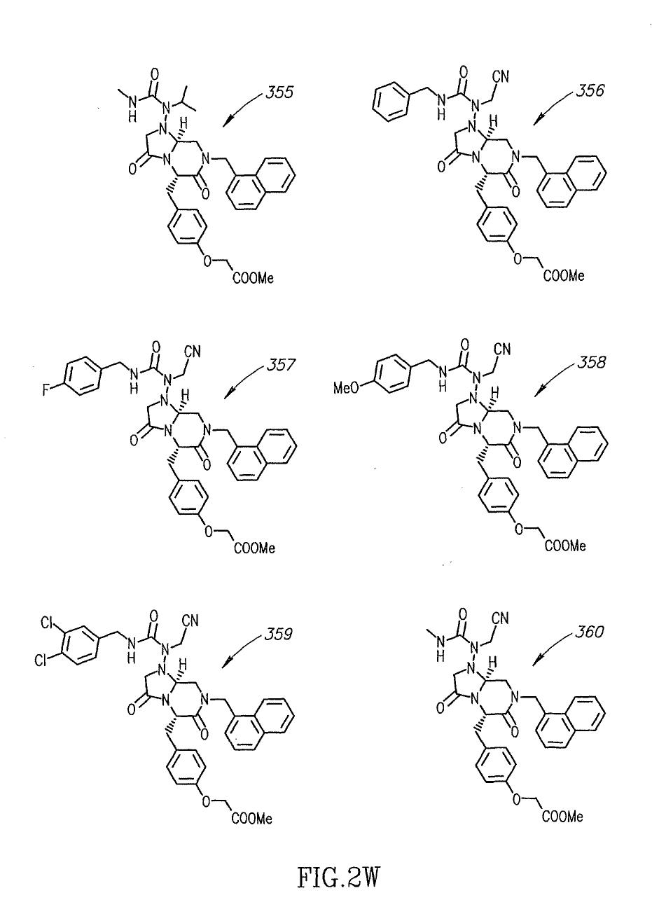

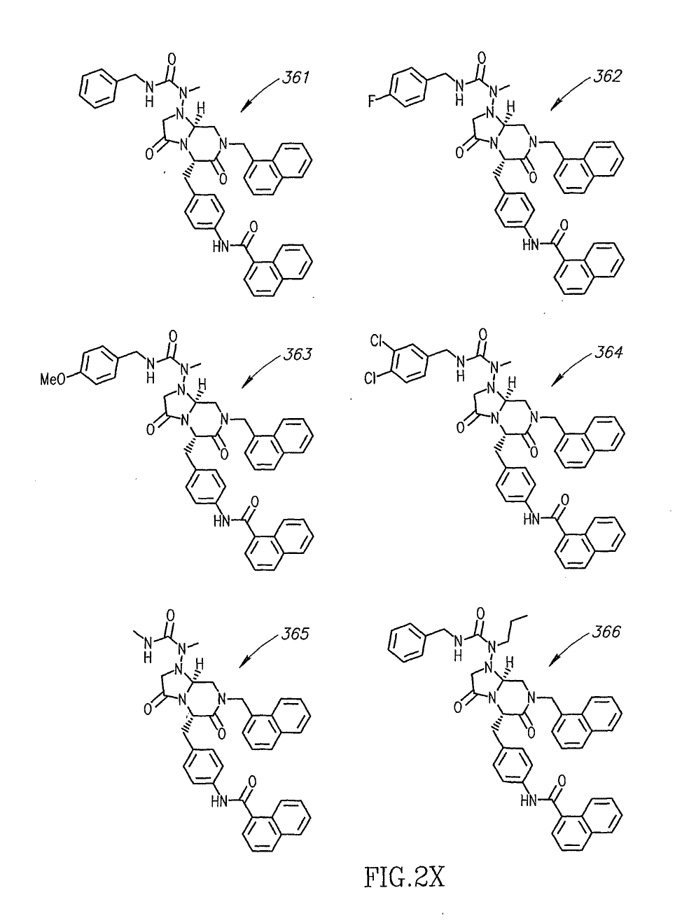

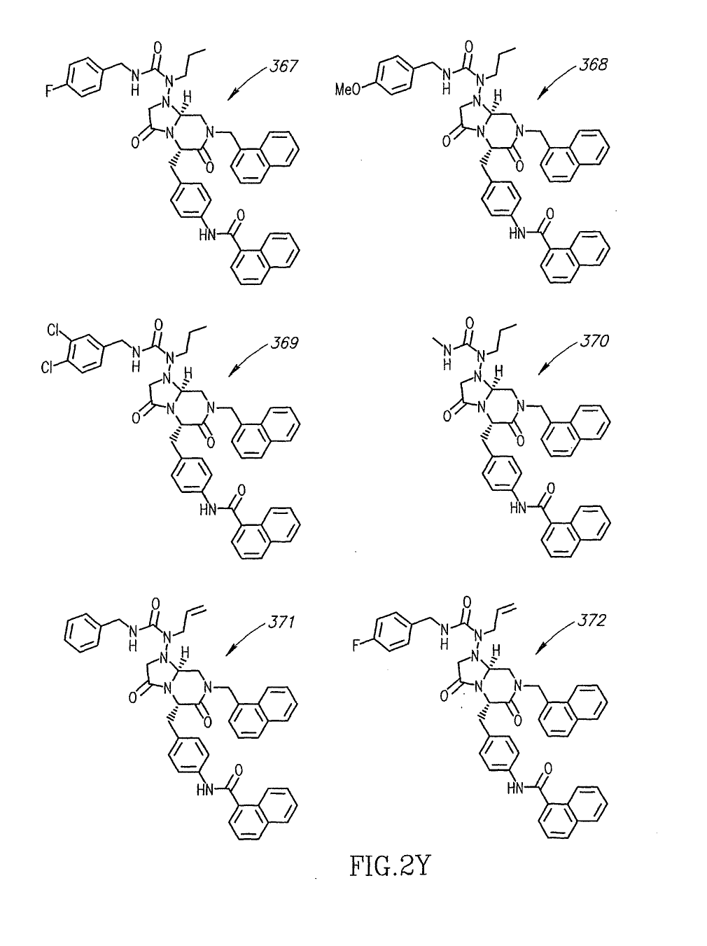

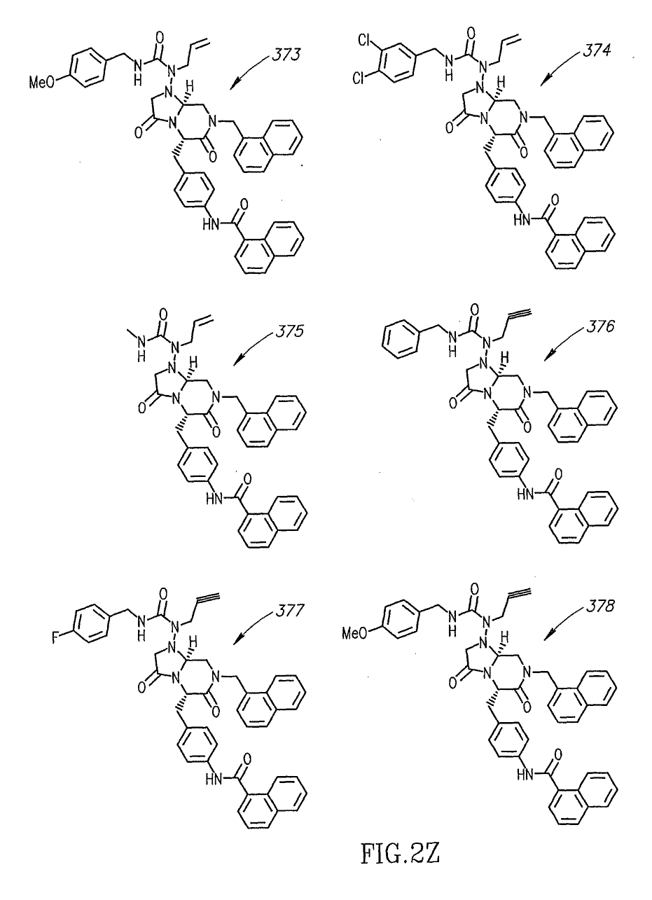

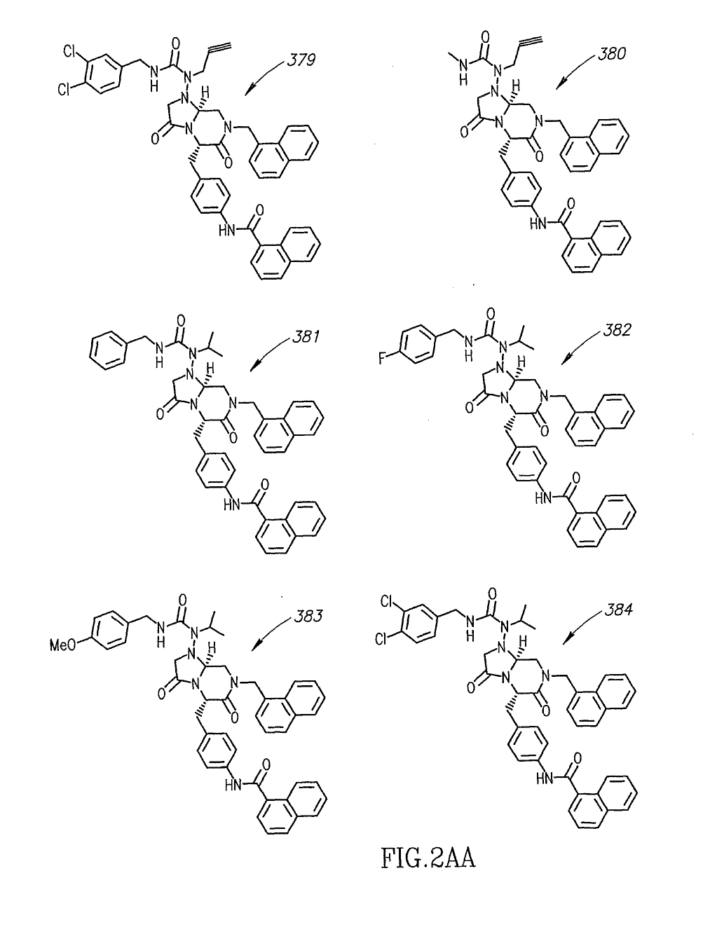

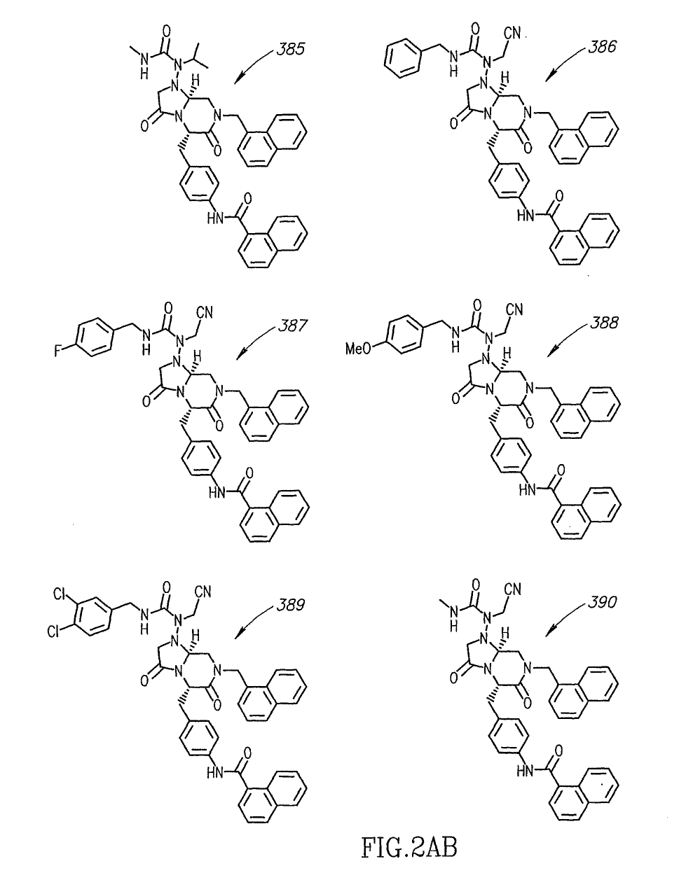

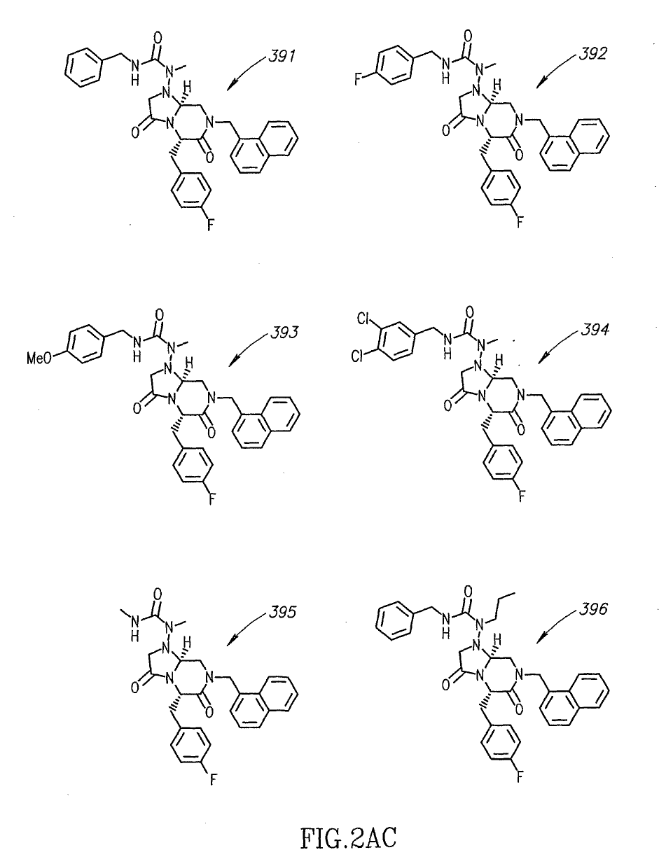

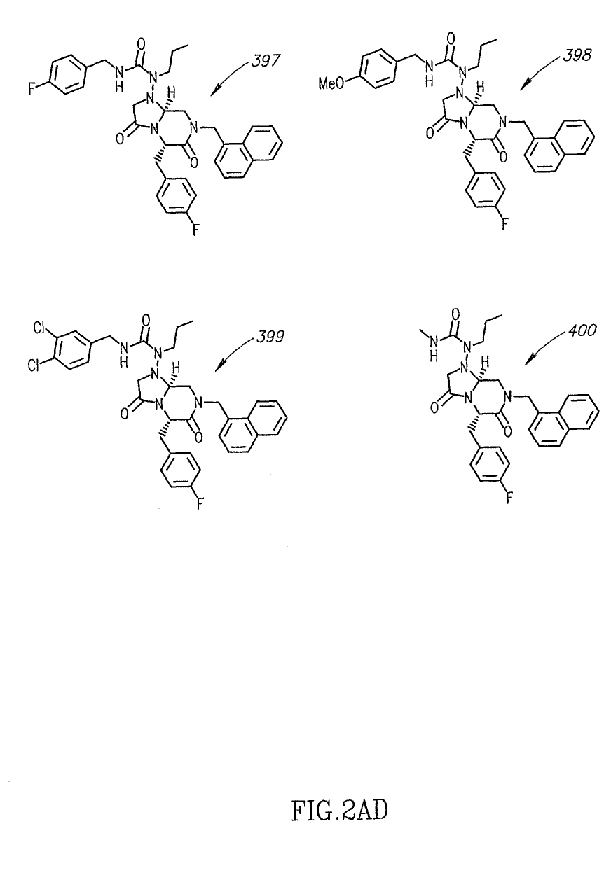

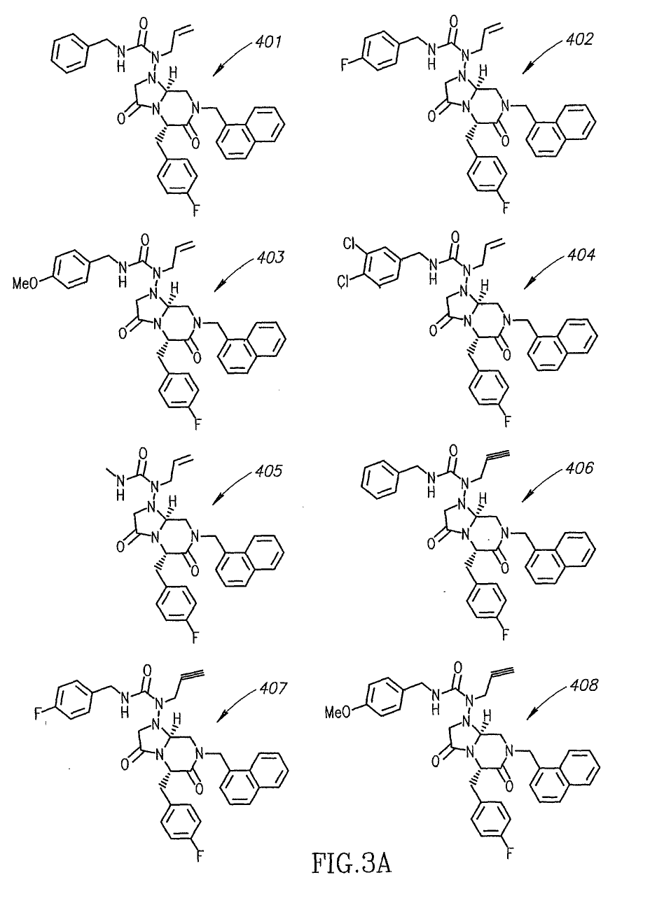

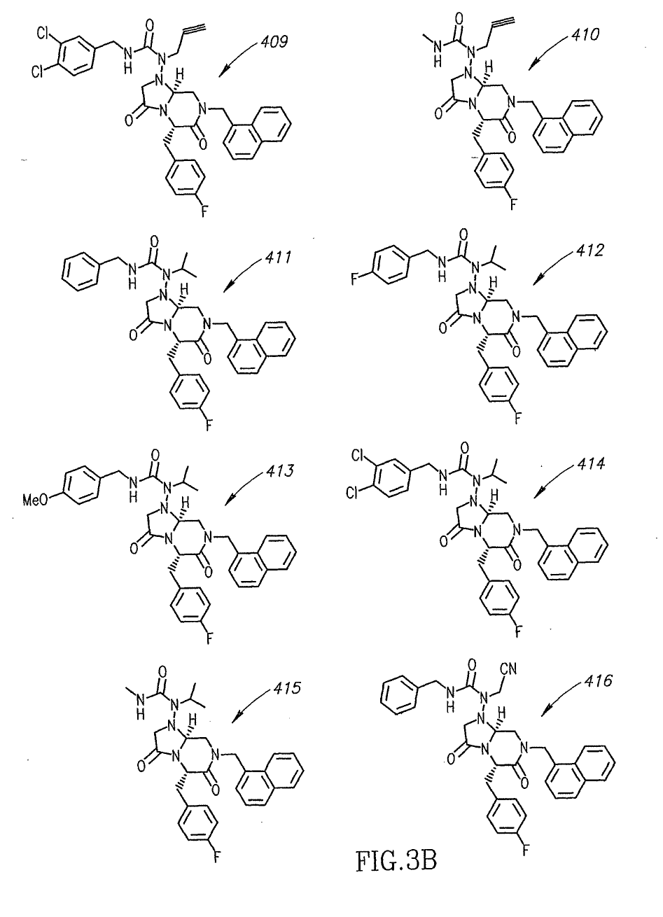

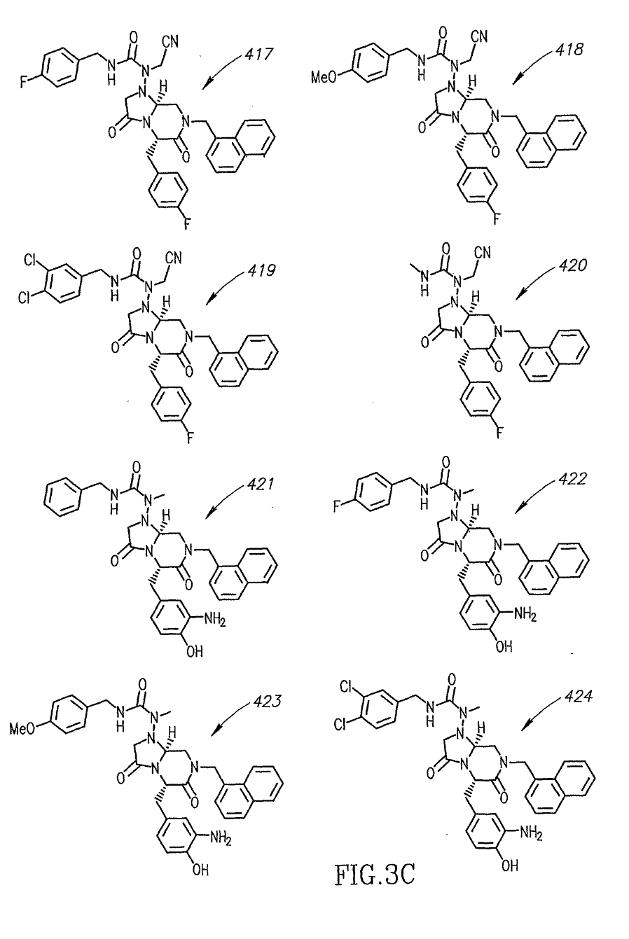

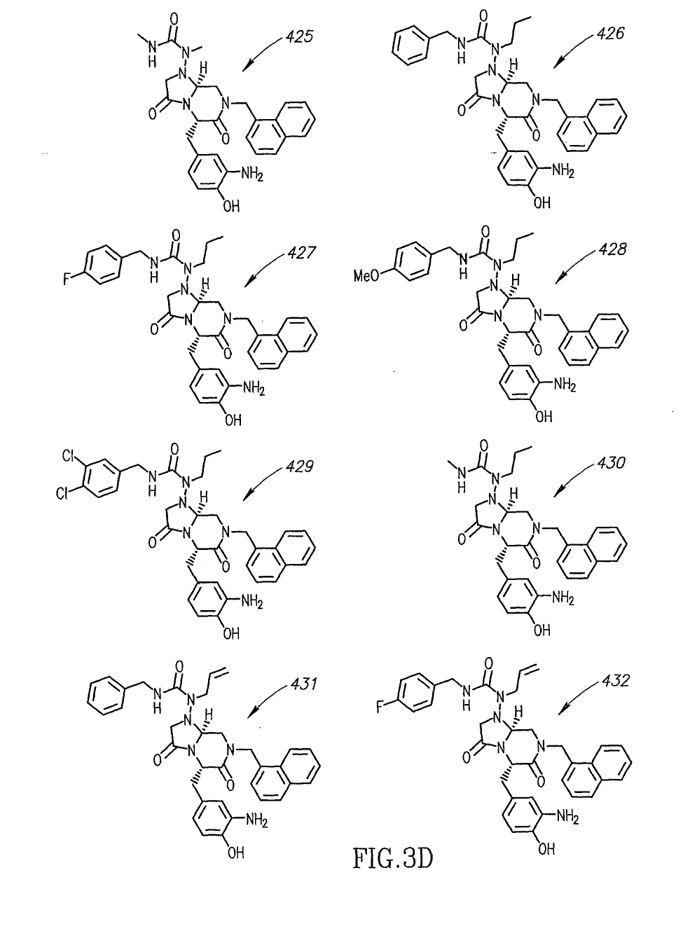

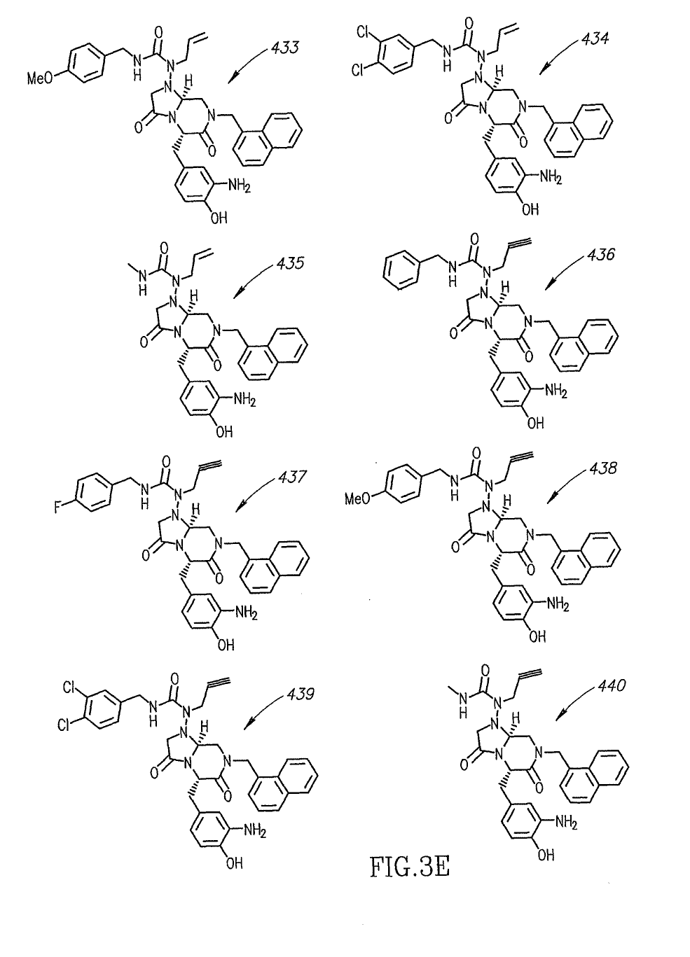

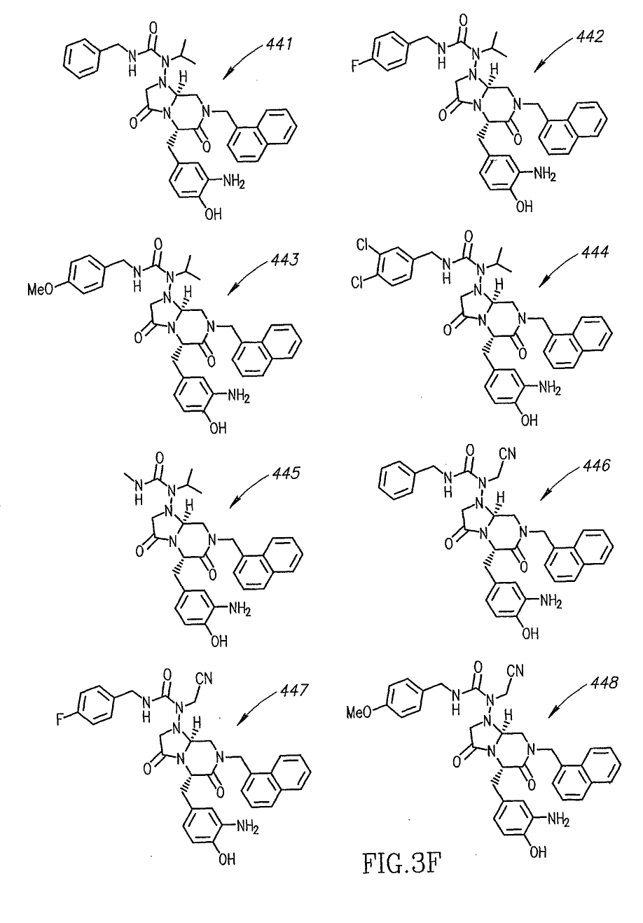

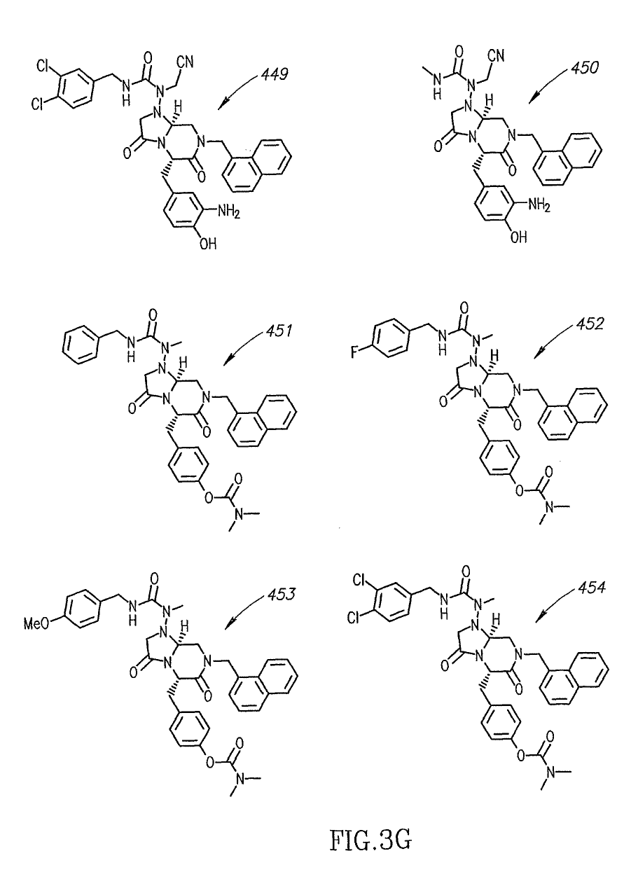

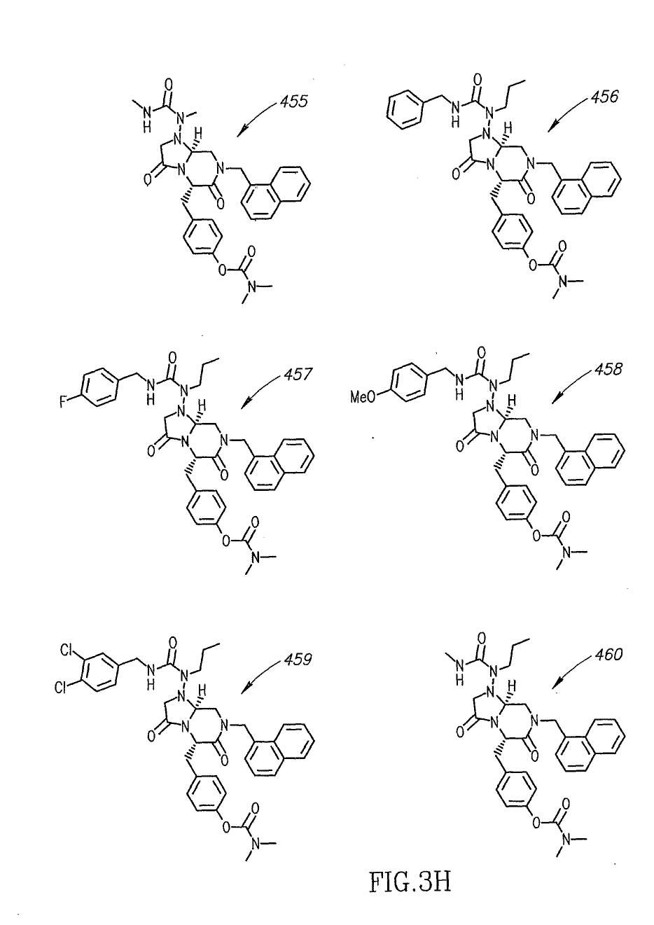

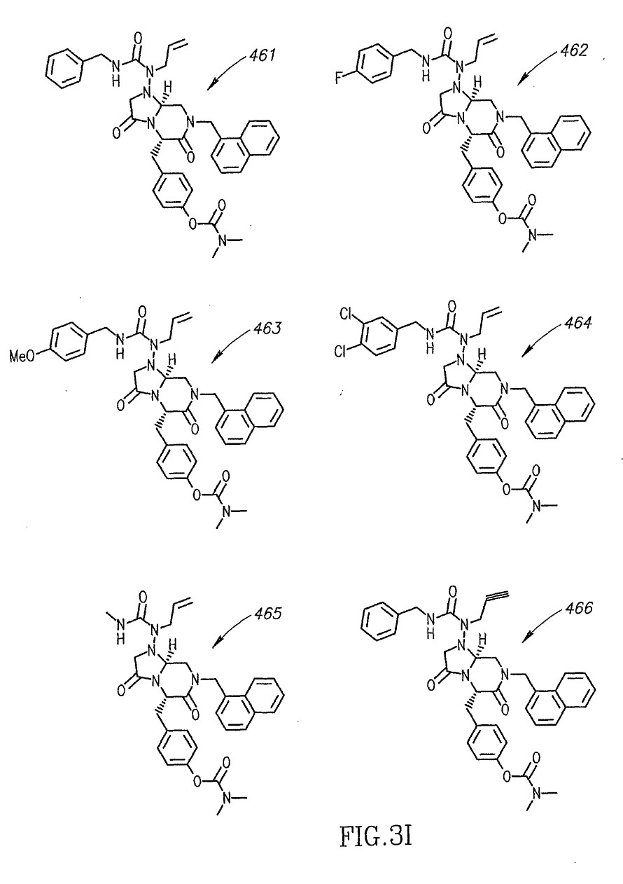

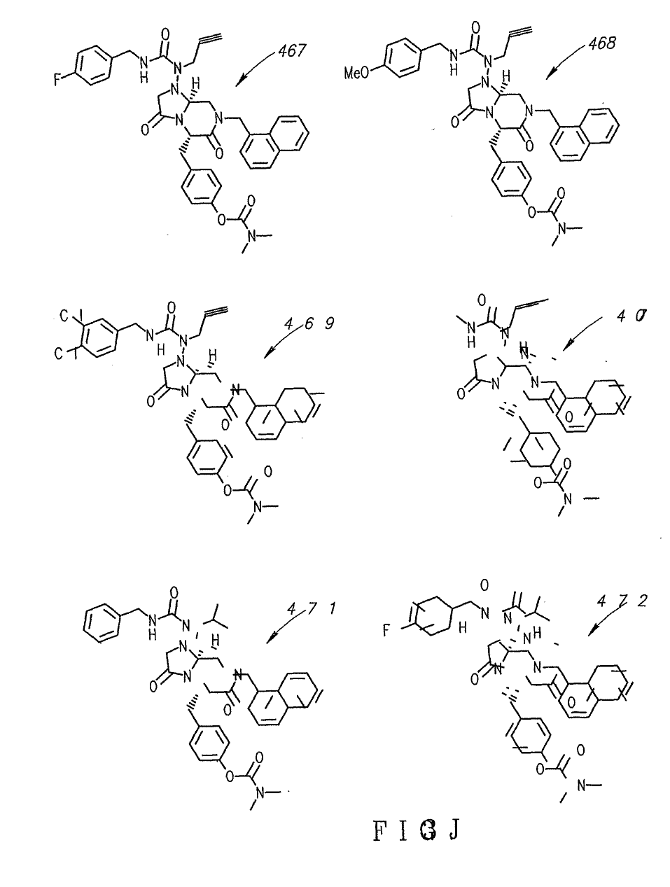

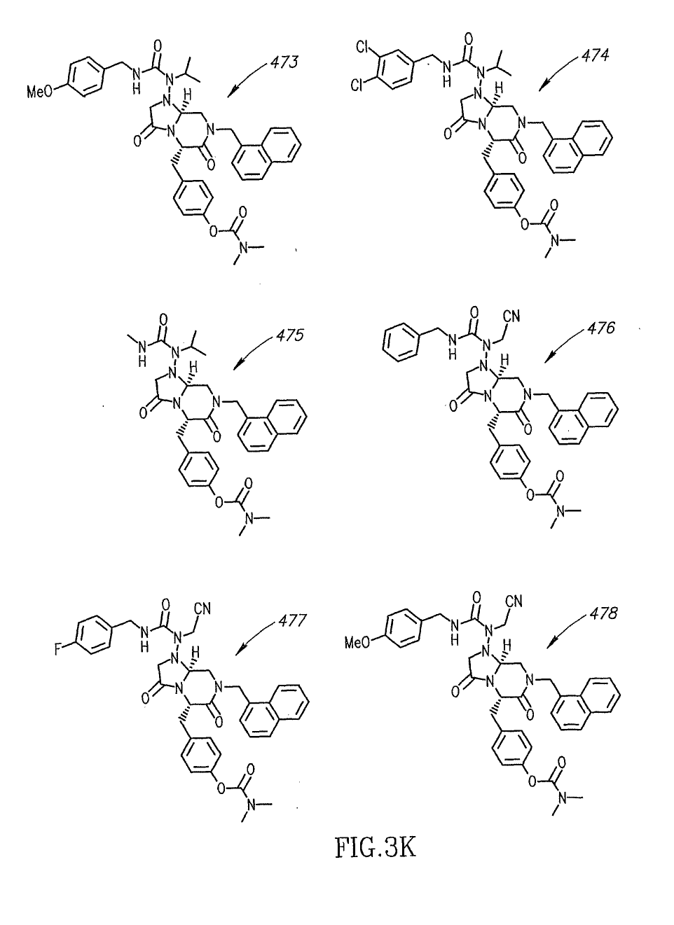

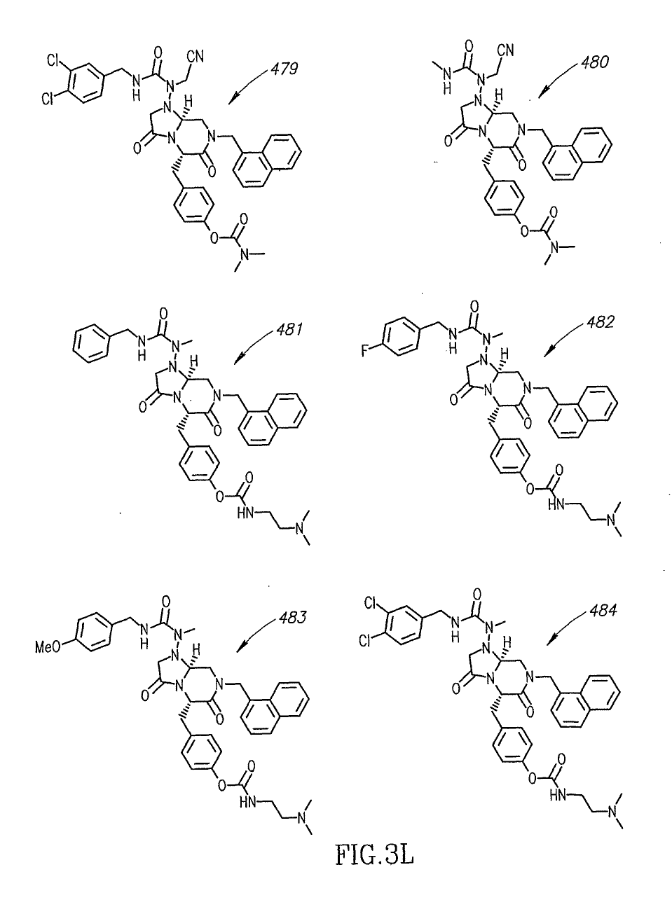

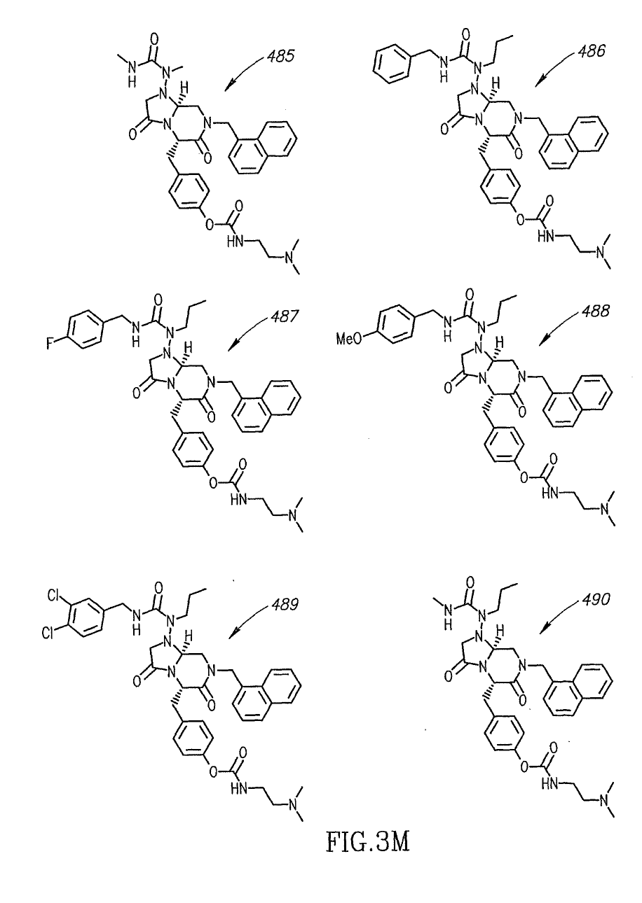

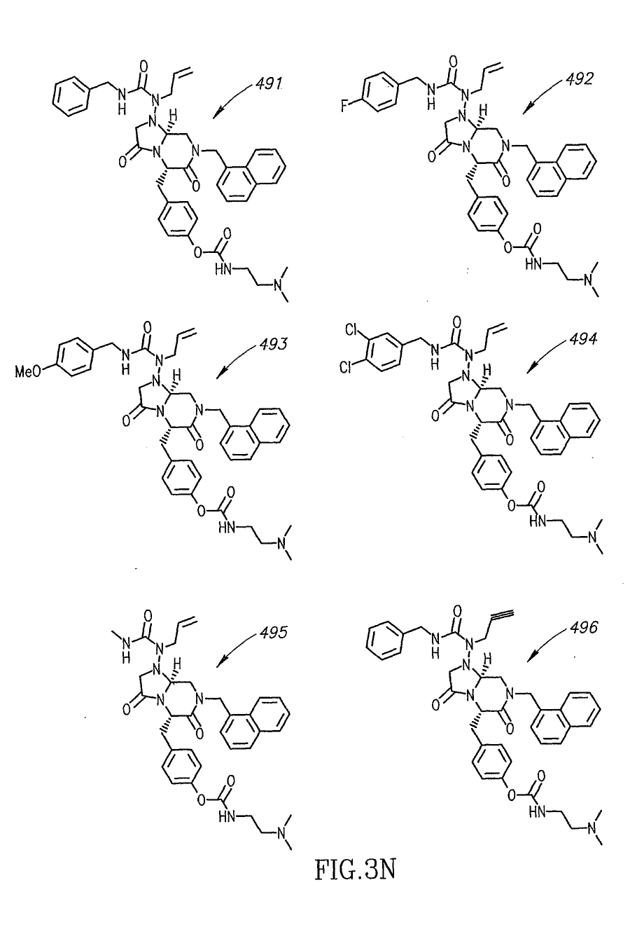

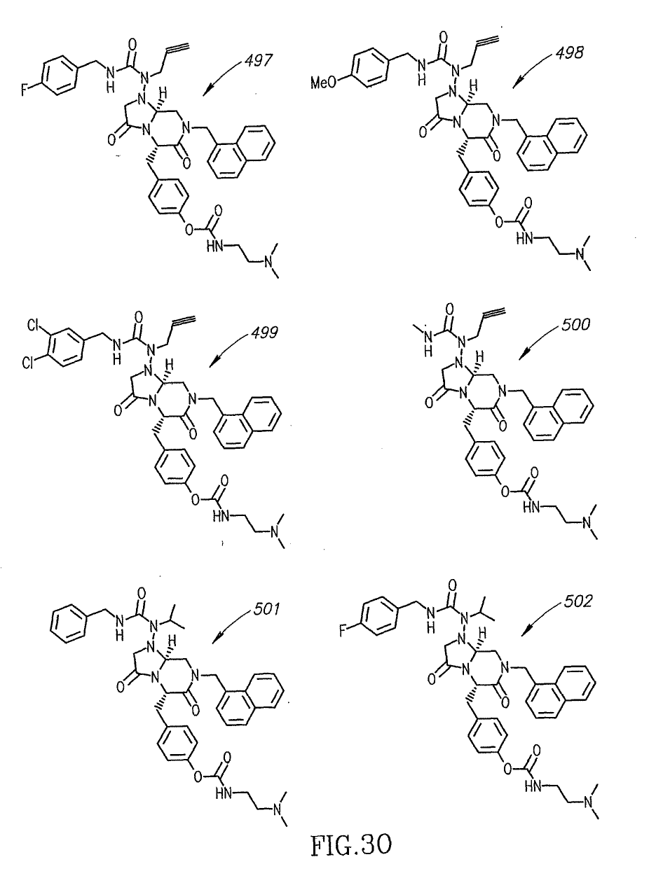

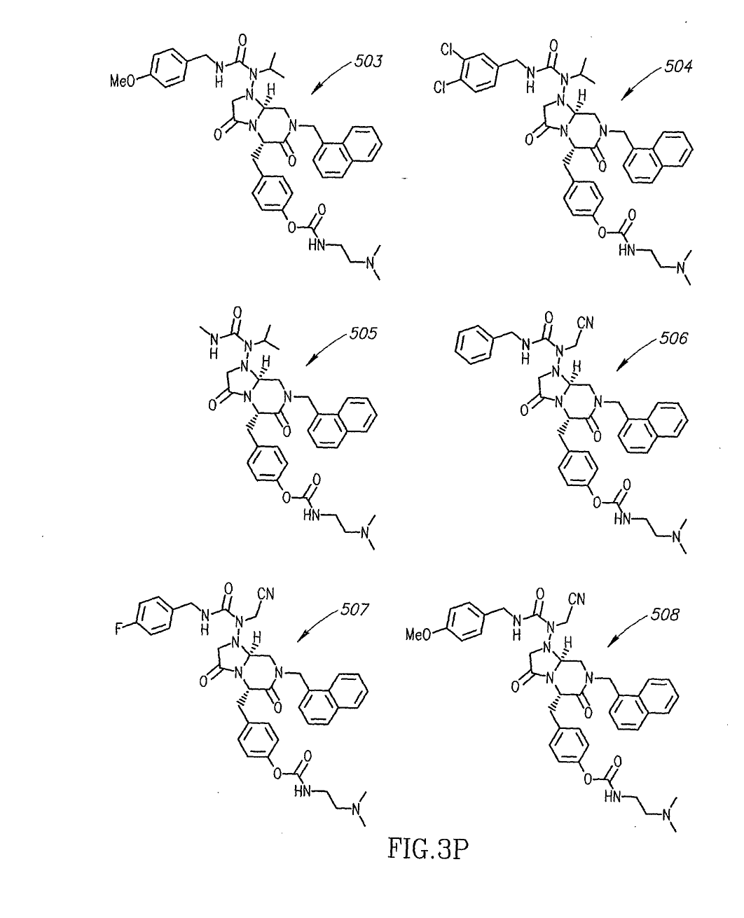

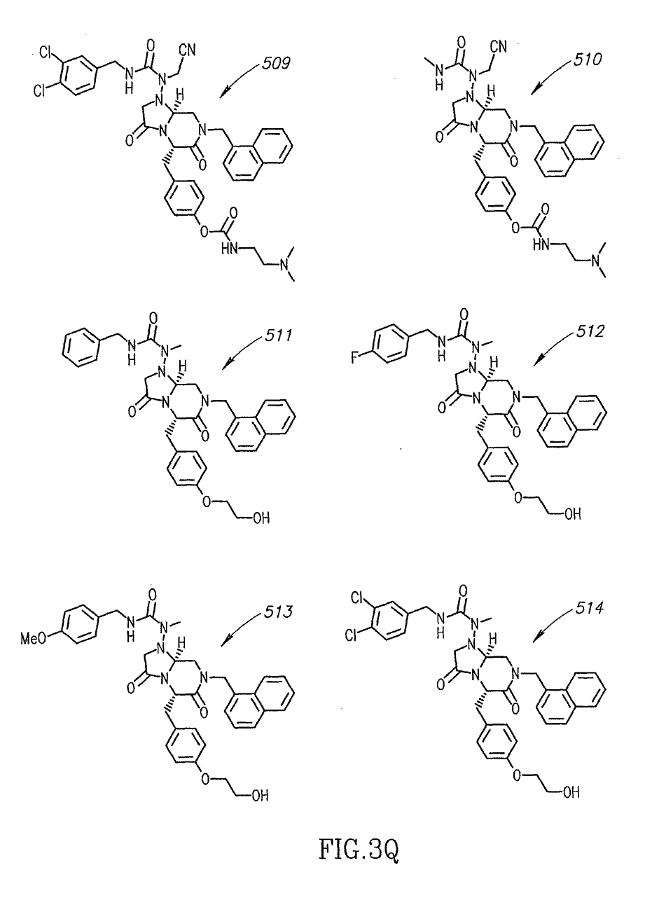

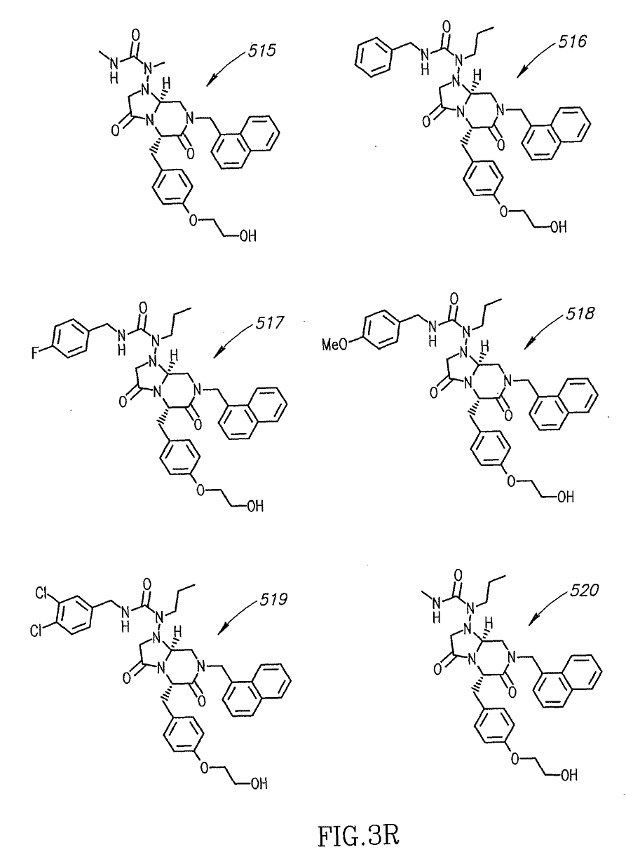

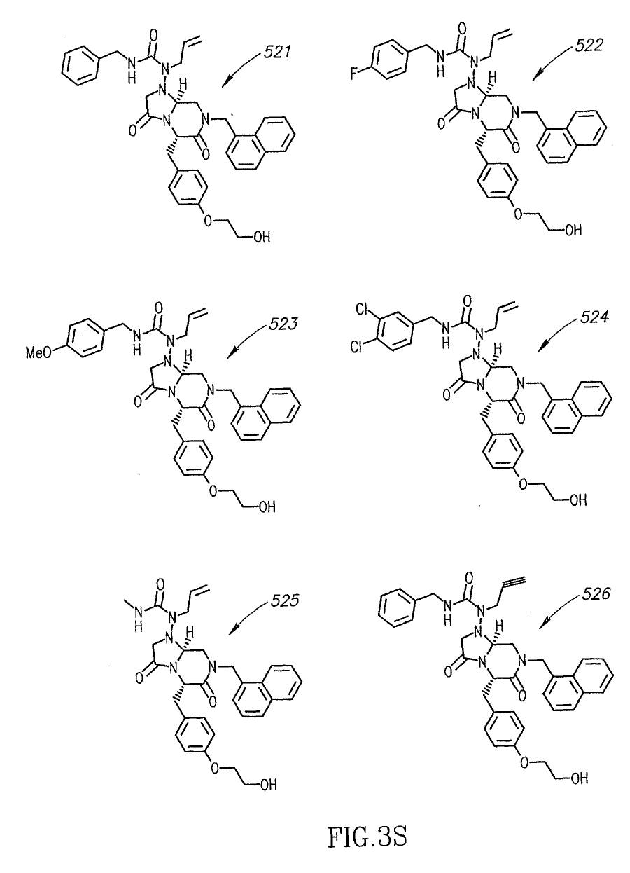

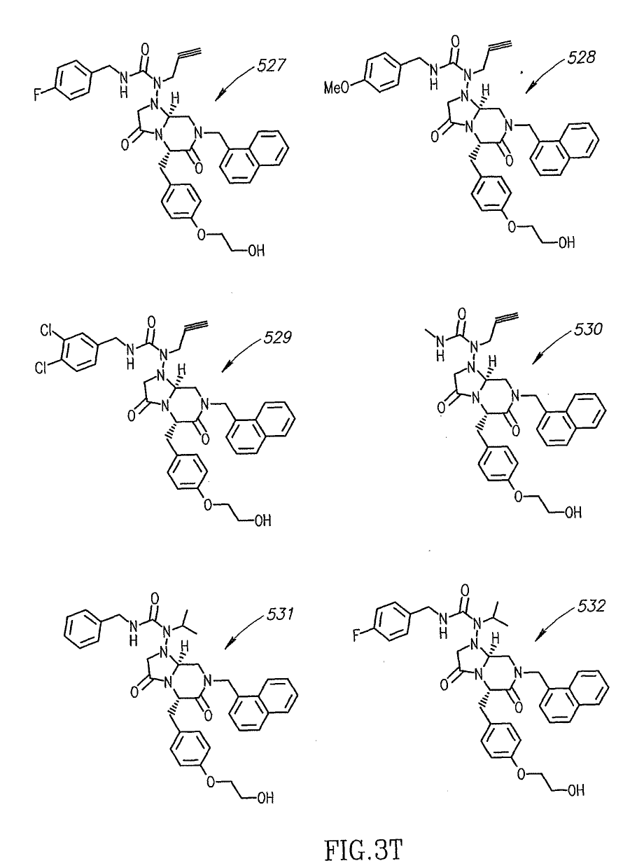

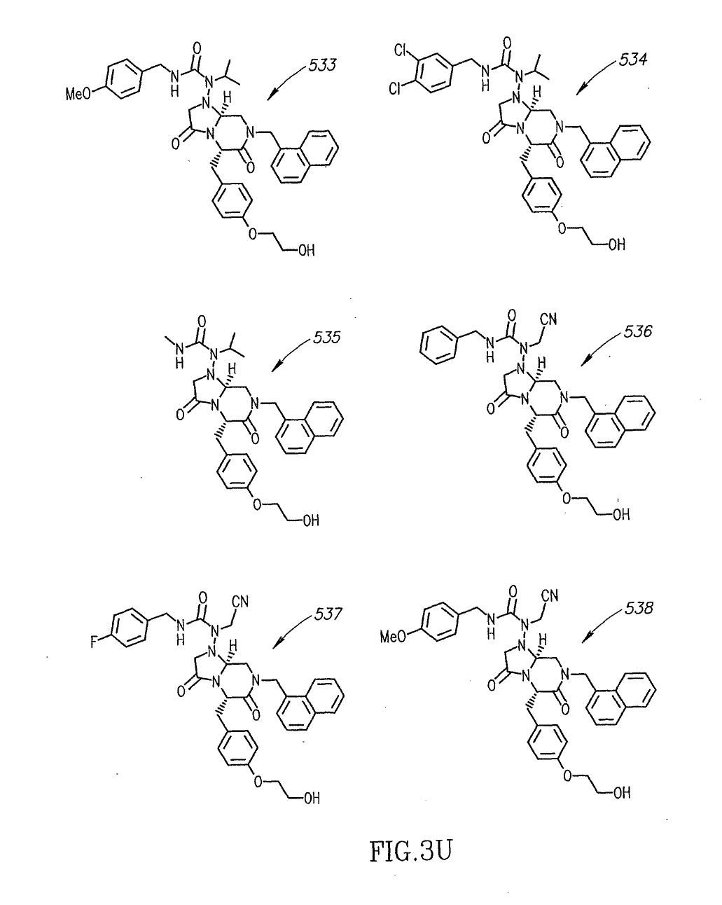

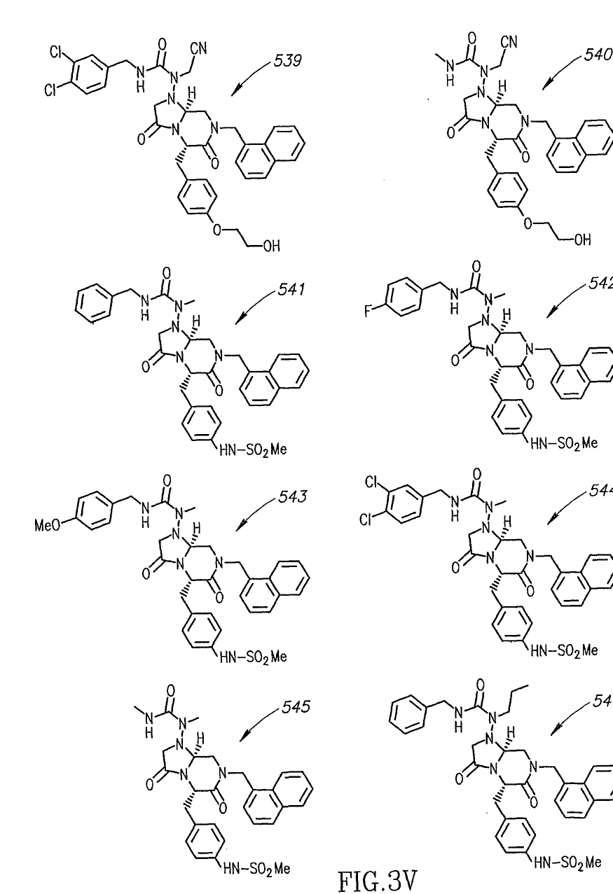

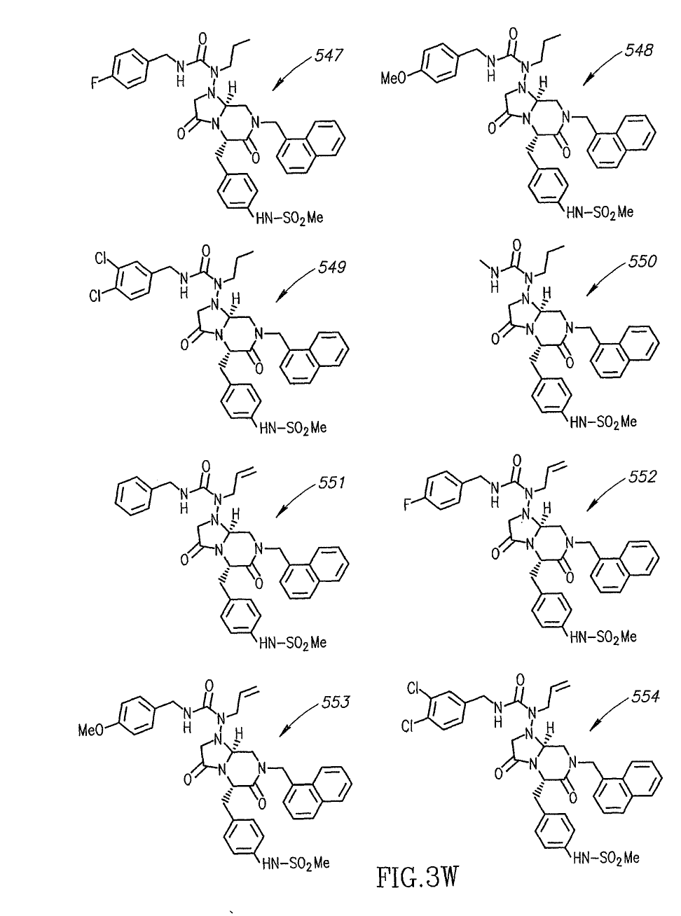

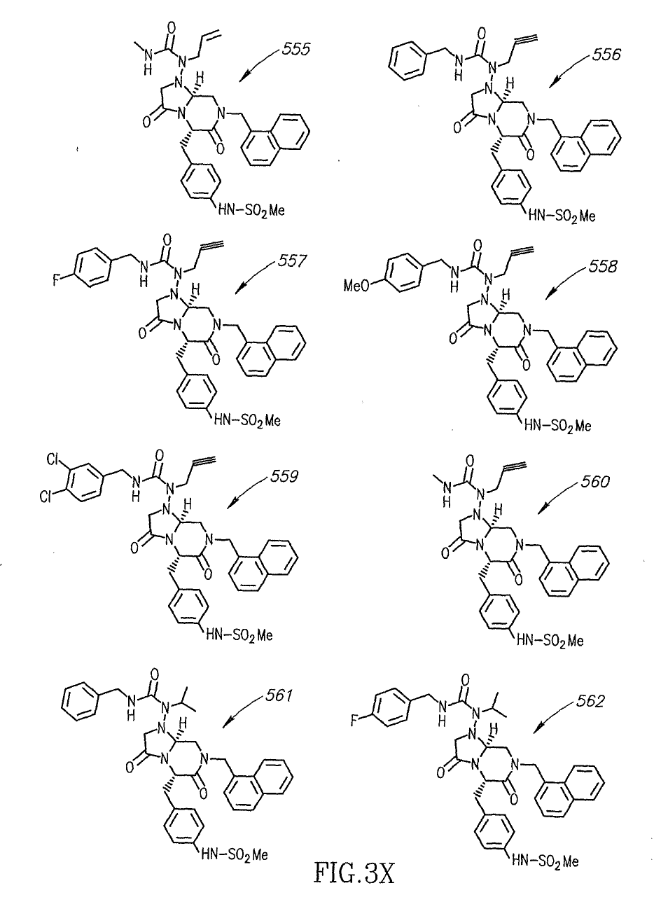

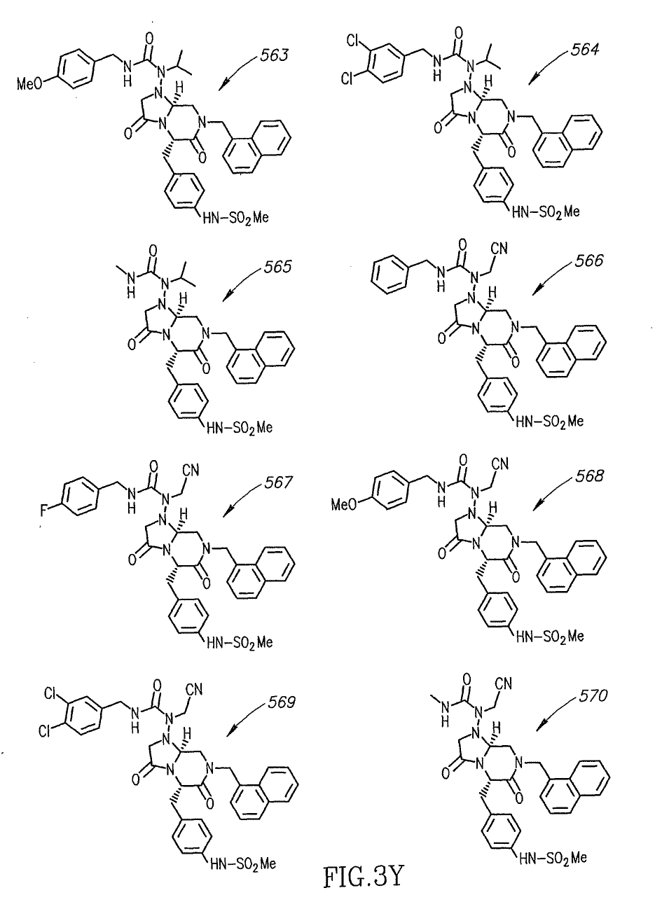

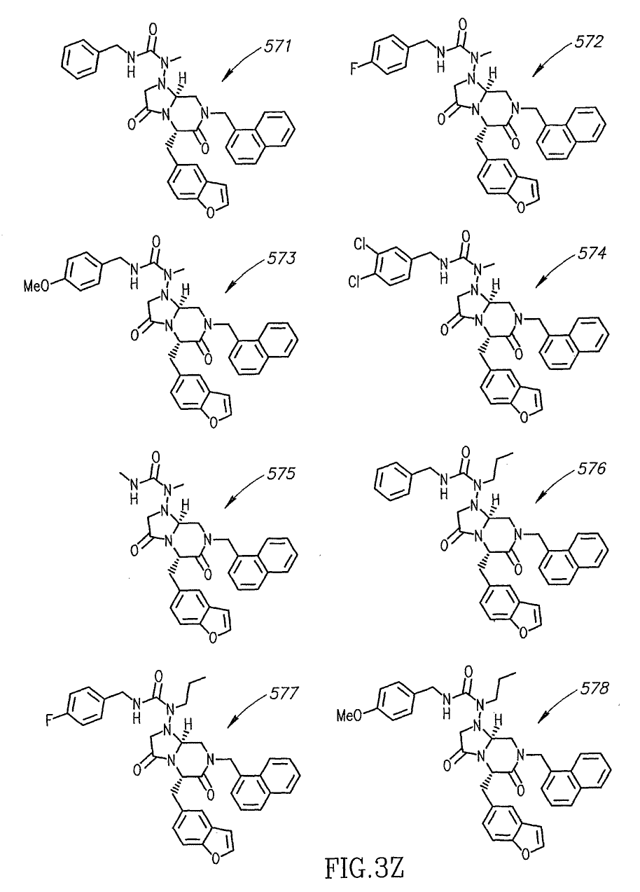

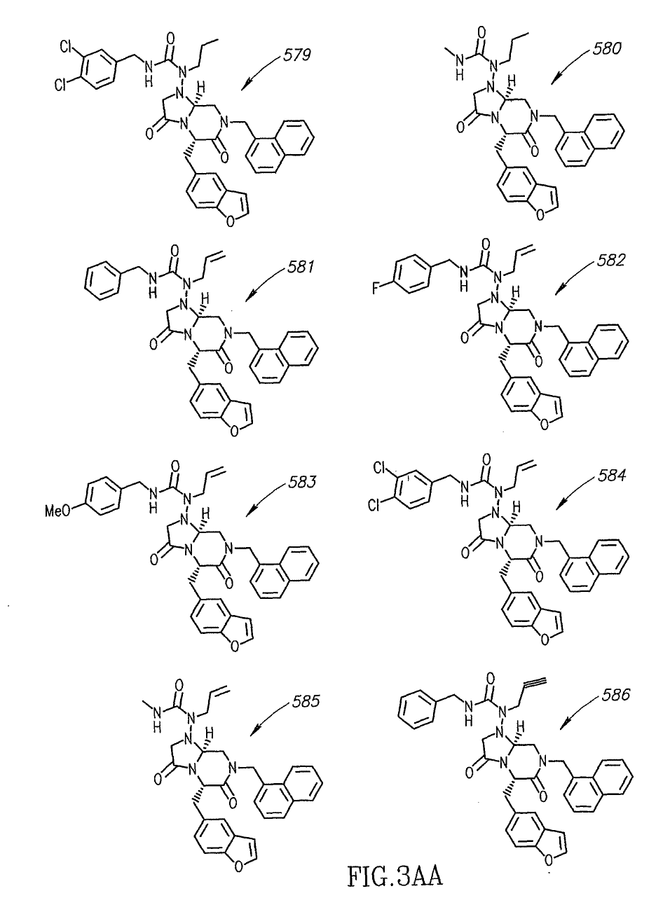

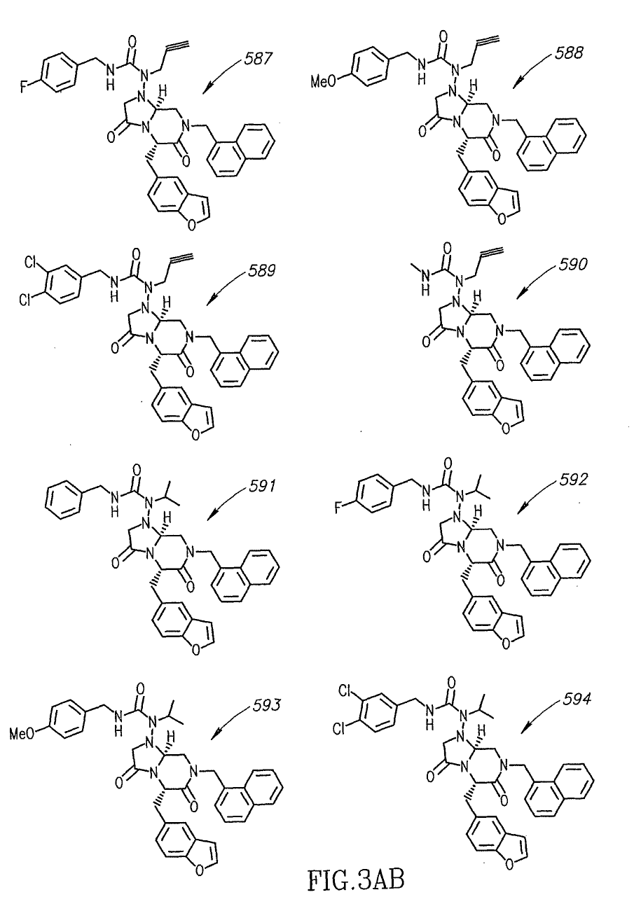

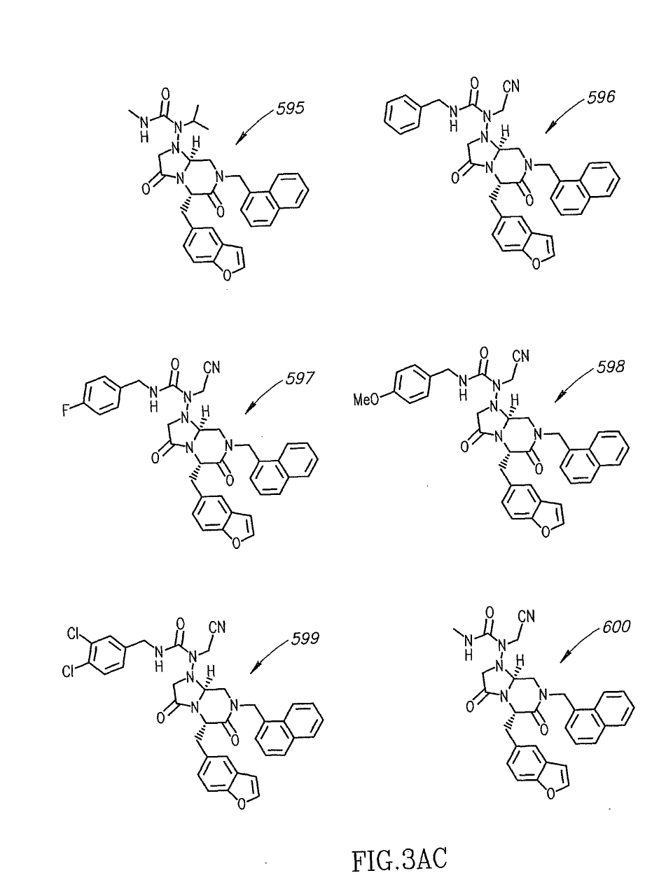

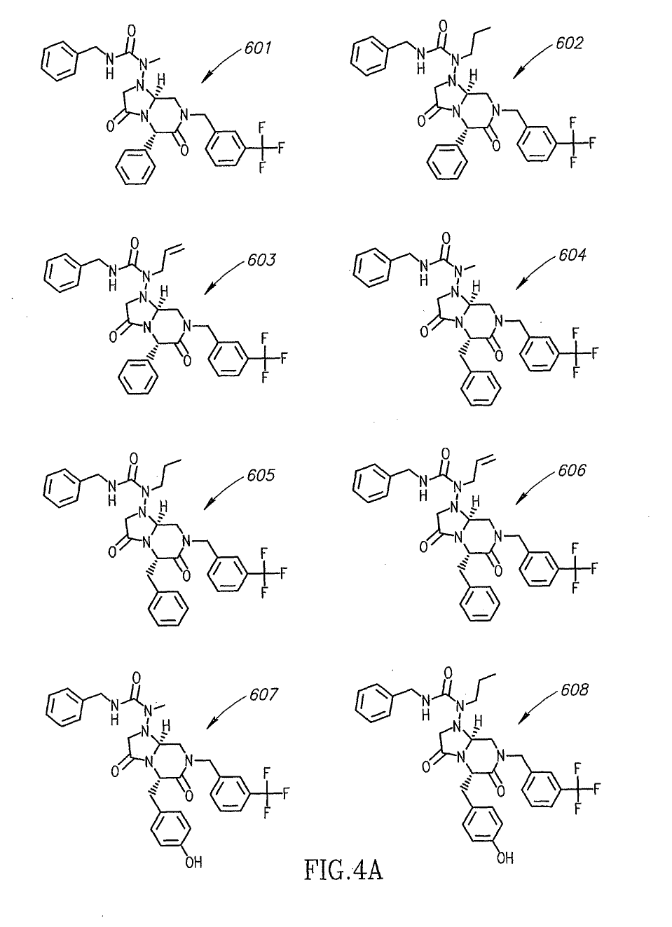

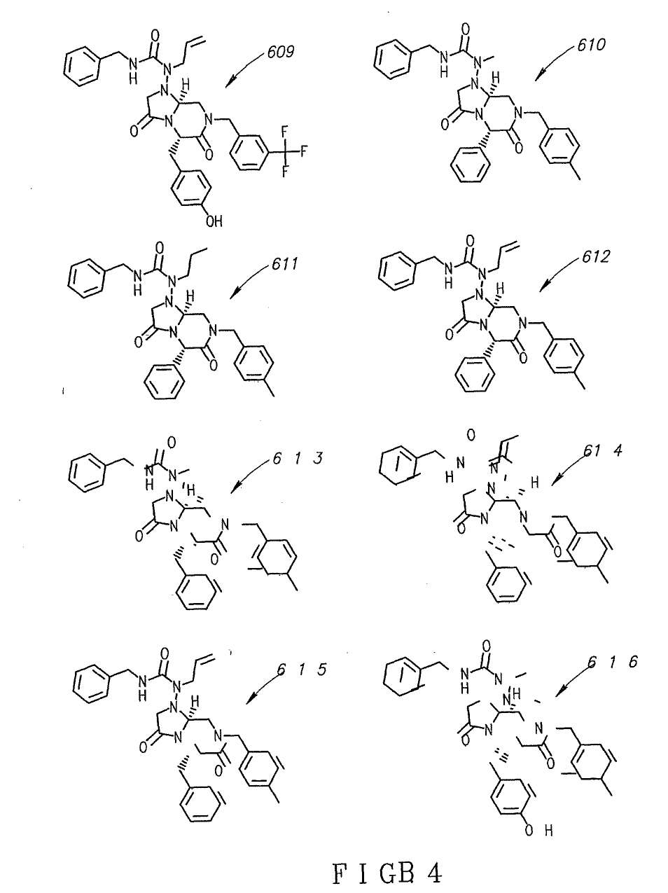

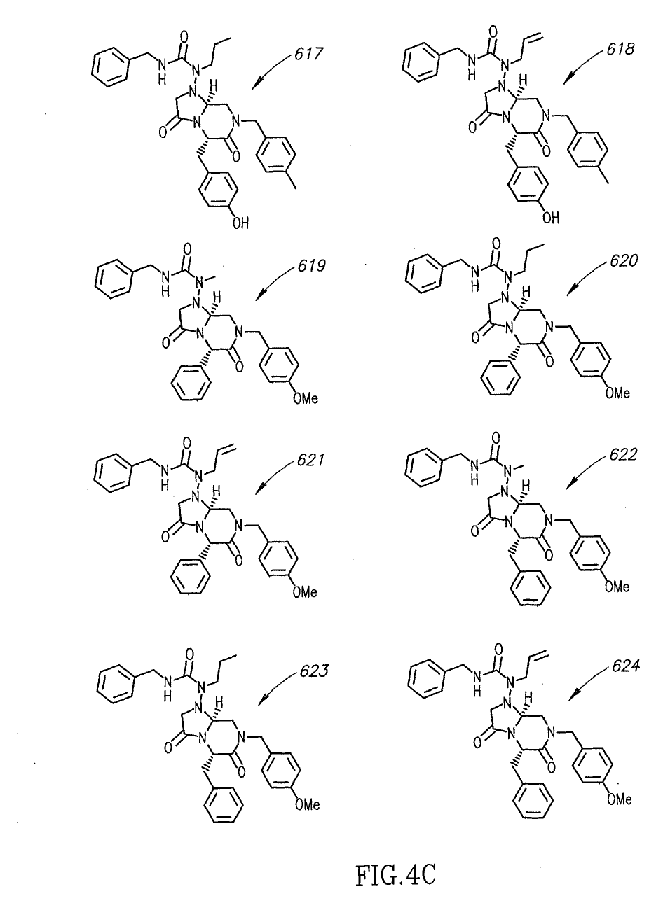

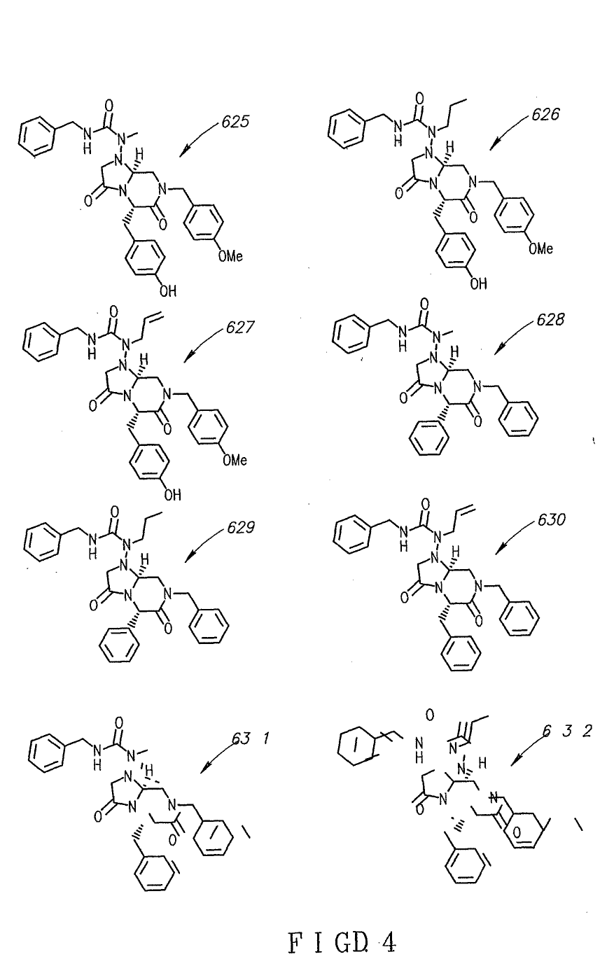

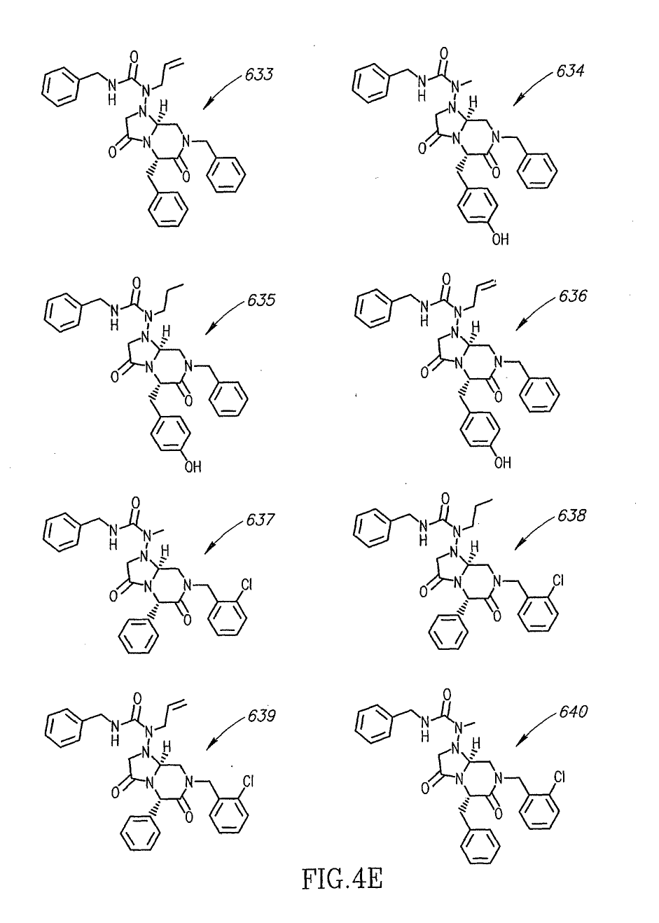

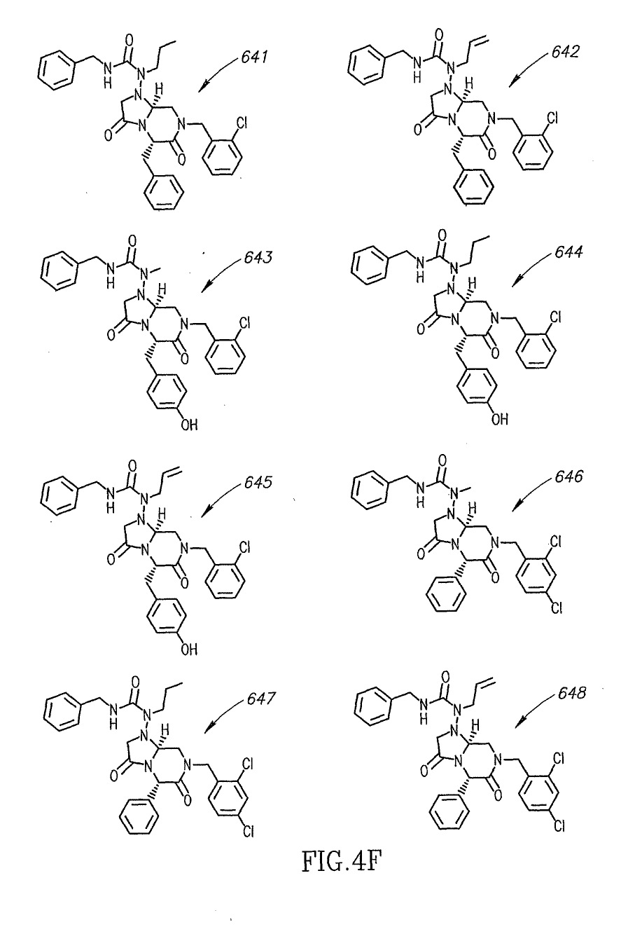

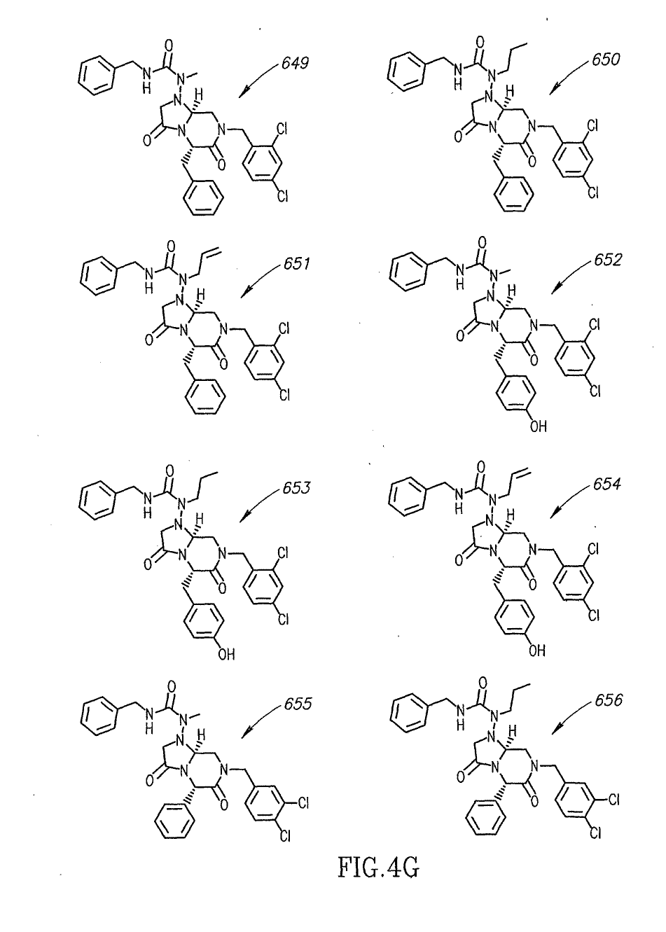

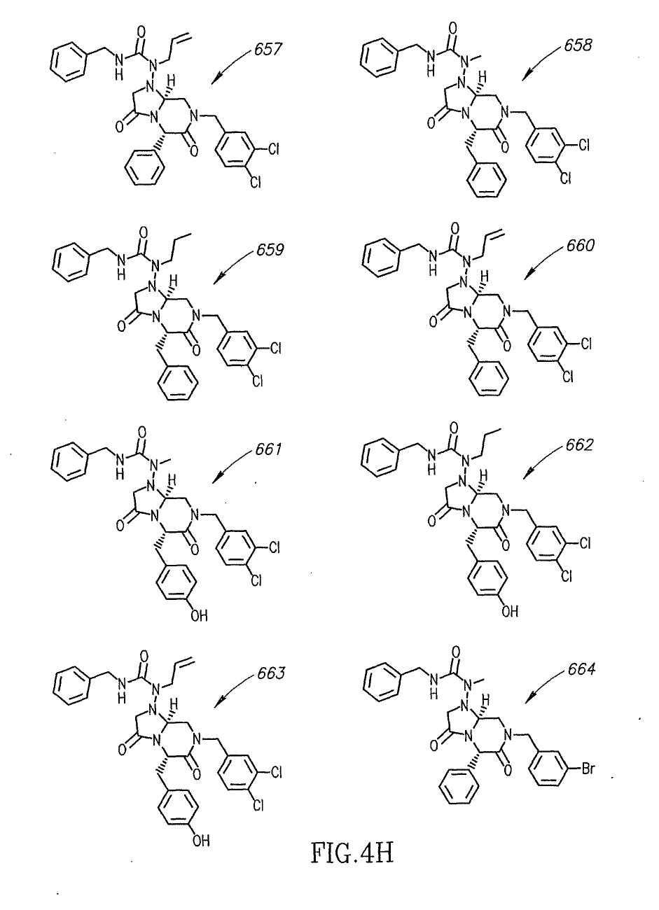

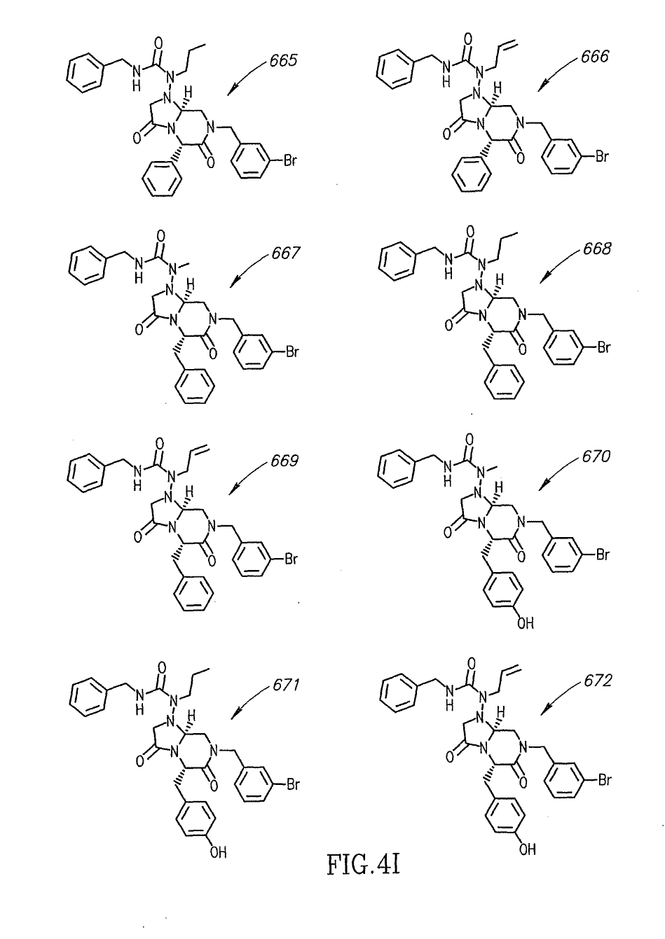

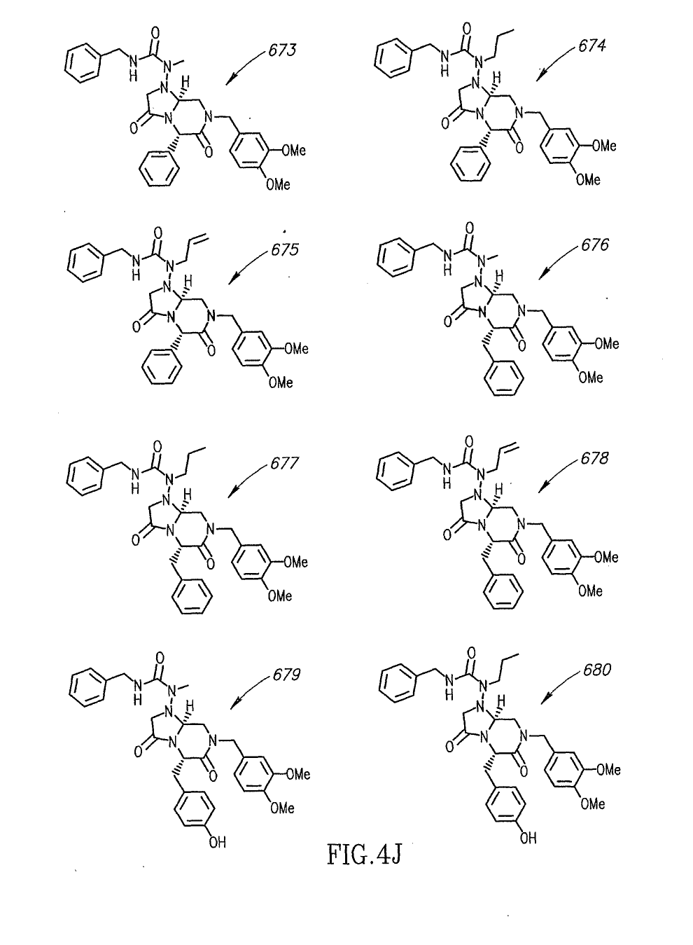

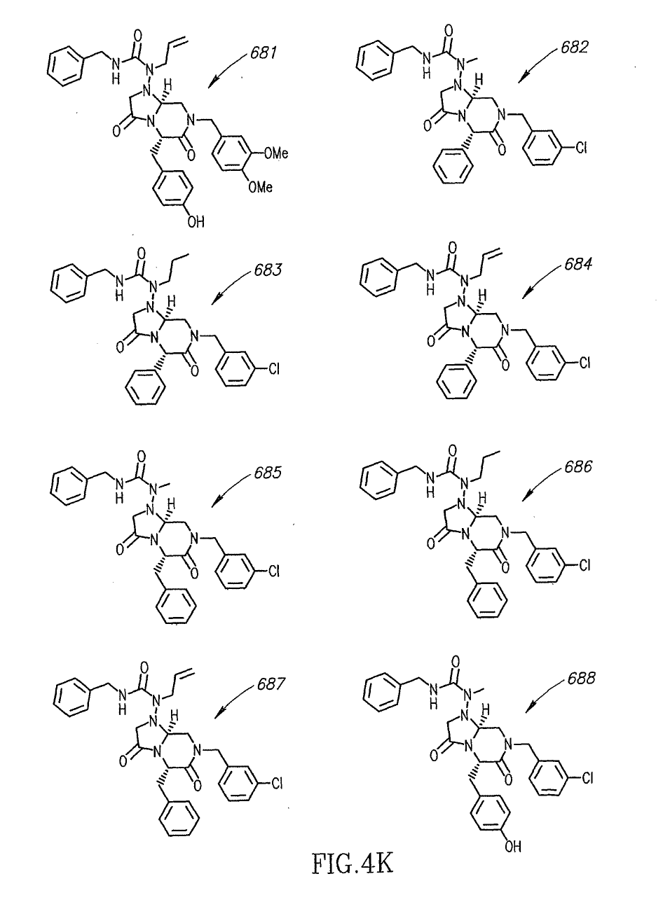

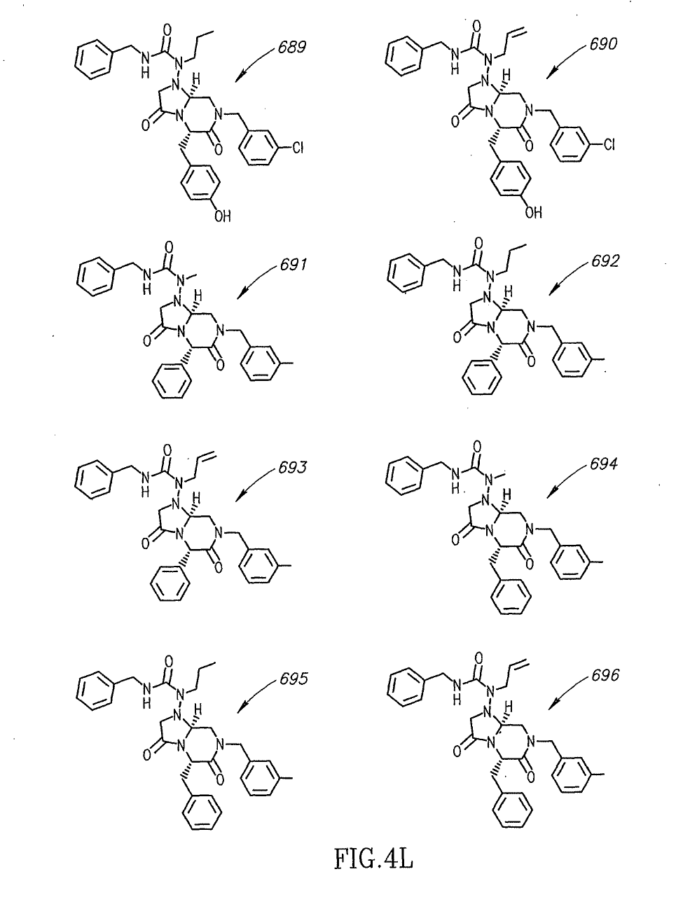

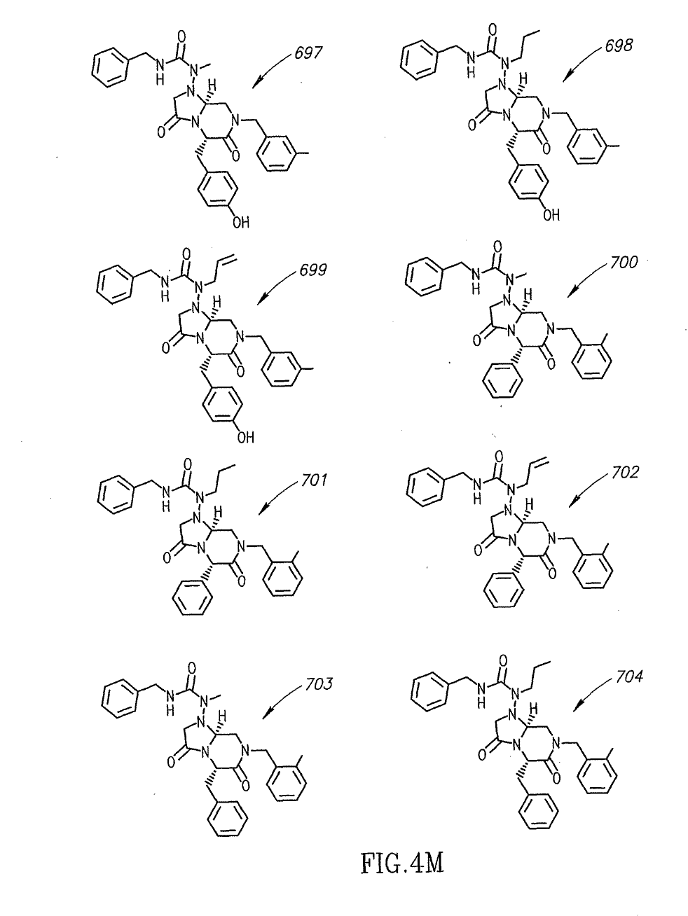

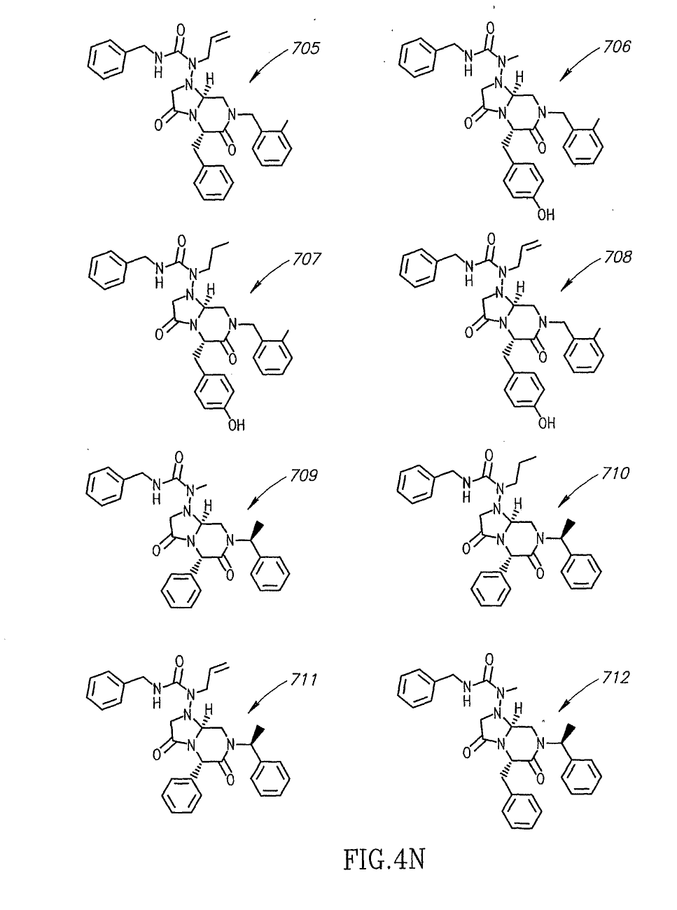

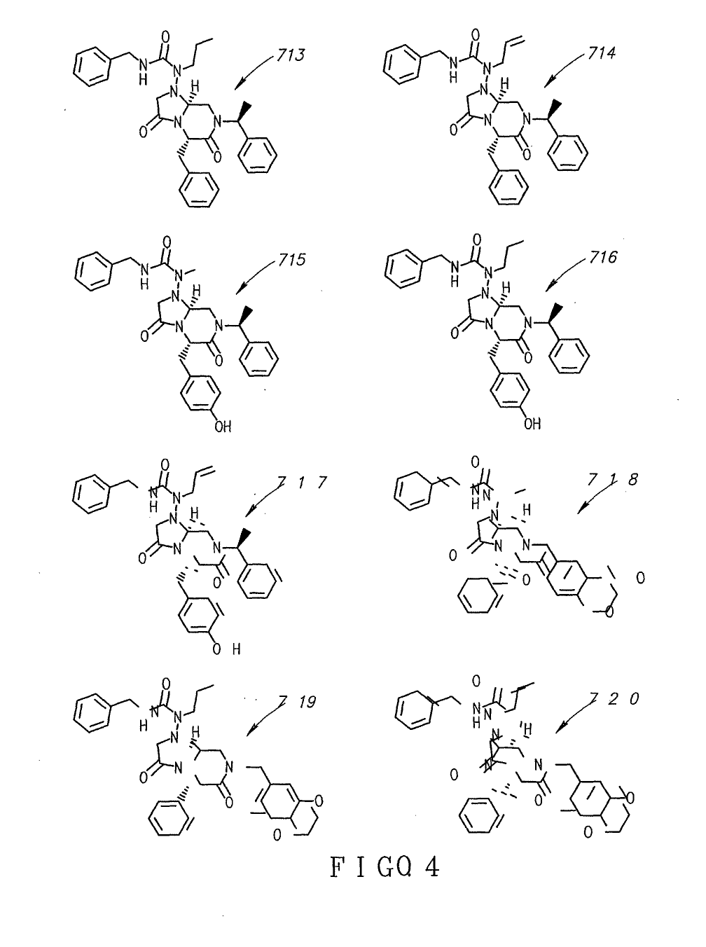

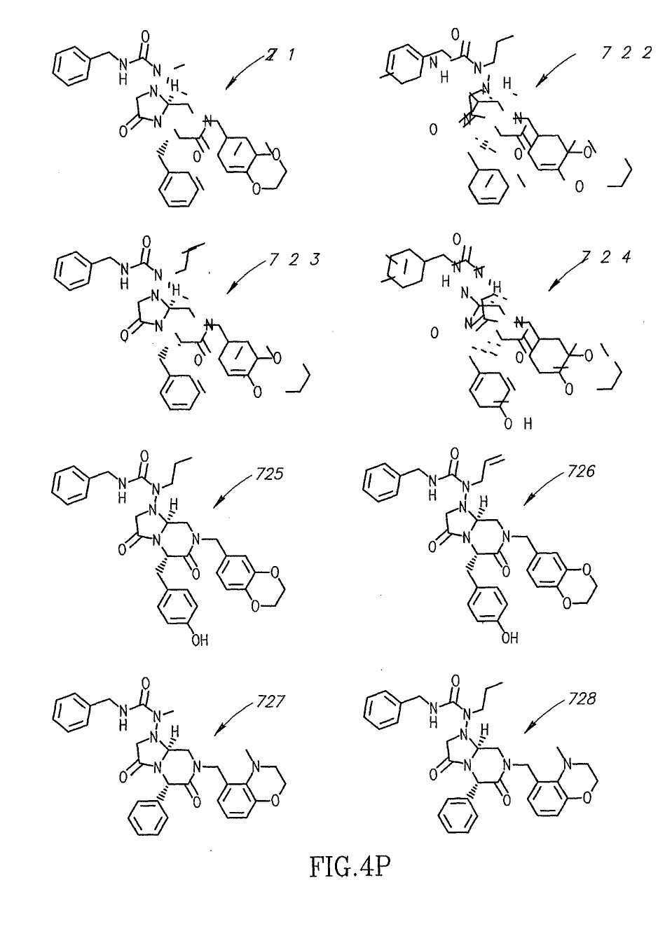

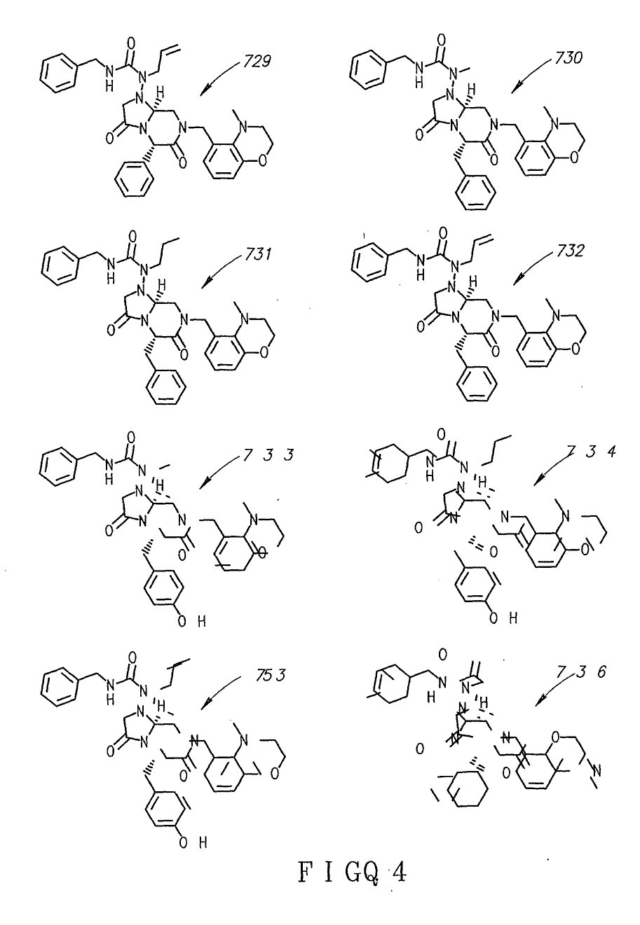

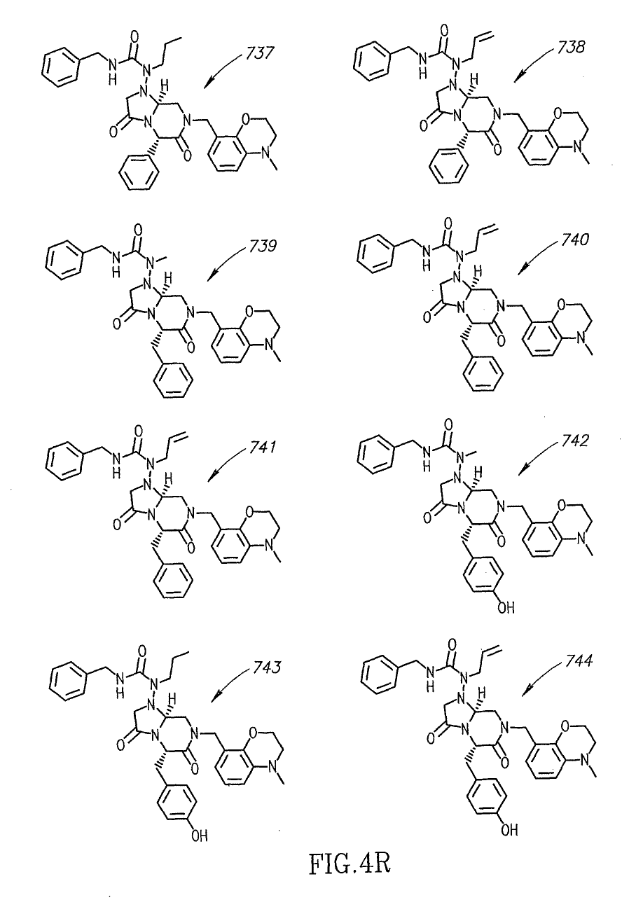

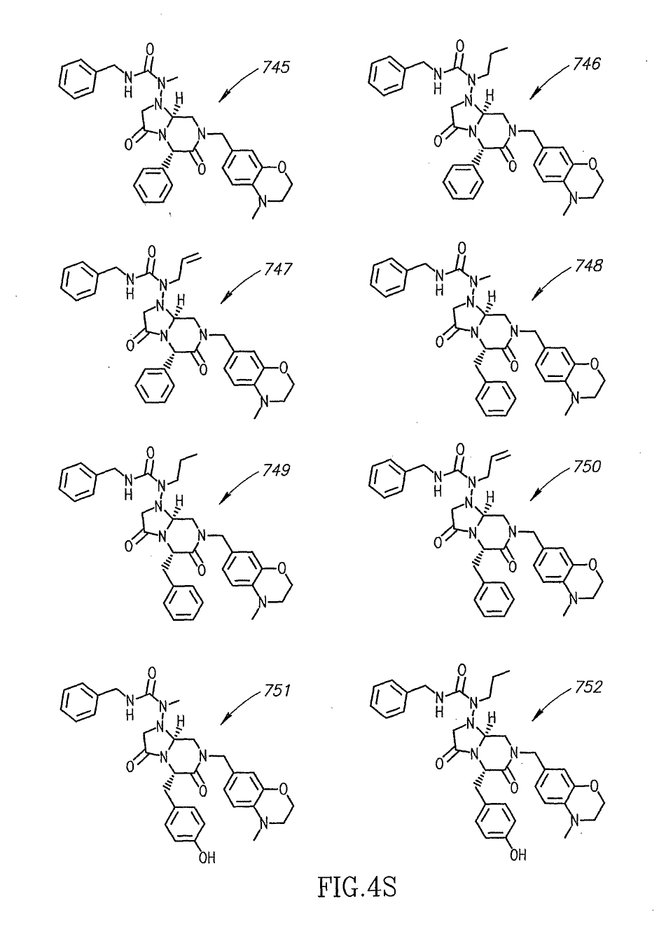

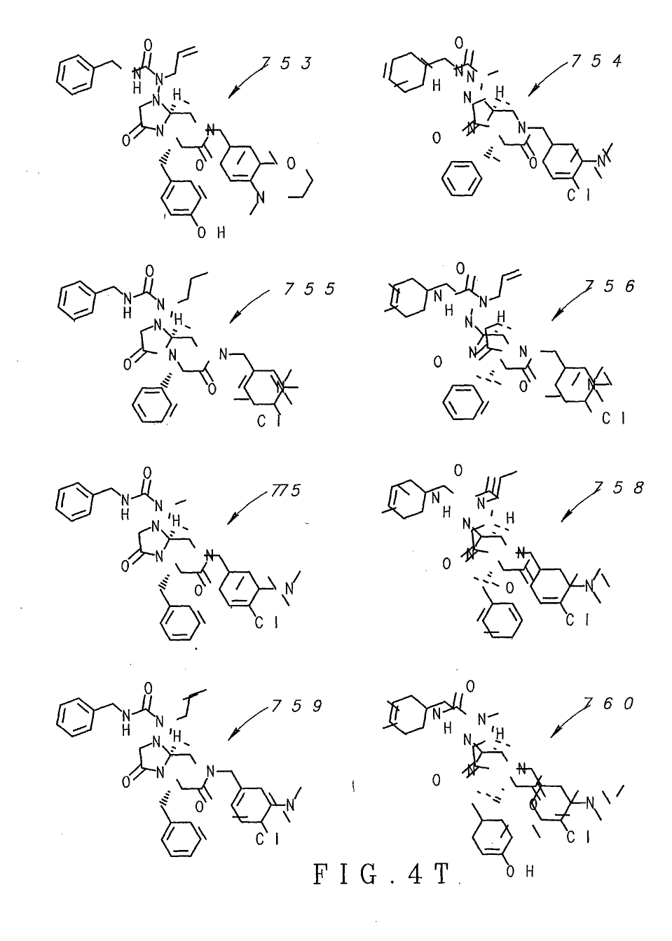

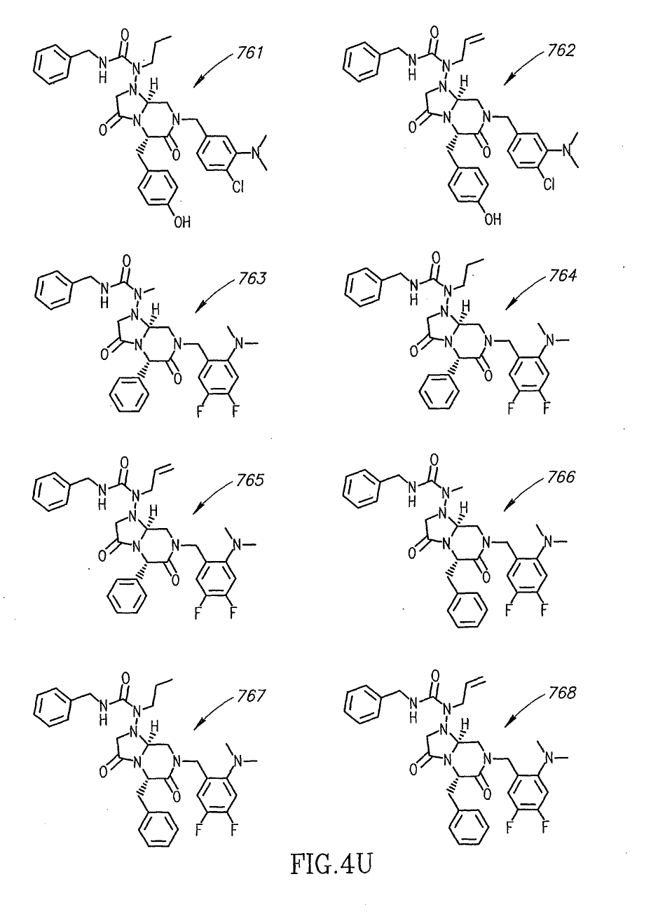

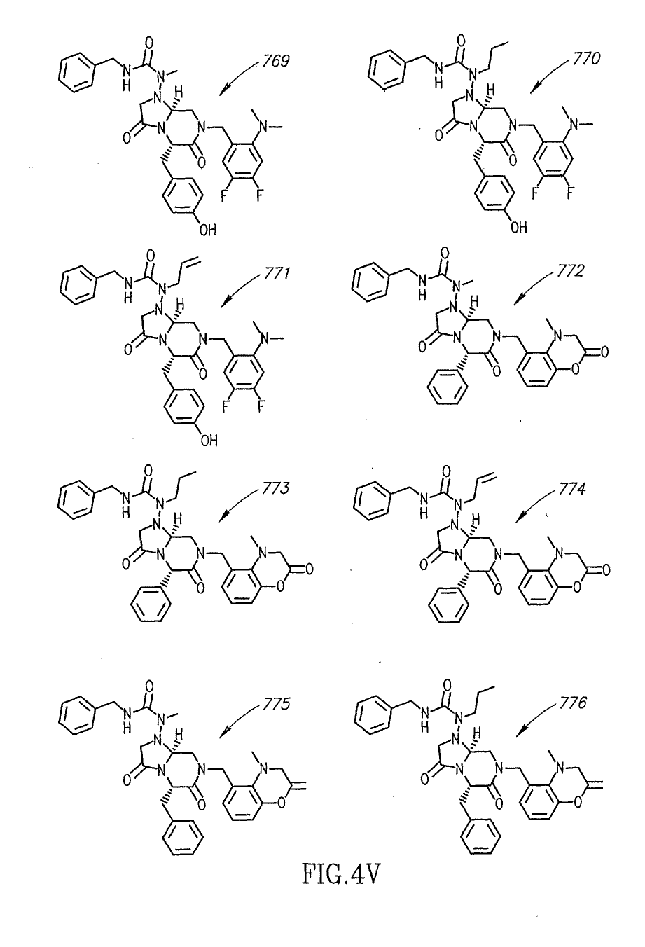

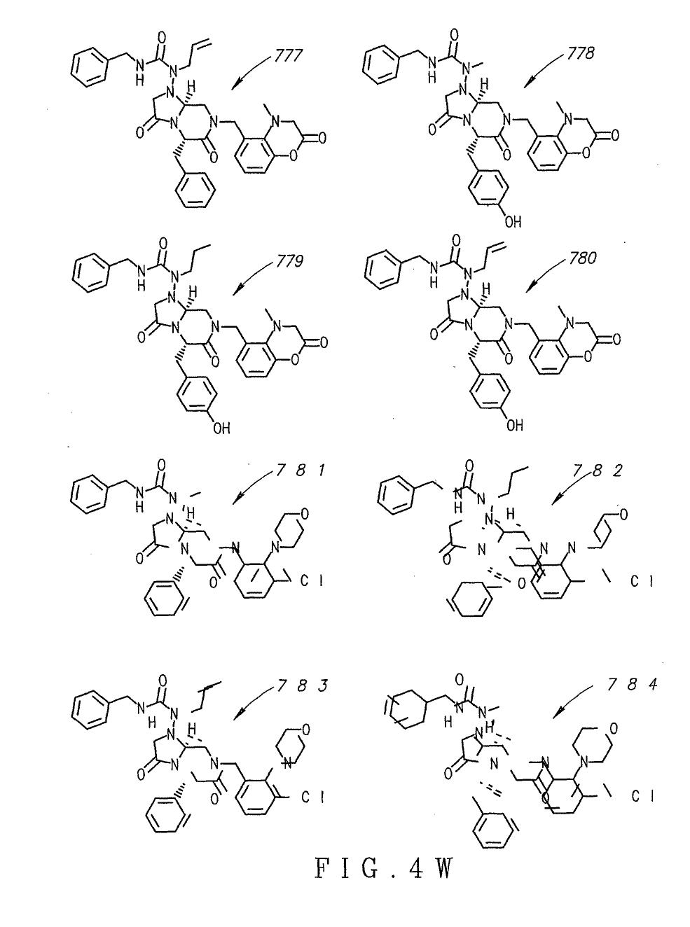

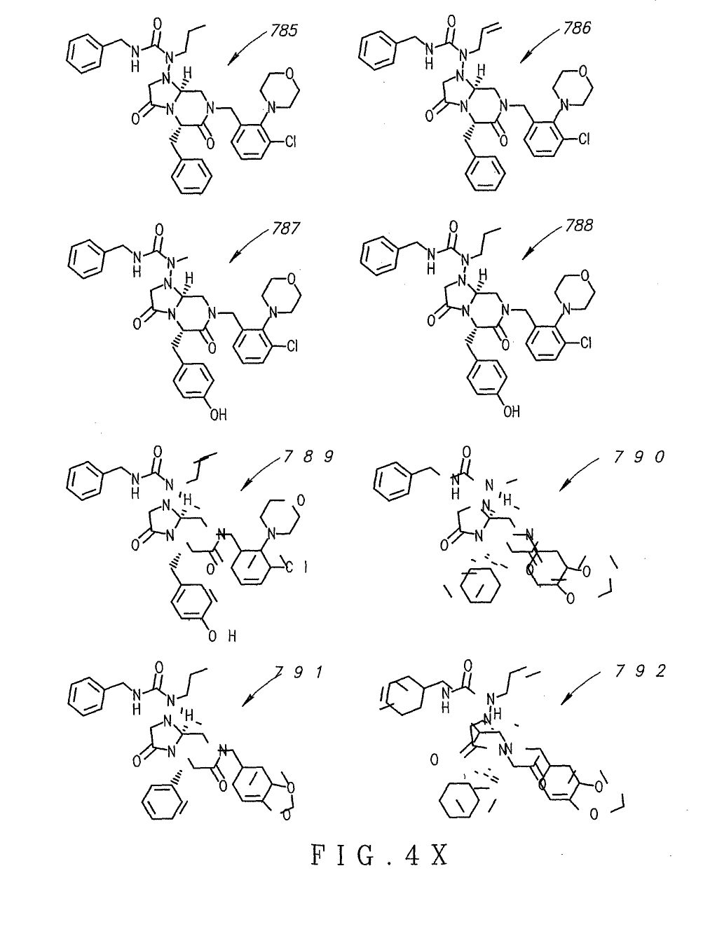

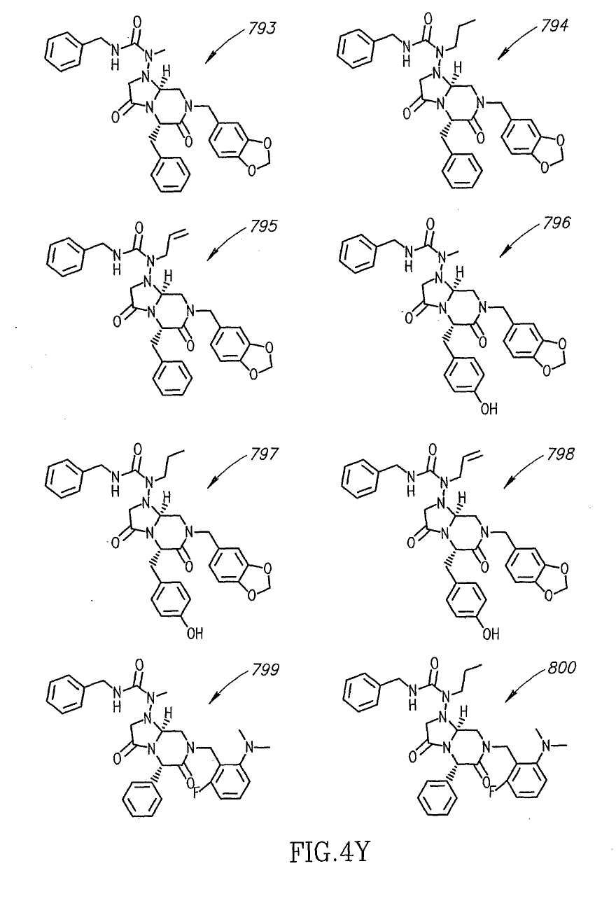

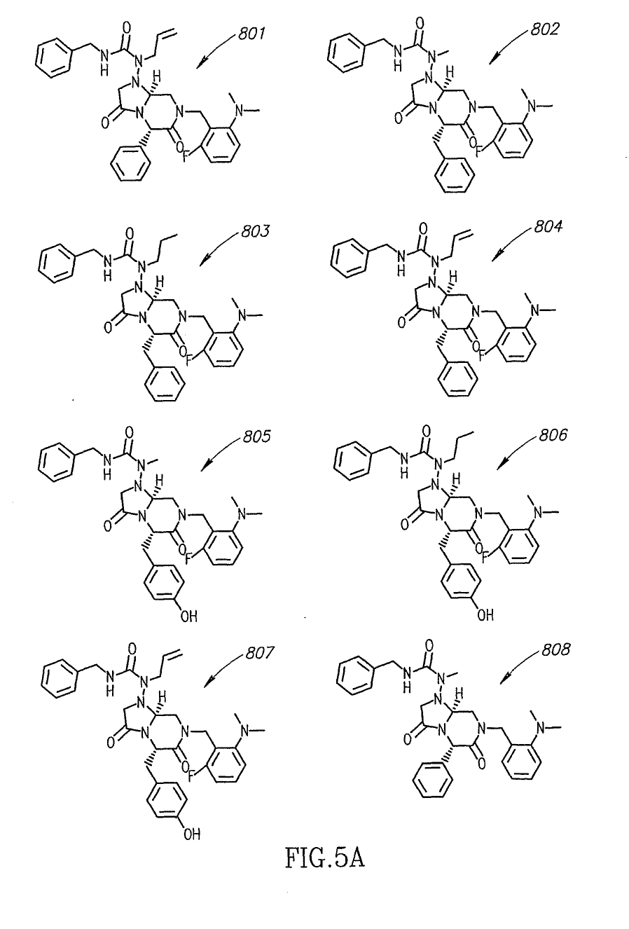

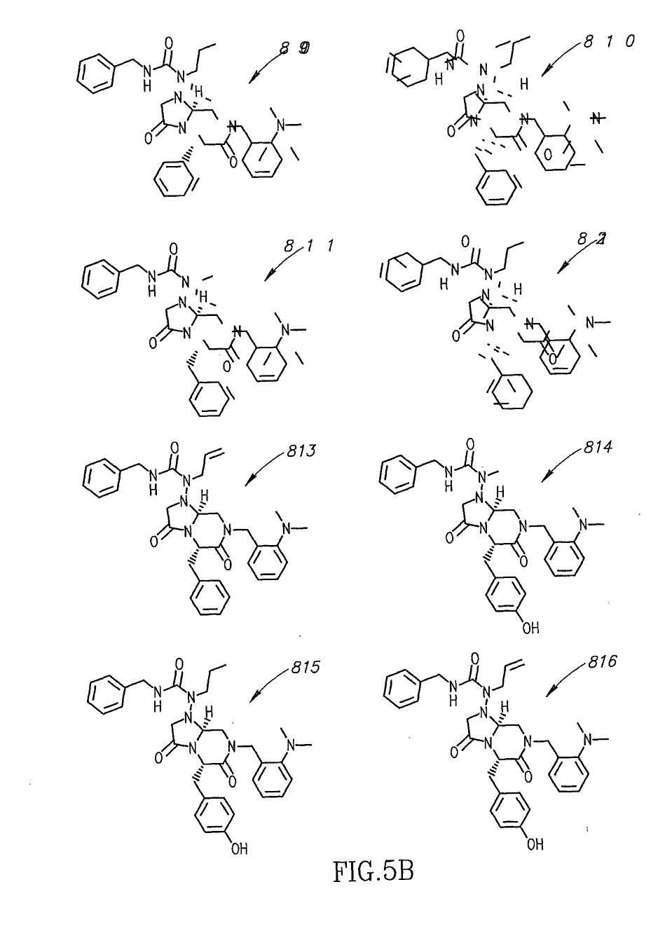

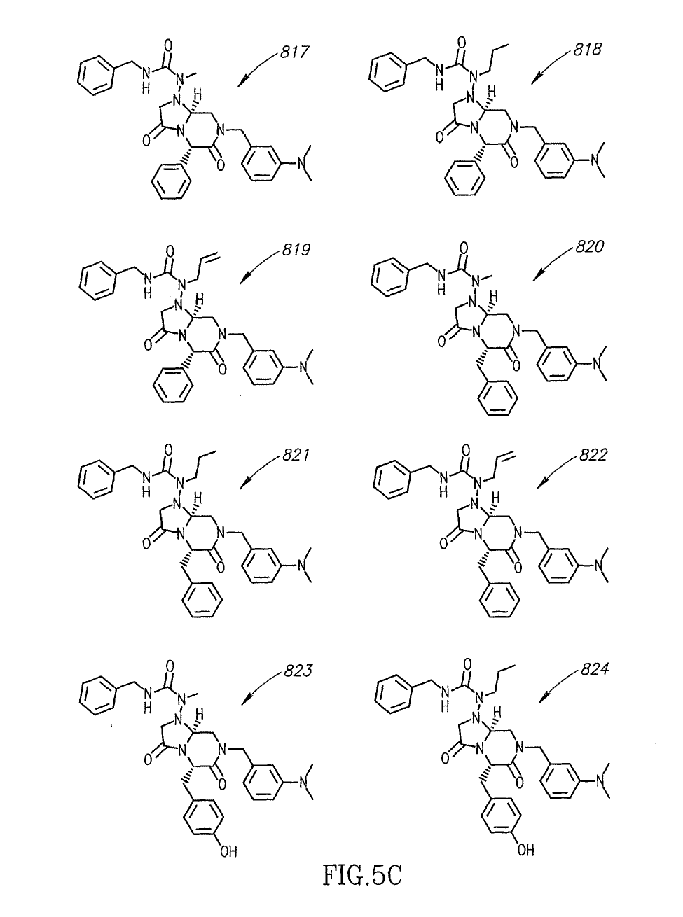

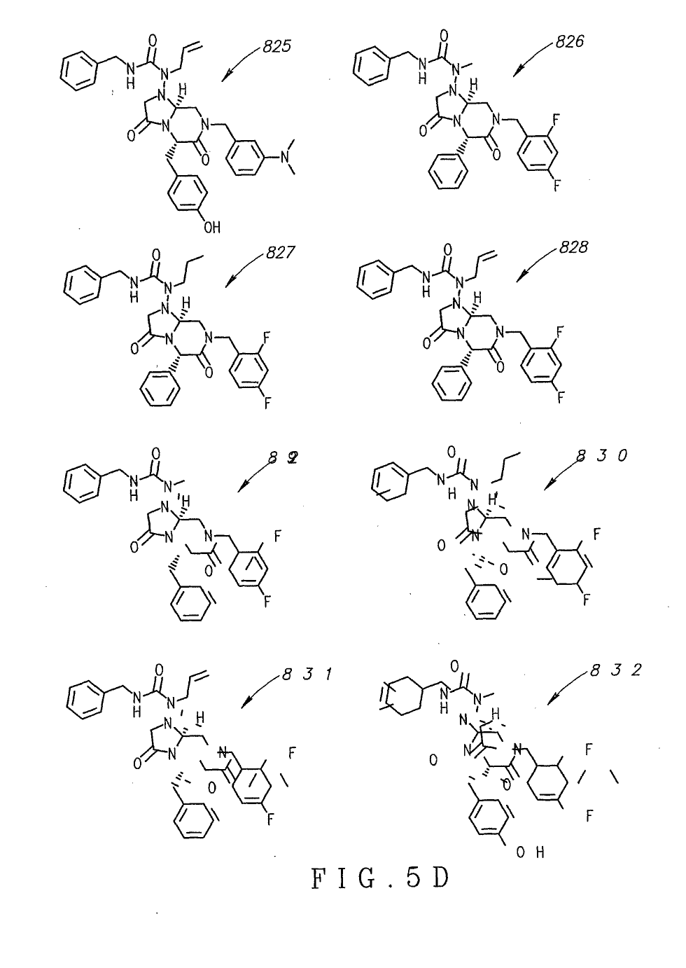

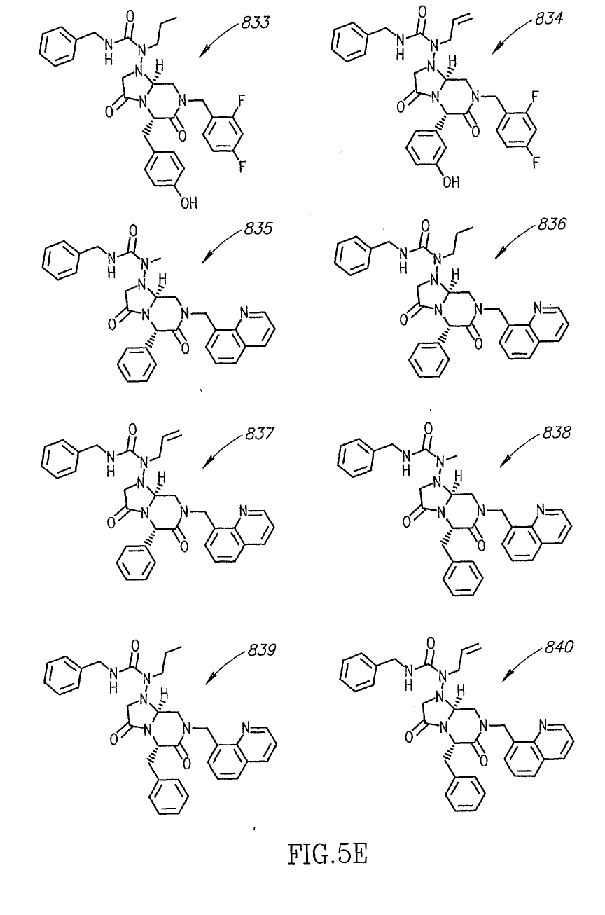

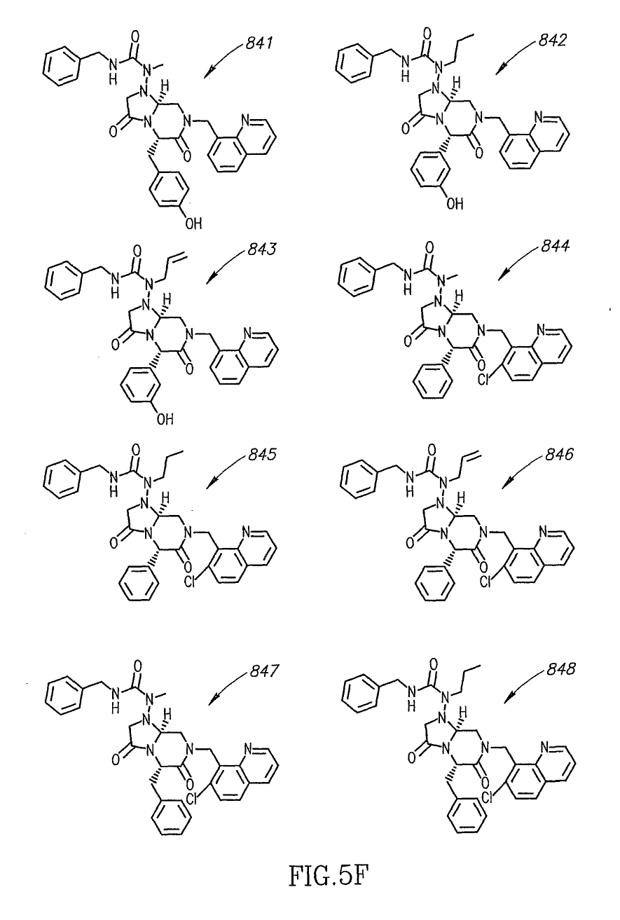

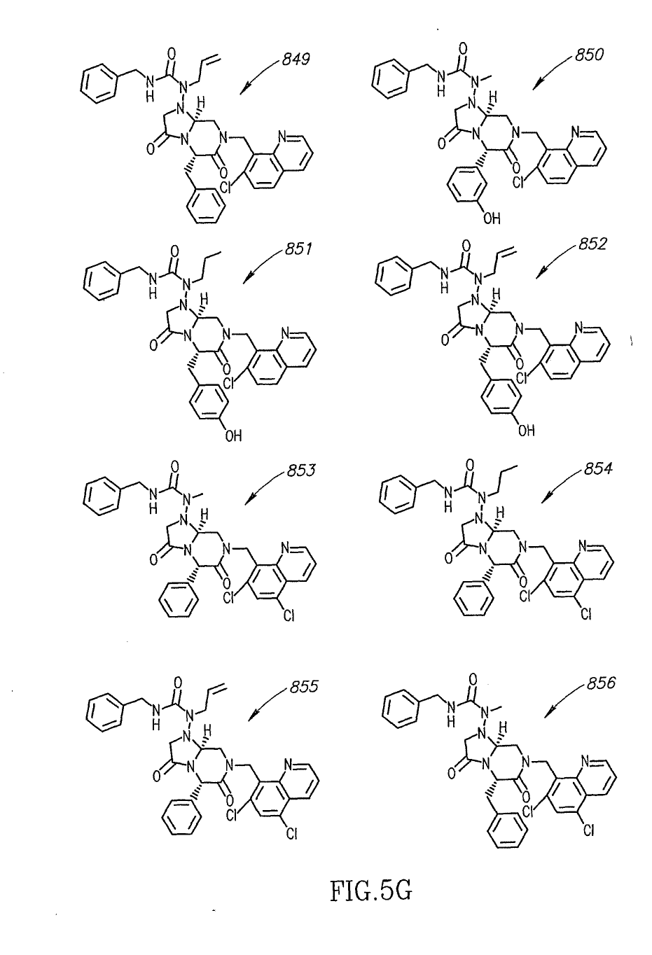

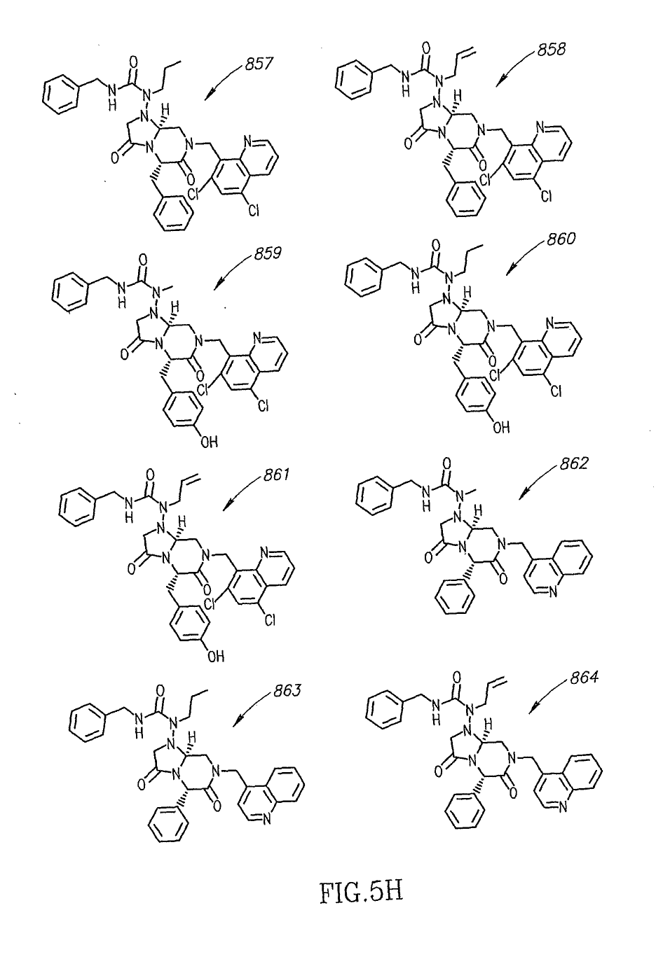

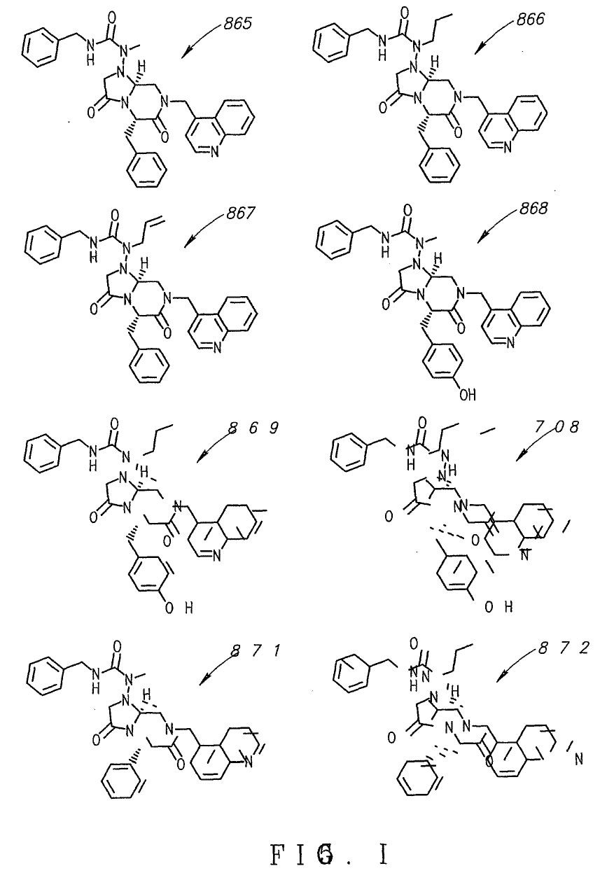

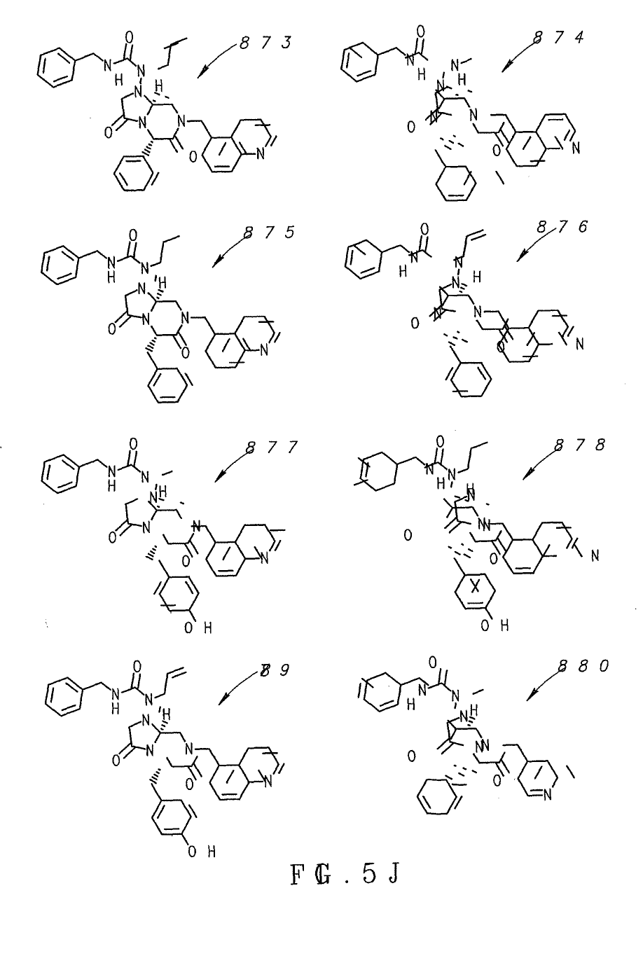

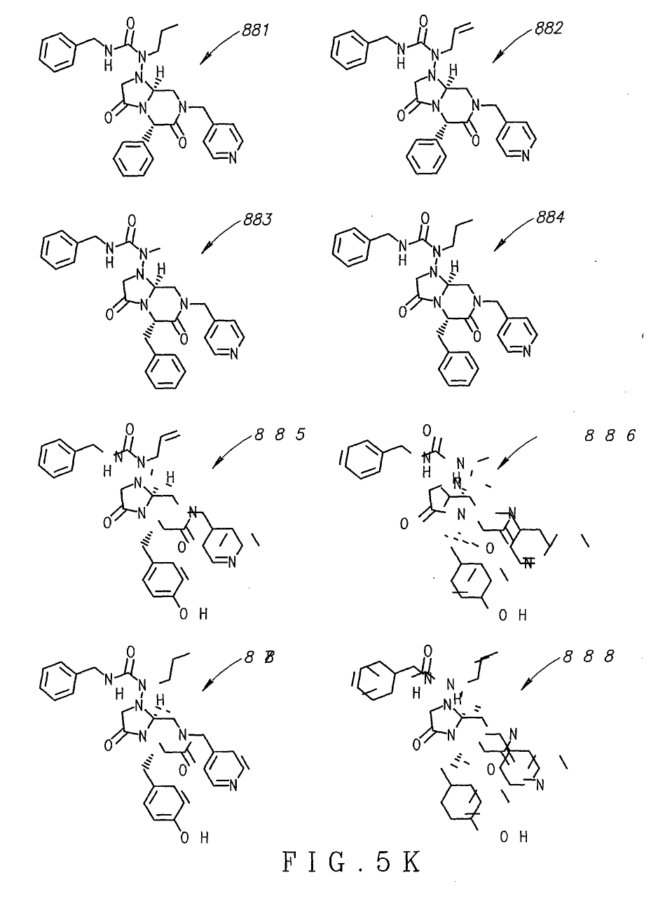

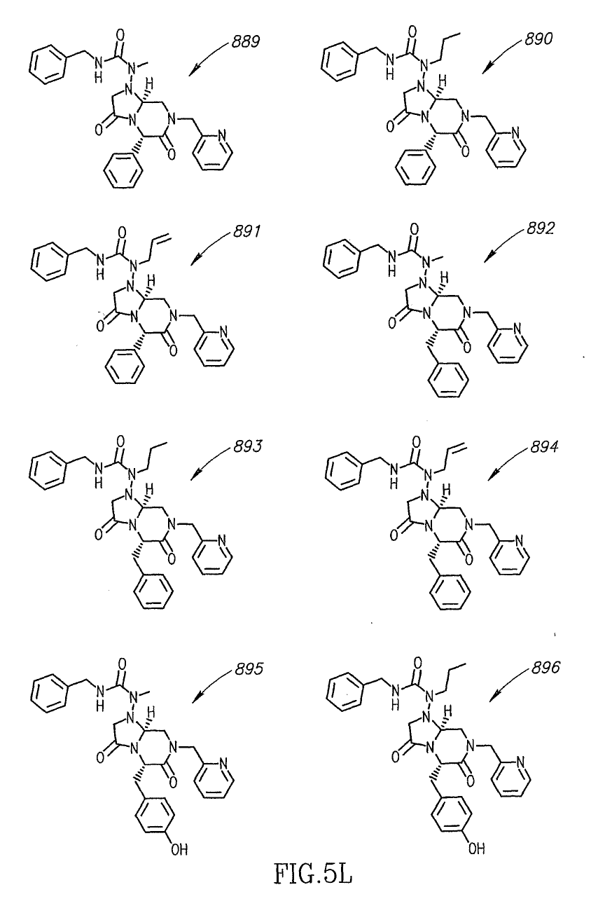

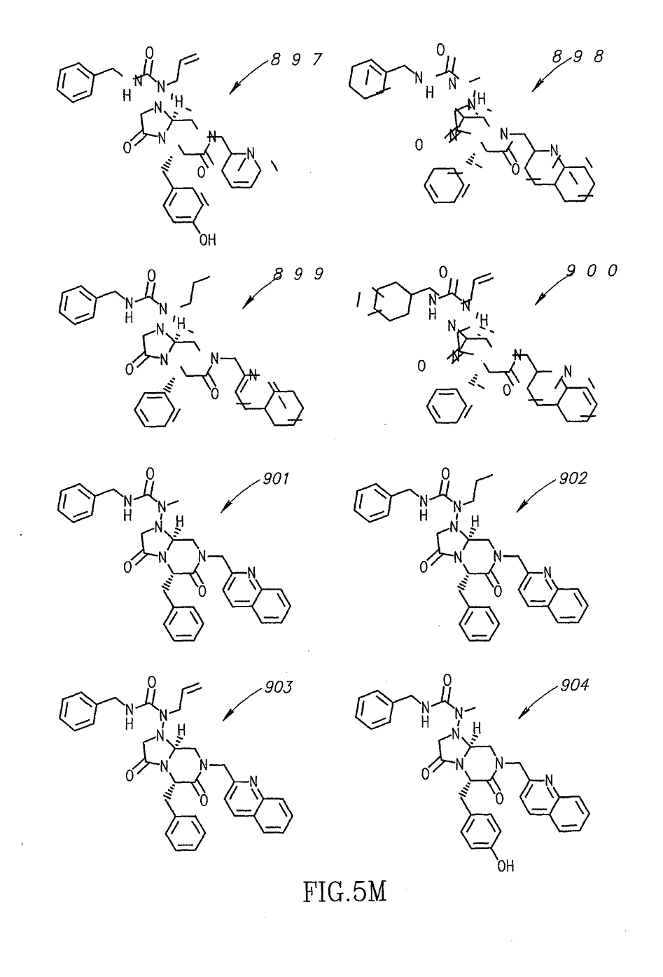

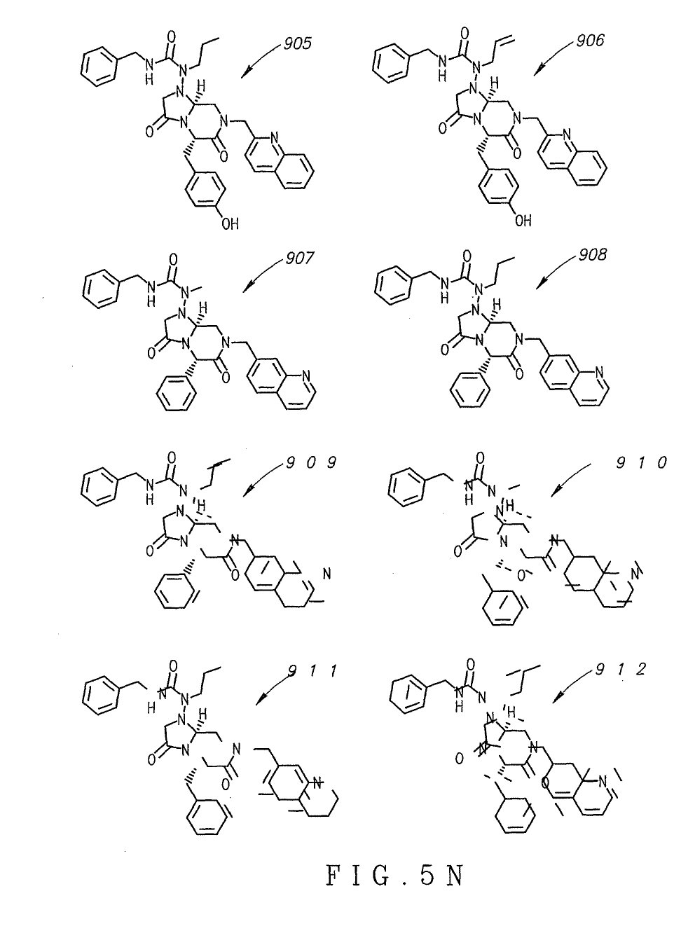

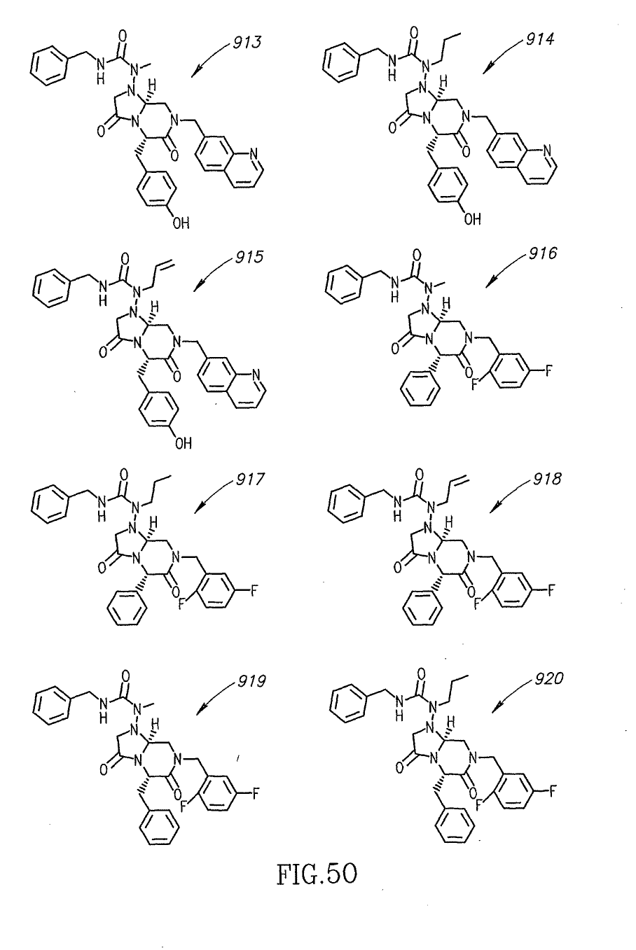

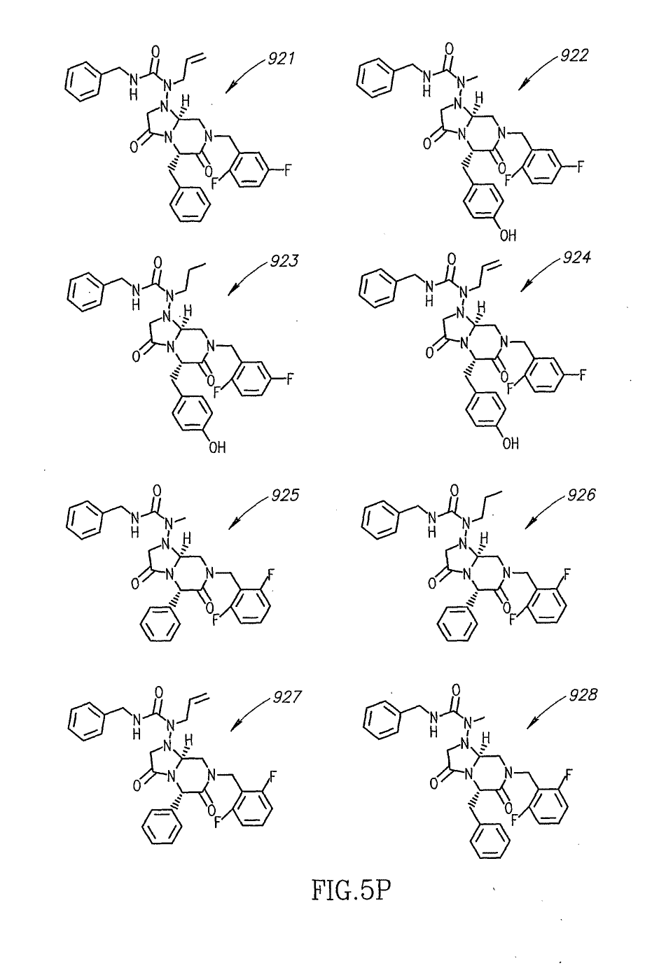

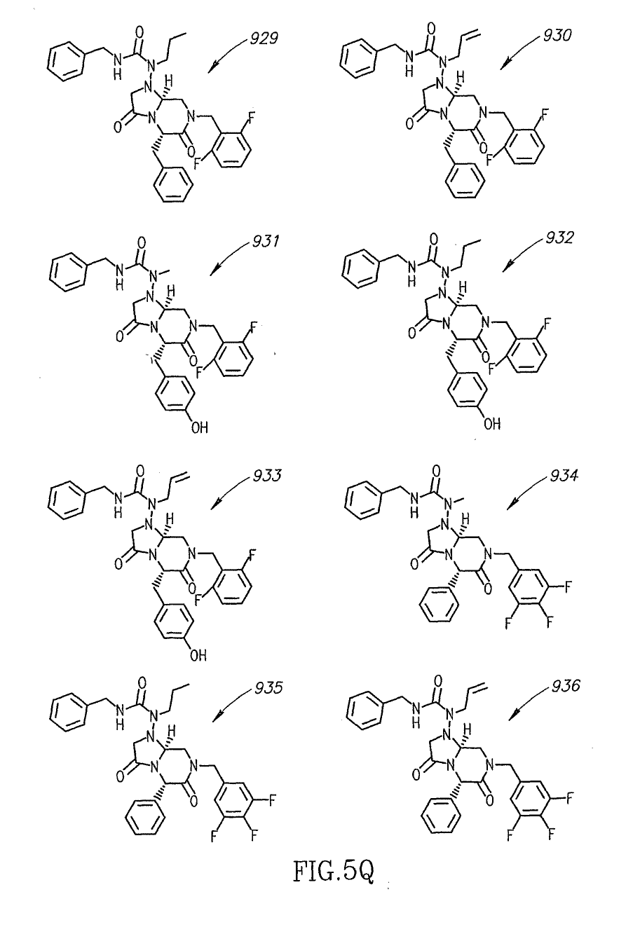

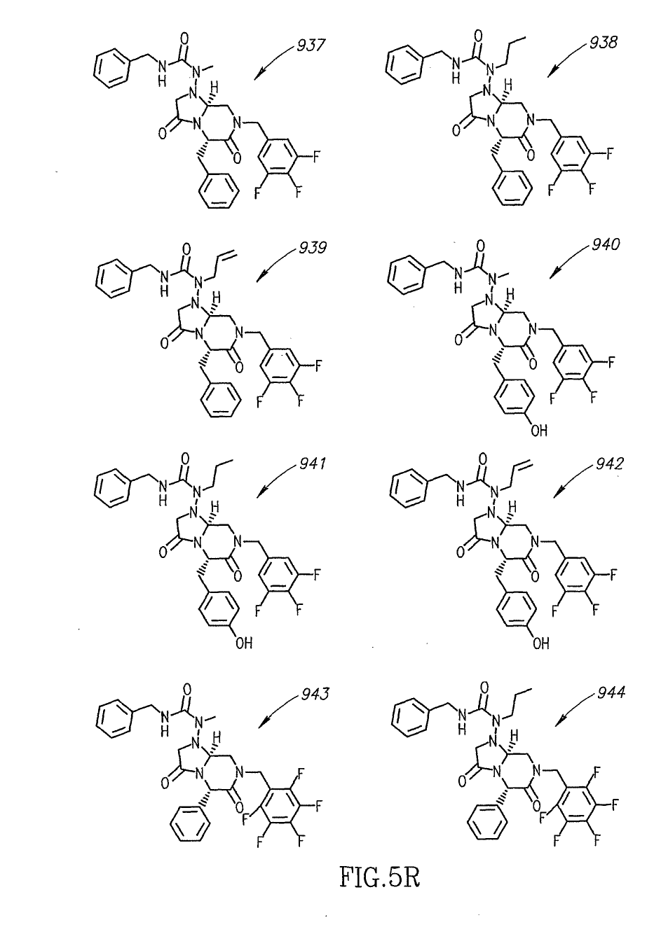

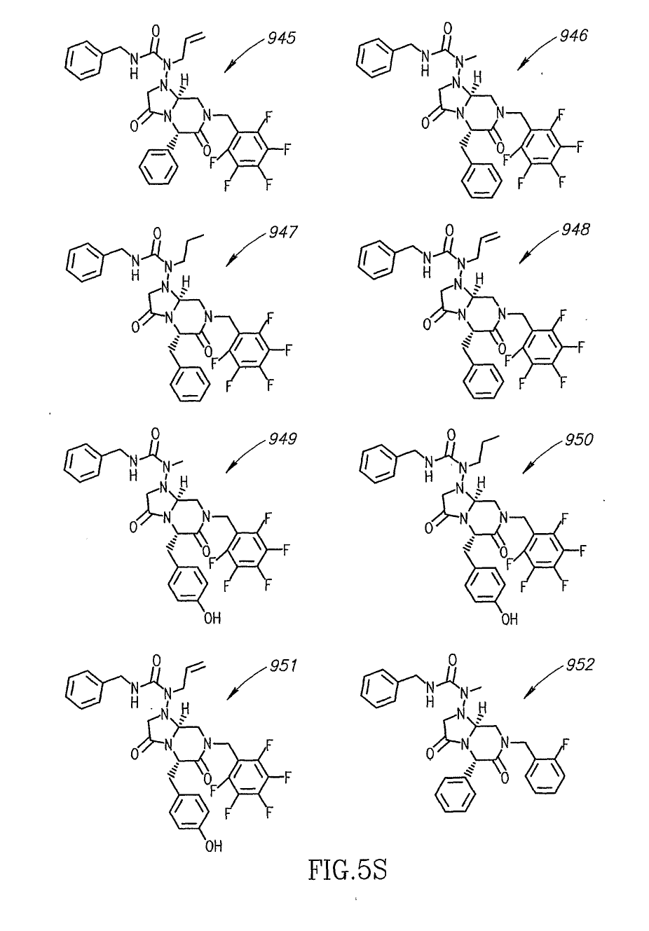

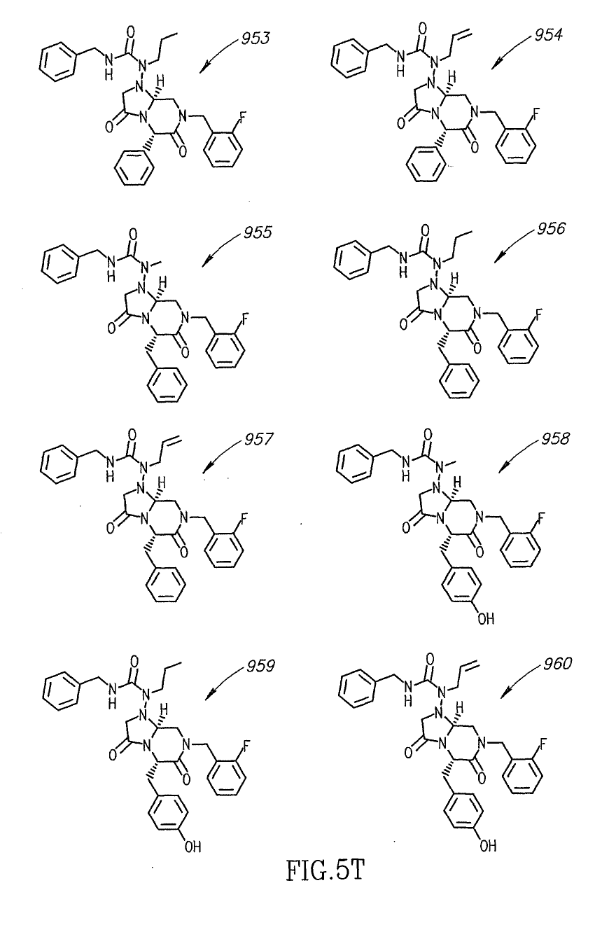

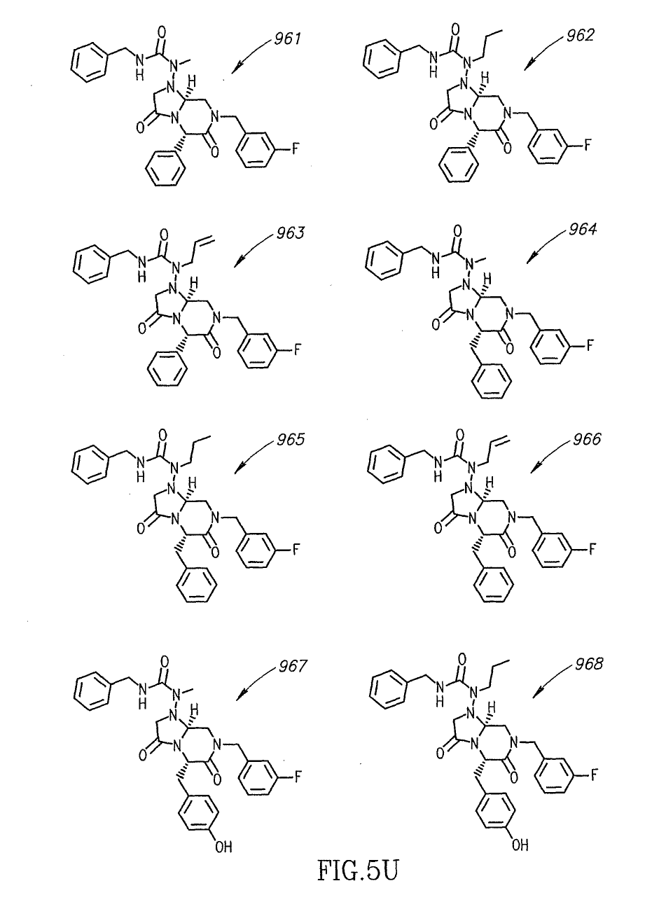

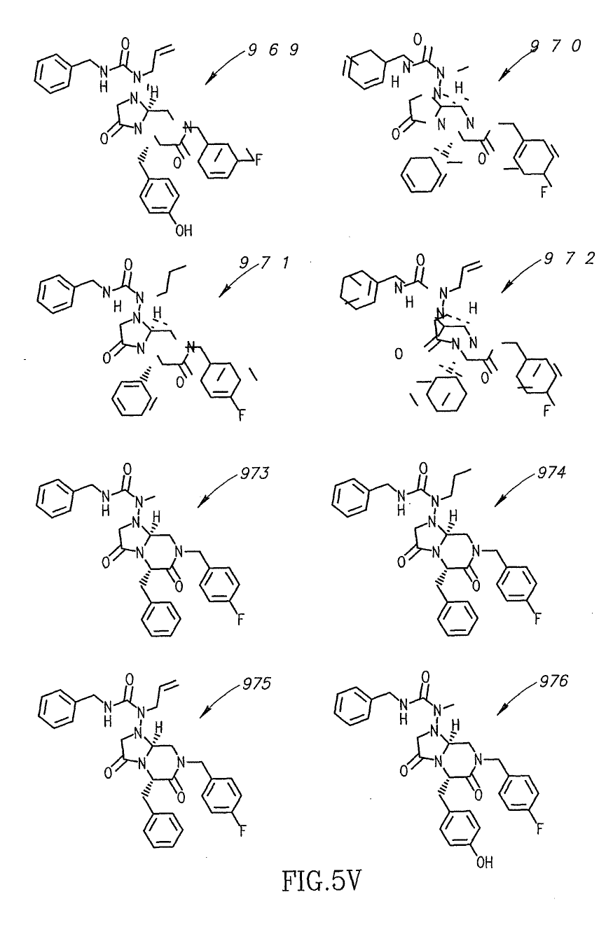

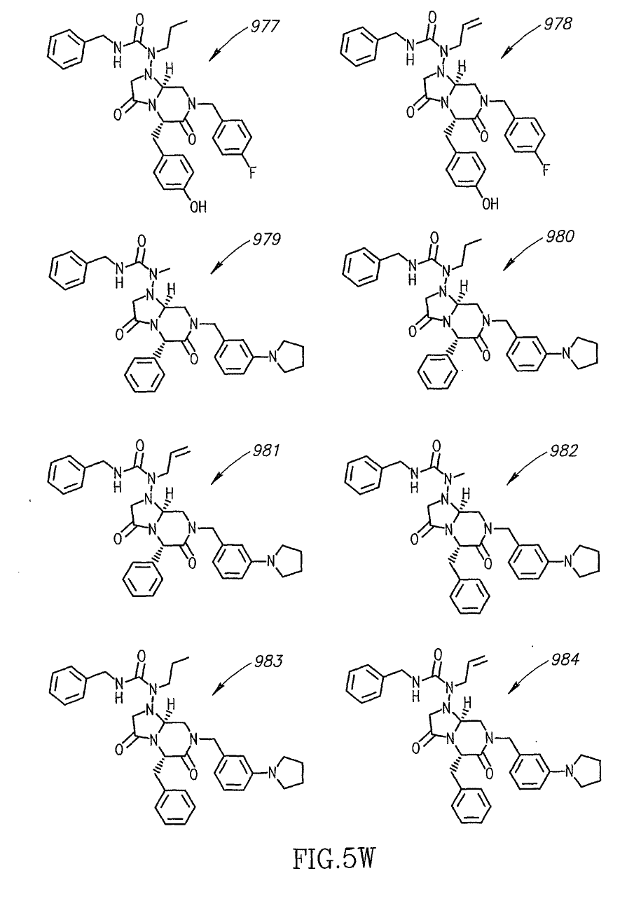

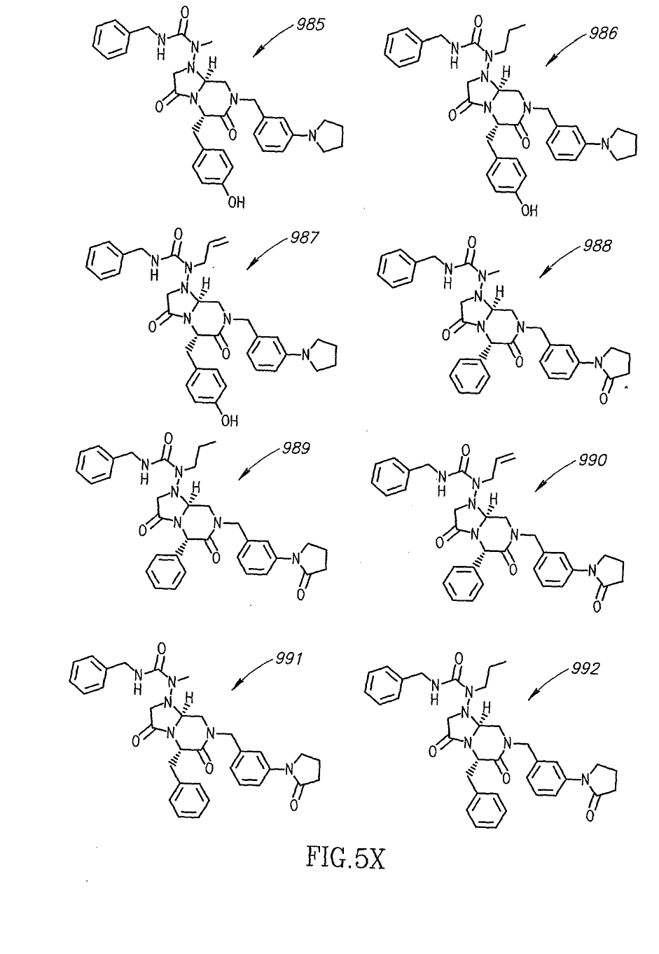

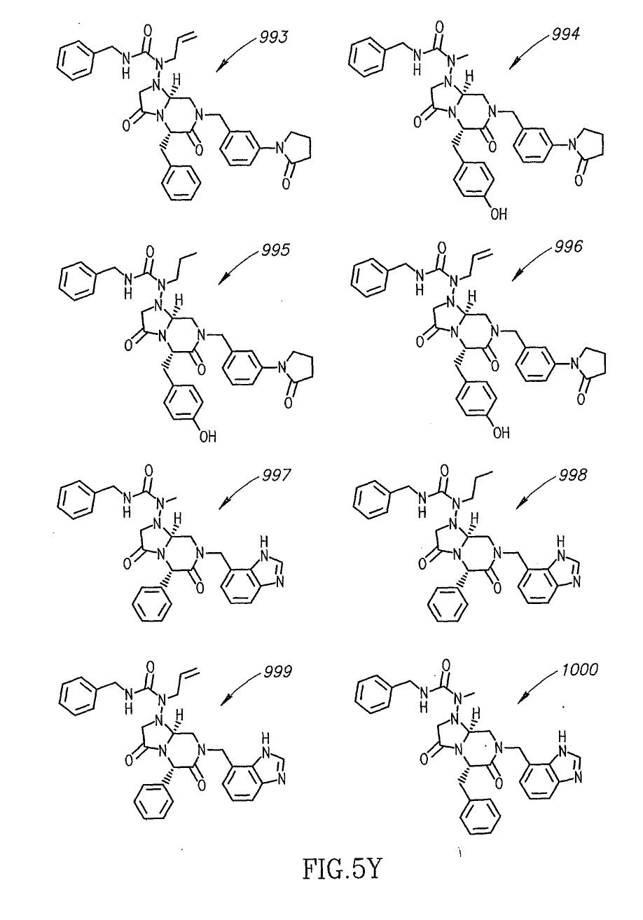

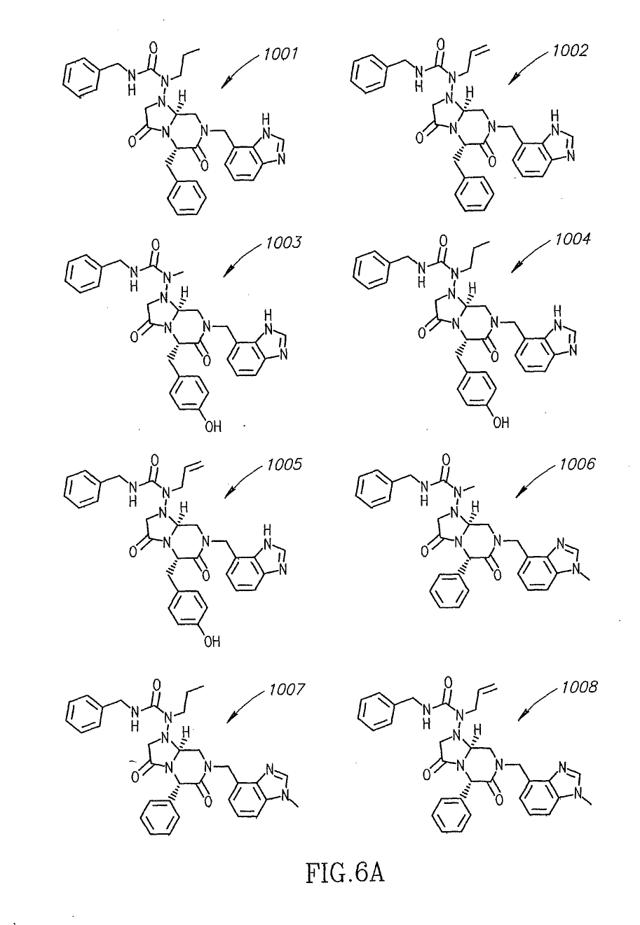

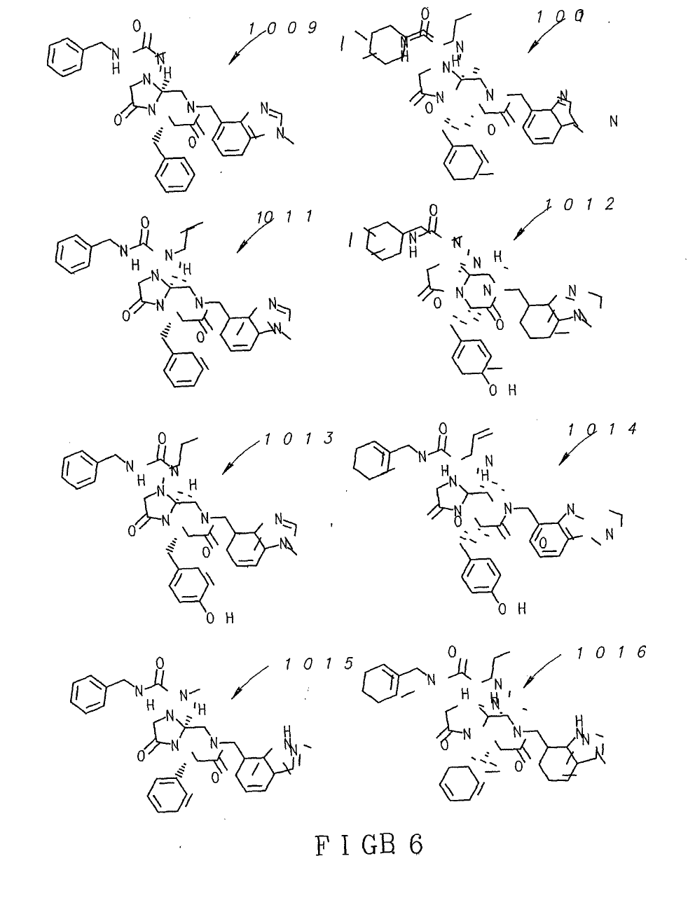

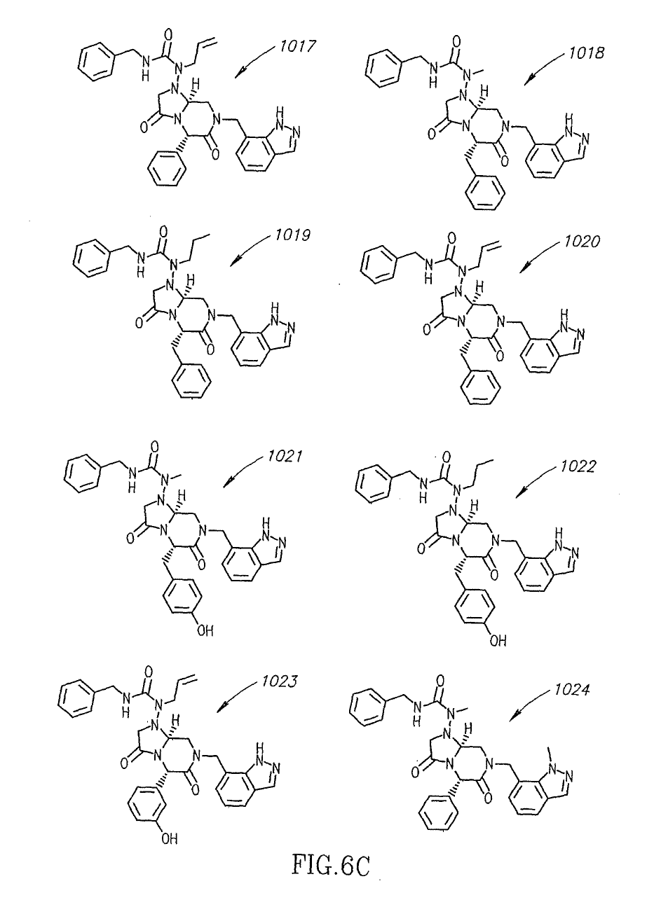

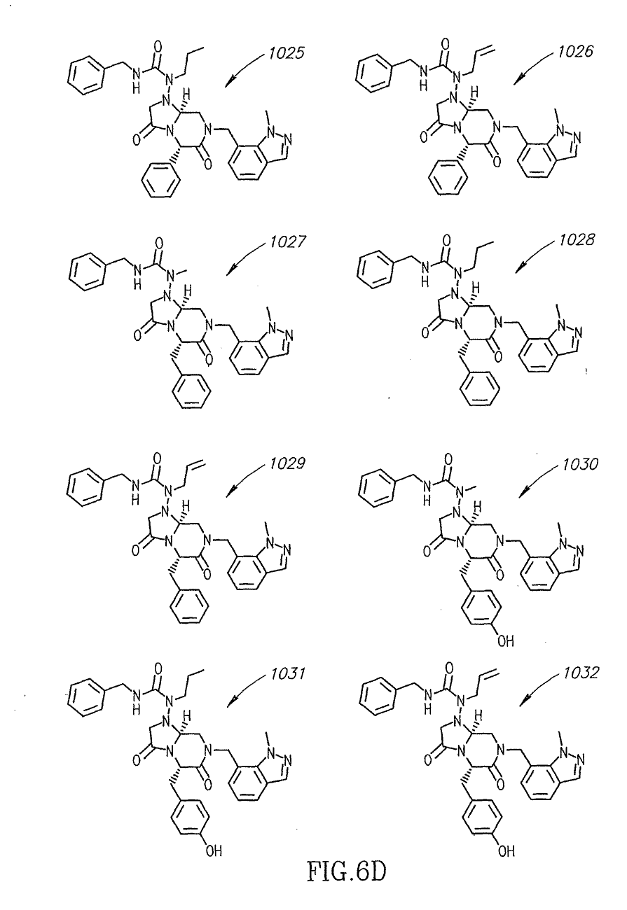

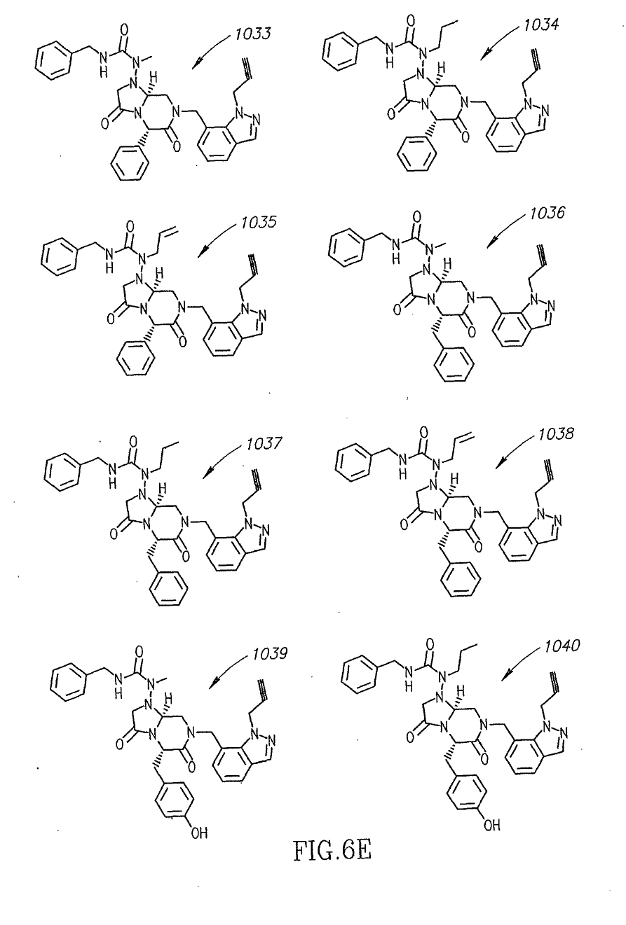

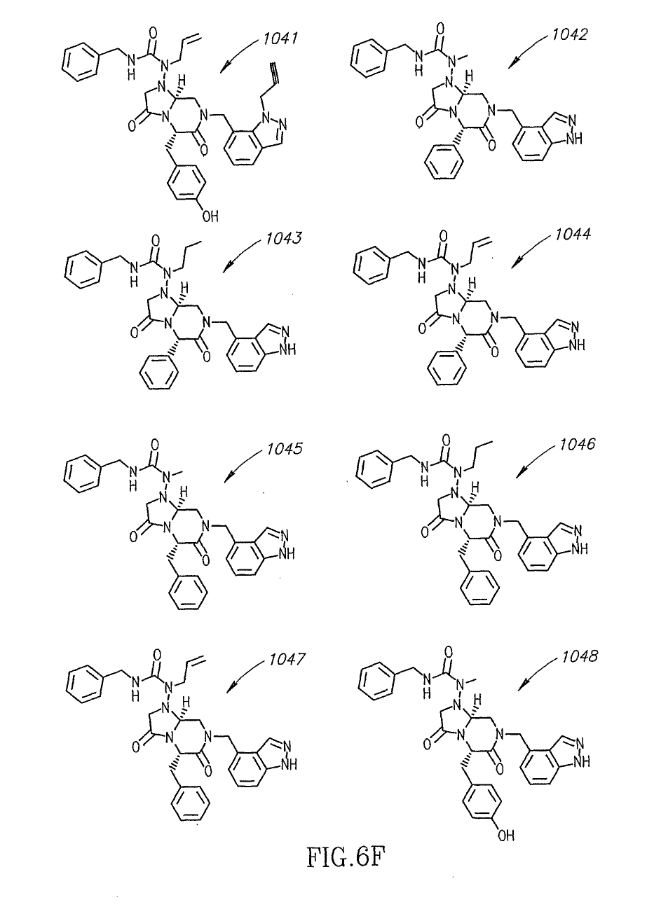

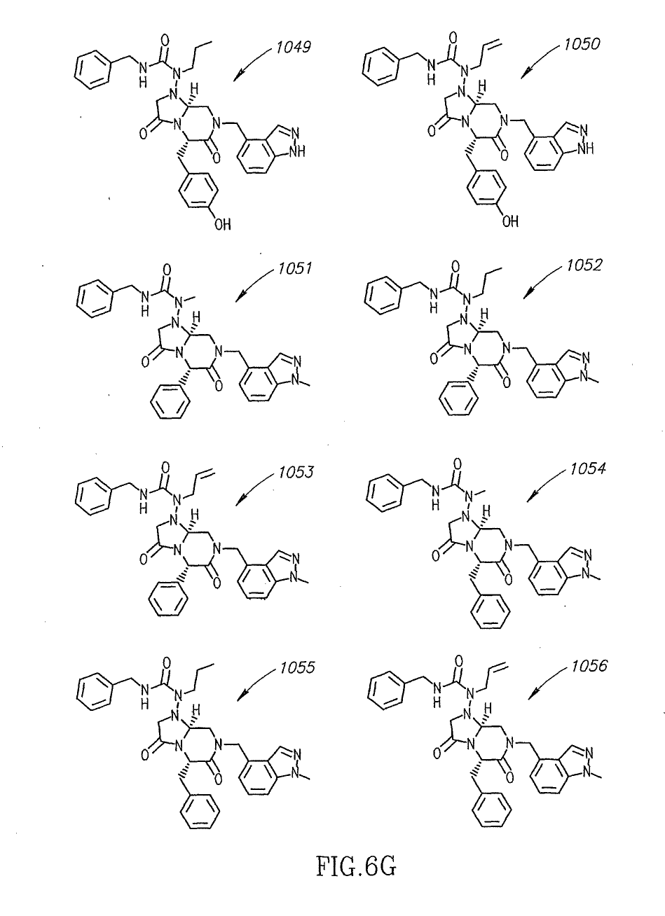

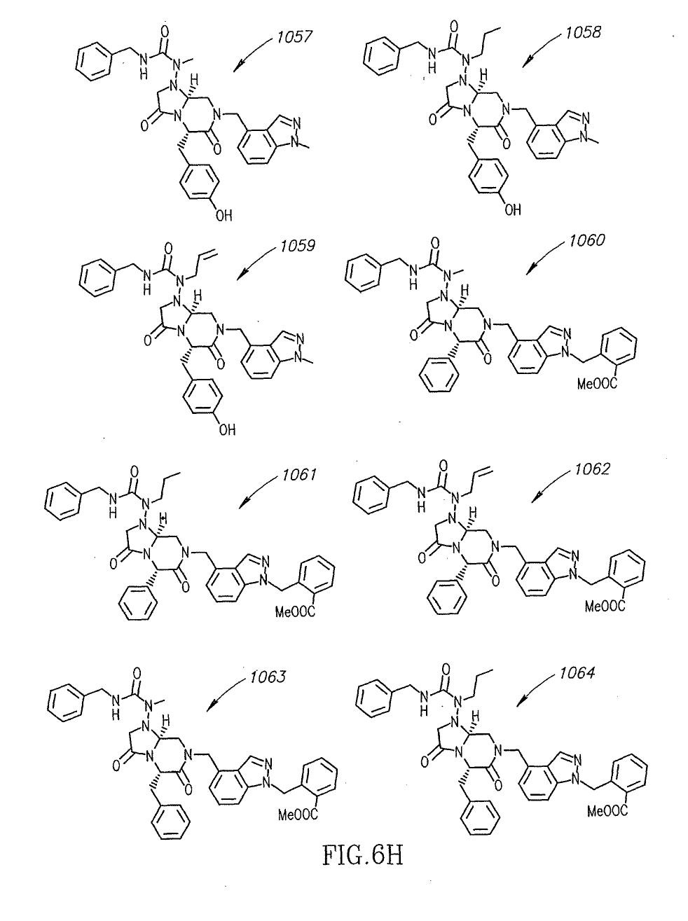

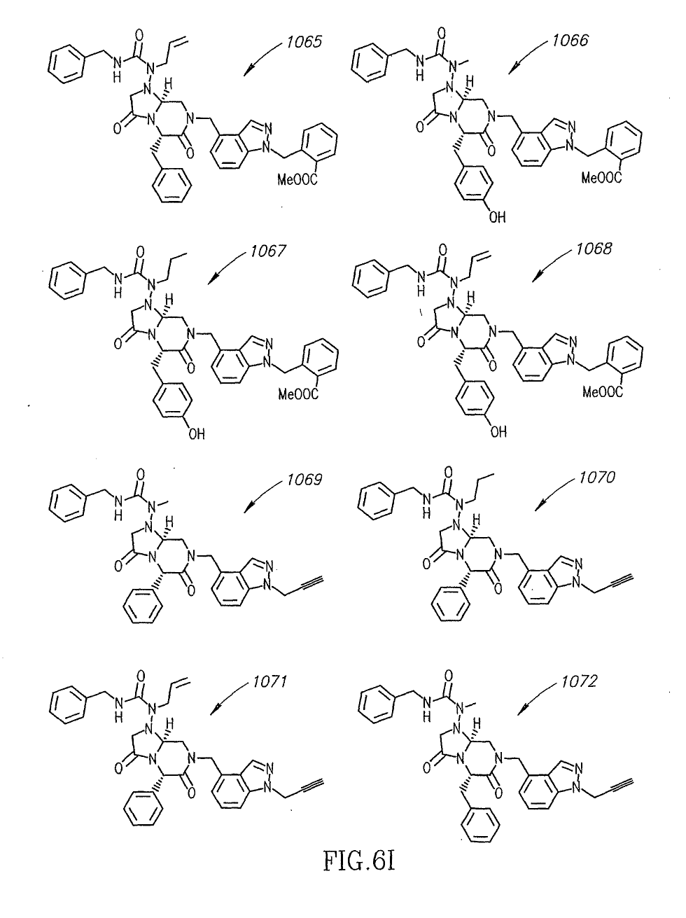

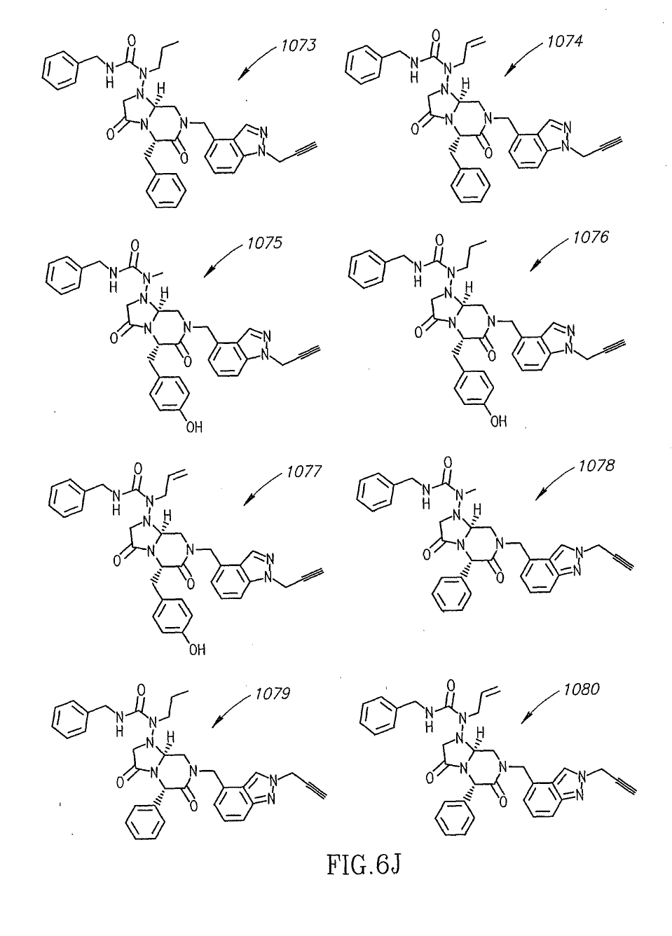

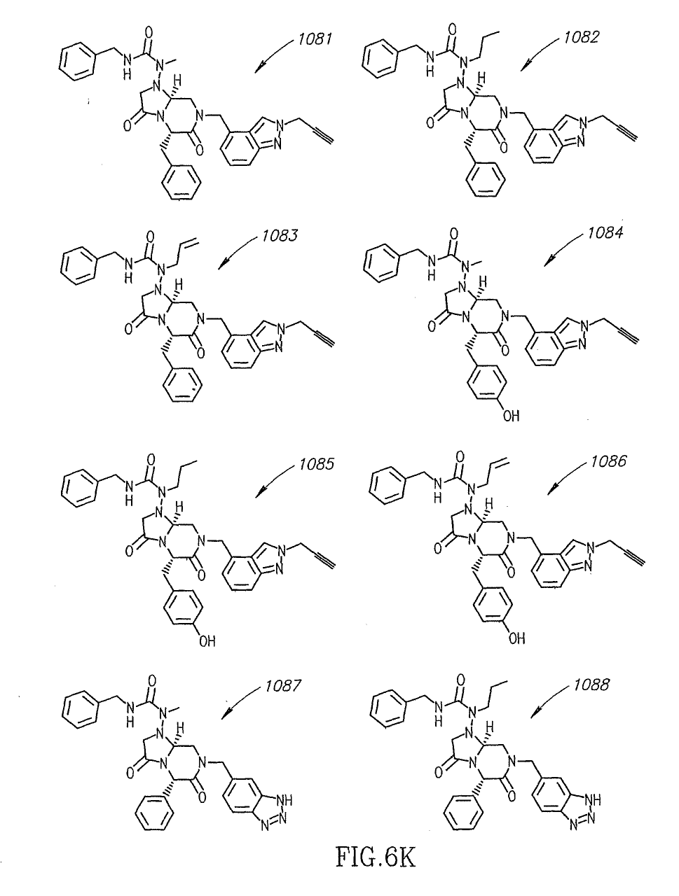

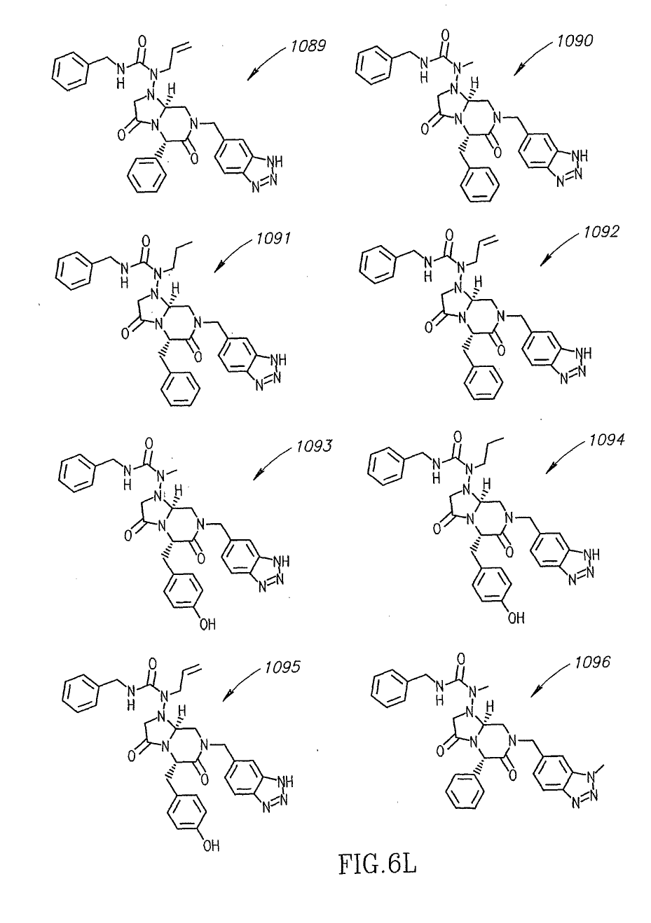

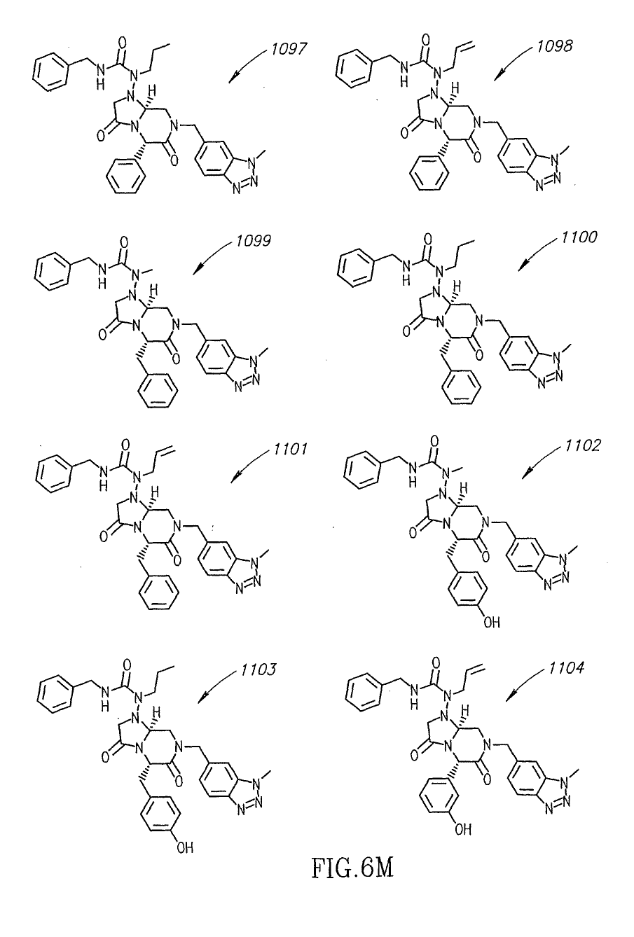

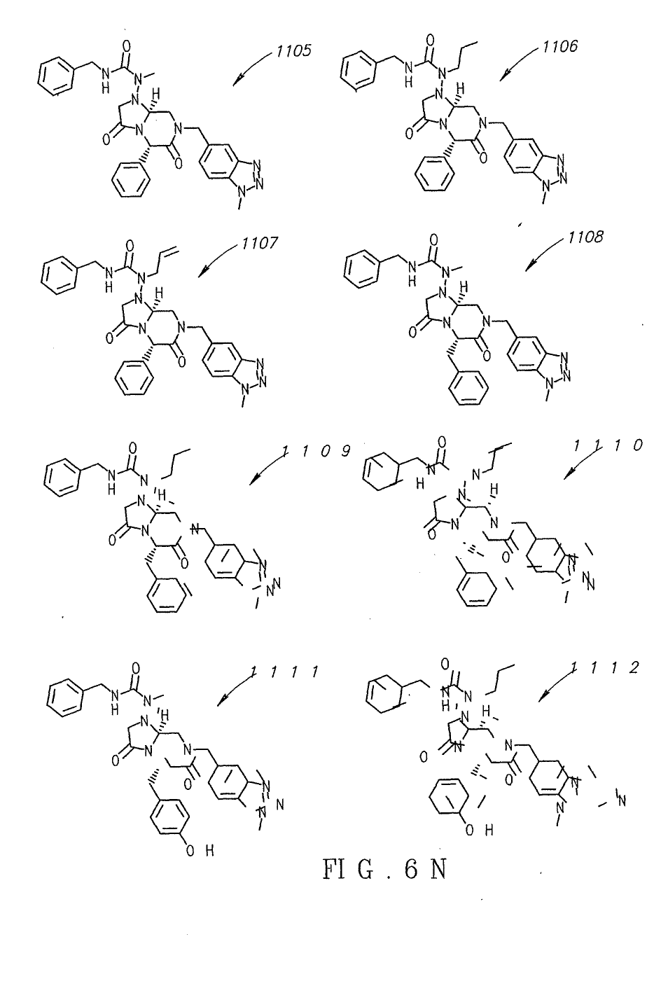

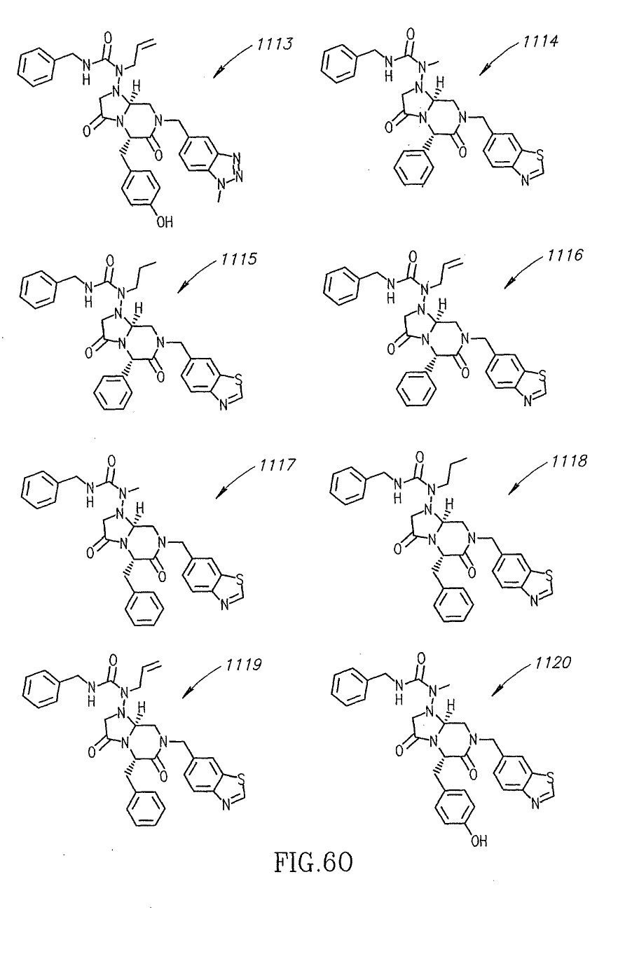

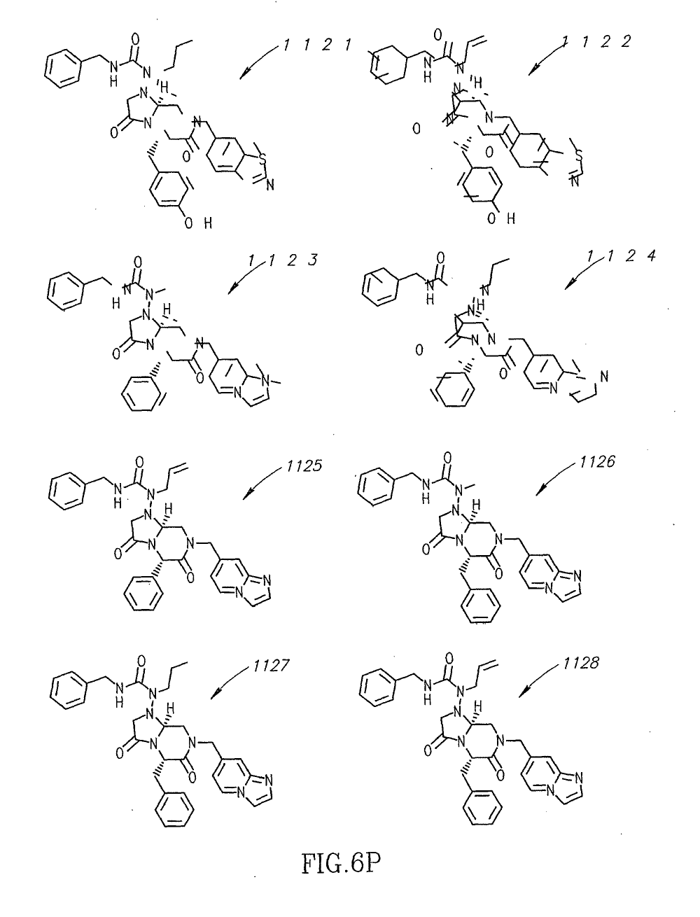

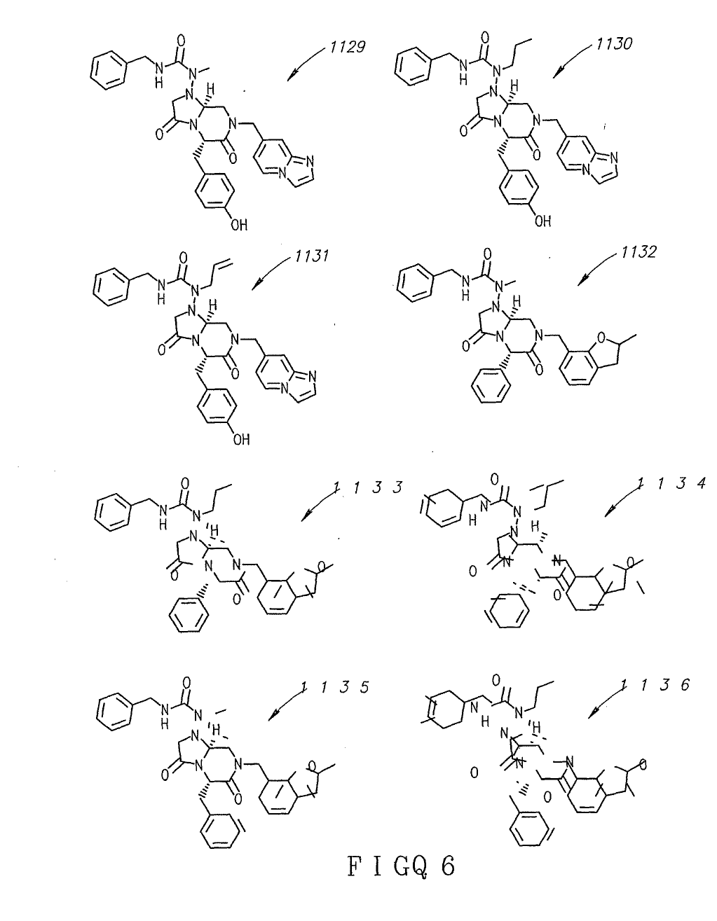

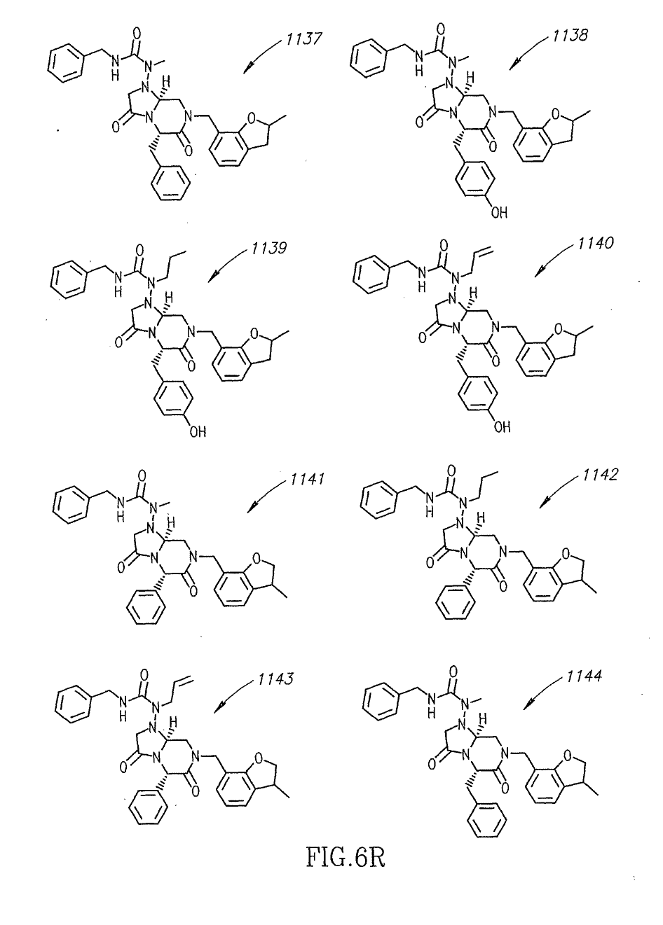

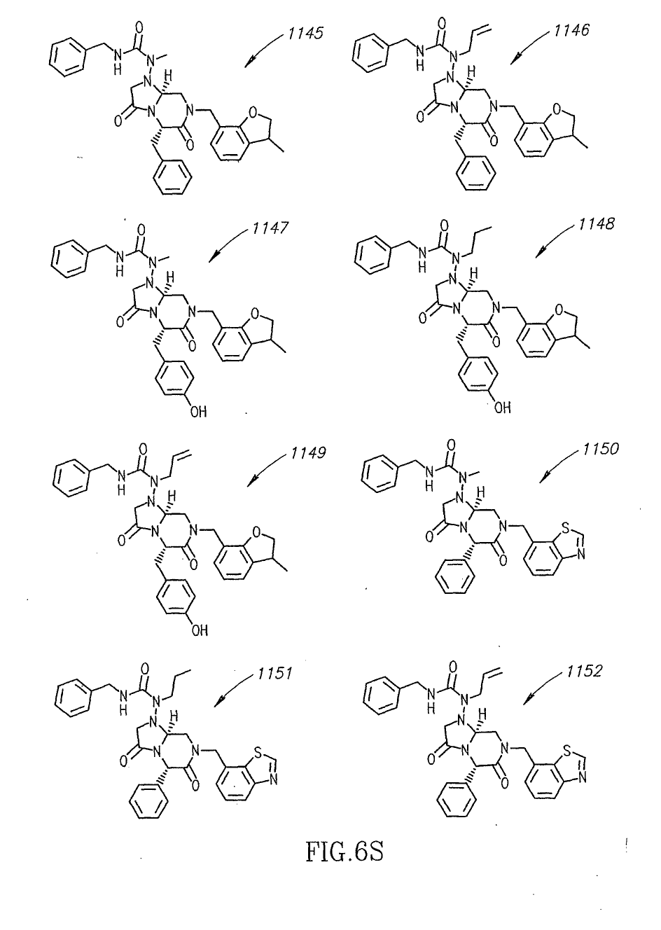

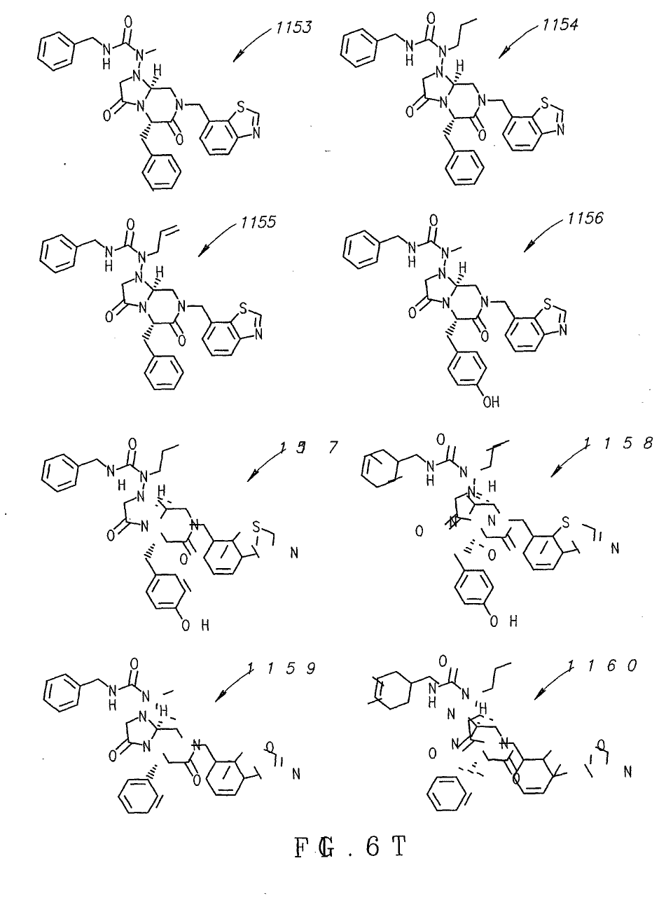

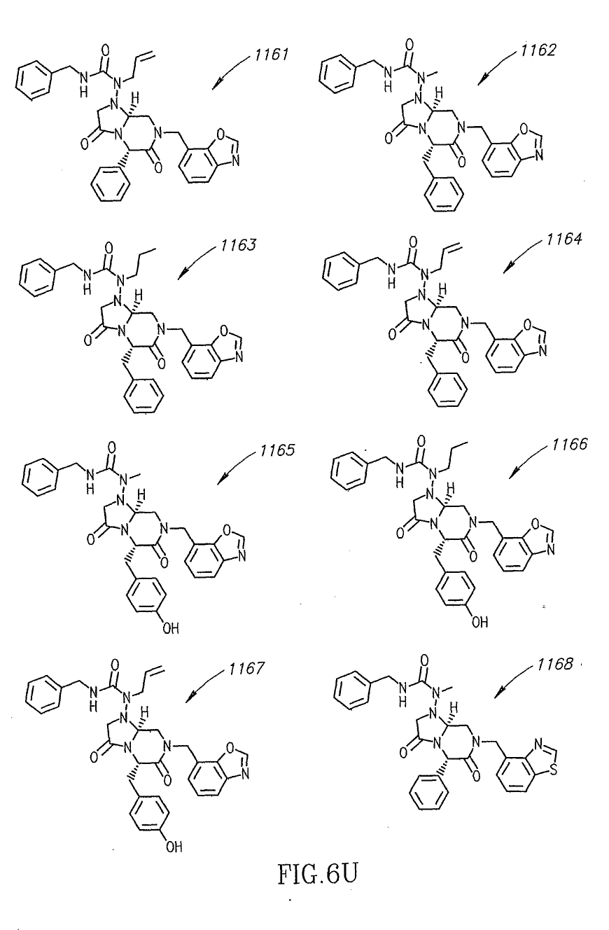

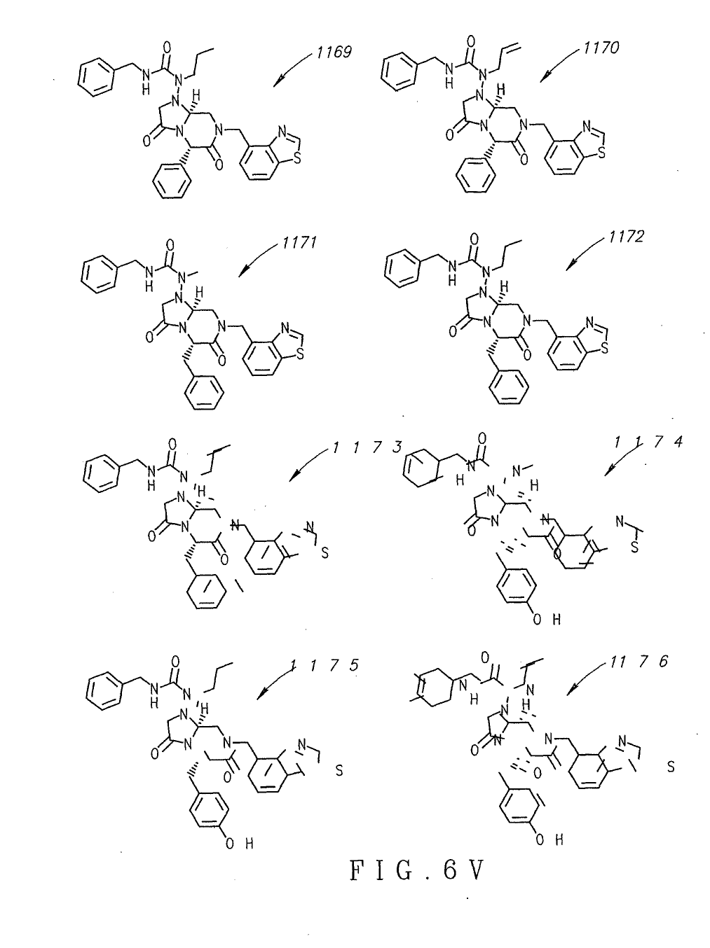

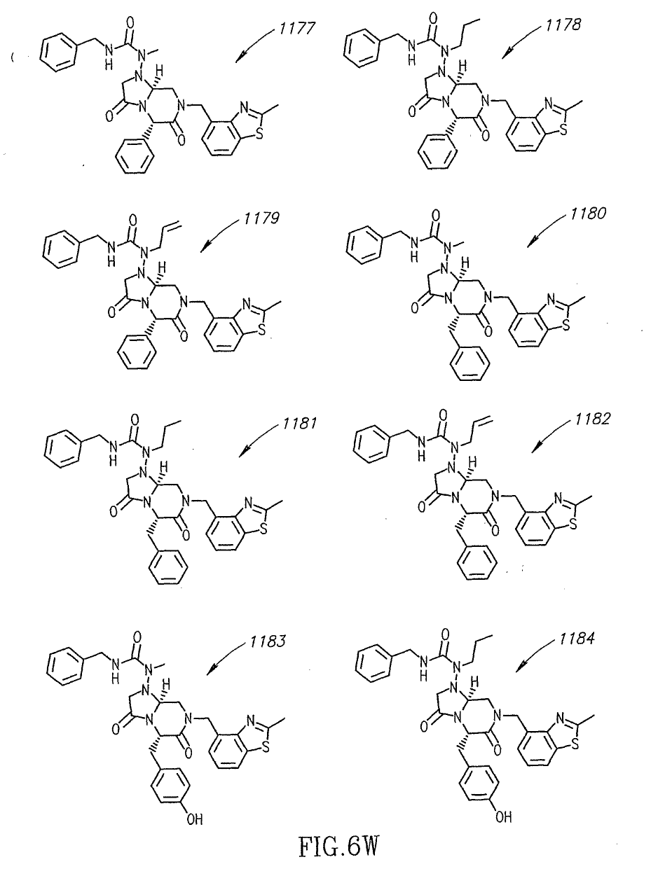

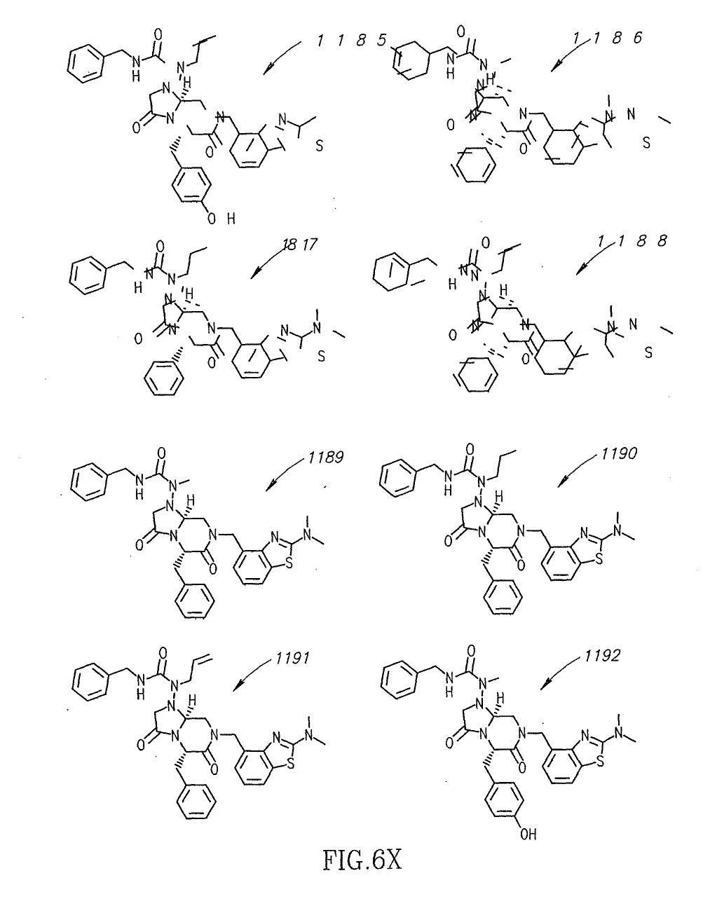

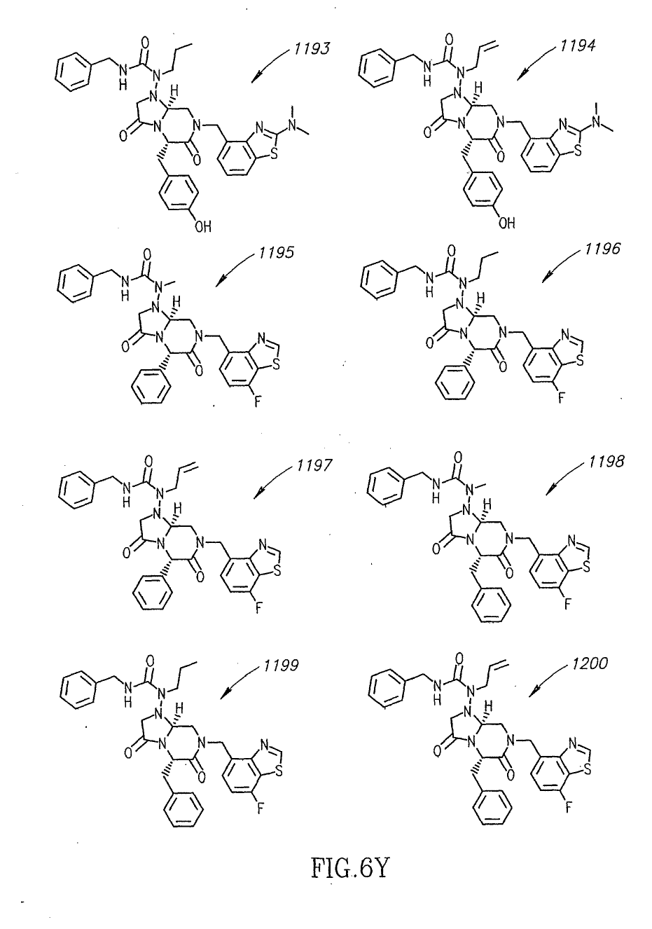

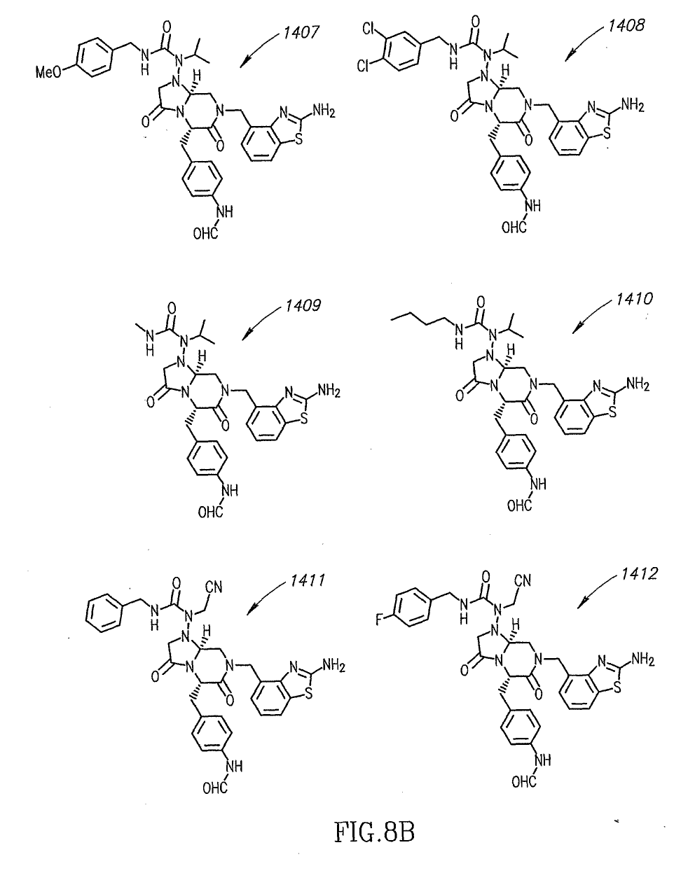

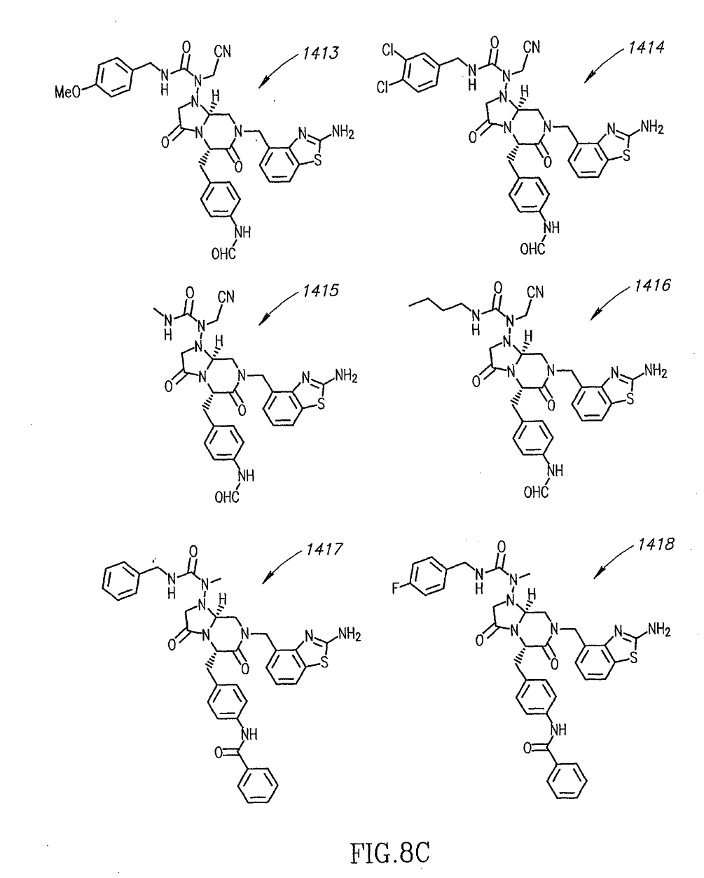

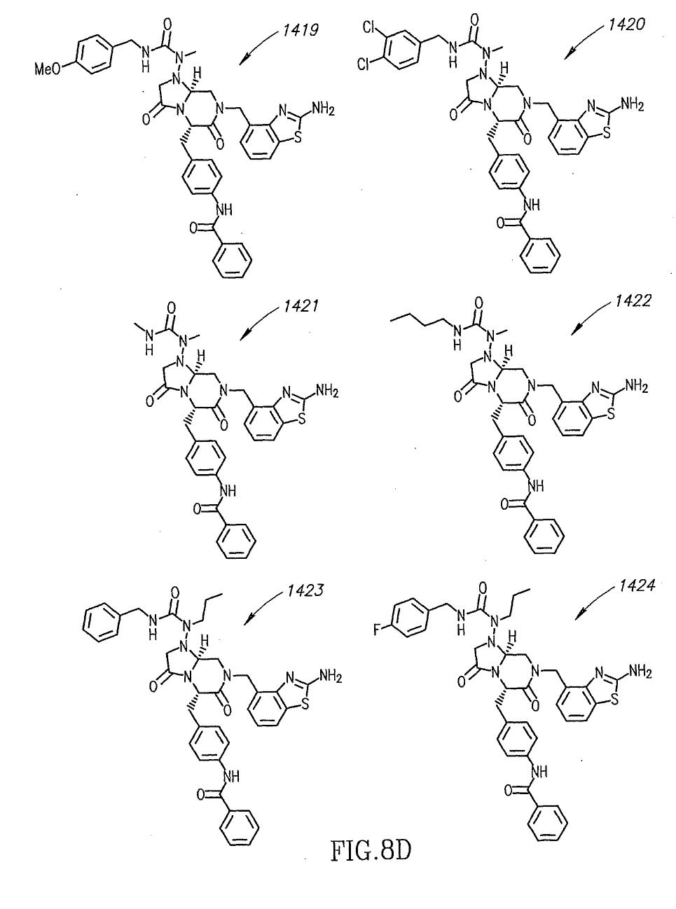

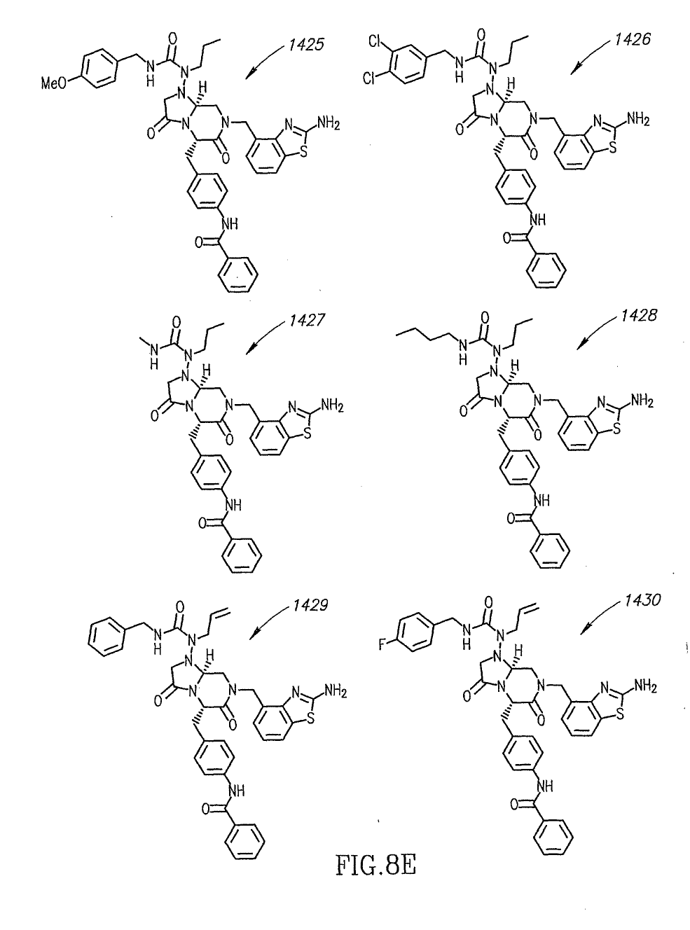

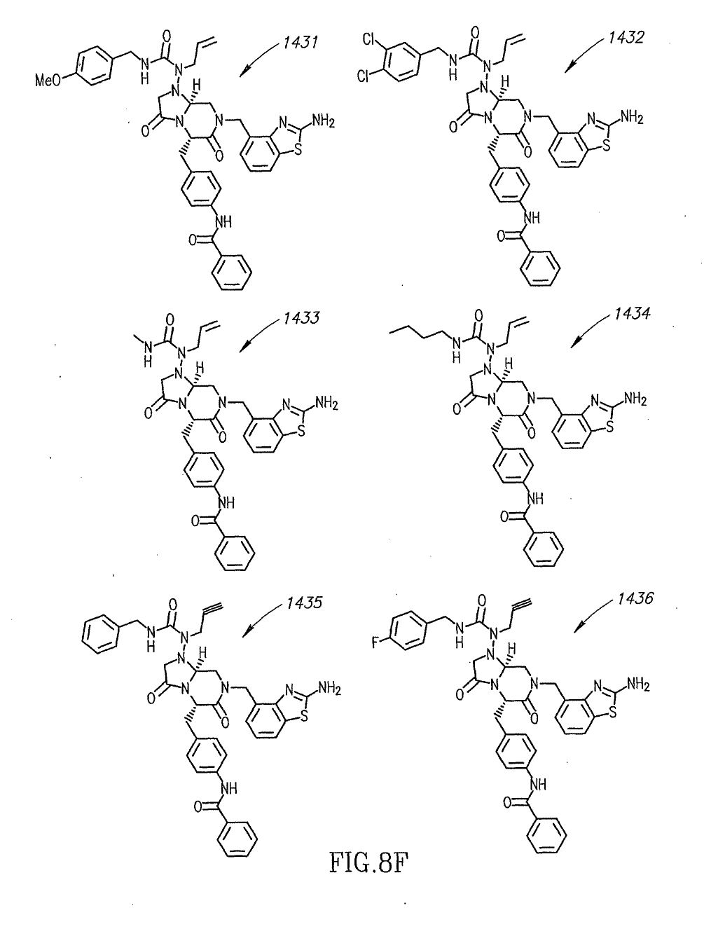

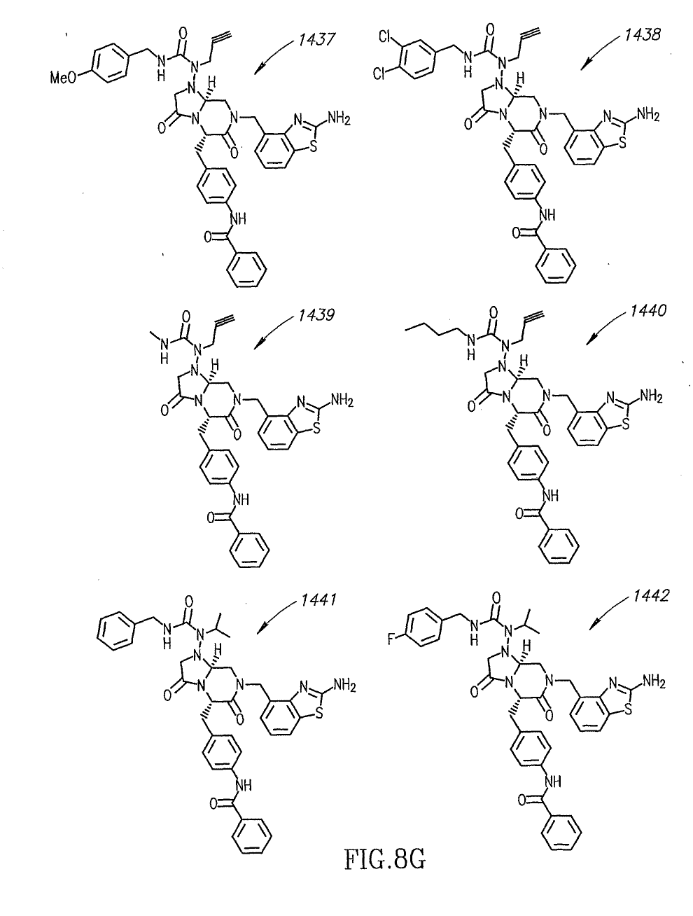

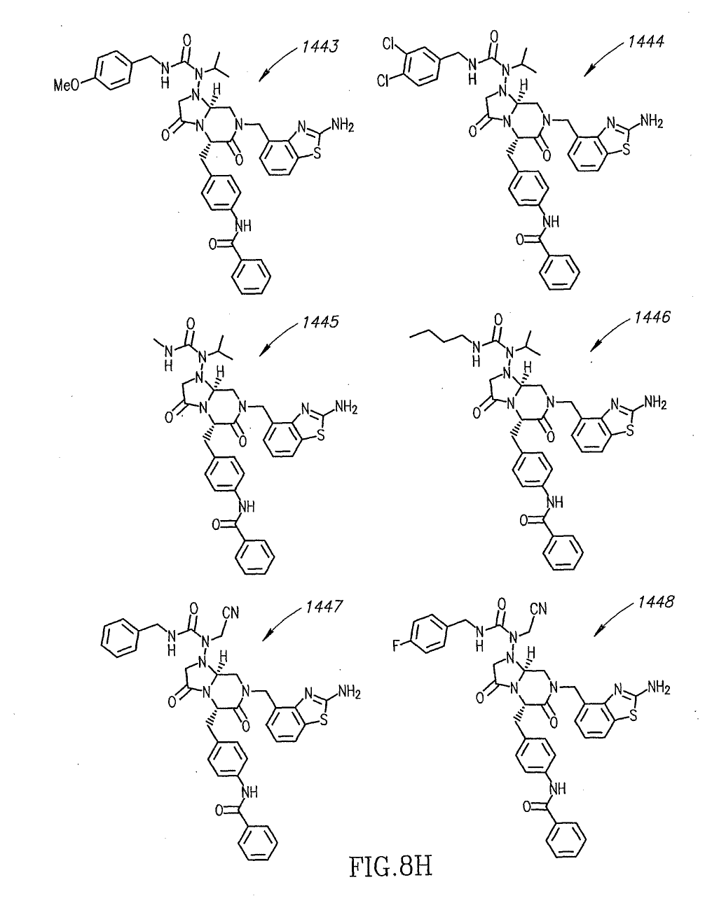

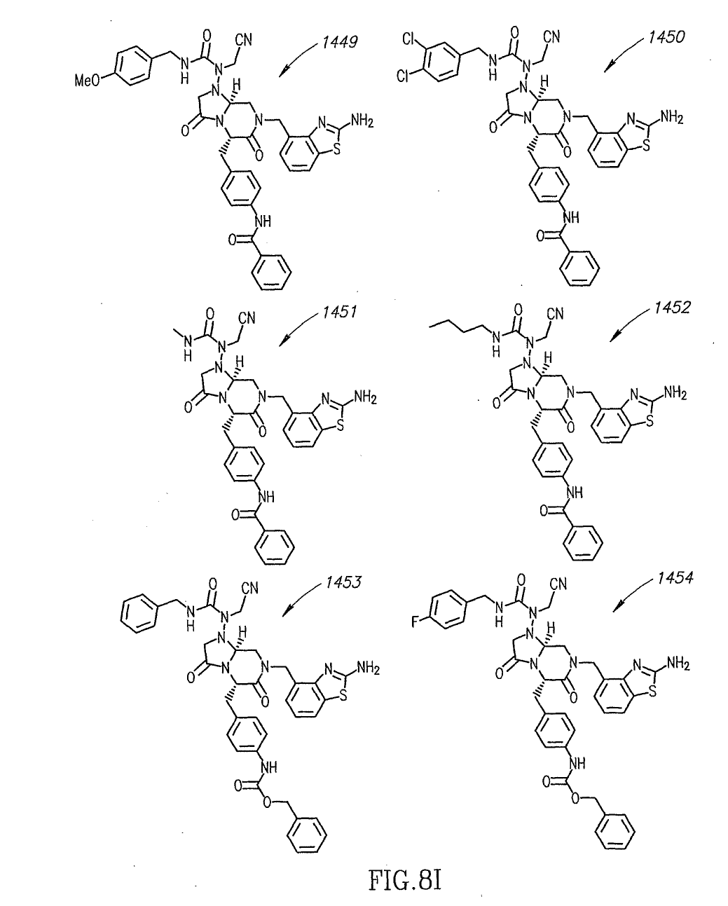

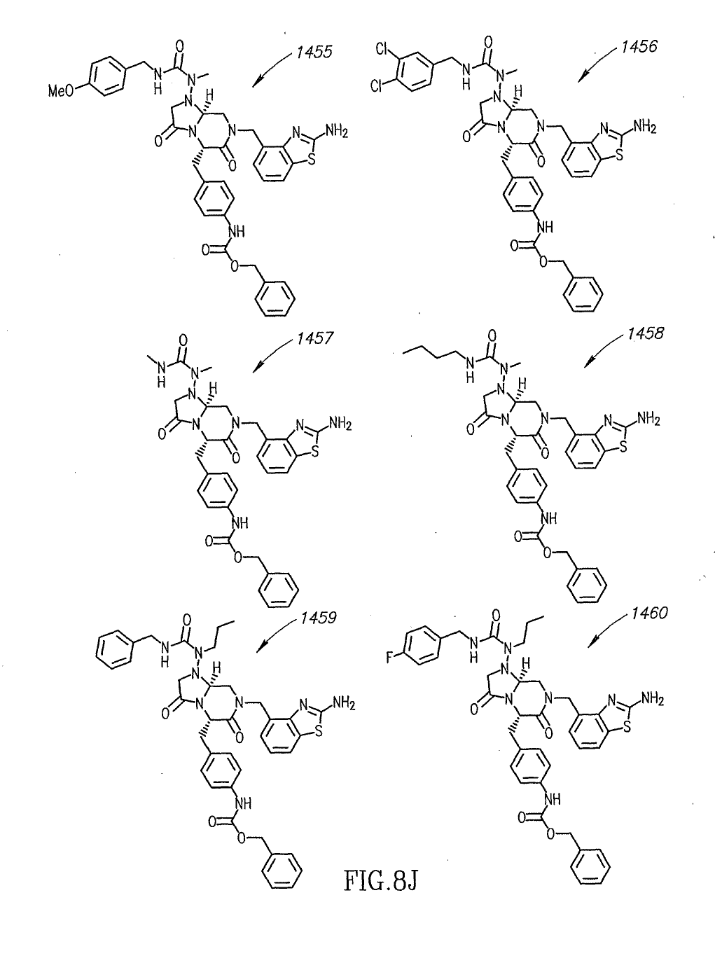

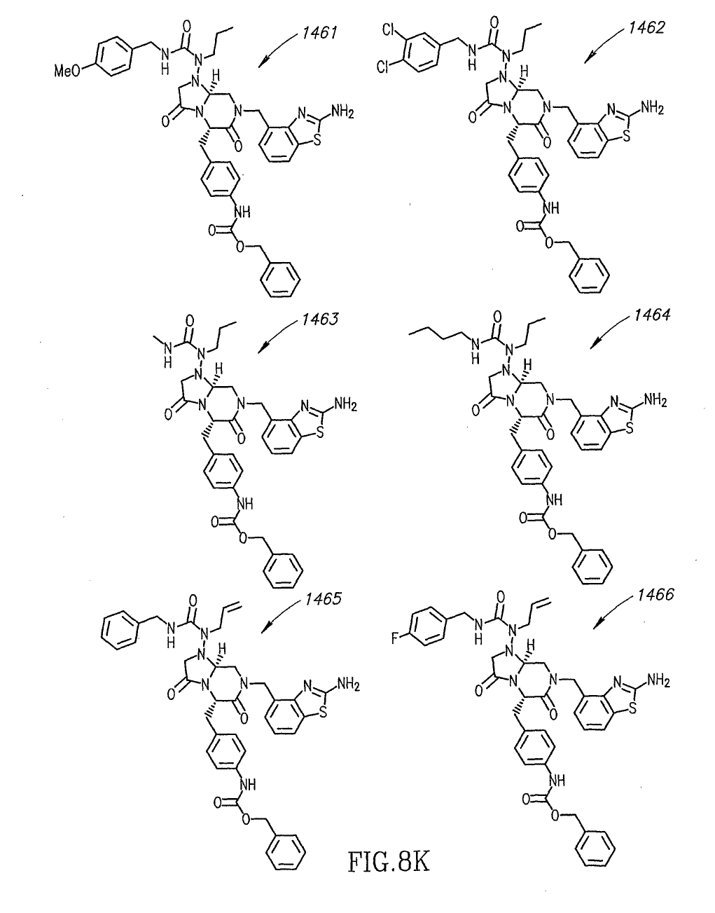

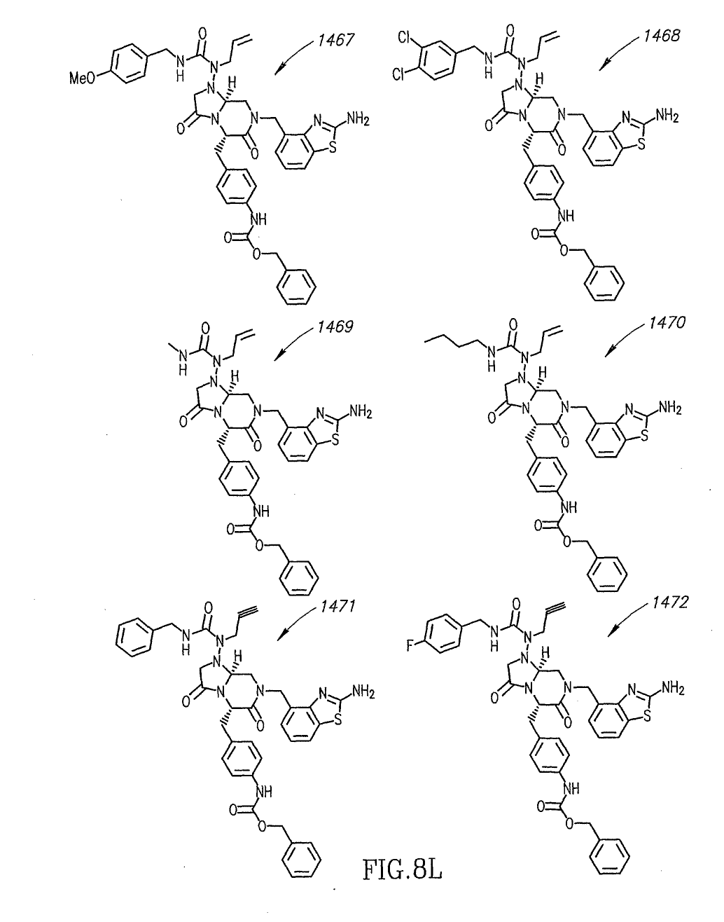

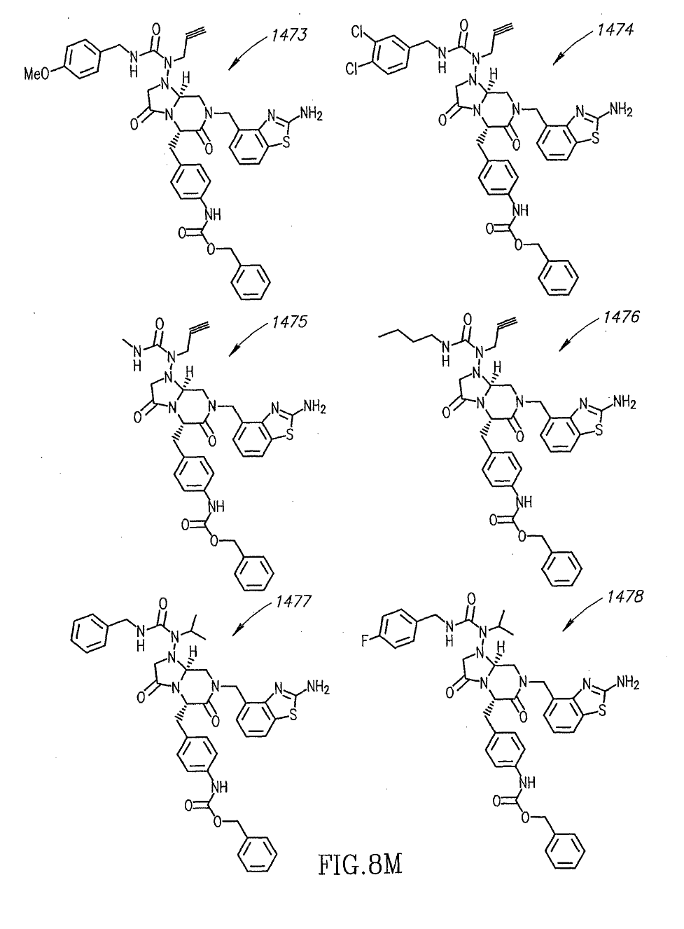

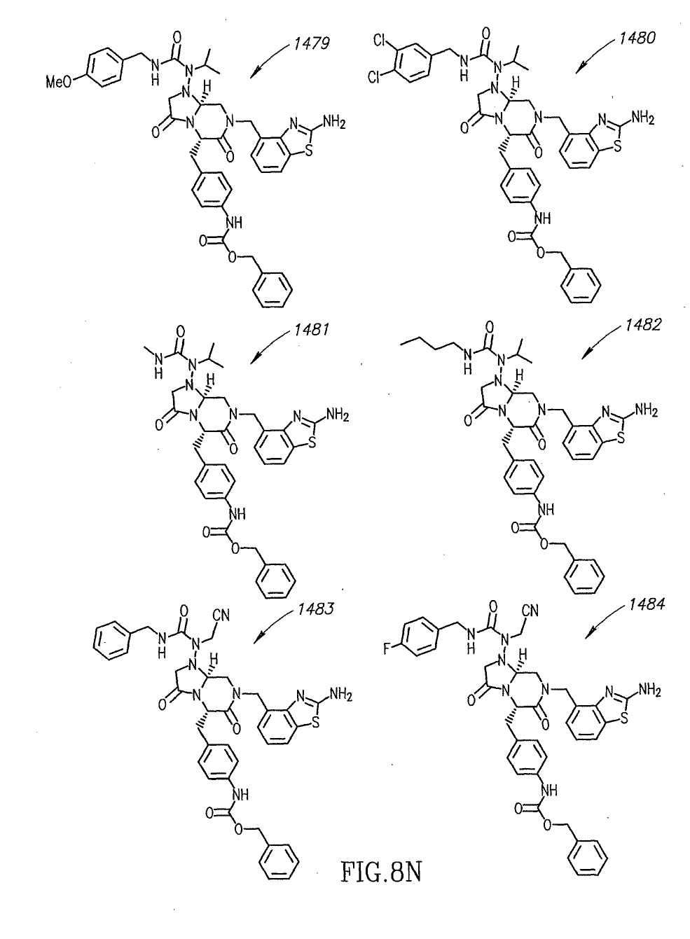

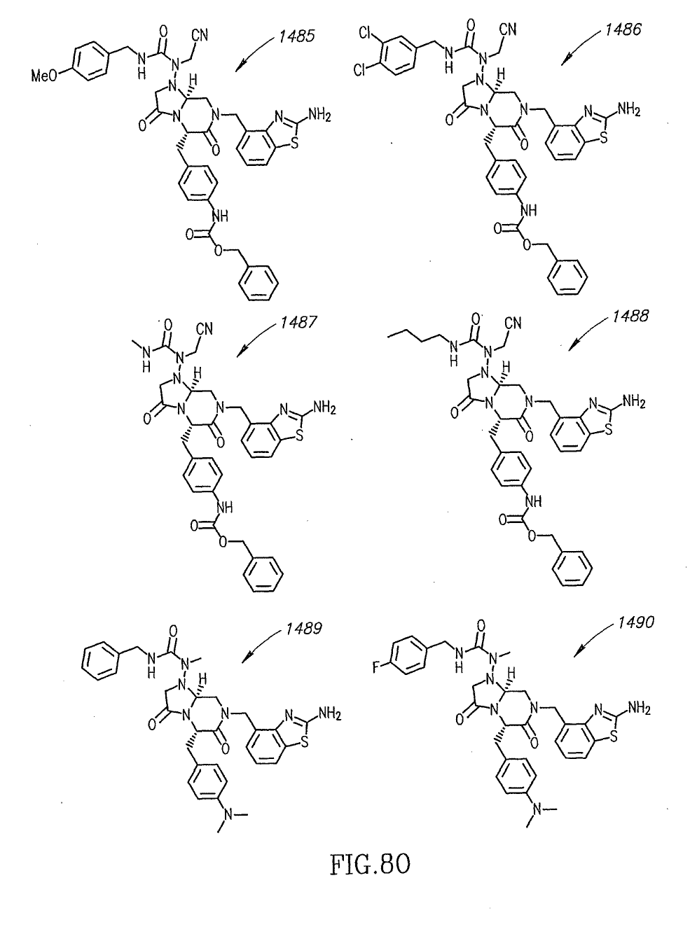

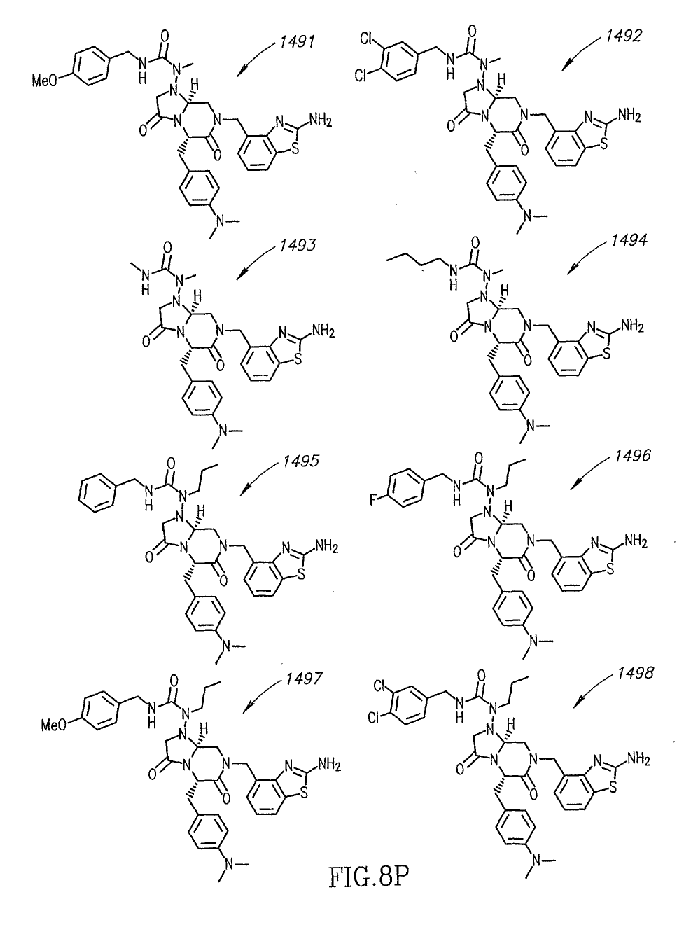

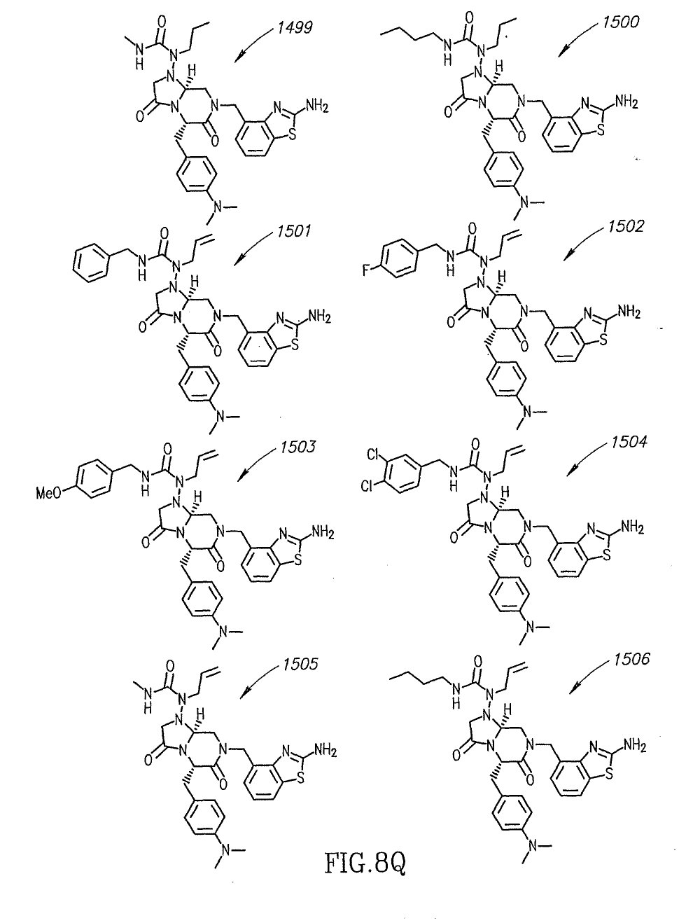

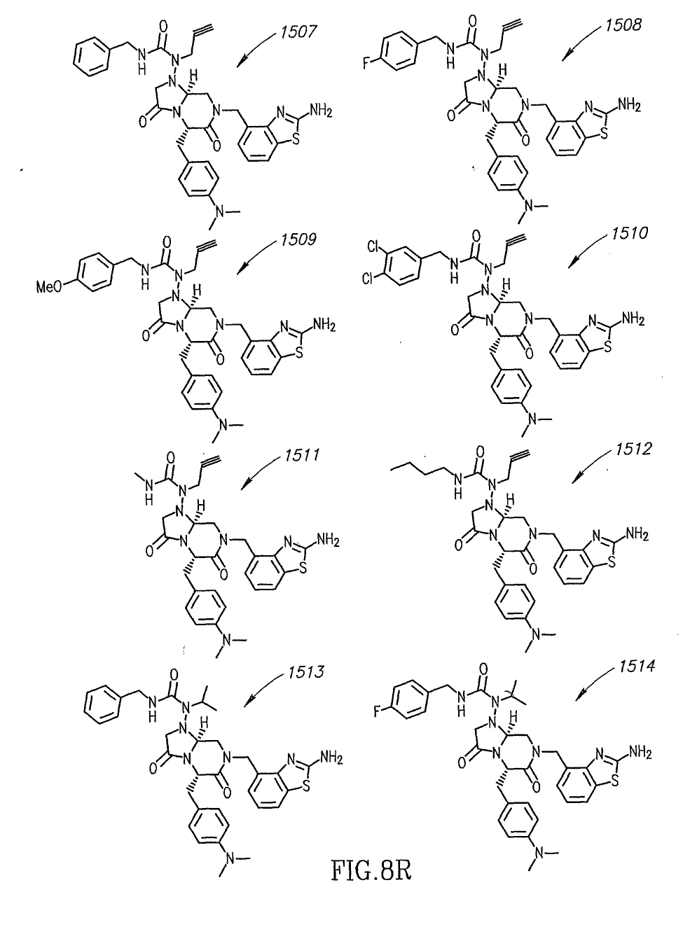

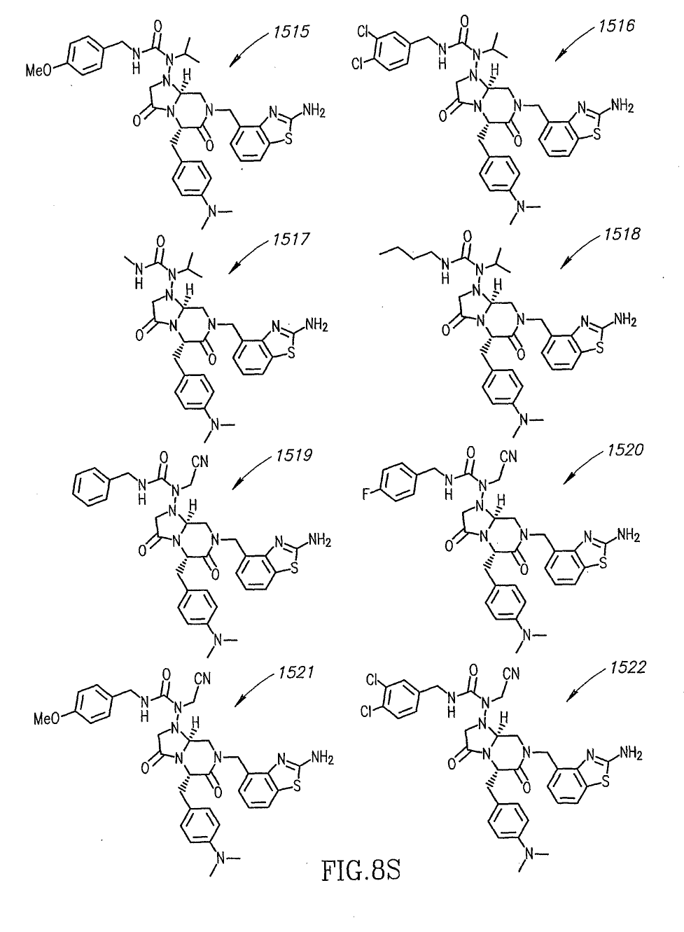

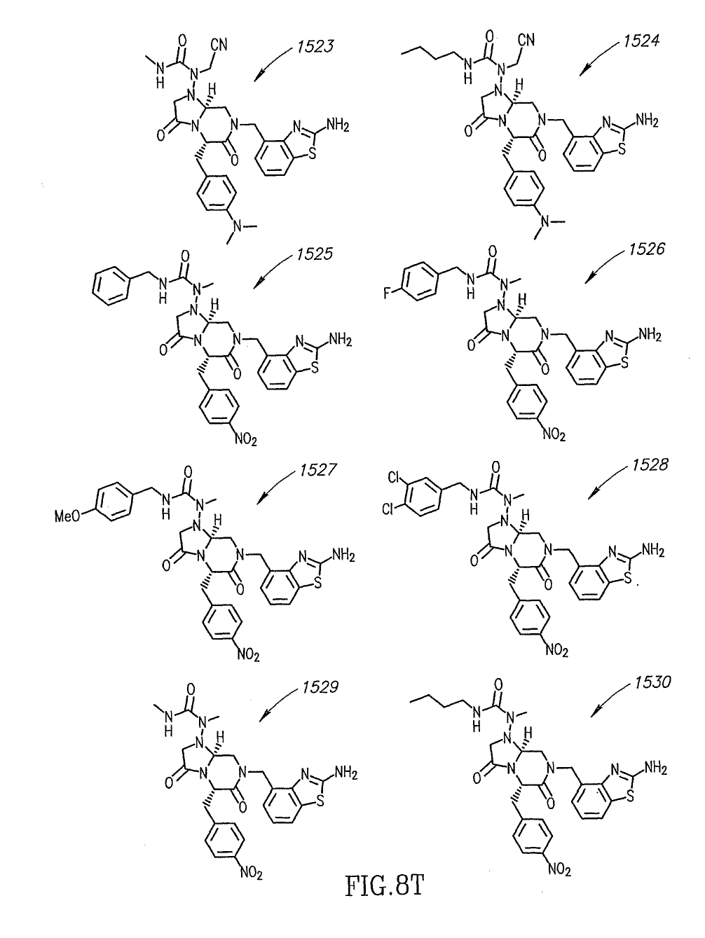

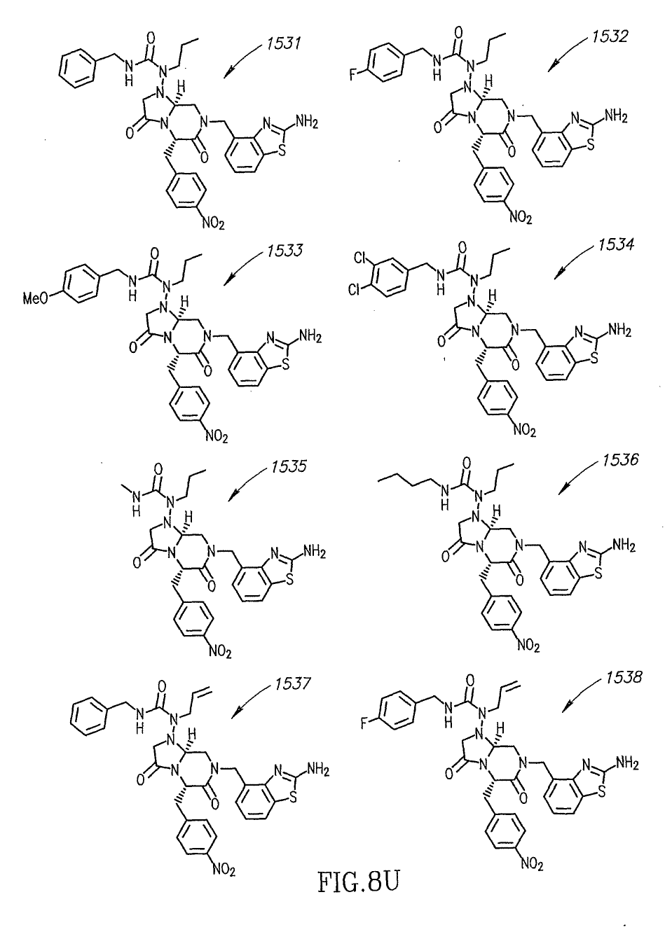

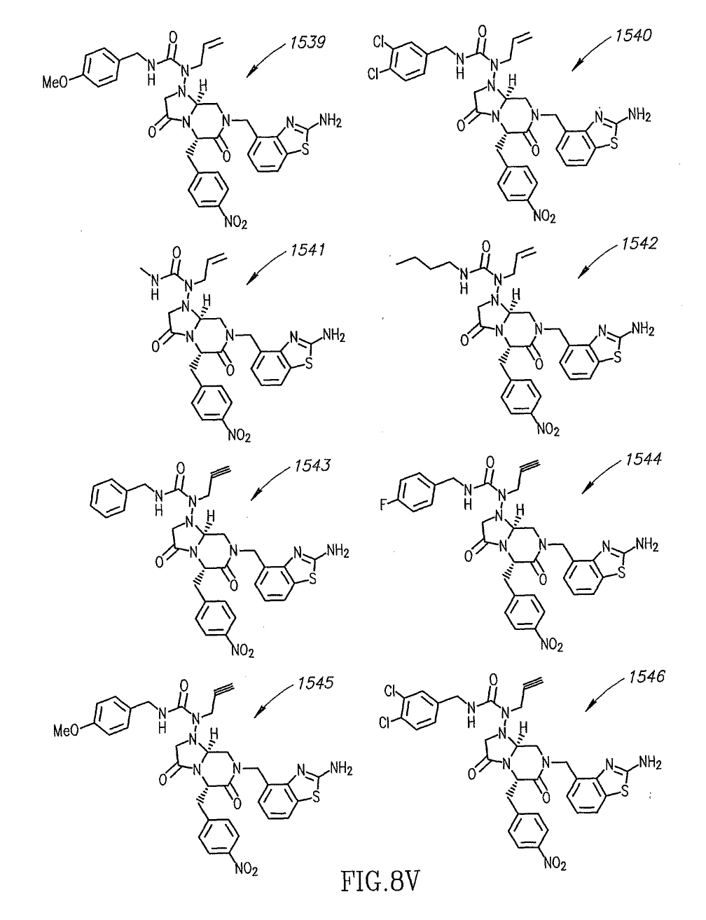

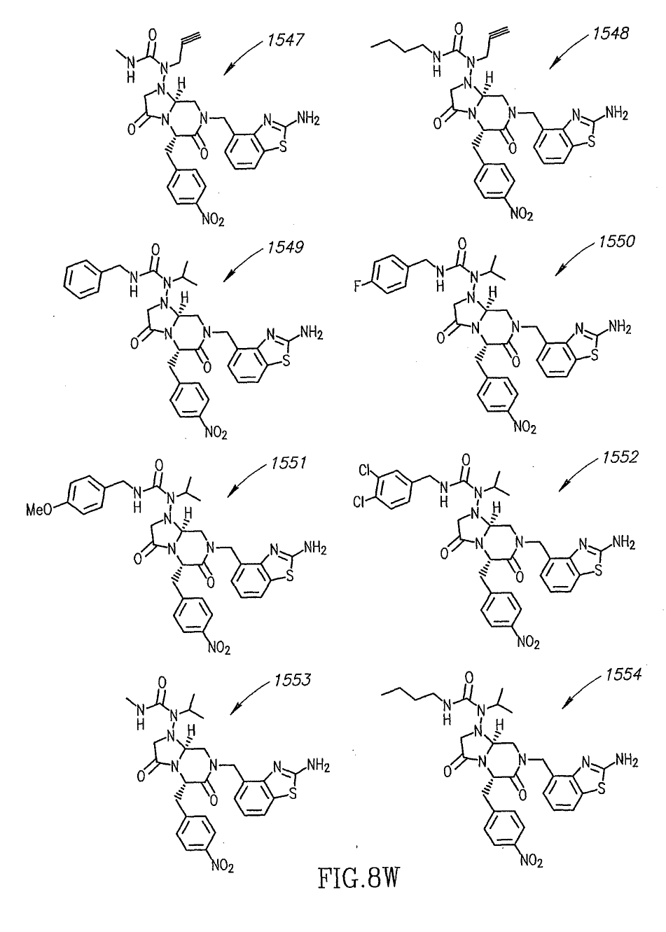

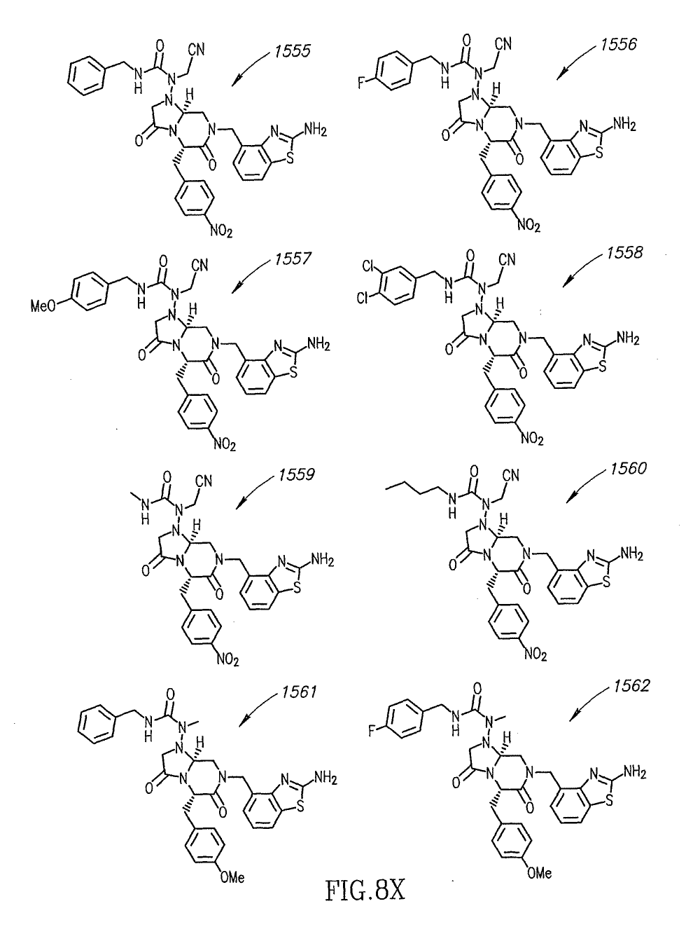

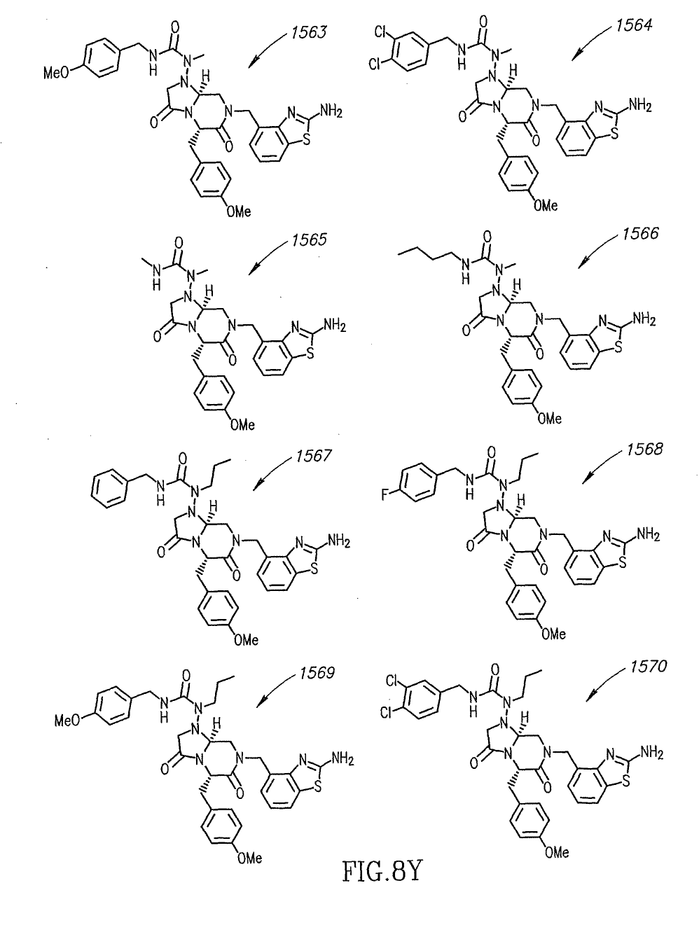

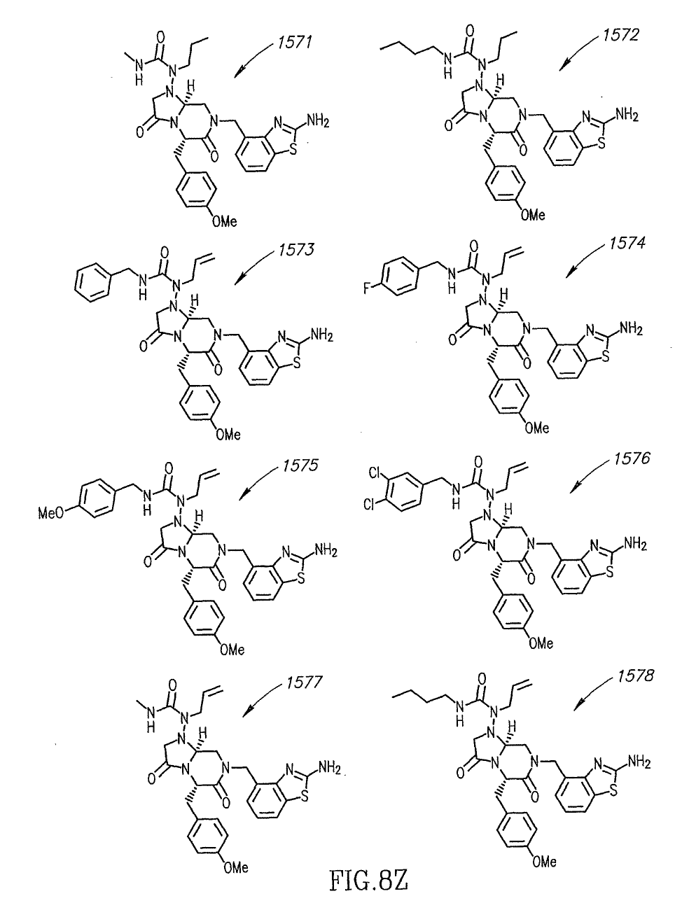

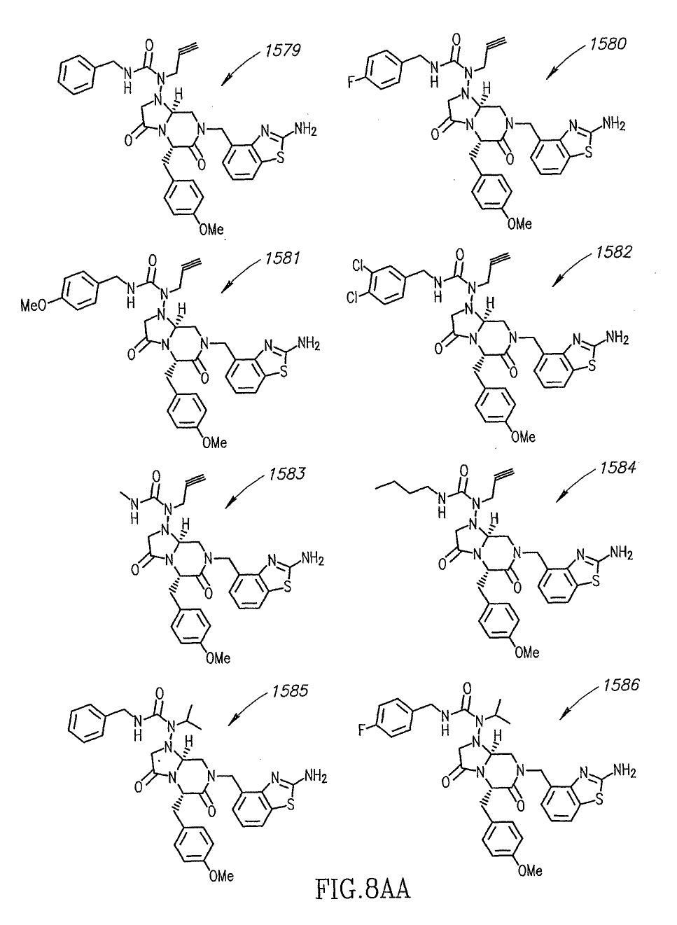

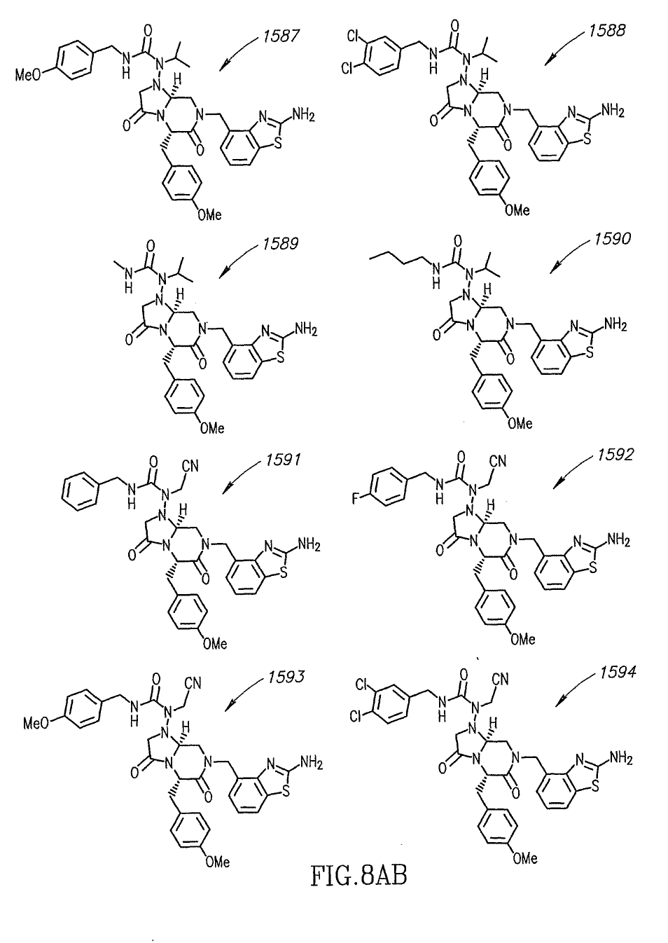

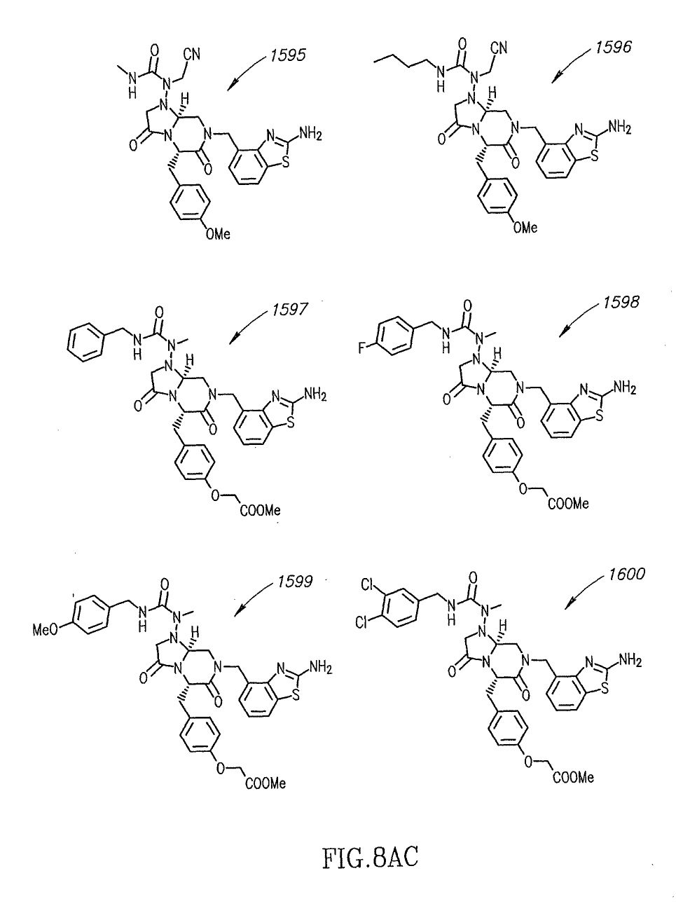

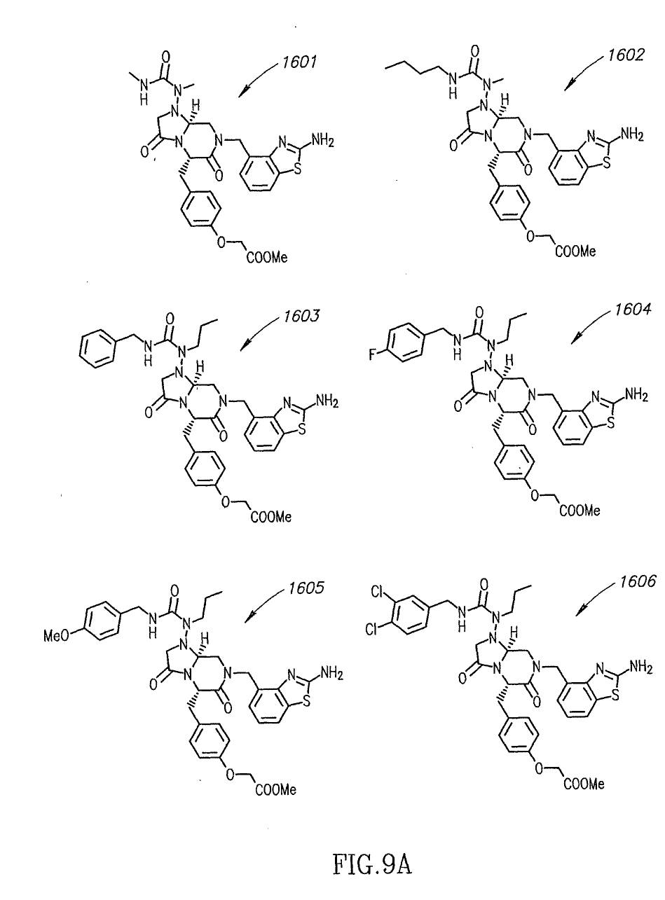

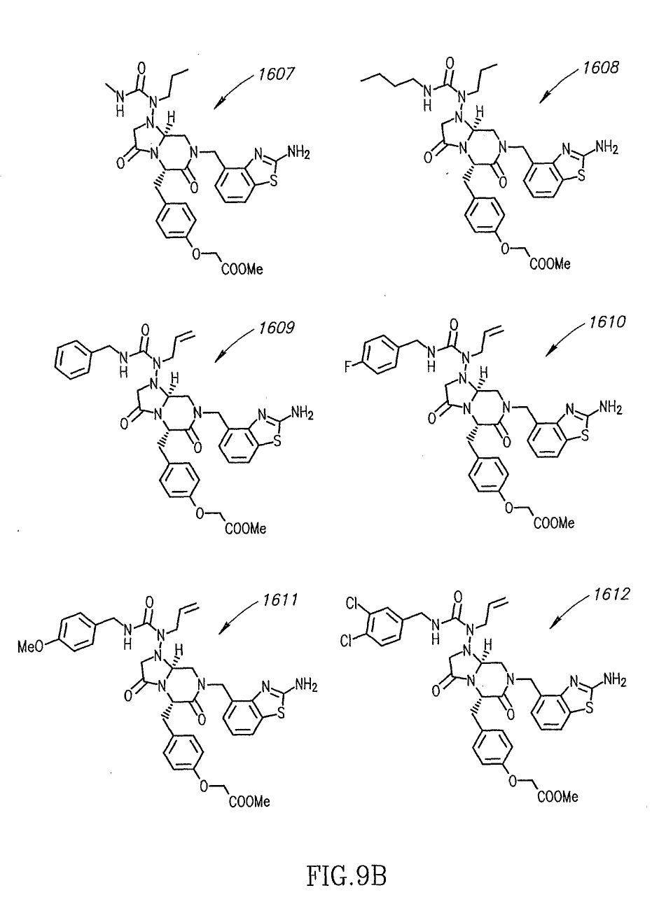

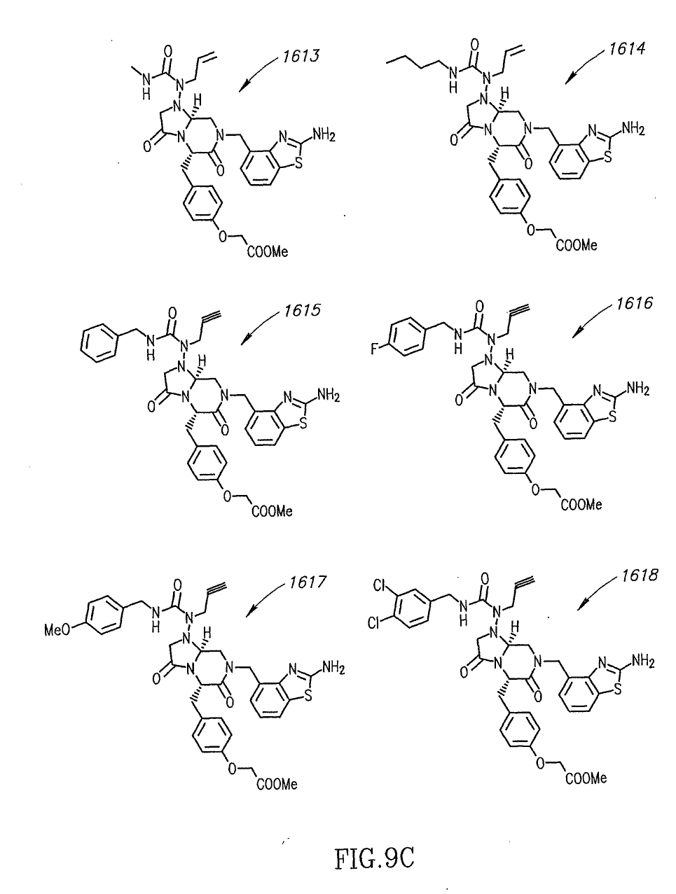

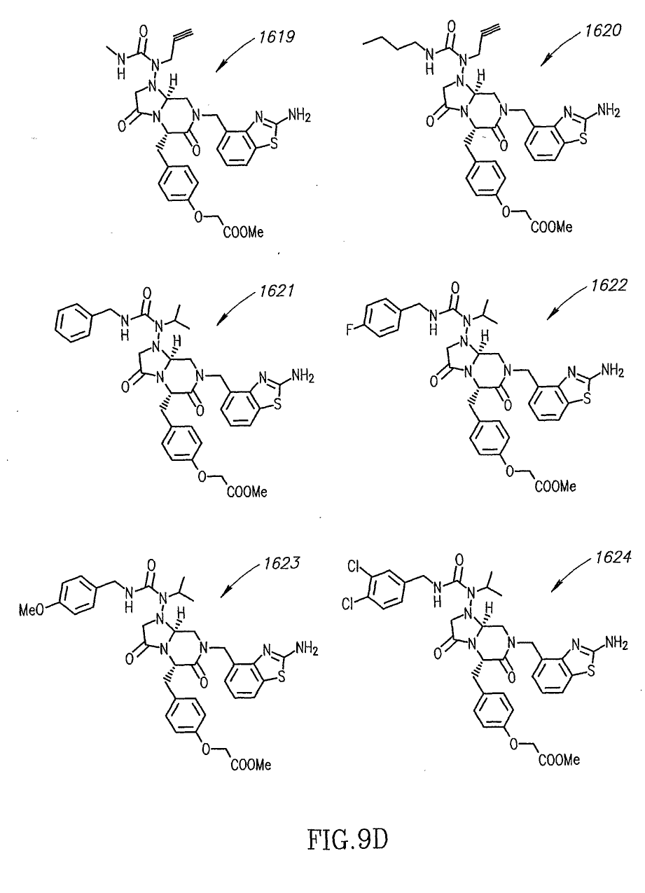

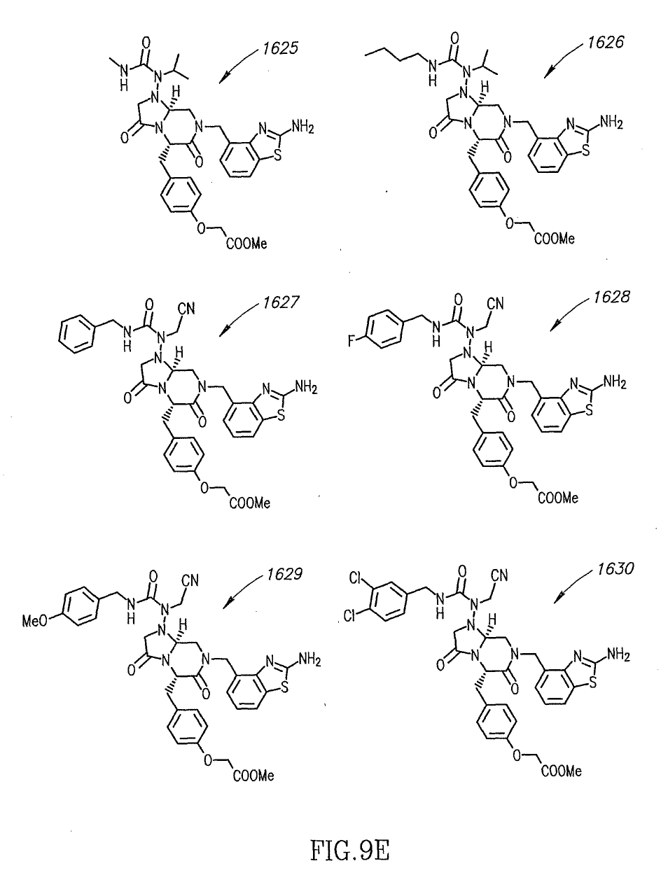

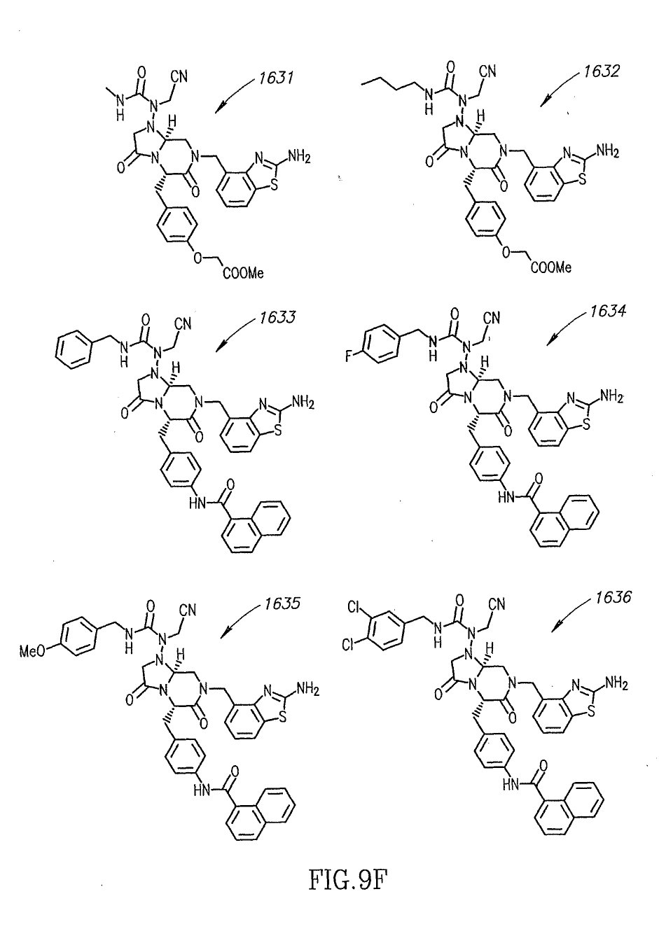

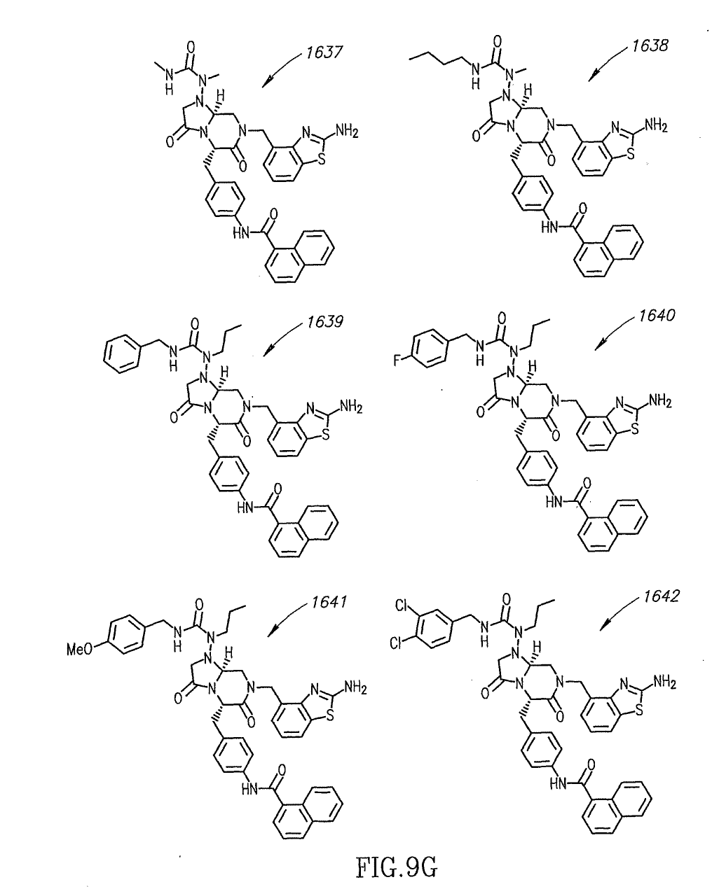

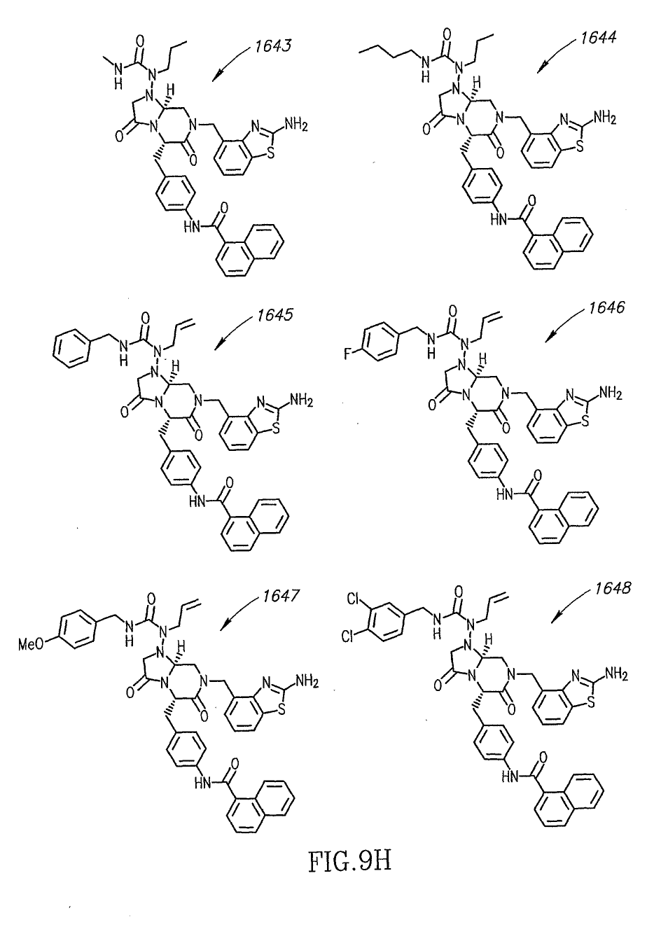

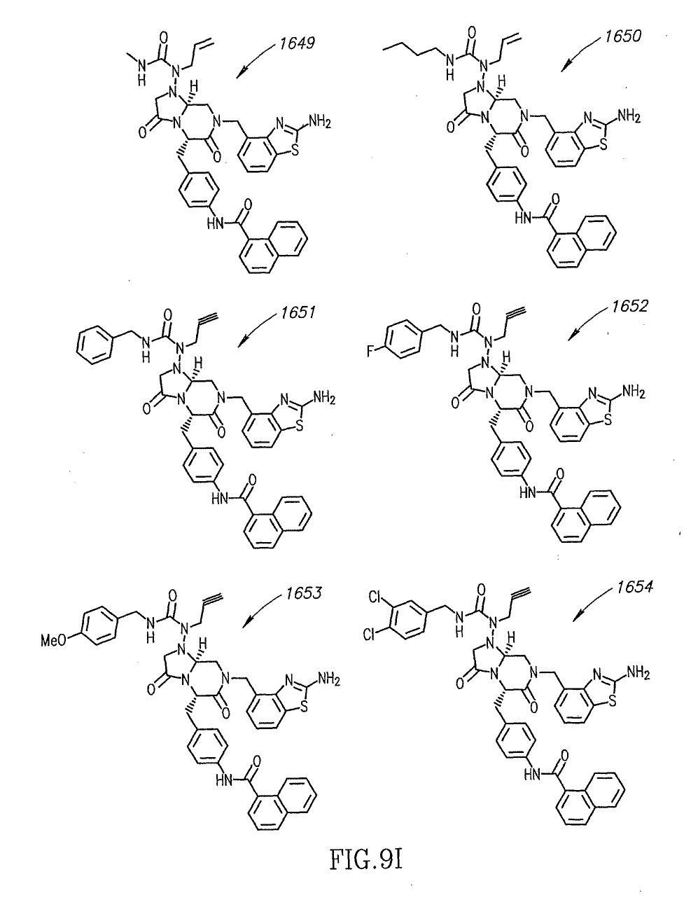

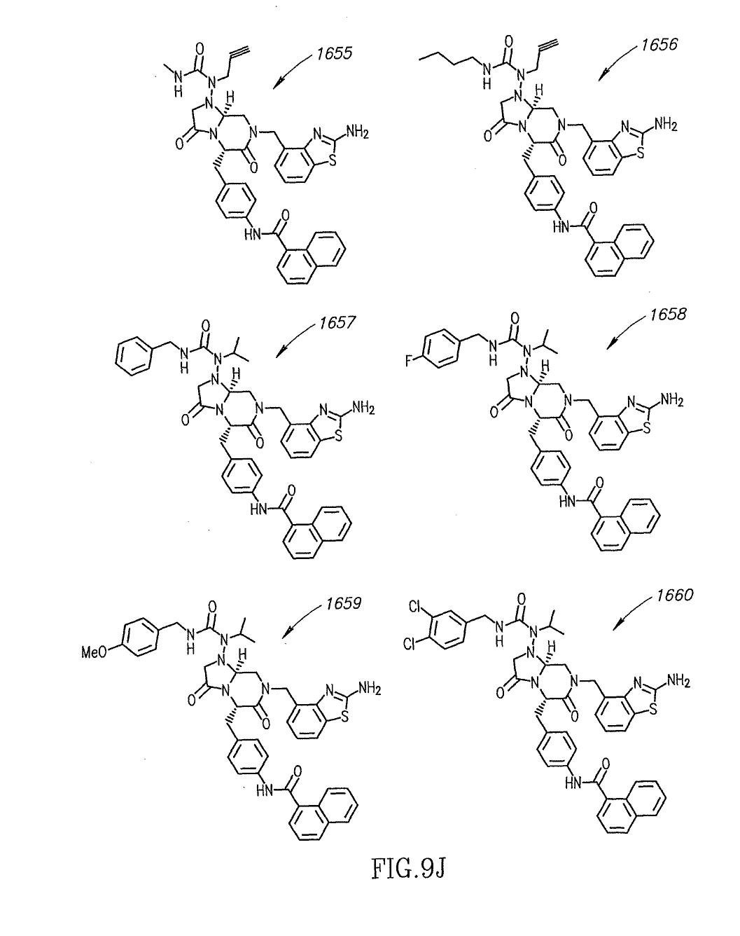

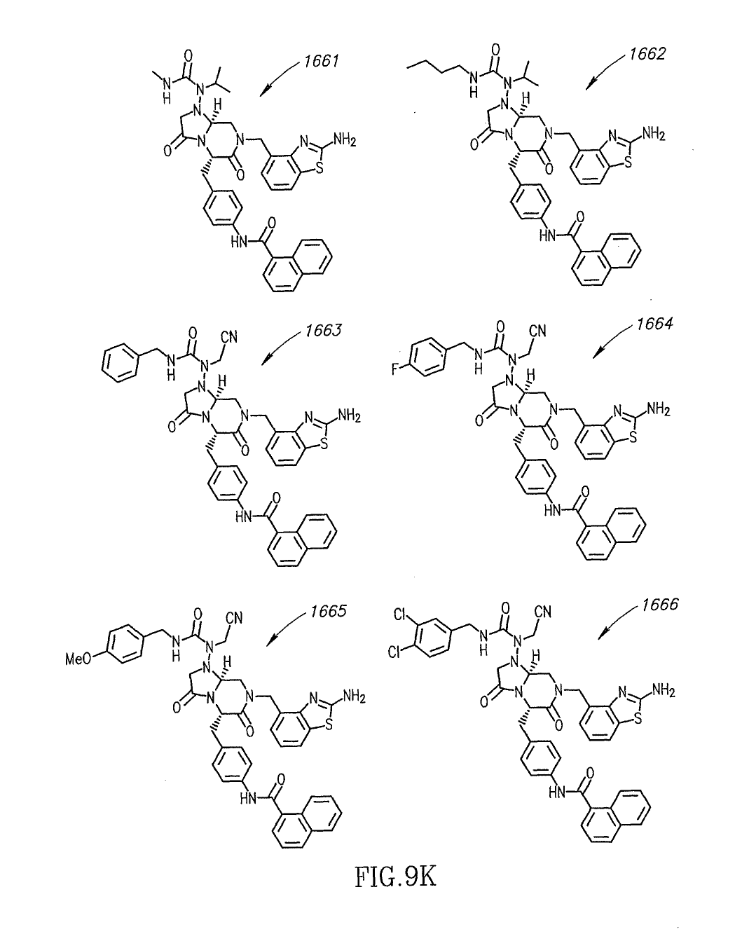

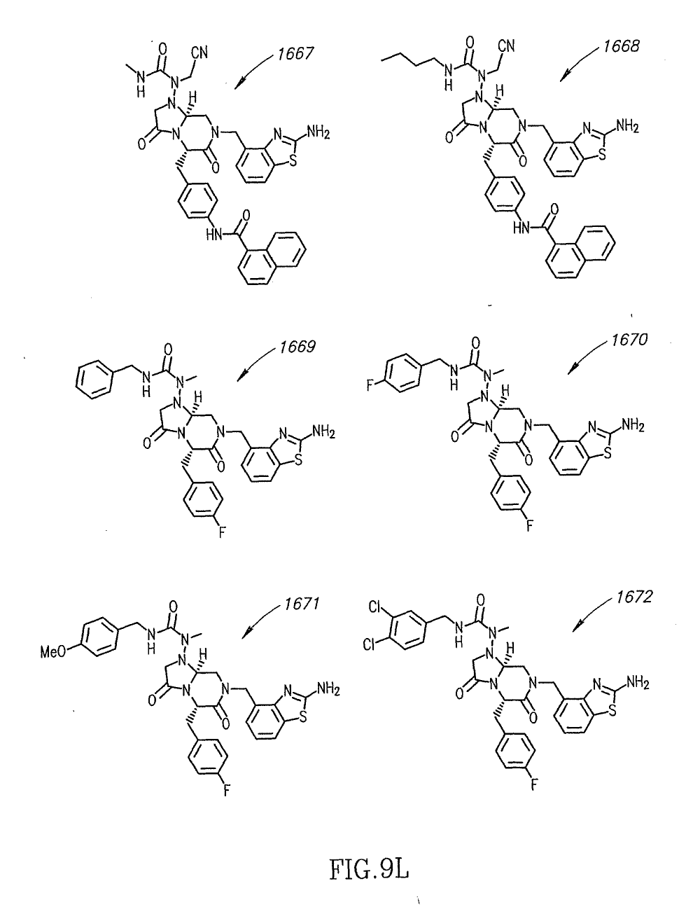

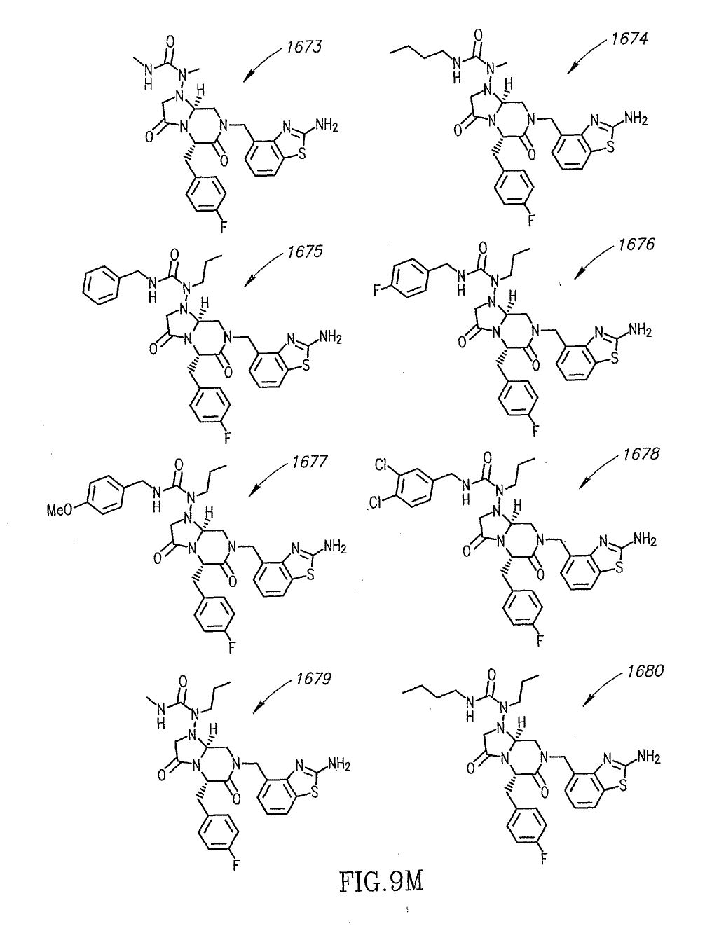

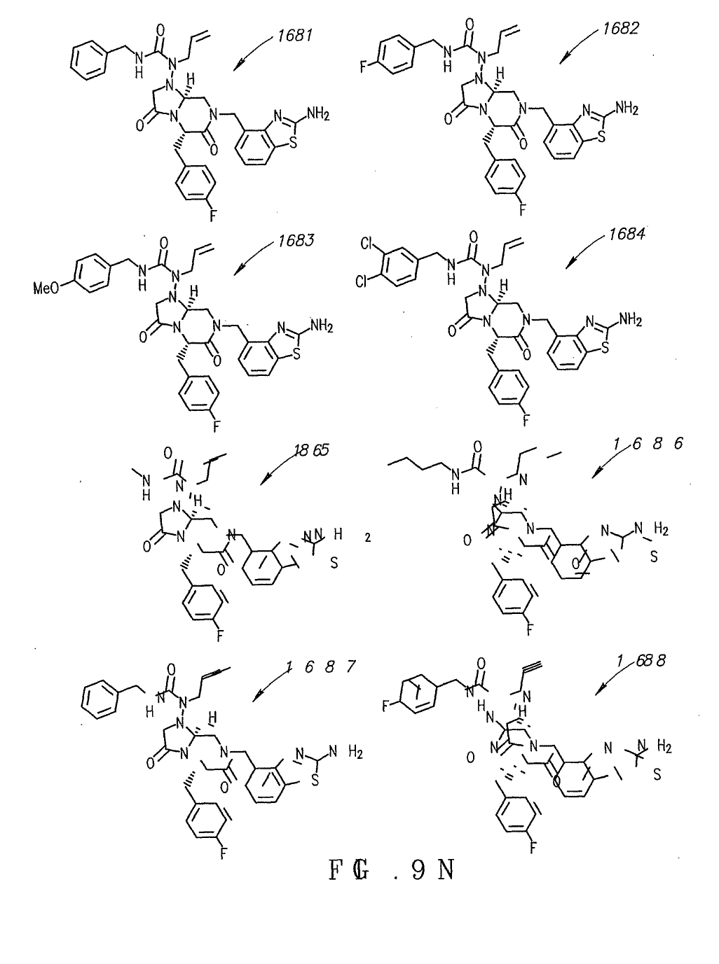

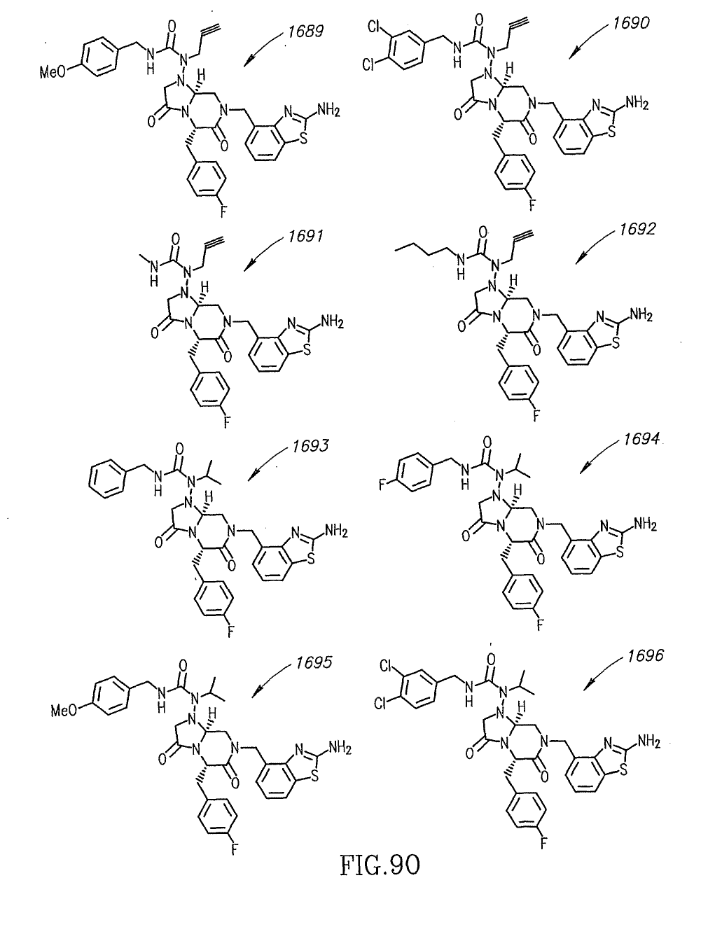

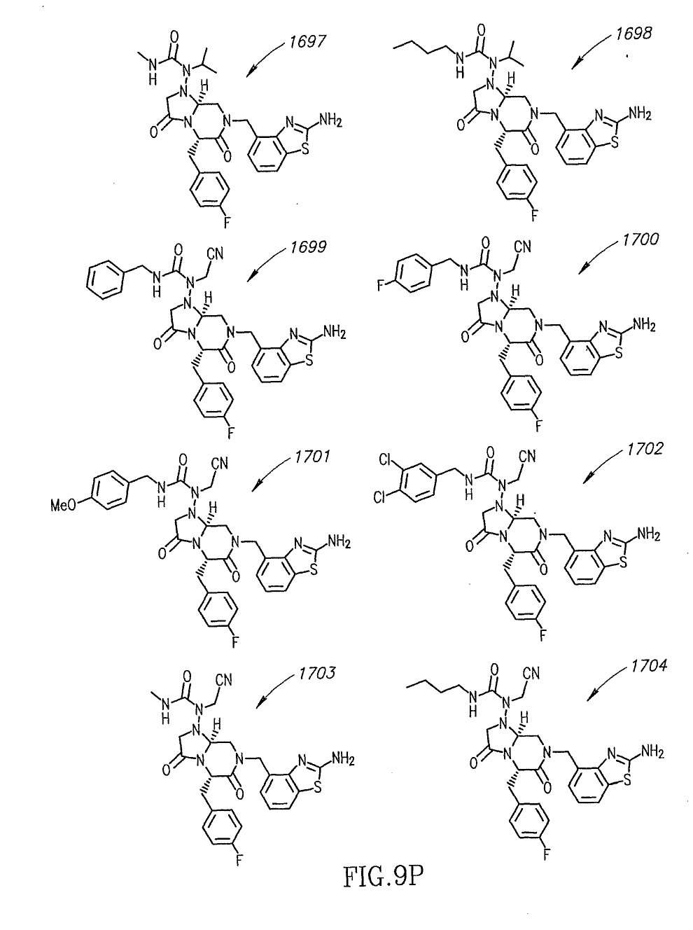

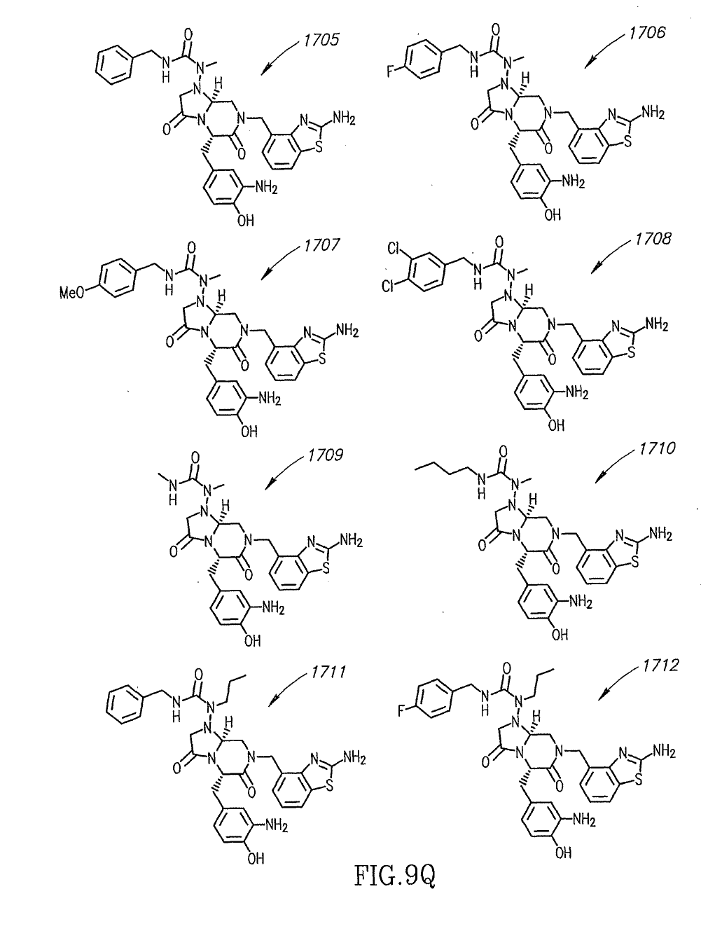

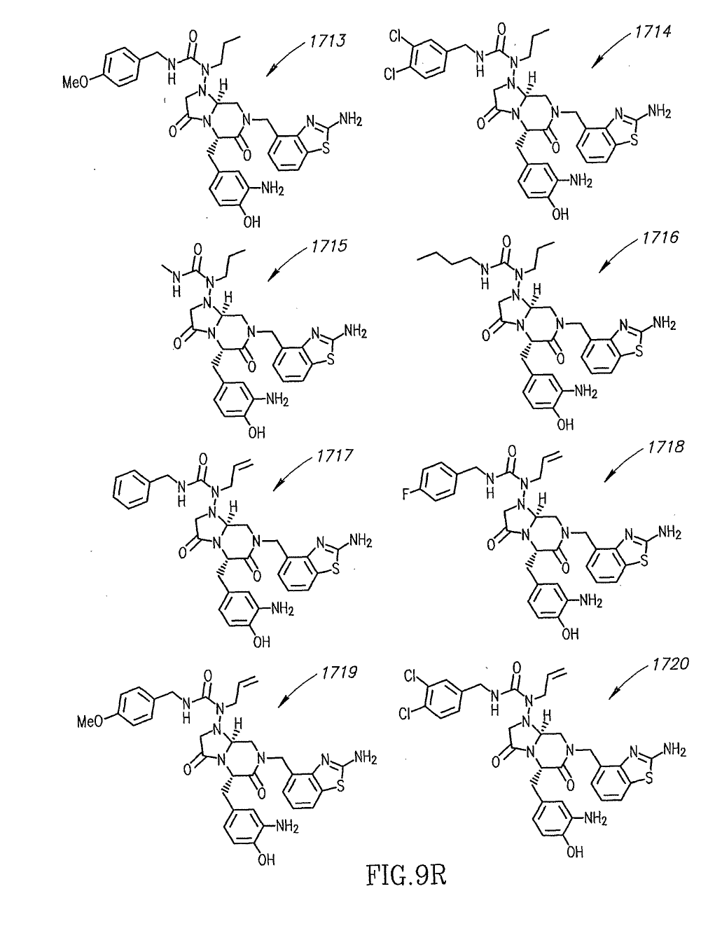

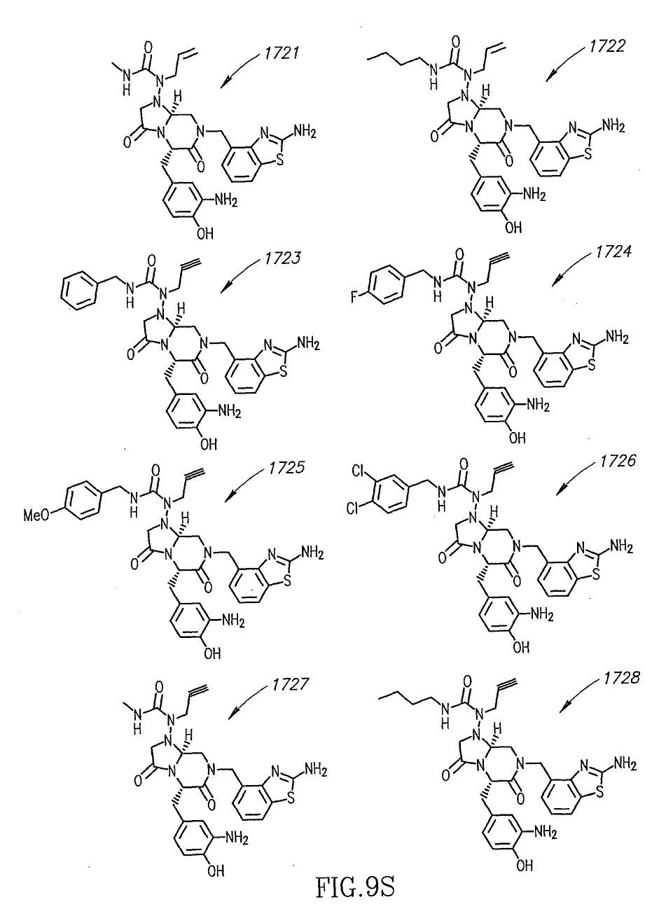

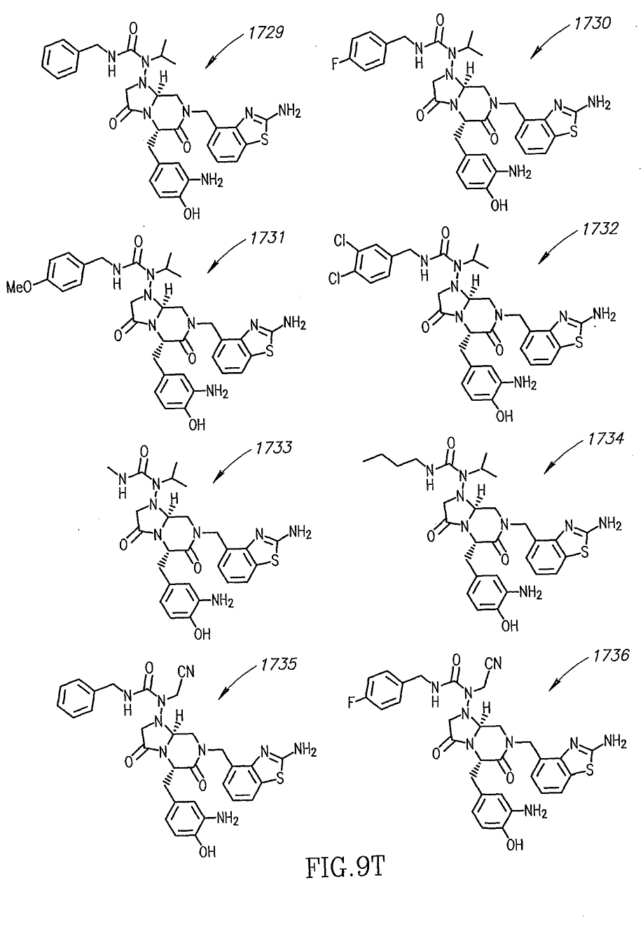

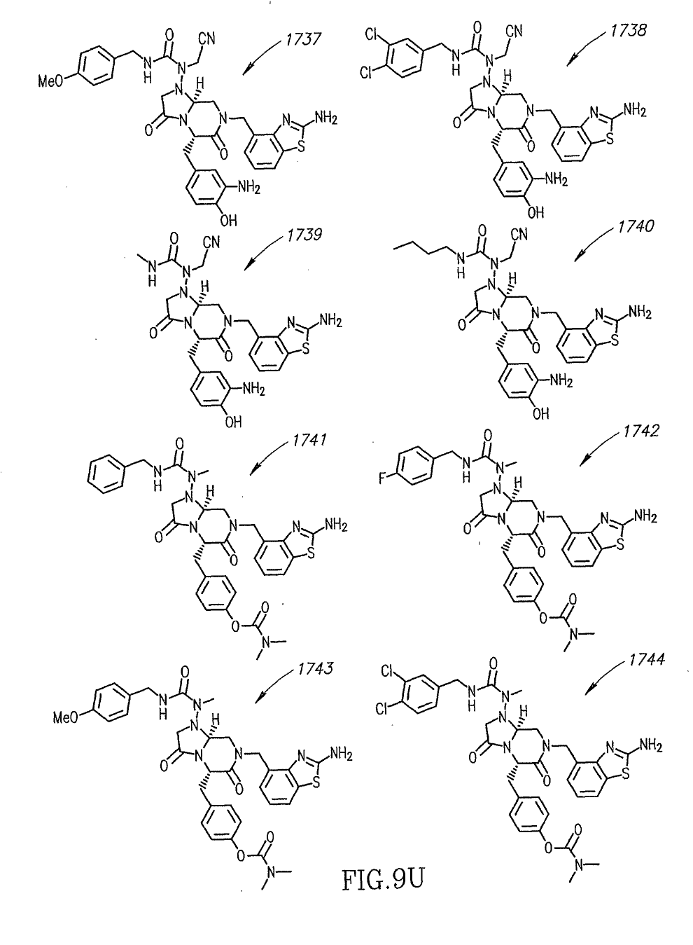

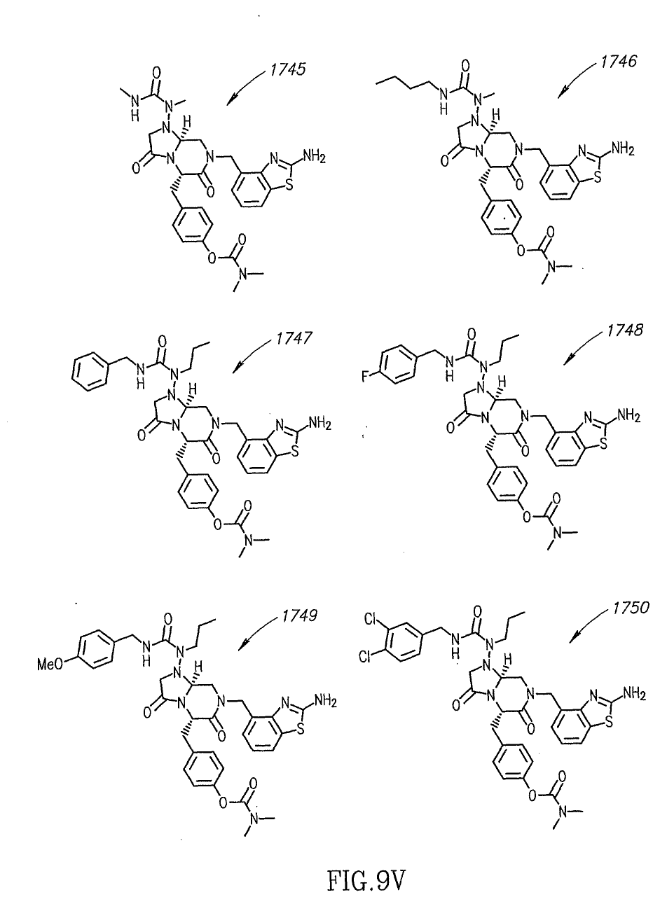

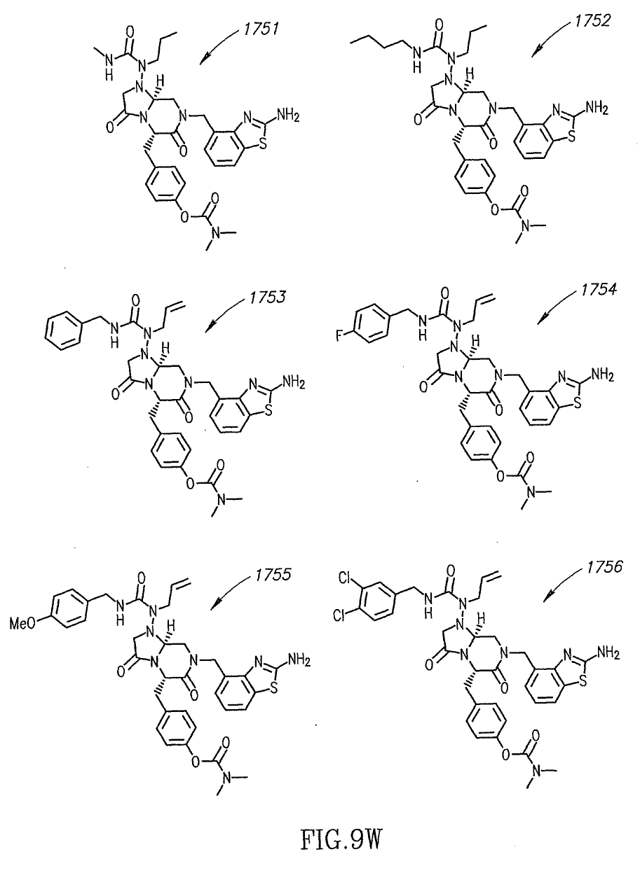

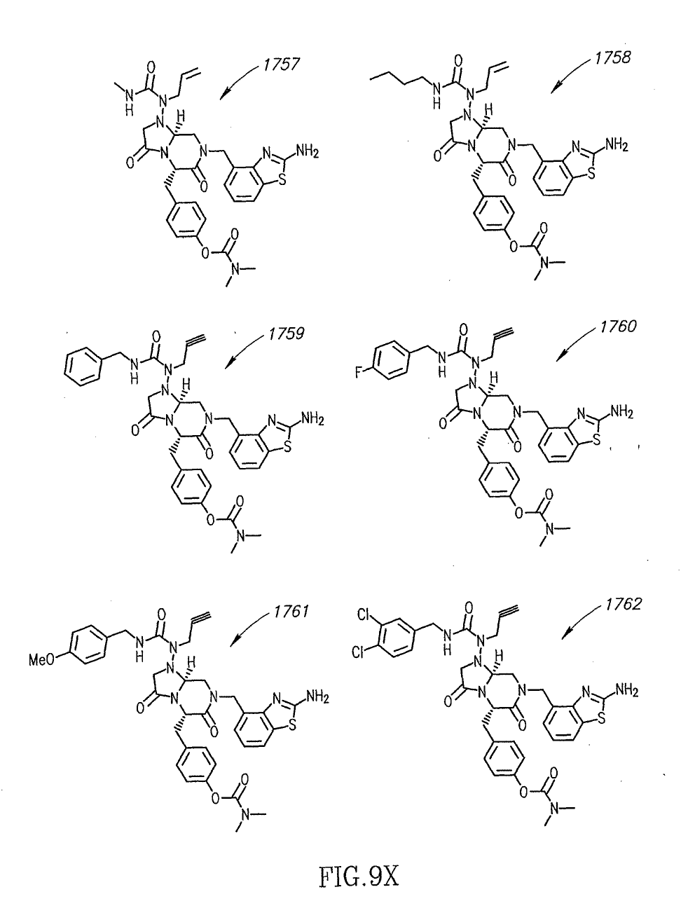

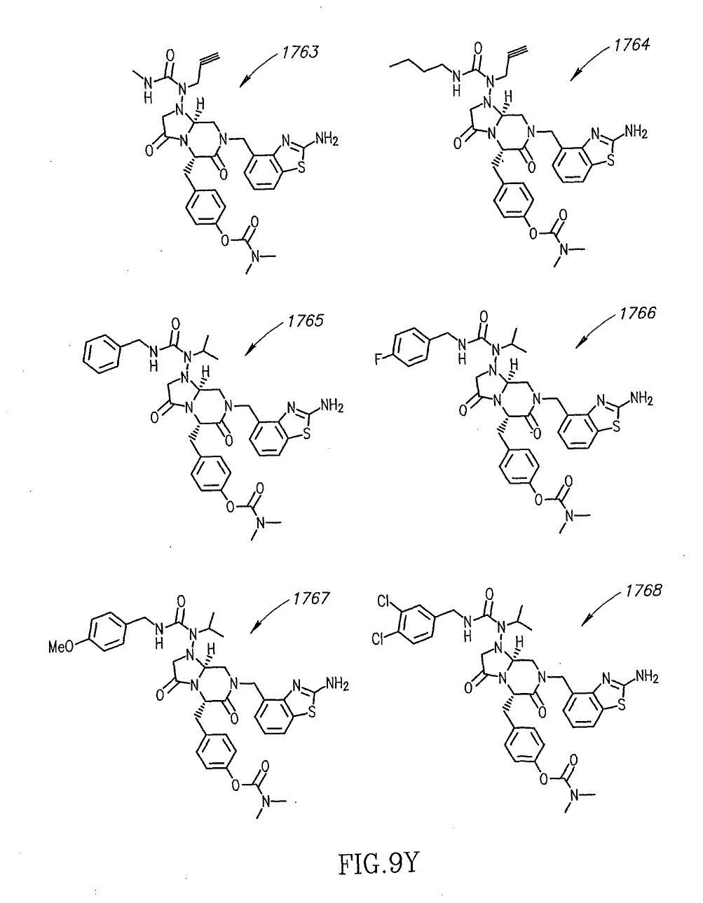

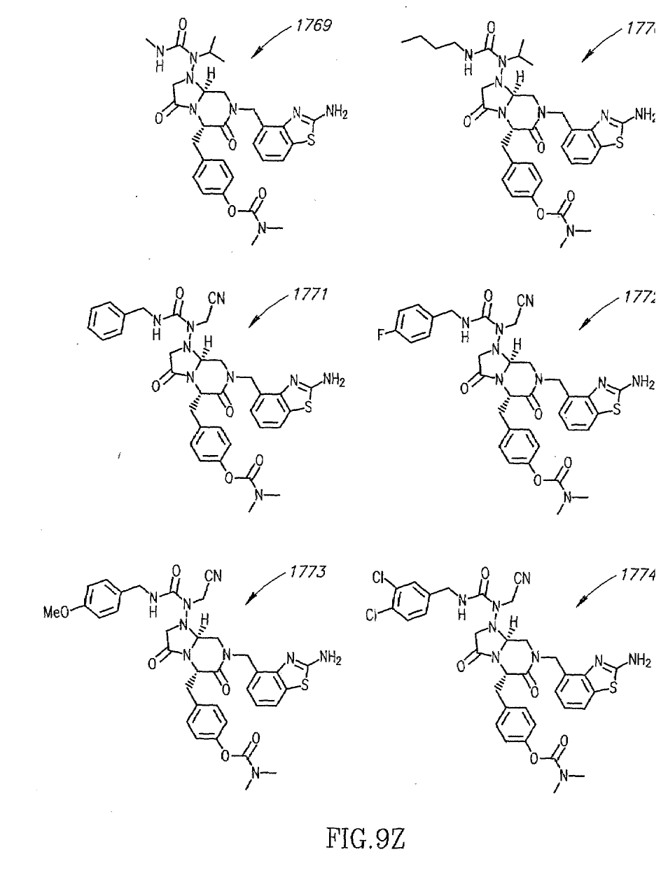

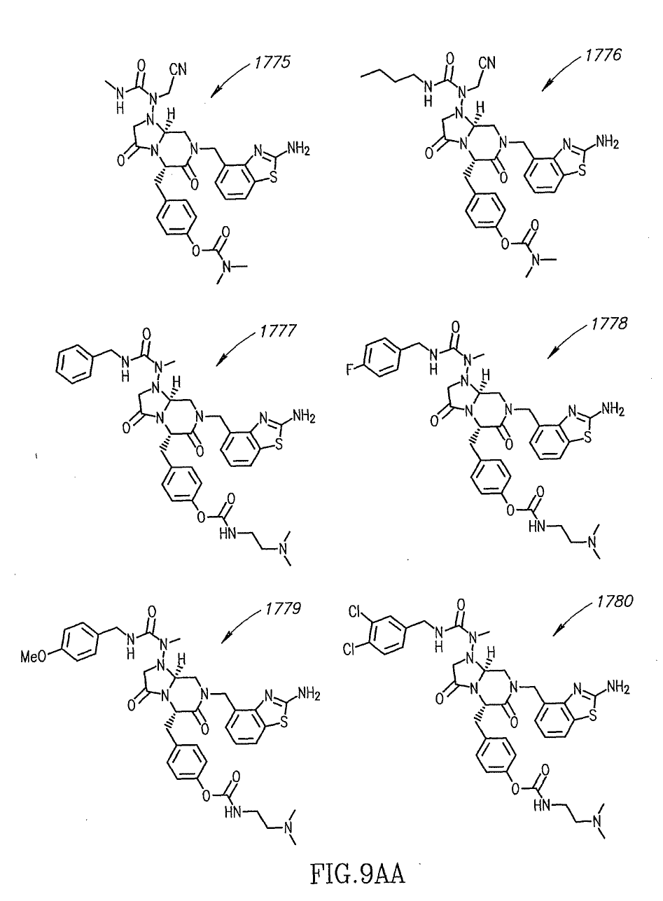

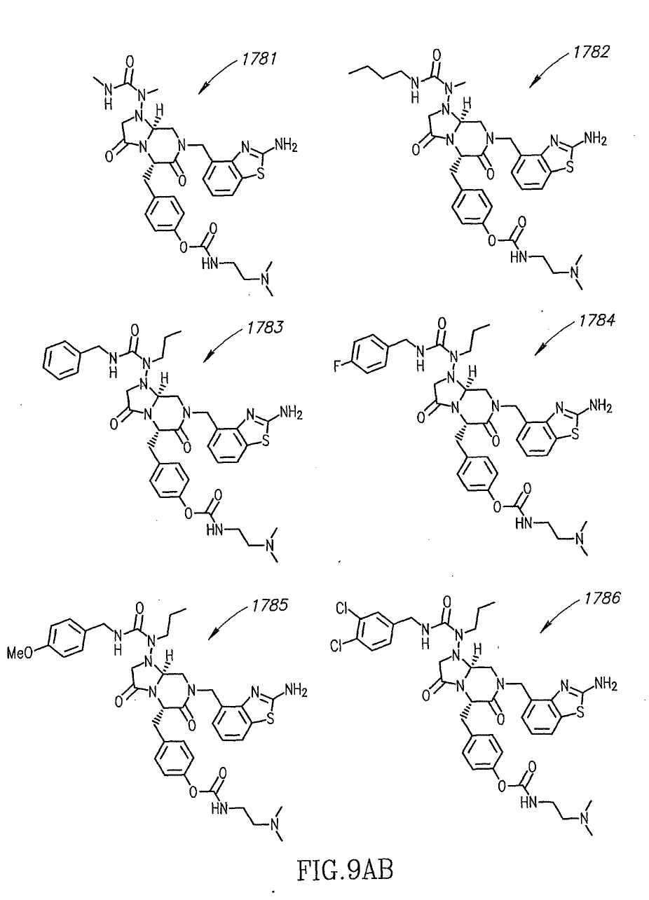

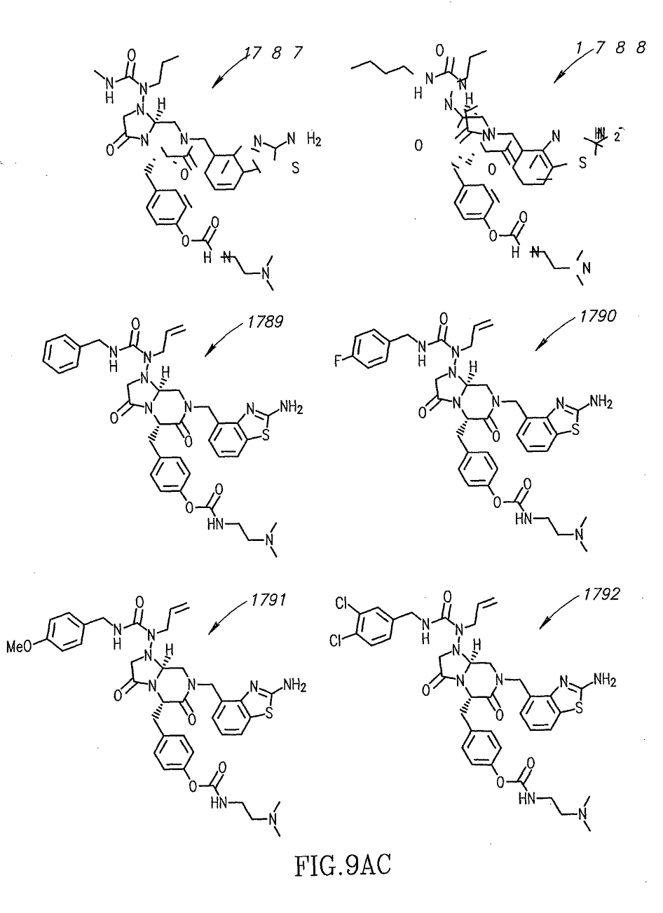

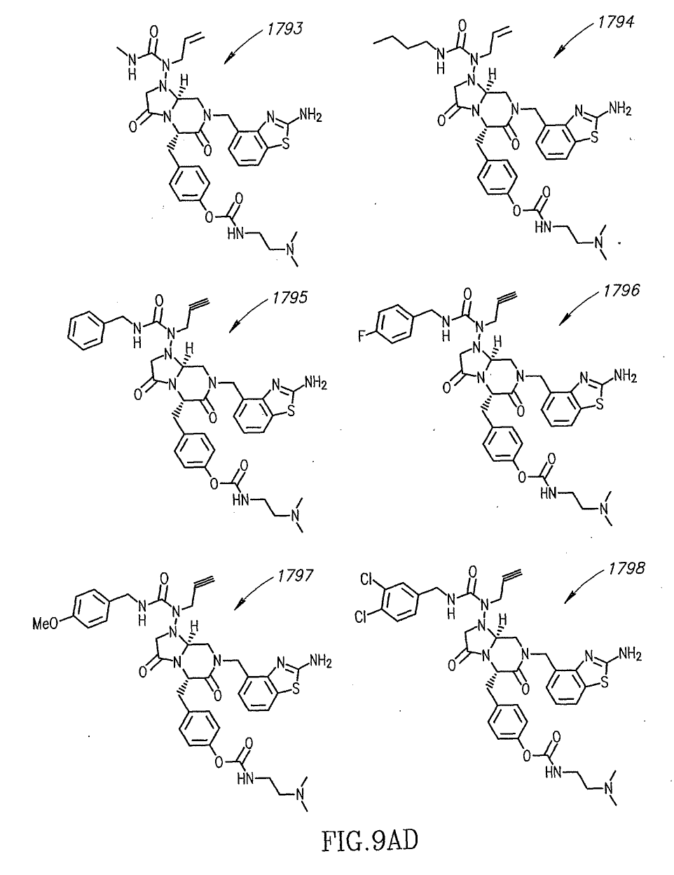

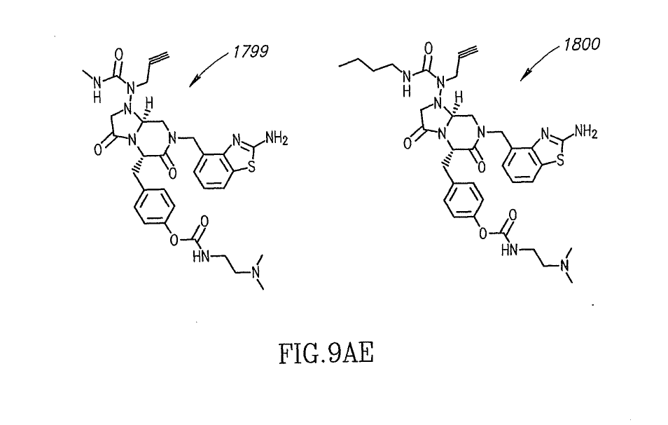

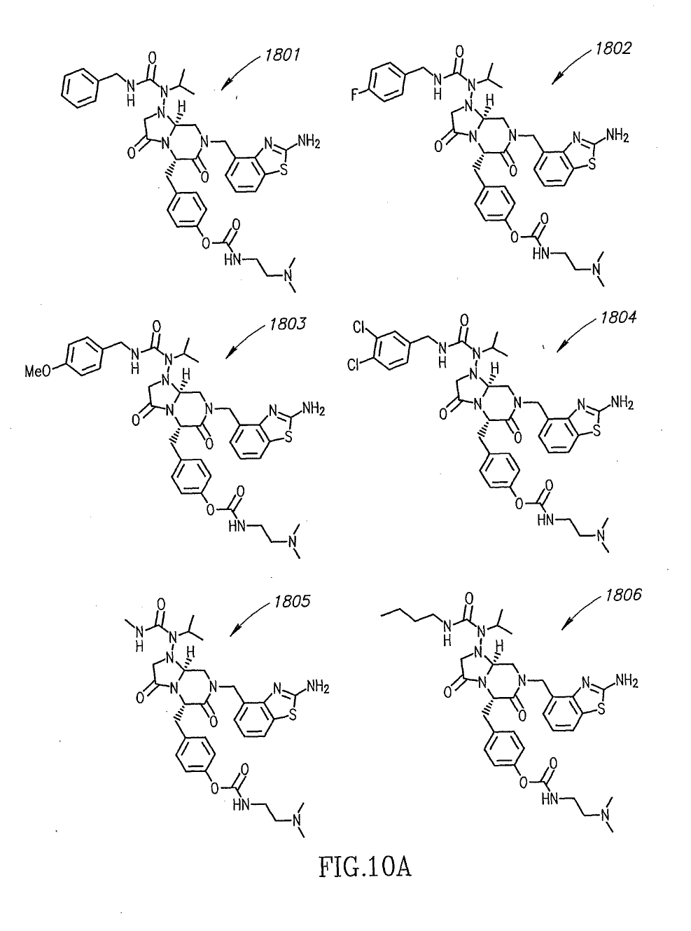

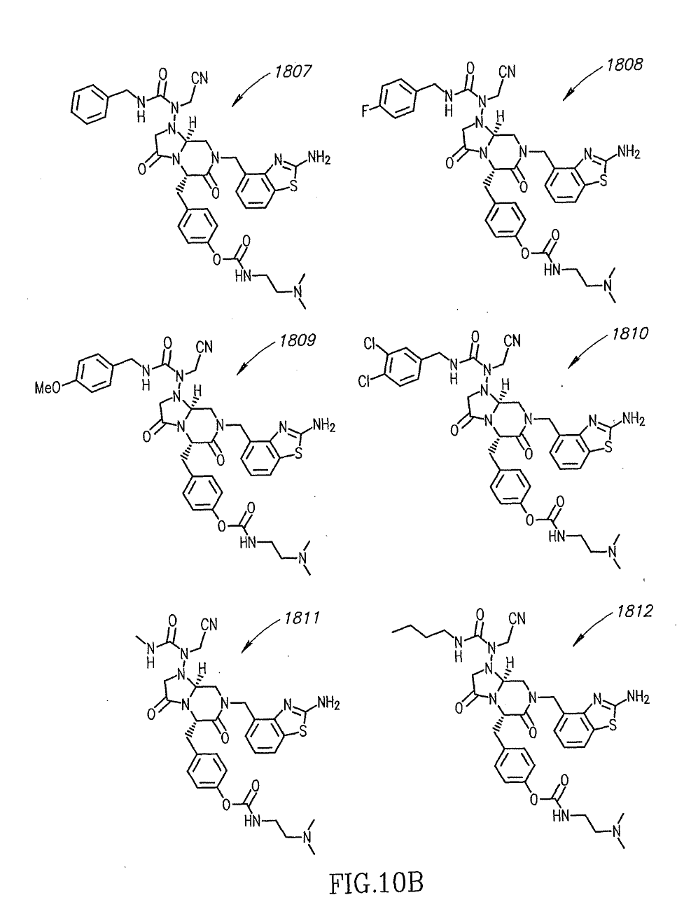

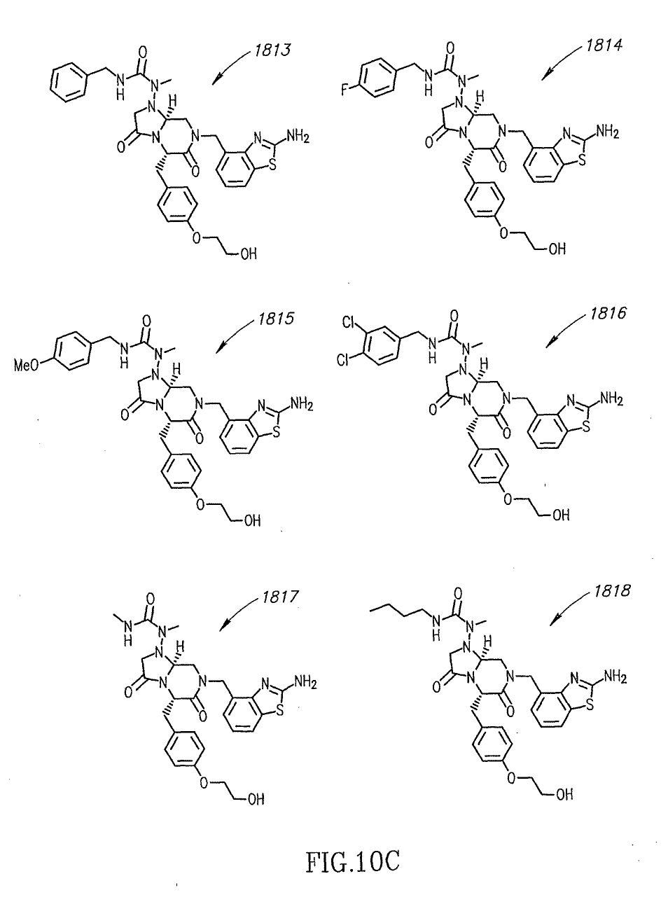

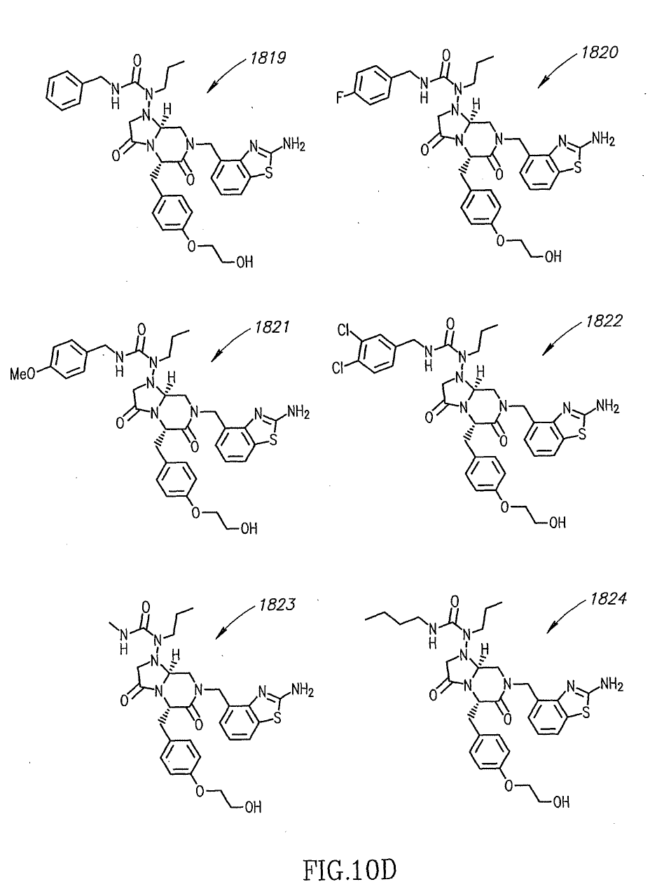

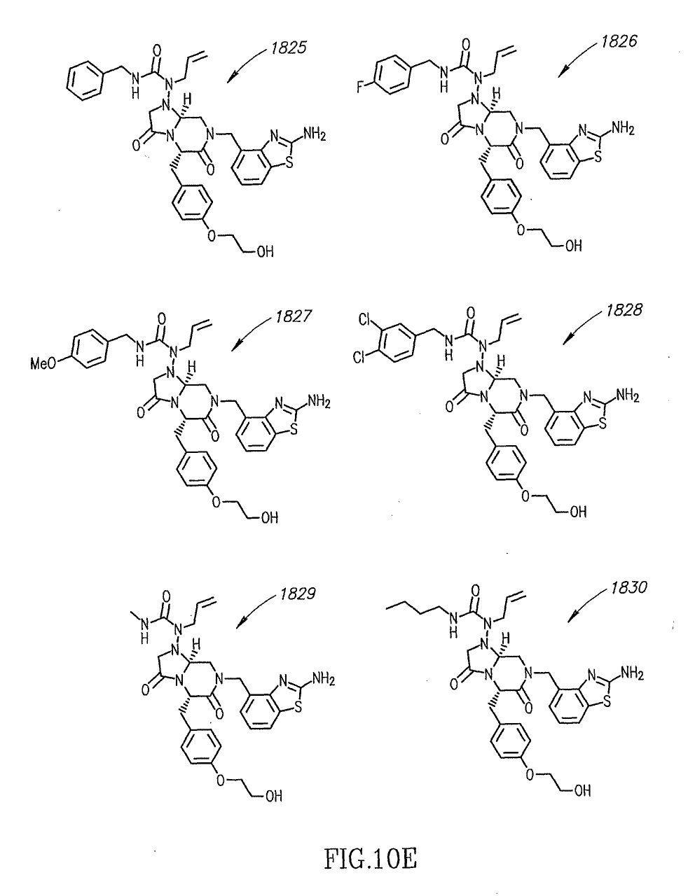

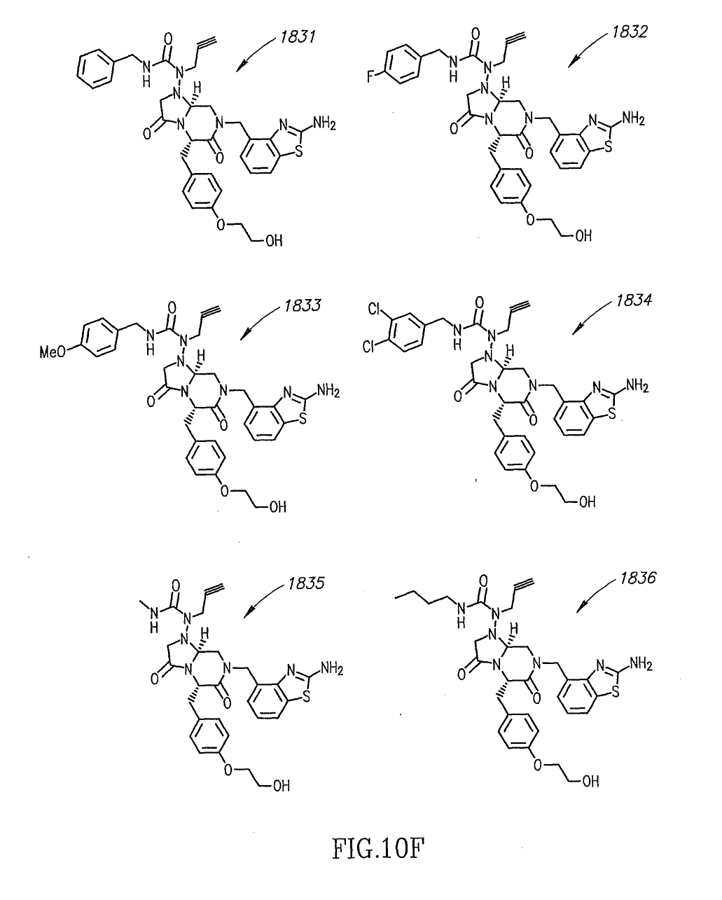

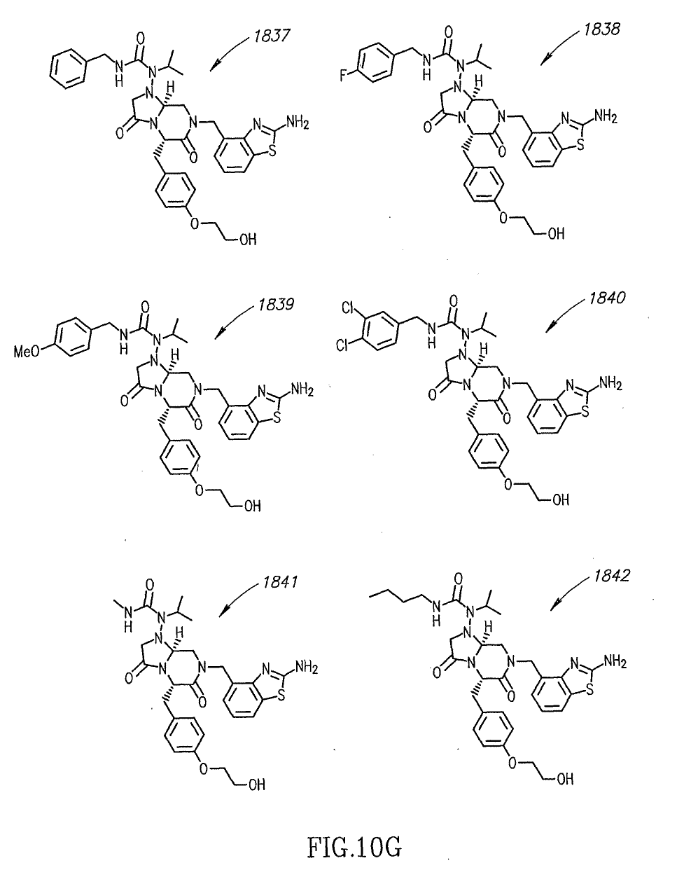

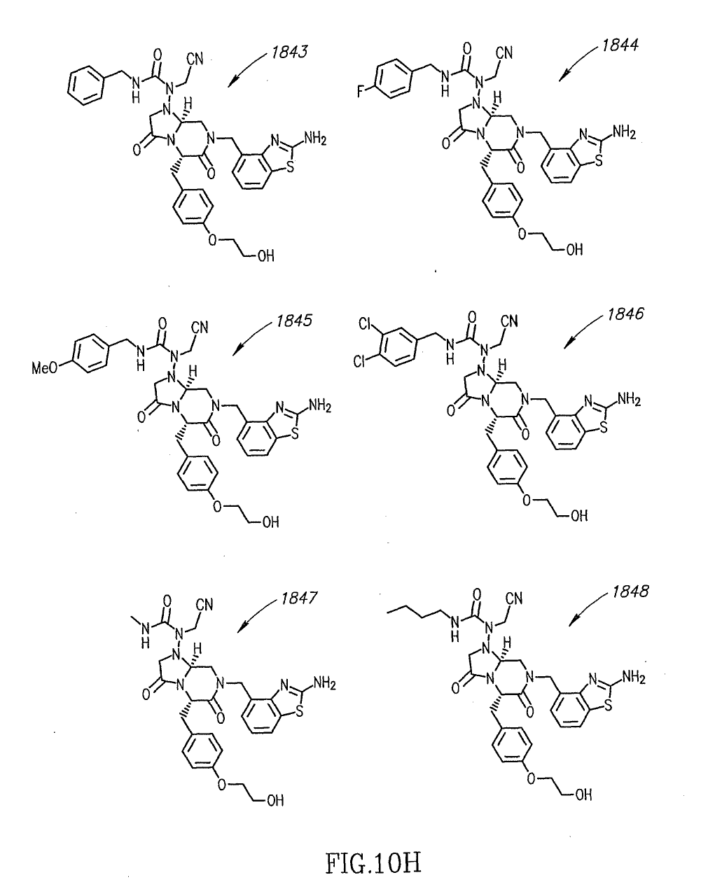

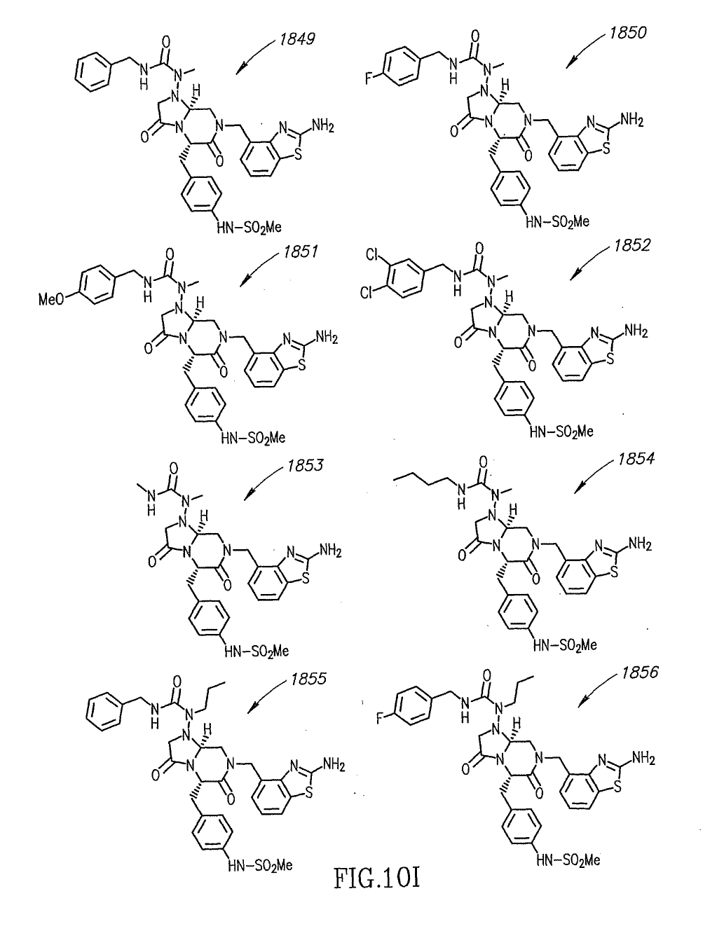

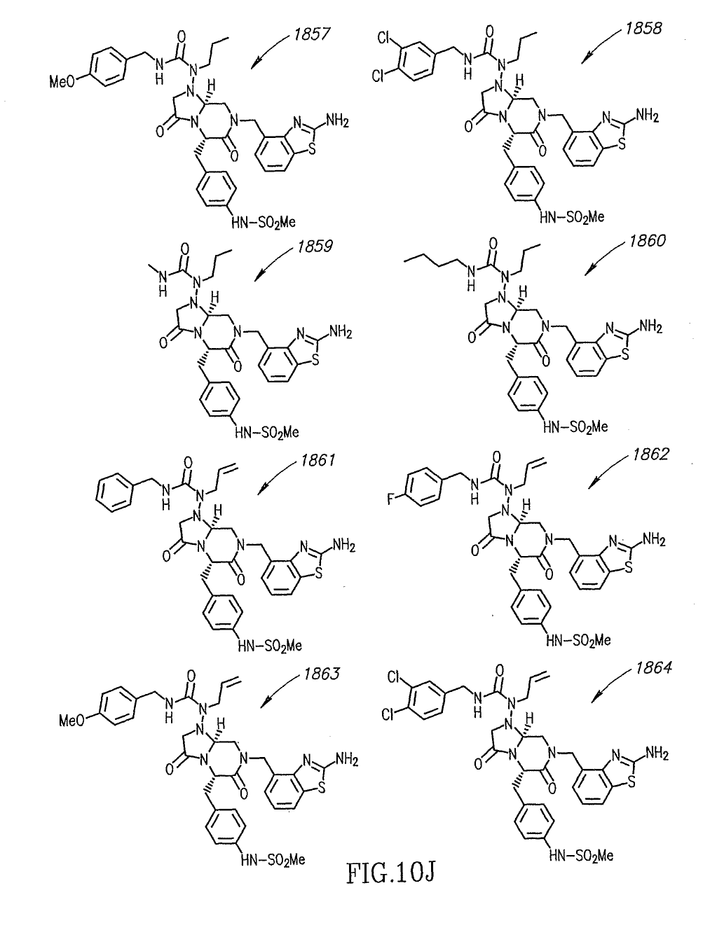

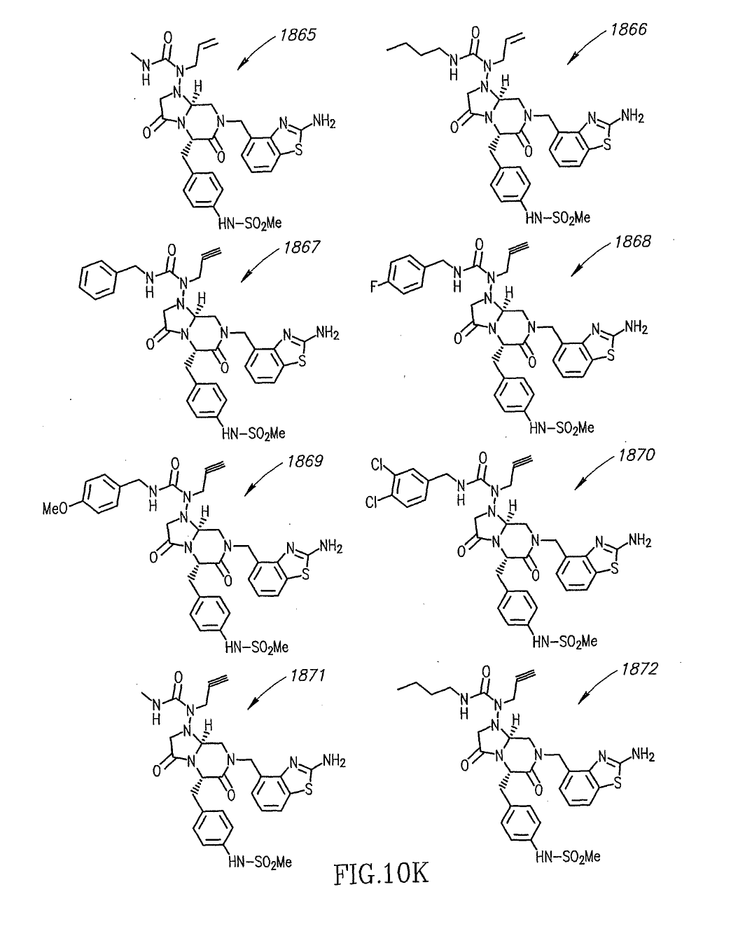

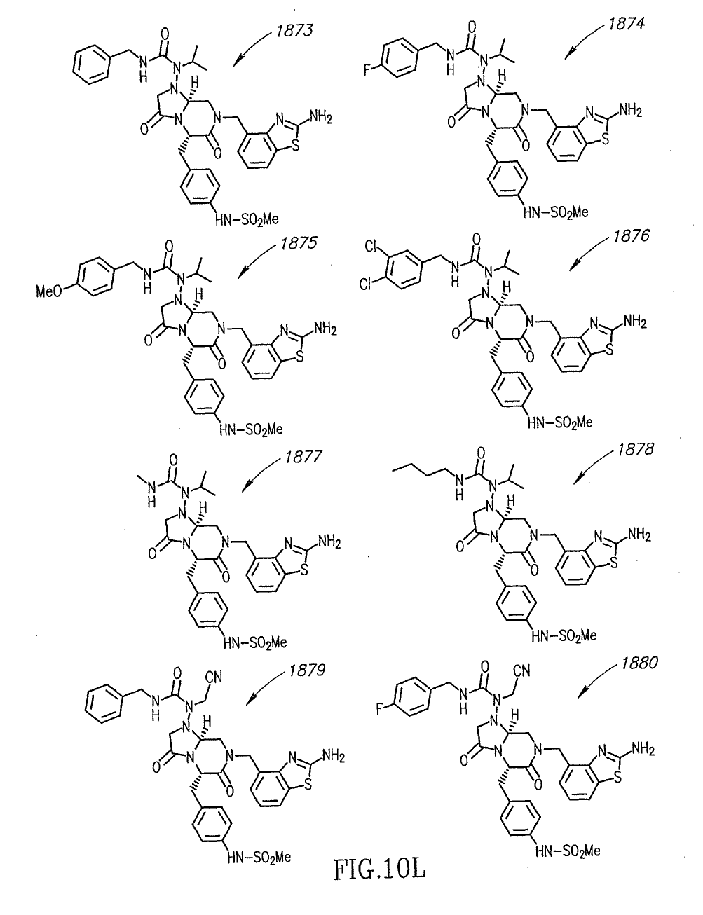

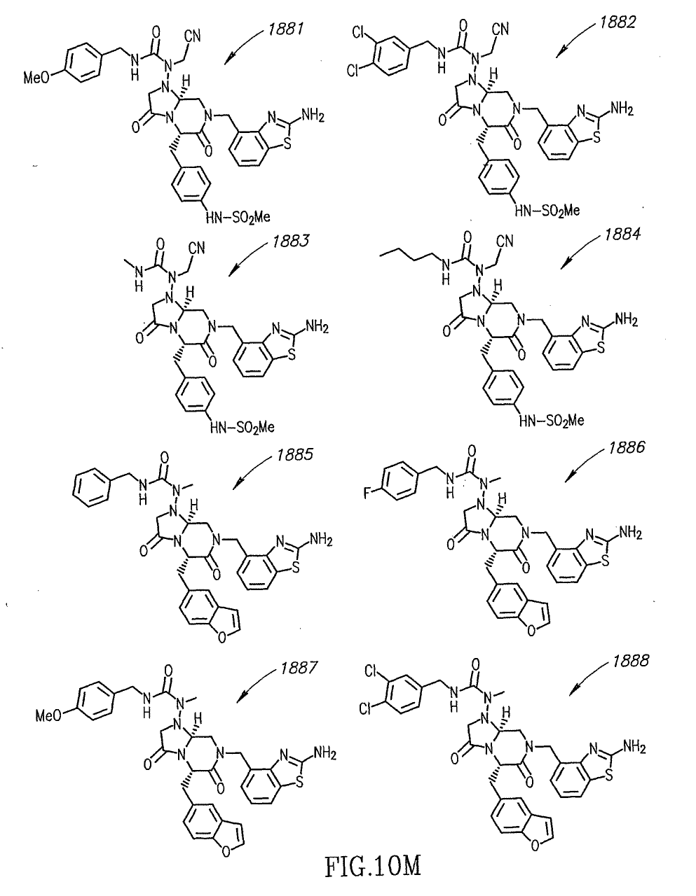

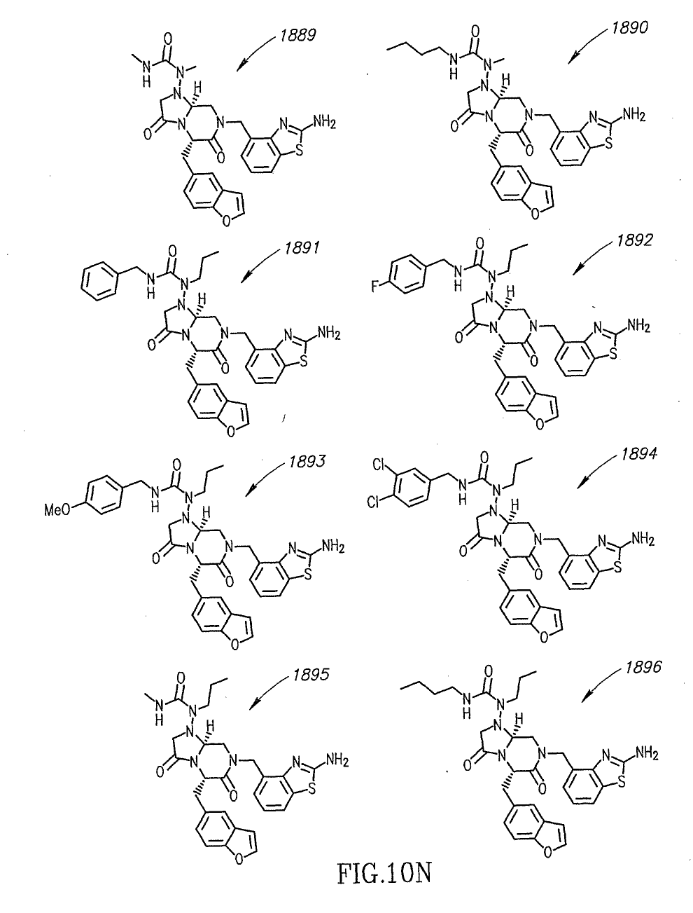

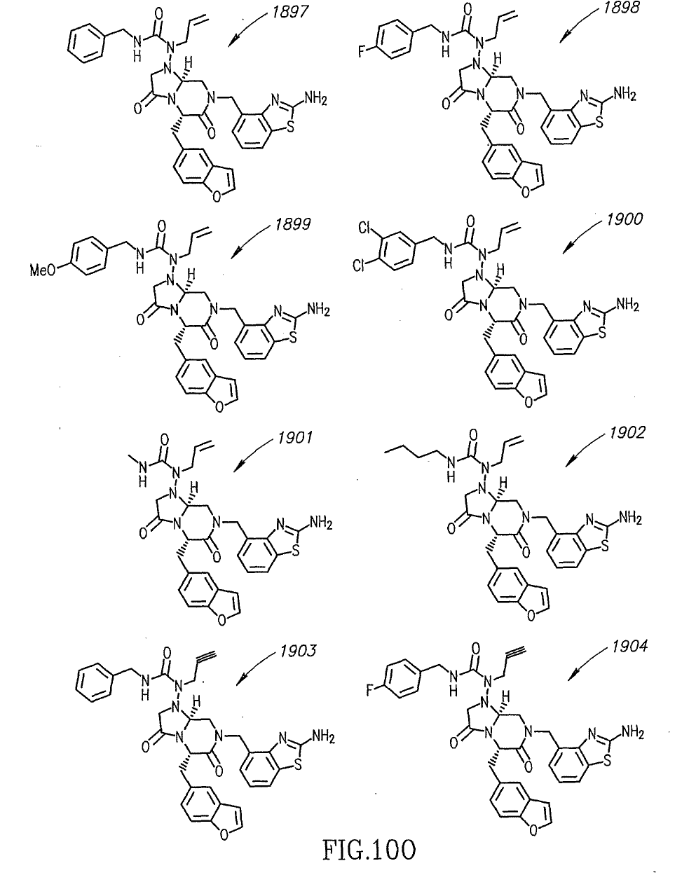

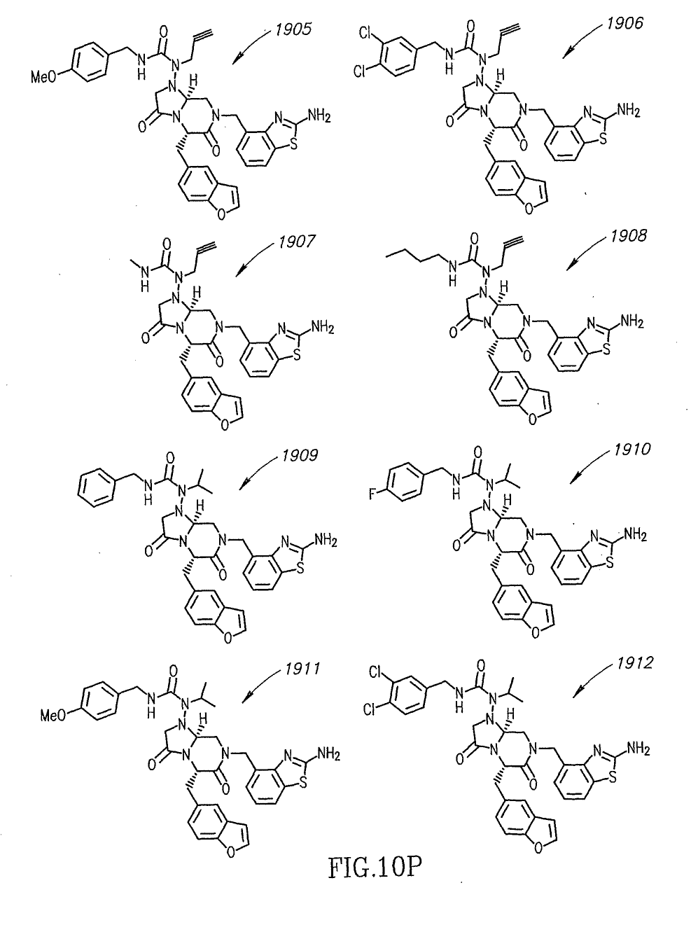

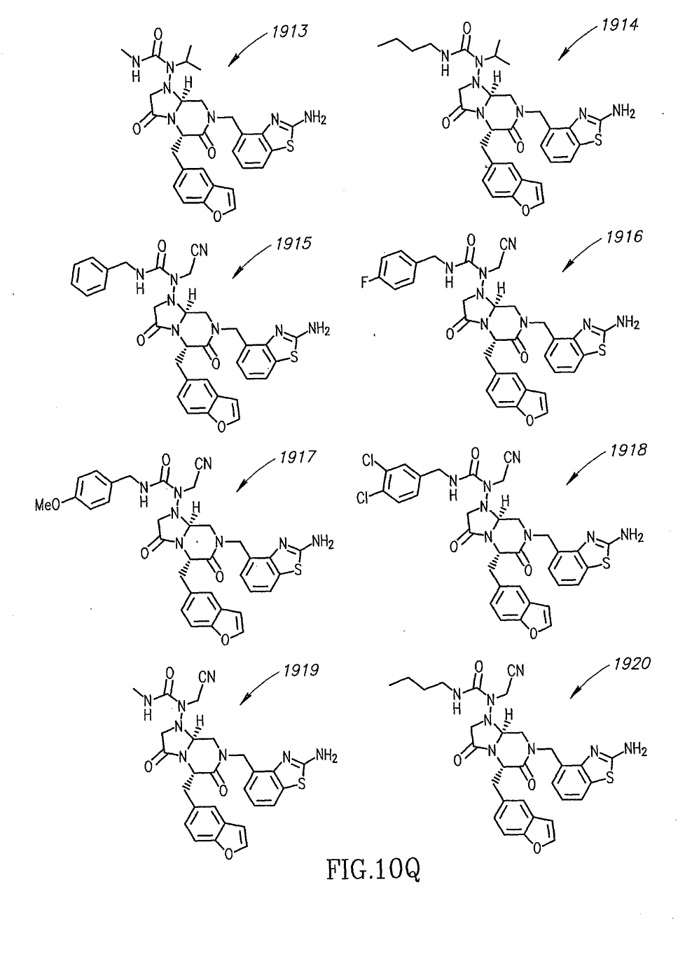

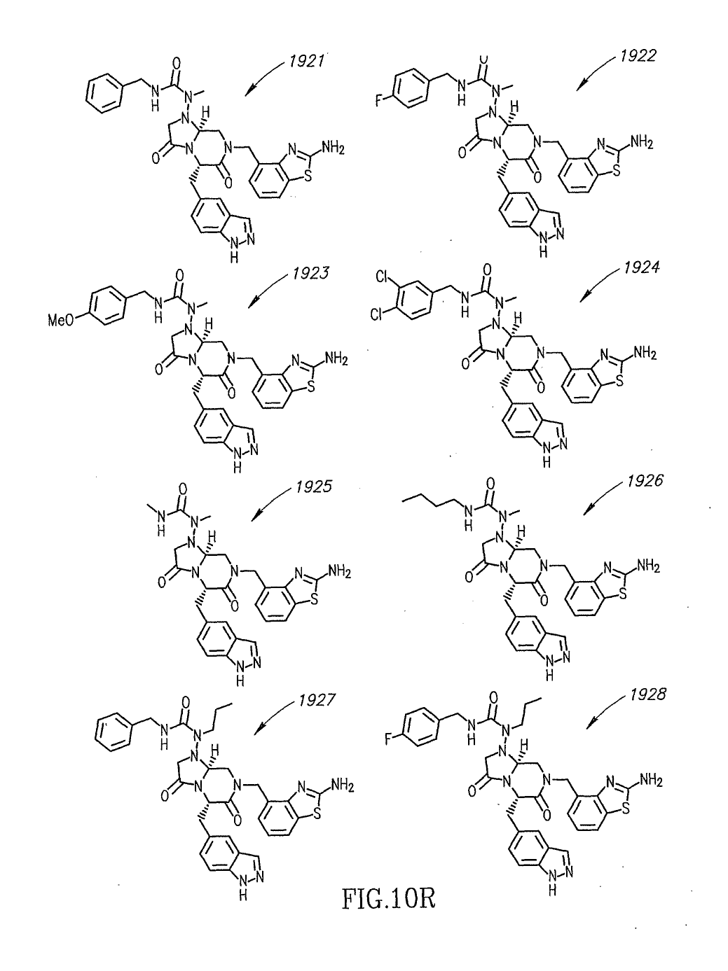

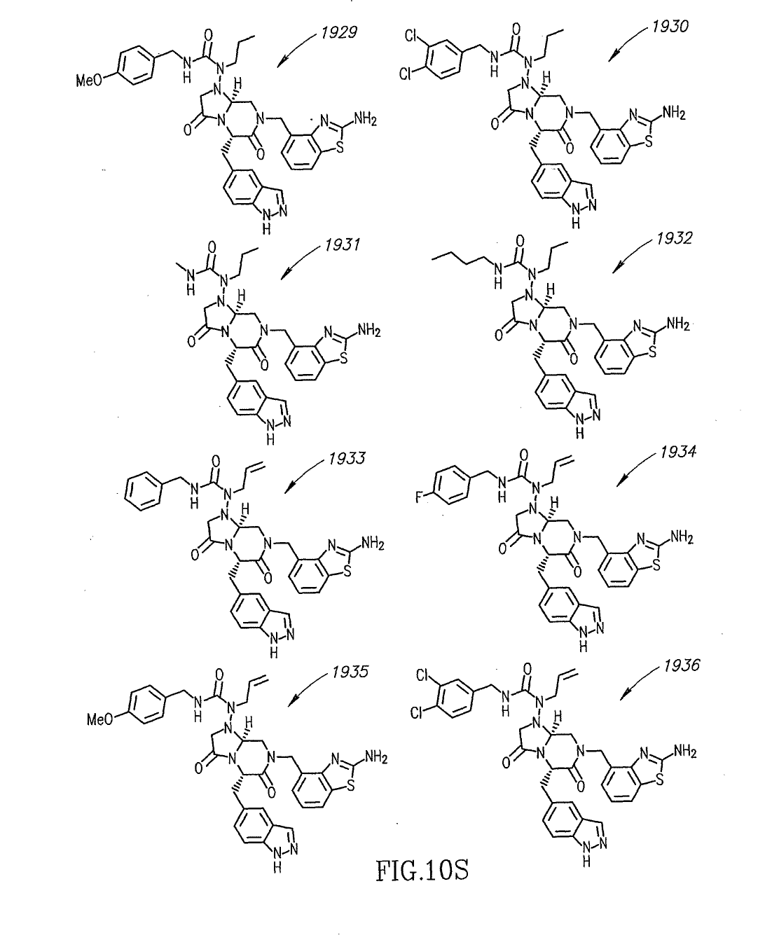

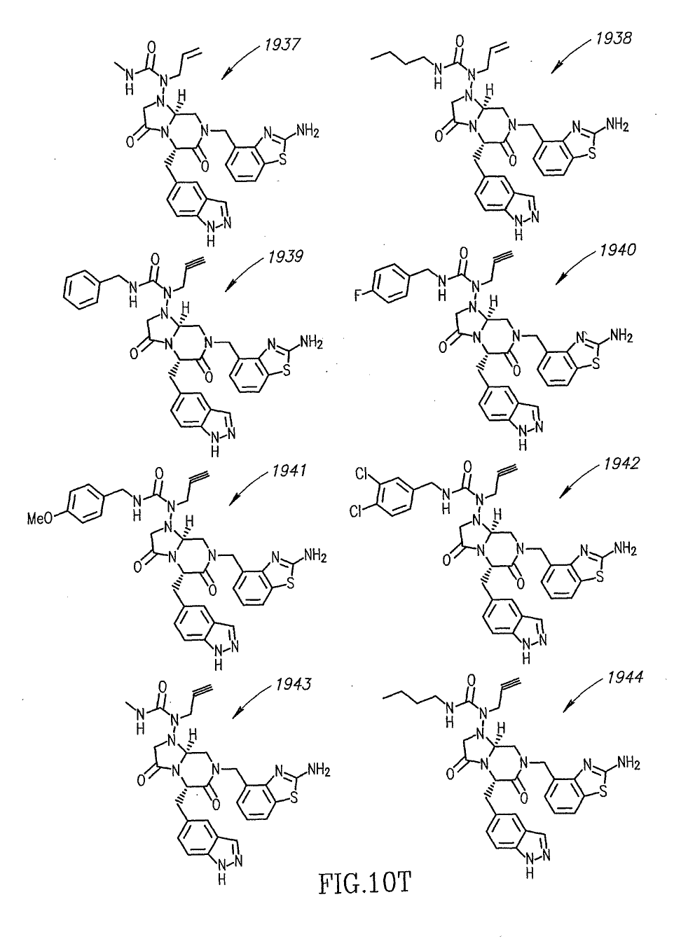

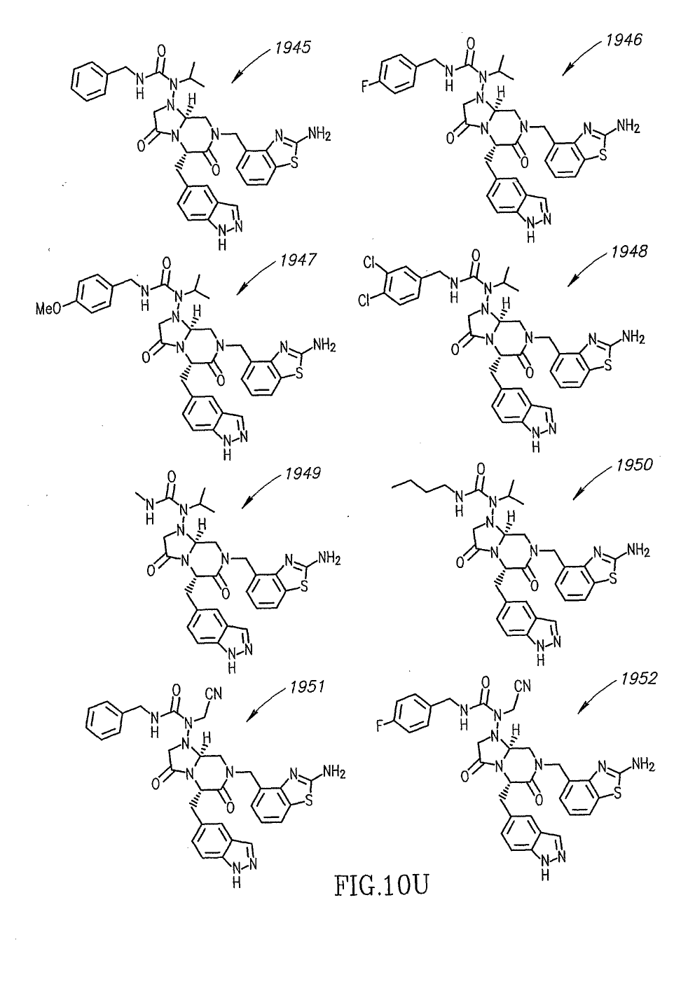

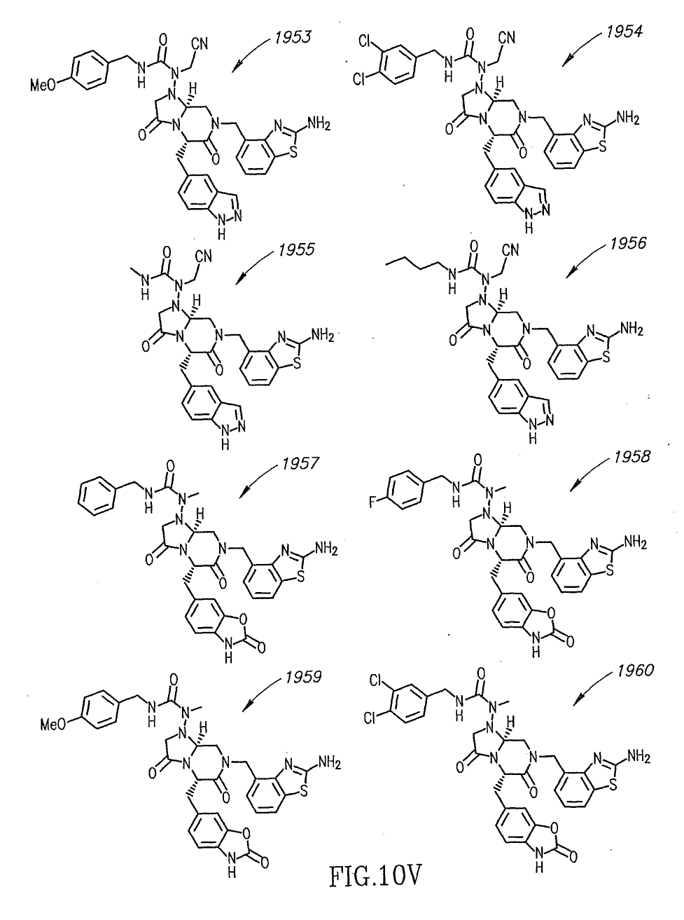

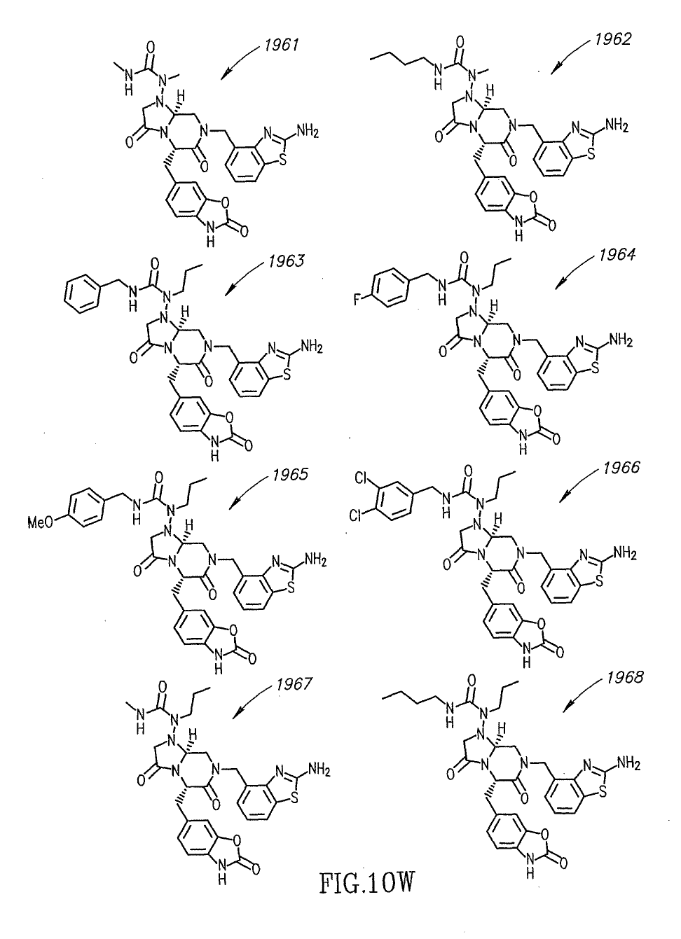

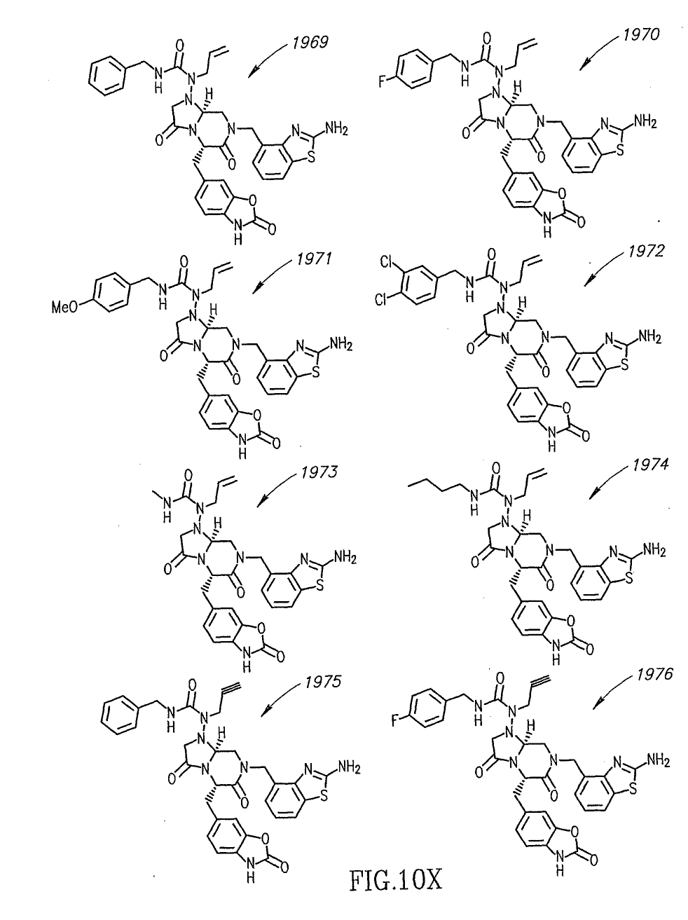

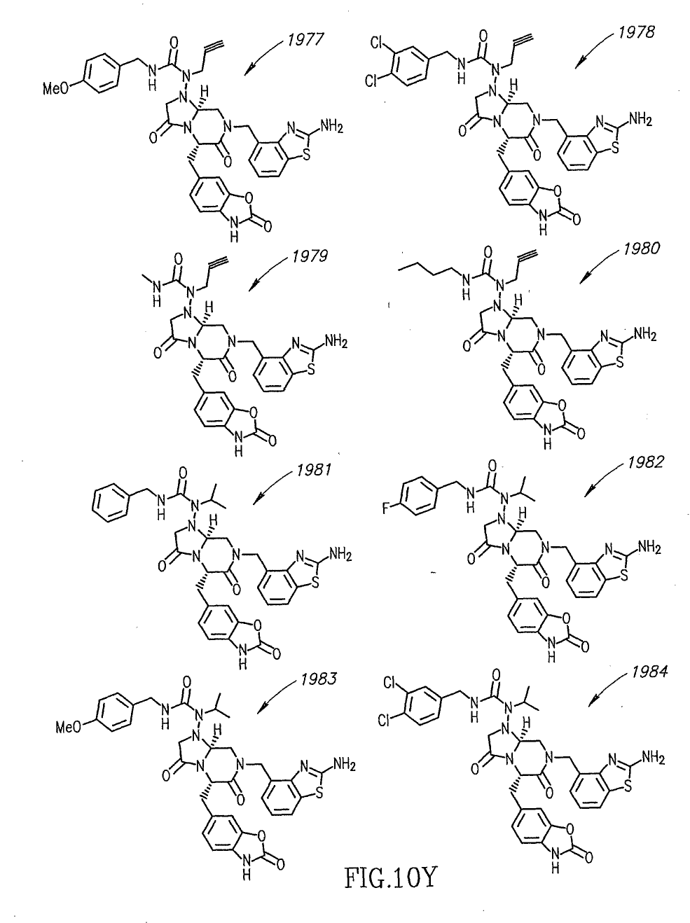

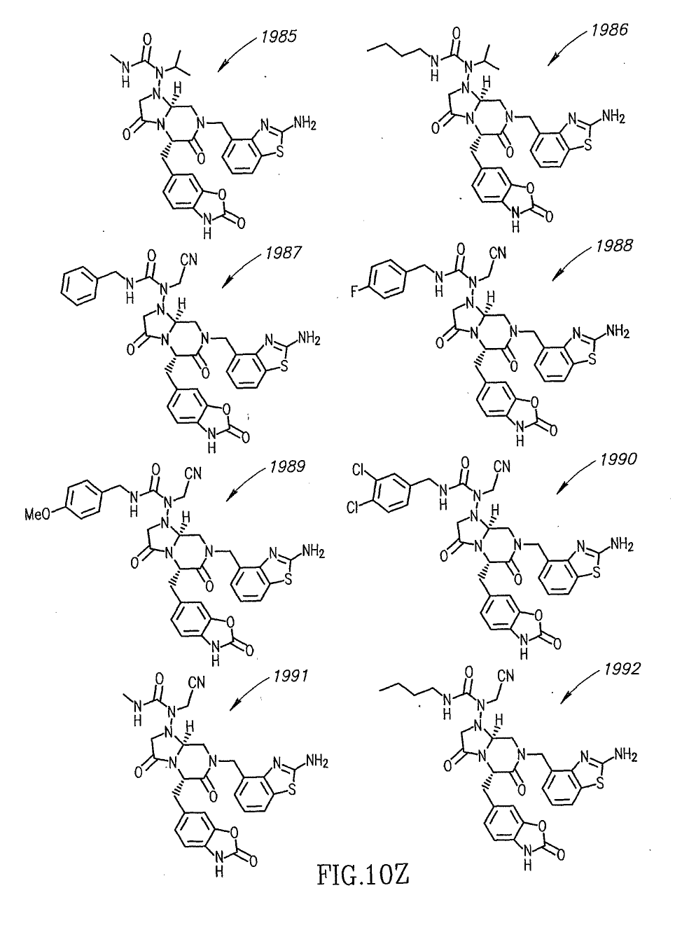

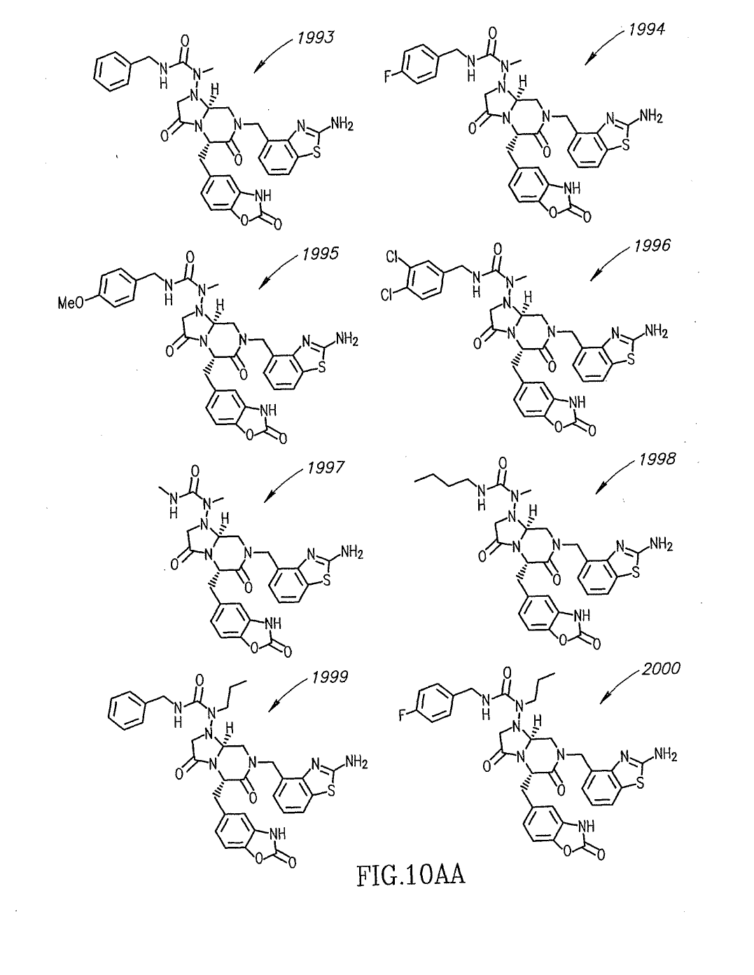

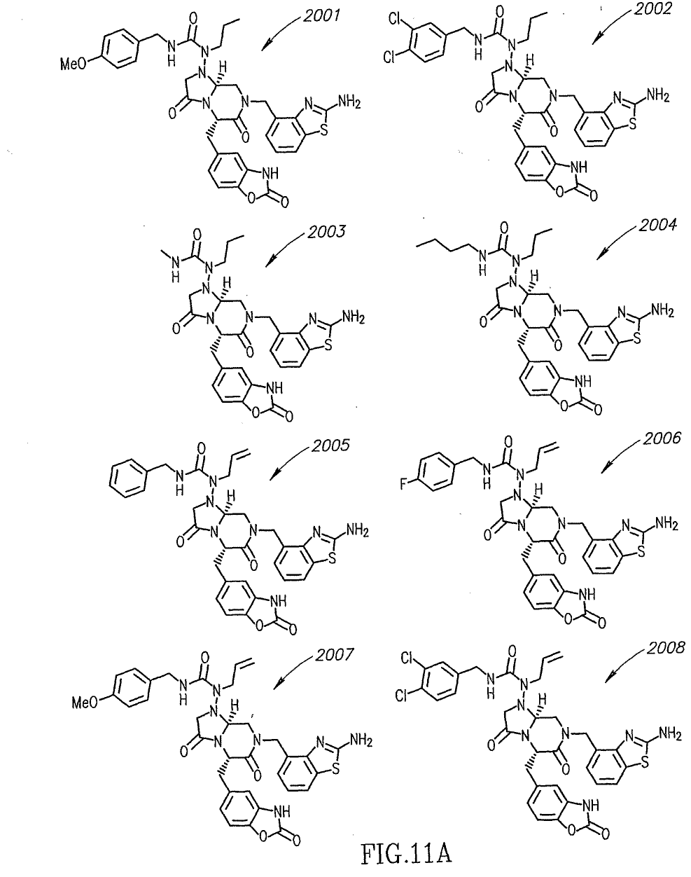

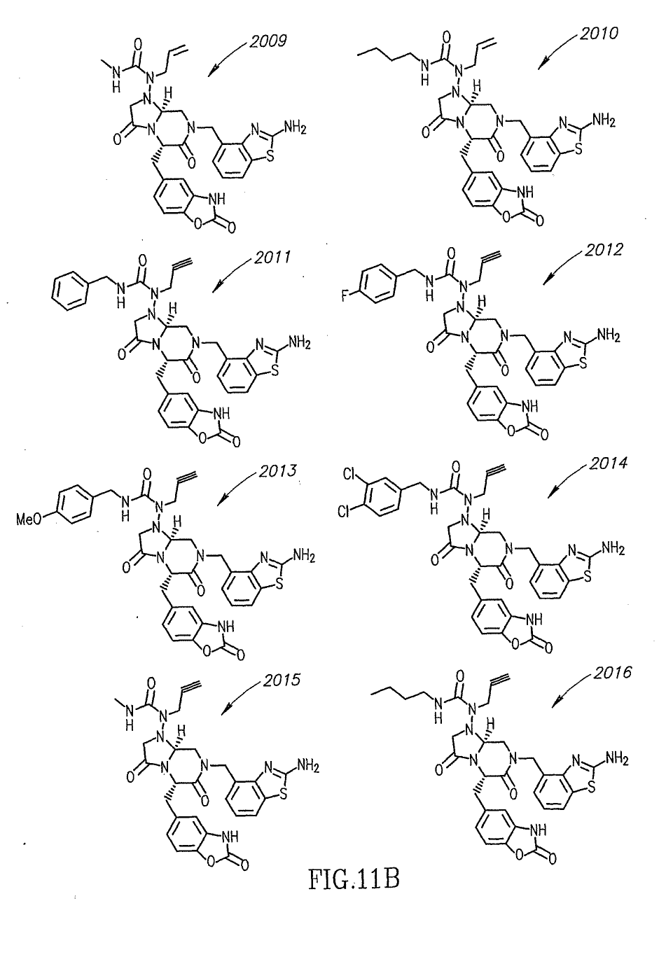

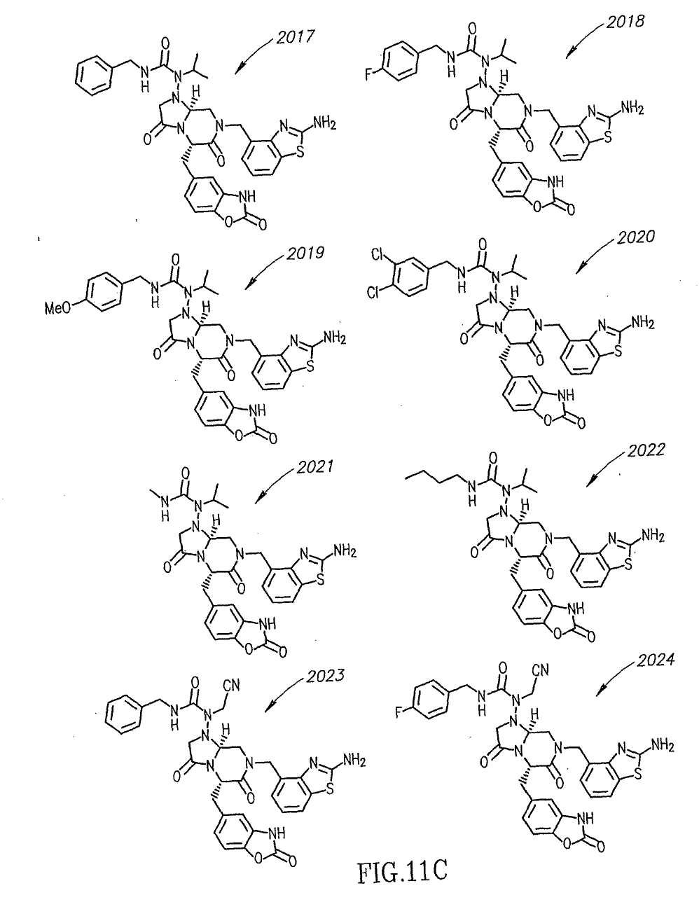

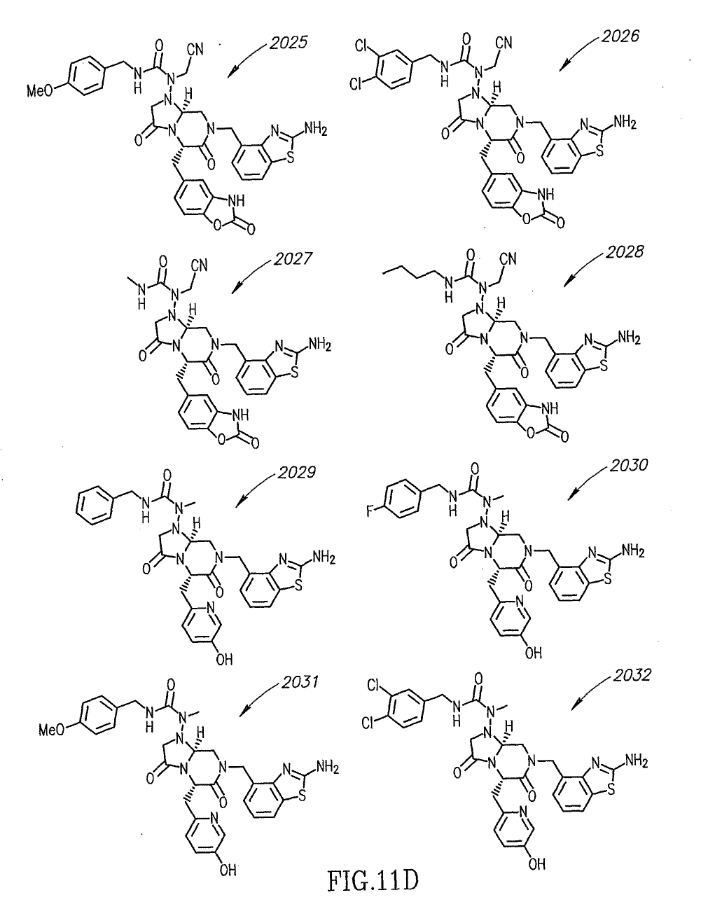

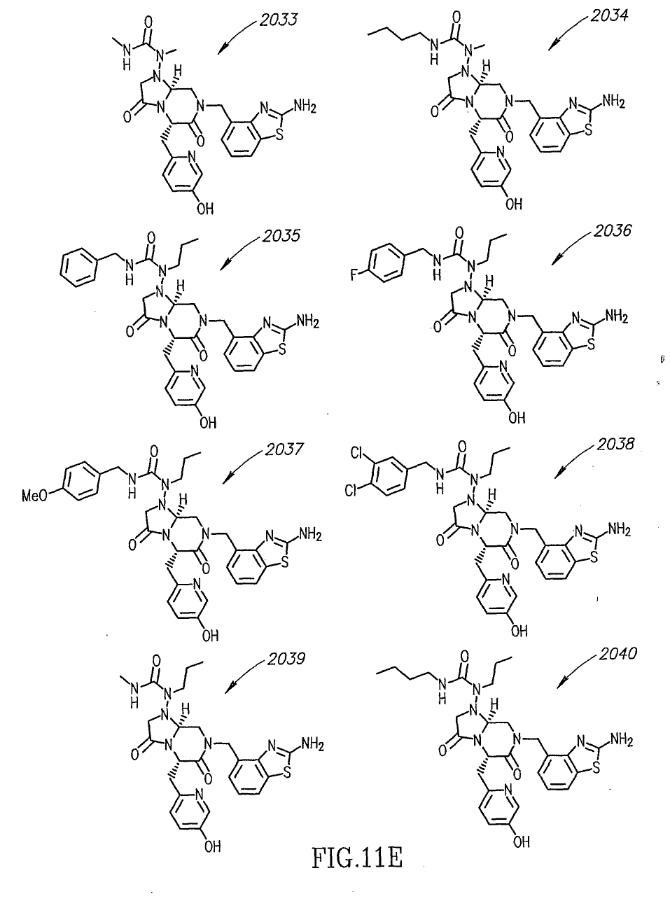

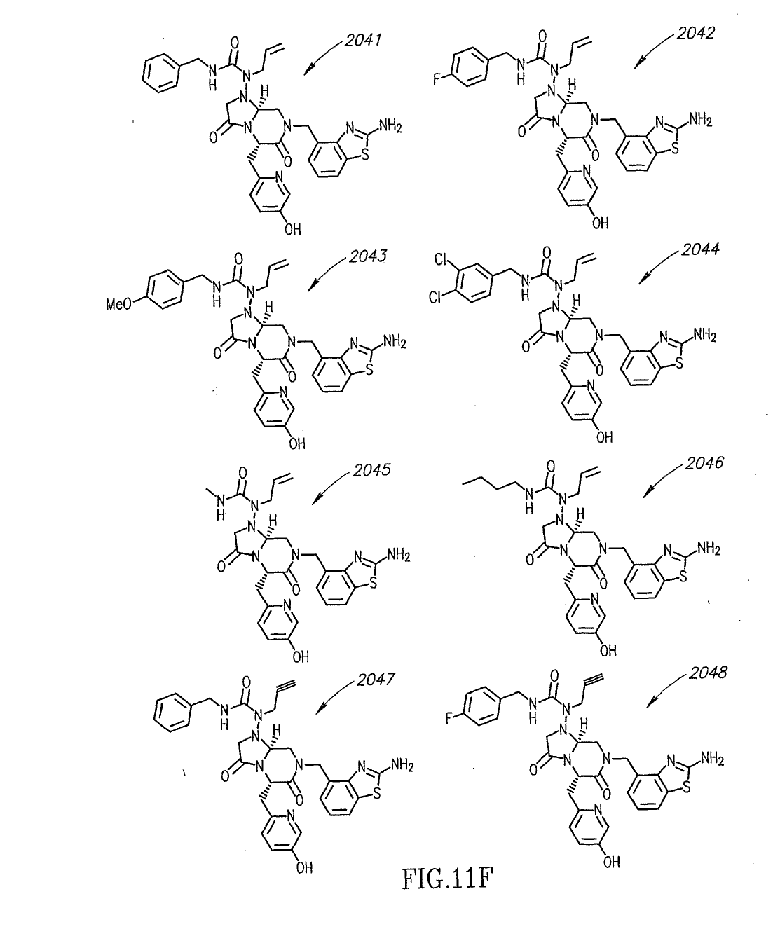

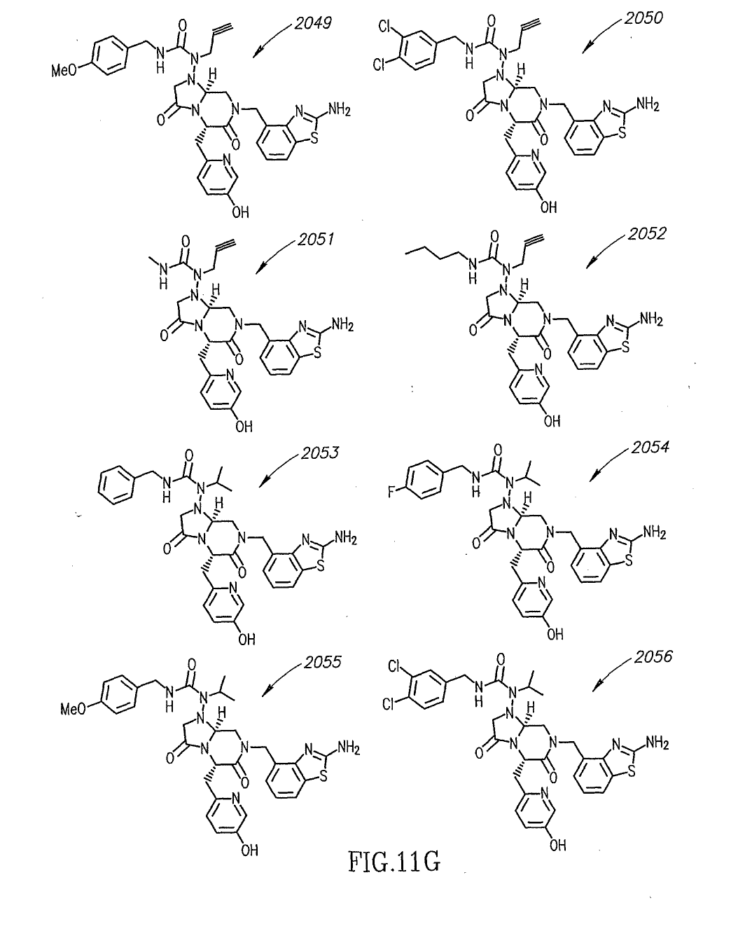

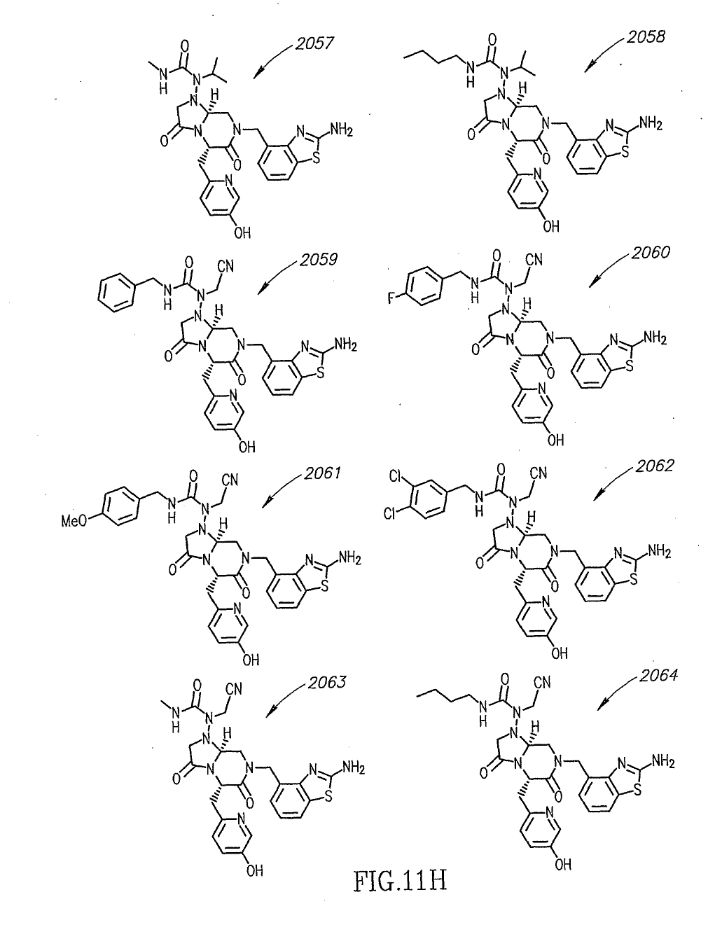

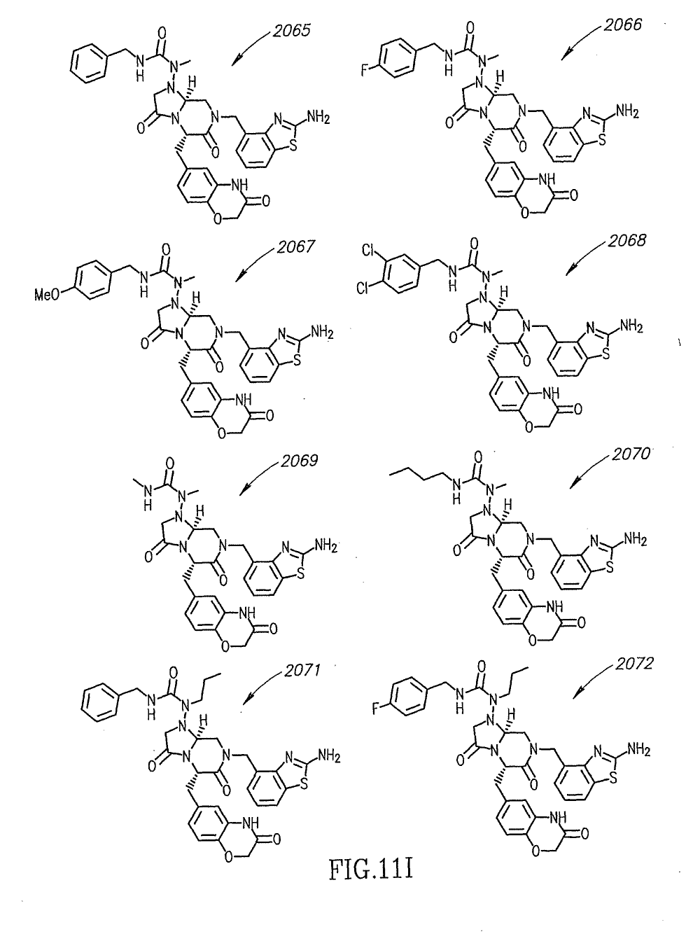

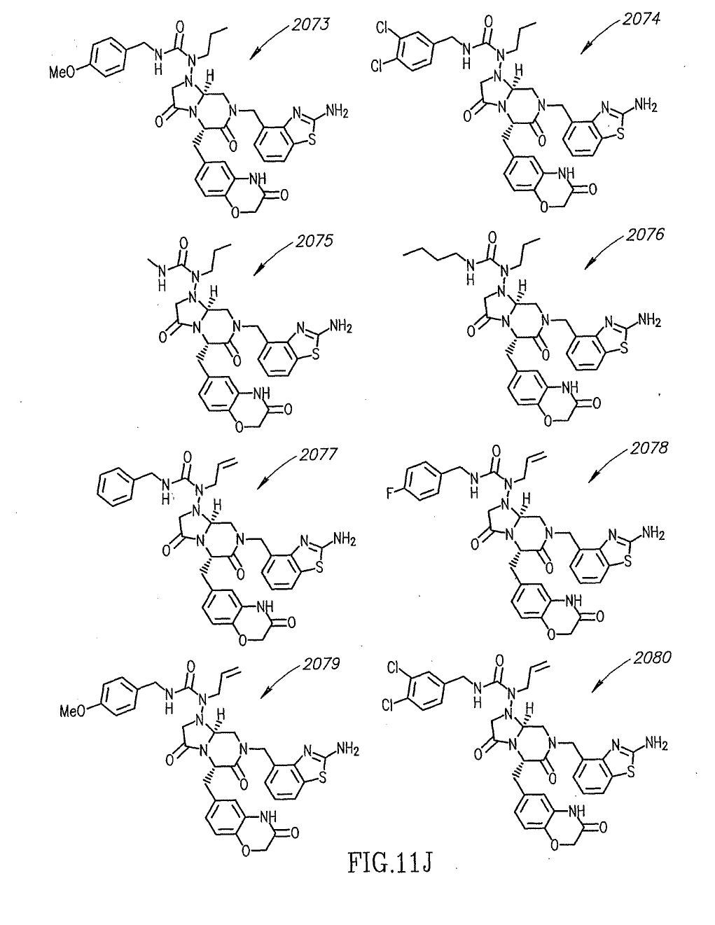

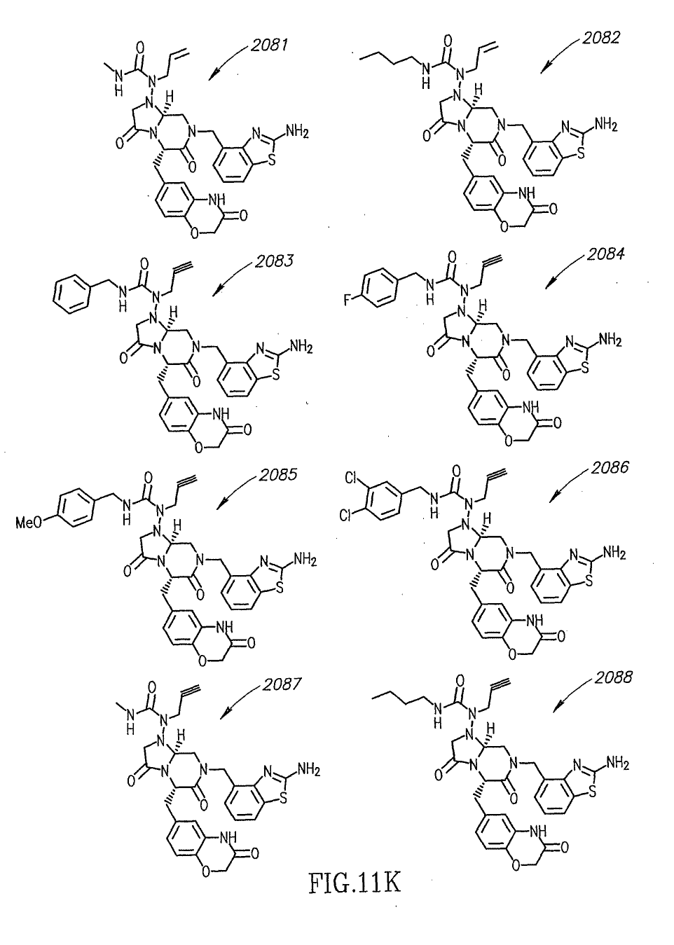

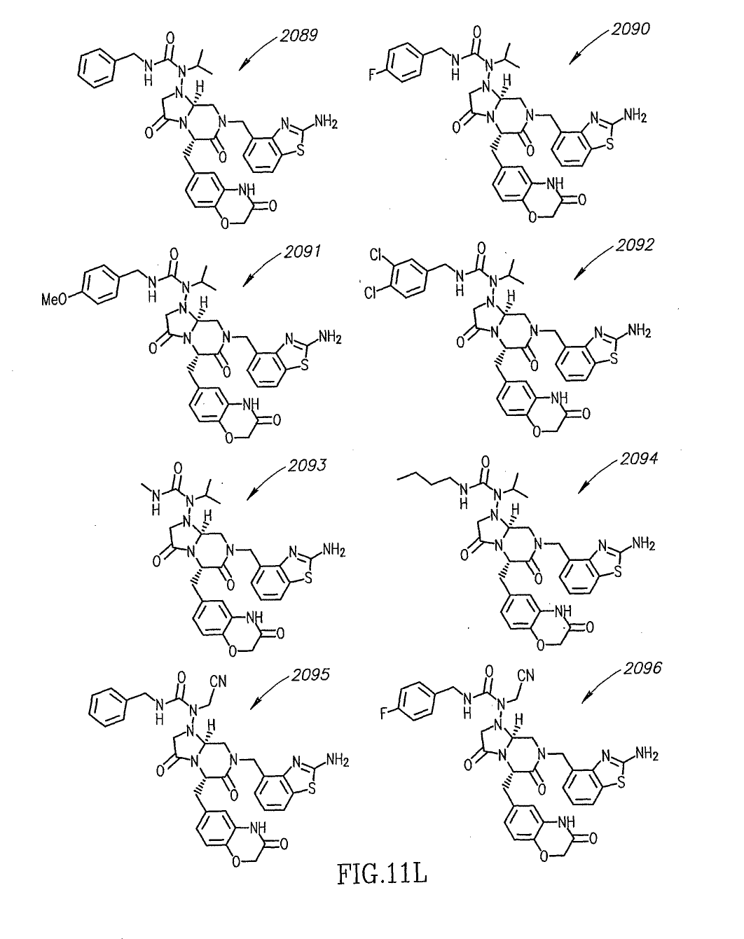

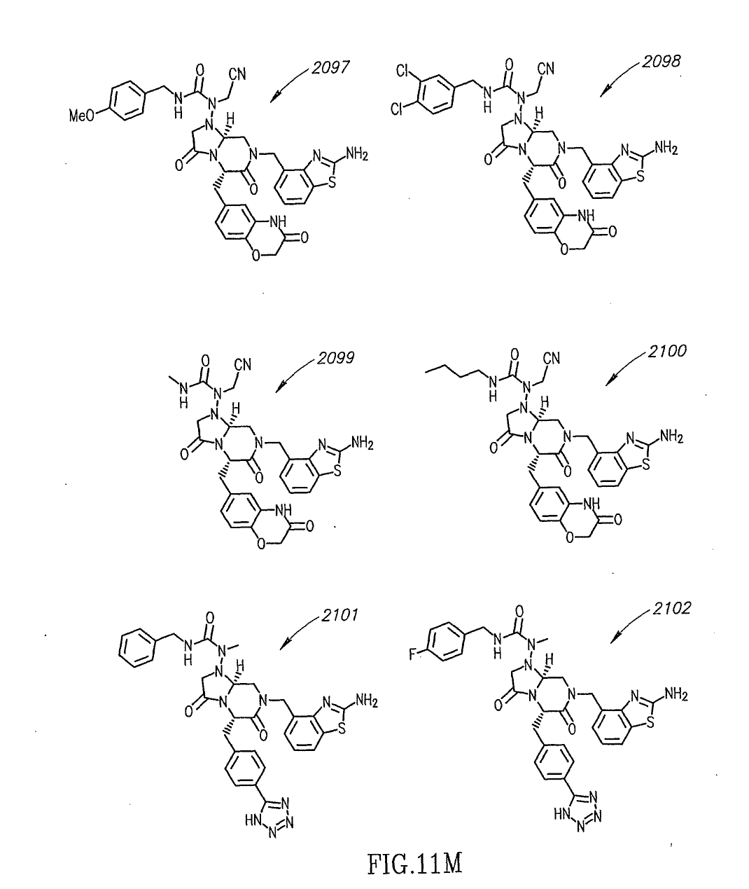

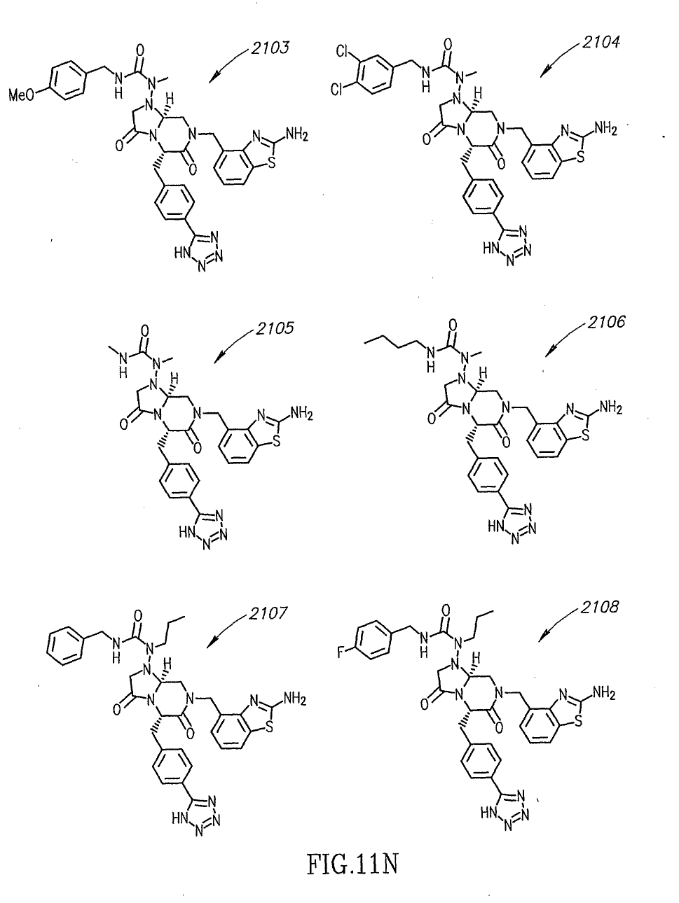

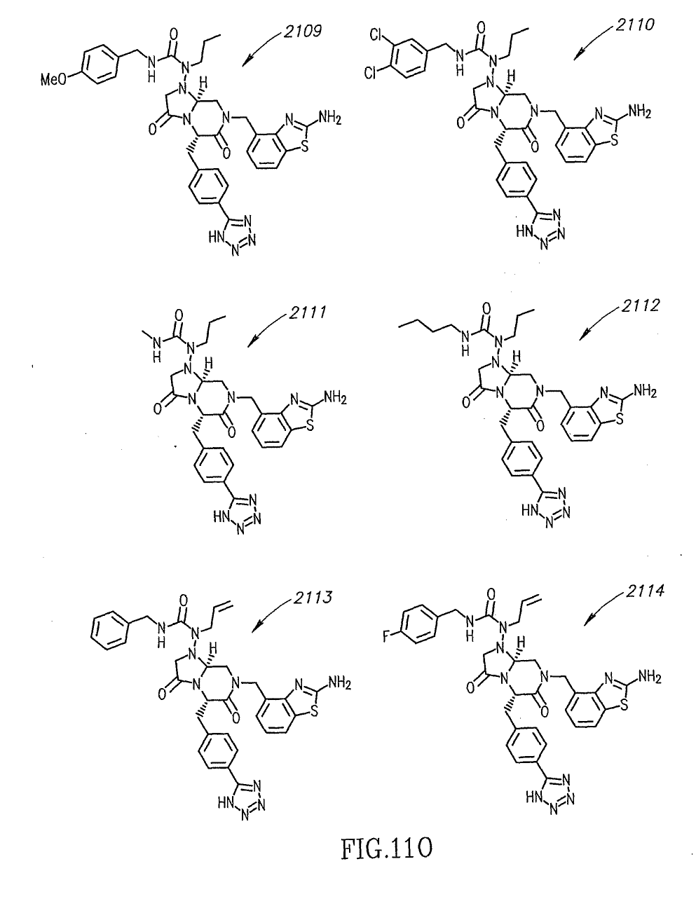

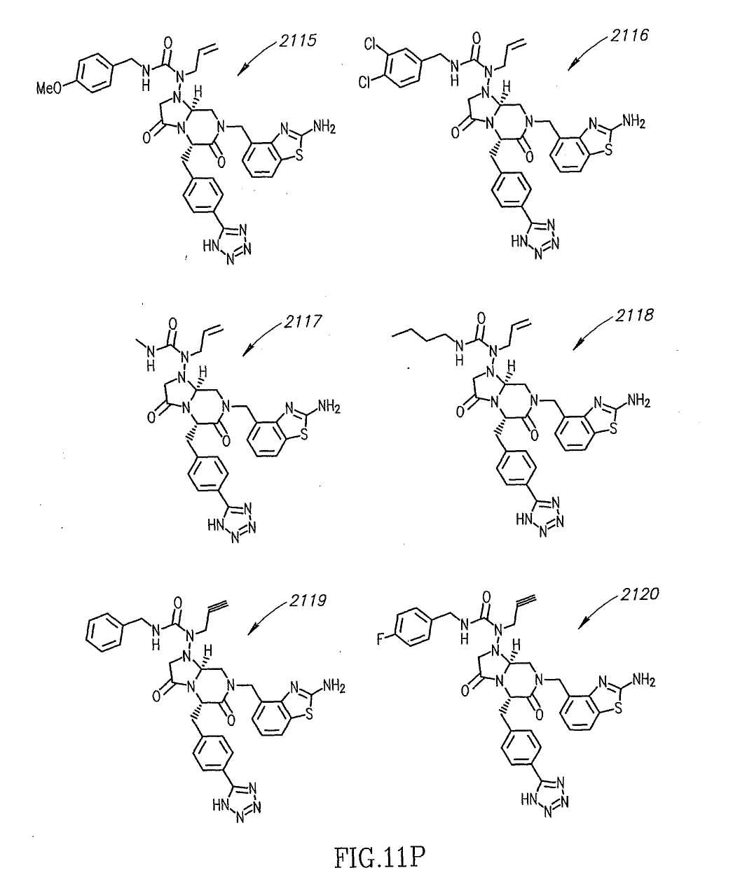

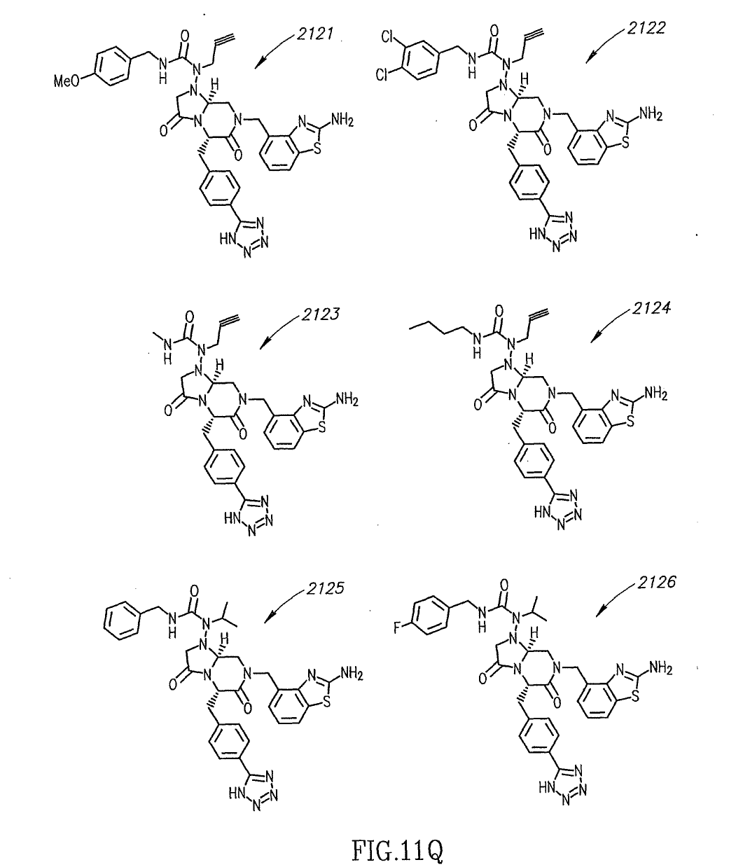

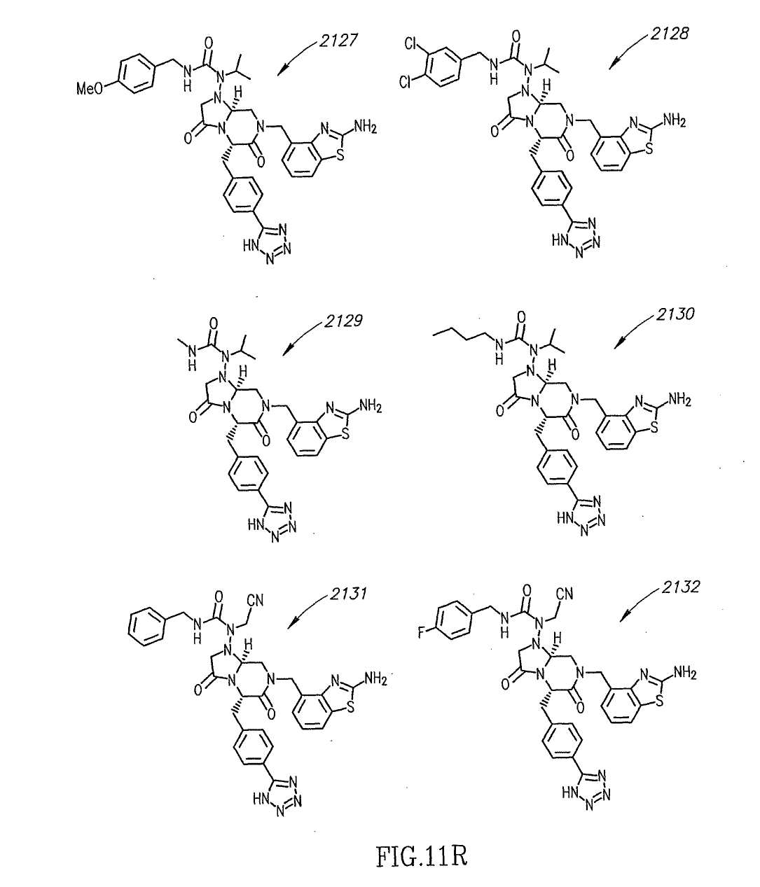

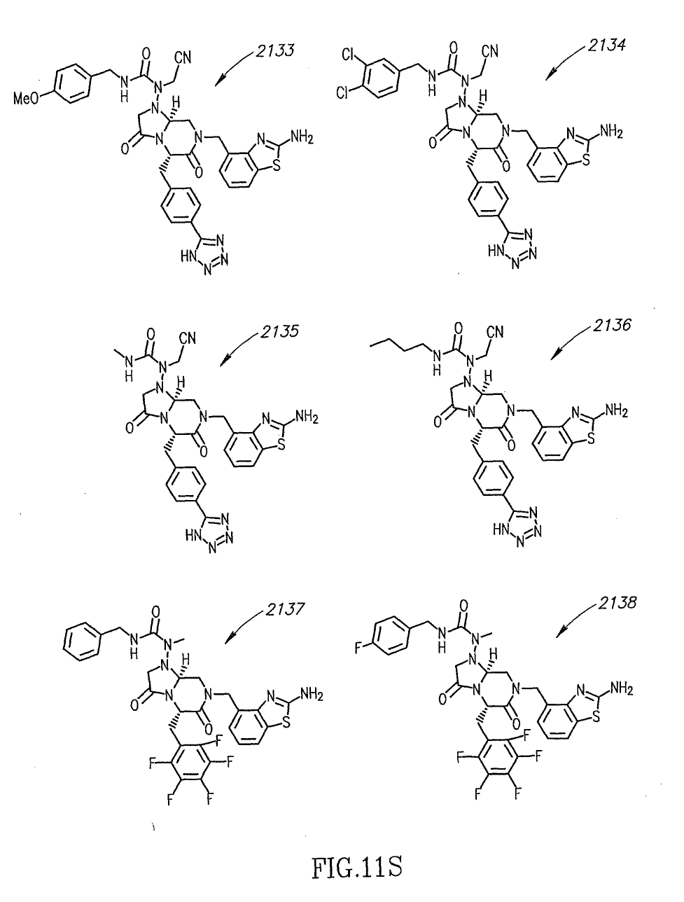

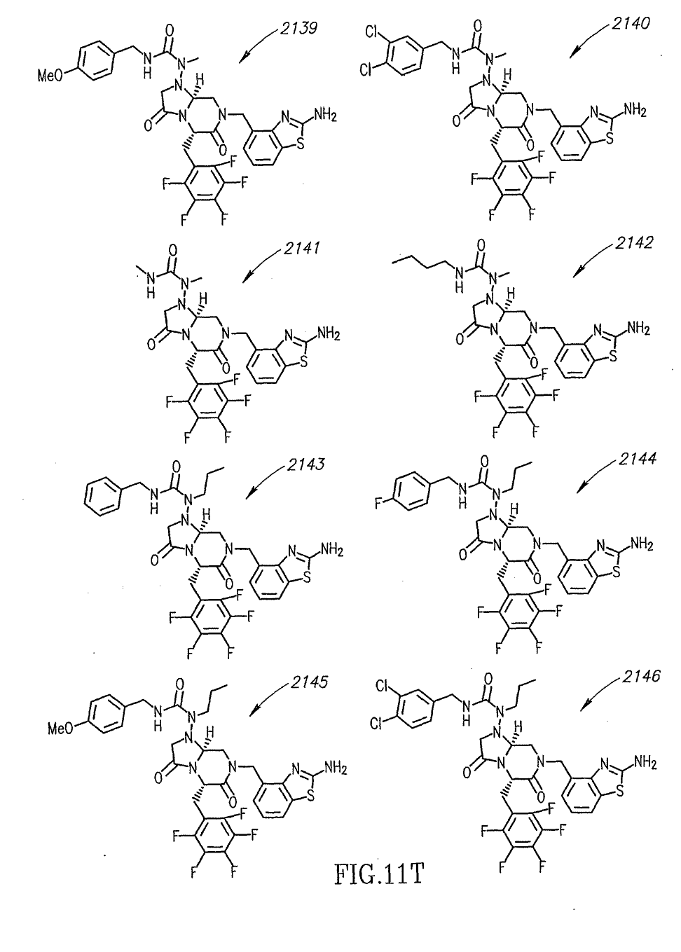

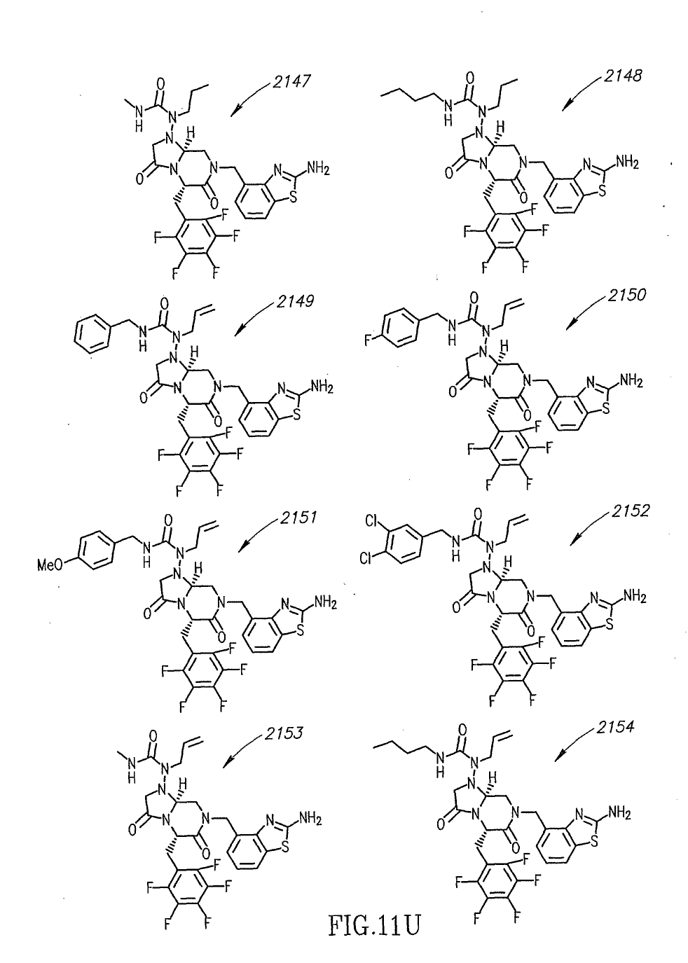

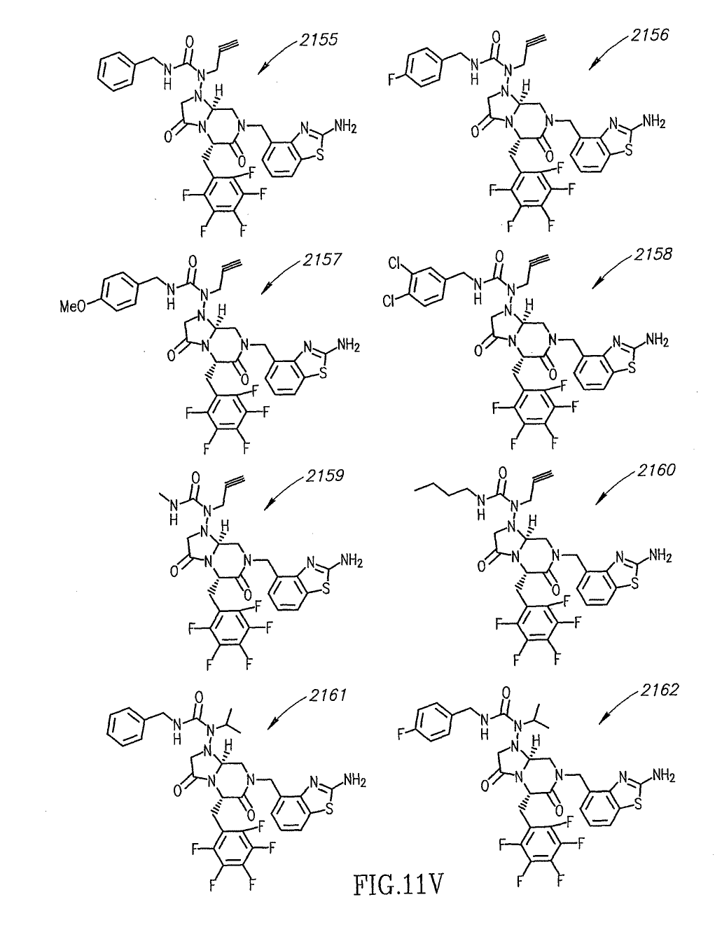

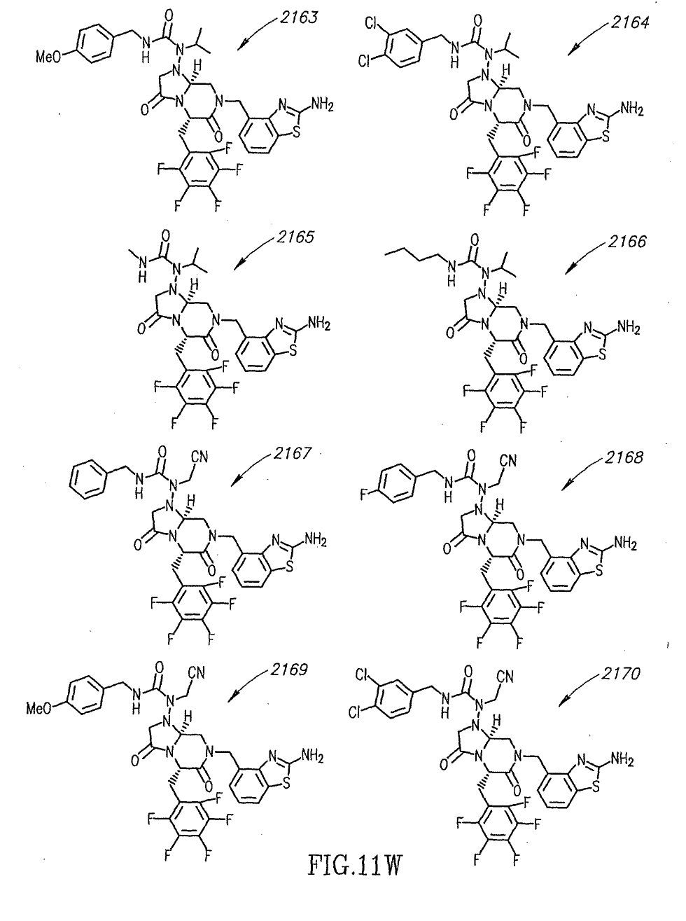

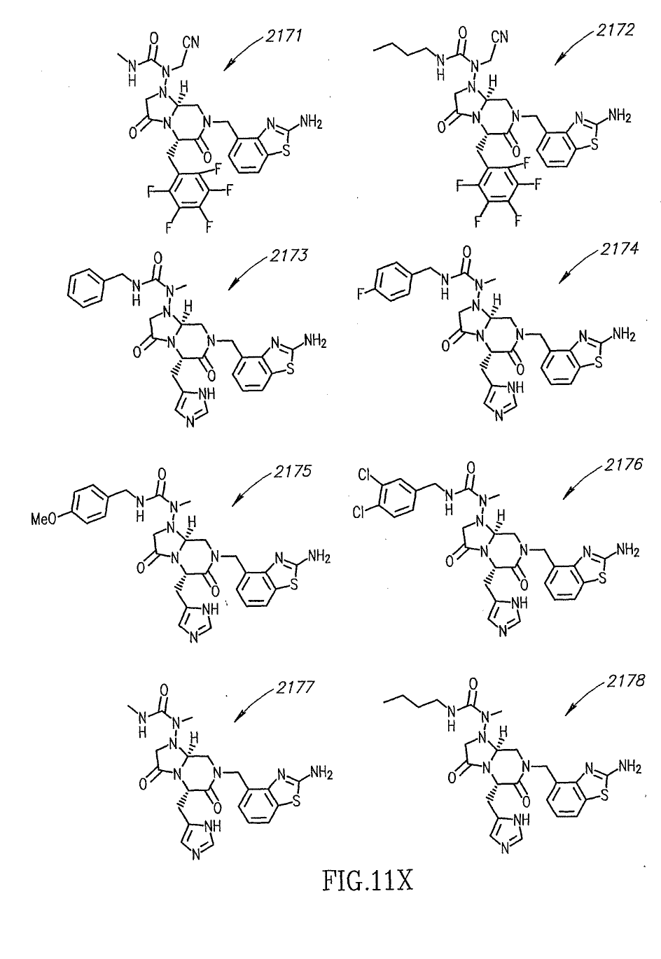

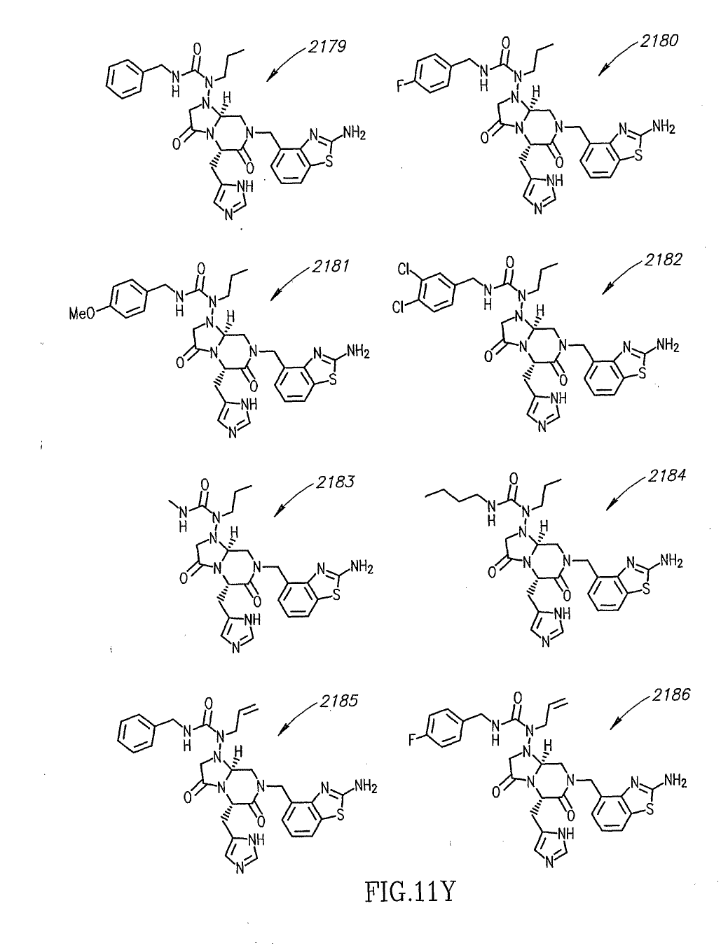

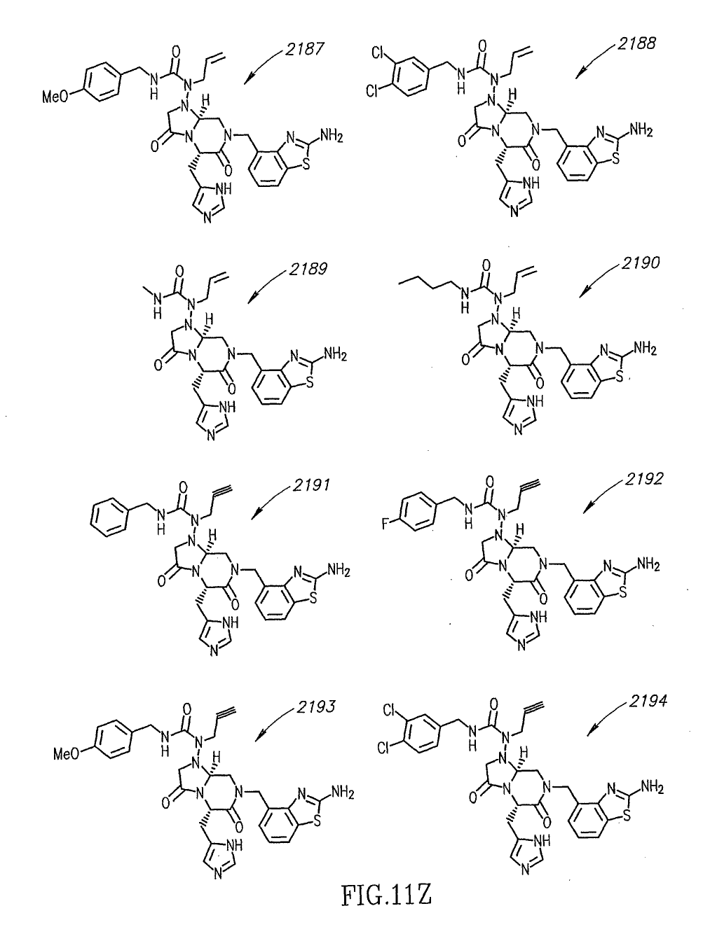

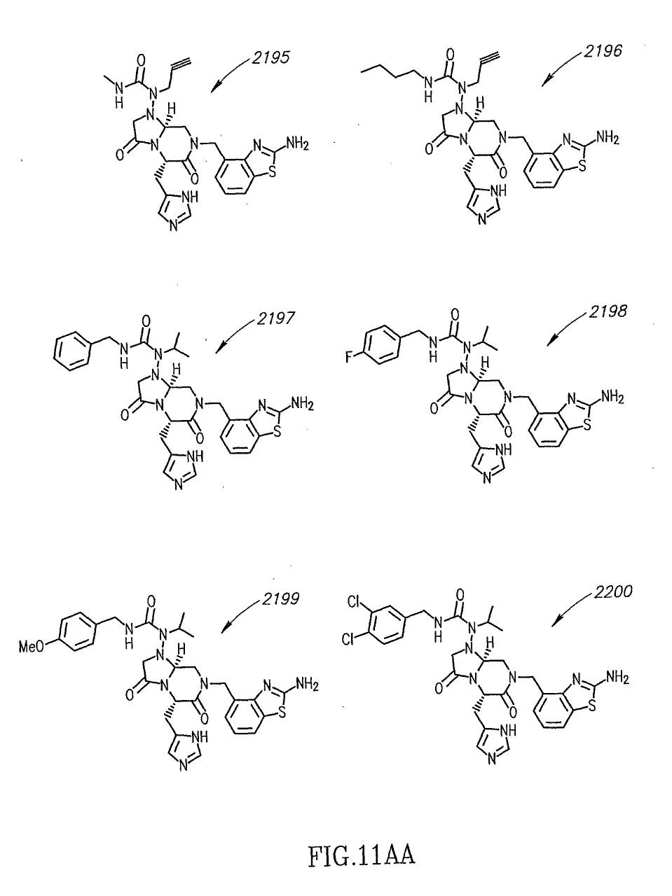

Non-limiting embodiments of these structures are shown as Compounds 1-2217, FIGS. 1-12 and 26.

In another aspect of this invention, libraries containing α-helix mimetic structures of the present invention are disclosed. Once assembled, the libraries of the present invention may be screened to identify individual members having bioactivity. Such screening of the libraries for bioactive members may involve; for example, evaluating the binding activity of the members of the library or evaluating the effect the library members have on a functional assay. Screening is normally accomplished by contacting the library members (or a subset of library members) with a target of interest, such as, for example, an antibody, enzyme, receptor or cell line. Library members, which are capable of interacting with the target of interest, are referred to herein as “bioactive library members” or “bioactive mimetics”. For example, a bioactive mimetic may be a library member which is capable of binding to an antibody or receptor, which is capable of inhibiting an enzyme, or which is capable of eliciting or antagonizing a functional response associated, for example, with a cell line. In other words, the screening of the libraries of the present invention determines which library members are capable of interacting with one or more biological targets of interest. Furthermore, when interaction does occur, the bioactive mimetic (or mimetics) may then be identified from the library members. The identification of a single (or limited number) of bioactive mimetic(s) from the library yields α-helix mimetic structures which are themselves biologically active, and thus useful as diagnostic, prophylactic or therapeutic agents, and may further be used to significantly advance identification of lead compounds in these fields.

In another aspect of this invention, methods for constructing the libraries are disclosed. Traditional combinatorial chemistry techniques (see, e.g., Gallop et al., J. Med. Chem. 37:1233-1251, 1994) permit a vast number of compounds to be rapidly prepared by the sequential combination of reagents to a basic molecular scaffold. Combinatorial techniques have been used to construct peptide libraries derived from the naturally occurring amino acids. For example, by taking 20 mixtures of 20 suitably protected and different amino acids and coupling each with one of the 20 amino acids, a library of 400 (i.e., 20²) dipeptides is created. Repeating the procedure seven times results in the preparation of a peptide library comprised of about 26 billion (i.e., 20⁸) octapeptides.

Specifically, synthesis of the peptide mimetics of the library of the present invention may be accomplished using known peptide synthesis techniques, for example, the General Scheme of [4,4,0] α-helix Mimetic Library as follows:

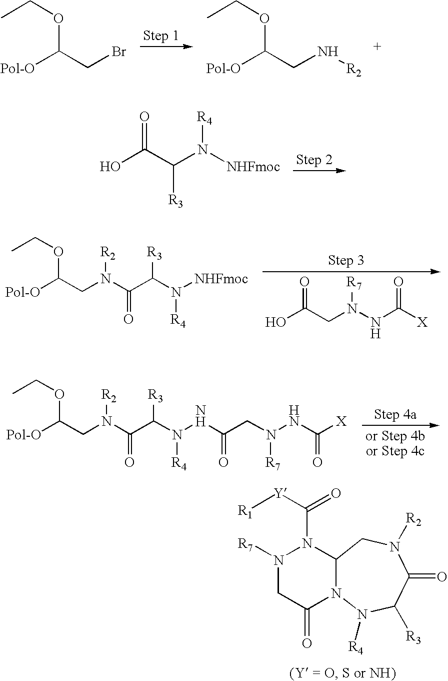

Synthesis of the peptide mimetics of the libraries of the present invention was accomplished using a FlexChem Reactor Block which has 96 well plates by known techniques. In the above scheme ‘Pol’ represents a bromoacetyl resin (Advanced ChemTech) and detailed procedure is illustrated below.

### Step 1

A bromoacetyl resin (37 mg, 0.98 mmol/g) and a solution of R₂-amine in DMSO (1.4 mL) were placed in a Robbins block (FlexChem) having 96 well plates. The reaction mixture was shaken at 60° C. using a rotating oven [Robbins Scientific] for 12 hours. The resin was washed with DMF, MeOH and then DCM

### Step 2

A solution of available Fmoc hydrazine Amino Acids (4 equiv.), PyBop (4 equiv.), HOAt (4 equiv.), and DIEA (12 equiv.) in DMF was added to the resin. After the reaction mixture was shaken for 12 hours at room temperature, the resin was washed with DMF, MeOH and them DCM.

### Step 3

To the resin swollen by DMF before reaction was added 25% piperidine in DMF and the reaction mixture was shaken for 30 min at room temperature. This deprotection step was repeated again and the resin was washed with DMF, Methanol, and then DCM. A solution of hydrazine acid (4 equiv.), HOBt (4 equiv.), and DIC (4 equiv.) in DMF was added to the resin and the reaction mixture was shaken for 12 hours at room temperature. The resin was washed with DMF, MeOH, and then DCM.

### Step 4a (Where Hydrazine Acid is MOC Carbamate)

The resin obtained in Step 3 was treated with formic acid (1.2 mL each well) for 18 hours at room temperature. After the resin was removed by filtration, the filtrate was condensed under a reduced pressure using SpeedVac [SAVANT] to give the product as oil. The product was diluted with 50% water/acetonitrile and then lyophilized after freezing.

### Step 4b (Where Fmoc Hydrazine Acid is Used to Make Urea Through Isocyanate)

To the resin swollen by DMF before reaction was added 25% piperidine in DMF and the reaction mixture was shaken for 30 min at room temperature. This deprotection step was repeated again and the resin was washed with DMF, Methanol, then DCM. To the resin swollen by DCM before reaction was added isocyanate (5 equiv.) in DCM. After the reaction mixture was shaken for 12 hours at room temperature the resin was washed with DMF, MeOH, then DCM. The resin was treated with formic acid (1.2 mL each well) for 18 hours at room temperature. After the resin was removed by filtration, the filtrate was condensed under a reduced pressure using SpeedVac [SAVANT] to give the product as oil. The product was diluted with 50% water/acetonitrile and then lyophilized after freezing.

### Step 4c (Where Fmoc-Hydrazine Acid is Used to Make Urea Through Active Carbamate)

To the resin swollen by DMF before reaction was added 25% piperidine in DMF and the reaction mixture was shaken for 30 min at room temperature. This deprotection step was repeated again and the resin was washed with DMF, MeOH and then DCM. To the resin swollen by DCM before reaction was added p-nitrophenyl chloroformate (5 equiv.) and diisopropyl ethylamine (5 equiv.) in DCM. After the reaction mixture was shaken for 12 hours at room temperature, the resin was washed with DMF, MeOH and then DCM. To the resin was added primary amines in DCM for 12 hours at room temperature and the resin was washed with DMF, MeOH, and then DCM. After reaction the resin was treated with formic acid (1.2 mL each well) for 18 hours at room temperature. After the resin was removed by filtration the filtrate was condensed under a reduced pressure using SpeedVac [SAVANT] to give the product as oil. The product was diluted with 50% water/acetonitrile and then lyophilized after freezing.

To generate these block libraries the key intermediate hydrazine acids were synthesized according to the procedure illustrated in the Examples.

FIG. 13 shows the scaffold of ICG-001 (FIG. 13A) and ASN 06387747 (Asinex) (FIG. 13B). Flexible alignment calculations using MOE (Molecular Operating Environment) revealed that chemical features of ICG-001 were also found in ASN 06387747. A three dimensional alignment of the two molecules is shown in FIG. 13C.

### Administration and Dosage

The inventive compounds may be administered by any means known to one of ordinary skill in the art. For example, the inventive compounds may be administered orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrasternal, intracranial, and intraosseous injection and infusion techniques. The exact administration protocol will vary depending upon various factors including the age, body weight, general health, gender and diet of the patient; the determination of specific administration procedures would be routine to an one of ordinary skill in the art.

Compounds 1-2217 are suitable for treating diseases and pathological conditions including but not limited to interstitial lung disease, human fibrotic lung disease, human kidney disease, polycystic kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Alzheimer's disease; tuberous sclerosis complex; Lymphangioleiomyomatosis; pulmonary hypertension; atherosclerosis; restenosis; ulcerative colitis; rheumatoid arthritis; modulation of hair growth; and graft remodeling.

Certain diseases and pathological conditions can be treated by administering at least one compound having the structure of formula (I), wherein the disease or pathological condition is at least one selected from the group consisting of interstitial lung disease, human fibrotic lung disease, human kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's optihalmopathy, glaucoma, scarring, skin lesions, diabetic retinopathy, scleroderma; Lymphangioleiomyomatosis; pulmonary hypertension; atherosclerosis; and graft remodeling.

The inventive compounds may be administered by a single dose, multiple discrete doses or continuous intrusion. Pump means, particularly subcutaneous pump means, are useful for continuous infusion.

Dose levels on the order of about 0.001 mg/kg/d to about 100 mg/kg/d of an inventive compound are useful for the inventive methods. In one embodiment, the dose level is about 0.1 mg/kg/d to about 100 mg/kg/d. In another embodiment, the dose level is about 1 mg/kg/d to about 10 mg/kg/d. The specific dose level for any particular patient will vary depending upon various factors, including the activity and the possible toxicity of the specific compound employed; the age, body weight, general health, sex and diet of the patient; the time of administration; the rate of excretion; the drug combination; the severity of the disease; and the form of administration. Typically, in vitro dosage-effect results provide useful guidance on the proper doses for patient administration. Studies in animal models are also helpful. The considerations for determining the proper dose levels are well known in the art and within the skills of an ordinary physician.

Any known administration regimen for regulating the timing and sequence of drug delivery may be used and repeated as necessary to effect treatment in the inventive methods. The regimen may include pretreatment and/or co-administration with additional therapeutic agent(s).

The inventive compounds can be administered alone or in combination with one or more additional therapeutic agent(s) for simultaneous, separate, or sequential use. Examples of an additional therapeutic agent include, without limitation, compounds of this invention; steroids (e.g., hydrocortisones such as methylprednisolone); anti-inflammatory or anti-immune drug, such as methotrexate, azathioprine, cyclophosphamide or cyclosporin A; interferon-β; antibodies, such as anti-CD4 antibodies; chemotherapeutic agents; immunotherapeutic compositions; electromagnetic radiosensitizers; and morphine. The inventive compounds may be co-administered with one or more additional therapeutic agent(s) either (i) together in a single formulation, or (ii) separately in individual formulations designed for optimal release rates of their respective active agent.

### Pharmaceutical Compositions

This invention further provides a pharmaceutical composition comprising: (i) an effective amount of at least one compound as disclosed herein; and (i) a pharmaceutically acceptable carrier.

The inventive pharmaceutical composition may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and additional therapeutic agent(s).

The inventive pharmaceutical composition may be formulated into solid or liquid form for the following: (1) oral administration as, for example, a drench (aqueous or non-aqueous solution or suspension), tablet (for example, targeted for buccal, sublingual or systemic absorption), bolus, powder, granule, paste for application to the tongue, hard gelatin capsule, soft gelatin capsule, mouth spray, emulsion and microemulsion; (2) parenteral administration by, for example, subcutaneous, intramuscular, intravenous or epidural injection as, for example, a sterile solution, suspension or sustained-release formulation; (3) topical application as, for example, a cream, ointment, or controlled-release patch or spray applied to the skin; (4) intravaginal or intrarectal administration as, for example, a pessary, cream or foam; (5) sublingual administration; (6) ocular administration; (7) transdermal administration; or (8) nasal administration.

It should be understood that the examples and embodiments described herein are for illustrative purposes only and that various modifications or changes in light thereof will be suggested to persons skilled in the art and are to be included within the spirit and purview of this application.

### Example 1

### Intermediate Synthesis

### Synthesis of 2-Boc-amino-benzothiazoleyl-4-methylamine

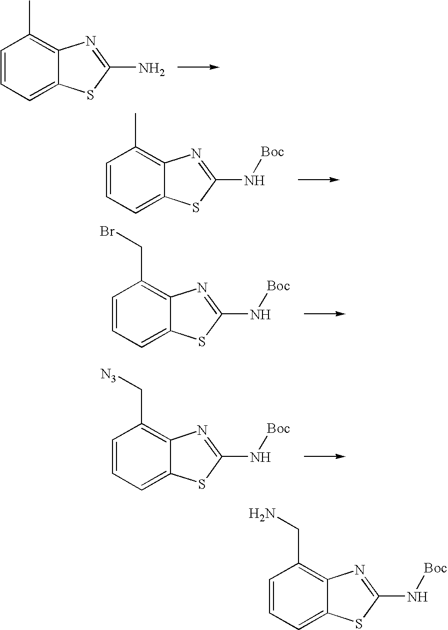

### Step-1 (2-Boc-amino-4-methyl benzothiazole)

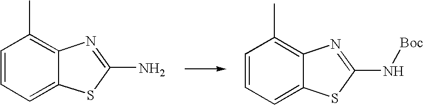

A solution of 2-Amino-4-methyl benzothiazole (25.0 g, 152 mmol) in 456 mL of dry THF was treated with Et₃N (42 mL, 300 mmol), (Boc)₂O (40.0 g, 183 mmol) and DMAP (3.7 g, 30 mmol) at 20° C. and stirred at 30° C. for 12 h. The resulting solution was concentrated hi vacuo, diluted with EtOAc (200 mL) and filtered through a glass filter (Celite) washing with EtOAc (200 mL). The filtrate was washed with NaHCO₃ (saturated aqueous solution, 100 mL) and NaCl (saturated aqueous solution, 100 mL), dried over MgSO₄ and concentrated in vacuo. The residue was filtered through a silica gel plug (flash column chromatography) eluting with toluene:Et₂O=15:1 to 8:1 to afford 2-Boc-amino-4-methyl benzothiazole as a colorless oil (41.4 g, quant.) R_{f}=0.48 (toluene:Et₂O=10:1); ¹H NMR (400 MHz, CDCl₃) δ 9.75 (1H, br s), 7.61 (1H, d, J=7.8 Hz), 7.19 (3H, m), 2.64 (3H, s), 1.47 (9H, s).

### Step-2 (2-Boc-amino-4-bromomethyl benzothiazole)

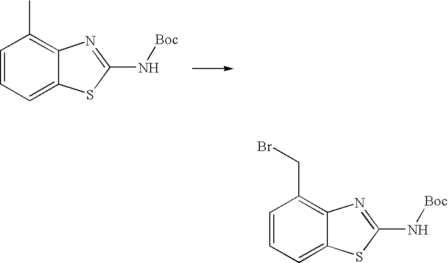

A solution of 2-Boc-aminomethyl benzothiazole (152 mmol) in 456 mL of dry CCl4 was treated with NBS (27.1 g, 152 mmol) and AIBN (3.2 g, 20 mmol) at 20° C. and stirred at 80° C. for 3.5 h. The mixture was retreated with NBS (7.2 g, 41 mmol) and AIBN (0.84 g, 5.1 mmol) at 20° C. and stirred at 80° C. for 11 hr. The resulting mixture was cooled to 20° C. and filtered through a glass filter (Celite) washing with Et₂O (200 mL). The filtrate was concentrated in vacuo. The residue was filtered through a silica gel column (flash column chromatography) eluting with toluene:Et₂O=20:1 to 10:1 to afford 2-BocNH-4-bromomethyl benzothiazole (46.7 g, 136 mmol, 90%) as a yellowish oil. R_{f}=0.51 (toluene:Et₂O=5:1); ¹H NMR (400 Mz, CDCl₃) δ 8.27 (1H, br s), 7.72 (1H, d, J=8.2 Hz), 7.43 (1H, d, J=7.2 Hz), 7.24 (1H, dd, J=8.2, 7.2 Hz), 4.91 (2H, s), 1.56 (9H, s).

### Step-3 (2-Boc-amino-4-azidomethyl benzothiazole)

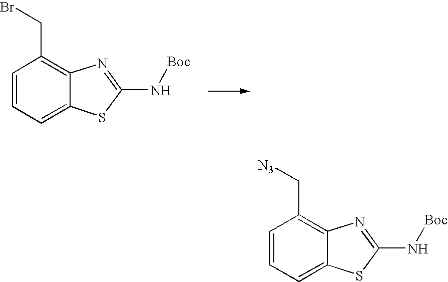

A solution of 2-Boc-amino-4-bromomethyl benzothiazole (46.7 g, 136 mmol) in 205 mL of dry DMF was treated with NaN₃ (8.80 g, 136 mmol) at 15° C. and stirred at 20° C. for 45 min. The resulting mixture was diluted with Et₂O (400 mL), quenched by addition of NaCl (1 g in 150 mL of H₂O) at 0° C. The solution was extracted with Et₂O (100 mL). The organic phase was washed with NaCl (2 g in 100 mL of H₂O) twice, dried over MgSO₄ and concentrated in vacuo. The residue was filtered through a silica gel plug (flash column chromatography) eluting with toluene:Et₂O=100:0 to 10:1 to afford 2-Boc-amino-4-azidomethyl benzothiazole (33.2 g, 109 mmol, 80%) as a colorless oil. R_{f}=0.48 (toluene:Et₂O=10:1); ¹H NMR (400 MHz, CDCl₃) δ 7.75 (1H, d, J=8.2 Hz), 7.37 (1H, d, J=7.2 Hz), 7.27 (1H, m), 4.74 (2H, s), 1.52 (9H, s); ¹³C NMR (99.5 MHz, CDCl₃) δ 159.8, 151.9, 147.6, 132.5, 127.6, 125.8, 123.5, 121.3, 83.4, 51.4, 28.1.

### Step-4 (2-Boc-amino-benzothiazoleyl-4-methylamine)

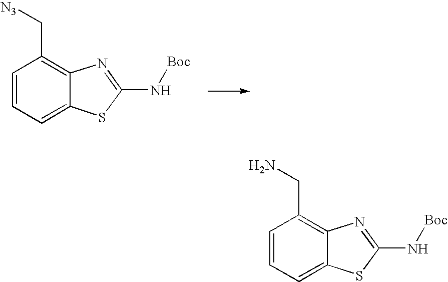

A solution of 2-Boc-amino-4-azidomethyl benzothiazole (11.6 g, 38.0 mmol) in 183 mL of MeOH was treated with Pd(OH)₂ (20% on carbon, 2.9 g), placed under an atmosphere of hydrogen and stirred at 20° C. for 1.5 hr. The resulting mixture was filtered through Celite washing with MeOH:NH₄OH (100:3, 100 mL) and concentrated in vacuo. The obtained yellowish solid was triturated with toluene (35 mL) and filtered to afford 2-Boc-amino-benzothiazolyl-4-methylamine (6.90 g, 24.7 mmol, 65%) as a colorless powder. R_{f}=0.32 (CHCl₃:MeOH:NH₄OH=100:25:1); ¹H NMR (400, CDCl₃) δ 7.67 (1H, d, J=7.7 Hz), 7.25-7.15 (2H, m), 4.85 (2H, br s), 1.58 (9H, s); ¹³C NMR (99.5 MHz, CDCl₃) δ 160.0, 152.8, 148.0, 134.5, 132.7, 124.4, 123.1, 120.0, 82.4, 44.3, 28.3; LC/MS [ESI+] (m/z) 280.2 (M+1)⁺.

### Synthesis of Benzothiazolyl-4-methylamine

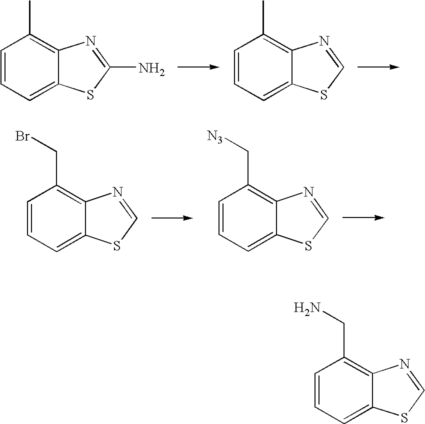

### Step-1 (4-Methyl benzothiazole)

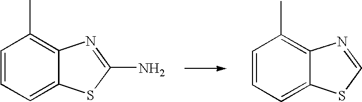

A solution of 2-amino-4-methylbenzothiazole (24.5 g, 149 mmol) in 745 mL of 1,4-dioxane was treated with isoamylnitrite (40.0 mL, 300 mmol) at 20° C. and stirred at 70° C. for 0.5 hr. After the nitrogen evolution had subsided, the mixture was stirred at the same temperature for 1.5 h and concentrated in vacuo. The residue was submitted to silica gel column chromatography with hexane:Et₂O=3:1 to 2:1 as eluate to afford 4-methyl benzothiazole as a yellowish oil. (16.0 g, 107 mmol, 72%) R_{f}=0.45 (toluene:Et₂₀10:1); ¹H NMR (400 MHz, CDCl₃) δ 8.98 (1H, s), 7.79 (1H, d, J=6.8 Hz), 7.33 (2H, m), 2.80 (3H, s).

### Step-2 (4-Bromomethyl benzothiazole)

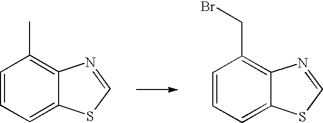

A solution of 4-Methyl benzothiazole (16.0 g, 107 mmol) in 535 mL of CCl4 was treated with NBS (19.0 g, 107 mmol) and AIBN (2.28 g, 13.9 mmol) at 20° C. and stirred at 70° C. for 2.5 h. The resulting mixture was filtered through Celite washing with Et₂O (150 mL) and concentrated in vacuo. The residue was submitted to a silica gel column chromatography with toluene:Et₂O=50:3 to 50:5 as eluate to afford 4-bromomethyl benzothiazole as a yellowish solid. (20.4 g, 89.9 mmol, 84%) R_{f}=0.61 (toluene-Et₂O 10:1); ¹H NMR (400 MHz, CDCl₃) δ 9.07 (1H, s), 7.90 (1H, d, J=7.5 Hz), 7.55 (1H, d, J=7.5 Hz), 7.41 (1H, t, J=7.5 Hz), 5.08 (2H, s); ¹³C NMR (99.5 MHz, CDCl₃) δ 154.1, 151.4, 134.3, 132.6, 127.0, 125.6, 122.3, 29.5.

### Step-3 (4-Azidomethyl benzothiazole)

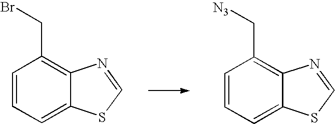

A solution of 4-Bromomethyl benzothiazole (20.4 g, 89.9 mmol) in 272 mL of dry DMF was treated with NaN₃ (7.00 g, 108 mmol) at 20° C. and stirred at the same temperature for 5 min. The resulting mixture was quenched by addition of NaCl (5 g in 150 mL of H₂O) at 0° C., diluted with Et₂O (200 mL) and extracted with Et₂O (200 mL×6). The organic phase was washed with NaCl (2 g in 100 mL of H₂O) twice and brine (100 mL). The resulting solution was dried over MgSO₄ and concentrated in vacuo. The residue was submitted to silica gel column chromatography with toluene:Et₂O=50:3 to 50:5 as eluate to afford 4-azidomethyl benzothiazole as a colorless oil (15.5 g, 81.5 mmol, 91%). R_{f}=0.48 (toluene:Et₂O=10:1); ¹H NMR (400 MHz, CDCl₃) δ 9.03 (1H, s), 7.95 (1H, d, J=7.7 Hz), 7.49 (2H, m), 5.01 (2H, s); ¹³C NMR (99.5 MHz, CDCl₃) δ 154.2, 151.7, 134.3, 130.6, 126.0, 125.7, 122.1, 51.6.

### Step 4 (Benzothiazole-4-methylamine)

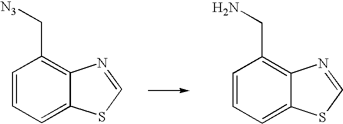

To a solution of 4-Azidomethyl benzothiazole (15.4 g, 81.0 mmol) in 243 mL of MeOH was added Pd(OH)₂ (20% on carbon, 3.1 g) and then hydrogenolysis at 20° C. After 1.5 hr, additional Pd(OH)₂ (20% on carbon, 0.87 g) was added and then hydrogenolysis. After further 1.5 hr, additional Pd(OH)₂ (20% on carbon, 1.27 g) was added and then hydrogenolysis for 1 hr. The resulting mixture was replaced with N₂ and then filtered through Celite washing with MeOH:NH₄OH (25:1, 260 mL) and concentrated in vacuo. The residue was submitted to silica gel column chromatography eluting with CHCl₃:MeOH:NH₄OH (100:0:0 to 20:5:1) followed by trituration with toluene to afford 4-aminomethyl benzothiazole as a white solid (10.5 g, 63.9 mmol, 79%). R_{f}=0.49 (CHCl₃:MeOH:NH₄OH=100:25:1); ¹H NMR (400 MHz, CD₃OD) δ 9.23 (1H, s), 7.97 (1H, d, J=7.7 Hz), 7.46 (2H, m), 4.30 (2H, s); ¹³C NMR (99.5 Mz, CD₃OD) δ 184.2, 180.1, 165.3, 163.5, 154.9, 154.1, 150.1, 72.0; LC/MS [ESI+] (m/z) 165.4 (M+1)⁺.

### Synthesis of 4-Benzyl-3-Boc-2-methylsemicarbazidylacetatic Acid

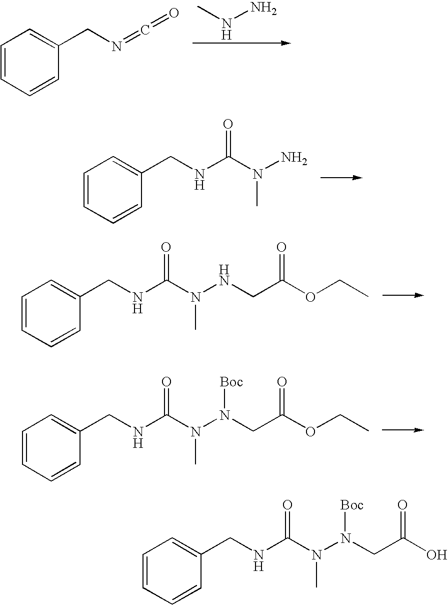

### Step-1 (4-Benzyl-2-methylsemicarbazide)

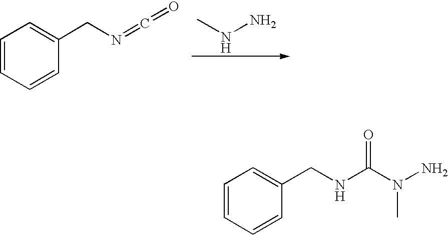

A solution of Benzyl isocyanate (1.85 mL, 15.0 mmol) in 7.5 mL of CHCl₃ was treated with methyl hydrazine (795 μL, 15.0 mmol) at 0° C. and stirred at the same temperature for 2 h. The resulting mixture was dissolved in 1N HCl (200 mL) and the solution was washed with CHCl₃ (50 mL×3). The aqueous phase was adjusted to pH 12 with 2 M NaOHaq and then extracted with CHCl₃ (100 mL×3). The organic phase was dried over Na₂SO₄ and concentrated in vacuo. The residue was recrystallized from hexane-CHCl₃ to afford (1.7 g, 9.5 mmol, 63%) as a colorless crystal. R_{f}=0.44 (CHCl₃:MeOH=9:1); ¹H NMR (400 MHz, DMSO-d6) δ 7.28-7.19 (5H, m), 4.47 (2H, s), 4.20 (2H, d, J=6.3 Hz), 2.96 (3H, s); ¹³C NMR (99.5 MHz, DMSO-d6) δ 159.3, 141.1, 128.1, 127.1, 126.5, 43.1, 37.8; LC/MS [ESI+] (m/z) 180.3 (M+1)⁺.

### Step-2 (Ethyl 4-benzyl-2-methylsemicarbazidylacetate)

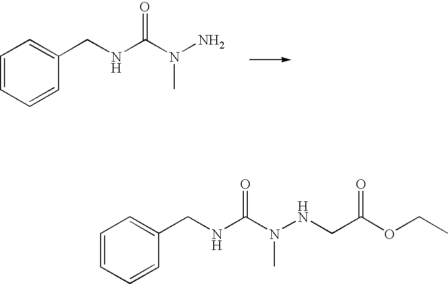

To the solution of 4-Benzyl-2-methylsemicarbazide (5.24 g, 29.2 mmol) in Toluene (58 mL) were added DIPEA (7.63 mL, 43.8 mmol) and Ethyl bromoacetate (4.86 mL, 43.8 mmol) and then stirred at 858 for 24 hr. The reaction mixture was allowed to cool to room temperature followed by dilution with EtOAc (100 mL). The mixture was washed with H₂O (50 mL) and brine (50 mL), dried over Na₂SO₄, filtered and concentrated. The crude was submitted to silica gel (250 g) column chromatography with Hex:EtOAc=1:1 to 1:9 as elute to afford a pale yellow oil (5.75 g, 21.7 mmol, 74%). R_{f}=0.36 (Hex:EtOAc=1:3); ¹H NMR (400 MHz, CDCl₃) δ 7.34-7.21 (5H, m), 6.88 (1H, br s), 4.40 (2H, d, J=5.8 Hz), 4.18 (2H, q, J=7.2 Hz), 3.69 (1H, br t, J=4.8 Hz), 3.58 (2H, d, J=4.8 Hz), 3.08 (3H, s), 1.26 (3H, t, J=7.2 Hz); ¹³C NMR (99.5 MHz, CDCl₃) δ 170.8, 159.3, 139.9, 128.6, 127.6, 127.1, 61.4, 50.1, 44.4, 33.1, 14.2; LC/MS [ESI+] (m/z) 266.3 (M+1)⁺.

### Step-3 (Ethyl 4-benzyl-3-Boc-2-methylsemicarbazidylacetate)

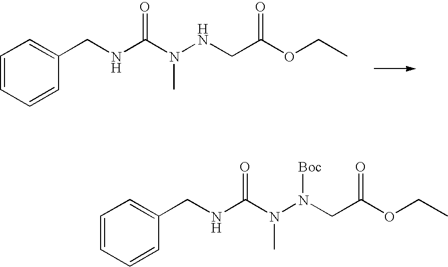

To the solution of Ethyl 4-benzyl-2-methylsemicarbazidylacetate (5.70 g, 21.5 mmol) in CH₂Cl₂ (43 mL) were added DIPEA (7.5 mL, 43 mmol), DMAP (1.1 g, 8.6 mmol) and (BOC)₂O (9.4 g, 43 mmol) and then stirred for 1 hr at room temperature. The reaction mixture was concentrated and then submitted to SiO₂ (250 g) column chromatography with Hex:EtOAc=7:1 to 1:2 as eluate to afford product (2.58 g, 7.06 mmol, 33%) as a pale yellow oil, and starting material (2.80 g, 10.6 mmol, 49%) was recovered. R_{f}=0.76 (Hex:EtOAc=1:3); ¹H NMR (400MHz, CDCl₃) δ 7.54 (1H, br s), 7.33-7.20 (5H, m), 4.59-4.46 (2H, m), 4.27-4.19 (4H, m), 3.72 (1H, br d, J=17 Hz), 3.03 (3H, br s), 1.39 (9H, s), 1.26 (3H, t, J=7.2 Hz); ¹³C NMR (99.5 MHz, CDCl₃) δ 170.7, 158.3, 139.8, 128.3, 127.6, 126.9, 82.7, 62.0, 51.6, 44.3, 34.4, 28.0, 14.1; LC/MS [ESI+] (m/z) 366.3 (M+1)⁺.

### Step-4 (4-Benzyl-3-Boc-2-methylsemicarbazidylacetatic acid)

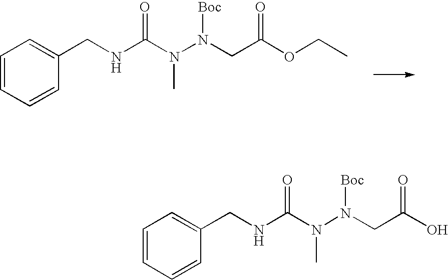

To the solution of Ethyl 4-benzyl-3-Boc-2-methylsemicarbazidylacetate (2.30 g, 6.29 mmol) in THF/MeOH/H₂O (2/3/1, 24 mL) was added LiOH H₂O (528 mg, 12.6 mmol) at 0δ. After stirred for 1 hr at room temperature, the reaction mixture was diluted with EtOAc (40 mL) at 0δ. The mixture was acidified with 1N HCl and then extracted with EtOAc. The combined extracts were washed with H₂O (30 mL) and brine (30 mL), dried over Na₂SO₄, added Et₃N (2 mL), filtered and concentrated. The crude was submitted to SiO₂ column chromatography with CHCl₃:MeOH=100:0 to 85:15 as eluant to afford a pale yellow sticky oil 4-Benzyl-3-Boc-2-methylsemicarbazidylacetatic acidδEt₃N salt (1.99 g, 4.56 mmol, 72%); ¹H NMR (400 MHz, CDCl₃) δ 8.45 (1H, br s), 7.32-7.18 (5H, m), 4.58-4.22 (3H, m), 3.71-3.57 (1H, m), 3.08 and 3.01 (3H, br s), 2.82 (2.4H, q, J=7.3 Hz, Et₃N), 1.40 (9H, br s), 1.08 (3.6H, t, J=7.3 Hz, Et₃N); ¹³C NMR (99.5 MHz, CDCl₃) δ 174.2, 159.2, 154.1, 140.1, 128.2, 127.4, 12.7, 81.8, 52.2, 45.1 (Et₃N), 44.1, 34.5, 28.1, 8.3 (Et₃N); LC/MS [ESI+] (m/z) 338.3 (M+1)⁺.

### Synthesis of 4-Benzyl-3-Boc-2-allylsemicarbazidylacetatic Acid

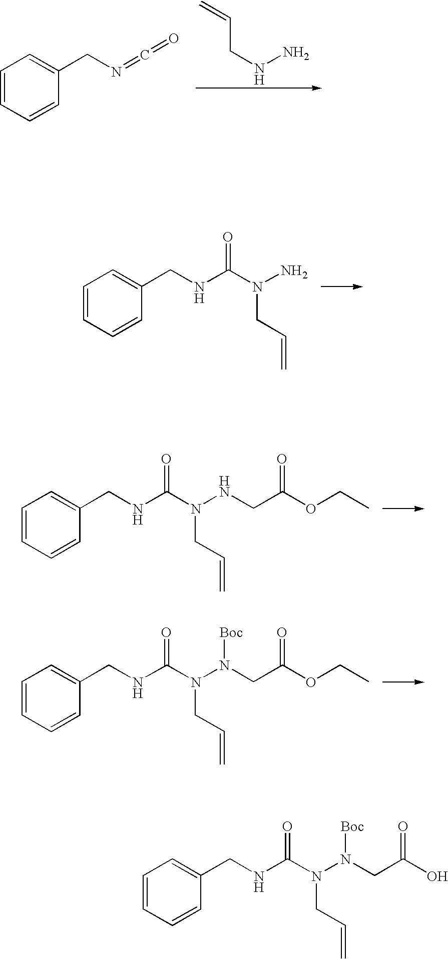

### Step-1 (4-Benzyl-2-allylsemicarbazide)

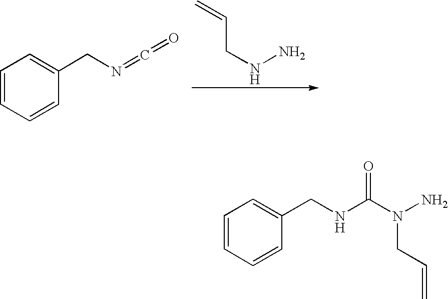

To the solution of Allyl hydrazine (1.55 mL, 15.0 mmol) in 7.5 mL of CHCl₃ was added benzyl isocyanate (1.85 mL, 15.0 mmol) slowly at 0° C. and stirred at the same temperature for 2 h. The resulting mixture was dissolved in 1N HCl (200 mL) and the solution was washed with CHCl₃ (50 mL×3). The aqueous phase was adjusted to pH 12 with 2 M NaOH aq and then extracted with CHCl₃ (100 mL×3). The organic phase was dried over Na₂SO₄ and concentrated in vacuo. The residue was recrystallized from hexane-CHCl₃ to afford a colorless crystal (2.20 g, 10.7 mmol, 70%). R_{f}=0.50 (CHCl₃:MeOH=9:1); ¹H NMR (400 MHz, CDCl₃) [symbol] 7.34-7.23 (5H, m), 6.77 (1H, br s), 5.77 (1H, ddt, J=16.9, 10.1, 6.3 Hz), 5.28 (1H, d, J=10.1 Hz), 5.22 (1H, dd, J=16.9, 1.5 Hz), 4.42 (2H, d, J=6.3 Hz), 4.14 (2H, d, J=6.3 Hz), 3.47 (2H, s); ¹³C NMR (99.5 MHz, CDCl₃) δ 159.0, 139.9, 132.7, 128.6, 127.6, 127.2, 119.2, 52.8, 44.3; LC/MS [ESI+] (m/z) 206.3 (M+1)⁺.

### Step-2 (Ethyl 4-benzyl-2-allylsemicarbazidylacetate)

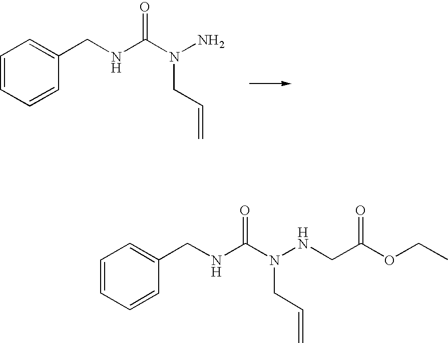

To the solution of 4-Benzyl-2-allylsemicarbazide (8.60 g, 41.9 mmol) in toluene (50 mL) were added DIPEA (14.6 mL, 83.8 mmol) and Ethyl bromoacetate (8.1 mL, 73 mmol) and then stirred at 95δ for 39 hr. The reaction mixture was allowed to cool to room temperature followed by dilution with EtOAc (150 mL). The mixture was washed with H₂O (50 mL) and brine (50 mL), dried over Na₂SO₄, filtered and concentrated. The crude was submitted to silica gel (250 g) column chromatography with Hex:EtOAc=2:1 to 1:1 as eluate to afford a pale yellow oil (7.60 g, 26.1 mmol, 62%). Rf=0.30 (Hex:EtOAc=2:3); ¹H NMR (400 MHz, CDCl₃) δ 7.32-7.23 (5H, m), 7.02 (1H, br, s), 5.78 (1H, ddt, J=17.4, 10.1, 6.3 Hz), 5.25 (2H, m), 4.42 (2H, d, J=5.8 Hz), 4.16 (3H, q and br m, J=7.2 Hz), 3.98 (1H, t, J=4.8 Hz), 3.55 (2H, d, J=4.8 Hz), 1.25 (3H, t, J=7.2 Hz); ¹³C NMR (99.5 MHz, CDCl₃) δ 170.5, 158.9, 139.8, 132.5, 128.5, 127.6, 127.1, 119.2, 61.3, 50.0, 46.7, 44.3, 14.1; LC/MS [ESI+] (m/z) 292.3 (M+1)⁺.

### Step-3 (Ethyl 4-benzyl-3-Boc-2-allylsemicarbazidylacetate)

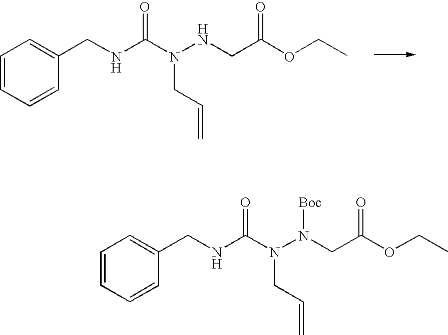

To the solution of Ethyl 4-benzyl-2-allylsemicarbazidylacetate (7.10 g, 24.4 mmol) in CH₂Cl₂ (50 mL) were added DIPEA (8.5 mL, 49 mmol), DMAP (1.19 g, 9.76 mmol) and (Boc)₂O (10.6 g, 48.8 mmol). After the mixture was stirred for 3.5 hr at room temperature, additional DIPEA (2.12 mL, 12.2 mmol) and (Boc)₂O (2.66 g, 12.2 mmol) were added. After the reaction mixture was stirred for additional 6 hr, the mixture was diluted with CH₂Cl₂ (100 mL) and then sat.NaHCO₃ (50 mL) was added at 0δ. The separated aqueous phase was extracted with CH₂Cl₂ (100 mL×2). The combined organic phases were washed with H₂O (100 mL) and brine (100 mL), dried over Na₂SO₄, filtered and concentrated. The crude was submitted to SiO₂ (300 g) column chromatography with Hex:EtOAc=7:1 to 1:1 as eluate to afford product as a pale yellow oil (6.61 g, 16.9 mmol, 69%). Rf=0.57 (Hex:EtOAc=1:1); ¹H NMR (400 MHz, CDCl₃) δ 7.77 (1H, br s), 7.34-7.21 (5H, br m), 5.88 (1H, br m), 5.20 (2H, br m), 4.62-4.46 (3H, m), 4.37-4.13 (3H, m), 3.92-3.65 (2H, m), 1.48 and 1.38 (9H, s), 1.26 (3H, t, J=7.2 Hz); ¹³C NMR (99.5 MHz, CDCl₃) δ 170.8, 157.8, 154.1, 139.8, 128.4, 127.6, 127.0, 119.6, 82.7, 62.0, 51.2, 44.3, 30.9, 28.0, 14.1; LC/MS [ESI+] (m/z) 392.4 (M+1)⁺.

### Step-4 (4-Benzyl-3-Boc-2-allylsemicarbazidylacetatic acid)

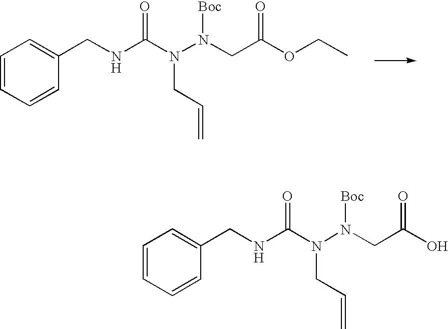

To the solution of Ethyl 4-benzyl-3-Boc-2-allylsemicarbazidylacetate (3.20 g, 8.17 mmol) in THF/MeOH/H₂O (2/3/1, 25 mL) was added LiOH H₂O (685 mg, 16.3 mmol) at 0δ. After stirred for 40 min at room temperature, the reaction mixture was diluted with CH₂Cl₂ (50 mL) at δ8. The mixture was acidified with 1N HCl and then extracted with CH₂Cl₂. The combined extraction were washed with H₂O (30 mL) and Brine (30 mL), dried over Na₂SO₄, added Et₃N (3 mL), filtered and concentrated. The crude was submitted to SiO₂ column chromatography with CHCl₃:MeOH=100:0 to 85:15 as eluate to afford orange sticky oil 4-Benzyl-3-Boc-2-allylsemicarbazidylacetatic acidδEt₃N salt (3.66 g, 7.87 mmol, 96%); ¹H NMR (400 MHz, CDCl₃, rotamer) δ 9.44 and 9.34 (1H, br s), 7.35-7.18 (5H, m), 5.91 (1H, m), 5.17 (2H, m), 4.58 and 4.87 (2H, dd, J=15.5, 6.3 and 14.5, 5.8 Hz), 4.39-4.23 (2H, m), 3.89 and 3.80 (1H, dd, J=14.0, 8.2 and 14.5, 8.2 Hz), 3.58 and 3.52 (1H, d, J=17.4 and 16.9 Hz), 2.81 (5H, q, J=7.2 Hz, Et₃N), 1.44 and 1.42 (9H, s), 1.11 (7.5H, t, J=7.2 Hz, Et₃N); ¹³C NMR (99.5 Hz, CDCl₃) δ 158.9, 154.3, 153.6, 140.6, 134.2, 128.1, 127.4, 126.5, 118.8, 81.1, 55.6, 51.4, 44.9 (Et₃N), 44.2, 28.2, 8.3 (Et₃N); LC/MS [ESI+] (m/z) 364.3 (M+1)⁺.

Synthesis of Compound No. 61

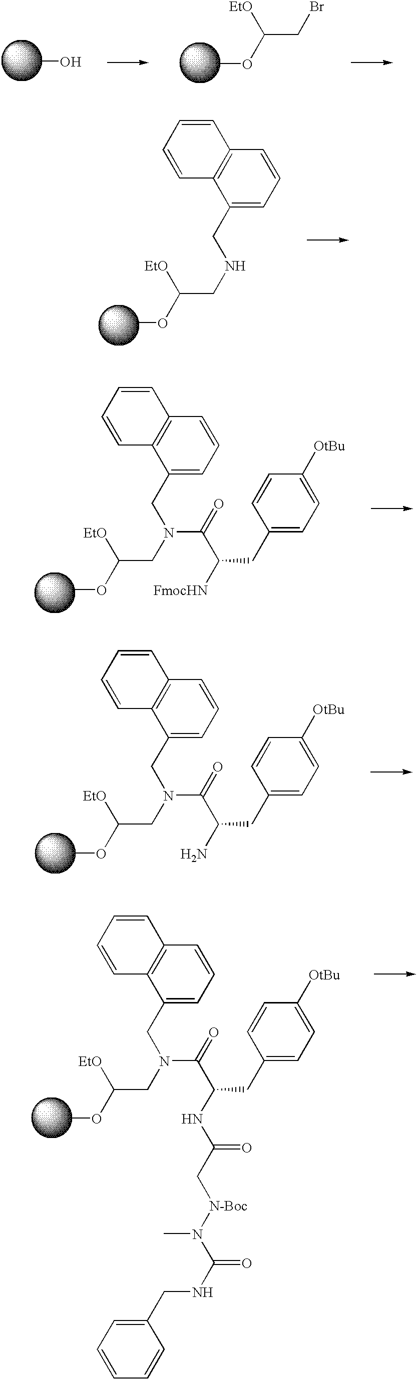

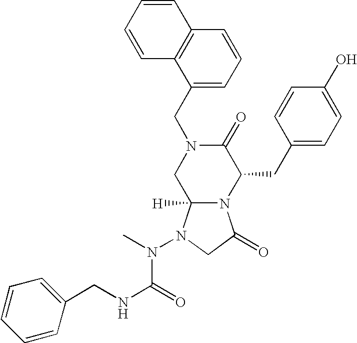

### Step-1

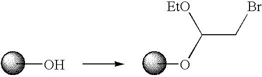

The hydroxy-functionalized resin (5.0 g, 0.68 mmol/g, Novabiochem) was placed in 200 mL round-bottom flask. To the mixture of the resin and PPTS (1.7 g, 6.8 mmol) in 1,2-dichloromethane (51 mL) was added bromoacetaldehyde diethylacetal (4.2 mL, 27 mmol) at room temperature. After being stirred under reflux for 4.0 hr, the mixture was filtered and the resin was washed with DMF 50 mL×3, DMSO 50 mL×3, 1,4-dioxane 50 mL×3, CH₂Cl₂ 50 mL×3, MeOH 50 mL×3, Et₂O 50 mL×3. The resin was dried under reduced pressure for over night to afford the desired bromoacetyl resin (5.5 g).

### Step-2

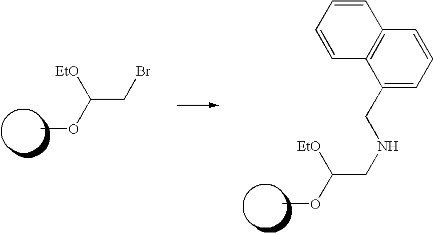

Bromoacetyl resin (1.0 g, 0.9 mmol/g) was placed in 30 mL round-bottom flask. The resin was swollen with DMF (9.0 mL×5 min×1) and then treated with 1.0 M solution of 1-naphtylmethylamine (1.4 g, 9.0 mmol) in DMSO (9.0 mL) at 70° C. After being stirred for 12 hr, the resin was filtered and rinsed with DMSO (9.0 mL×5 min×3). The resin was washed with DMF (5.0 mL×5 min×3) and CH₂Cl₂ (5.0 mL×5 min×3). The resin was dried under reduced pressure to afford desired resin (1.18 g).

### Step-3

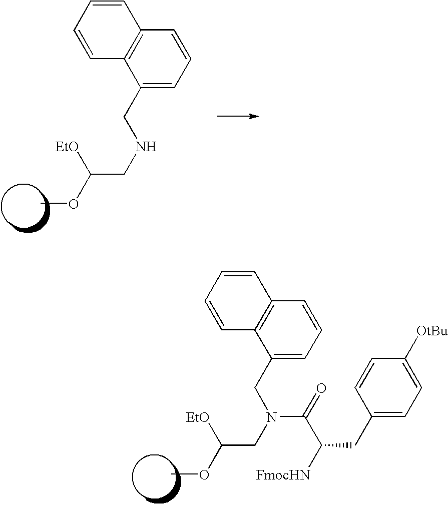

Naphthylmethylamino resin (1.18 g, 0.84 mmol/g) was placed in 20 mL plastic disposable syringe. The resin was swollen with DMF (9.0 mL×5 min×1) and then DMF (9.0 mL), Fmoc-Tyr(t-Bu % OH (620 mg, 1.35 mmol), DIPEA (470 μL, 2.70 mmol) and HATU (513 mg, 1.35 mmol) were added at room temperature. After being shaken for 12 hr, in case of Kaiser test was positive, the same procedure was repeated. The mixture was filtered and the resin was washed with DMF (10.0 mL×5 min×3) and CH₂Cl₂ (10.0 mL×5 min×3). The resin was dried under reduced pressure to afford desired resin (1.50 g).

### Step-4

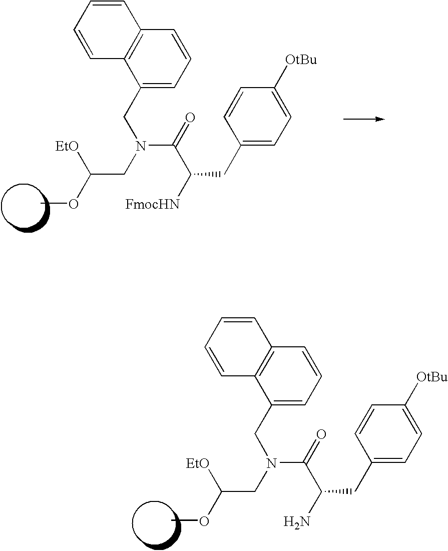

The 1-Naphthylmethylamino-Fmoc-Tyr(tBu) resin (1.50 g, 0.61 mmol/g) was placed in 20 mL plastic disposable syringe. The resin was swollen in DMF (10.0 mL) and DM was sucked out. The resin was treated with 20 v/v % piperidine/DMF (10.0 mL) at room temperature. After being shaken for 1.0 hr, the mixture was filtered and the resin was washed with DMF (10 mL×5 min×3) and CH₂Cl₂ (10 mL×5 min×3). The resin was dried under reduced pressure to afford desired resin (1.48 g).

### Step-5

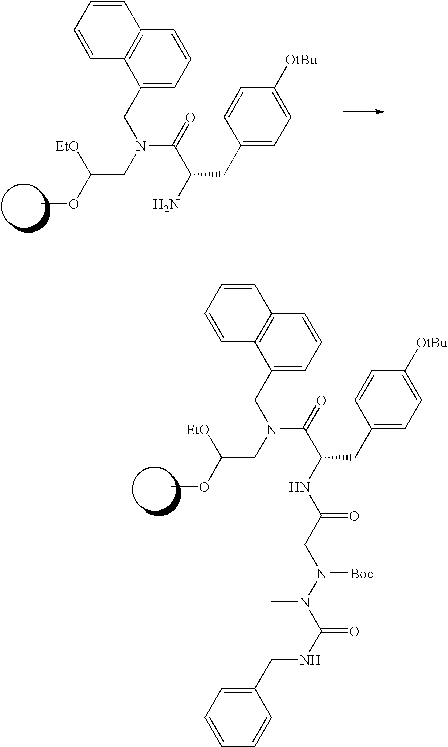

The Amino resin (300 mg, 0.71 mmol/g) was placed in 20 mL plastic disposable syringe. The resin was swollen in DMF (3.0 mL) and DMF was sucked out. To the resin was added 0.3 M stocked CH₂Cl₂ solution of 4-Benzyl-3-Boc-2-methylsemicarbazidylacetatic acid (2.5 mL, 0.75 mmol), DIPEA (260 μL, 1.49 mmol) and HATU (284 mg, 0.75 mmol) at room temperature. After being shaken for 12 hr, the mixture was filtered and the resin was washed with DMF (5.0 mL×5 min×3) and CH₂Cl₂ (5.0 mL×5 min×3). The resin was dried under reduced pressure to afford desired resin.

### Step-6

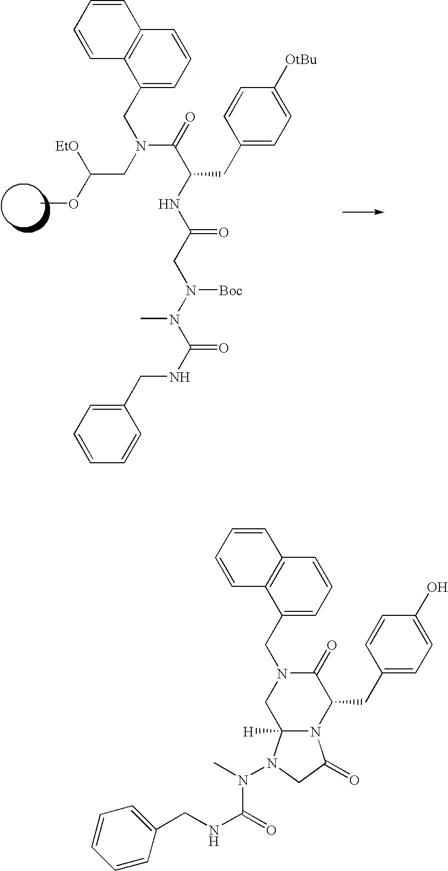

The resin (115 mg, 0.58 mmol/g) was placed in 5.0 mL plastic disposable syringe. After addition of 99% HCO₂H (1.0 mL), the mixture was shaken for 12 hr at room temperature, the solution was collected by filtration. The resin was washed with 99% HCO₂H (1.5 mL×5 min×2). The combined HCO₂H solutions were concentrated and then submitted to silica gel column chromatography to afford Compound No. 61 (7.1 mg, 19% from bromoacetyl resin). Rf=0.63 (CHCl₃:MeOH=9:1); ¹H NMR (400 MHz, CDCl₃) δ 8.06 (1H, d, J=8.2 Hz), 7.89 (1H, m), 7.84 (1H, d, J=8.2 Hz), 7.56 (2H, m), 7.38 (1H, dd, J=8.2, 7.2 Hz), 7.20 (3H, m), 7.12 (1H, d, J=6.8 Hz), 7.05 (2H, dd, J=7.7, 2.9 Hz), 7.02 (2H, d, J=8.2 Hz), 6.88 (0.5H, br s), 6.71 (2H, d, J=8.2 Hz), 6.05 (1H, t, J=5.8 Hz), 5.06 (2H, ABq, J=14.5 Hz), 4.80 (1H, dd, J=5.8, 2.5 Hz), 4.23 (2H, ABX, J=14.5, 5.8 Hz), 3.67-3.44 (4H, m), 3.21 (1H, dd, J=14.0, 5.8 Hz), 3.12 (1H, dd, J=11.0, 3.9 Hz), 2.86 (1H, dd. J=11.0, 9.1 Hz), 2.59 (3H, s); LC/MS [ESI+] (m/z) 564.4 (M+1)⁺.

Synthesis of Compound No. 71

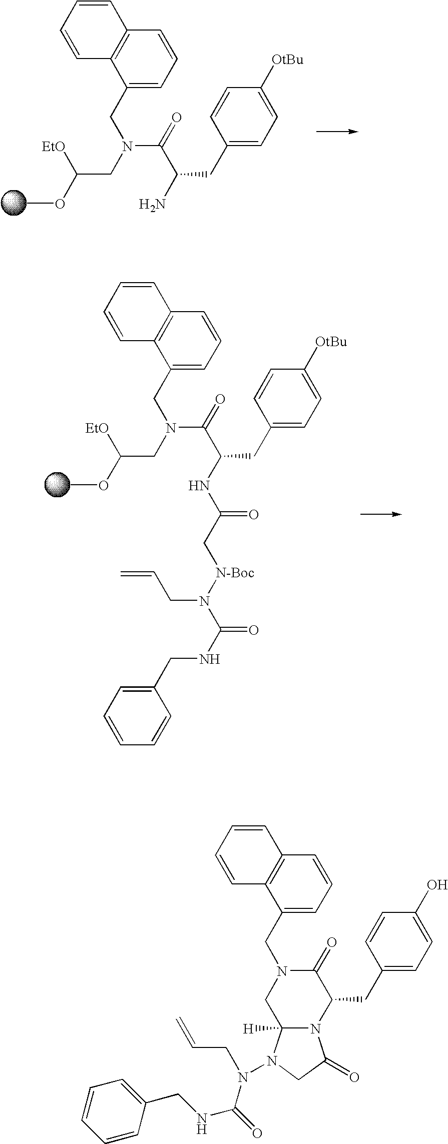

Step-1

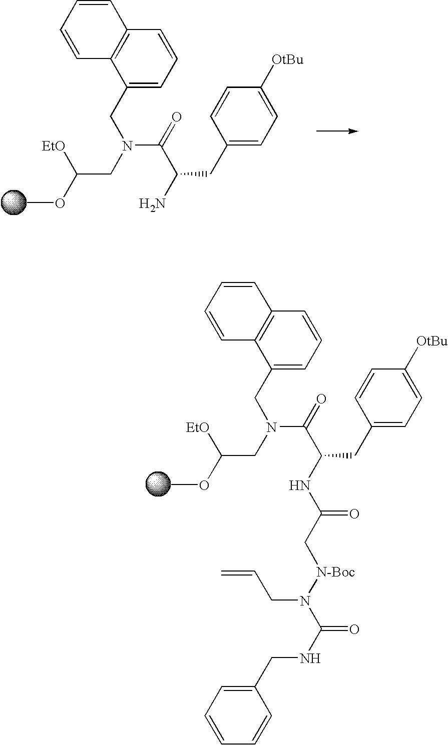

The Amino resin (100 mg, 0.71 mmol/g) was placed in 5 mL plastic disposable syringe. The resin was swollen in DMF (1.0 mL) and DMF was sucked out. To the resin was added 0.3 M stocked CH₂Cl₂ solution of 4-Benzyl-3-Boc-2-allylsemicarbazidylacetatic acid (830 μL, 0.25 mmol), DIPEA (87 μL, 0.50 mmol) and HATU (95 mg, 0.25 mmol) at room temperature. After being shaken for 12 hr, the mixture was filtered and the resin was washed with DMF (1.0 mL×5 min×3) and CH₂Cl₂ (1.0 mL×5 min×3). The resin was dried under reduced pressure to afford desired resin.

Step-2

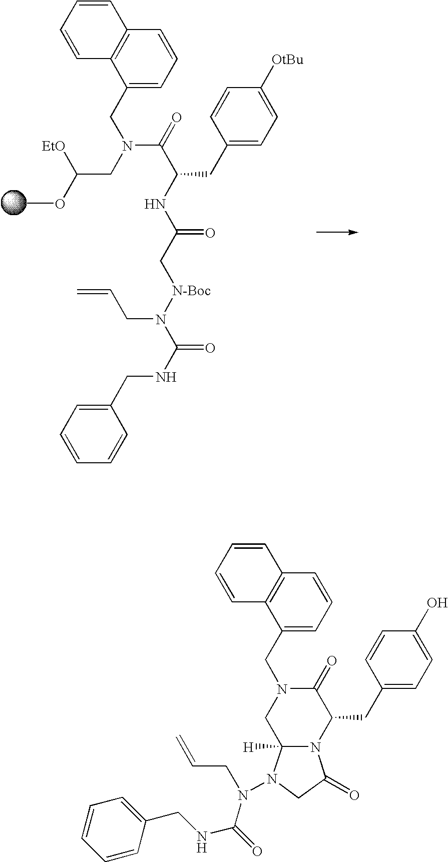

The resin (100 mg, 0.57 mmol/g) was placed in 5.0 mL plastic disposable syringe. After addition of 99% HCO₂H (1.0 mL), the mixture was shaken for 12 hr at room temperature, the solution was collected by filtration. The resin was washed with 99% HCO₂H (1.5 mL×5 min×2). The combined HCO₂H solutions were concentrated and then submitted to silica gel column chromatography to afford Compound No. 71 (11 mg, 26% from bromoacetyl resin). Rf=0.63 (CHCl₃:MeOH 9:1).

Similar synthesis was carried out to obtain the compounds as shown as Compounds 1-1200 in FIGS. 1-6.

Synthesis of Compound No. 1273

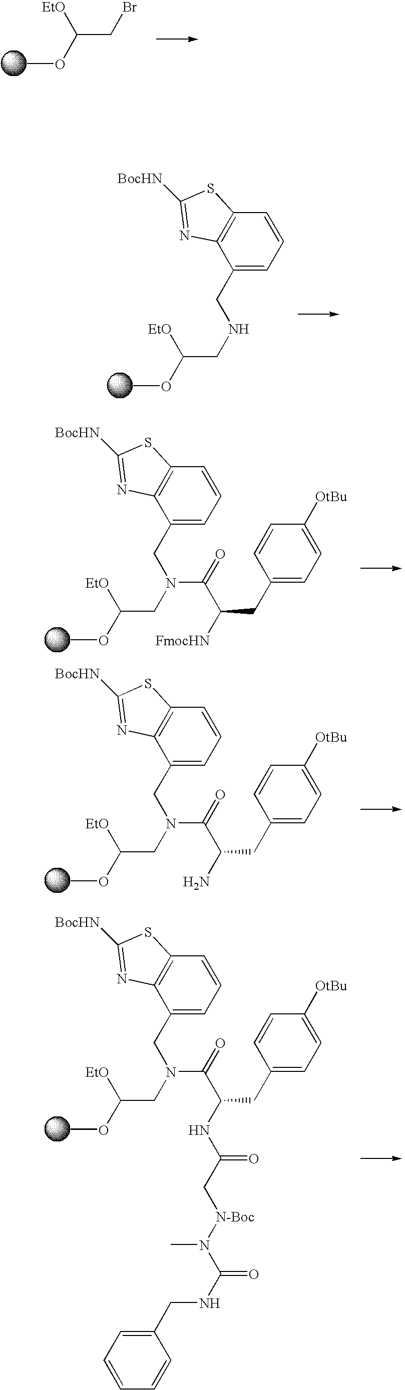

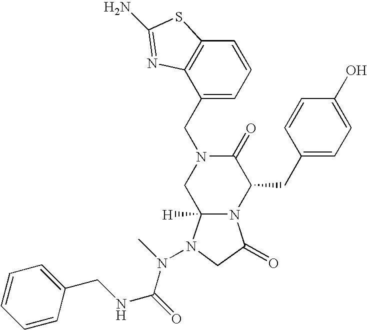

Step-1

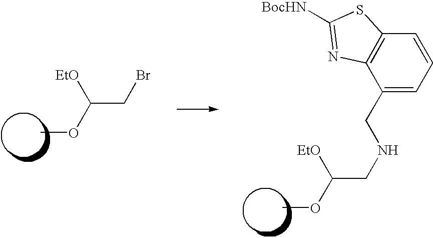

Bromoacetyl resin (1.0 g, 0.9 mmol/g) was placed in 30 mL round-bottom flask. The resin was swollen with DMF (9.0 mL×5 min×1) and then treated with 1.0 M suspension of 2-tert-Butoxycarbonylaminobenzothiazole-4-methylamine (2.5 g, 9.0 mmol) in DMSO (9.0 mL) at 70° C. After being stirred for 12 hr, the resin was filtered and rinsed with DMSO (9.0 mL×5 min×3). The resin was washed with DMF (5.0 mL×5 min×3) and CH₂Cl₂ (5.0 mL×5 min×3). The resin was dried under reduced pressure to afford desired resin (1.16 g).

Step-2

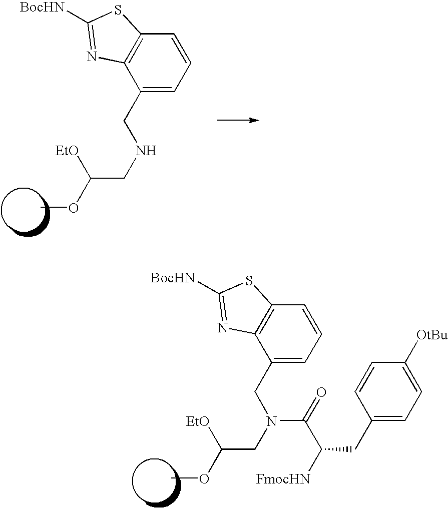

2-tert-Butoxycarbonylaminoebenzothiazole-4-methylamino resin (1.16 g, 0.76 mmol/g) was placed in 20 mL plastic disposable syringe. The resin was swollen with DMF (9.0 mL×5 min×1) and then DMF (9.0 mL), Fmoc-Tyr(t-Bu)-OH (620 mg, 1.35 mmol), DIPEA (470 μL, 2.70 mmol) and HATU (513 mg, 1.35 mmol) were added at room temperature. After being shaken for 12 hr, in case of Kaiser test was positive, the same procedure was repeated. The mixture was filtered and the resin was washed with DMF (10.0 mL×5 min×3) and CH₂Cl₂ (10.0 mL×5 min×3). The resin was dried under reduced pressure to afford desired resin (1.76 g).

Step-3

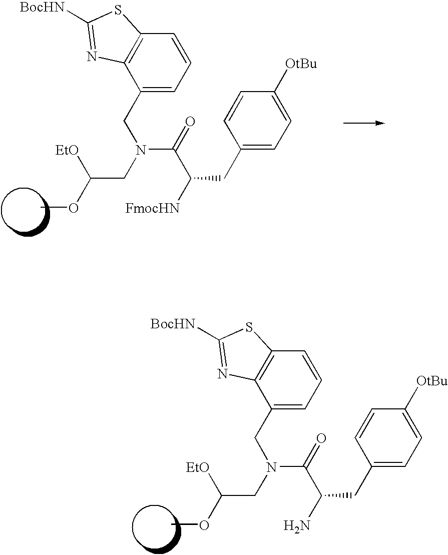

The 2-tert-Butoxycarbonylbenzothiazole-4-methylamino-Fmoc-Tyr(tBu) resin (1.76 g, 0.57 mmol/g) was placed in 20 mL plastic disposable syringe. The resin was swollen in DMF (10.0 mL) and DMF was sucked out. The resin was treated with 20 v/v % piperidine/DMF (10.0 mL) at room temperature. After being shaken for 1.0 hr, the mixture was filtered and the resin was washed with DMF (10 mL×5 min×3) and CH₂Cl₂ (10 mL×5 min×3). The resin was dried under reduced pressure to afford desired resin (1.42 g).

Step-4

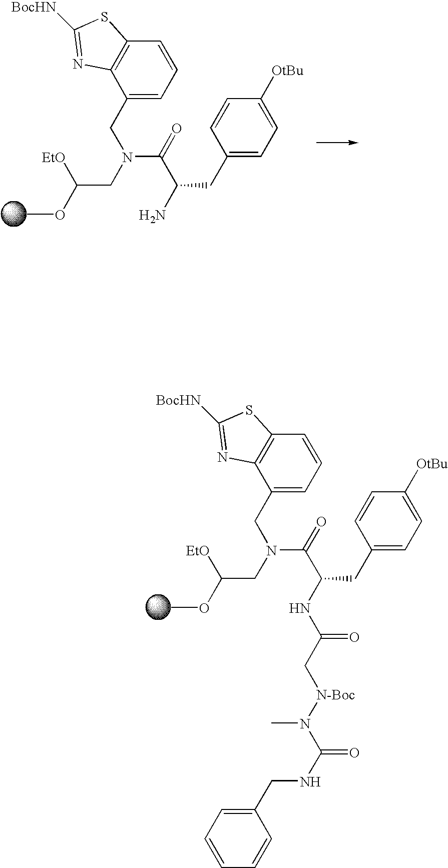

The Amino resin (350 mg, 0.65 mmol/g) was placed in 20 mL plastic disposable syringe. The resin was swollen in DMF (3.0 mL) and DMF was sucked out. To the resin was added 0.3 M stocked CH₂Cl₂ solution of 4-Benzyl-3-Boc-2-methylsemicarbazidylacetatic acid (2.7 mL, 0.80 mmol), DIPEA (277 μL, 1.59 mmol) and HATU (302 mg, 0.80 mmol) at room temperature. After being shaken for 12 hr, the mixture was filtered and the resin was washed with DM (5.0 mL×5 min×3) and CH₂Cl₂ (5.0 mL×5 min×3). The resin was dried under reduced pressure to afford desired resin.

Step-5

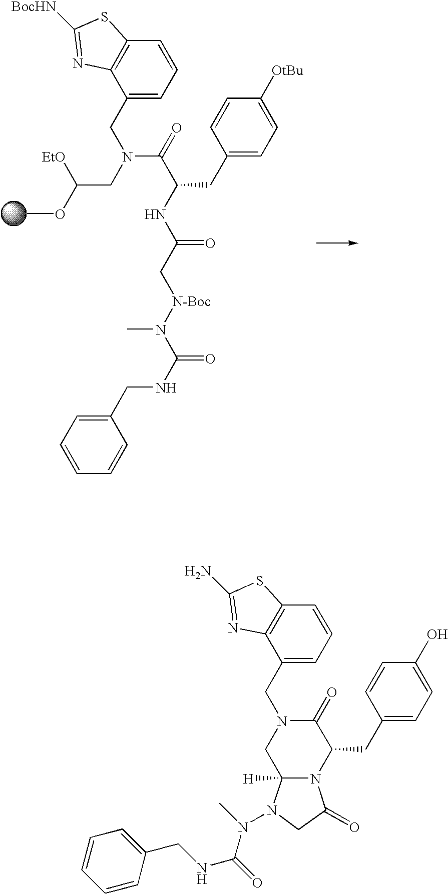

The resin (350 mg, 0.54 mmol/g) was placed in 20 mL plastic disposable syringe. After addition of 99% HCO₂H (4.0 mL), the mixture was shaken for 12 hr at room temperature, the solution was collected by filtration. The resin was washed with 99% HCO₂H (4.0 mL×5 min×2). The combined HCO₂H solutions were concentrated and then submitted to silica gel column chromatography to afford Compound No. 1273 (9.1 mg, 6.8% from bromoacetyl resin). Rf=0.47 (CHCl₃:MeOH=9:1).

Synthesis of Compound No. 1285

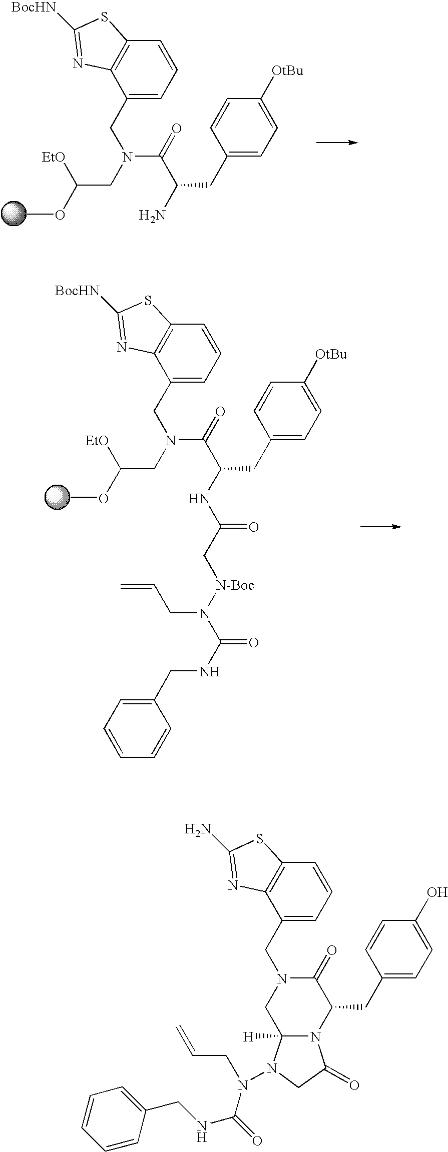

Step-1

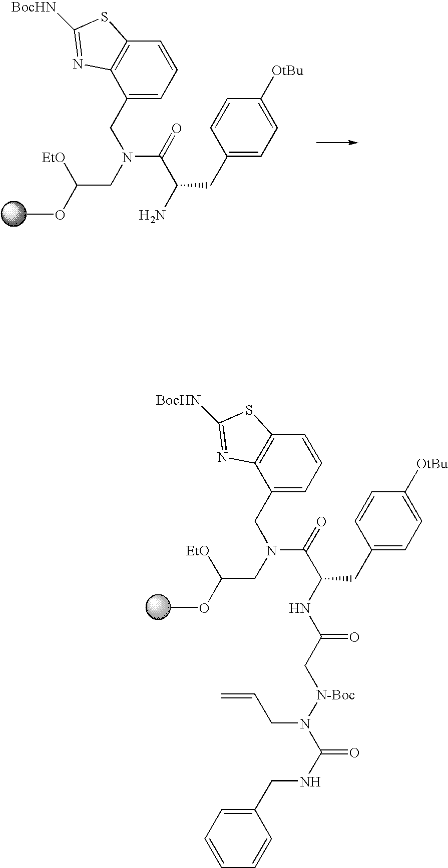

The Amino resin (350 mg, 0.65 mmol/g) was placed in 20 mL plastic disposable syringe. The resin was swollen in DMF (3.0 mL) and DMF was sucked out. To the resin was added 0.3 M stocked CH₂Cl₂ solution of 4-Benzyl-3-Boc-2-affylsemicarbazidylacetatic acid (2.7 mL, 0.80 mmol), DIPEA (277 μL, 1.59 mmol) and HATU (302 mg, 0.80 mmol) at room temperature. After being shaken for 12 hr, the mixture was filtered and the resin was washed with DMF (5.0 mL×5 min×3) and CH₂Cl₂ (5.0 mL×5 min×3). The resin was dried under reduced pressure to afford desired resin.

Step-2

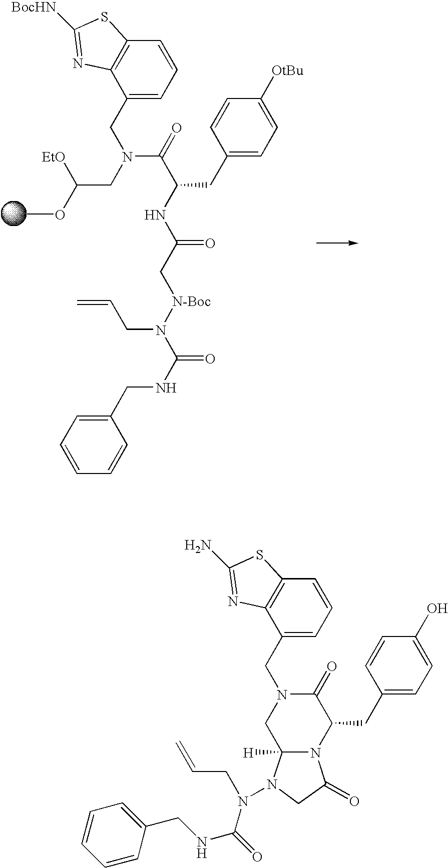

The resin (350 mg, 0.53 mmol/g) was placed in 20 mL plastic disposable syringe. After addition of 99% HCO₂H (4.0 mL), the mixture was shaken for 12 hr at room temperature, the solution was collected by filtration. The resin was washed with 99% HCO₂H (4.0 mL×5 min×2). The combined HCO₂H solutions were concentrated and then submitted to silica gel column chromatography to afford Compound No. 1285 (18 mg, 13% from bromoacetyl resin). Rf=0.52 (CHCl₃:MeOH=9:1).

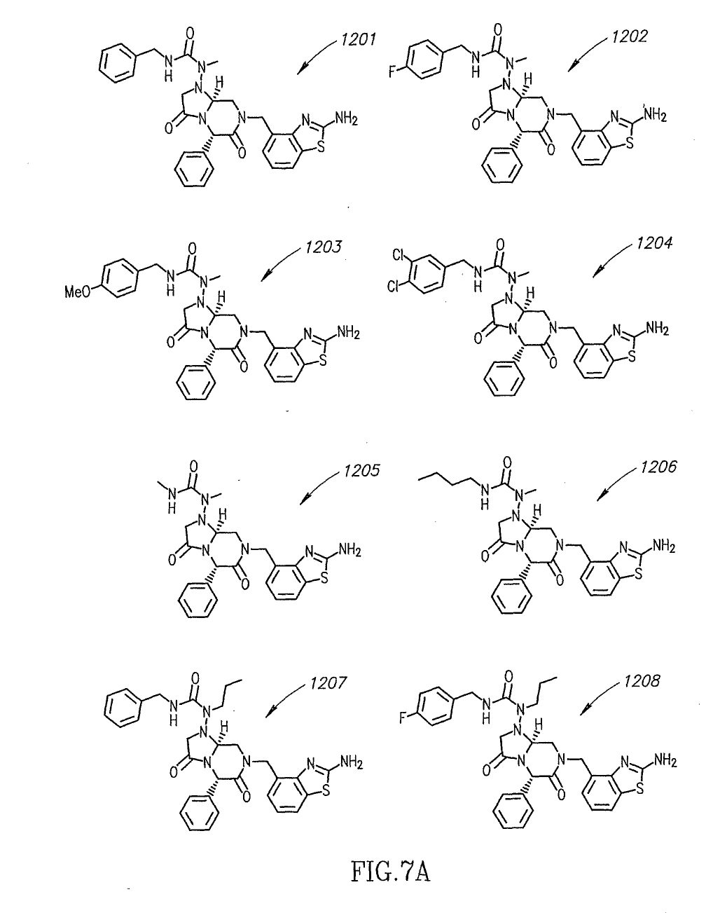

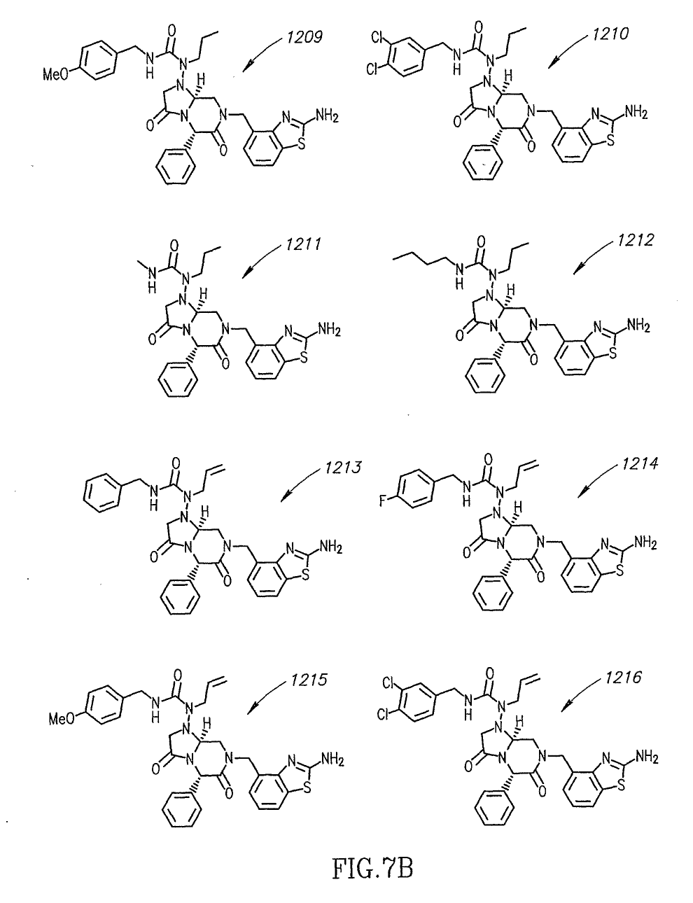

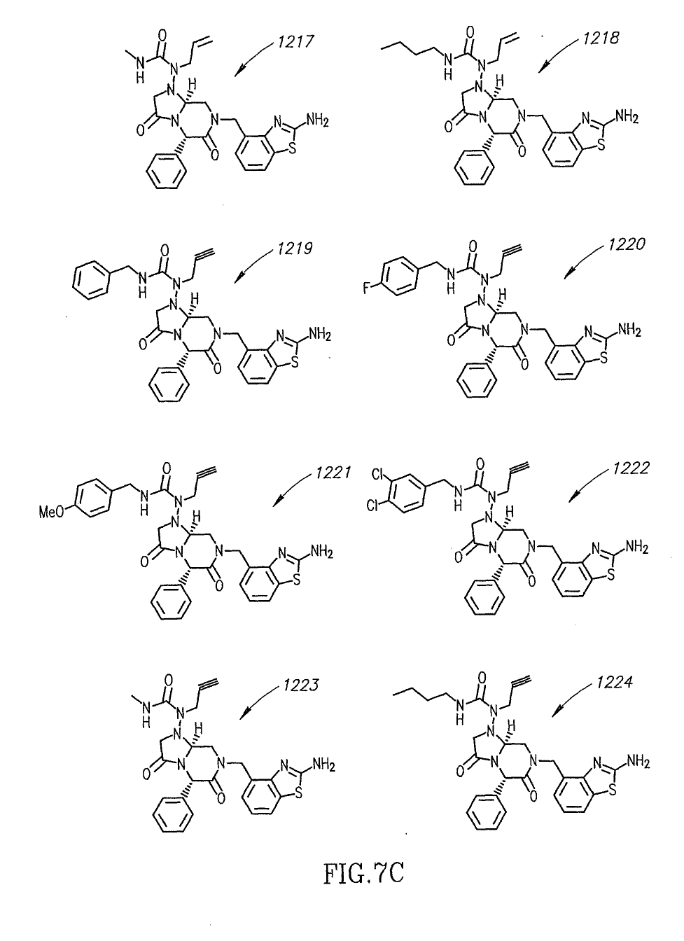

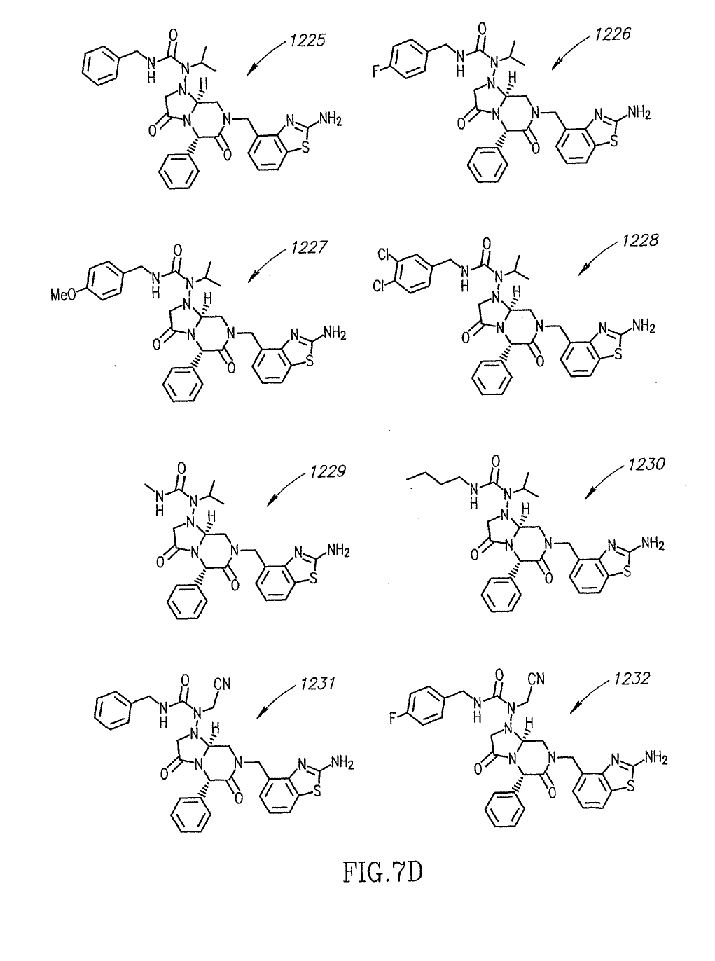

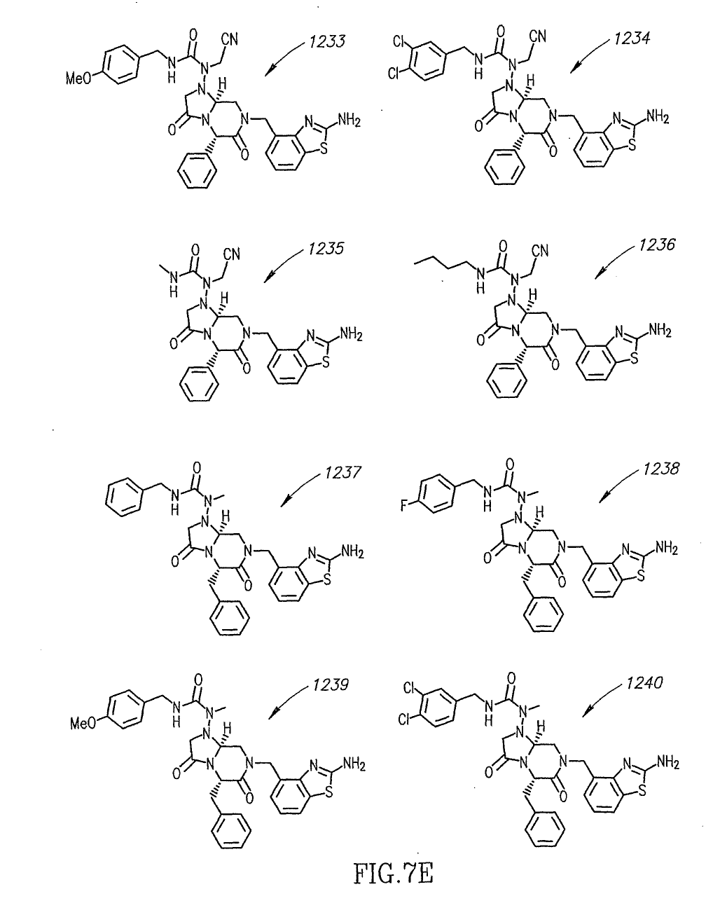

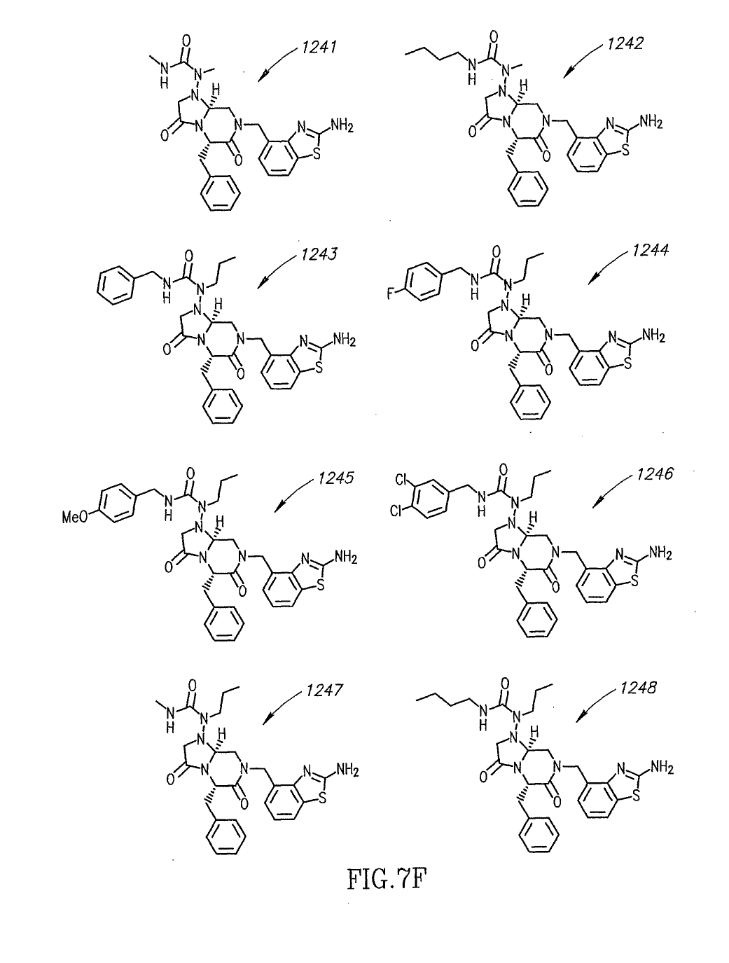

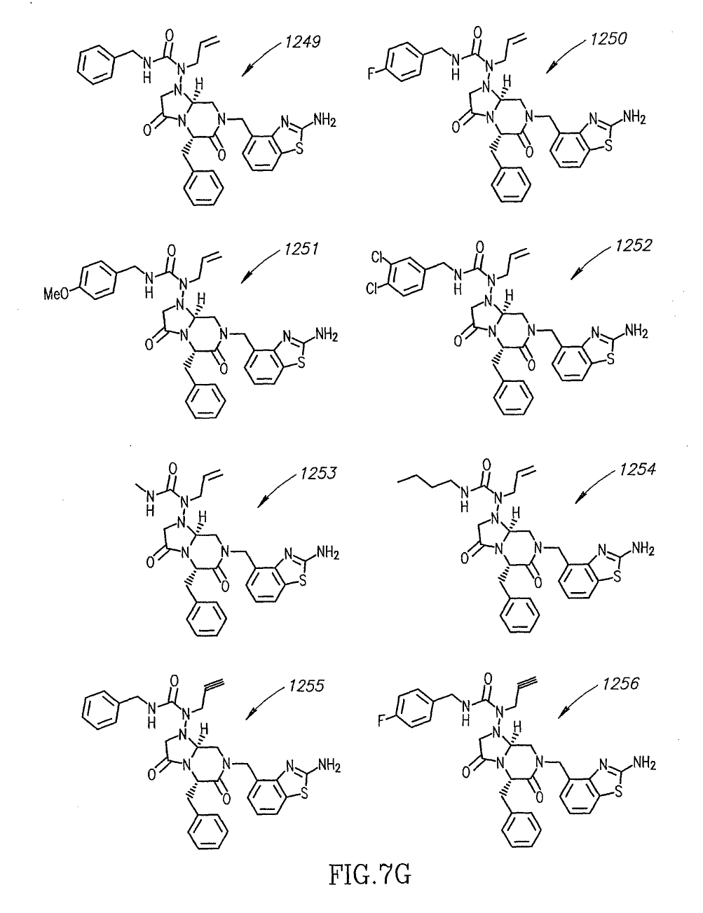

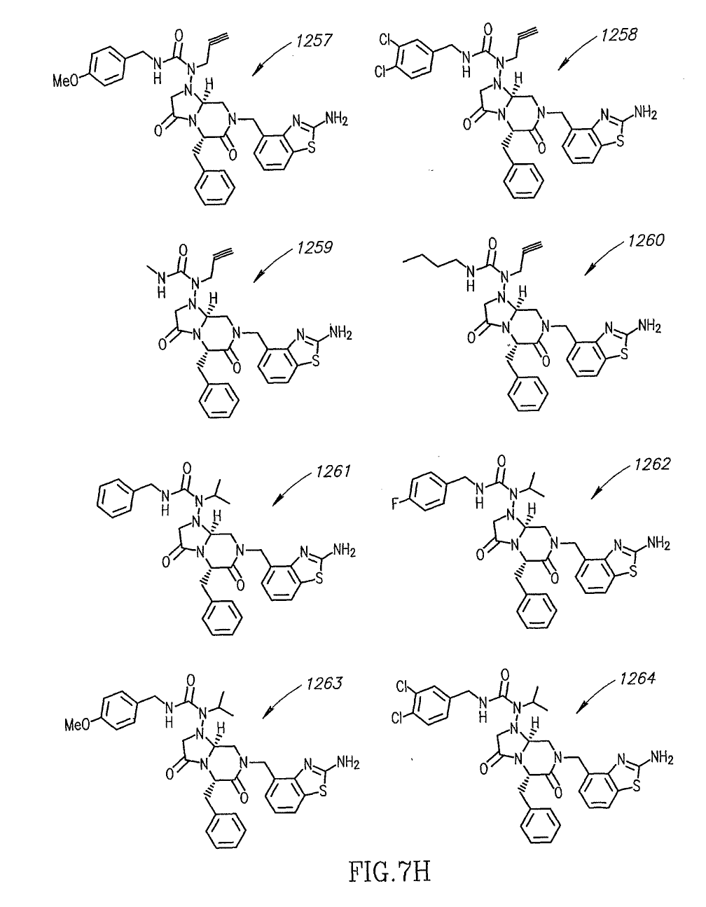

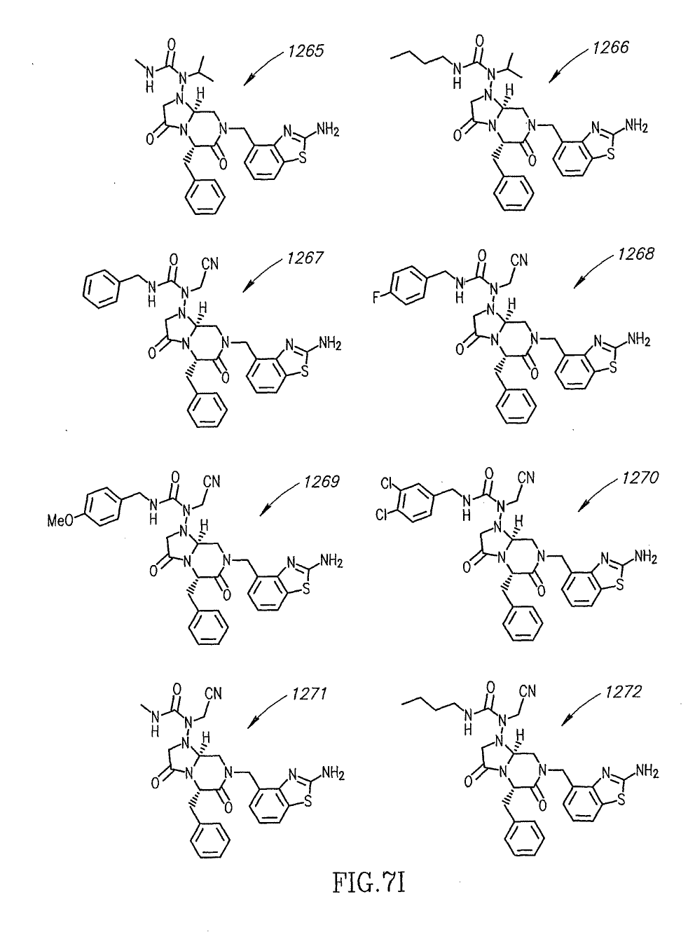

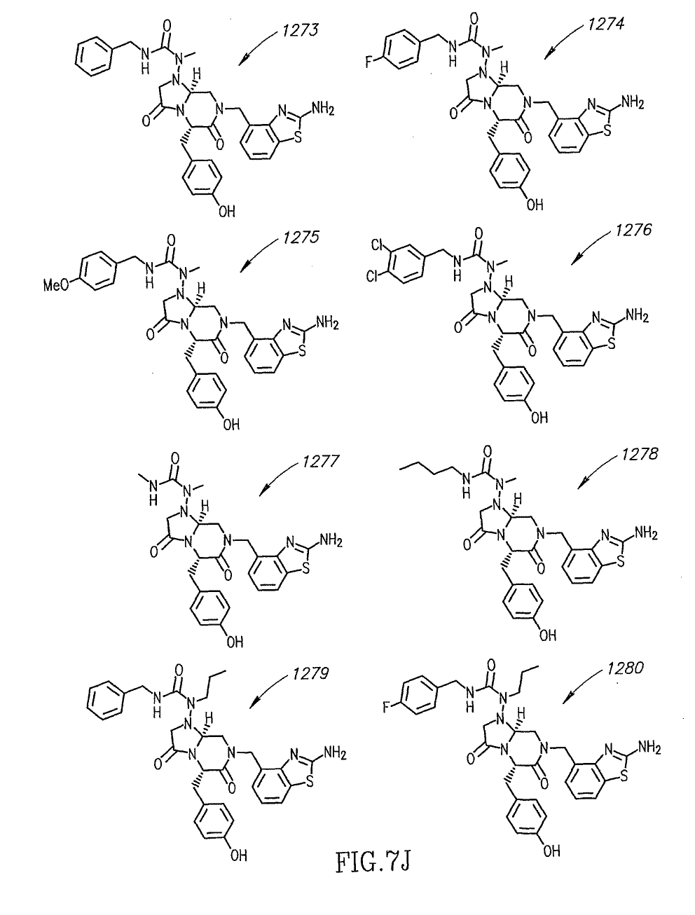

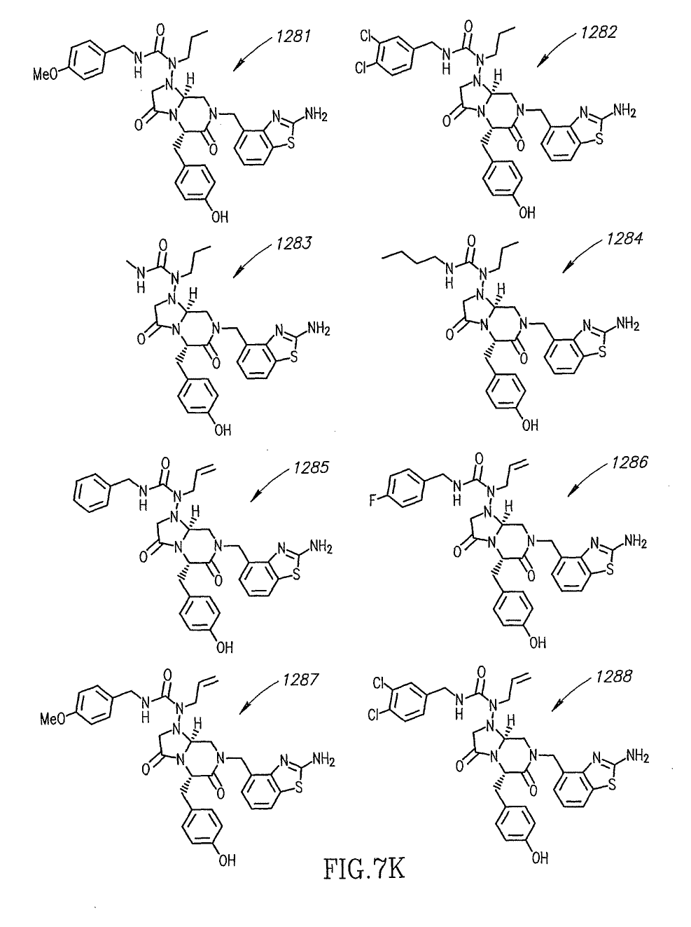

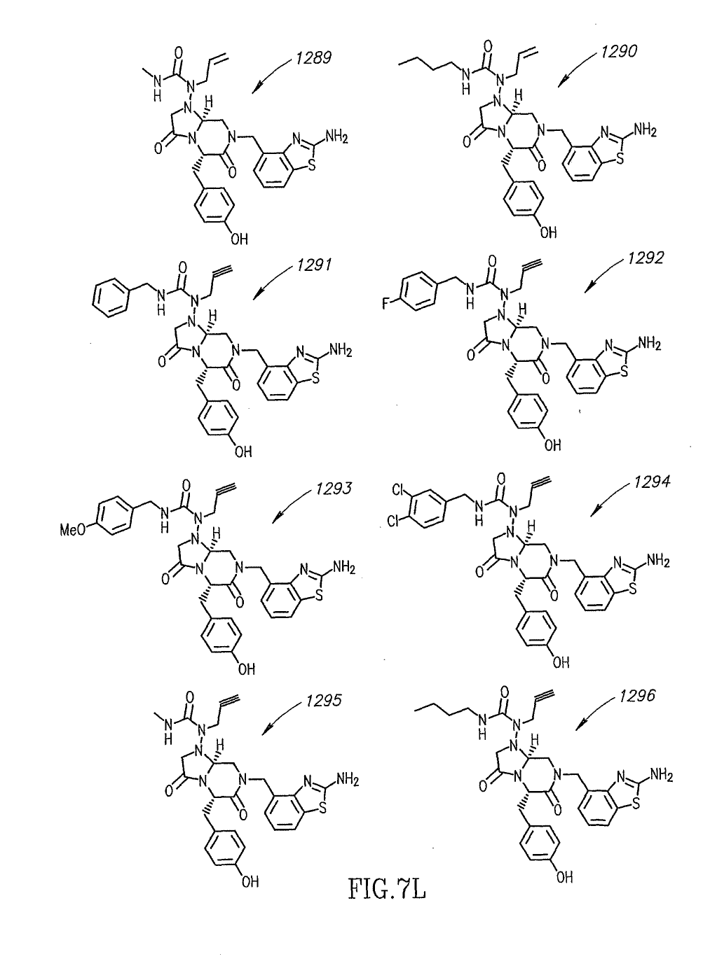

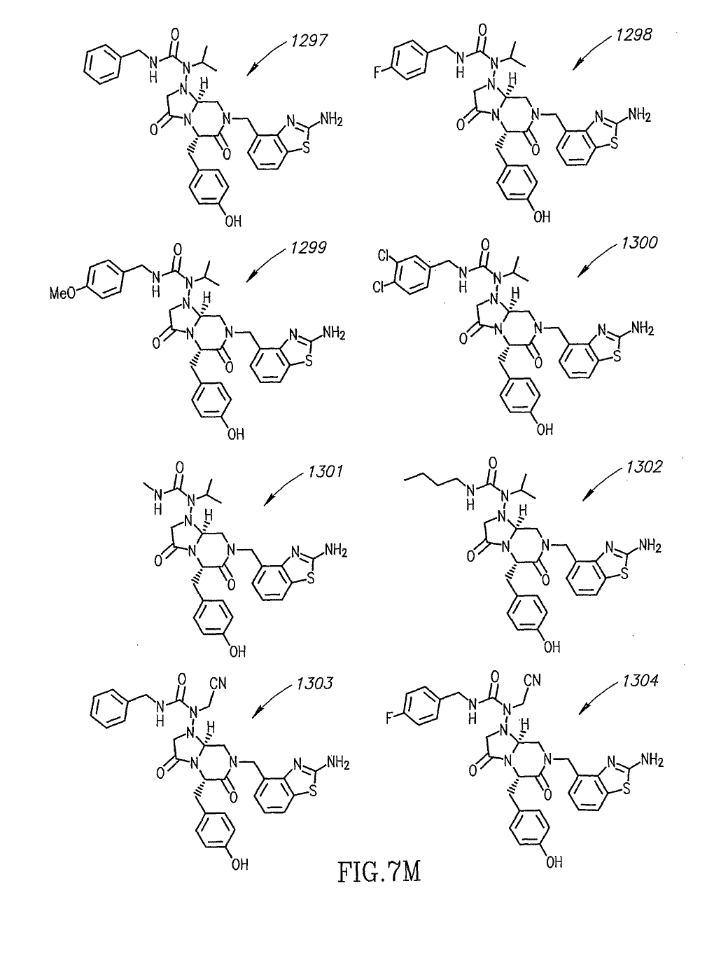

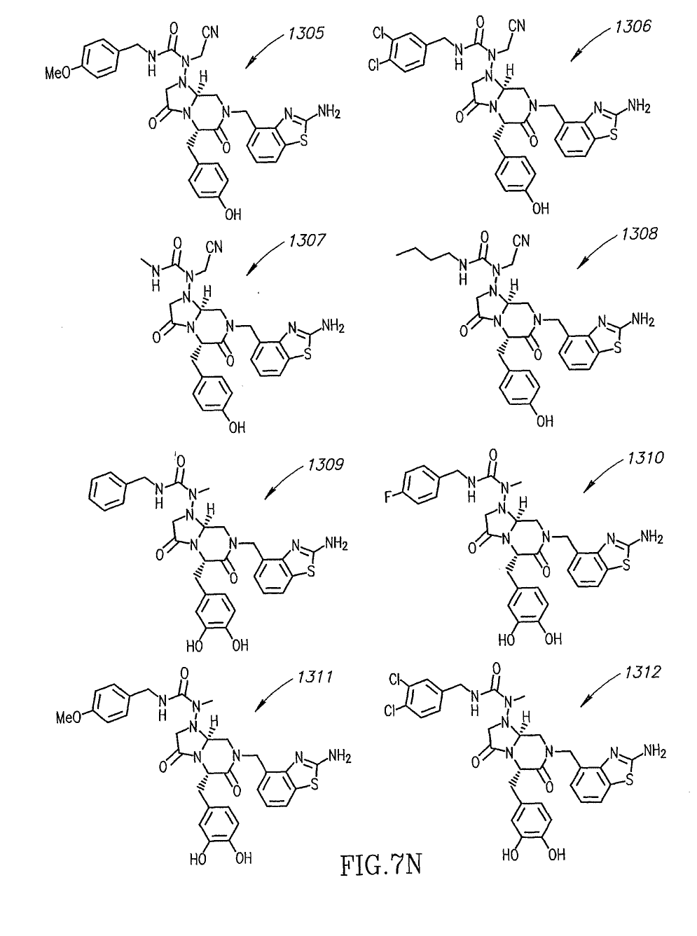

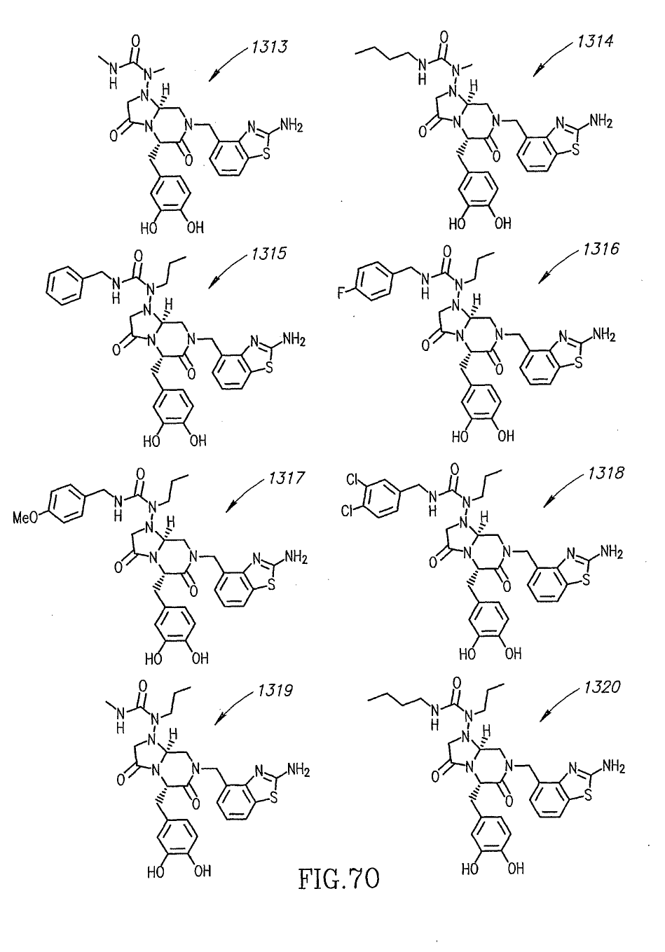

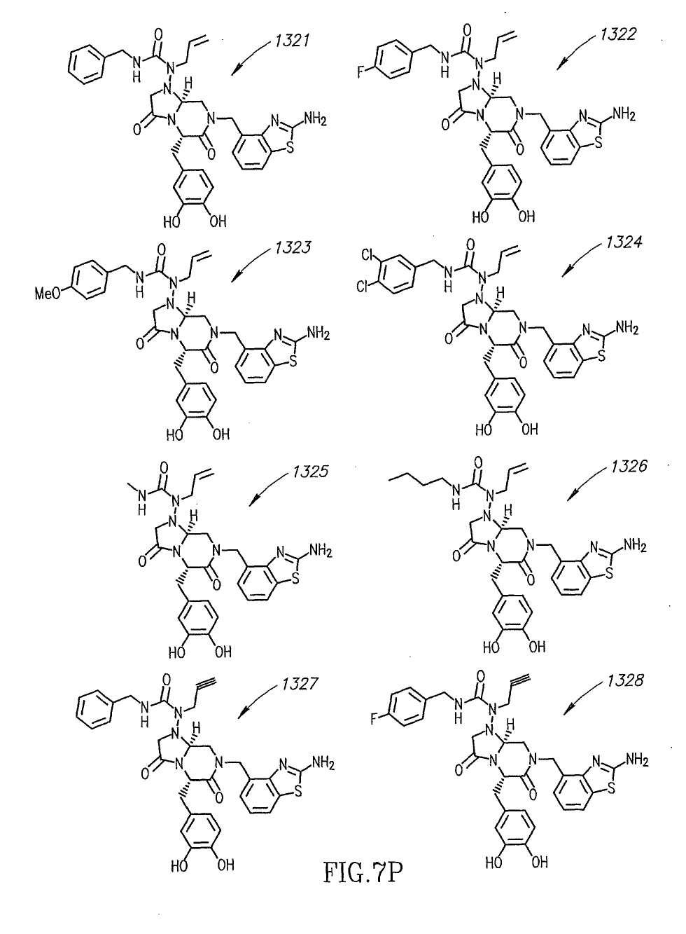

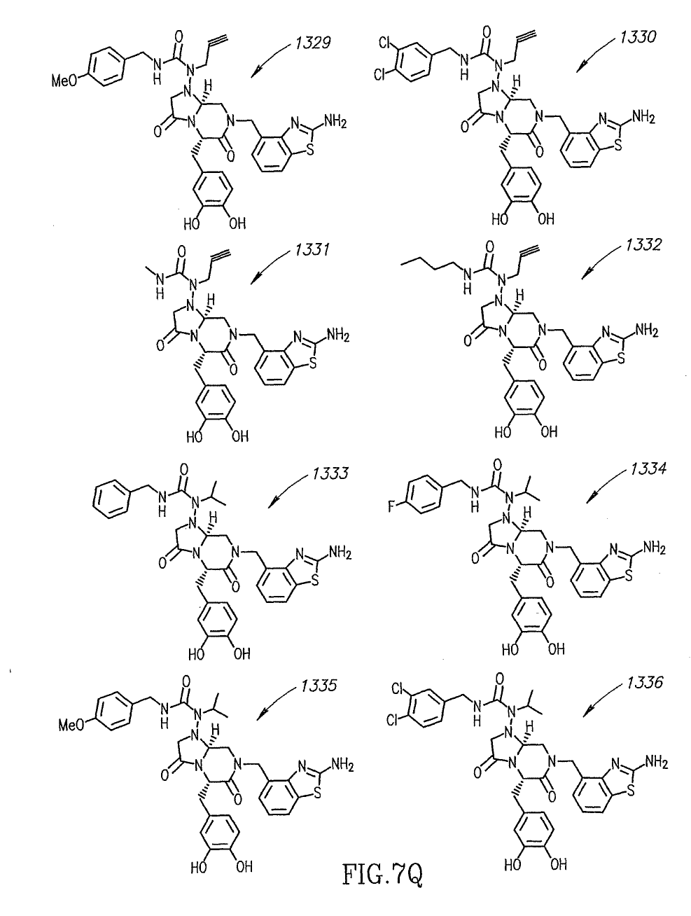

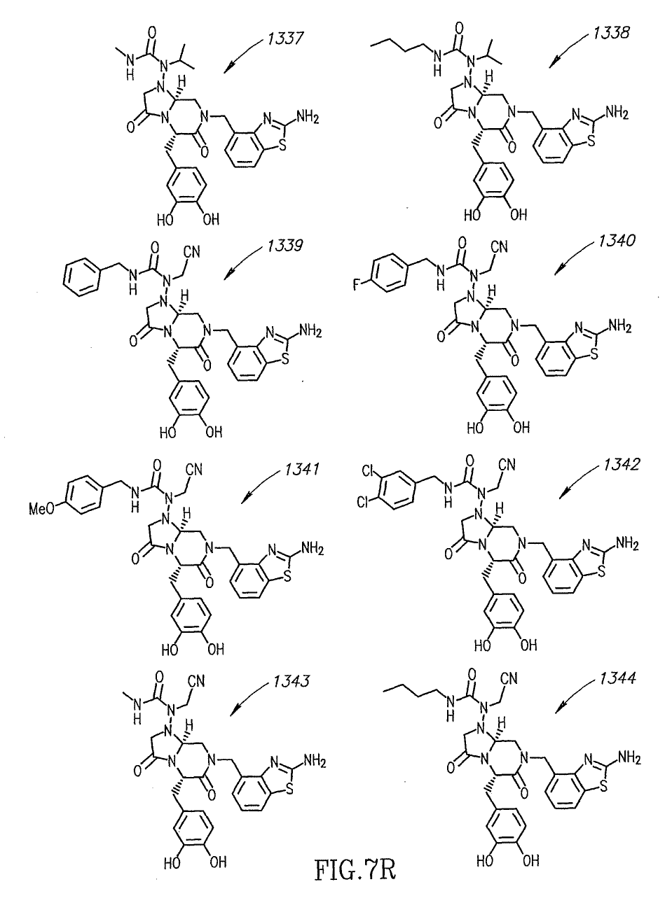

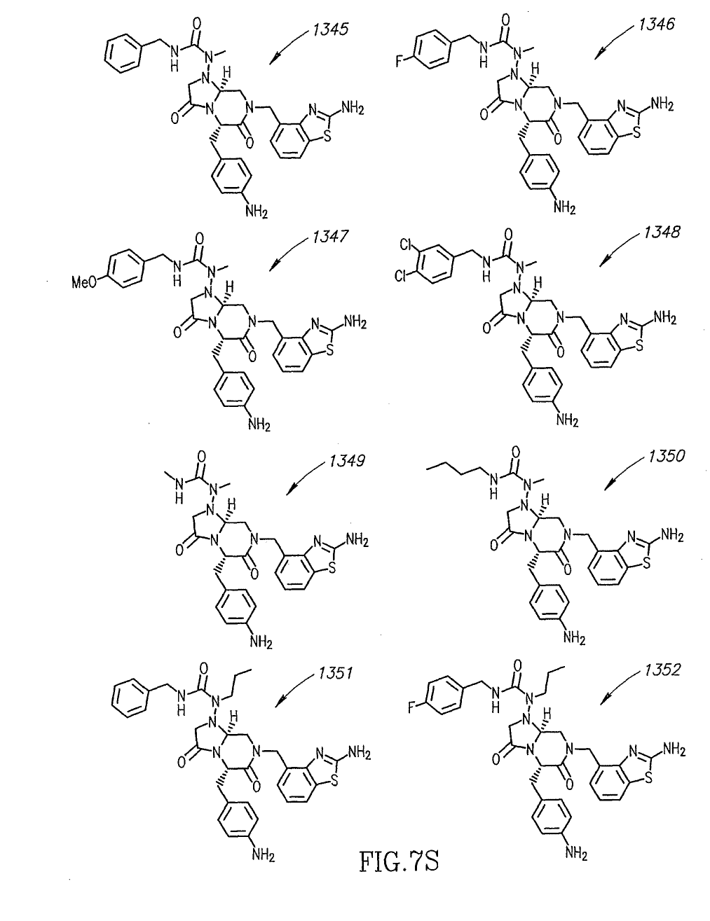

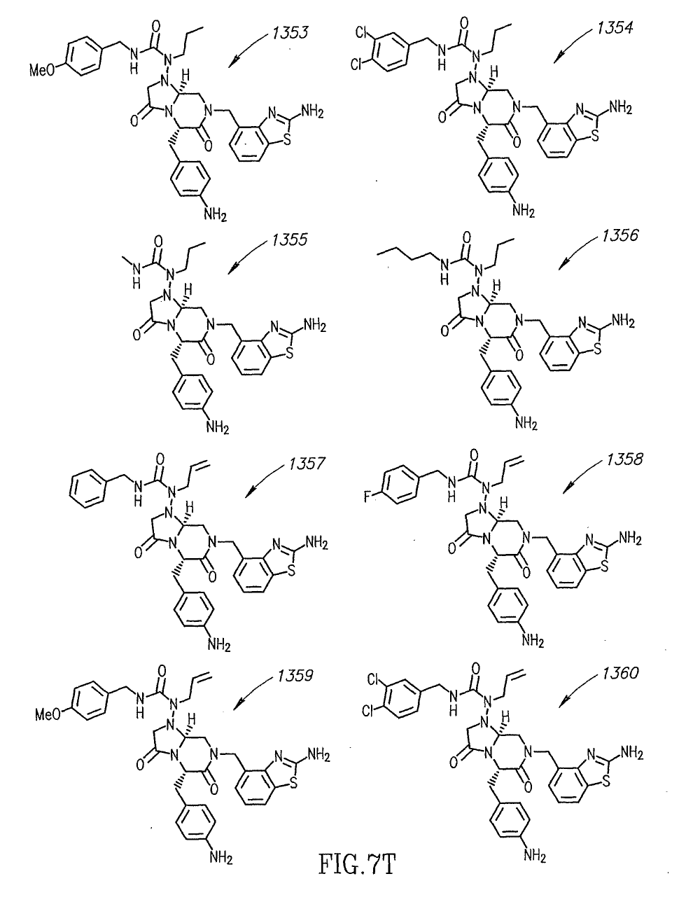

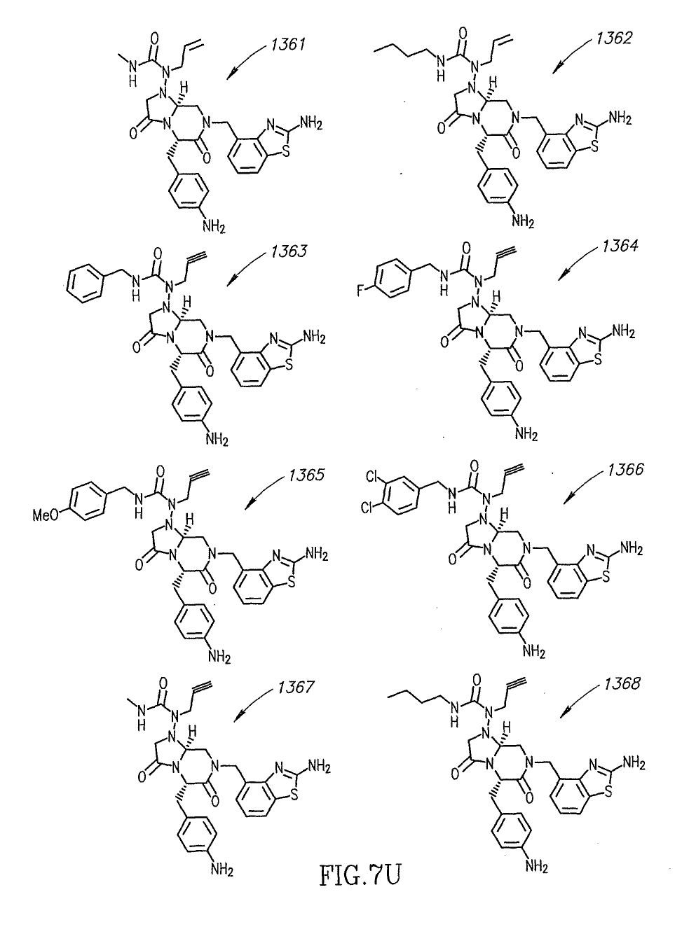

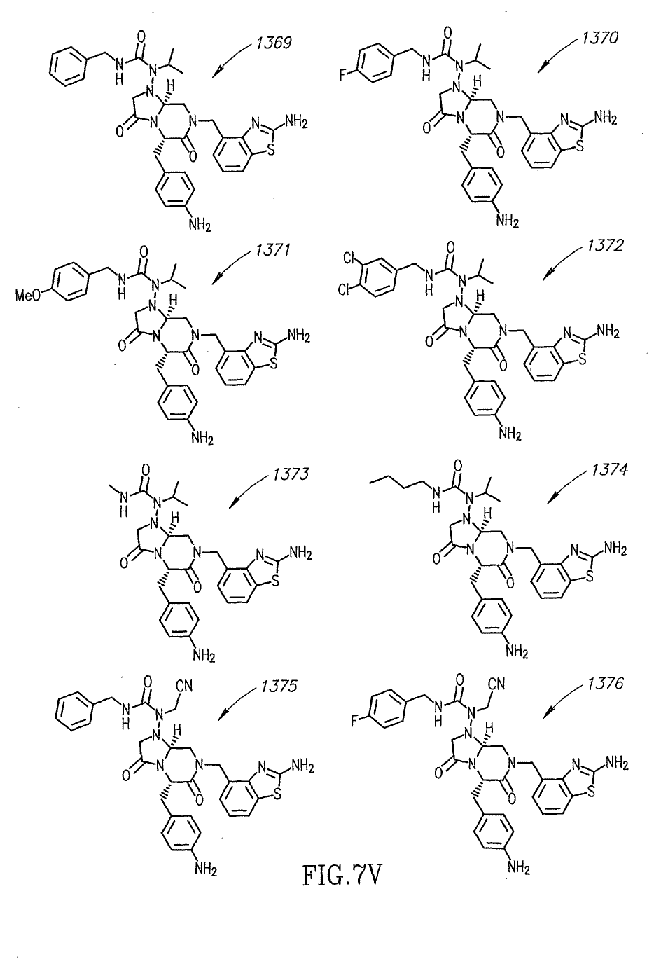

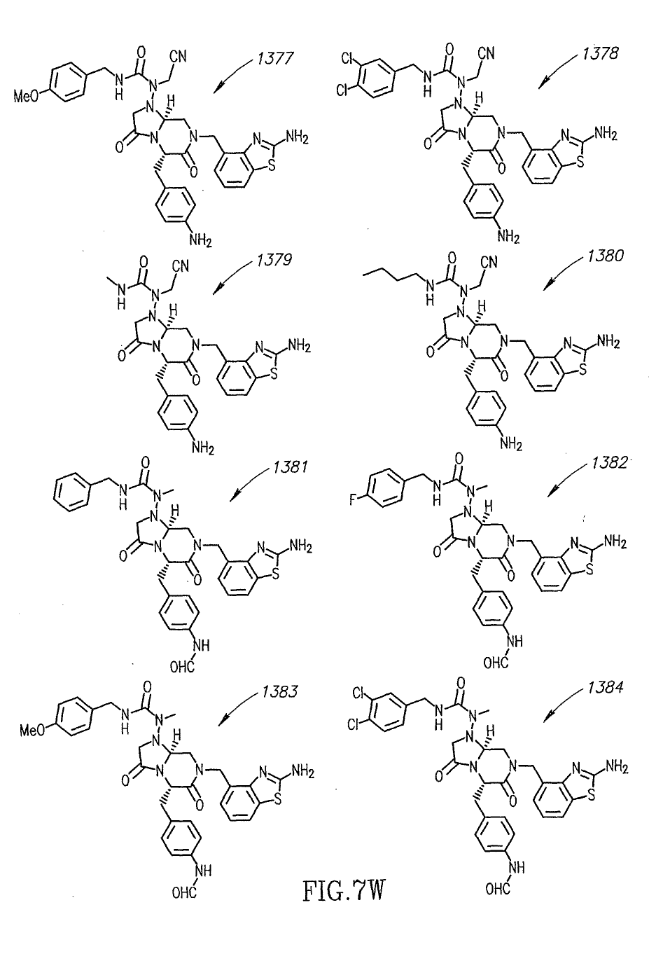

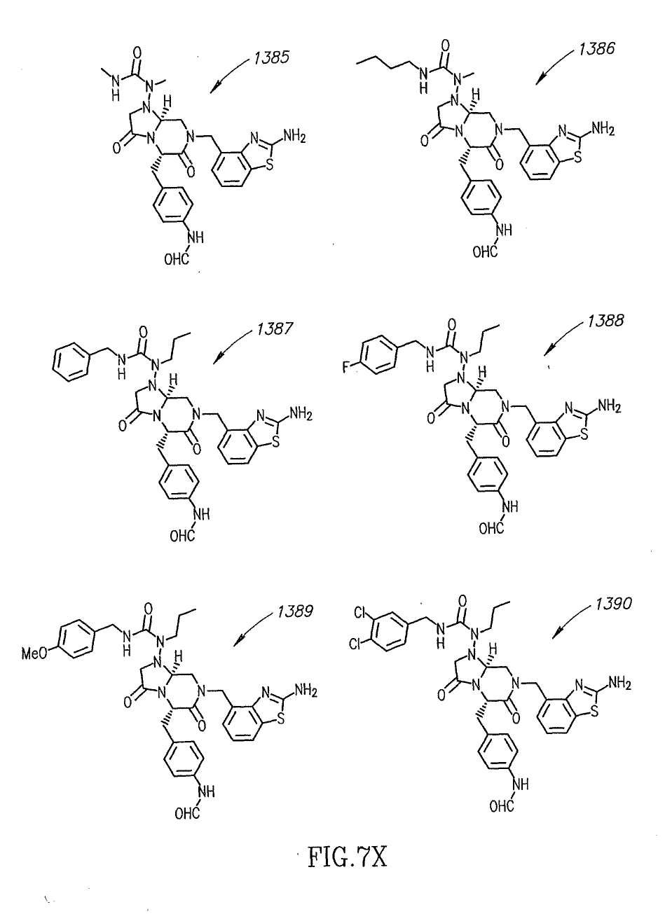

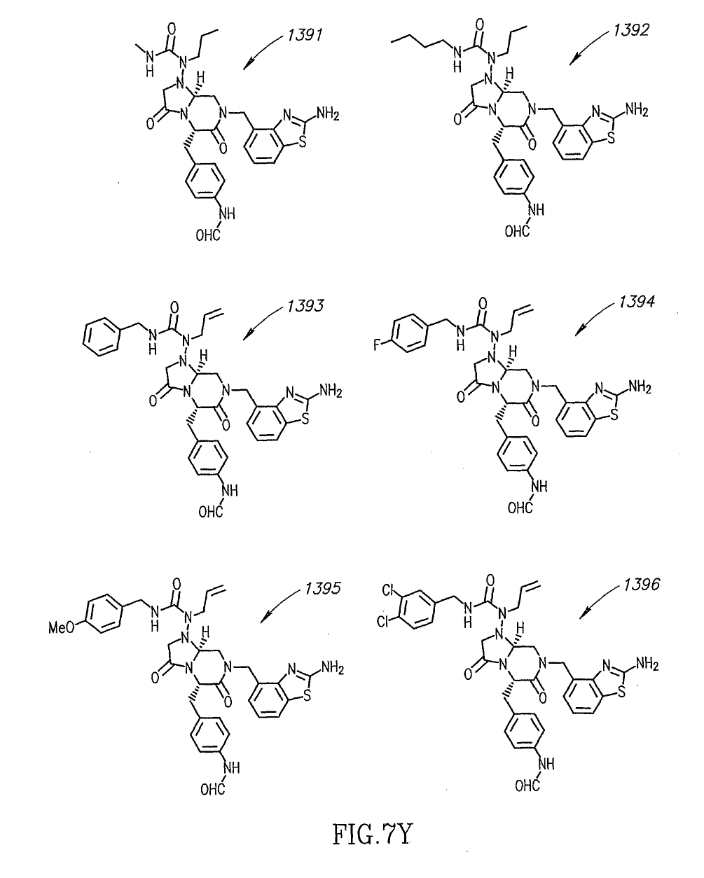

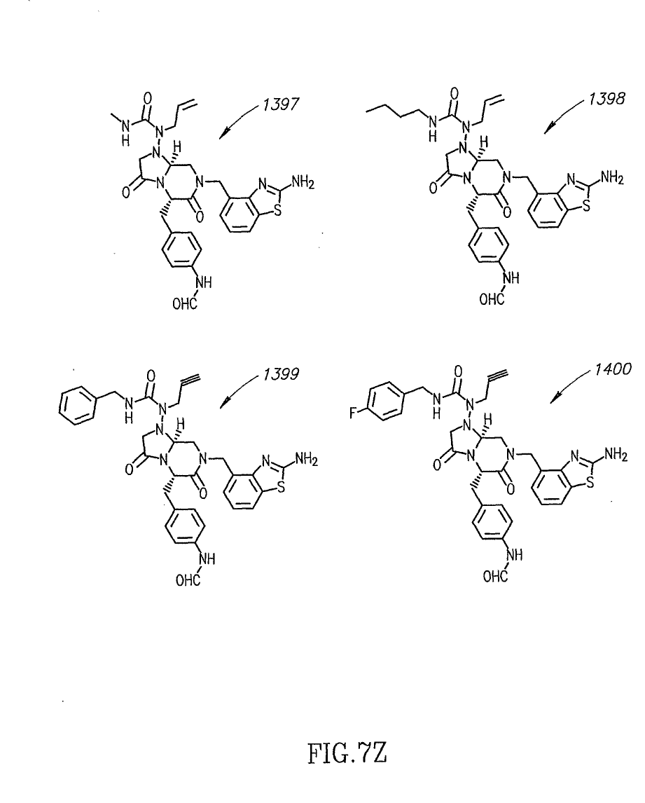

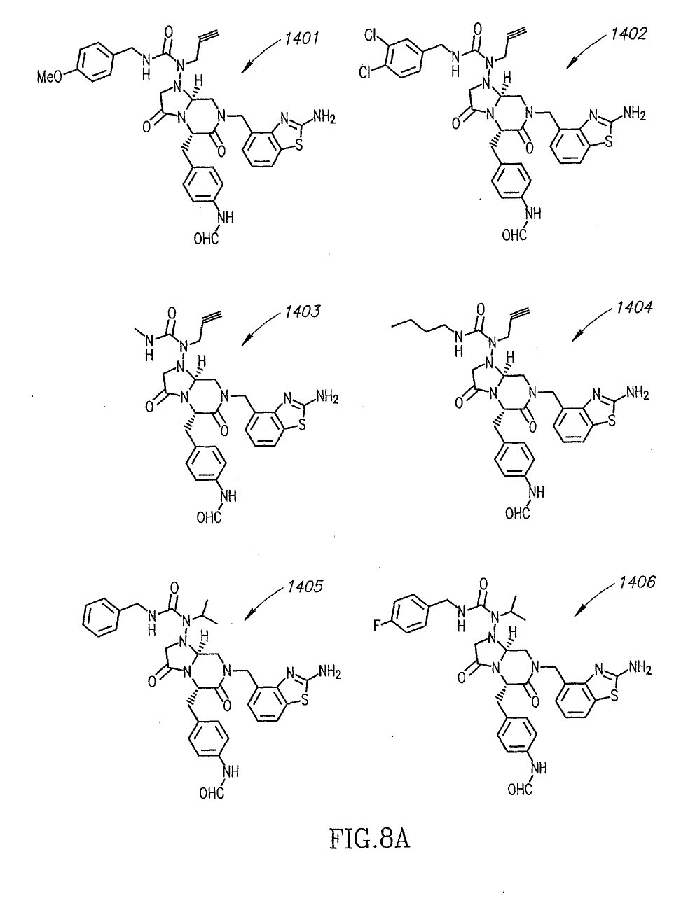

Similar synthesis was carried out to obtain Compounds 1201-2200 as shown in FIGS. 7-11.

Synthesis of Compound No. 2201

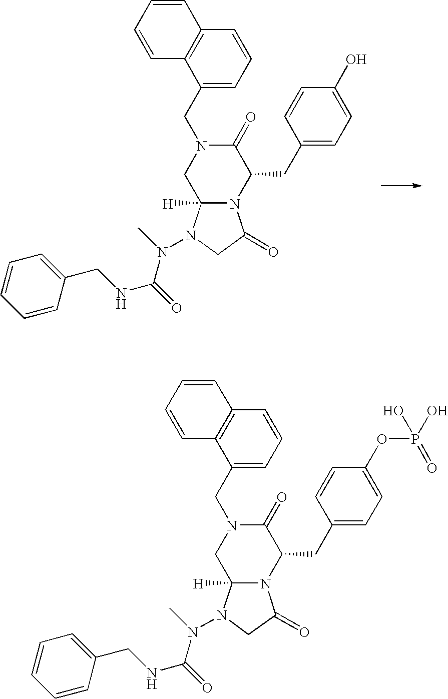

To the cooled (0δ) solution of Compound No. 61 (18 mg, 0.032 mmol) in THF (500 δL) were added Et₃N (13.4 μL, 0.096 mmol) and POCl₃ (14.9 μL, 0.160 mmol) and then the mixture was stirred till SM was disappeared on TLC (4 hr). The mixture was diluted with H₂O (1 mL) and then NaHCO₃ was added at 0δ to pH 8. After stirred overnight the mixture was acidified to pH 3 with 1N HCl followed by extraction with CHCl₃ (5 mL×3). The combined extracts were dried over Na₂SO₄, filtered and concentrated to afford pale yellow powder Compound No. 2201 (17.1 mg, 83%). TLC: Rf=0.45δSilica gel F254, CHCl₃:MeOH:EtOH:H₂O:AcOH:nBuOH=100:40:10:10:8:5δ; ¹H NMR (400 MHz, CDCl₃) δ 7.98 (1H, d, J=7.7 Hz), 7.83 (1H, m), 7.77 (1H, d, J=8.2 Hz), 7.51 (2H, m), 7.35 (1H, t, J=7.3 Hz), 7.24-6.93 (10H, m), 6.07 (1H, br s), 5.86 (3H, br s), 5.34 (1H, br d, J=15.0 Hz), 4.76 (2H, m), 4.11 (2H, br ABX, J=15.5, 5.3 Hz), 3.62 (2H, m), 3.47 and 3.31 (2H, br ABq, J=15.0 Hz), 3.22 (2H, br m), 3.02 (1H, br m), 2.77 (1H, br t, J=10.6 Hz), 2.56 (3H, s); ³¹P NMR (160.26 MHz, CDCl₃) δ-3.57.

Synthesis of Compound No. 2202

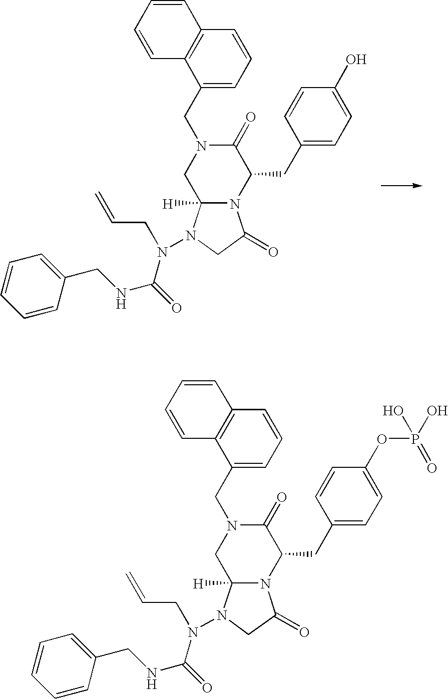

To the cooled (0δ) solution of Compound No. 71 (21 mg, 0.036 mmol) in THF (1.0 mL) were added Et₃N (14.9 μL, 0.107 mmol) and POCl₃ (16.6 μL, 0.178 mmol) and then the mixture was stirred till SM was disappeared on TLC (4 hr). The mixture was diluted with H₂O (1 mL) and then NaHCO₃ was added at 0δto pH 8. After stirred overnight, the mixture was acidified to pH 3 with 1N HCl followed by extraction with CHCl₃ (5 mL×3). The combined extracts were dried over Na₂SO₄, filtered and concentrated to afford pale yellow powder Compound No. 2202 (21.0 mg, 88%). TLC: Rf=0.53δSilica gel F254, CHCl₃:MeOH:EtOH:H₂O:AcOH:nBuOH=100:40:10:10:8:5δ.

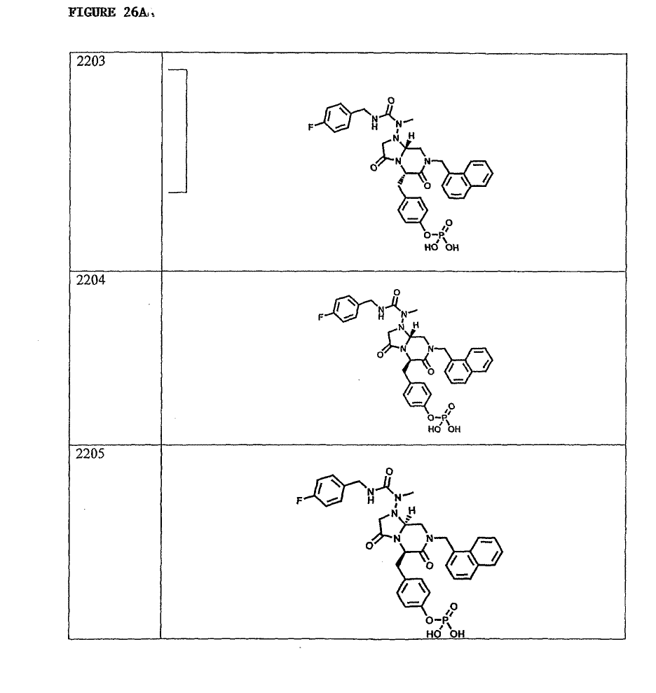

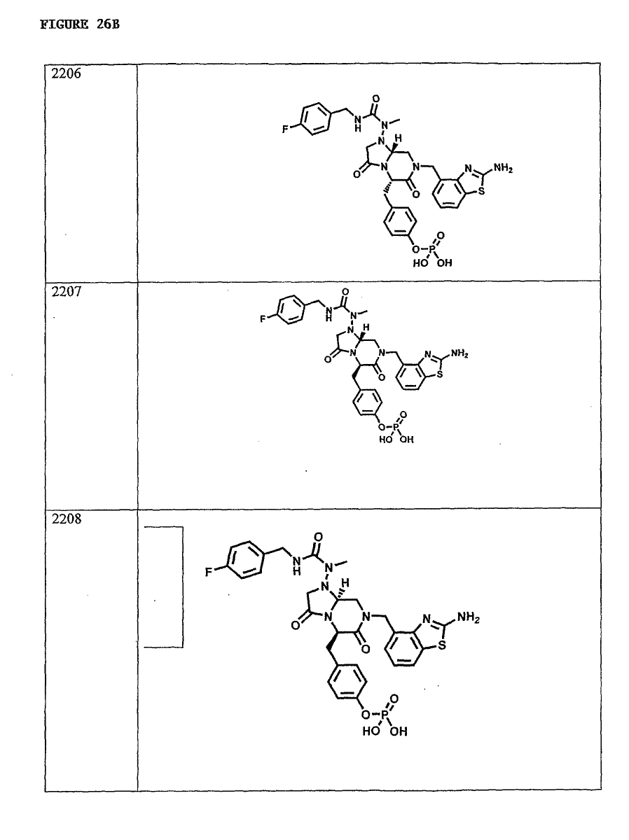

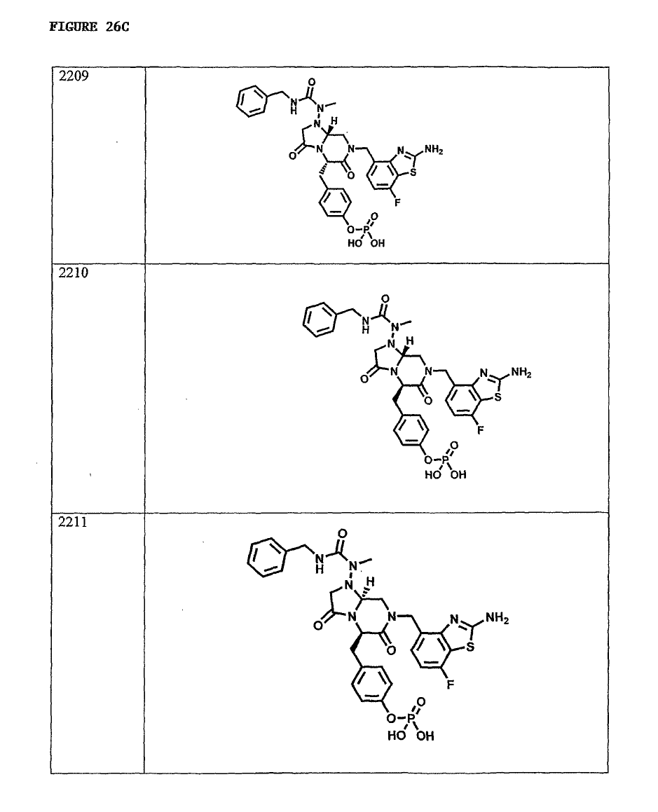

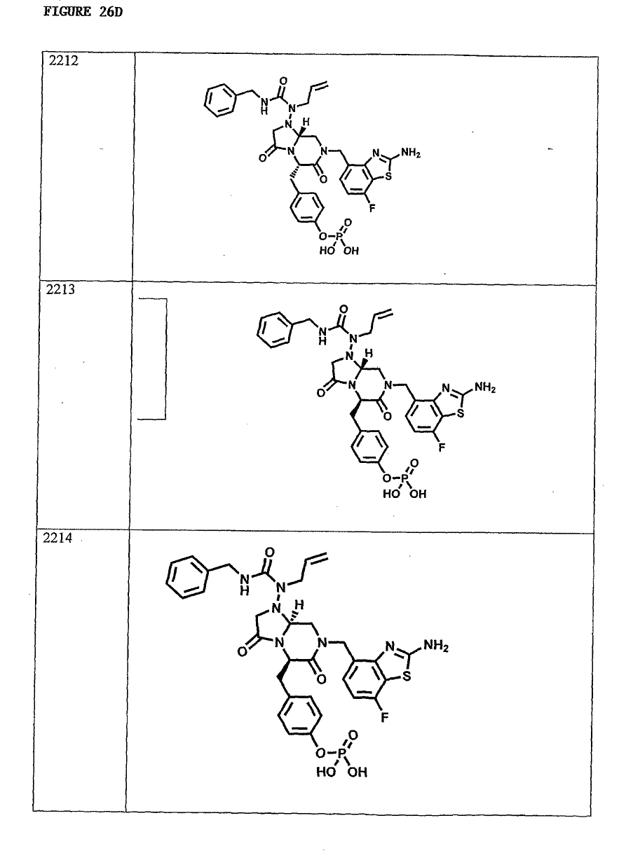

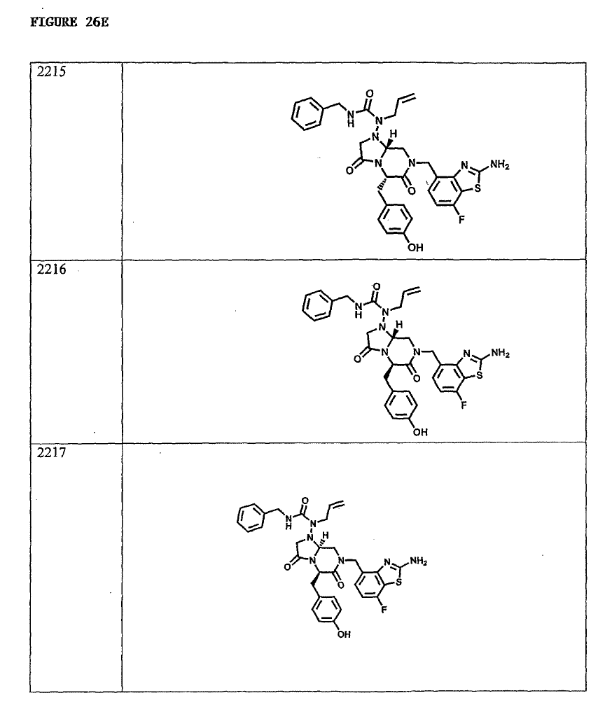

Similar synthesis was carried out to obtain Compounds 2203-2217 as shown in FIG. 26.

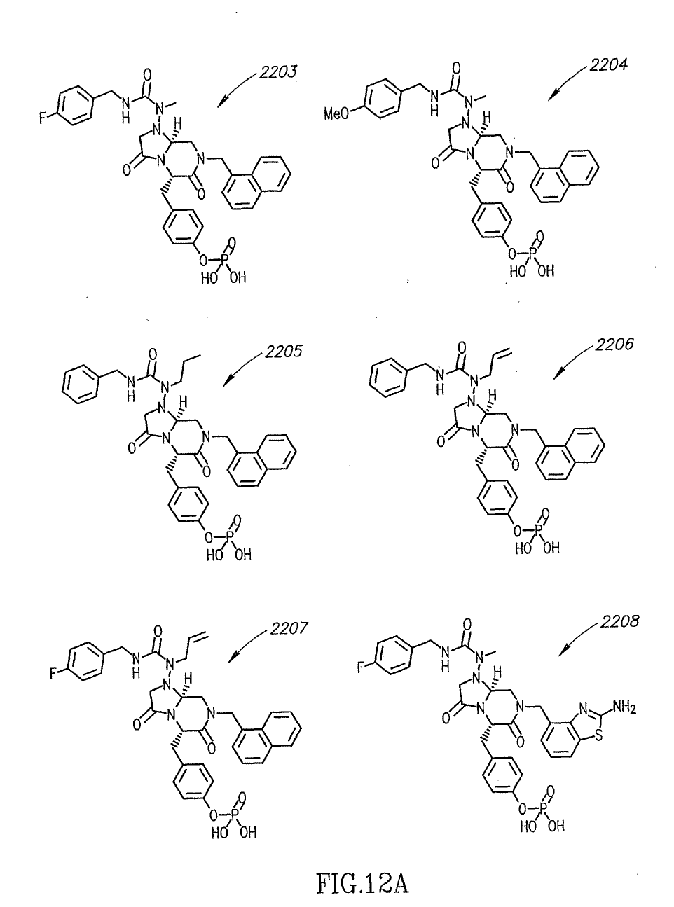

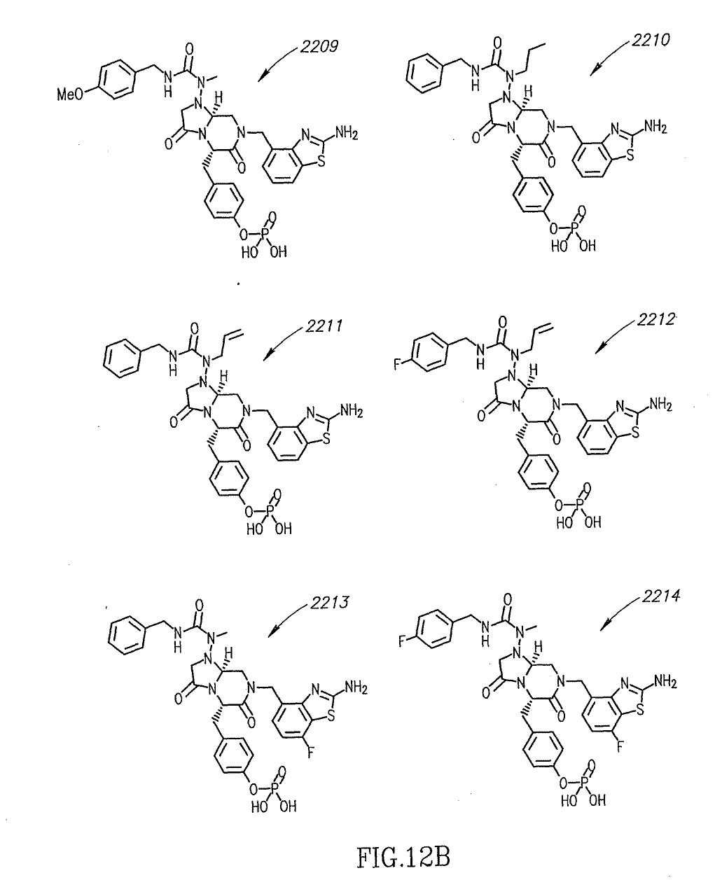

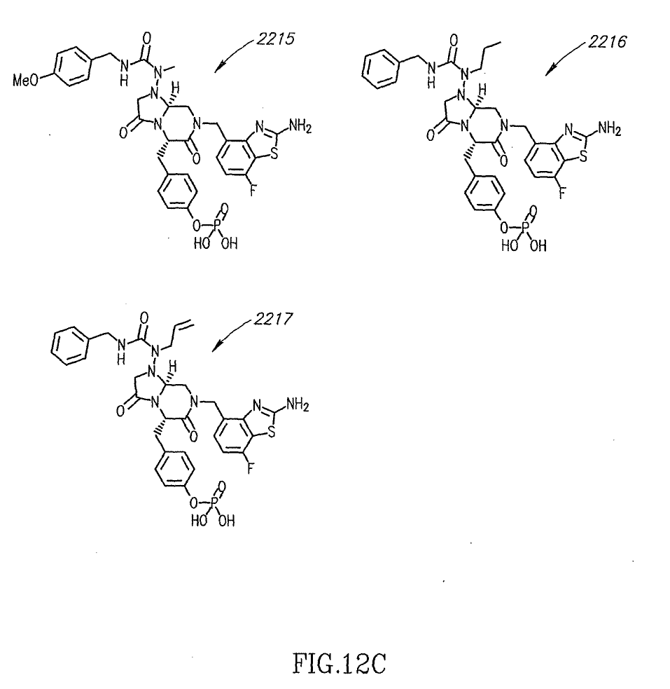

Diastereomeric and Enantiomeric stereo isomers of Compounds 2203-2217 were obtained and are shown FIG. 12.

Table 2 below shows the molecular weight (M.W.) and mass for compounds 1-2217.

TABLE 2

| Compound No. | M.W. | Mass |
|---|---|---|
| 1 | 533 | 534 |
| 2 | 551 | 552 |
| 3 | 563 | 564 |
| 4 | 602 | 603 |
| 5 | 457 | 458 |
| 6 | 561 | 562 |
| 7 | 579 | 580 |
| 8 | 591 | 592 |
| 9 | 630 | 631 |
| 10 | 485 | 486 |
| 11 | 559 | 560 |
| 12 | 577 | 578 |
| 13 | 589 | 590 |
| 14 | 628 | 629 |
| 15 | 483 | 484 |
| 16 | 557 | 558 |
| 17 | 575 | 576 |
| 18 | 587 | 588 |
| 19 | 626 | 627 |
| 20 | 481 | 482 |
| 21 | 561 | 562 |
| 22 | 579 | 580 |
| 23 | 591 | 592 |
| 24 | 630 | 631 |
| 25 | 485 | 486 |
| 26 | 558 | 559 |
| 27 | 576 | 577 |
| 28 | 588 | 589 |
| 29 | 627 | 628 |
| 30 | 482 | 483 |
| 31 | 547 | 548 |
| 32 | 565 | 566 |
| 33 | 577 | 578 |
| 34 | 616 | 617 |
| 35 | 471 | 472 |
| 36 | 575 | 576 |
| 37 | 593 | 594 |
| 38 | 605 | 606 |
| 39 | 644 | 645 |
| 40 | 499 | 500 |
| 41 | 573 | 574 |
| 42 | 591 | 592 |
| 43 | 603 | 604 |
| 44 | 642 | 643 |
| 45 | 497 | 498 |
| 46 | 571 | 572 |
| 47 | 589 | 590 |
| 48 | 601 | 602 |
| 49 | 640 | 641 |
| 50 | 495 | 496 |
| 51 | 575 | 576 |
| 52 | 593 | 594 |
| 53 | 605 | 606 |
| 54 | 644 | 645 |
| 55 | 499 | 500 |
| 56 | 572 | 573 |
| 57 | 590 | 591 |
| 58 | 602 | 603 |
| 59 | 641 | 642 |
| 60 | 496 | 497 |
| 61 | 563 | 564 |
| 62 | 581 | 582 |
| 63 | 593 | 594 |
| 64 | 632 | 633 |
| 65 | 487 | 488 |
| 66 | 591 | 592 |
| 67 | 609 | 610 |
| 68 | 621 | 622 |
| 69 | 660 | 661 |
| 70 | 515 | 516 |
| 71 | 589 | 590 |
| 72 | 607 | 608 |
| 73 | 619 | 620 |
| 74 | 658 | 659 |
| 75 | 513 | 514 |
| 76 | 587 | 588 |
| 77 | 605 | 606 |
| 78 | 617 | 618 |
| 79 | 656 | 657 |
| 80 | 511 | 512 |
| 81 | 591 | 592 |
| 82 | 609 | 610 |
| 83 | 621 | 622 |
| 84 | 660 | 661 |
| 85 | 515 | 516 |
| 86 | 588 | 589 |
| 87 | 606 | 607 |
| 88 | 618 | 619 |
| 89 | 657 | 658 |
| 90 | 512 | 513 |
| 91 | 563 | 564 |
| 92 | 581 | 582 |
| 93 | 609 | 610 |
| 94 | 648 | 649 |
| 95 | 503 | 504 |
| 96 | 607 | 608 |
| 97 | 625 | 626 |
| 98 | 637 | 638 |
| 99 | 676 | 677 |
| 100 | 531 | 532 |
| 101 | 605 | 606 |
| 102 | 623 | 624 |
| 103 | 635 | 636 |
| 104 | 674 | 675 |
| 105 | 529 | 530 |
| 106 | 603 | 604 |
| 107 | 621 | 622 |
| 108 | 633 | 634 |
| 109 | 672 | 673 |
| 110 | 527 | 528 |
| 111 | 607 | 608 |
| 112 | 625 | 626 |
| 113 | 637 | 638 |
| 114 | 676 | 677 |
| 115 | 531 | 532 |
| 116 | 604 | 605 |
| 117 | 622 | 623 |
| 118 | 634 | 635 |
| 119 | 673 | 674 |
| 120 | 528 | 529 |
| 121 | 562 | 563 |
| 122 | 580 | 581 |
| 123 | 592 | 593 |
| 124 | 631 | 632 |
| 125 | 486 | 487 |
| 126 | 590 | 591 |
| 127 | 608 | 609 |
| 128 | 620 | 621 |
| 129 | 659 | 660 |
| 130 | 514 | 515 |
| 131 | 588 | 589 |
| 132 | 606 | 607 |
| 133 | 618 | 619 |
| 134 | 657 | 658 |
| 135 | 512 | 513 |
| 136 | 586 | 587 |
| 137 | 604 | 605 |
| 138 | 616 | 617 |
| 139 | 655 | 656 |
| 140 | 510 | 511 |
| 141 | 590 | 591 |
| 142 | 608 | 609 |
| 143 | 620 | 621 |
| 144 | 659 | 660 |
| 145 | 514 | 515 |
| 146 | 587 | 588 |
| 147 | 605 | 606 |
| 148 | 617 | 618 |
| 149 | 656 | 657 |
| 150 | 511 | 512 |
| 151 | 590 | 591 |
| 152 | 608 | 609 |
| 153 | 620 | 621 |
| 154 | 659 | 660 |
| 155 | 514 | 515 |
| 156 | 618 | 619 |
| 157 | 636 | 637 |
| 158 | 648 | 649 |
| 159 | 687 | 688 |
| 160 | 542 | 543 |
| 161 | 616 | 617 |
| 162 | 634 | 635 |
| 163 | 646 | 647 |
| 164 | 685 | 686 |
| 165 | 540 | 541 |
| 166 | 614 | 615 |
| 167 | 632 | 633 |
| 168 | 644 | 645 |
| 169 | 683 | 684 |
| 170 | 538 | 539 |
| 171 | 618 | 619 |
| 172 | 636 | 637 |
| 173 | 648 | 649 |
| 174 | 687 | 688 |
| 175 | 542 | 543 |
| 176 | 615 | 616 |
| 177 | 633 | 634 |
| 178 | 645 | 646 |
| 179 | 684 | 685 |
| 180 | 539 | 540 |
| 181 | 666 | 667 |
| 182 | 684 | 685 |
| 183 | 696 | 697 |
| 184 | 735 | 736 |
| 185 | 590 | 591 |
| 186 | 694 | 695 |
| 187 | 712 | 713 |
| 188 | 724 | 725 |
| 189 | 763 | 764 |
| 190 | 618 | 619 |
| 191 | 692 | 693 |
| 192 | 710 | 711 |
| 193 | 722 | 723 |
| 194 | 761 | 762 |
| 195 | 616 | 617 |
| 196 | 690 | 691 |
| 197 | 708 | 709 |
| 198 | 720 | 721 |
| 199 | 759 | 760 |
| 200 | 614 | 615 |
| 201 | 694 | 695 |
| 202 | 712 | 713 |
| 203 | 724 | 725 |
| 204 | 763 | 764 |
| 205 | 618 | 619 |
| 206 | 691 | 692 |
| 207 | 709 | 710 |
| 208 | 721 | 722 |
| 209 | 760 | 761 |
| 210 | 615 | 616 |
| 211 | 696 | 697 |
| 212 | 714 | 715 |
| 213 | 726 | 727 |
| 214 | 765 | 766 |
| 215 | 620 | 621 |
| 216 | 724 | 725 |
| 217 | 742 | 743 |
| 218 | 754 | 755 |
| 219 | 793 | 794 |
| 220 | 648 | 649 |
| 221 | 722 | 723 |
| 222 | 740 | 741 |
| 223 | 752 | 753 |
| 224 | 791 | 792 |
| 225 | 646 | 647 |
| 226 | 720 | 721 |
| 227 | 738 | 739 |
| 228 | 750 | 751 |
| 229 | 789 | 790 |
| 230 | 644 | 645 |
| 231 | 724 | 725 |
| 232 | 742 | 743 |
| 233 | 754 | 755 |
| 234 | 793 | 794 |
| 235 | 648 | 649 |
| 236 | 721 | 722 |
| 237 | 739 | 740 |
| 238 | 751 | 752 |
| 239 | 790 | 791 |
| 240 | 645 | 646 |
| 241 | 590 | 591 |
| 242 | 608 | 609 |
| 243 | 620 | 621 |
| 244 | 659 | 660 |
| 245 | 514 | 515 |
| 246 | 618 | 619 |
| 247 | 636 | 637 |
| 248 | 648 | 649 |
| 249 | 687 | 688 |
| 250 | 542 | 543 |
| 251 | 616 | 617 |
| 252 | 634 | 635 |
| 253 | 646 | 647 |
| 254 | 685 | 686 |
| 255 | 540 | 541 |
| 256 | 614 | 615 |
| 257 | 632 | 633 |
| 258 | 644 | 645 |
| 259 | 683 | 684 |
| 260 | 538 | 539 |
| 261 | 618 | 619 |
| 262 | 636 | 637 |
| 263 | 648 | 649 |
| 264 | 687 | 688 |
| 265 | 542 | 543 |
| 266 | 615 | 616 |
| 267 | 633 | 634 |
| 268 | 645 | 646 |
| 269 | 684 | 685 |
| 270 | 539 | 540 |
| 271 | 592 | 593 |
| 272 | 610 | 611 |
| 273 | 622 | 623 |
| 274 | 661 | 662 |
| 275 | 516 | 517 |
| 276 | 620 | 621 |
| 277 | 638 | 639 |
| 278 | 650 | 651 |
| 279 | 689 | 690 |
| 280 | 544 | 545 |
| 281 | 618 | 619 |
| 282 | 636 | 637 |
| 283 | 648 | 649 |
| 284 | 687 | 688 |
| 285 | 542 | 543 |
| 286 | 616 | 617 |
| 287 | 634 | 635 |
| 288 | 646 | 647 |
| 289 | 685 | 686 |
| 290 | 540 | 541 |
| 291 | 620 | 621 |
| 292 | 638 | 639 |
| 293 | 650 | 651 |
| 294 | 689 | 690 |
| 295 | 544 | 545 |
| 296 | 617 | 618 |
| 297 | 635 | 636 |
| 298 | 647 | 648 |
| 299 | 686 | 687 |
| 300 | 541 | 542 |
| 301 | 577 | 578 |
| 302 | 595 | 596 |
| 303 | 607 | 608 |
| 304 | 646 | 647 |
| 305 | 501 | 502 |
| 306 | 605 | 606 |
| 307 | 623 | 624 |
| 308 | 635 | 636 |
| 309 | 674 | 675 |
| 310 | 529 | 530 |
| 311 | 603 | 604 |
| 312 | 621 | 622 |
| 313 | 633 | 634 |
| 314 | 672 | 673 |
| 315 | 527 | 528 |
| 316 | 601 | 602 |
| 317 | 619 | 620 |
| 318 | 631 | 632 |
| 319 | 670 | 671 |
| 320 | 525 | 526 |
| 321 | 605 | 606 |
| 322 | 623 | 624 |
| 323 | 635 | 636 |
| 324 | 674 | 675 |
| 325 | 529 | 530 |
| 326 | 602 | 603 |
| 327 | 620 | 621 |
| 328 | 632 | 633 |
| 329 | 671 | 672 |
| 330 | 526 | 527 |
| 331 | 635 | 636 |
| 332 | 653 | 654 |
| 333 | 665 | 666 |
| 334 | 704 | 705 |
| 335 | 559 | 560 |
| 336 | 663 | 664 |
| 337 | 681 | 682 |
| 338 | 693 | 694 |
| 339 | 732 | 733 |
| 340 | 587 | 588 |
| 341 | 661 | 662 |
| 342 | 679 | 680 |
| 343 | 691 | 692 |
| 344 | 730 | 731 |
| 345 | 585 | 586 |
| 346 | 659 | 660 |
| 347 | 677 | 678 |
| 348 | 689 | 690 |
| 349 | 728 | 729 |
| 350 | 583 | 584 |
| 351 | 663 | 664 |
| 352 | 681 | 682 |
| 353 | 693 | 694 |
| 354 | 732 | 733 |
| 355 | 587 | 588 |
| 356 | 660 | 661 |
| 357 | 678 | 679 |
| 358 | 690 | 691 |
| 359 | 729 | 730 |
| 360 | 584 | 585 |
| 361 | 716 | 717 |
| 362 | 734 | 735 |
| 363 | 746 | 747 |
| 364 | 785 | 786 |
| 365 | 640 | 641 |
| 366 | 744 | 745 |
| 367 | 762 | 763 |
| 368 | 774 | 775 |
| 369 | 813 | 814 |
| 370 | 668 | 669 |
| 371 | 742 | 743 |
| 372 | 760 | 761 |
| 373 | 772 | 773 |
| 374 | 811 | 812 |
| 375 | 666 | 667 |
| 376 | 740 | 741 |
| 377 | 758 | 759 |
| 378 | 770 | 771 |
| 379 | 809 | 810 |
| 380 | 664 | 665 |
| 381 | 744 | 745 |
| 382 | 762 | 763 |
| 383 | 774 | 775 |
| 384 | 813 | 814 |
| 385 | 668 | 669 |
| 386 | 741 | 742 |
| 387 | 759 | 760 |
| 388 | 771 | 772 |
| 389 | 810 | 811 |
| 390 | 665 | 666 |
| 391 | 565 | 566 |
| 392 | 583 | 584 |
| 393 | 595 | 596 |
| 394 | 634 | 635 |
| 395 | 489 | 490 |
| 396 | 593 | 594 |
| 397 | 611 | 612 |
| 398 | 623 | 624 |
| 399 | 662 | 663 |
| 400 | 517 | 518 |
| 401 | 591 | 592 |
| 402 | 609 | 610 |
| 403 | 621 | 622 |
| 404 | 660 | 661 |
| 405 | 515 | 516 |
| 406 | 589 | 590 |
| 407 | 607 | 608 |
| 408 | 619 | 620 |
| 409 | 658 | 659 |
| 410 | 513 | 514 |
| 411 | 593 | 594 |
| 412 | 611 | 612 |
| 413 | 623 | 624 |
| 414 | 662 | 663 |
| 415 | 517 | 518 |
| 416 | 590 | 591 |
| 417 | 608 | 609 |
| 418 | 620 | 621 |
| 419 | 659 | 660 |
| 420 | 514 | 515 |
| 421 | 578 | 579 |
| 422 | 596 | 597 |
| 423 | 608 | 609 |
| 424 | 647 | 648 |
| 425 | 502 | 503 |
| 426 | 606 | 607 |
| 427 | 624 | 625 |
| 428 | 636 | 637 |
| 429 | 675 | 676 |
| 430 | 530 | 531 |
| 431 | 604 | 605 |
| 432 | 622 | 623 |
| 433 | 634 | 635 |
| 434 | 673 | 674 |
| 435 | 528 | 529 |
| 436 | 602 | 603 |
| 437 | 620 | 621 |
| 438 | 632 | 633 |
| 439 | 671 | 672 |
| 440 | 526 | 527 |
| 441 | 606 | 607 |
| 442 | 624 | 625 |
| 443 | 636 | 637 |
| 444 | 675 | 676 |
| 445 | 530 | 531 |
| 446 | 603 | 604 |
| 447 | 621 | 622 |
| 448 | 633 | 634 |
| 449 | 672 | 673 |
| 450 | 527 | 528 |
| 451 | 634 | 635 |
| 452 | 652 | 653 |
| 453 | 664 | 665 |
| 454 | 703 | 704 |
| 455 | 558 | 559 |
| 456 | 662 | 663 |
| 457 | 680 | 681 |
| 458 | 692 | 693 |
| 459 | 731 | 732 |
| 460 | 586 | 587 |
| 461 | 660 | 661 |
| 462 | 678 | 679 |
| 463 | 690 | 691 |
| 464 | 729 | 730 |
| 465 | 584 | 585 |
| 466 | 658 | 659 |
| 467 | 676 | 677 |
| 468 | 688 | 689 |
| 469 | 727 | 728 |
| 470 | 582 | 583 |
| 471 | 662 | 663 |
| 472 | 680 | 681 |
| 473 | 692 | 693 |
| 474 | 731 | 732 |
| 475 | 586 | 587 |
| 476 | 659 | 660 |
| 477 | 677 | 678 |
| 478 | 689 | 690 |
| 479 | 728 | 729 |
| 480 | 583 | 584 |
| 481 | 677 | 678 |
| 482 | 695 | 696 |
| 483 | 707 | 708 |
| 484 | 746 | 747 |
| 485 | 601 | 602 |
| 486 | 705 | 706 |
| 487 | 723 | 724 |
| 488 | 735 | 736 |
| 489 | 774 | 775 |
| 490 | 629 | 630 |
| 491 | 703 | 704 |
| 492 | 721 | 722 |
| 493 | 733 | 734 |
| 494 | 772 | 773 |
| 495 | 627 | 628 |
| 496 | 701 | 702 |
| 497 | 719 | 720 |
| 498 | 731 | 732 |
| 499 | 770 | 771 |
| 500 | 625 | 626 |
| 501 | 705 | 706 |
| 502 | 723 | 724 |
| 503 | 735 | 736 |
| 504 | 774 | 775 |
| 505 | 629 | 630 |
| 506 | 702 | 703 |
| 507 | 720 | 721 |
| 508 | 732 | 733 |
| 509 | 771 | 772 |
| 510 | 626 | 627 |
| 511 | 607 | 608 |
| 512 | 625 | 626 |
| 513 | 637 | 638 |
| 514 | 676 | 677 |
| 515 | 531 | 532 |
| 516 | 635 | 636 |
| 517 | 653 | 654 |
| 518 | 665 | 666 |
| 519 | 704 | 705 |
| 520 | 559 | 560 |
| 521 | 633 | 634 |
| 522 | 651 | 652 |
| 523 | 663 | 664 |
| 524 | 702 | 703 |
| 525 | 557 | 558 |
| 526 | 631 | 632 |
| 527 | 649 | 650 |
| 528 | 661 | 662 |
| 529 | 700 | 701 |
| 530 | 555 | 556 |
| 531 | 635 | 636 |
| 532 | 653 | 654 |
| 533 | 665 | 666 |
| 534 | 704 | 705 |
| 535 | 559 | 560 |
| 536 | 632 | 633 |
| 537 | 650 | 651 |
| 538 | 662 | 663 |
| 539 | 701 | 702 |
| 540 | 556 | 557 |
| 541 | 640 | 641 |
| 542 | 658 | 659 |
| 543 | 670 | 671 |
| 544 | 709 | 710 |
| 545 | 564 | 565 |
| 546 | 668 | 669 |
| 547 | 686 | 687 |
| 548 | 698 | 699 |
| 549 | 737 | 738 |
| 550 | 592 | 593 |
| 551 | 666 | 667 |
| 552 | 684 | 685 |
| 553 | 696 | 697 |
| 554 | 735 | 736 |
| 555 | 590 | 591 |
| 556 | 664 | 665 |
| 557 | 682 | 683 |
| 558 | 694 | 695 |
| 559 | 733 | 734 |
| 560 | 588 | 589 |
| 561 | 668 | 669 |
| 562 | 686 | 687 |
| 563 | 698 | 699 |
| 564 | 737 | 738 |
| 565 | 592 | 593 |
| 566 | 665 | 666 |
| 567 | 683 | 684 |
| 568 | 695 | 696 |
| 569 | 734 | 735 |
| 570 | 589 | 590 |
| 571 | 587 | 588 |
| 572 | 605 | 606 |
| 573 | 617 | 618 |
| 574 | 656 | 657 |
| 575 | 511 | 512 |
| 576 | 615 | 616 |
| 577 | 633 | 634 |
| 578 | 645 | 646 |
| 579 | 684 | 685 |
| 580 | 539 | 540 |
| 581 | 613 | 614 |
| 582 | 631 | 632 |
| 583 | 643 | 644 |
| 584 | 682 | 683 |
| 585 | 537 | 538 |
| 591 | 615 | 616 |
| 592 | 633 | 634 |
| 593 | 645 | 646 |
| 594 | 684 | 685 |
| 595 | 539 | 540 |
| 586 | 611 | 612 |
| 587 | 629 | 630 |
| 588 | 641 | 642 |
| 589 | 680 | 681 |
| 590 | 535 | 536 |
| 596 | 612 | 613 |
| 597 | 630 | 631 |
| 598 | 642 | 643 |
| 599 | 681 | 682 |
| 600 | 536 | 537 |
| 601 | 551 | 552 |
| 602 | 579 | 580 |
| 603 | 577 | 578 |
| 604 | 565 | 566 |
| 605 | 593 | 594 |
| 606 | 591 | 592 |
| 607 | 581 | 582 |
| 608 | 609 | 610 |
| 609 | 607 | 608 |
| 610 | 497 | 498 |
| 611 | 525 | 526 |
| 612 | 523 | 524 |
| 613 | 511 | 512 |
| 614 | 539 | 540 |
| 615 | 537 | 538 |
| 616 | 527 | 528 |
| 617 | 555 | 556 |
| 618 | 553 | 554 |
| 619 | 513 | 514 |
| 620 | 541 | 542 |
| 621 | 539 | 540 |
| 622 | 527 | 528 |
| 623 | 555 | 556 |
| 624 | 553 | 554 |
| 625 | 543 | 544 |
| 626 | 571 | 572 |
| 627 | 569 | 570 |
| 628 | 483 | 484 |
| 629 | 511 | 512 |
| 630 | 509 | 510 |
| 631 | 497 | 498 |
| 632 | 525 | 526 |
| 633 | 523 | 524 |
| 634 | 513 | 514 |
| 635 | 541 | 542 |
| 636 | 539 | 540 |
| 637 | 518 | 519 |
| 638 | 546 | 547 |
| 639 | 544 | 545 |
| 640 | 532 | 533 |
| 641 | 560 | 561 |
| 642 | 558 | 559 |
| 643 | 548 | 549 |
| 644 | 576 | 577 |
| 645 | 574 | 575 |
| 646 | 553 | 554 |
| 647 | 581 | 582 |
| 648 | 579 | 580 |
| 649 | 567 | 568 |
| 650 | 595 | 596 |
| 651 | 593 | 594 |
| 652 | 583 | 584 |
| 653 | 611 | 612 |
| 654 | 609 | 610 |
| 655 | 553 | 554 |
| 656 | 581 | 582 |
| 657 | 579 | 580 |
| 658 | 567 | 568 |
| 659 | 595 | 596 |
| 660 | 593 | 594 |
| 661 | 583 | 584 |
| 662 | 611 | 612 |
| 663 | 609 | 610 |
| 664 | 563 | 564 |
| 665 | 591 | 592 |
| 666 | 589 | 590 |
| 667 | 577 | 578 |
| 668 | 605 | 606 |
| 669 | 603 | 604 |
| 670 | 593 | 594 |
| 671 | 621 | 622 |
| 672 | 619 | 620 |
| 673 | 545 | 546 |
| 674 | 573 | 574 |
| 675 | 571 | 572 |
| 676 | 559 | 560 |
| 677 | 587 | 588 |
| 678 | 585 | 586 |
| 679 | 575 | 576 |
| 680 | 603 | 604 |
| 681 | 601 | 602 |
| 682 | 518 | 519 |
| 683 | 546 | 547 |
| 684 | 544 | 545 |
| 685 | 532 | 533 |
| 686 | 560 | 561 |
| 687 | 558 | 559 |
| 688 | 548 | 549 |
| 689 | 576 | 577 |
| 690 | 574 | 575 |
| 691 | 497 | 498 |
| 692 | 525 | 526 |
| 693 | 523 | 524 |
| 694 | 511 | 512 |
| 695 | 539 | 540 |
| 696 | 537 | 538 |
| 697 | 527 | 528 |
| 698 | 555 | 556 |
| 699 | 553 | 554 |
| 700 | 497 | 498 |
| 701 | 525 | 526 |
| 702 | 523 | 524 |
| 703 | 511 | 512 |
| 704 | 539 | 540 |
| 705 | 537 | 538 |
| 706 | 527 | 528 |
| 707 | 555 | 556 |
| 708 | 553 | 554 |
| 709 | 497 | 498 |
| 710 | 525 | 526 |
| 711 | 523 | 524 |
| 712 | 511 | 512 |
| 713 | 539 | 540 |
| 714 | 537 | 538 |
| 715 | 527 | 528 |
| 716 | 555 | 556 |
| 717 | 553 | 554 |
| 718 | 541 | 542 |
| 719 | 569 | 570 |
| 720 | 567 | 568 |
| 721 | 555 | 556 |
| 722 | 583 | 584 |
| 723 | 581 | 582 |
| 724 | 571 | 572 |
| 725 | 599 | 600 |
| 726 | 597 | 598 |
| 727 | 554 | 555 |
| 728 | 582 | 583 |
| 729 | 580 | 581 |
| 730 | 568 | 569 |
| 731 | 596 | 597 |
| 732 | 594 | 595 |
| 733 | 584 | 585 |
| 734 | 612 | 613 |
| 735 | 610 | 611 |
| 736 | 554 | 555 |
| 737 | 582 | 583 |
| 738 | 580 | 581 |
| 739 | 568 | 569 |
| 740 | 596 | 597 |
| 741 | 594 | 595 |
| 742 | 584 | 585 |
| 743 | 612 | 613 |
| 744 | 610 | 611 |
| 745 | 554 | 555 |
| 746 | 582 | 583 |
| 747 | 580 | 581 |
| 748 | 568 | 569 |
| 749 | 596 | 597 |
| 750 | 594 | 595 |
| 751 | 584 | 585 |
| 752 | 612 | 613 |
| 753 | 610 | 611 |
| 754 | 561 | 562 |
| 755 | 589 | 590 |
| 756 | 587 | 588 |
| 757 | 575 | 576 |
| 758 | 603 | 604 |
| 759 | 601 | 602 |
| 760 | 591 | 592 |
| 761 | 619 | 620 |
| 762 | 617 | 618 |
| 763 | 562 | 563 |
| 764 | 590 | 591 |
| 765 | 588 | 589 |
| 766 | 576 | 577 |
| 767 | 604 | 605 |
| 768 | 602 | 603 |
| 769 | 592 | 593 |
| 770 | 620 | 621 |
| 771 | 618 | 619 |
| 772 | 568 | 569 |
| 773 | 596 | 597 |
| 774 | 594 | 595 |
| 775 | 582 | 583 |
| 776 | 610 | 611 |
| 777 | 608 | 609 |
| 778 | 598 | 599 |
| 779 | 626 | 627 |
| 780 | 624 | 625 |
| 781 | 603 | 604 |
| 782 | 631 | 632 |
| 783 | 629 | 630 |
| 784 | 617 | 618 |
| 785 | 645 | 646 |
| 791 | 555 | 556 |
| 792 | 553 | 554 |
| 793 | 541 | 542 |
| 794 | 569 | 570 |
| 795 | 567 | 568 |
| 786 | 643 | 644 |
| 787 | 633 | 634 |
| 788 | 661 | 662 |
| 789 | 659 | 660 |
| 790 | 527 | 528 |
| 796 | 557 | 558 |
| 797 | 585 | 586 |
| 798 | 583 | 584 |
| 799 | 544 | 545 |
| 800 | 572 | 573 |
| 801 | 570 | 571 |
| 802 | 558 | 559 |
| 803 | 586 | 587 |
| 804 | 584 | 585 |
| 805 | 574 | 575 |
| 806 | 602 | 603 |
| 807 | 600 | 601 |
| 808 | 526 | 527 |
| 809 | 554 | 555 |
| 810 | 552 | 553 |
| 811 | 540 | 541 |
| 812 | 568 | 569 |
| 813 | 566 | 567 |
| 814 | 556 | 557 |
| 815 | 584 | 585 |
| 816 | 582 | 583 |
| 817 | 526 | 527 |
| 818 | 554 | 555 |
| 819 | 552 | 553 |
| 820 | 540 | 541 |
| 821 | 568 | 569 |
| 822 | 566 | 567 |
| 823 | 556 | 557 |
| 824 | 584 | 585 |
| 825 | 582 | 583 |
| 826 | 519 | 520 |
| 827 | 547 | 548 |
| 828 | 545 | 546 |
| 829 | 533 | 534 |
| 830 | 561 | 562 |
| 831 | 559 | 560 |
| 832 | 549 | 550 |
| 833 | 577 | 578 |
| 834 | 575 | 576 |
| 835 | 534 | 535 |
| 836 | 562 | 563 |
| 837 | 560 | 561 |
| 838 | 548 | 549 |
| 839 | 576 | 577 |
| 840 | 574 | 575 |
| 841 | 564 | 565 |
| 842 | 592 | 593 |
| 843 | 590 | 591 |
| 844 | 569 | 570 |
| 845 | 597 | 598 |
| 846 | 595 | 596 |
| 847 | 583 | 584 |
| 848 | 611 | 612 |
| 849 | 609 | 610 |
| 850 | 599 | 600 |
| 851 | 627 | 628 |
| 852 | 625 | 626 |
| 853 | 603 | 604 |
| 854 | 631 | 632 |
| 855 | 629 | 630 |
| 856 | 617 | 618 |
| 857 | 645 | 646 |
| 858 | 643 | 644 |
| 859 | 633 | 634 |
| 860 | 661 | 662 |
| 861 | 659 | 660 |
| 862 | 534 | 535 |
| 863 | 562 | 563 |
| 864 | 560 | 561 |
| 865 | 548 | 549 |
| 866 | 576 | 577 |
| 867 | 574 | 575 |
| 868 | 564 | 565 |
| 869 | 592 | 593 |
| 870 | 590 | 591 |
| 871 | 534 | 535 |
| 872 | 562 | 563 |
| 873 | 560 | 561 |
| 874 | 548 | 549 |
| 875 | 576 | 577 |
| 876 | 574 | 575 |
| 877 | 564 | 565 |
| 878 | 592 | 593 |
| 879 | 590 | 591 |
| 880 | 484 | 485 |
| 881 | 512 | 513 |
| 882 | 510 | 511 |
| 883 | 498 | 499 |
| 884 | 526 | 527 |
| 885 | 524 | 525 |
| 886 | 514 | 515 |
| 887 | 542 | 543 |
| 888 | 540 | 541 |
| 889 | 484 | 485 |
| 890 | 512 | 513 |
| 891 | 510 | 511 |
| 892 | 498 | 499 |
| 893 | 526 | 527 |
| 894 | 524 | 525 |
| 895 | 514 | 515 |
| 896 | 542 | 543 |
| 897 | 540 | 541 |
| 898 | 534 | 535 |
| 899 | 562 | 563 |
| 900 | 560 | 561 |
| 901 | 548 | 549 |
| 902 | 576 | 577 |
| 903 | 574 | 575 |
| 904 | 564 | 565 |
| 905 | 592 | 593 |
| 906 | 590 | 591 |
| 907 | 534 | 535 |
| 908 | 562 | 563 |
| 909 | 560 | 561 |
| 910 | 548 | 549 |
| 911 | 576 | 577 |
| 912 | 574 | 575 |
| 913 | 564 | 565 |
| 914 | 592 | 593 |
| 915 | 590 | 591 |
| 916 | 519 | 520 |
| 917 | 547 | 548 |
| 918 | 545 | 546 |
| 919 | 533 | 534 |
| 920 | 561 | 562 |
| 921 | 559 | 560 |
| 922 | 549 | 550 |
| 923 | 577 | 578 |
| 924 | 575 | 576 |
| 925 | 519 | 520 |
| 926 | 547 | 548 |
| 927 | 545 | 546 |
| 928 | 533 | 534 |
| 929 | 561 | 562 |
| 930 | 559 | 560 |
| 931 | 549 | 550 |
| 932 | 577 | 578 |
| 933 | 575 | 576 |
| 934 | 537 | 538 |
| 935 | 565 | 566 |
| 936 | 563 | 564 |
| 937 | 551 | 552 |
| 938 | 579 | 580 |
| 939 | 577 | 578 |
| 940 | 567 | 568 |
| 941 | 595 | 596 |
| 942 | 593 | 594 |
| 943 | 573 | 574 |
| 944 | 601 | 602 |
| 945 | 599 | 600 |
| 946 | 587 | 588 |
| 947 | 615 | 616 |
| 948 | 613 | 614 |
| 949 | 603 | 604 |
| 950 | 631 | 632 |
| 951 | 629 | 630 |
| 952 | 501 | 502 |
| 953 | 529 | 530 |
| 954 | 527 | 528 |
| 955 | 515 | 516 |
| 956 | 543 | 544 |
| 957 | 541 | 542 |
| 958 | 531 | 532 |
| 959 | 559 | 560 |
| 960 | 557 | 558 |
| 961 | 501 | 502 |
| 962 | 529 | 530 |
| 963 | 527 | 528 |
| 964 | 515 | 516 |
| 965 | 543 | 544 |
| 966 | 541 | 542 |
| 967 | 531 | 532 |
| 968 | 559 | 560 |
| 969 | 557 | 558 |
| 970 | 501 | 502 |
| 971 | 529 | 530 |
| 972 | 527 | 528 |
| 973 | 515 | 516 |
| 974 | 543 | 544 |
| 975 | 541 | 542 |
| 976 | 531 | 532 |
| 977 | 559 | 560 |
| 978 | 557 | 558 |
| 979 | 552 | 553 |
| 980 | 580 | 581 |
| 981 | 578 | 579 |
| 982 | 566 | 567 |
| 983 | 594 | 595 |
| 984 | 592 | 593 |
| 985 | 582 | 583 |
| 986 | 610 | 611 |
| 987 | 608 | 609 |
| 988 | 566 | 567 |
| 989 | 594 | 595 |
| 990 | 592 | 593 |
| 991 | 580 | 581 |
| 992 | 608 | 609 |
| 993 | 606 | 607 |
| 994 | 596 | 597 |
| 995 | 624 | 625 |
| 996 | 622 | 623 |
| 997 | 523 | 524 |
| 998 | 551 | 552 |
| 999 | 549 | 550 |
| 1000 | 537 | 538 |
| 1001 | 565 | 566 |
| 1002 | 563 | 564 |
| 1003 | 553 | 554 |
| 1004 | 581 | 582 |
| 1005 | 579 | 580 |
| 1006 | 537 | 538 |
| 1007 | 565 | 566 |
| 1008 | 563 | 564 |
| 1009 | 551 | 552 |
| 1010 | 579 | 580 |
| 1011 | 577 | 578 |
| 1012 | 567 | 568 |
| 1013 | 595 | 596 |
| 1014 | 593 | 594 |
| 1015 | 523 | 524 |
| 1016 | 551 | 552 |
| 1017 | 549 | 550 |
| 1018 | 537 | 538 |
| 1019 | 565 | 566 |
| 1020 | 563 | 564 |
| 1021 | 553 | 554 |
| 1022 | 581 | 582 |
| 1023 | 579 | 580 |
| 1024 | 537 | 538 |
| 1025 | 565 | 566 |
| 1026 | 563 | 564 |
| 1027 | 551 | 552 |
| 1028 | 579 | 580 |
| 1029 | 577 | 578 |
| 1030 | 567 | 568 |
| 1031 | 595 | 596 |
| 1032 | 593 | 594 |
| 1033 | 561 | 562 |
| 1034 | 589 | 590 |
| 1035 | 587 | 588 |
| 1036 | 575 | 576 |
| 1037 | 603 | 604 |
| 1038 | 601 | 602 |
| 1039 | 591 | 592 |
| 1040 | 619 | 620 |
| 1041 | 617 | 618 |
| 1042 | 523 | 524 |
| 1043 | 551 | 552 |
| 1044 | 549 | 550 |
| 1045 | 537 | 538 |
| 1046 | 565 | 566 |
| 1047 | 563 | 564 |
| 1048 | 553 | 554 |
| 1049 | 581 | 582 |
| 1050 | 579 | 580 |
| 1051 | 537 | 538 |
| 1052 | 565 | 566 |
| 1053 | 563 | 564 |
| 1054 | 551 | 552 |
| 1055 | 579 | 580 |
| 1056 | 577 | 578 |
| 1057 | 567 | 568 |
| 1058 | 595 | 596 |
| 1059 | 593 | 594 |
| 1060 | 671 | 672 |
| 1061 | 699 | 700 |
| 1062 | 697 | 698 |
| 1063 | 685 | 686 |
| 1064 | 713 | 714 |
| 1065 | 711 | 712 |
| 1066 | 701 | 702 |
| 1067 | 729 | 730 |
| 1068 | 727 | 728 |
| 1069 | 561 | 562 |
| 1070 | 589 | 590 |
| 1071 | 587 | 588 |
| 1072 | 575 | 576 |
| 1073 | 603 | 604 |
| 1074 | 601 | 602 |
| 1075 | 591 | 592 |
| 1076 | 619 | 620 |
| 1077 | 617 | 618 |
| 1078 | 561 | 562 |
| 1079 | 589 | 590 |
| 1080 | 587 | 588 |
| 1081 | 575 | 576 |
| 1082 | 603 | 604 |
| 1083 | 601 | 602 |
| 1084 | 591 | 592 |
| 1085 | 619 | 620 |
| 1086 | 617 | 618 |
| 1087 | 524 | 525 |
| 1088 | 552 | 553 |
| 1089 | 550 | 551 |
| 1090 | 538 | 539 |
| 1091 | 566 | 567 |
| 1092 | 564 | 565 |
| 1093 | 554 | 555 |
| 1094 | 582 | 583 |
| 1095 | 580 | 581 |
| 1096 | 538 | 539 |
| 1097 | 566 | 567 |
| 1098 | 564 | 565 |
| 1099 | 552 | 553 |
| 1100 | 580 | 581 |
| 1101 | 578 | 579 |
| 1102 | 568 | 569 |
| 1103 | 596 | 597 |
| 1104 | 594 | 595 |
| 1105 | 538 | 539 |
| 1106 | 566 | 567 |
| 1107 | 564 | 565 |
| 1108 | 552 | 553 |
| 1109 | 580 | 581 |
| 1110 | 578 | 579 |
| 1111 | 568 | 569 |
| 1112 | 596 | 597 |
| 1113 | 594 | 595 |
| 1114 | 540 | 541 |
| 1115 | 568 | 569 |
| 1116 | 566 | 567 |
| 1117 | 554 | 555 |
| 1118 | 582 | 583 |
| 1119 | 580 | 581 |
| 1120 | 570 | 571 |
| 1121 | 598 | 599 |
| 1122 | 596 | 597 |
| 1123 | 523 | 524 |
| 1124 | 551 | 552 |
| 1125 | 549 | 550 |
| 1126 | 537 | 538 |
| 1127 | 565 | 566 |
| 1128 | 563 | 564 |
| 1129 | 553 | 554 |
| 1130 | 581 | 582 |
| 1131 | 579 | 580 |
| 1132 | 539 | 540 |
| 1133 | 567 | 568 |
| 1134 | 565 | 566 |
| 1135 | 553 | 554 |
| 1136 | 581 | 582 |
| 1137 | 579 | 580 |
| 1138 | 569 | 570 |
| 1139 | 597 | 598 |
| 1140 | 595 | 596 |
| 1141 | 539 | 540 |
| 1142 | 567 | 568 |
| 1143 | 565 | 566 |
| 1144 | 553 | 554 |
| 1145 | 581 | 582 |
| 1146 | 579 | 580 |
| 1147 | 569 | 570 |
| 1148 | 597 | 598 |
| 1149 | 595 | 596 |
| 1150 | 540 | 541 |
| 1151 | 568 | 569 |
| 1152 | 566 | 567 |
| 1153 | 554 | 555 |
| 1154 | 582 | 583 |
| 1155 | 580 | 581 |
| 1156 | 570 | 571 |
| 1157 | 598 | 599 |
| 1158 | 596 | 597 |
| 1159 | 524 | 525 |
| 1160 | 552 | 553 |
| 1161 | 550 | 551 |
| 1162 | 538 | 539 |
| 1163 | 566 | 567 |
| 1164 | 564 | 565 |
| 1165 | 554 | 555 |
| 1166 | 582 | 583 |
| 1167 | 580 | 581 |
| 1168 | 540 | 541 |
| 1169 | 568 | 569 |
| 1170 | 566 | 567 |
| 1171 | 554 | 555 |
| 1172 | 582 | 583 |
| 1173 | 580 | 581 |
| 1174 | 570 | 571 |
| 1175 | 598 | 599 |
| 1176 | 596 | 597 |
| 1177 | 554 | 555 |
| 1178 | 582 | 583 |
| 1179 | 580 | 581 |
| 1180 | 568 | 569 |
| 1181 | 596 | 597 |
| 1182 | 594 | 595 |
| 1183 | 584 | 585 |
| 1184 | 612 | 613 |
| 1185 | 610 | 611 |
| 1186 | 583 | 584 |
| 1187 | 611 | 612 |
| 1188 | 609 | 610 |
| 1189 | 597 | 598 |
| 1190 | 625 | 626 |
| 1191 | 623 | 624 |
| 1192 | 613 | 614 |
| 1193 | 641 | 642 |
| 1194 | 639 | 640 |
| 1195 | 558 | 559 |
| 1196 | 586 | 587 |
| 1197 | 584 | 585 |
| 1198 | 572 | 573 |
| 1199 | 600 | 601 |
| 1200 | 598 | 599 |
| 1201 | 555 | 556 |
| 1202 | 573 | 574 |
| 1203 | 585 | 586 |
| 1204 | 624 | 625 |
| 1205 | 479 | 480 |
| 1206 | 521 | 522 |
| 1207 | 583 | 584 |
| 1208 | 601 | 602 |
| 1209 | 613 | 614 |
| 1210 | 652 | 653 |
| 1211 | 507 | 508 |
| 1212 | 549 | 550 |
| 1213 | 581 | 582 |
| 1214 | 599 | 600 |
| 1215 | 611 | 612 |
| 1216 | 650 | 651 |
| 1217 | 505 | 506 |
| 1218 | 547 | 548 |
| 1219 | 579 | 580 |
| 1220 | 597 | 598 |
| 1221 | 609 | 610 |
| 1222 | 648 | 649 |
| 1223 | 503 | 504 |
| 1224 | 545 | 546 |
| 1225 | 583 | 584 |
| 1226 | 601 | 602 |
| 1227 | 613 | 614 |
| 1228 | 652 | 653 |
| 1229 | 507 | 508 |
| 1230 | 549 | 550 |
| 1231 | 580 | 581 |
| 1232 | 598 | 599 |
| 1233 | 610 | 611 |
| 1234 | 649 | 650 |
| 1235 | 504 | 505 |
| 1236 | 546 | 547 |
| 1237 | 569 | 570 |
| 1238 | 587 | 588 |
| 1239 | 599 | 600 |
| 1240 | 638 | 639 |
| 1241 | 493 | 494 |
| 1242 | 535 | 536 |
| 1243 | 597 | 598 |
| 1244 | 615 | 616 |
| 1245 | 627 | 628 |
| 1246 | 666 | 667 |
| 1247 | 521 | 522 |
| 1248 | 563 | 564 |
| 1249 | 595 | 596 |
| 1250 | 613 | 614 |
| 1251 | 625 | 626 |
| 1252 | 664 | 665 |
| 1253 | 519 | 520 |
| 1254 | 561 | 562 |
| 1255 | 593 | 594 |
| 1256 | 611 | 612 |
| 1257 | 623 | 624 |
| 1258 | 662 | 663 |
| 1259 | 517 | 518 |
| 1260 | 559 | 560 |
| 1261 | 597 | 598 |
| 1262 | 615 | 616 |
| 1263 | 627 | 628 |
| 1264 | 666 | 667 |
| 1265 | 521 | 522 |
| 1266 | 563 | 564 |
| 1267 | 594 | 595 |
| 1268 | 612 | 613 |
| 1269 | 624 | 625 |
| 1270 | 663 | 664 |
| 1271 | 518 | 519 |
| 1272 | 560 | 561 |
| 1273 | 585 | 586 |
| 1274 | 603 | 604 |
| 1275 | 615 | 616 |
| 1276 | 654 | 655 |
| 1277 | 509 | 510 |
| 1278 | 551 | 552 |
| 1279 | 613 | 614 |
| 1280 | 631 | 632 |
| 1281 | 643 | 644 |
| 1282 | 682 | 683 |
| 1283 | 537 | 538 |
| 1284 | 579 | 580 |
| 1285 | 611 | 612 |
| 1286 | 629 | 630 |
| 1287 | 641 | 642 |
| 1288 | 680 | 681 |
| 1289 | 535 | 536 |
| 1290 | 577 | 578 |
| 1291 | 609 | 610 |
| 1292 | 627 | 628 |
| 1293 | 639 | 640 |
| 1294 | 678 | 679 |
| 1295 | 533 | 534 |
| 1296 | 575 | 576 |
| 1297 | 613 | 614 |
| 1298 | 631 | 632 |
| 1299 | 643 | 644 |
| 1300 | 682 | 683 |
| 1301 | 537 | 538 |
| 1302 | 579 | 580 |
| 1303 | 610 | 611 |
| 1304 | 628 | 629 |
| 1305 | 640 | 641 |
| 1306 | 679 | 680 |
| 1307 | 534 | 535 |
| 1308 | 576 | 577 |
| 1309 | 601 | 602 |
| 1310 | 619 | 620 |
| 1311 | 631 | 632 |
| 1312 | 670 | 671 |
| 1313 | 525 | 526 |
| 1314 | 567 | 568 |
| 1315 | 629 | 630 |
| 1316 | 647 | 648 |
| 1317 | 659 | 660 |
| 1318 | 698 | 699 |
| 1319 | 553 | 554 |
| 1320 | 595 | 596 |
| 1321 | 627 | 628 |
| 1322 | 645 | 646 |
| 1323 | 657 | 658 |
| 1324 | 696 | 697 |
| 1325 | 551 | 552 |
| 1326 | 593 | 594 |
| 1327 | 625 | 626 |
| 1328 | 643 | 644 |
| 1329 | 655 | 656 |
| 1330 | 694 | 695 |
| 1331 | 549 | 550 |
| 1332 | 591 | 592 |
| 1333 | 629 | 630 |
| 1334 | 647 | 648 |
| 1335 | 659 | 660 |
| 1336 | 698 | 699 |
| 1337 | 553 | 554 |
| 1338 | 595 | 596 |
| 1339 | 626 | 627 |
| 1340 | 644 | 645 |
| 1341 | 656 | 657 |
| 1342 | 695 | 696 |
| 1343 | 550 | 551 |
| 1344 | 592 | 593 |
| 1345 | 584 | 585 |
| 1346 | 602 | 603 |
| 1347 | 614 | 615 |
| 1348 | 653 | 654 |
| 1349 | 508 | 509 |
| 1350 | 550 | 551 |
| 1351 | 612 | 613 |
| 1352 | 630 | 631 |
| 1353 | 642 | 643 |
| 1354 | 681 | 682 |
| 1355 | 536 | 537 |
| 1356 | 578 | 579 |
| 1357 | 610 | 611 |
| 1358 | 628 | 629 |
| 1359 | 640 | 641 |
| 1360 | 679 | 680 |
| 1361 | 534 | 535 |
| 1362 | 576 | 577 |
| 1363 | 608 | 609 |
| 1364 | 626 | 627 |
| 1365 | 638 | 639 |
| 1366 | 677 | 678 |
| 1367 | 532 | 533 |
| 1368 | 574 | 575 |
| 1369 | 612 | 613 |
| 1370 | 630 | 631 |
| 1371 | 642 | 643 |
| 1372 | 681 | 682 |
| 1373 | 536 | 537 |
| 1374 | 578 | 579 |
| 1375 | 609 | 610 |
| 1376 | 627 | 628 |
| 1377 | 639 | 640 |
| 1378 | 678 | 679 |
| 1379 | 533 | 534 |
| 1380 | 575 | 576 |
| 1381 | 612 | 613 |
| 1382 | 630 | 631 |
| 1383 | 642 | 643 |
| 1384 | 681 | 682 |
| 1385 | 536 | 537 |
| 1386 | 578 | 579 |
| 1387 | 640 | 641 |
| 1388 | 658 | 659 |
| 1389 | 670 | 671 |
| 1390 | 709 | 710 |
| 1391 | 564 | 565 |
| 1392 | 606 | 607 |
| 1393 | 638 | 639 |
| 1394 | 656 | 657 |
| 1395 | 668 | 669 |
| 1396 | 707 | 708 |
| 1397 | 562 | 563 |
| 1398 | 604 | 605 |
| 1399 | 636 | 637 |
| 1400 | 654 | 655 |
| 1401 | 666 | 667 |
| 1402 | 705 | 706 |
| 1403 | 560 | 561 |
| 1404 | 602 | 603 |
| 1405 | 640 | 641 |
| 1406 | 658 | 659 |
| 1407 | 670 | 671 |
| 1408 | 709 | 710 |
| 1409 | 564 | 565 |
| 1410 | 606 | 607 |
| 1411 | 637 | 638 |
| 1412 | 655 | 656 |
| 1413 | 667 | 668 |
| 1414 | 706 | 707 |
| 1415 | 561 | 562 |
| 1416 | 603 | 604 |
| 1417 | 688 | 689 |
| 1418 | 706 | 707 |
| 1419 | 718 | 719 |
| 1420 | 757 | 758 |
| 1421 | 612 | 613 |
| 1422 | 654 | 655 |
| 1423 | 716 | 717 |
| 1424 | 734 | 735 |
| 1425 | 746 | 747 |
| 1426 | 785 | 786 |
| 1427 | 640 | 641 |
| 1428 | 682 | 683 |
| 1429 | 714 | 715 |
| 1430 | 732 | 733 |
| 1431 | 744 | 745 |
| 1432 | 783 | 784 |
| 1433 | 638 | 639 |
| 1434 | 680 | 681 |
| 1435 | 712 | 713 |
| 1436 | 730 | 731 |
| 1437 | 742 | 743 |
| 1438 | 781 | 782 |
| 1439 | 636 | 637 |
| 1440 | 678 | 679 |
| 1441 | 716 | 717 |
| 1442 | 734 | 735 |
| 1443 | 746 | 747 |
| 1444 | 785 | 786 |
| 1445 | 640 | 641 |
| 1446 | 682 | 683 |
| 1447 | 713 | 714 |
| 1448 | 731 | 732 |
| 1449 | 743 | 744 |
| 1450 | 782 | 783 |
| 1451 | 637 | 638 |
| 1452 | 679 | 680 |
| 1453 | 718 | 719 |
| 1454 | 736 | 737 |
| 1455 | 748 | 749 |
| 1456 | 787 | 788 |
| 1457 | 642 | 643 |
| 1458 | 684 | 685 |
| 1459 | 746 | 747 |
| 1460 | 764 | 765 |
| 1461 | 776 | 777 |
| 1462 | 815 | 816 |
| 1463 | 670 | 671 |
| 1464 | 712 | 713 |
| 1465 | 744 | 745 |
| 1466 | 762 | 763 |
| 1467 | 774 | 775 |
| 1468 | 813 | 814 |
| 1469 | 668 | 669 |
| 1470 | 710 | 711 |
| 1471 | 742 | 743 |
| 1472 | 760 | 761 |
| 1473 | 772 | 773 |
| 1474 | 811 | 812 |
| 1475 | 666 | 667 |
| 1476 | 708 | 709 |
| 1477 | 746 | 747 |
| 1478 | 764 | 765 |
| 1479 | 776 | 777 |
| 1480 | 815 | 816 |
| 1481 | 670 | 671 |
| 1482 | 712 | 713 |
| 1483 | 743 | 744 |
| 1484 | 761 | 762 |
| 1485 | 773 | 774 |
| 1486 | 812 | 813 |
| 1487 | 667 | 668 |
| 1488 | 709 | 710 |
| 1489 | 612 | 613 |
| 1490 | 630 | 631 |
| 1491 | 642 | 643 |
| 1492 | 681 | 682 |
| 1493 | 536 | 537 |
| 1494 | 578 | 579 |
| 1495 | 640 | 641 |
| 1496 | 658 | 659 |
| 1497 | 670 | 671 |
| 1498 | 709 | 710 |
| 1499 | 564 | 565 |
| 1500 | 606 | 607 |
| 1501 | 638 | 639 |
| 1502 | 656 | 657 |
| 1503 | 668 | 669 |
| 1504 | 707 | 708 |
| 1505 | 562 | 563 |
| 1506 | 604 | 605 |
| 1507 | 636 | 637 |
| 1508 | 654 | 655 |
| 1509 | 666 | 667 |
| 1510 | 705 | 706 |
| 1511 | 560 | 561 |
| 1512 | 602 | 603 |
| 1513 | 640 | 641 |
| 1514 | 658 | 659 |
| 1515 | 670 | 671 |
| 1516 | 709 | 710 |
| 1517 | 564 | 565 |
| 1518 | 606 | 607 |
| 1519 | 637 | 638 |
| 1520 | 655 | 656 |
| 1521 | 667 | 668 |
| 1522 | 706 | 707 |
| 1523 | 561 | 562 |
| 1524 | 603 | 604 |
| 1525 | 614 | 615 |
| 1526 | 632 | 633 |
| 1527 | 644 | 645 |
| 1528 | 683 | 684 |
| 1529 | 538 | 539 |
| 1530 | 580 | 581 |
| 1531 | 642 | 643 |
| 1532 | 660 | 661 |
| 1533 | 672 | 673 |
| 1534 | 711 | 712 |
| 1535 | 566 | 567 |
| 1536 | 608 | 609 |
| 1537 | 640 | 641 |
| 1538 | 658 | 659 |
| 1539 | 670 | 671 |
| 1540 | 709 | 710 |
| 1541 | 564 | 565 |
| 1542 | 606 | 607 |
| 1543 | 638 | 639 |
| 1544 | 656 | 657 |
| 1545 | 668 | 669 |
| 1546 | 707 | 708 |
| 1547 | 562 | 563 |
| 1548 | 604 | 605 |
| 1549 | 642 | 643 |
| 1550 | 660 | 661 |
| 1551 | 672 | 673 |
| 1552 | 711 | 712 |
| 1553 | 566 | 567 |
| 1554 | 608 | 609 |
| 1555 | 639 | 640 |
| 1556 | 657 | 658 |
| 1557 | 669 | 670 |
| 1558 | 708 | 709 |
| 1559 | 563 | 564 |
| 1560 | 605 | 606 |
| 1561 | 599 | 600 |
| 1562 | 617 | 618 |
| 1563 | 629 | 630 |
| 1564 | 668 | 669 |
| 1565 | 523 | 524 |
| 1566 | 565 | 566 |
| 1567 | 627 | 628 |
| 1568 | 645 | 646 |
| 1569 | 657 | 658 |
| 1570 | 696 | 697 |
| 1571 | 551 | 552 |
| 1572 | 593 | 594 |
| 1573 | 625 | 626 |
| 1574 | 643 | 644 |
| 1575 | 655 | 656 |
| 1576 | 694 | 695 |
| 1577 | 549 | 550 |
| 1578 | 591 | 592 |
| 1579 | 623 | 624 |
| 1580 | 641 | 642 |
| 1581 | 653 | 654 |
| 1582 | 692 | 693 |
| 1583 | 547 | 548 |
| 1584 | 589 | 590 |
| 1585 | 627 | 628 |
| 1586 | 645 | 646 |
| 1587 | 657 | 658 |
| 1588 | 696 | 697 |
| 1589 | 551 | 552 |
| 1590 | 593 | 594 |
| 1591 | 624 | 625 |
| 1592 | 642 | 643 |
| 1593 | 654 | 655 |
| 1594 | 693 | 694 |
| 1595 | 548 | 549 |
| 1596 | 590 | 591 |
| 1597 | 657 | 658 |
| 1598 | 675 | 676 |
| 1599 | 687 | 688 |
| 1600 | 726 | 727 |
| 1601 | 581 | 582 |
| 1602 | 623 | 624 |
| 1603 | 685 | 686 |
| 1604 | 703 | 704 |
| 1605 | 715 | 716 |
| 1606 | 754 | 755 |
| 1607 | 609 | 610 |
| 1608 | 651 | 652 |
| 1609 | 683 | 684 |
| 1610 | 701 | 702 |
| 1611 | 713 | 714 |
| 1612 | 752 | 753 |
| 1613 | 607 | 608 |
| 1614 | 649 | 650 |
| 1615 | 681 | 682 |
| 1616 | 699 | 700 |
| 1617 | 711 | 712 |
| 1618 | 750 | 751 |
| 1619 | 605 | 606 |
| 1620 | 647 | 648 |
| 1621 | 685 | 686 |
| 1622 | 703 | 704 |
| 1623 | 715 | 716 |
| 1624 | 754 | 755 |
| 1625 | 609 | 610 |
| 1626 | 651 | 652 |
| 1627 | 682 | 683 |
| 1628 | 700 | 701 |
| 1629 | 712 | 713 |
| 1630 | 751 | 752 |
| 1631 | 606 | 607 |
| 1632 | 648 | 649 |
| 1633 | 738 | 739 |
| 1634 | 756 | 757 |
| 1635 | 768 | 769 |
| 1636 | 807 | 808 |
| 1637 | 662 | 663 |
| 1638 | 704 | 705 |
| 1639 | 766 | 767 |
| 1640 | 784 | 785 |
| 1641 | 796 | 797 |
| 1642 | 835 | 836 |
| 1643 | 690 | 691 |
| 1644 | 732 | 733 |
| 1645 | 764 | 765 |
| 1646 | 782 | 783 |
| 1647 | 794 | 795 |
| 1648 | 833 | 834 |
| 1649 | 688 | 689 |
| 1650 | 730 | 731 |
| 1651 | 762 | 763 |
| 1652 | 780 | 781 |
| 1653 | 792 | 793 |
| 1654 | 831 | 832 |
| 1655 | 686 | 687 |
| 1656 | 728 | 729 |
| 1657 | 766 | 767 |
| 1658 | 784 | 785 |
| 1659 | 796 | 797 |
| 1660 | 835 | 836 |
| 1661 | 690 | 691 |
| 1662 | 732 | 733 |
| 1663 | 763 | 764 |
| 1664 | 781 | 782 |
| 1665 | 793 | 794 |
| 1666 | 832 | 833 |
| 1667 | 687 | 688 |
| 1668 | 729 | 730 |
| 1669 | 587 | 588 |
| 1670 | 605 | 606 |
| 1671 | 617 | 618 |
| 1672 | 656 | 657 |
| 1673 | 511 | 512 |
| 1674 | 553 | 554 |
| 1675 | 615 | 616 |
| 1676 | 633 | 634 |
| 1677 | 645 | 646 |
| 1678 | 684 | 685 |
| 1679 | 539 | 540 |
| 1680 | 581 | 582 |
| 1681 | 613 | 614 |
| 1682 | 631 | 632 |
| 1683 | 643 | 644 |
| 1684 | 682 | 683 |
| 1685 | 537 | 538 |
| 1686 | 579 | 580 |
| 1687 | 611 | 612 |
| 1688 | 629 | 630 |
| 1689 | 641 | 642 |
| 1690 | 680 | 681 |
| 1691 | 535 | 536 |
| 1692 | 577 | 578 |
| 1693 | 615 | 616 |
| 1694 | 633 | 634 |
| 1695 | 645 | 646 |
| 1696 | 684 | 685 |
| 1697 | 539 | 540 |
| 1698 | 581 | 582 |
| 1699 | 612 | 613 |
| 1700 | 630 | 631 |
| 1701 | 642 | 643 |
| 1702 | 681 | 682 |
| 1703 | 536 | 537 |
| 1704 | 578 | 579 |
| 1705 | 600 | 601 |
| 1706 | 618 | 619 |
| 1707 | 630 | 631 |
| 1708 | 669 | 670 |
| 1709 | 524 | 525 |
| 1710 | 566 | 567 |
| 1711 | 628 | 629 |
| 1712 | 646 | 647 |
| 1713 | 658 | 659 |
| 1714 | 697 | 698 |
| 1715 | 552 | 553 |
| 1716 | 594 | 595 |
| 1717 | 626 | 627 |
| 1718 | 644 | 645 |
| 1719 | 656 | 657 |
| 1720 | 695 | 696 |
| 1721 | 550 | 551 |
| 1722 | 592 | 593 |
| 1723 | 624 | 625 |
| 1724 | 642 | 643 |
| 1725 | 654 | 655 |
| 1726 | 693 | 694 |
| 1727 | 548 | 549 |
| 1728 | 590 | 591 |
| 1729 | 628 | 629 |
| 1730 | 646 | 647 |
| 1731 | 658 | 659 |
| 1732 | 697 | 698 |
| 1733 | 552 | 553 |
| 1734 | 594 | 595 |
| 1735 | 625 | 626 |
| 1736 | 643 | 644 |
| 1737 | 655 | 656 |
| 1738 | 694 | 695 |
| 1739 | 549 | 550 |
| 1740 | 591 | 592 |
| 1741 | 656 | 657 |
| 1742 | 674 | 675 |
| 1743 | 686 | 687 |
| 1744 | 725 | 726 |
| 1745 | 580 | 581 |
| 1746 | 622 | 623 |
| 1747 | 684 | 685 |
| 1748 | 702 | 703 |
| 1749 | 714 | 715 |
| 1750 | 753 | 754 |
| 1751 | 608 | 609 |
| 1752 | 650 | 651 |
| 1753 | 682 | 683 |
| 1754 | 700 | 701 |
| 1755 | 712 | 713 |
| 1756 | 751 | 752 |
| 1757 | 606 | 607 |
| 1758 | 648 | 649 |
| 1759 | 680 | 681 |
| 1760 | 698 | 699 |
| 1761 | 710 | 711 |
| 1762 | 749 | 750 |
| 1763 | 604 | 605 |
| 1764 | 646 | 647 |
| 1765 | 684 | 685 |
| 1766 | 702 | 703 |
| 1767 | 714 | 715 |
| 1768 | 753 | 754 |
| 1769 | 608 | 609 |
| 1770 | 650 | 651 |
| 1771 | 681 | 682 |
| 1772 | 699 | 700 |
| 1773 | 711 | 712 |
| 1774 | 750 | 751 |
| 1775 | 605 | 606 |
| 1776 | 647 | 648 |
| 1777 | 699 | 700 |
| 1778 | 717 | 718 |
| 1779 | 729 | 730 |
| 1780 | 768 | 769 |
| 1781 | 623 | 624 |
| 1782 | 665 | 666 |
| 1783 | 727 | 728 |
| 1784 | 745 | 746 |
| 1785 | 757 | 758 |
| 1786 | 796 | 797 |
| 1787 | 651 | 652 |
| 1788 | 693 | 694 |
| 1789 | 725 | 726 |
| 1790 | 743 | 744 |
| 1791 | 755 | 756 |
| 1792 | 794 | 795 |
| 1793 | 649 | 650 |
| 1794 | 691 | 692 |
| 1795 | 723 | 724 |
| 1796 | 741 | 742 |
| 1797 | 753 | 754 |
| 1798 | 792 | 793 |
| 1799 | 647 | 648 |
| 1800 | 689 | 690 |
| 1801 | 727 | 728 |
| 1802 | 745 | 746 |
| 1803 | 757 | 758 |
| 1804 | 796 | 797 |
| 1805 | 651 | 652 |
| 1806 | 693 | 694 |
| 1807 | 724 | 725 |
| 1808 | 742 | 743 |
| 1809 | 754 | 755 |
| 1810 | 793 | 794 |
| 1811 | 648 | 649 |
| 1812 | 690 | 691 |
| 1813 | 629 | 630 |
| 1814 | 647 | 648 |
| 1815 | 659 | 660 |
| 1816 | 698 | 699 |
| 1817 | 553 | 554 |
| 1818 | 595 | 596 |
| 1819 | 657 | 658 |
| 1820 | 675 | 676 |
| 1821 | 687 | 688 |
| 1822 | 726 | 727 |
| 1823 | 581 | 582 |
| 1824 | 623 | 624 |
| 1825 | 655 | 656 |
| 1826 | 673 | 674 |
| 1827 | 685 | 686 |
| 1828 | 724 | 725 |
| 1829 | 579 | 580 |
| 1830 | 621 | 622 |
| 1831 | 653 | 654 |
| 1832 | 671 | 672 |
| 1833 | 683 | 684 |
| 1834 | 722 | 723 |
| 1835 | 577 | 578 |
| 1836 | 619 | 620 |
| 1837 | 657 | 658 |
| 1838 | 675 | 676 |
| 1839 | 687 | 688 |
| 1840 | 726 | 727 |
| 1841 | 581 | 582 |
| 1842 | 623 | 624 |
| 1843 | 654 | 655 |
| 1844 | 672 | 673 |
| 1845 | 684 | 685 |
| 1846 | 723 | 724 |
| 1847 | 578 | 579 |
| 1848 | 620 | 621 |
| 1849 | 662 | 663 |
| 1850 | 680 | 681 |
| 1851 | 692 | 693 |
| 1852 | 731 | 732 |
| 1853 | 586 | 587 |
| 1854 | 628 | 629 |
| 1855 | 690 | 691 |
| 1856 | 708 | 709 |
| 1857 | 720 | 721 |
| 1858 | 759 | 760 |
| 1859 | 614 | 615 |
| 1860 | 656 | 657 |
| 1861 | 688 | 689 |
| 1862 | 706 | 707 |
| 1863 | 718 | 719 |
| 1864 | 757 | 758 |
| 1865 | 612 | 613 |
| 1866 | 654 | 655 |
| 1867 | 686 | 687 |
| 1868 | 704 | 705 |
| 1869 | 716 | 717 |
| 1870 | 755 | 756 |
| 1871 | 610 | 611 |
| 1872 | 652 | 653 |
| 1873 | 690 | 691 |
| 1874 | 708 | 709 |
| 1875 | 720 | 721 |
| 1876 | 759 | 760 |
| 1877 | 614 | 615 |
| 1878 | 656 | 657 |
| 1879 | 687 | 688 |
| 1880 | 705 | 706 |
| 1881 | 717 | 718 |
| 1882 | 756 | 757 |
| 1883 | 611 | 612 |
| 1884 | 653 | 654 |
| 1885 | 609 | 610 |
| 1886 | 627 | 628 |
| 1887 | 639 | 640 |
| 1888 | 678 | 679 |
| 1889 | 533 | 534 |
| 1890 | 575 | 576 |
| 1891 | 637 | 638 |
| 1892 | 655 | 656 |
| 1893 | 667 | 668 |
| 1894 | 706 | 707 |
| 1895 | 561 | 562 |
| 1896 | 603 | 604 |
| 1897 | 635 | 636 |
| 1898 | 653 | 654 |
| 1899 | 665 | 666 |
| 1900 | 704 | 705 |
| 1901 | 559 | 560 |
| 1902 | 601 | 602 |
| 1903 | 633 | 634 |
| 1904 | 651 | 652 |
| 1905 | 663 | 664 |
| 1906 | 702 | 703 |
| 1907 | 557 | 558 |
| 1908 | 599 | 600 |
| 1909 | 637 | 638 |
| 1910 | 655 | 656 |
| 1911 | 667 | 668 |
| 1912 | 706 | 707 |
| 1913 | 561 | 562 |
| 1914 | 603 | 604 |
| 1915 | 634 | 635 |
| 1916 | 652 | 653 |
| 1917 | 664 | 665 |
| 1918 | 703 | 704 |
| 1919 | 558 | 559 |
| 1920 | 600 | 601 |
| 1921 | 609 | 610 |
| 1922 | 627 | 628 |
| 1923 | 639 | 640 |
| 1924 | 678 | 679 |
| 1925 | 533 | 534 |
| 1926 | 575 | 576 |
| 1927 | 637 | 638 |
| 1928 | 655 | 656 |
| 1929 | 667 | 668 |
| 1930 | 706 | 707 |
| 1931 | 561 | 562 |
| 1932 | 603 | 604 |
| 1933 | 635 | 636 |
| 1934 | 653 | 654 |
| 1935 | 665 | 666 |
| 1936 | 704 | 705 |
| 1937 | 559 | 560 |
| 1938 | 601 | 602 |
| 1939 | 633 | 634 |
| 1940 | 651 | 652 |
| 1941 | 663 | 664 |
| 1942 | 702 | 703 |
| 1943 | 557 | 558 |
| 1944 | 599 | 600 |
| 1945 | 637 | 638 |
| 1946 | 655 | 656 |
| 1947 | 667 | 668 |
| 1948 | 706 | 707 |
| 1949 | 561 | 562 |
| 1950 | 603 | 604 |
| 1951 | 634 | 635 |
| 1952 | 652 | 653 |
| 1953 | 664 | 665 |
| 1954 | 703 | 704 |
| 1955 | 558 | 559 |
| 1956 | 600 | 601 |
| 1957 | 626 | 627 |
| 1958 | 644 | 645 |
| 1959 | 656 | 657 |
| 1960 | 695 | 696 |
| 1961 | 550 | 551 |
| 1962 | 592 | 593 |
| 1963 | 654 | 655 |
| 1964 | 672 | 673 |
| 1965 | 684 | 685 |
| 1966 | 723 | 724 |
| 1967 | 578 | 579 |
| 1968 | 620 | 621 |
| 1969 | 652 | 653 |
| 1970 | 670 | 671 |
| 1971 | 682 | 683 |
| 1972 | 721 | 722 |
| 1973 | 576 | 577 |
| 1974 | 618 | 619 |
| 1975 | 650 | 651 |
| 1976 | 668 | 669 |
| 1977 | 680 | 681 |
| 1978 | 719 | 720 |
| 1979 | 574 | 575 |
| 1980 | 616 | 617 |
| 1981 | 654 | 655 |
| 1982 | 672 | 673 |
| 1983 | 684 | 685 |
| 1984 | 723 | 724 |
| 1985 | 578 | 579 |
| 1986 | 620 | 621 |
| 1987 | 651 | 652 |
| 1988 | 669 | 670 |
| 1989 | 681 | 682 |
| 1990 | 720 | 721 |
| 1991 | 575 | 576 |
| 1992 | 617 | 618 |
| 1993 | 626 | 627 |
| 1994 | 644 | 645 |
| 1995 | 656 | 657 |
| 1996 | 695 | 696 |
| 1997 | 550 | 551 |
| 1998 | 592 | 593 |
| 1999 | 654 | 655 |
| 2000 | 672 | 673 |
| 2001 | 684 | 685 |
| 2002 | 723 | 724 |
| 2003 | 578 | 579 |
| 2004 | 620 | 621 |
| 2005 | 652 | 653 |
| 2006 | 670 | 671 |
| 2007 | 682 | 683 |
| 2008 | 721 | 722 |
| 2009 | 576 | 577 |
| 2010 | 618 | 619 |
| 2011 | 650 | 651 |
| 2012 | 668 | 669 |
| 2013 | 680 | 681 |
| 2014 | 719 | 720 |
| 2015 | 574 | 575 |
| 2016 | 616 | 617 |
| 2017 | 654 | 655 |
| 2018 | 672 | 673 |
| 2019 | 684 | 685 |
| 2020 | 723 | 724 |
| 2021 | 578 | 579 |
| 2022 | 620 | 621 |
| 2023 | 651 | 652 |
| 2024 | 669 | 670 |
| 2025 | 681 | 682 |
| 2026 | 720 | 721 |
| 2027 | 575 | 576 |
| 2028 | 617 | 618 |
| 2029 | 586 | 587 |
| 2030 | 604 | 605 |
| 2031 | 616 | 617 |
| 2032 | 655 | 656 |
| 2033 | 510 | 511 |
| 2034 | 552 | 553 |
| 2035 | 614 | 615 |
| 2036 | 632 | 633 |
| 2037 | 644 | 645 |
| 2038 | 683 | 684 |
| 2039 | 538 | 539 |
| 2040 | 580 | 581 |
| 2041 | 612 | 613 |
| 2042 | 630 | 631 |
| 2043 | 642 | 643 |
| 2044 | 681 | 682 |
| 2045 | 536 | 537 |
| 2046 | 578 | 579 |
| 2047 | 610 | 611 |
| 2048 | 628 | 629 |
| 2049 | 640 | 641 |
| 2050 | 679 | 680 |
| 2051 | 534 | 535 |
| 2052 | 576 | 577 |
| 2053 | 614 | 615 |
| 2054 | 632 | 633 |
| 2055 | 644 | 645 |
| 2056 | 683 | 684 |
| 2057 | 538 | 539 |
| 2058 | 580 | 581 |
| 2059 | 611 | 612 |
| 2060 | 629 | 630 |
| 2061 | 641 | 642 |
| 2062 | 680 | 681 |
| 2063 | 535 | 536 |
| 2064 | 577 | 578 |
| 2065 | 640 | 641 |
| 2066 | 658 | 659 |
| 2067 | 670 | 671 |
| 2068 | 709 | 710 |
| 2069 | 564 | 565 |
| 2070 | 606 | 607 |
| 2071 | 668 | 669 |
| 2072 | 686 | 687 |
| 2073 | 698 | 699 |
| 2074 | 737 | 738 |
| 2075 | 592 | 593 |
| 2076 | 634 | 635 |
| 2077 | 666 | 667 |
| 2078 | 684 | 685 |
| 2079 | 696 | 697 |
| 2080 | 735 | 736 |
| 2081 | 590 | 591 |
| 2082 | 632 | 633 |
| 2083 | 664 | 665 |
| 2084 | 682 | 683 |
| 2085 | 694 | 695 |
| 2086 | 733 | 734 |
| 2087 | 588 | 589 |
| 2088 | 630 | 631 |
| 2089 | 668 | 669 |
| 2090 | 686 | 687 |
| 2091 | 698 | 699 |
| 2092 | 737 | 738 |
| 2093 | 592 | 593 |
| 2094 | 634 | 635 |
| 2095 | 665 | 666 |
| 2096 | 683 | 684 |
| 2097 | 695 | 696 |
| 2098 | 734 | 735 |
| 2099 | 589 | 590 |
| 2100 | 631 | 632 |
| 2101 | 637 | 638 |
| 2102 | 655 | 656 |
| 2103 | 667 | 668 |
| 2104 | 706 | 707 |
| 2105 | 561 | 562 |
| 2106 | 603 | 604 |
| 2107 | 665 | 666 |
| 2108 | 683 | 684 |
| 2109 | 695 | 696 |
| 2110 | 734 | 735 |
| 2111 | 589 | 590 |
| 2112 | 631 | 632 |
| 2113 | 663 | 664 |
| 2114 | 681 | 682 |
| 2115 | 693 | 694 |
| 2116 | 732 | 733 |
| 2117 | 587 | 588 |
| 2118 | 629 | 630 |
| 2119 | 661 | 662 |
| 2120 | 679 | 680 |
| 2121 | 691 | 692 |
| 2122 | 730 | 731 |
| 2123 | 585 | 586 |
| 2124 | 627 | 628 |
| 2125 | 665 | 666 |
| 2126 | 683 | 684 |
| 2127 | 695 | 696 |
| 2128 | 734 | 735 |
| 2129 | 589 | 590 |
| 2130 | 631 | 632 |
| 2131 | 662 | 663 |
| 2132 | 680 | 681 |
| 2133 | 692 | 693 |
| 2134 | 731 | 732 |
| 2135 | 586 | 587 |
| 2136 | 628 | 629 |
| 2137 | 659 | 660 |
| 2138 | 677 | 678 |
| 2139 | 689 | 690 |
| 2140 | 728 | 729 |
| 2141 | 583 | 584 |
| 2142 | 625 | 626 |
| 2143 | 687 | 688 |
| 2144 | 705 | 706 |
| 2145 | 717 | 718 |
| 2146 | 756 | 757 |
| 2147 | 611 | 612 |
| 2148 | 653 | 654 |
| 2149 | 685 | 686 |
| 2150 | 703 | 704 |
| 2151 | 715 | 716 |
| 2152 | 754 | 755 |
| 2153 | 609 | 610 |
| 2154 | 651 | 652 |
| 2155 | 683 | 684 |
| 2156 | 701 | 702 |
| 2157 | 713 | 714 |
| 2158 | 752 | 753 |
| 2159 | 607 | 608 |
| 2160 | 649 | 650 |
| 2161 | 687 | 688 |
| 2162 | 705 | 706 |
| 2163 | 717 | 718 |
| 2164 | 756 | 757 |
| 2165 | 611 | 612 |
| 2166 | 653 | 654 |
| 2167 | 684 | 685 |
| 2168 | 702 | 703 |
| 2169 | 714 | 715 |
| 2170 | 753 | 754 |
| 2171 | 608 | 609 |
| 2172 | 650 | 651 |
| 2173 | 559 | 560 |
| 2174 | 577 | 578 |
| 2175 | 589 | 590 |
| 2176 | 628 | 629 |
| 2177 | 483 | 484 |
| 2178 | 525 | 526 |
| 2179 | 587 | 588 |
| 2180 | 605 | 606 |
| 2181 | 617 | 618 |
| 2182 | 656 | 657 |
| 2183 | 511 | 512 |
| 2184 | 553 | 554 |
| 2185 | 585 | 586 |
| 2186 | 603 | 604 |
| 2187 | 615 | 616 |
| 2188 | 654 | 655 |
| 2189 | 509 | 510 |
| 2190 | 551 | 552 |
| 2191 | 583 | 584 |
| 2192 | 601 | 602 |
| 2193 | 613 | 614 |
| 2194 | 652 | 653 |
| 2195 | 507 | 508 |
| 2196 | 549 | 550 |
| 2197 | 587 | 588 |
| 2198 | 605 | 606 |
| 2199 | 617 | 618 |
| 2200 | 656 | 657 |
| 2203 | 661 | 662 |
| 2204 | 673 | 674 |
| 2205 | 671 | 672 |
| 2206 | 669 | 670 |
| 2207 | 687 | 688 |
| 2208 | 683 | 684 |
| 2209 | 695 | 696 |
| 2210 | 693 | 592 |
| 2211 | 691 | 692 |
| 2212 | 709 | 710 |
| 2213 | 559 | 560 |
| 2214 | 701 | 702 |
| 2215 | 713 | 714 |
| 2216 | 711 | 712 |
| 2217 | 709 | 710 |

### Example 2

### Effect of ICG-001 on Pulmonary Fibrosis

Murine models of bleomycin induced fibrosis have been developed in order to study protic disease progression. Bleomycin induced murine fibrosis has been shown to lead to ferrant alveolar epithelial repair, with increased metaplastic alveolar cells that apparently do protic properly differentiate to a type I phenotype (Adamson and Bowden, Am J Pathol. 96:531-4, 1979). Utilizing this model, it is demonstrated in this Example that the Wnt/β-catenin pathway plays a critical role in the development of pulmonary fibrosis and validates that the exhibition of this pathway with ICG-001 represents a therapy for the treatment of pulmonary protic disease.

Using this murine model of pulmonary fibrosis in transgenic Bat-Gal mice, ICG-001 5 mg/Kg/day, administered via minipump) blocked >95% of bleomycin-induced TCF/β-catenin transcription. Furthermore, ICG-001 at this dose not only halted but reversed disease progression, as judged by reduced mortality, histopathology and endogenous gene expression.

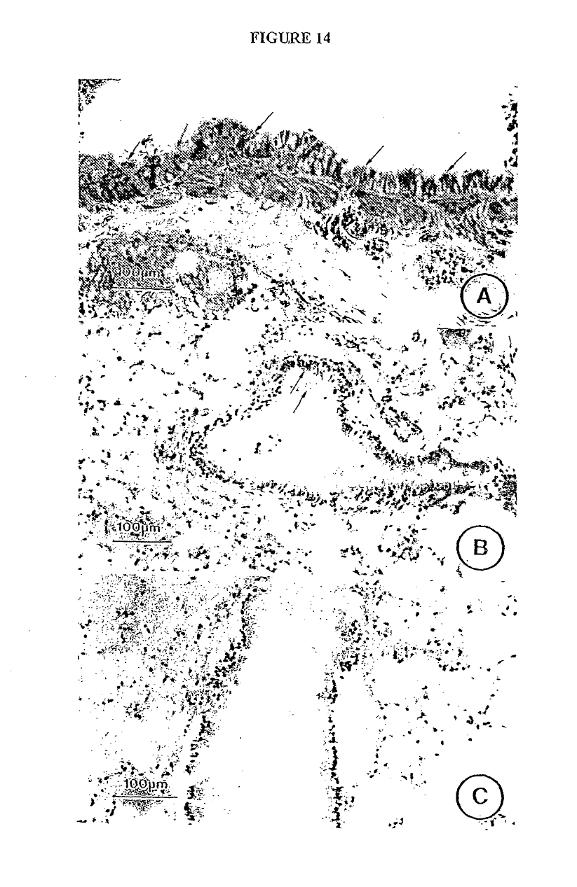

FIG. 14 shows lung sections taken from Bat-Gal transgenic mice. These mice have a Beta-Galactosidase transgene driven by a TCF/Catenin driven promoter (i.e. a read out for activated Wnt/catenin signaling). The mice were given intratracheal saline or bleomycin and either treated with ICG-001 (5 mgs/Kg/day subcutaneously) or saline as vehicle control. The mice were sacrificed and the lungs sectioned and stained with X-Gal (blue color) A) intratracheal bleo+saline. B) intracheal bleo+ICG-001 C) saline+saline.

The dose was selected because ICG-001 reduces TCF/β-Catenin driven β-Galactosidase expression >95% at 5 mgs/Kg/day.

FIG. 15 shows lung sections taken from C57/B16 mice treated with intratracheal bleomycin (lower left) or saline (upper left) for 5 days and stained with trichrome (red color) to stain collagen. There is an absence of airway epithelium in lower left compared to upper left (see arrow heads) and extensive collagen deposition (lower left). On the sixth day, either saline (upper right) or ICG-001(5 mgs/Kg/day) was administered for 10 days after which the mice were sacrificed and sectioned. Of interest is the upper right (saline treatment) showing lack of normal airway epithelialization, extensive collagen deposition and intra-airway hypercellularity (fibroblasts and inflammatory influx). After treatment with ICG-001, the airway looks essentially normal (compare to untreated (saline) control) (upper left), with normal collagen levels. The mice also regained normal body weight and survived (untreated controls did not).

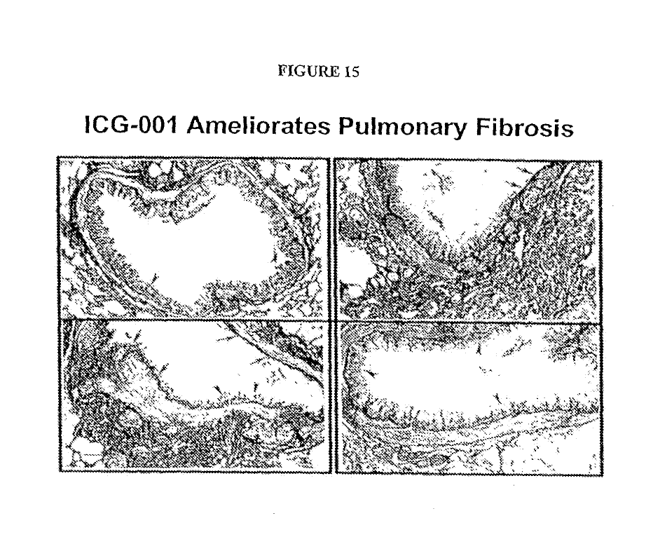

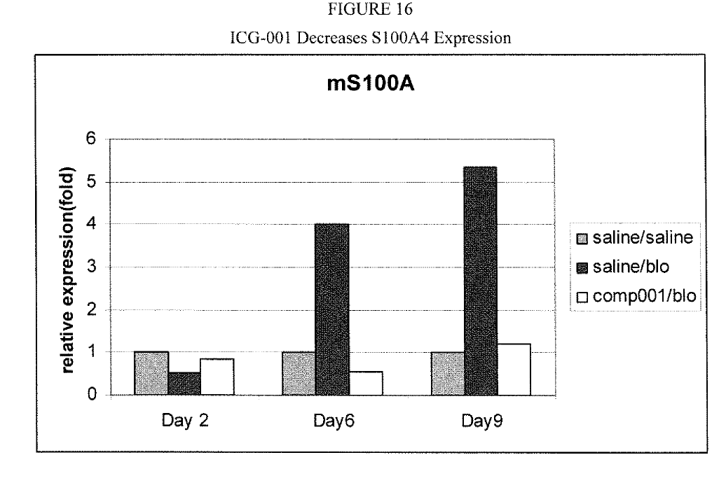

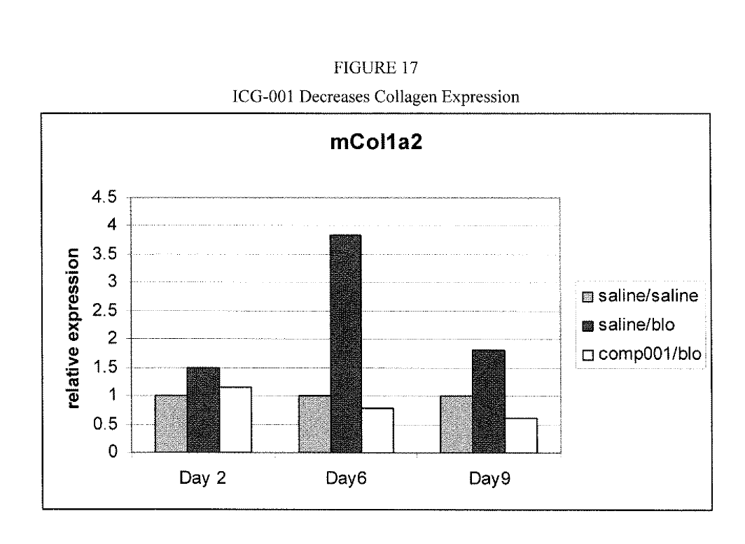

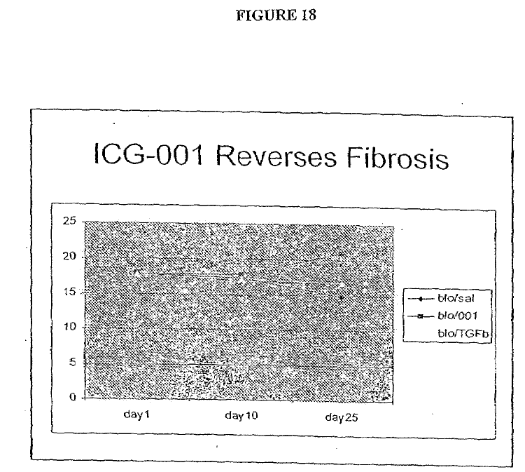

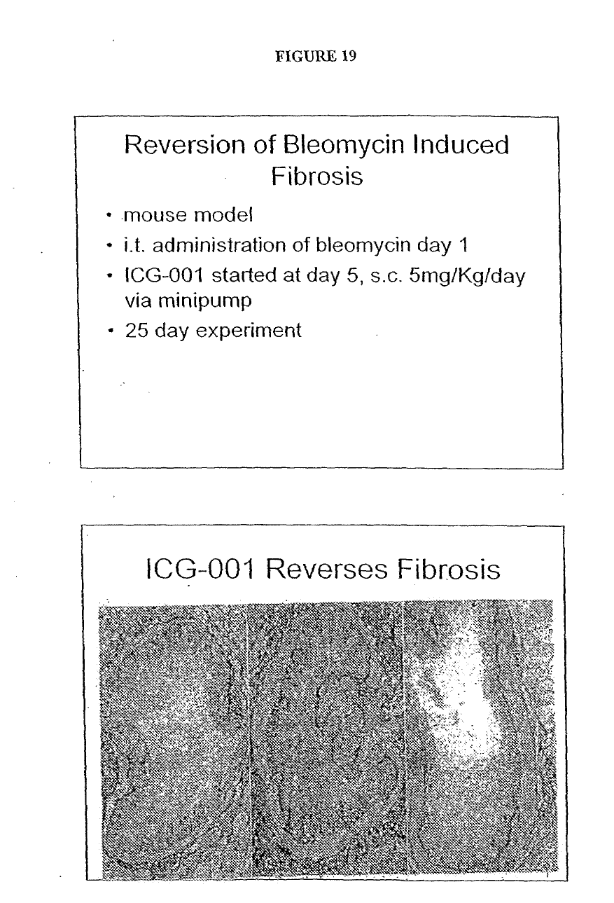

FIGS. 16 and 17 show RT-PCR data for S100A4 (FIG. 16) and collagen1A2 (FIG. 17), which are increased in the bleomycin treated mice (treated with saline control). Message is reduced essentially to negative control (i.e. saline/saline mice) levels by ICG-001 treatment (5 mgs/Kg/day s.c.). FIGS. 18 and 19 indicate that over the 25 days of treatment, ICG-001 reversed fibrosis.

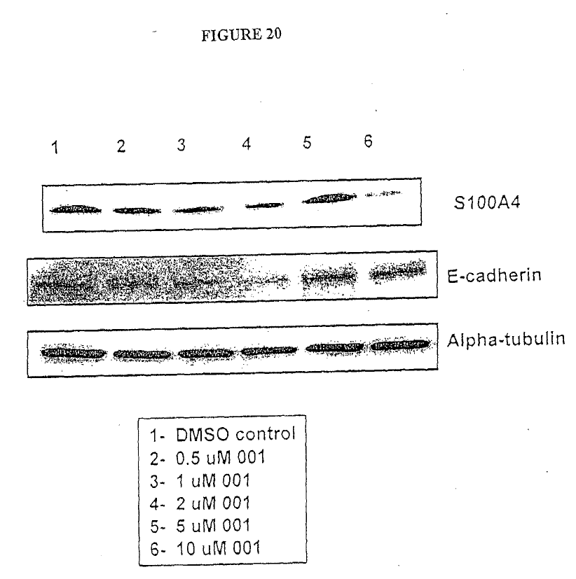

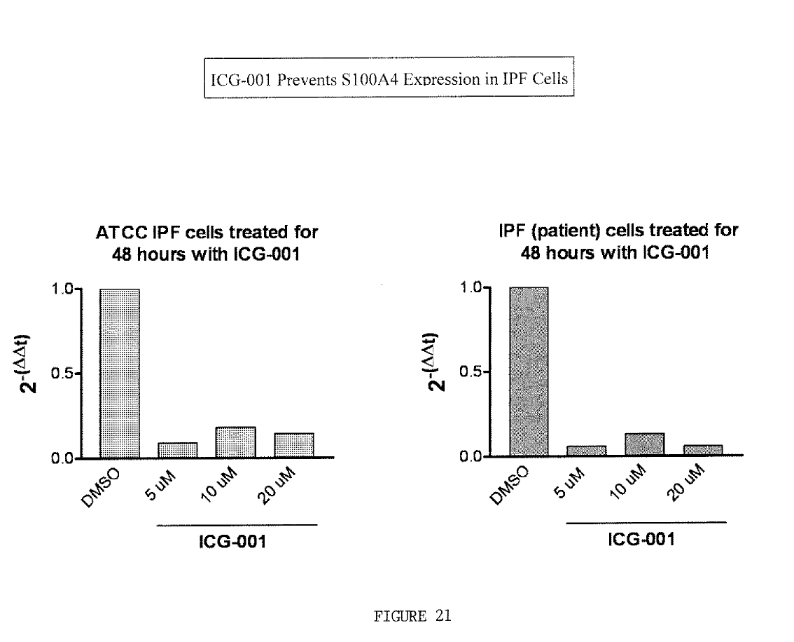

As shown in FIGS. 20 and 21, IPF patient fibroblasts were cultured in RPMI1640+10% PBS for 2 days and treated with ICG-001. Western blots for S100A4 (also know as FSP-1 or fibroblast specific protein-1) and E-Cadherin were performed on whole cell lysates (FIG. 20). ICG-001 decreased S100A4 expression (FIG. 21) and increased E-cadherin expression (this was also true at the mRNA level). These data demonstrate that ICG-001 mediates a mesenchymal to epithelial transition that is essential for normal healing, re-epithelialization and ameliorization of fibrosis.

### Example 3

### ICG-001 Increased Aquaporin Expression in Lung Epithelium

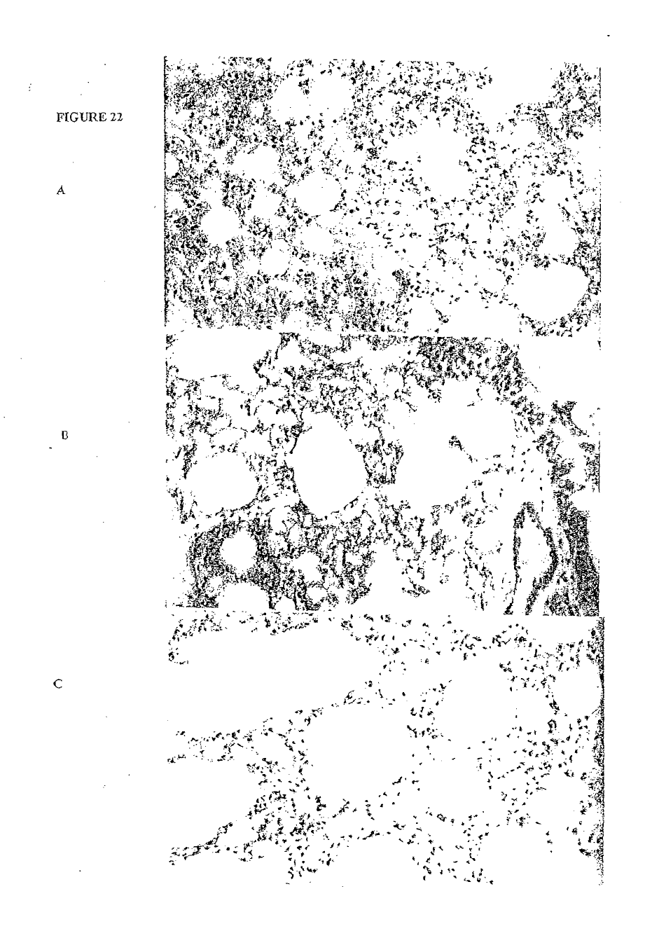

The aquaporins are water channels expressed in a variety of cell types. Aquaporin 5 is involved in the transportation of water across the apical surface of the alveolar epithelium and the epithelia of the submucosal glands in the upper airway and nasopharynx. (Krane, C. M., P.N.A.S. 98:14192-4, 2001; Yang, F. J. Biol. Chem. 278:32173-80, 2003). Because aquaporin 5 is a marker of Type 1 (differentiated) lung epithelium, its expression was assayed in lung tissue created with bleomycin (FIG. 22A), bleomycin and ICG-001 (FIG. 22B), and saline (FIG. 22C), using the animal model as described in Example 2 (FIG. 15), and immunostaining with an antibody specific for Aquaporin 5. Thus, aquaporin 5 expression was greatly increased by ICG-001.

### Example 4

### ICG-001 Prevented Interstitial Fibrosis and Alveolar Fibrosis

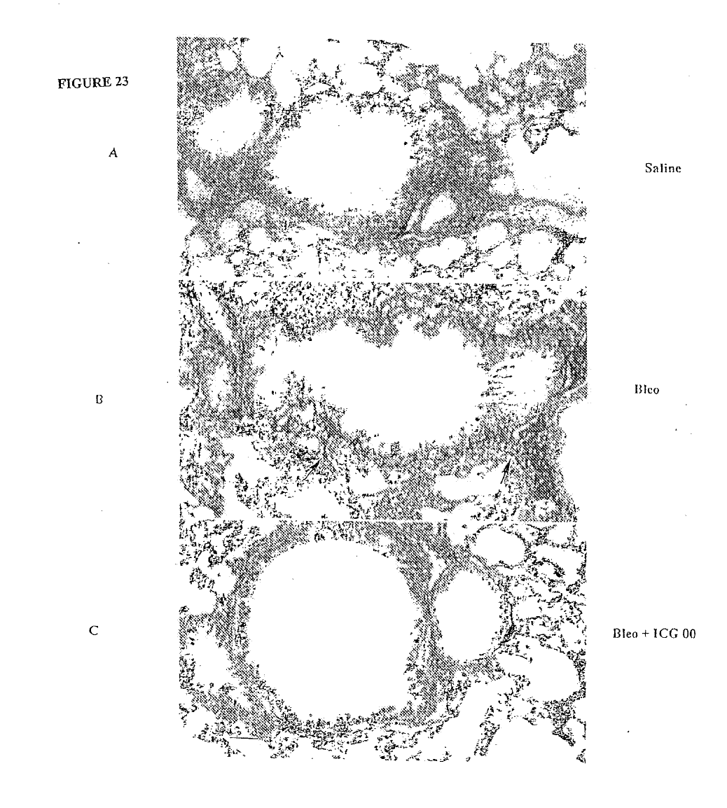

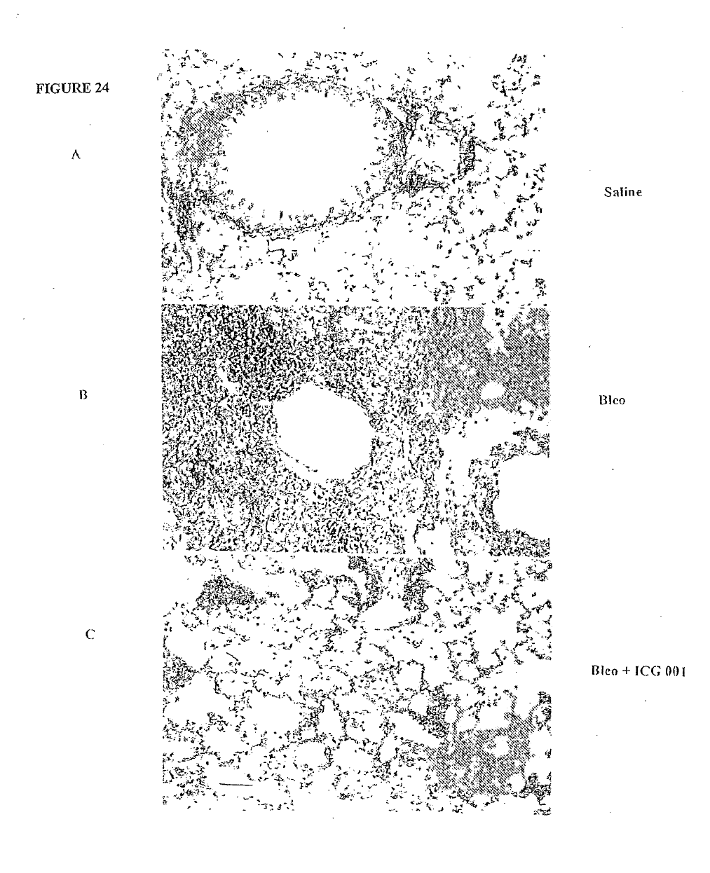

As indicated in FIG. 23, ICG-001 prevented interstitial fibrosis. FIG. 23A shows saline treatment; FIG. 23B shows bleomycin treatment; and FIG. 23C shows bleomycin and ICG-001 treatment. As indicated in FIG. 24, ICG-001 prevented alveolar fibrosis. FIG. 24A shows saline treatment; FIG. 24B shows bleomycin treatment; and FIG. 24C shows bleomycin and ICG-001 treatment. The procedures were performed using the animal model as described in Example 2 (FIG. 15), and the sectioned lungs were stained for collagen.

All patents, patent applications, provisional applications, and publications referred to or cited herein are incorporated by reference in their entirety, including all figures and tables, to the extent they are not inconsistent with the explicit teachings of this specification.

What is claimed is:

1. A method of treating a disease or pathological condition in a subject, said method comprising:
administering a compound to the subject under conditions effective to treat the disease or pathological condition in the subject, wherein the disease or pathological condition is selected from the group consisting of renal fibrosis, abdominal adhesions, radiation induced fibrosis, chemotherapy induced fibrosis, obliterative bronchiolitis, silicosis lesions, and Tenon's capsule fibroproliferation, interstitial lung disease, human fibrotic lung disease, human kidney disease, polycystic kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Alzheimer's disease, tuberous sclerosis complex, Lymphangioleiomyomatosis, pulmonary hypertension, atherosclerosis, restenosis, ulcerative colitis, rheumatoid arthritis, modulation of hair growth, and graft remodeling,
wherein said compound is selected from the group consisting of:
Compounds 1-2217,
a compound of formula (IA), or a stereoisomer, salt, or prodrug thereof: 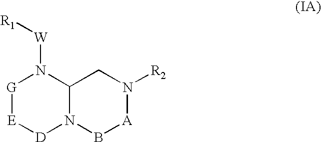
wherein
A is —(C═O)—(CHR₃)— or —(C═O)—;
B is —(R₅)— or —(CHR₆)—;
D is —(C═O)—(CHR₇)— —(C═O)—;
E is -(ZR₈)— or —(C═O)—, where Z is nitrogen or CH;
G is —(XR₉)ₙ—, —(CHR₁₀)—NR₆, —(C═O)—(XR₁₂)—, —(C═N—W—R₁)—, —(C═O)—, —(X—(C═O)—R₁₃)—, —(X—(C═O)—NR₁₃R₁₄)—, —(X—(SO₂)—R₁₃)—, or —(X—(C═O)—OR₁₃)—, where X is nitrogen or CH, and n=0 or 1;
W is —Y(C═O)—, —(C═O)—(NH)—, —(SO₂)—, —(CHR₁₄)—, —(C═O)—(NR₁₅)—, substituted or unsubstituted oxadiazole, substituted or unsubstituted triazole, substituted or unsubstituted thiadiazole, substituted or unsubstituted 4,5-dihydrooxazole, substituted or unsubstituted 4,5-dihydrothiazole, substituted or unsubstituted 4,5-dihydroimidizole, or nothing, where Y is oxygen or sulfur; and
R₁, R₂, R₃, R₄, R₅, R₆, R₇, R₈, R₉ R₁₀, R₁₁, R₁₂, R₁₃, R₁₄, and R₁₅ are the same or different and independently selected from the group consisting of an amino acid side chain moiety or derivative thereof, the remainder of the molecule, a linker, and a solid support;
provided that where B is —(CHR₆)— and W is —Y(C═O)—, —(C═O)—(NH)—, —(SO₂)—, —(CHR₁₄)—, or —(C═O)—(NR₁₅)—, G cannot be —(CHR₉)—, —(NR₉)—, —(C═O)—(CHR₁₂)—, —(C═O)—(NR₁₂)—, or no atom at all; and
a compound of formula (IB), or a stereoisomer, salt, or prodrug thereof: 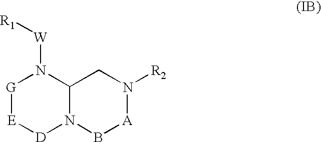
wherein
A is —(C═O)—(CHR₃)— or —(C═O)—;
B is —(NR₅)— or —(CHR₆)—;
D is —(C═O)—(CHR₇)— or —(C═O)—;
E is -(ZR₈)— or —(C═O)—, where Z is nitrogen or CH;
G is —(XR₉)ₙ—, —(CHR₁₀)—(NR₆)—, —(C═O)—(XR₁₂)—, —(C═O)—, —(X—(C═O)—R₁₃)—, —(X—(C═O)—NR₁₃R₁₄)—, —(X—(SO₂)—R₁₃)—, or —(X—(C═O)—OR₁₃)—, where X is nitrogen or CH, and n=0 or 1;
W is —Y(C═O)—, —(C═O)—(NH)—, —(SO₂)—, —(CHR₁₄)—, —(C═O)—(NR₁₅)—, substituted or unsubstituted oxadiazole, substituted or unsubstituted triazole, substituted or unsubstituted thiadiazole, substituted or unsubstituted 4,5-dihydrooxazole, substituted or unsubstituted 4,5-dihydrothiazole, substituted or unsubstituted 4,5-dihydroimidizole, or nothing, where Y is oxygen or sulfur; and
R₁, R₂, R₃, R₄, R₅, R₆, R₇, R₈, R₉ R₁₀, R₁₁, R₁₂, R₁₃, R₁₄, and R₁₅ are the same or different and independently selected from the group consisting of an amino acid side chain moiety or derivative thereof, the remainder of the molecule, a linker, and a solid support.

2. The method according to claim 1, wherein the compound is selected from Compounds 1-2217 and the disease or pathological condition is selected from the group consisting of renal fibrosis, abdominal adhesions, radiation induced fibrosis, chemotherapy induced fibrosis, obliterative bronchiolitis, silicosis lesions, and Tenon's capsule fibroproliferation.

3. The method according to claim 1, wherein the compound is a compound of formula (IA) or a stereoisomer, salt, or prodrug thereof, and the disease or pathological condition is selected from the group consisting of interstitial lung disease, human fibrotic lung disease, human kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Lymphangioleiomyomatosis, pulmonary hypertension, atherosclerosis, and graft remodeling.

4. The method according to claim 1, wherein the disease or pathological condition is selected from the group consisting of interstitial lung disease, human fibrotic lung disease, human kidney disease, polycystic kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Alzheimer's disease, tuberous sclerosis complex, Lymphangioleiomyomatosis, pulmonary hypertension, atherosclerosis, restenosis, ulcerative colitis, rheumatoid arthritis, modulation of hair growth, and graft remodeling; and the compound is selected from the group consisting of Compounds 1-2217, and a compound of formula (IB) wherein R₁, R₂, R₃, R₄, R₅, R₆, R₇, R₈, R₉, R₁₀, R₁₁, R₁₂, R₁₃, R₁₄, and R₁₅ are independently selected from the group consisting of aminoC₂₋₅alkyl, guanidinoC₂₋₅alkyl, C₁₋₄alkylguanidinoC₂₋₅alkyl, diC₁₋₄alkylguanidino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), pyridyl, substituted pyridyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoroC₁₋₄alkyl, C₁₋₄alkyl, C₁₋₃alkoxy, nitro, carboxy, cyano, sulfuryl, and hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, and methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl, and 4-aminocyclohexylC₀₋₂alkyl.

5. The method according to claim 1, wherein the compound is administered to the subject orally, transdermally, intravenously, topically, by inhalation, rectally, or by treated stent, wherein the stent comprises or is coated with the compound.

6. The method according to claim 5, wherein the compound is administered to the subject orally.

7. The method according to claim 1, wherein the compound is administered to the subject by sustained release.

8. The method according to claim 1, wherein the compound is contained in a pharmaceutical composition in a form selected from the group consisting of capsules, tablets, powders, granules, syrups, injectable fluids, creams, ointments, hydrophilic ointments, inhalable fluids, eye drops, suppositories, and treated stent, wherein the stent comprises or is coated with the pharmaceutical composition.

9. The method according to claim 1, wherein the compound is a compound of formula (IA) or a stereoisomer, salt, or prodrug thereof, wherein R₁, R₂, R₃, R₄, R₅, R₆, R₇, R₈, R₉, R₁₀, R₁₁, R₁₂, R₁₃, R₁₄, and R₁₅ are independently selected from the group consisting of aminoC₂₋₅alkyl, guanidinoC₂₋₅alkyl, C₁₋₄alkylguanidinoC₂₋₅alkyl, diC₁₋₄alkylguaindino-C₂₋₅alkyl, amidinoC₂₋₅alkyl, C₁₋₄alkylamidinoC₂₋₅alkyl, diC₁₋₄alkylamidinoC₂₋₅alkyl, C₁₋₃alkoxy, phenyl, substituted phenyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), benzyl, substituted benzyl (where the substituents on the benzyl are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), naphthyl, substituted naphthyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), bis-phenyl methyl, substituted bis-phenyl methyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), pyridyl, substituted pyridyl (where the substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), pyridylC₁₋₄alkyl, substituted pyridylC₁₋₄alkyl (where the pyridine substituents are independently selected from one or more of amino, amidino, guanidino, hydrazino, amidrazonyl, C₁₋₄alkylamino, C₁₋₄dialkylamino, halogen, perfluoroC₁₋₄alkyl, C₁₋₄alkyl, C₁₋₃alkoxy, nitro, carboxy, cyano, sulfuryl, and hydroxyl), pyrimidylC₁₋₄alkyl, substituted pyrimidylC₁₋₄alkyl (where the pyrimidine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), triazin-2-yl-C₁₋₄alkyl, substituted triazin-2-yl-C₁₋₄alkyl (where the triazine substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, and hydroxyl), imidazoC₁₋₄alkyl, substituted imidazol C₁₋₄alkyl (where the imidazole substituents are independently selected from one or more of amino, amidino, guanidino, hydrazino, amidrazonyl, C₁₋₄alkylamino, C₁₋₄dialkylamino, halogen, perfluoro C₁₋₄alkyl, C₁₋₄alkyl, C₁₋₃alkoxy, nitro, carboxy, cyano, sulfuryl, hydroxyl, and methyl), imidazolinylC₁₋₄alkyl, N-amidinopiperazinyl-N—C₀₋₄alkyl, hydroxyC₂₋₅alkyl, C₁₋₅alkylaminoC₂₋₅alkyl, hydroxyC₂₋₅alkyl, C₁₋₅alkylaminoC₂₋₅alkyl, C₁₋₅dialkylaminoC₂₋₅alkyl, N-amidinopiperidinylC₁₋₄alkyl, and 4-aminocyclohexylC₀₋₂alkyl.

10. The method according to claim 1, wherein the compound is a compound of formula (IA) having the following general formula (III), or a stereoisomer, salt, or prodrug thereof: 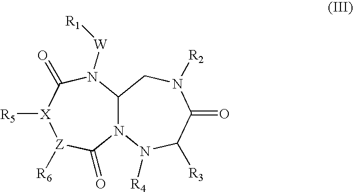
wherein when Z is CH, X is nitrogen.

11. A method of treating a disease or pathological condition in a subject, said method comprising:
administering a compound to the subject under conditions effective to treat the disease or pathological condition in the subject, wherein the disease or pathological condition is selected from the group consisting of renal fibrosis, abdominal adhesions, radiation induced fibrosis, chemotherapy induced fibrosis, obliterative bronchiolitis, silicosis lesions, and Tenon's capsule fibroproliferation, interstitial lung disease, human fibrotic lung disease, human kidney disease, polycystic kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Alzheimer's disease, tuberous sclerosis complex, Lymphangioleiomyomatosis, pulmonary hypertension, atherosclerosis, restenosis, ulcerative colitis, rheumatoid arthritis, modulation of hair growth, and graft remodeling,
wherein said compound is a compound of formula (IV), or a stereoisomer, salt, or prodrug thereof: 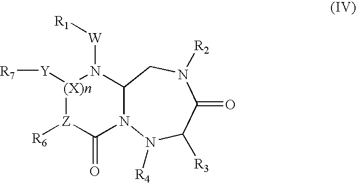
wherein
W is —V(C═O)—, —(C═O)—(NH)—, —(SO₂)—, —(CHR₁₄)—, —(C═O)—(NR₁₅)—, substituted or unsubstituted oxadiazole, substituted or unsubstituted triazole, substituted or unsubstituted thiadiazole, substituted or unsubstituted 4,5-dihydrooxazole, substituted or unsubstituted 4,5-dihydrothiazole, substituted or unsubstituted 4,5-dihydroimidizole, or nothing, where V is oxygen or sulfur; and
X is nitrogen;
Y is —C═O—, —(C═O)—O—, —(C═O)—(NR₈)—, —SO₂—, or nothing;
Z is nitrogen or CH;
n=0 or 1; and
R₁, R₂, R₃, R₄, R₆, R₇, R₈, R₁₄, and R₁₅ are the same or different and independently selected from the group consisting of an amino acid side chain moiety or derivative thereof, the remainder of the molecule, a linker, and a solid support;
with the proviso that when Z is nitrogen, n is zero, and when Z is CH, n is 1.

12. A method of treating a disease or pathological condition in a subject, said method comprising:
administering a compound to the subject under conditions effective to treat the disease or pathological condition in the subject, wherein the disease or pathological condition is selected from the group consisting of renal fibrosis, abdominal adhesions, radiation induced fibrosis, chemotherapy induced fibrosis, obliterative bronchiolitis, silicosis lesions, and Tenon's capsule fibroproliferation, interstitial lung disease, human fibrotic lung disease, human kidney disease, polycystic kidney disease, renal fibrotic disease, glomerular nephritis, liver cirrhosis, nephritis associated with systemic lupus, peritoneal fibrosis, liver fibrosis, polycystic ovarian syndrome, myocardial fibrosis, pulmonary fibrosis, Grave's ophthalmopathy, glaucoma, scarring, skin lesions, diabetic retinopathy, scleroderma, Alzheimer's disease, tuberous sclerosis complex, Lymphangioleiomyomatosis, pulmonary hypertension, atherosclerosis, restenosis, ulcerative colitis, rheumatoid arthritis, modulation of hair growth, and graft remodeling,
wherein said compound is a compound of formula (V), or a stereoisomer, salt, or prodrug thereof: 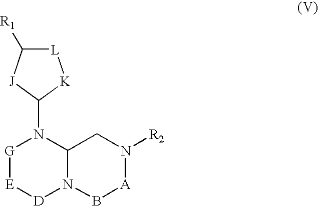
wherein
A is —(C═O)—(CHR₃)— or —(C═O)—;
B is —(NR₅)— or —(CHR₆)—;
D is —(C═O)—(CHR₇)— or —(C═O)—;
E is -(ZR₈)— or —(C═O)—, where Z is nitrogen or CH;
G is —(XR₉)ₙ—, —(CHR₁₀)—(NR₆)—, —(C═O)—(XR₁₂)—, —(C═N—W—R₁)—, —(C═O)—, —(X—(C═O)—R₁₃—, —(X—(C═O)—NR₁₃R₁₄)—, —(X—(SO₂)—R₁₃)—, or —(X—(C═O)—OR₁₃)—, X is nitrogen or CH, and n=0 or 1;
K is nitrogen, oxygen, or sulfur;
L is nitrogen, oxygen, —(CH)═, or —(CH₂)—;
J is nitrogen, oxygen, or sulfur; and
R₁, R₂, R₅, R₆, R₇, R₈, R₉, R₁₀, R₁₂, R₁₃, and R₁₄ are independently selected from an amino acid side chain moiety.
